(19) United States
(12) Patent Application Publication
(10) Pub. No.: US 2025/0114305 A1
(43) Pub. Date: Apr. 10, 2025

(54) LIPIDIC COMPOUNDS, AND USES THEREOF

(71) Applicant: SANOFI, Paris (FR)

(72) Inventors: Pascal DESMAZEAU, Paris (FR); Luc EVEN, Paris (FR); Nathalie RAMEIX, Paris (FR)

(21) Appl. No.: 18/729,020

(22) PCT Filed: Jan. 16, 2023
(86) PCT No.: PCT/EP2023/050891

§ 371 Date: Jul. 15, 2024

(30) Foreign Application Priority Data
Jan. 17, 2022 (EP) 22305043.6

(51) Int. Cl. A61K 9/1272; A61K 9/51; A61K 39/00; A61K 39/145; A61P 31/16; C07C 229/12; C07C 237/08; C07D 207/16; C07D 211/34; C07D 211/62; C07D 295/15

(52) U.S. Cl. CPC A61K 9/1272; A61K 9/5123; A61K 39/145; A61P 31/16; C07C 229/12; C07C 237/08; C07D 207/16; C07D 211/34; C07D 211/62; C07D 295/15; A61K 2039/53; A61K 2039/55555; A61K 2039/575

(57) ABSTRACT

The disclosure relates to lipidic compounds of formula (I): A—(CH₂)ₙ—CX—B—Z—R1 (I) wherein R1 represents a lipophilic or hydrophobic tail-group, wherein RI is an optionally substituted, branched, saturated or unsaturated, C₁₀ to C₅₅ hydrocarbon radical, and which hydrocarbon skeleton that is optionally interrupted by one or several atoms of oxygen or nitrogen and/or one or several moiety —(C═O)—, —O—(C═O)— or —(C═O)—O— and which one nitrogen atom, if present in the skeleton, can be linked, directly or not, to said Z radical; Z is a spacer arm; B represents O or NH; X is O or S; n is 0, 1, 2, 3, 4, 5 or 6; and A represents (i) R2R3N-, (ii) NR2R3-Alk-Y-in which Y is O or N, Alk is an alkylene moiety in C₂ to C₆ and R2 and R3 represent independently of each other a linear or branched (C₁-C₆) alkyl group, or (iii) a 4- to 8-membered saturated heterocyclic radical comprising 3 to 7 carbon atoms and 1 or 2 nitrogen atoms, said 4- to 8-membered saturated heterocyclic radical being linked to the rest of the molecule by a carbon atom or a nitrogen atom and being optionally substituted by 1 to 4 substituents, independently of each other, selected from a linear or branched (C₁-C₆) alkyl group; or one of its pharmaceutically acceptable salts thereof; and with said compound that is in all the possible racemic, enantiomeric and diastereoisomeric isomer forms.

### TECHNICAL FIELD

The present disclosure is in the field of novel lipid compounds. The novel lipidic compounds are ionizable cationic lipids. The lipidic compounds can be used, for example in combination with other lipid components, such as neutral lipids, structural lipids and polymer conjugated lipids, to form lipid nanoparticles. The lipid nanoparticles can be used for the delivery of therapeutic agents, such as nucleic acid.

### TECHNICAL BACKGROUND

The polynucleotide therapeutics field has seen remarkable progress over the recent years. Polynucleotides include various nucleic acids-based compounds such as messenger RNA (mRNA), antisense oligonucleotides, ribozymes, DNAzymes, plasmids, or immune stimulating nucleic acids. Some nucleic acids, such as mRNA, plasmids and ssDNA can be used to induce the expression of specific cellular products useful in the treatment of, for example, diseases related to a deficiency of a protein or enzyme, or for the expression of a vaccine antigen to induce specific immune responses. The therapeutic applications of translatable nucleotide delivery are extremely broad as constructs can be synthesized to produce any chosen protein sequence, whether or not indigenous to the system. The expression products of the nucleic acid can augment existing levels of protein, replace missing or non-functional versions of a protein, or introduce new protein and associated functionality in a cell or organism or expose to a foreign protein in order to induce a specific immune response.

However, there are many challenges associated with the delivery of polynucleotides to affect a desired response in a biological system and the effective delivery of polynucleotides to their intracellular sites of action remains a major issue. To be efficiently delivered to their site of action, the polynucleotides must be (i) protected from enzymatic and non-enzymatic degradation, (ii) appropriately distributed in the biologic compartment of interest, (iii) effectively and efficiently internalized by the targeted cells, and then (iv) delivered to the intracellular compartment where the relevant translation machinery resides.

Lipid nanoparticles (LNPs) have proven efficient to deliver various types of therapeutic active agents into cells (Thi et al., Vaccines, 2021, 9 (4), 359). For example, LNPs containing nucleic acids, such as LNP-mRNA, have attracted a great interest and have recently proven their efficacy and safety in vaccine fields and have proven dramatically important in the management of the Covid-19 pandemic (Reichmuth et al., Therapeutic delivery, 2016, 7 (5), 319-334; Khurana et al., Nano today, 2021, 38, 101142).

For example, lipid nanoparticles formed from cationic lipids formulated with other lipid components, such as neutral lipids, cholesterol, and PEGylated lipids have been used to protect the polynucleotide from degradation and facilitate its cellular uptake.

While lipid nanoparticle-based vehicles that comprise a cationic lipid component have shown promising results with regard to encapsulation, stability and site localization, there remains a great need for improvement of lipid nanoparticle-based delivery systems.

There remains a need for improved cationic and ionizable lipids that demonstrate improved pharmacokinetic properties, and which are capable of delivering various types of polynucleotides to a wide variety cell types and tissues with enhanced efficiency.

There also remains a need for novel cationic ionizable lipids having reduced toxicity and are capable of efficiently delivering encapsulated polynucleotides to targeted cells, tissues and organs.

There is a need for novel cationic ionizable lipids being able to be easily eliminated in vivo, after administration and having reduced toxicity.

There is a need for biodegradable cationic ionizable lipids.

Improved cationic lipids and lipid nanoparticles for the delivery of polynucleotides would also provide optimal polynucleotide/lipid ratios, protect the polynucleotides from degradation and clearance in serum, be suitable for systemic or local delivery, and provide intracellular delivery of the polynucleotide.

In addition, the lipid-polynucleotide particles should be well-tolerated and provide an adequate therapeutic index, such that patient treatment at an effective dose of the polynucleotide is not associated with unacceptable toxicity and/or risk to the patient.

In addition, the lipid-nucleic acid particles should be stable as liquid formulations when stored at 4-8° C. for long periods of time in a pharmaceutically acceptable buffer.

The present disclosure aims at satisfying all or part of those needs.

### SUMMARY

According to one of its objects, the present disclosure relates to a lipidic compound of formula (I):

A—(CH₂)ₙ—CX—B—Z—R1 (I)
wherein:
   R1 is a C₁₀ to C₅₇, or C₁₀ to C₅₅, lipophilic or hydrophobic tail-group;
   Z may be a spacer arm having from 2 to 24, for instance from 2 to 18, for example from 4 to 12 carbon atoms in a branched or unbranched linear saturated or unsaturated hydrocarbon chain, said chain that is interrupted by one or several atoms of oxygen and/or moieties selected among —S—S—; —(O═C)—; —(C═O)—O—; —O—(O═C)—; —S—; —NH—, —NH—(O═C)—; —(O═C)—NH— and —NH—(C═O)—O— and for instance by —(C═O)—O—; —O—(O═C)— and —NH—(C═O)—O— and optionally having an oxygen atom or a moiety selected among —NH—(O═C)—*—O—(O═C)—*; —(C═O)—O—*; and —(O═C)— to its end linked to the hydrophobic tail-group, with * indicating the single bond linking said moiety to the hydrophobic tail-group;
   B is an oxygen atom or a —NH— group;
   X may be an oxygen atom or a sulfur atom;
   n may be 0, 1, 2, 3, 4, 5 or 6; and
   A is a group selected in the group consisting of:
   a R2R3N-group in which R2 and R3 is independently of each other a linear or branched (C₁-C₆) alkyl group;
   a NR2R3-Alk-Y— group in which Y is an oxygen or a nitrogen atom, Alk may be an alkylene moiety in C₂ to C₆ and R2 and R3 is independently of each other a linear or branched (C₁-C₆) alkyl group;
   a 4- to 8-membered saturated heterocyclic radical comprising 3 to 7 carbon atoms and 1 or 2 nitrogen atoms, said 4- to 8-membered saturated heterocyclic radical being linked to the rest of the molecule by a carbon atom or a nitrogen atom and being optionally substituted by 1 to 4 substituents, independently of each other, selected from a linear or branched (C₁-C₆) alkyl group;
or one of its pharmaceutically acceptable salts thereof; and with said compound that is in all the possible racemic, enantiomeric and diastereoisomeric isomer forms.

According to another of its object, the disclosure relates to a lipidic compound of formula (I):

A—(CH₂)ₙ—CX—B—Z—R1 (I)
wherein:
   R1 is a C₁₀ to C₅₇, or C₁₀ to C₅₅ lipophilic or hydrophobic tail-group, wherein R1 is an optionally substituted, branched or unbranched linear, saturated or unsaturated, C₁₀ to C₅₇, or C₁₀ to C₅₅, hydrocarbon radical, and which hydrocarbon skeleton that is optionally interrupted by one or several atoms of oxygen or nitrogen and/or one or several moiety —(C═O)—, —O—(C═O)— or —(C═O)—O— and which one nitrogen atom, if present in the skeleton, can be linked, directly or not, to said Z radical;
   Z may be a spacer arm having from 2 to 24, for instance from 2 to 18, for example from 4 to 12 carbon atoms in an unbranched linear saturated or unsaturated hydrocarbon chain, said chain that is interrupted by one or several atoms of oxygen and/or moieties selected among —S—S—; —(O═C)—; —(C═O)—O—; —O—(O═C)—; —S—; —NH—, —NH—(O═C)—; —(O═C)—NH— and —NH—(C═O)—O— and for instance by —(C═O)—O—; —O—(O═C)— and —NH—(C═O)—O— and optionally having an oxygen atom or a moiety selected among —NH—(O═C)—*—O—(O═C)—*; —(C═O)—O—*; and —(O═C)— to its end linked to the hydrophobic tail-group, with * indicating the single bond linking said moiety to the hydrophobic tail-group;
   B is an oxygen atom or a —NH— group;
   X may be an oxygen atom or a sulfur atom;
   n may be 0, 1, 2, 3, 4, 5 or 6; and
   A is a group selected in the group consisting of:
   a R2R3N-group in which R2 and R3 is independently of each other a linear or branched (C₁-C₆) alkyl group;
   a NR2R3-Alk-Y-group in which Y is an oxygen or a nitrogen atom, Alk may be an alkylene moiety in C₂ to C₆ and R2 and R3 is independently of each other a linear or branched (C₁-C₆) alkyl group;
   a 4- to 8-membered saturated heterocyclic radical comprising 3 to 7 carbon atoms and 1 or 2 nitrogen atoms, said 4- to 8-membered saturated heterocyclic radical being linked to the rest of the molecule by a carbon atom or a nitrogen atom and being optionally substituted by 1 to 4 substituents, independently of each other, selected from a linear or branched (C₁-C₆) alkyl group;
or one of its pharmaceutically acceptable salts thereof; and with said compound that is in all the possible racemic, enantiomeric and diastereoisomeric isomer forms.

Surprisingly, and as detailed in the Examples section, the inventors have observed that the novel lipidic compounds as disclosed herein enable the formulation of improved compositions, such as lipid nanoparticles, for the in vitro and in vivo delivery of mRNA and/or other oligonucleotides or oligonucleotides.

The LNPs obtained with the lipidic compounds of the disclosure were shown to be of reduced and homogeneous size and were able to induce strong protein expression. 10

The improved lipid nanoparticles are useful for expression of protein encoded by mRNA. The lipid nanoparticles as disclosed herein may be used for regulation, up-regulation or down-regulation, of protein expression by delivering either miRNA or miRNA inhibitors for modulating expression of endogenous protein, or mRNA or plasmids for expression of transgenes. Also, the lipid nanoparticles as disclosed herein may be used for inducing a pharmacological effect resulting from expression of a protein or a protection against infection through delivery of mRNA encoding for a suitable antigen, such as an influenza antigen, or an antibody.

Also, the lipid nanoparticles as disclosed herein may be used for inducing a pharmacological effect resulting from expression of a protein, such as erythropoietin (EPO), useful for the treatment of metabolic diseases or diseases resulting from protein deficiency.

A compound disclosed herein may be under a cationic form.

In a compound disclosed herein, R1 is an optionally substituted, branched or unbranched linear, saturated or unsaturated, C₁₀ to C₅₅ hydrocarbon radical, and which hydrocarbon skeleton that is optionally interrupted by one or several atoms of oxygen or nitrogen and/or one or several moiety —CO—, —O—CO— or —CO—O— and which one nitrogen atom, if present in the skeleton, can be linked, directly or not, to said Z radical as defined herein.

In a compound disclosed herein, R1 may be a group selected in the group consisting of:

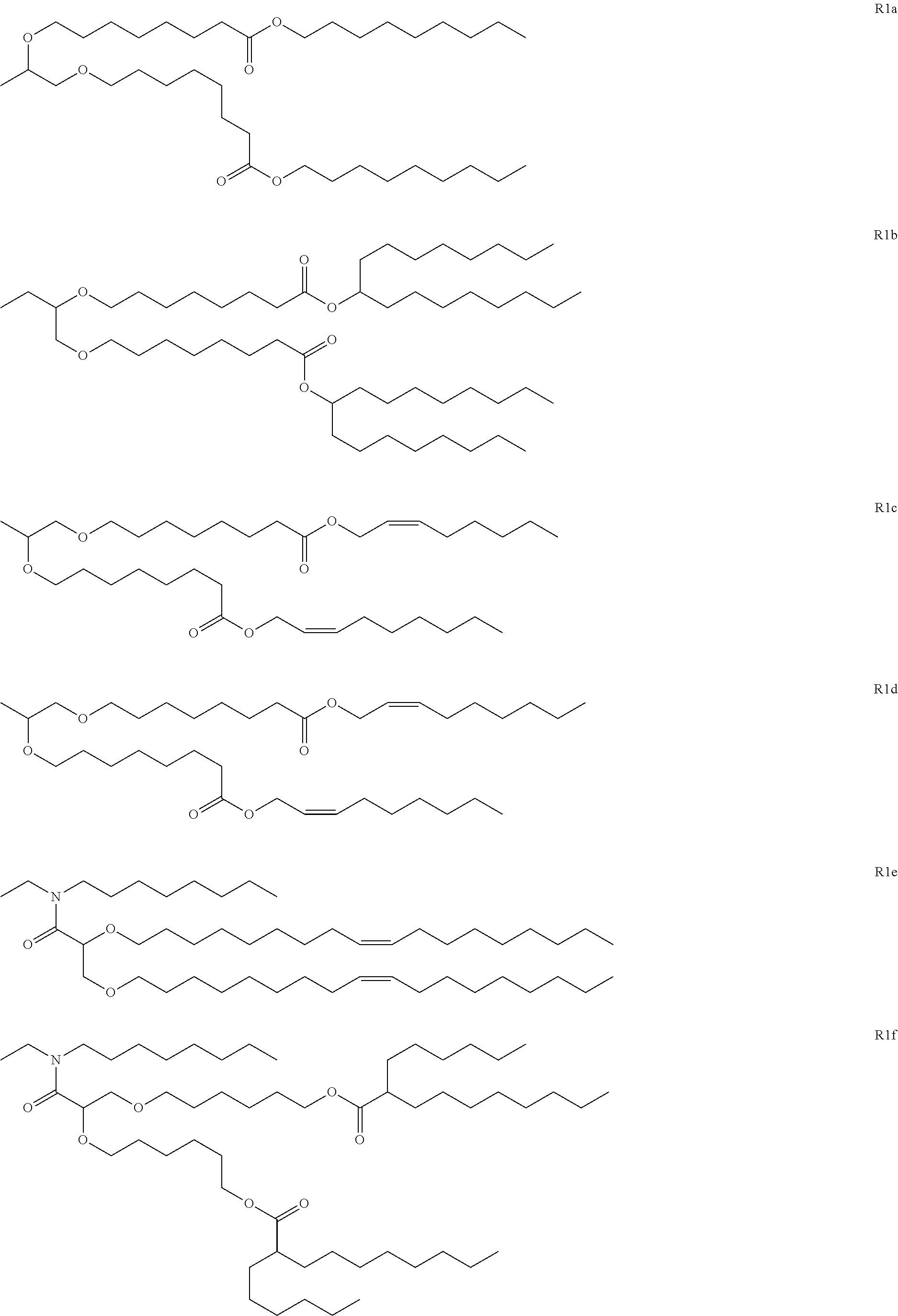

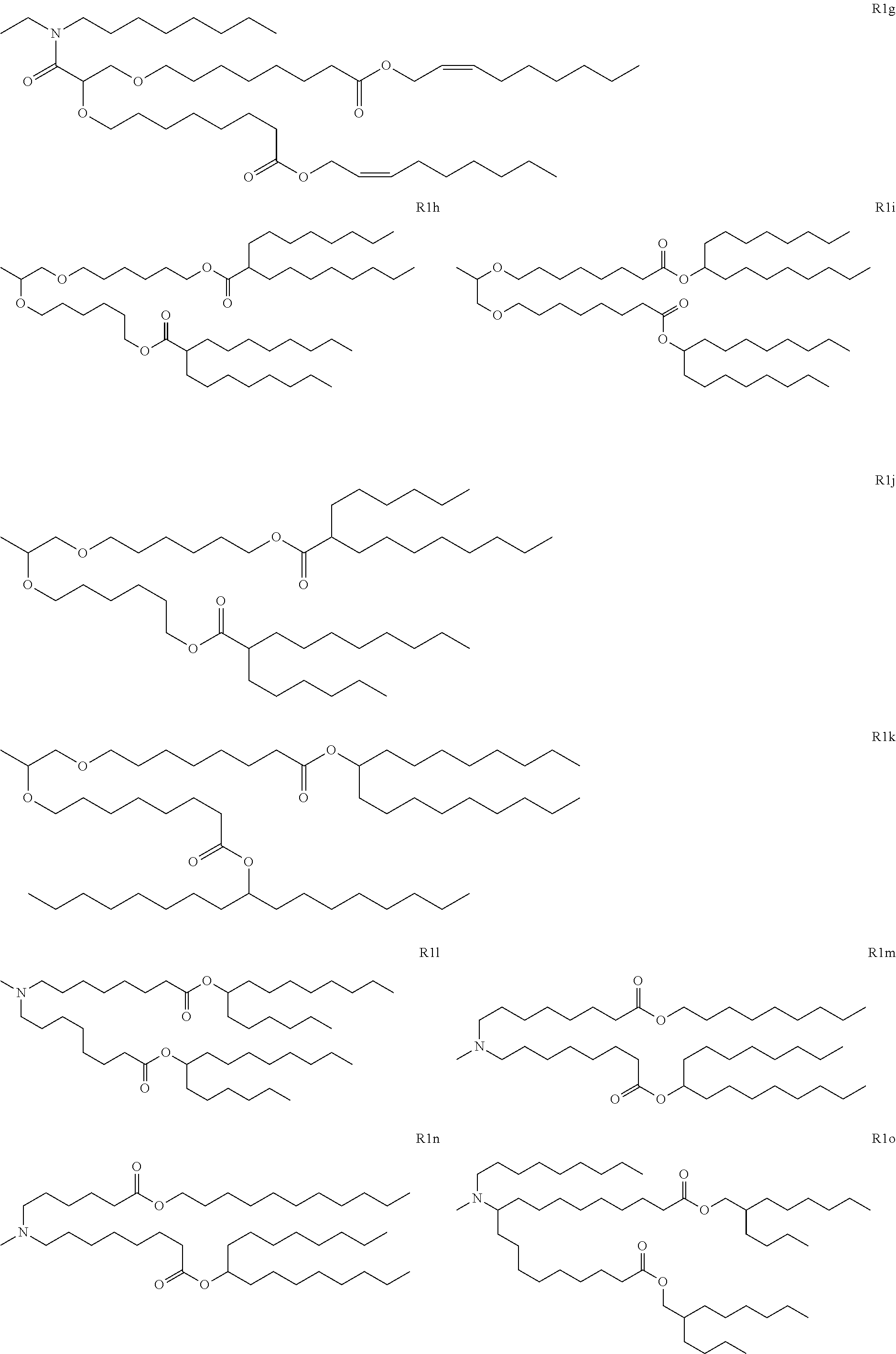

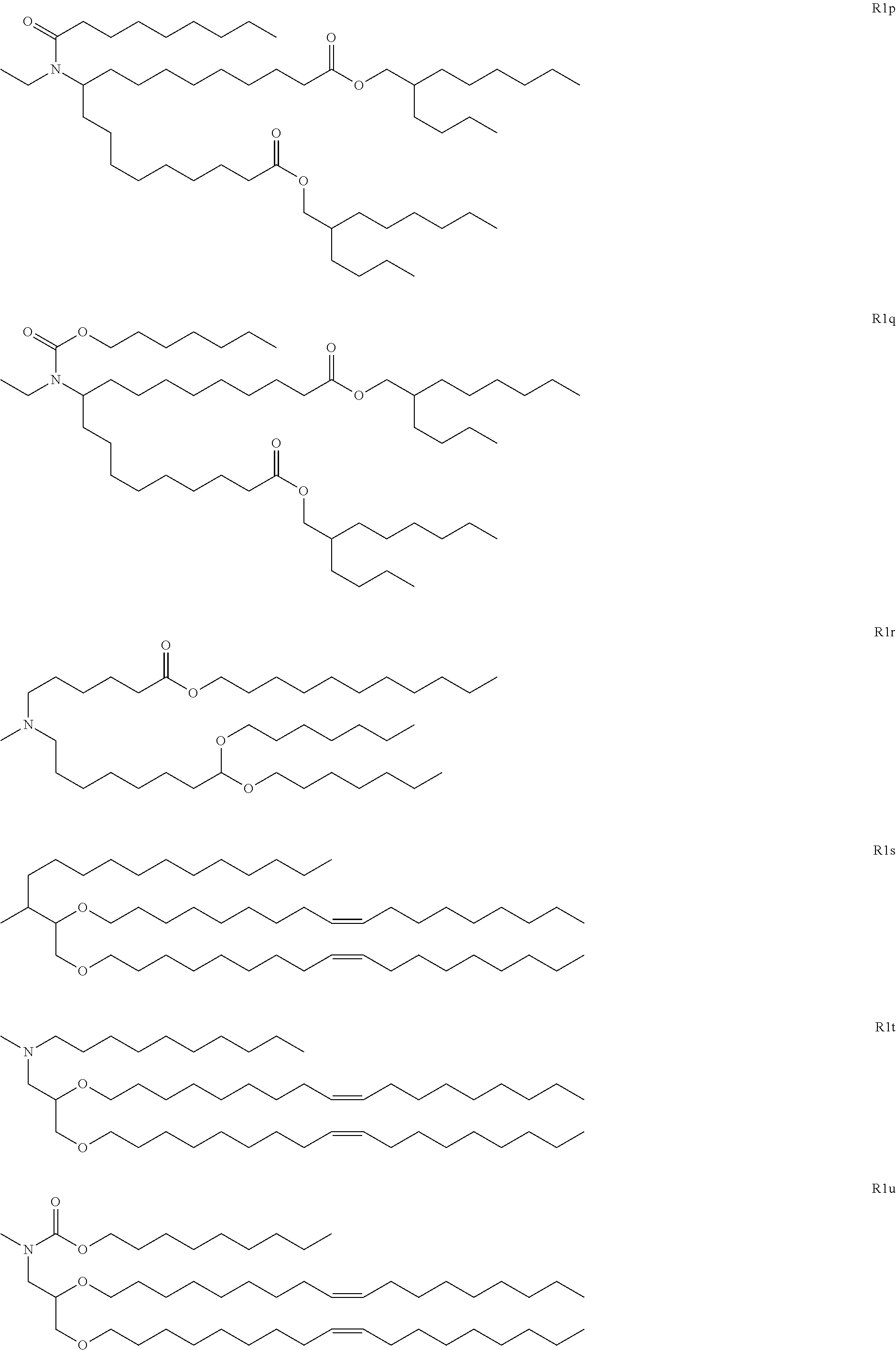

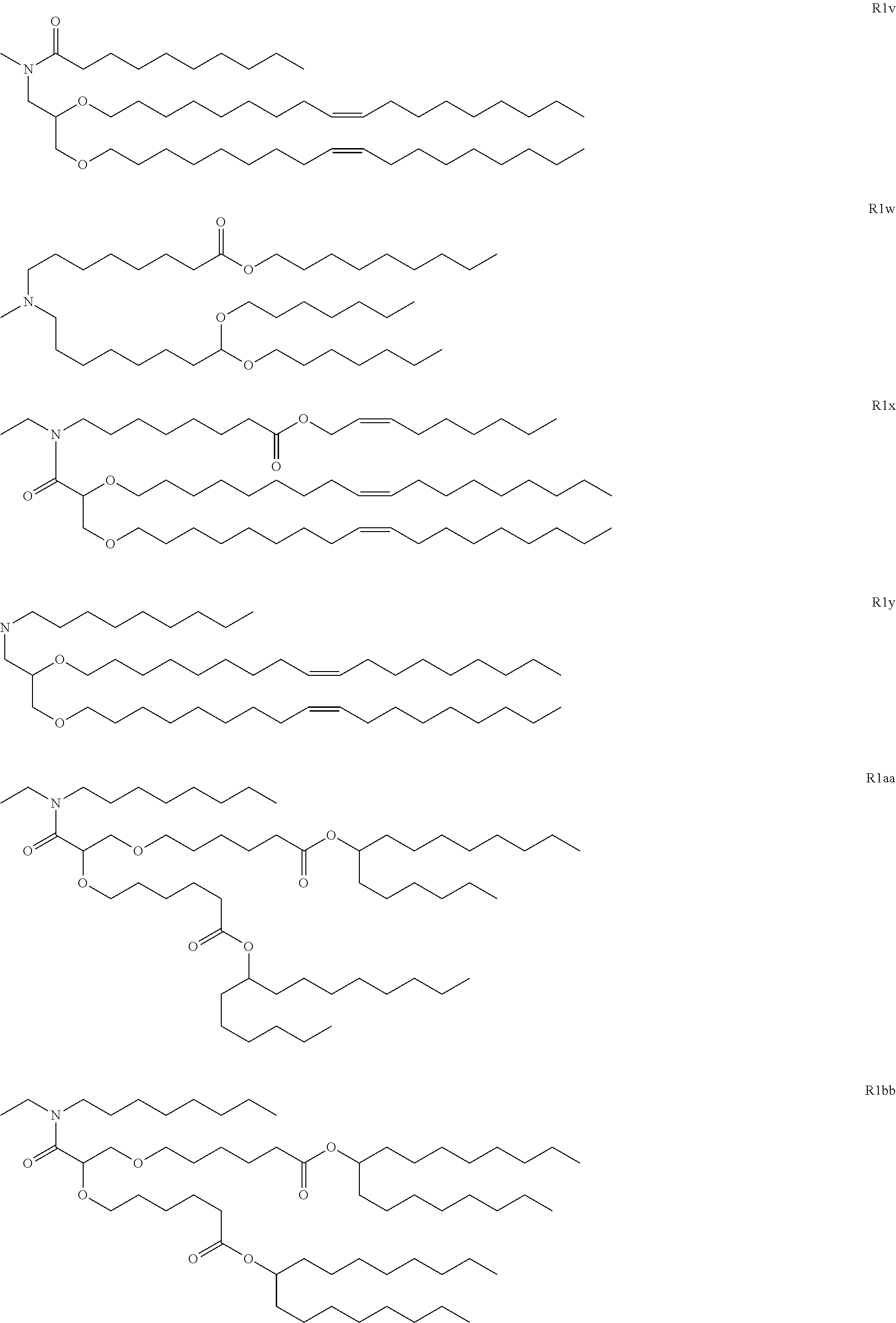

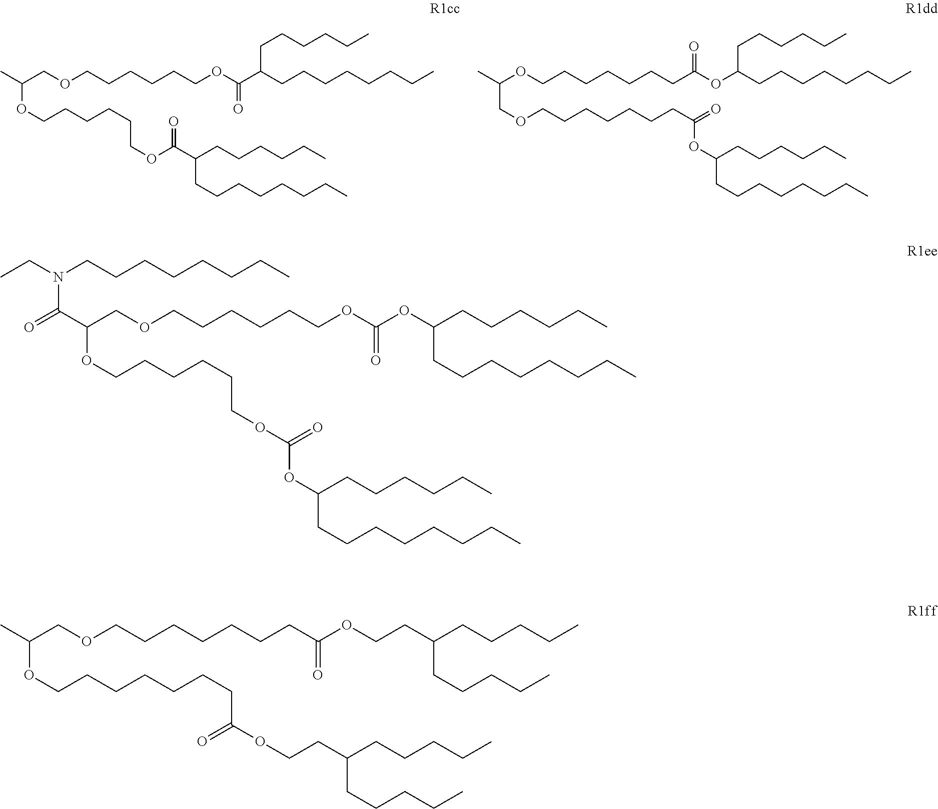

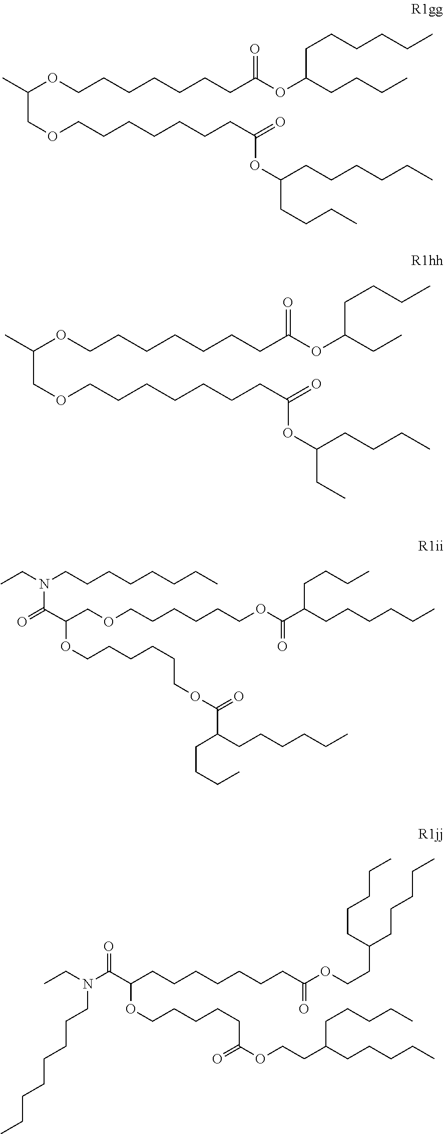

In a compound disclosed herein, Z is a spacer arm having from 2 to 24, for instance from 2 to 18, for example from 4 to 12 carbon atoms in a branched or unbranched linear saturated or unsaturated hydrocarbon chain, said chain that is interrupted by one or several atoms of oxygen, for instance Z represents a radical of formula:

—((CH₂)₂—O)ₘ—(CH₂)ᵣ(T)_{q}—*(Z);
with
* indicating the single bond linking said radical to the hydrophobic tail-group;
m being an integer from 1 to 12, for instance from 2 to 4, for example 4;
r being zero or an integer from 1 to 4;
q being zero or 1; and
T being selected in the group consisting of —(O═C)—; —(C═O)—O—**; —O—(O═C)—**; and —NH—(C═O)—O—** with ** indicating the single bond linking said group to the hydrophobic tail-group.

In a compound disclosed herein, B may be an oxygen atom.

In a compound disclosed herein, B may be a —NH— group.

In a compound disclosed herein, X may be an oxygen atom.

In a compound disclosed herein, n may be 0, 1, 2, 3 or 4.

In a compound disclosed herein, A is selected in the group consisting of —N(CH₃)₂, —N(CH₂—CH₂—CH₃)₂, O—(CH₂)₂, N(CH₃)₂, N—(CH₂)₂, N(CH₃)₂ and NR2R3-Alk-Y-group in which Y may be an oxygen or a nitrogen atom, Alk is a C₂ to C₆ alkylene and R2 and R3 are independently of each other a linear or branched (C₁-C₆) alkyl group;

In a compound disclosed herein, A is a 4- to 8-membered saturated heterocyclic radical comprising 3 to 7 carbon atoms and 1 or 2 nitrogen atoms, said 4- to 8-membered saturated heterocyclic radical being linked to the rest of the molecule by a carbon atom or a nitrogen atom and being optionally substituted by 1 to 4 substituents, independently of each other, selected from a linear or branched (C₁-C₆) alkyl group.

A compound disclosed herein may have an apparent pKa lower than 7 or ranging from 4.5 to 7.

A compound disclosed herein may be of formula (II):

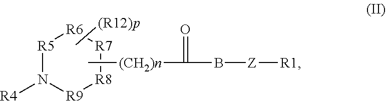

wherein
Z, n and R1 may be as defined herein;
R4 may be a (C₁-C₅) alkyl group, for instance a (C₁-C₄) alkyl group, such as a methyl group;
R12 may be a (C₁-C₅) alkyl group, for instance a (C₁-C₄) alkyl group, such as methyl group;
p may be equal to zero or 1, and for instance p may be equal to zero;
R5, R6, R7, R8 and R9 may be independently one of each other a moiety selected among —CH₂—; —CHR12— and —NH—, and the one of R5, R6, R7, R8 and R9 involved in the linkage with the rest of the molecule, being a moiety selected among —CH—; —CR12— and —N— and with the proviso that only one of R5, R6, R7, R8 and R9 is —NH— or —N—; and
B is an oxygen atom or a —NH— group, for instance an oxygen atom;
or one of its pharmaceutically acceptable salts thereof; and with said compound that is in all the possible racemic, enantiomeric and diastereoisomeric isomer forms.

A compound disclosed herein may be of formula (IIa):

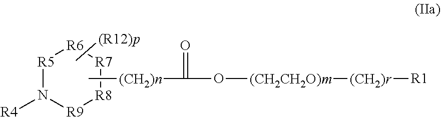

wherein
R1 may be as defined herein;
n may be 0, 1, 2, 3, 4, 5 or 6, for instance 0 to 4, such as 0, 1 or 2;
r may be 0, 1, 2, 3 or 4, for instance 0, 1 or 2;
R4 to R9, R12 and p may be as defined herein and for instance p is equal to zero;
m may be an integer from 1 to 12, for instance from 2 to 6, for example 4;
or one of its pharmaceutically acceptable salts thereof; and with said compound that is in all the possible racemic, enantiomeric and diastereoisomeric isomer forms.

A compound disclosed herein may be of formula (III):

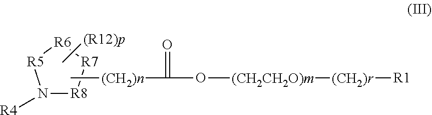

wherein
R1 may be as defined herein;
n may be 0, 1, 2, 3, 4, 5 or 6, for instance 0 to 4, such as 0, 1 or 2;
m may be an integer from 1 to 12, for instance from 2 to 6, for example 4;
r may be 0, 1, 2, 3 or 4, for instance 0, 1 or 2; and
R4 to R8, R12 and p may be as defined herein and for instance p is equal to zero;
or one of its pharmaceutically acceptable salts thereof; and with said compound that is in all the possible racemic, enantiomeric and diastereoisomeric isomer forms.

A compound disclosed herein may be of formula (IV):

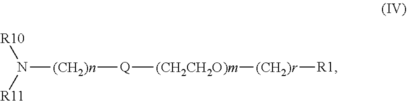

wherein
R1 may be as defined herein;
Q may be a moiety selected in the group consisting of —O(C═O)—*; —(C═O)—O*; —O(CO)O*; —N(C═O)O—* and —O(C═O)N—* with * indicating the linking to the moiety (CH₂CH₂O)ₘ.
n may be 0, 1, 2, 3, 4, 5 or 6, for instance 1 to 5, for example 2, 3 or 4;
r may be 0, 1, 2, 3 or 4, for instance 0, 1 or 2;
R10 and R11 is independently of each other a (C₁-C₅) alkyl group, for instance a (C₁-C₄) alkyl group, such as a methyl group or a propyl group; and
m may be an integer from 1 to 12, for instance from 2 to 6, and for example 4;
or one of its pharmaceutically acceptable salts thereof; and with said compound that is in all the possible racemic, enantiomeric and diastereoisomeric isomer forms.

In some embodiments, Q may be a moiety selected in the group consisting of —(C═O)—O*; —O(C═O)O*; —N(C═O)O—* and —O(C═O)N—* with * indicating the linking to the moiety (CH₂CH₂O) m.

A compound disclosed herein may be of formula (V):

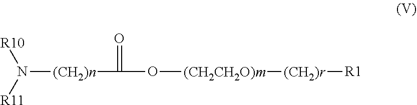

wherein
R1, R10, R11, n, m and r may be as defined herein;
or one of its pharmaceutically acceptable salts thereof; and with said compound that is in all the possible racemic, enantiomeric and diastereoisomeric isomer forms.

A compound disclosed herein may be selected in the group consisting of:

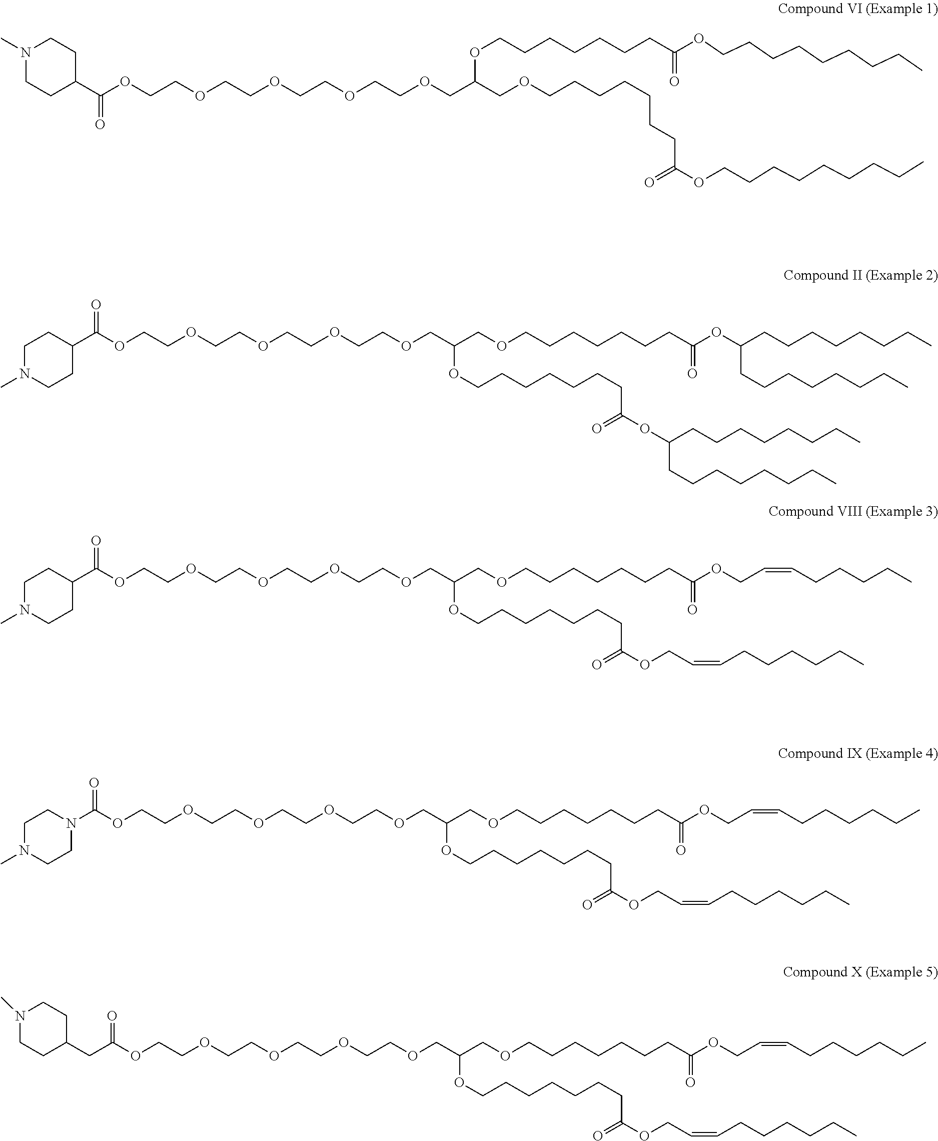

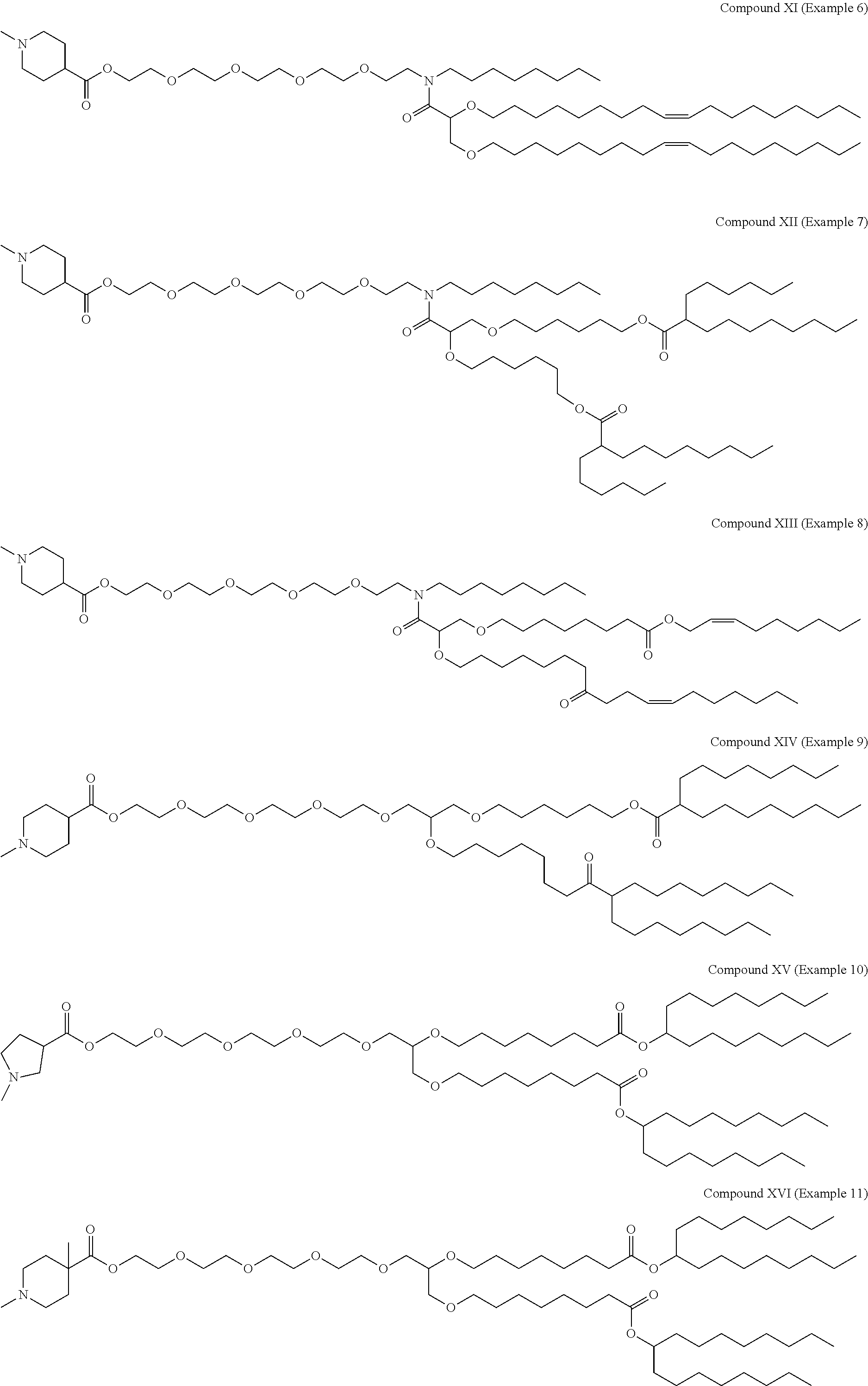

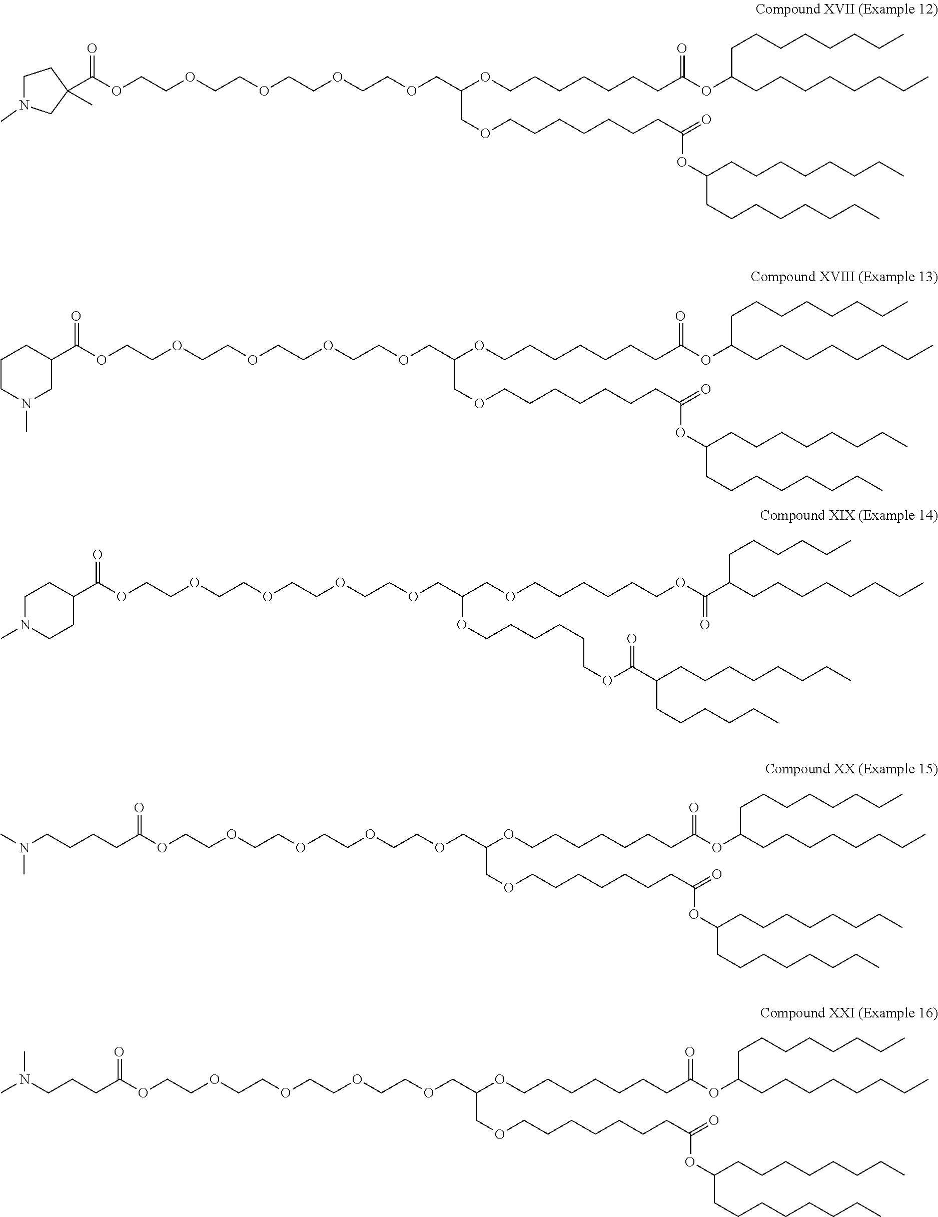

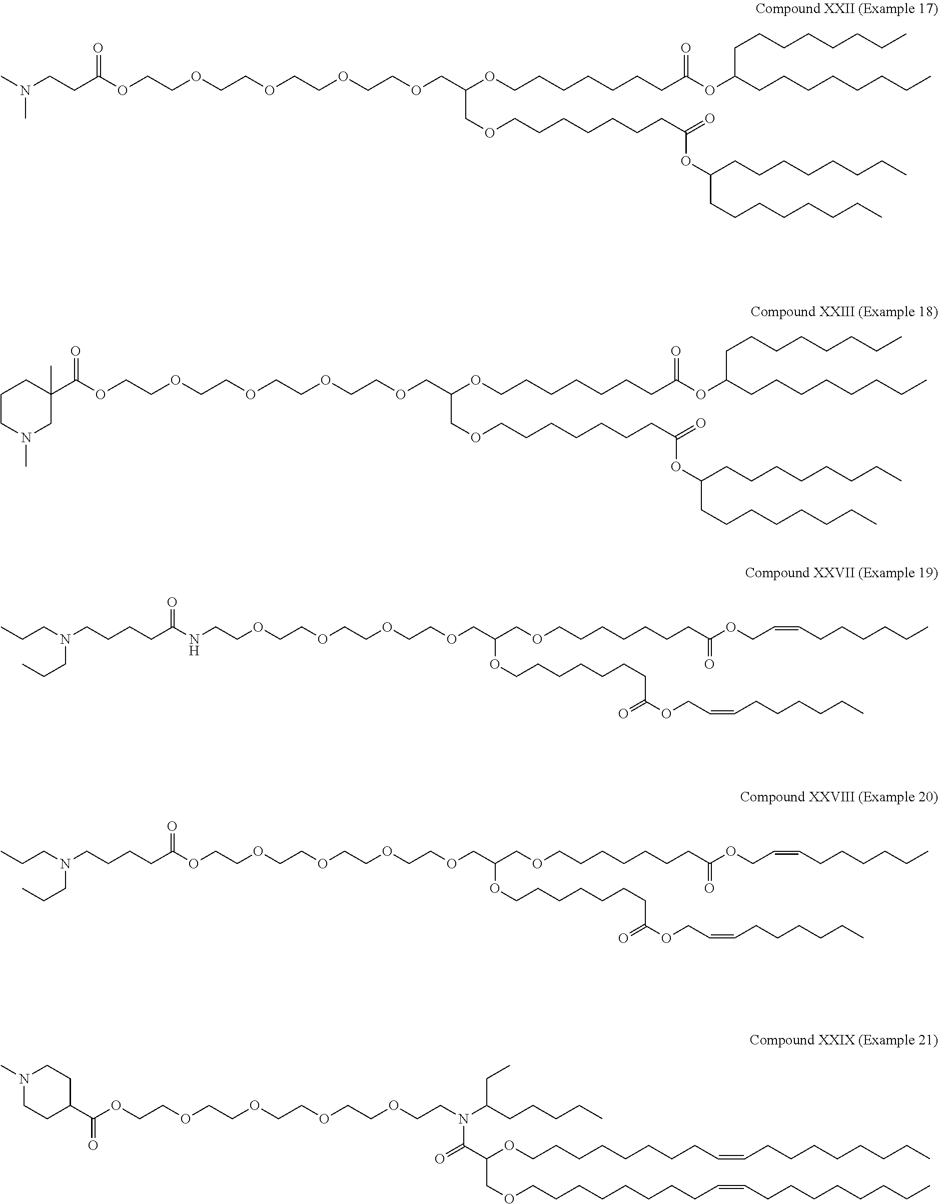

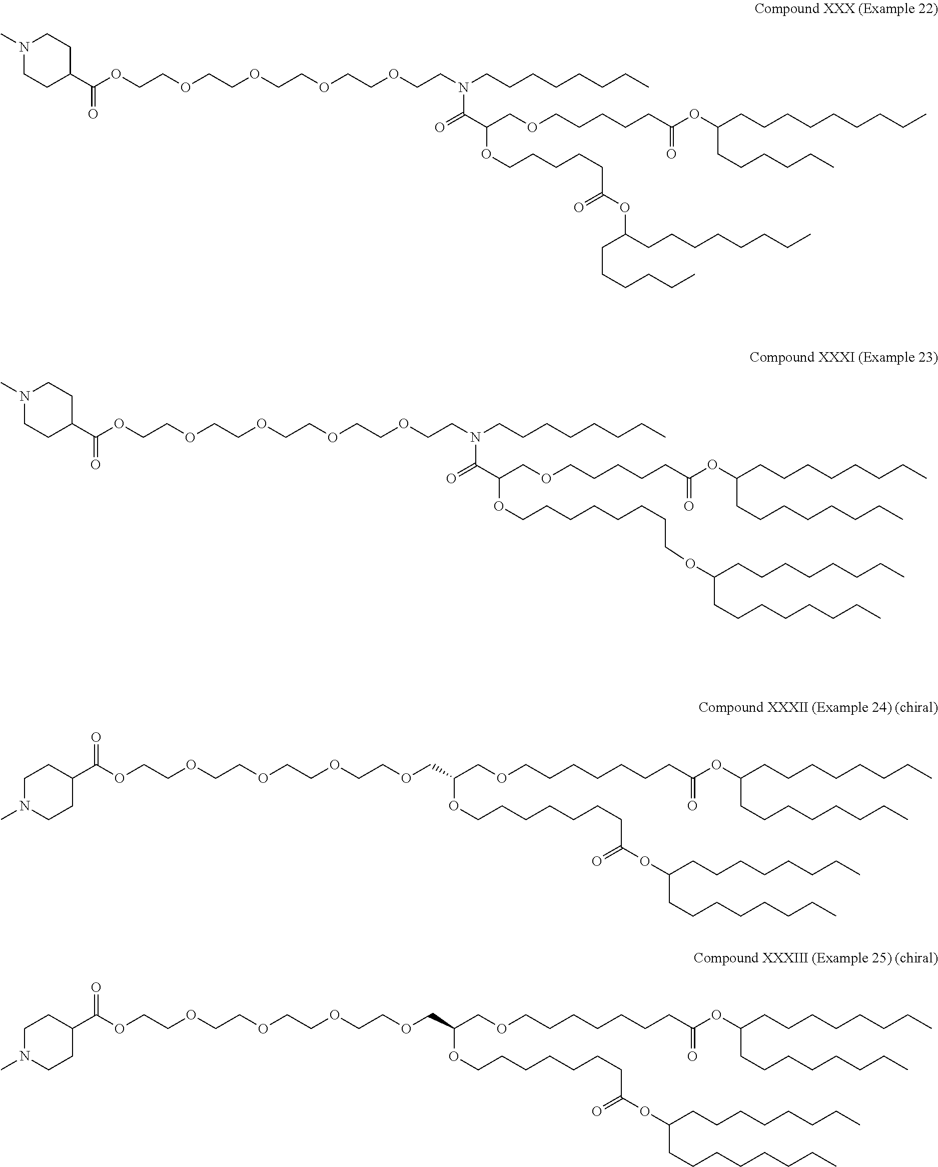

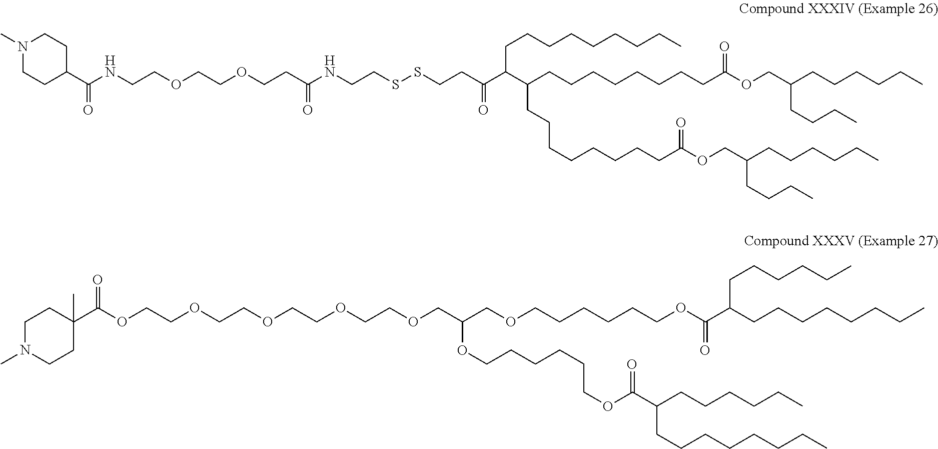

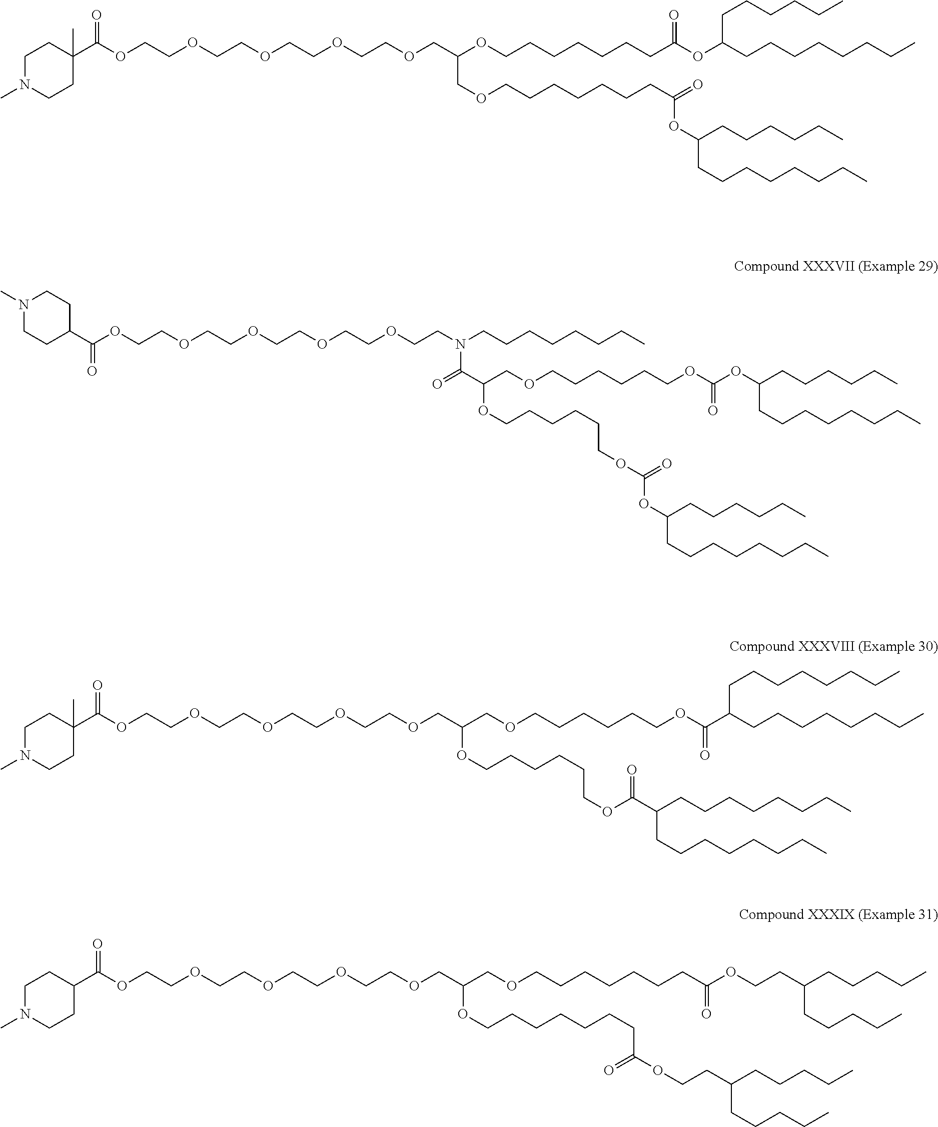

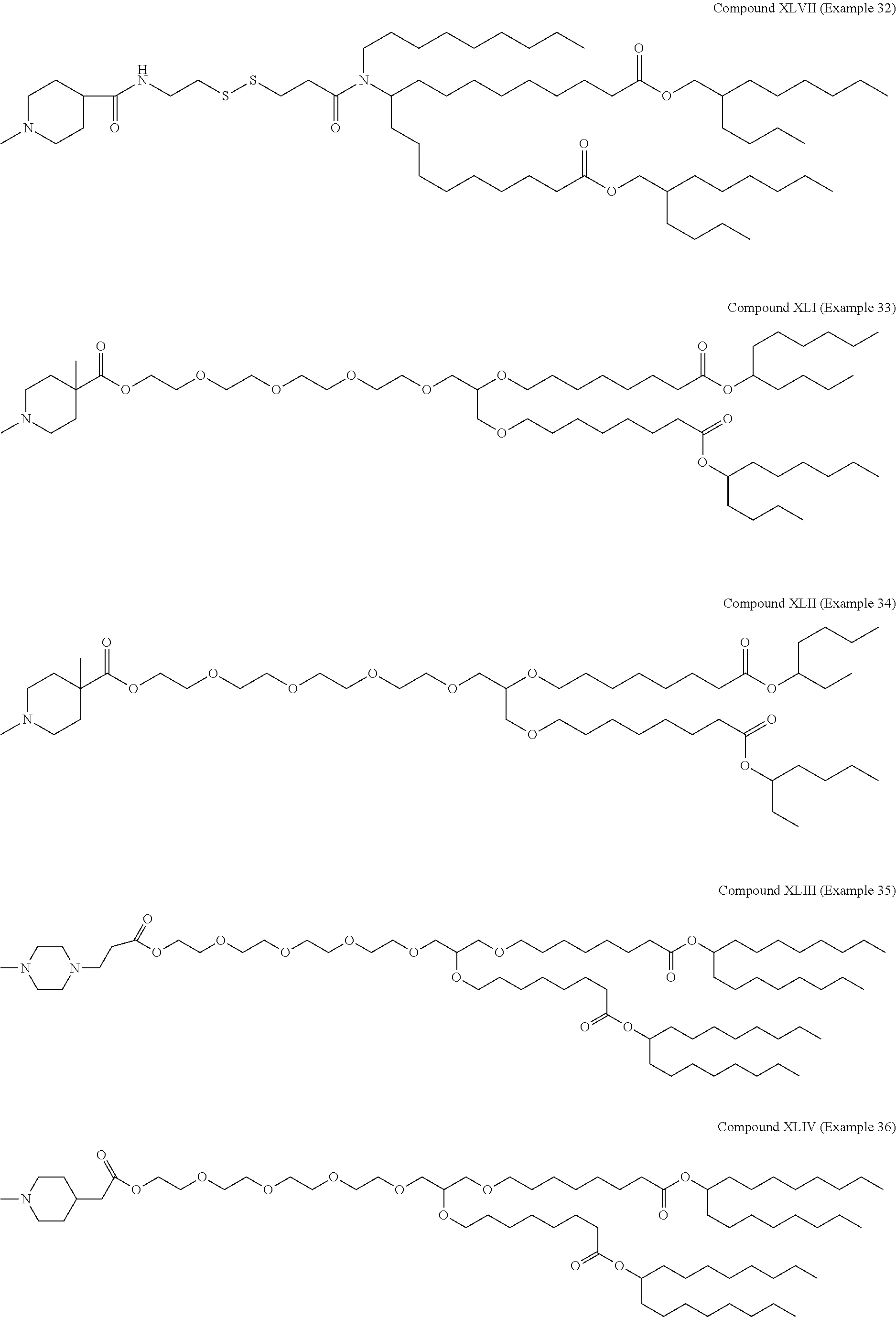

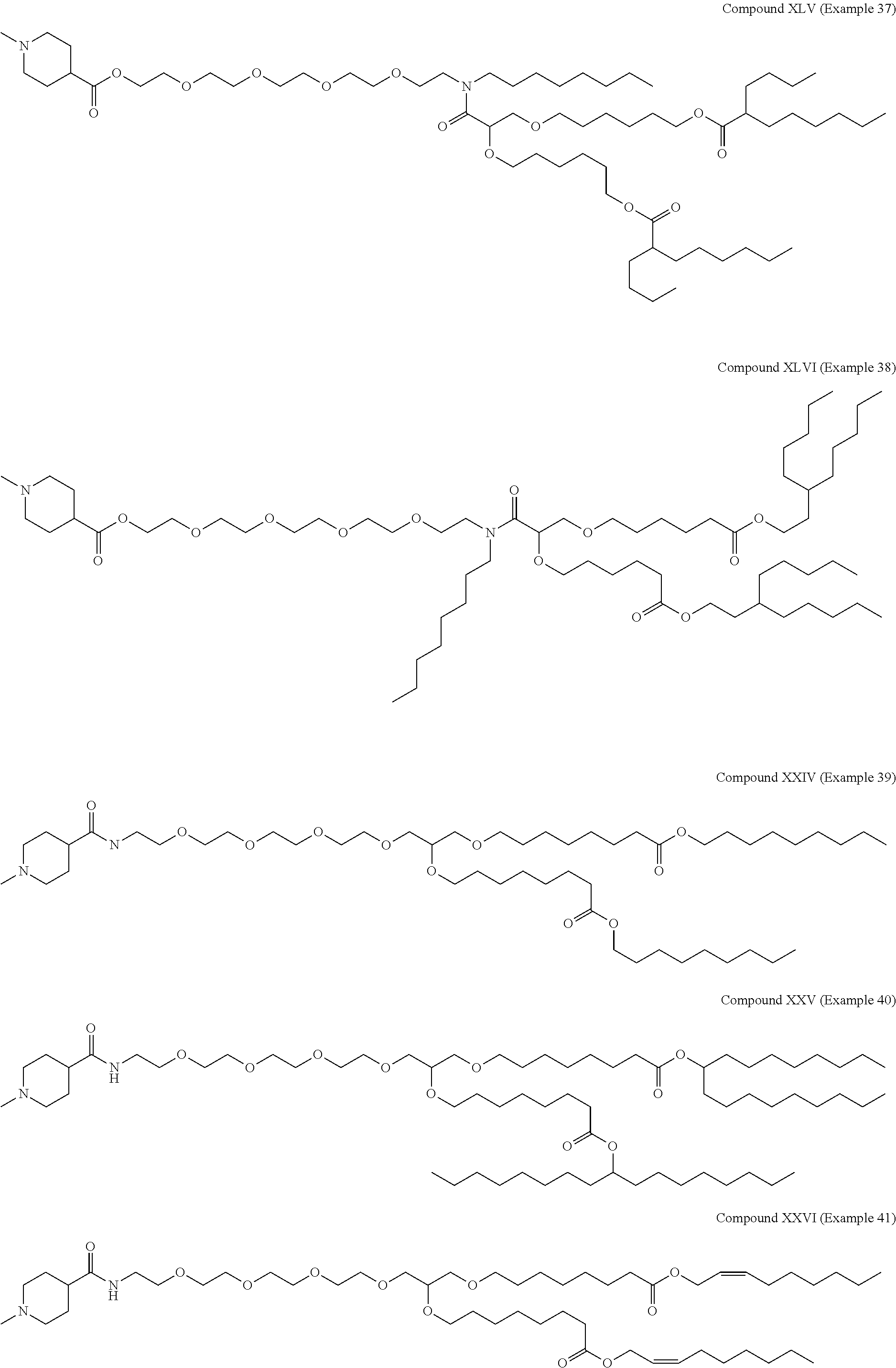

or one of its pharmaceutically acceptable salts thereof; and with said compound that is in all the possible racemic, enantiomeric and diastereoisomeric isomer forms, and in particular in the group consisting of compounds (VI), (VII), (VIII), (XI), (XII), (XIV), (XV), (XVI), (XVII), (XVIII), (XIX), (XX), (XXI), (XXX), (XXXI), (XXXII), (XXXIII), (XXXIV), (XXXV), (XXXVI), (XXXVII) or (XXXVIIII), or in the group consisting of compounds (VI), (VII), (XII), (XIV), (XVI), (XVIII), (XIX), (XXI), (XXX), (XXXI), (XXXII), (XXXIII), (XXXIV), (XXXV), (XXXVI), (XXXVII) or (XXXVIIII), or in the group consisting of compounds VII, XII, XIV, XV, XVI, XIX, XX and XXI.

In one of its objects, the disclosure relates to a composition or a lipid nanoparticle (LNP) comprising a lipid component comprising at least a lipidic compound disclosed herein.

The lipid component may further comprise at least a lipid selected from a neutral lipid, a structural lipid, and optionally a PEG-lipid.

The neutral lipid may be selected from the group consisting of phosphatidylcholines, such as DSPC, DPPC, DMPC, POPC, DOPC; phosphatidylethanolamines, such as DOPE, DPPE, DMPE, DSPE, DLPE, DEPE; DPPS; DOPG; sphingomyelins; and ceramides; and mixtures thereof.

The structural lipid may be selected from the group consisting of a sterol or an ester thereof, tomatine, alpha-tocopherol, and corticosteroid, and mixtures thereof.

The sterol or ester thereof may be selected from the group consisting of cholesterol and its derivatives, ergosterol, desmosterol, stigmasterol, lanosterol, 7-dehydrocholesterol, dihydrolanosterol, zymosterol, lathosterol, diosgenin, sitosterol, sitostanol, campesterol, fecosterol, brassicasterol, tomatidine, ursolic acid, 24-methylene cholesterol, cholesteryl margarate, cholesteryl oleate, and cholesteryl stearate, and mixtures thereof.

The PEG-lipid may be selected from the group consisting of PEG-DAG, DMG-PEG, PEG-PE, PEG-S-DAG, PEG-S-DMG, DSPC-PEG, DSPE-PEG, PEG-cer, mPEG-N,N-ditetradecylacetamide, a PEG-dialkyoxypropylcarbamate, and mixtures thereof.

A composition of a LNPs may comprise a lipidic compound disclosed herein in a molar amount of about 30% to about 70%, a neutral lipid in a molar amount of about 0% to about 50%, a structural lipid in a molar amount of about 20% to about 50%, and a PEG-lipid in a molar amount of about 1% to about 15%, in % relative to the total molar amount of the lipid component.

A composition of a LNPs disclosed herein may further comprising at least one biologically active agent.

The biologically active agent may be a nucleic acid.

The nucleic acid may encode at least an antigen.

In one of its objects, the disclosure relates to a pharmaceutical composition comprising at least a composition or a LNP disclosed herein, and a pharmaceutically acceptable excipient.

In one of its objects, the disclosure relates to an immunogenic composition comprising at least a composition or a LNP disclosed herein, for example comprising a nucleic acid encoding at least an antigen.

In one of its objects, the disclosure relates to a composition or a LNP disclosed herein, for use as a medicament.

In one of its objects, the disclosure relates to a composition or a LNP disclosed herein, for use in a method for preventing and/or treating a disease selected in a group consisting of infectious diseases, allergies, autoimmune diseases, blood disorders, metabolic diseases, neurologic diseases, and cancer diseases.

### DESCRIPTION OF THE FIGURES

FIG. 1: Schema of synthesis of compound VI (example 1);

FIG. 2: Schema of synthesis of compound VII (example 2);

FIG. 3: Schema of synthesis of compound VIII (example 3);

FIG. 4: Schema of synthesis of compound X (example 5);

FIG. 5: Schema of synthesis of compound XI (example 6);

FIG. 6: Schema of synthesis of compound XII (example 7);

FIG. 7: Schema of synthesis of compound XIII (example 8);

FIG. 8: Schema of synthesis of compound XIV (example 9);

FIG. 9: Schema of synthesis of compound XV (example 10);

FIG. 10: Schema of synthesis of compound XVI (example 11);

FIG. 11: Schema of synthesis of compound XVII (example 12);

FIG. 12: Schema of synthesis of compound XIX (example 14);

FIG. 13: Schema of synthesis of compound XX (example 15);

FIG. 14: Schema of synthesis of compound XXI (example 16);

FIG. 15: Schema of synthesis of compound XXIII (example 18);

FIG. 16: Schema of synthesis of compound XXVI (example 41);

FIG. 17: Schema of synthesis of compound XXVII (example 19);

FIG. 18: Schema of synthesis of compound XXVIII (example 20);

FIG. 19: Schema of synthesis of compound XXIX (example 21);

FIG. 20: Schema of synthesis of compound XXX (example 22);

FIG. 21: Schema of synthesis of compound XXXII (example 24);

FIG. 22: Schema of synthesis of compound XXXIII (example 25);

FIG. 23: Schema of synthesis of compound XXXIV (example 26);

FIG. 24: Schema of synthesis of compound XXXV (example 27);

FIG. 25: Schema of synthesis of compound XXXVI (example 28);

FIG. 26: Schema of synthesis of compound XXXVII (example 29);

FIG. 27: Schema of synthesis of compound XXXVIII (example 30);

FIG. 28: Schema of synthesis of compound XXXIX (example 31);

FIG. 29: Schema of synthesis of compound XLVII (example 32);

FIG. 30: Schema of synthesis of compound XLI (example 33);

FIG. 31: Schema of synthesis of compound XLII (example 34);

FIG. 32: Schema of synthesis of compound XLIII (example 35);

FIG. 33: Schema of synthesis of compound XLIV (example 36);

FIG. 34: Schema of synthesis of compound XLV (example 37);

FIG. 35: Schema of synthesis of compound XLVI (example 38);

FIG. 36: Schema of synthesis of compound XXIV (example 39);

FIG. 37: Schema of synthesis of compound XXV (example 40);

FIG. 38: Schema of synthesis of compound XXXI (example 23);

FIG. 39: represents the luminescence obtained at 24 h in Huh7 cells transfected with LNPs Lip (VII) and LNPs SS-OP containing mRNA-luc and formulated at two different molar ratios: 50/39.5/10/0.5 (−1) and 40/44.5/15/0.5 (−2) (FIG. 39A) or the cellular viability (FIG. 39B).

FIG. 40: represents the whole-body bioluminescence imaging (BLI) obtained in mice treated with LNPs SS-OP (filled square) or LNPs Lip. (VII) (filled circle) containing a luciferase encoding mRNA or PBS (filled pentagon).

FIG. 41: represents the mRNA-luc in vivo distribution of luciferase protein expression in mice treated with LNPs SS-OP (FIG. 41A), LNP Lip. (VII) (FIG. 41B) or PBS (FIG. 41C), at 6h (black bars), 24h (white bars) or 48h (grey bars) after injection.

FIG. 42: represents the mRNA-luc ex vivo tissue distribution of luciferase protein expression in mice treated with LNPs SS-OP (FIG. 42A) or LNP Lip. (VII) (FIG. 42B).

FIG. 43: represents the plasma secretion (in ng/ml) of EPO in mice measured 6 hours post-injection of LNPs Lip. (XIV), LNPs Lip. (XVII), LNPs Lip. (XVIII), LNPs Lip. (XIX), LNPs Lip. (XXX), LNPs Lip. (XXXI), LNPs Lip. (XXXII), LNPs Lip. (XXXIII), LNPs Lip. (XXXIV), LNPs Lip. (XXXV), LNPs Lip. (XXXVI), LNPs Lip. (XXXVII), LNPs Lip. (XXXVIII), and LNPs Lip. MC3 comprising, a non-replicative, highly purified, mRNA encoding the human erythropoietin.

FIG. 44: represents the HI titers in mice measured 3 weeks following a second immunization with LNPs Lip. (VII), LNPs Lip. (VIII) or LNPs Lip. (XII) comprising a natural, non-replicative mRNA encoding full-length hemagglutinin (HA) of influenza virus strain A/California/07/09 (H1N1).

### DETAILED DESCRIPTION

### Definitions

The terms used in this specification generally have their ordinary meanings in the art, within the context of this disclosure and in the specific context where each term is used. Certain terms are discussed below, or elsewhere in the specification, to provide additional guidance in describing the compositions and methods of the disclosure and how to make and use them. The following definitions are provided for the present specification, including the claims.

The term “pharmaceutically acceptable salts” includes addition salts of compounds as disclosed herein derived from the combination of such compounds with for example non-toxic acid addition salts.

The term “acid addition salts” include inorganic acids such as hydrochloric, hydrobromic, hydroiodic, sulfuric, nitric and phosphoric acid, as well as organic acids such as acetic, citric, propionic, tartaric, glutamic, salicylic, oxalic, methanesulfonic, para-toluenesulfonic, succinic, and benzoic acid, and related inorganic and organic acids.

The pharmaceutically acceptable salts of compounds as disclosed herein can also exist as various solvates, such as with water, methanol, ethanol, dimethylformamide, ethyl acetate and the like. Mixtures of such solvates can also be prepared. The source of such solvate can be from the solvent of crystallization, inherent in the solvent of preparation or crystallization, or adventitious to such solvent. Such solvates are within the scope of the present disclosure.

In the context of the present disclosure the chemical terms below have the following meanings:
a halogen atom: a fluorine, a chlorine, a bromine or an iodine;
Ct-Cz: a carbon chain that can have from t to z carbon atoms, where t and z may have the values from 1 to 7; for example, C₁-C₄ is a carbon chain that may have from 1 to 4 carbon atoms;
Alk means branched or linear alkylene C₂ to C₆;
C₁-C₄ alkyl as used herein respectively refers to C₁-C₄ normal, secondary or tertiary saturated hydrocarbon. Non limiting examples are methyl, ethyl, propyl, isopropyl, butyl, isobutyl or tertbutyl;
C₁-C₄ alkoxy is intended to mean an —O—(C₁-C₄) alkyl radical where the C₁-C₄ alkyl group is as defined above. Non limiting examples are methoxy, ethoxy, propoxy, isopropoxy, butoxy, sec-butoxy or tert-butoxy;
a heteroatom is understood to mean nitrogen, oxygen or sulphur;
a heteroaromatic ring denotes a 5- or 6-membered aromatic ring comprising 1 or 2 heteroatoms;
an aromatic ring refers to a mono or polycyclic, for example a monocyclic, aromatic hydrocarbon radical of 6-20 atoms, for example 6 atoms, derived by the removal of one hydrogen from a carbon atom of a parent aromatic ring system. An aromatic ring as disclosed herein is for example a phenyl group.

Unless otherwise defined herein, scientific and technical terms used in connection with the present disclosure shall have the meanings that are commonly understood by those of ordinary skill in the art. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, may provide one of skill with a general dictionary of many of the terms used in this disclosure. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cell and tissue culture, molecular biology, virology, immunology, microbiology, genetics, analytical chemistry, synthetic organic chemistry, medicinal and pharmaceutical chemistry, and protein and nucleic acid chemistry and hybridization described herein are those well-known and commonly used in the art. Reactions and purification techniques are performed according to manufacturer's specifications, as commonly accomplished in the art or as described herein. Further, unless otherwise required by context, singular terms shall include pluralities and plural terms shall include the singular.

Units, prefixes, and symbols are denoted in their Système International des Unités (SI) accepted form. Numeric ranges are inclusive of the numbers defining the range. Unless otherwise indicated, amino acid sequences are written left to right in amino to carboxy orientation and the nucleic acid sequences are written left to right in 5′ to 3′ direction. The headings provided herein are not limitations of the various aspects of the disclosure. Accordingly, the terms defined immediately below are more fully defined by reference to the specification in its entirety.

Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. The words “have” and “comprise,” or variations such as “has,” “having,” “comprises,” or “comprising,” will be understood to imply the inclusion of the stated element(s) (such as a composition of matter or a method step) but not the exclusion of any other elements. The term “consisting of” implies the inclusion of the stated element(s), to the exclusion of any additional elements. The term “consisting essentially of” implies the inclusion of the stated elements, and possibly other element(s) where the other element(s) do not materially affect the basic characteristic(s) of the disclosure. It is understood that the different embodiments of the disclosure using the term “comprising” or equivalent cover the embodiments where this term is replaced with “comprising only”, “consisting of” or “consisting essentially of”.

It is understood that wherever aspects are described herein with the language “comprising,” otherwise analogous aspects described in terms of “consisting of” and/or “consisting essentially of” are also provided.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “approximately” or “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). In some embodiments, the term indicates deviation from the indicated numerical value by ±10%, ±5%, ±4%, ±3%, ±2%, ±1%, ±0.9%, ±0.8%, ±0.7%, ±0.6%, ±0.5%, ±0.4%, ±0.3%, ±0.2%, ±0.1%, ±0.05%, or ±0.01%. In some embodiments, “about” indicates deviation from the indicated numerical value by ±10%. In some embodiments, “about” indicates deviation from the indicated numerical value by ±5%. In some embodiments, “about” indicates deviation from the indicated numerical value by ±4%. In some embodiments, “about” indicates deviation from the indicated numerical value by ±3%. In some embodiments, “about” indicates deviation from the indicated numerical value by ±2%. In some embodiments, “about” indicates deviation from the indicated numerical value by ±1%. In some embodiments, “about” indicates deviation from the indicated numerical value by ±0.9%. In some embodiments, “about” indicates deviation from the indicated numerical value by ±0.8%. In some embodiments, “about” indicates deviation from the indicated numerical value by ±0.7%. In some embodiments, “about” indicates deviation from the indicated numerical value by ±0.6%. In some embodiments, “about” indicates deviation from the indicated numerical value by ±0.5%. In some embodiments, “about” indicates deviation from the indicated numerical value by ±0.4%. In some embodiments, “about” indicates deviation from the indicated numerical value by ±0.3%. In some embodiments, “about” indicates deviation from the indicated numerical value by ±0.1%. In some embodiments, “about” indicates deviation from the indicated numerical value by ±0.05%. In some embodiments, “about” indicates deviation from the indicated numerical value by ±0.01%.

Within the disclosure, the terms “significantly” or “substantially” used to qualify a difference or a change, for example “significantly different of” or “substantially different from”, with respect to a feature or a parameter intends to mean that the observe change or difference is noticeable and/or it has a statistic meaning. Conversely, the terms significantly” or “substantially” used to qualify a similitude or an identity, for example “not significantly different from” or “substantially identical to”, with respect to a feature or a parameter intends to mean that change or difference are not noticeable or that any observed change or difference is not statistically different or is such that the nature and function of the concerned parameter or feature is not materially affected.

“Administer” or “administering,” as used herein refers to delivering to a subject a composition described herein, e.g., lipid nanoparticles. The composition can be administered to a subject using methods known in the art. In particular, the composition can be administered intravenously, subcutaneously, intramuscularly, intradermally, or via any mucosal surface, e.g., orally, sublingually, buccally, nasally, rectally, vaginally or via pulmonary route. In some embodiments, the administration is subcutaneous. In some embodiments, the administration is intramuscular. In some embodiments, the administration is intravenous. In some embodiments, the administration is self-administration.

The term “antigen” comprises any molecule, for example a peptide or a protein, which comprises at least one epitope that will elicit an immune response and/or against which an immune response is directed. For example, an antigen is a molecule which, optionally after processing, induces an immune response, which is for example specific for the antigen or cells expressing the antigen. After processing, an antigen may be presented by MHC molecules and reacts specifically with T lymphocytes (T cells). Thus, an antigen or fragments thereof should be recognizable by a T cell receptor and should be able to induce in the presence of appropriate co-stimulatory signals, clonal expansion of the T cell carrying the T cell receptor specifically recognizing the antigen or fragment, which results in an immune response against the antigen or cells expressing the antigen. Any suitable antigen may be envisioned which is a candidate for an immune response. An antigen may correspond to or may be derived from a naturally occurring antigen. Such naturally occurring antigens may include or may be derived from allergens, viruses, bacteria, fungi, parasites and other infectious agents and pathogens or an antigen may also be a tumor antigen. An antigen may be provided as a nucleic acid encoding antigen. After administration to an individual, the nucleic acid may be translated in a peptide or in a protein able to elicit an immune response.

The expression “charged lipid” intends to refer to any of a number of lipid species that exist in either a positively charged or negatively charged form within a useful physiological range e.g., pH ˜3 to pH ˜9. Charged lipids may be synthetic or naturally derived. Examples of charged lipids include phosphatidylserines, phosphatidic acids, phosphatidylglycerols, phosphatidylinositols, sterol hemisuccinates, dialkyl trimethylammonium-propanes, (e.g. DOTAP, DODAP, DOTMA), dialkyldimethylaminopropanes, ethyl phosphocholines, dimethylaminoethane carbamoyl sterols (e.g. DC-Choi).

As used herein, the term “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In some embodiments, the individual or subject is a human.

The expression “ionizable cationic lipid” refers to lipids containing one or more groups which can be protonated at physiological pH but may deprotonated at a pH above 8, 9, 10, 11, or 12. The ionizable cationic group may contain one or more protonatable amines which are able to form a cationic group at physiological pH. The cationic ionizable lipid compound may also further comprise one or more lipid components such as two or more fatty acids with C₆-C₂₄ alkyl or alkenyl carbon groups. These compounds may be a dendrimer, a dendron, a polymer, or a combination thereof.

The expression “lipid component” refers to a group of organic compounds that include, but are not limited to, esters of fatty acids and are generally characterized by being poorly soluble in water, but soluble in many organic solvents. Lipid is a generic term encompassing fats, fatty oils, essential oils, waxes, phospholipids, glycolipids, sulfolipids, aminolipids, chromolipids (lipochromes), and fatty acids. Within the disclosure, “lipid” encompasses neutral lipids, steroid alcohol or ester thereof, and PEGylated lipids.

The expression “lipid nanoparticles” (LNPs) refers to particles having at least one dimension on the order of nanometers (e.g., 1-1 000 nm, or for example 10-800 nm, and for example from about 80 to about 200 nm as measured by Nanoparticle Tracking Analysis (NTA)). LNPs may comprise at least one lipidic compound as disclosed herein. LNPs can further comprise a neutral lipid, a structural lipid, and/or a PEG-lipid. LNPs can be included in a formulation that can be used to deliver a biologically active agent, such as a prophylactic agent, a therapeutic agent or a diagnostic agent, to a target site of interest (e.g., cell, tissue, organ, tumor, and the like).

The expression “neutral lipid” refers to any lipid components that is either not ionizable or is a neutral zwitterionic compound at a selected pH, for example at physiological pH. Such lipids include, but are not limited to, phosphatidylcholines, phosphatidylethanolamines sphingomyelins (SM), or neutral sphingolipids such as ceramides. Neutral lipids may be synthetic or naturally derived. A neutral lipid may also be called a “helper” lipid.

Depending on context, the term “nucleotide” or “polynucleotide” may encompass a singular nucleic acid as well as plural nucleic acids. Within the disclosure the term “nucleic acid”, “polynucleotide”, and “oligonucleotides” are used interchangeably.

They refer to a polymeric form of at least two nucleotides, either deoxyribonucleotides or ribonucleotides, or analogs thereof. Nucleic acids may have any three-dimensional structure, and may perform any function, known or unknown. In some embodiments, a polynucleotide is an isolated nucleic acid molecule or construct, e.g., messenger RNA (mRNA) or plasmid DNA (pDNA). In some embodiments, a polynucleotide comprises a conventional phosphodiester bond. In some embodiments, a polynucleotide comprises a non-conventional bond (e.g., an amide bond, such as found in peptide nucleic acids (PNA)). The term “nucleic acid” may refer to any one or more nucleic acid segments, e.g., DNA or RNA fragments, present in a polynucleotide. By “isolated” nucleic acid or polynucleotide is intended a nucleic acid molecule, DNA or RNA, which has been removed from its native environment. Further examples of an isolated polynucleotide include recombinant polynucleotides maintained in heterologous host cells or purified (partially or substantially) from other polynucleotides in a solution. Isolated RNA molecules include in vivo or in vitro RNA transcripts of polynucleotides of the present disclosure. Isolated polynucleotides or nucleic acids according to the present disclosure further include such molecules produced synthetically. In addition, a polynucleotide or a nucleic acid can include regulatory elements such as promoters, enhancers, ribosome binding sites, or transcription termination signals. “Nucleic acid”, “polynucleotide”, and “oligonucleotides” may be linear or cyclic. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, closed-ended DNA (ceDNA), self-amplifying RNA (saRNA), stranded DNA (ssDNA), small interfering RNA (siRNA) and micro RNA (miRNA), recombinant polynucleotides, branched polynucleotides, plasmids, vectors, isolated DNA of any sequence, isolated RNA of any sequence, nucleic acid probes, and primers. A nucleic acid may comprise modified nucleotides, such as methylated nucleotides and nucleotide analogs. If present, modifications to the nucleotide structure may be imparted before or after assembly of the polymer. The sequence of nucleic acids may be interrupted by non-nucleotide components. A nucleic acid may be further modified after polymerization, such as by conjugation with a labeling component. The term “complement of a nucleic acid” denotes a nucleic acid molecule having a complementary base sequence and reverse orientation as compared to a reference sequence, such that it could hybridize with a reference sequence with complete fidelity. “Recombinant” as applied to a nucleic acid means that the nucleic acid is the product of various combinations of in vitro cloning, restriction and/or ligation steps, and other procedures that result in a construct that can potentially be expressed in a host cell.

The expressions “PEG-lipid” or “PEGylated lipid” are used interchangeably and intend to refer to a molecule comprising both a lipid portion and a polyethylene glycol portion. PEG-lipid are known in the art and include 1-(monomethoxy-polyethyleneglycol)-2,3-dimyristoylglycerol (PEG-DMG), and the like.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced recombinant technology but is not necessarily translated from a designated nucleic acid sequence. It can be generated in any manner, including by chemical synthesis. An “isolated” polypeptide or a fragment, variant, or derivative thereof refers to a polypeptide that is not in its natural environment. No particular level of purification is required. For example, an isolated polypeptide can simply be removed from its native or natural environment. Recombinantly produced polypeptides and proteins expressed in host cells are considered isolated for the purpose of the disclosure, as are native or recombinant polypeptides which have been separated, fractionated, or partially or substantially purified by any suitable technique.

The expressions “sterol” or “steroid alcohol” are used interchangeably and intend to refer to a group of lipids comprised of a sterane core bearing a hydroxyl moiety. As example of steroid alcohol, one may cite cholesterol, campesterol, sitosterol, stigmasterol and ergosterol. Esters of steroid alcohol or of sterol refer to ester of carboxylic acid with the hydroxyl group of the steroid alcohol. Suitable carboxylic acid comprises, further to the carboxyl moiety, a saturated or unsaturated, linear or branched, alkyl group. In some embodiments the alkyl group may be a C₁-C₂₀ alkyl group. In other embodiments, the carboxylic acid may be a fatty acid.

As used herein, the terms “prevent”, “preventing” or “delay progression of” (and grammatical variants thereof) with respect to a disease or disorder relate to prophylactic treatment of a disease, e.g., in an individual suspected to have the disease, or at risk for developing the disease. Prevention may include, but is not limited to, preventing or delaying onset or progression of the disease and/or maintaining at least one symptom of the disease at a desired or sub-pathological level. The term “prevent” does not require the 100% elimination of the possibility or likelihood of occurrence of the event. Rather, it denotes that the likelihood of the occurrence of the event has been reduced in the presence of a composition or method as described herein.

The terms “treat” or “treatment” or “therapy” in the present text refers to the administration or consumption of a composition as disclosed herein with the purpose to cure, heal, alleviate, relieve, alter, remedy, ameliorate, improve, or affect a disorder, the symptoms of the condition, or to prevent or delay the onset of the symptoms, complications, or otherwise arrest or inhibit further development of the disorder in a statistically significant manner.

As used herein, the terms “therapeutically effective amount” and “prophylactically effective amount” refer to an amount that provides a therapeutic benefit in the treatment, prevention, or management of pathological processes considered. The specific amount that is therapeutically effective can be readily determined by an ordinary medical practitioner and may vary depending on factors such as the type and stage of pathological processes considered, the patient's medical history and age, and the administration of other therapeutic agents.

As used herein, “target cells” or “targeted cells” refer to cells of interest. The cells may be found in vitro, in vivo, in situ or in the tissue or organ of an organism. The organism may be an animal, for example a mammal, for example a human, and for example a human patient. In some embodiments, a target cell is a cell isolated from an individual.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure. All publications mentioned herein are incorporated herein by reference to disclose and describe the methods and/or materials in connection with which the publications are cited.

The list of sources, ingredients, and components as described hereinafter are listed such that combinations and mixtures thereof are also contemplated and within the scope herein.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All lists of items, such as, for example, lists of ingredients, are intended to and should be interpreted as Markush groups. Thus, all lists can be read and interpreted as items “selected from the group consisting of” the list of items “and combinations and mixtures thereof.”

Referenced herein may be trade names for components including various ingredients utilized in the present disclosure. The inventors herein do not intend to be limited by materials under any particular tradename. Equivalent materials (e.g., those obtained from a different source under a different name or reference number) to those referenced by tradename may be substituted and utilized in the descriptions herein.

All publications and other references mentioned herein are incorporated by reference in their entirety. Although a number of documents are cited herein, this citation does not constitute an admission that any of these documents forms part of the common general knowledge in the art.

### Lipidic Compounds

The lipidic compounds disclosed herein are ionizable cationic lipidic compounds.

Lipidic compounds as disclosed herein are for example ionizable since they are amine-containing lipidic compounds. As such compounds can be readily protonated, their pKa change according to the value of pH. For example, the compounds as disclosed herein have for example a pKa lower than 7 and for example ranging from 4.5 to 6.7.

The lipidic compounds as disclosed herein may have asymmetric centers, chiral axes, and chiral planes (as described in: E. L. Eliel and S. H. Wilen, Stereochemistry of Carbon Compounds, John Wiley & Sons, New York, 1994, pages 1119-1190), and occur as racemates, racemic mixtures, and as individual diastereomers, with all possible isomers and mixtures thereof, including optical isomers, being included in the present disclosure. In addition, the cationic lipids disclosed herein may exist as tautomers and both tautomeric forms are intended to be encompassed by the scope of the disclosure, even though only one tautomeric structure is depicted.

The pharmaceutically acceptable salts of compounds as disclosed herein have one or several counter ions which are generally physiologically acceptable. As possible counter ions may be for example cited halide, phosphate, trifluroroacetate, sulfite, nitrate, gluconate, glucuronate, galacturonic acid radical, alkylsulfonate, alkylcarboxylate, propionic sulfonate and methanesulfonic acid radical.

The compounds as disclosed herein and the pharmaceutically acceptable salts thereof can also exist as various solvates, such as with water, methanol, ethanol, dimethylformamide, ethyl acetate and the like. Mixtures of such solvates can also be prepared. The source of such solvate can be from the solvent of crystallization, inherent in the solvent of preparation or crystallization, or adventitious to such solvent. Such solvates are within the scope of the present disclosure.

For example, the lipidic compounds as disclosed herein have formula (I):

A—(CH₂)ₙ—CX—B—Z—R1 (I)
wherein:
   R1 is a C₁₀ to C₅₇, or C₁₀ to C₅₅ lipophilic or hydrophobic tail-group;
   Z is a spacer arm having from 2 to 24, for instance from 2 to 18, for example from 4 to 12 carbon atoms in a branched or unbranched linear saturated or unsaturated hydrocarbon chain, said chain that is interrupted by one or several atoms of oxygen and/or moieties selected among —S—S—; —(O═C)—; —(C═O)—O—; —O—(O═C)—; —S—; —NH—, —NH—(O═C)—; —(O═C)—NH— and —NH—(C═O)—O— and for instance by —(C═O)—O—; —O—(O═C)— and —NH—(C═O)—O— and optionally having an oxygen atom or a moiety selected among —NH—(O═C)—*—O—(O═C)—*; —(C═O)—O—*; and —(O═C)— to its end linked to the hydrophobic tail-group, with * indicating the single bond linking said moiety to the hydrophobic tail-group;
   B represents an oxygen atom or a —NH— group;
   X is an oxygen atom or a sulfur atom;
   n is 0, 1, 2, 3, 4, 5 or 6; and
   A represents a group selected in the group consisting of:
   a R2R3N-group in which R2 and R3 represent independently of each other a linear or branched (C₁-C₆) alkyl group,
   a NR2R3-Alk-Y-group in which Y is an oxygen or a nitrogen atom, Alk is an alkylene moiety in C₂ to C₆ and R2 and R3 represent independently of each other a linear or branched (C₁-C₆) alkyl group,
   a 4- to 8-membered saturated heterocyclic radical comprising 3 to 7 carbon atoms and 1 or 2 nitrogen atoms, said 4- to 8-membered saturated heterocyclic radical being linked to the rest of the molecule by a carbon atom or a nitrogen atom and being optionally substituted by 1 to 4 substituents, independently of each other, selected from a linear or branched (C₁-C₆) alkyl group;
or one of its pharmaceutically acceptable salts thereof; and with said compound that is in all the possible racemic, enantiomeric and diastereoisomeric isomer forms.

According to one embodiment, the compounds as disclosed herein are under a cationic form.

According to one embodiment, B is an oxygen atom.

According to one embodiment, B is a —NH— group.

According to one embodiment, X is an oxygen atom.

According to one embodiment, n is 0, 1, 2, 3 or 4.

In some embodiments, R1 is a C₁₀ to C₅₅ lipophilic or hydrophobic tail-group.

According to one specific embodiment, R1 is an optionally substituted, branched or unbranched linear, saturated or unsaturated, C₁₀ to C₅₇, or C₁₀ to C₅₅, and for example C₁₀ to C₅₅, hydrocarbon radical, and which hydrocarbon skeleton that is optionally interrupted by one or several atoms of oxygen or nitrogen and/or one or several moiety —(O═C)—; —(C═O)—O—; —O—(O═C)— and which one nitrogen atom, if present in the skeleton, can be linked, directly or not, to said Z radical.

For example, the hydrophobic or lipophilic tail, R1, may comprise at least two, three or more hydrocarbon chains each one independently being selected from optionally substituted C₈-C₂₄, for example C₁₀-C₂₀, alkyl chain, optionally substituted variably saturated or unsaturated C₈-C₂₄, for example C₁₀-C₂₀, alkenyl chain and optionally substituted saturated, variably saturated or unsaturated C₈-C₂₄, for example C₁₀-C₂₀, acyl chain with said alkyl, alkenyl or acyl chains can be interrupted by one or several atoms of oxygen or nitrogen and/or one or several moieties like —(C═O)—O—; —O—(O═C)— and preferably by at least one moiety like —(C═O)—O—; —O—(O—C)—.

Each hydrocarbon chain may be substituted by at least one radical selected from —OH, and —CO₂H.

According to one specific embodiment, the hydrophobic or lipophilic tail is selected in the group consisting of:

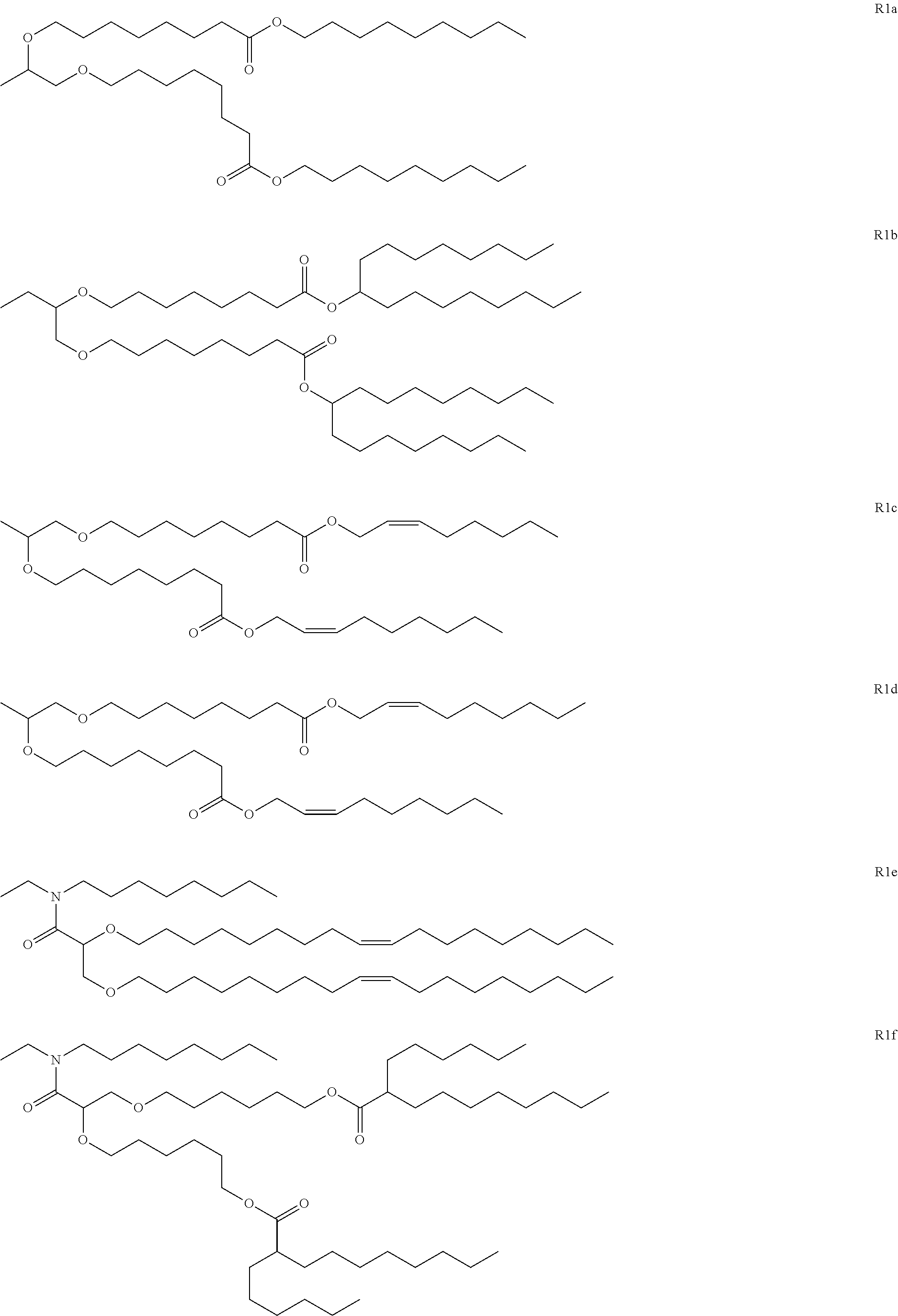

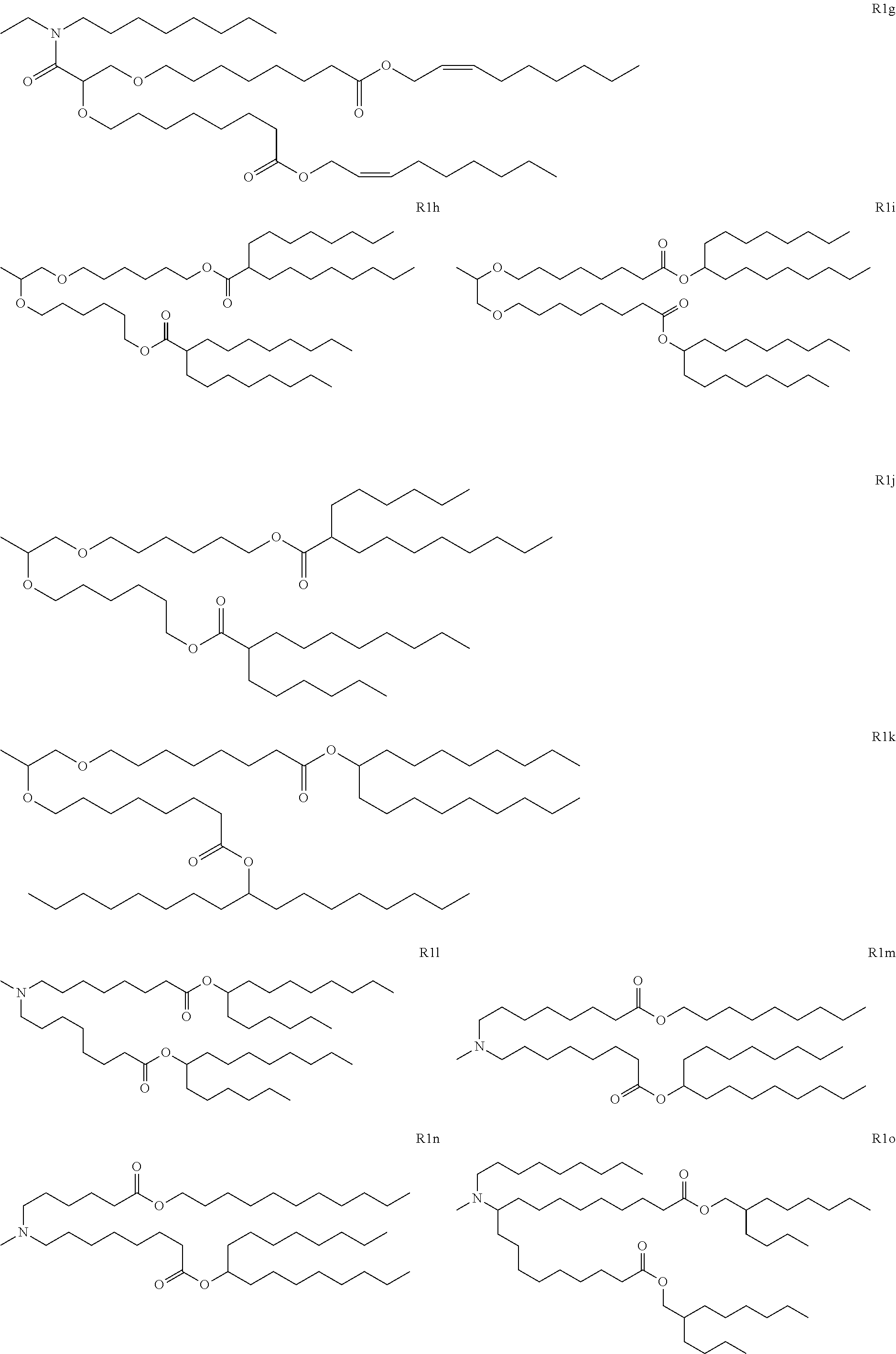

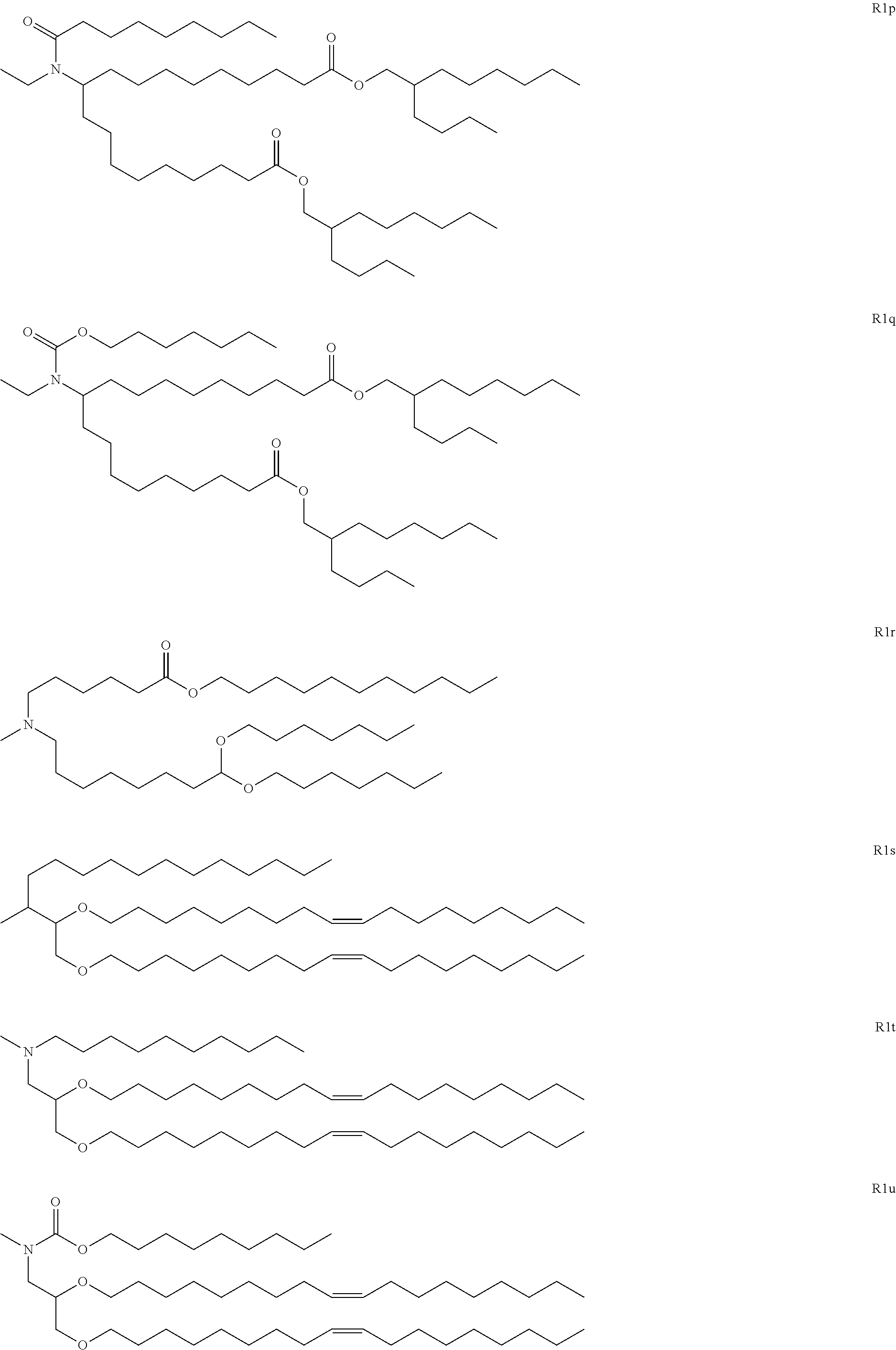

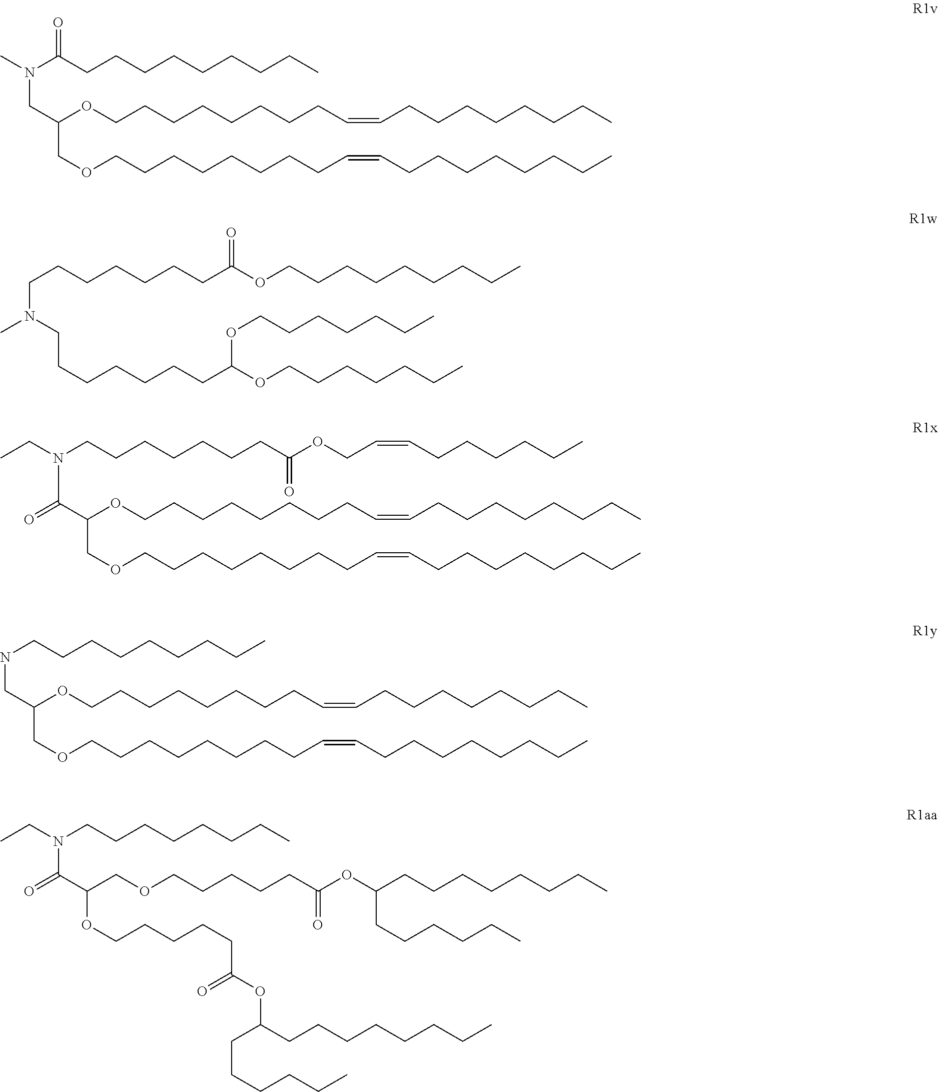

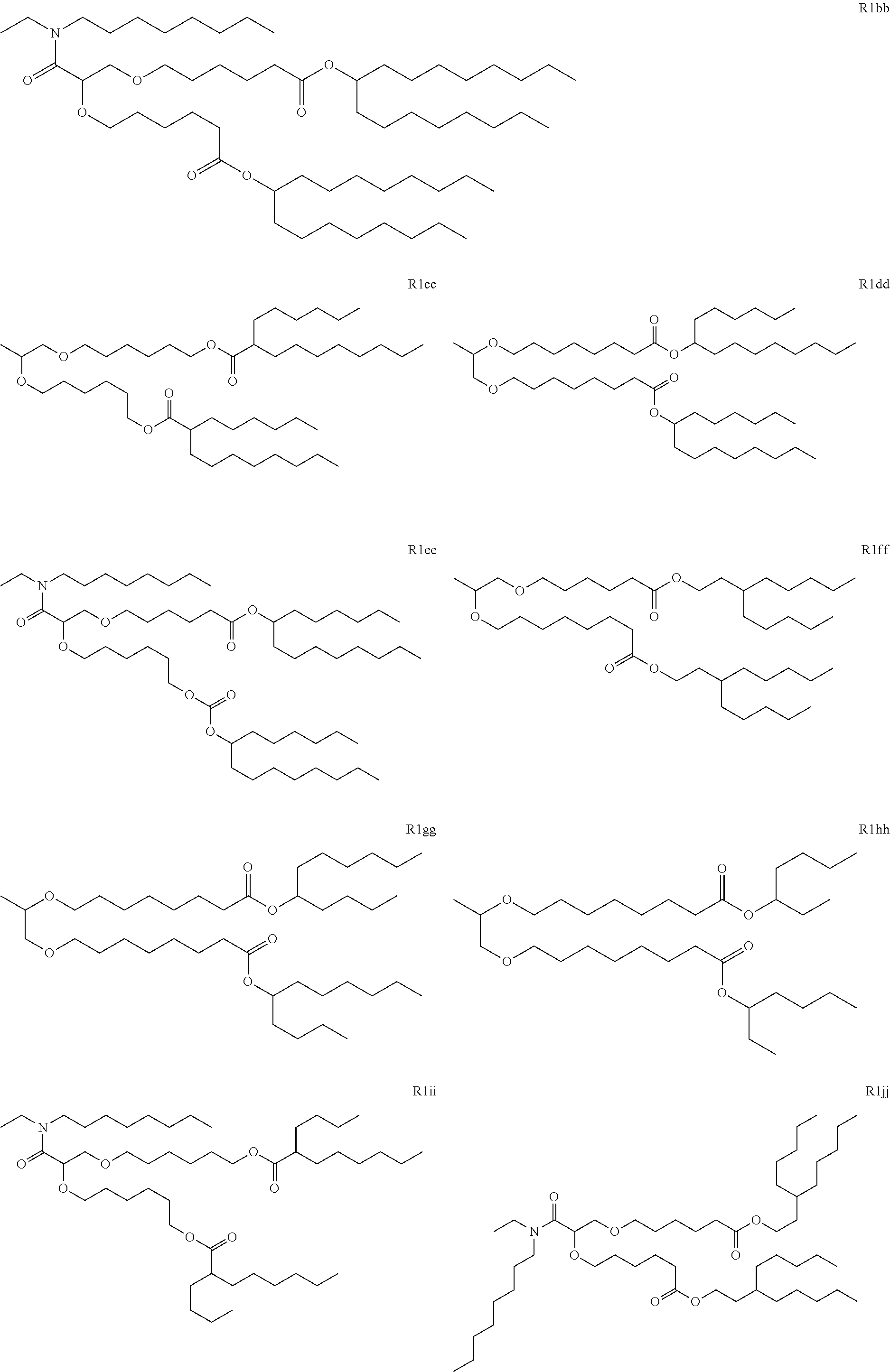

In one embodiment, the hydrophobic or lipophilic tail of compounds according to the disclosure do not include any nitrogen atom. In this specific embodiment, the hydrophobic or lipophilic tail is in particular selected among R1a, b, c, d, h, i, j, k, s, cc, dd, ff, gg and hh.

In another embodiment, the hydrophobic or lipophilic tail of compounds according to the disclosure contains one nitrogen atom and this one is directly or not linked to Z. In this specific embodiment, the hydrophobic or lipophilic tail is in particular selected among R1e, f, g, l, m, n, o, p, q, r, t, u, v, w, x y, z, aa, bb, ee, ii, and jj.

In another embodiment, the hydrophobic or lipophilic tail of compounds according to the disclosure contains at least three or more hydrocarbon chains like for example in the hydrophobic or lipophilic tails R1b, e, f, g, h, i, j, k, I, m, n, o, p, q r, s, t, u, v, w, x, y, z, aa, bb, cc, dd, ee, ff, gg, hh, ii and j. Each hydrocarbon chain may be selected among substituted C₈-C₂₄, for example C₁₀-C₂₀, alkyl chain and substituted variably saturated or unsaturated C₈-C₂₄, for example C₁₀-C₂₀, alkenyl chain and with said alkyl or alkenyl chains optionally and preferably being interrupted by one or several moieties like —(C═O)—, —O—(C═O)—** or —(C═O)—O—**.

In a specific embodiment, the hydrophobic or lipophilic tail of compounds according to the disclosure is the tail selected among R1b, R1f, R1h, R1i, R1j, R1bb, R1hh, R1ii, and in particular R1i.

According to another embodiment, the cationic and/or ionizable lipidic compounds as disclosed herein have a Z radical of formula

—((CH₂)₂—O)ₘ—(CH₂)ᵣ(T)_{q}—*(Z)
with
   * indicating the single bond linking said radical to the hydrophobic tail-group,
   m being an integer from 1 to 12, for instance from 2 to 4, for example 4,
   r being zero or an integer from 1 to 4,
   q being zero or 1 and
   T being selected in the group consisting of —(O═C)—; —(C═O)—O—**; —O—(O═C)—**; and —NH—(C═O)—O—** with ** indicating the single bond linking said group to the hydrophobic tail-group.

In a specific embodiment, q and r are different from zero and for instance, r is equal to 1 or 2. For example, Z is Za, Zb, Zc, or Zd:

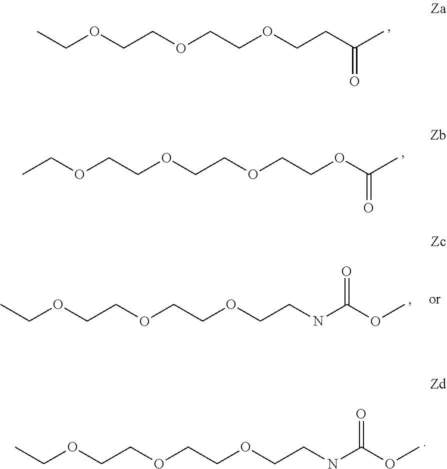

In another specific embodiment, q is equal to 1 and r is equal to 0. For example, Z is Ze or Zf:

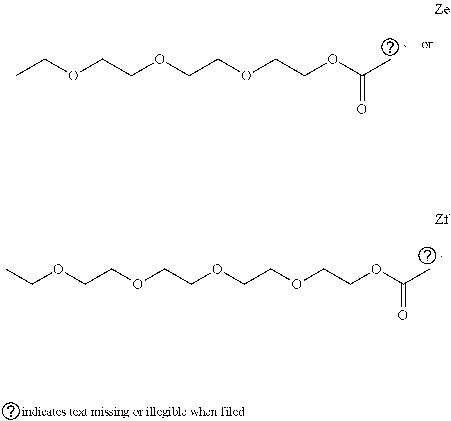

In another specific embodiment, q is equal to zero and r is different from zero and for example r is equal to 1 or 2. For example, Z is Zg or Zh:

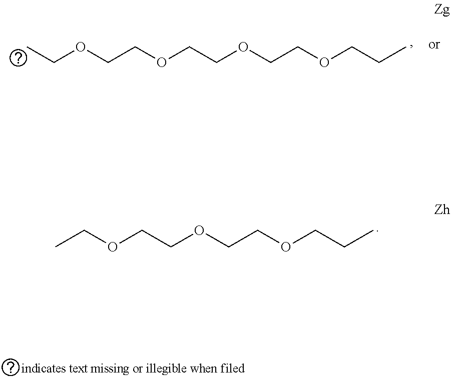

In another specific embodiment, r and q are equal to zero and Z is —((CH₂)₂—O)ₘ— with m as defined here-above.

In another specific embodiment, Z is

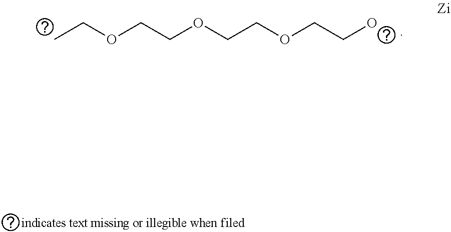

In another specific embodiment, Z is

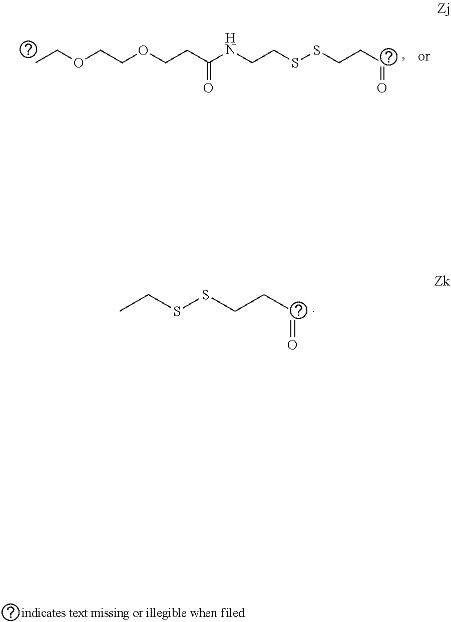

In a specific embodiment, Z is selected among Za, b, c, d, f, g, h, I, j and k.

Regarding, the radical A, it represents, in a specific embodiment, a R2R3N-group in which R2 and R3 represent independently of each other a linear or branched (C₁-C₆) alkyl group. For example, A is —N(CH₃)₂, —N(CH₂—CH₂—CH₃).

In a specific embodiment, the compounds of formula (I) comprise one R1—Z— moiety selected among the following ones:

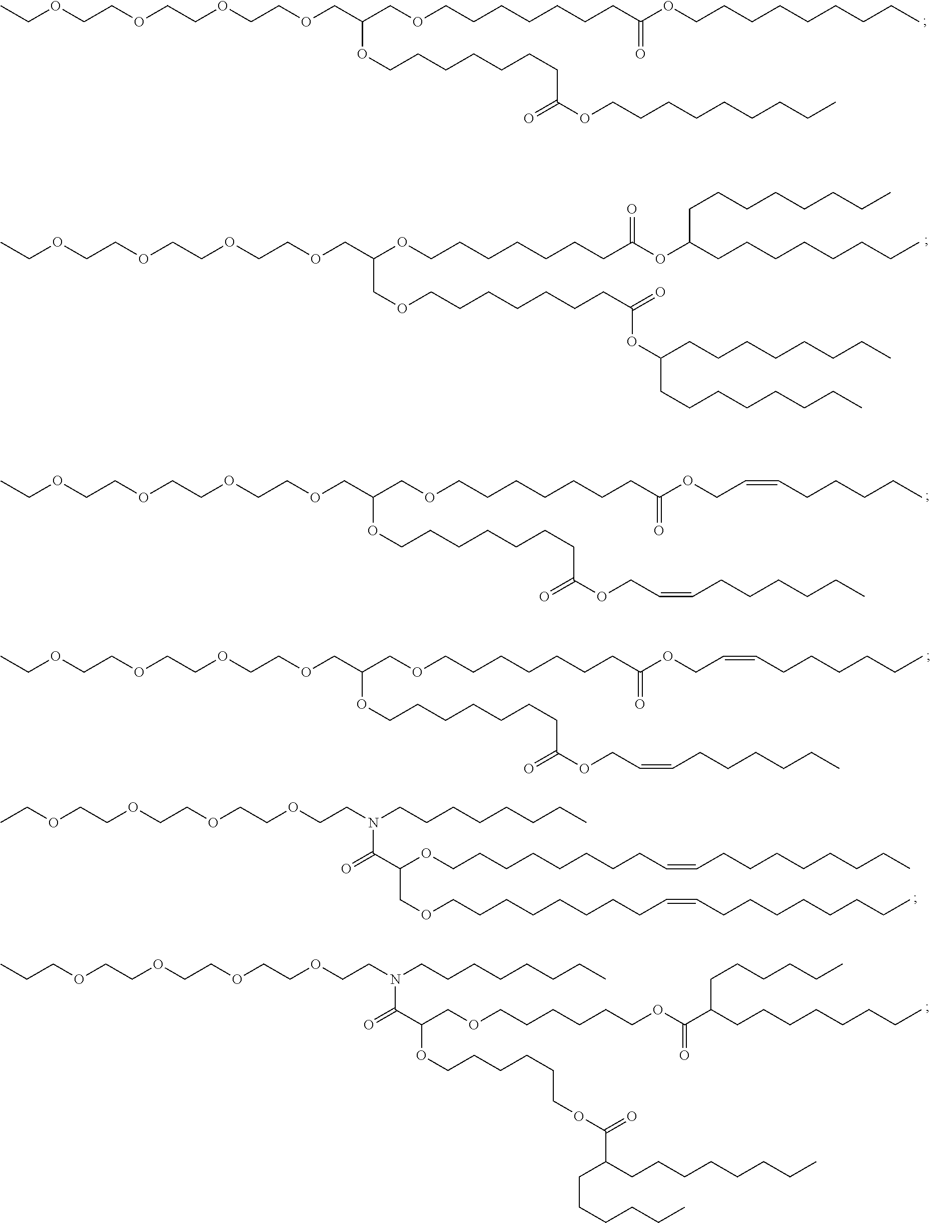

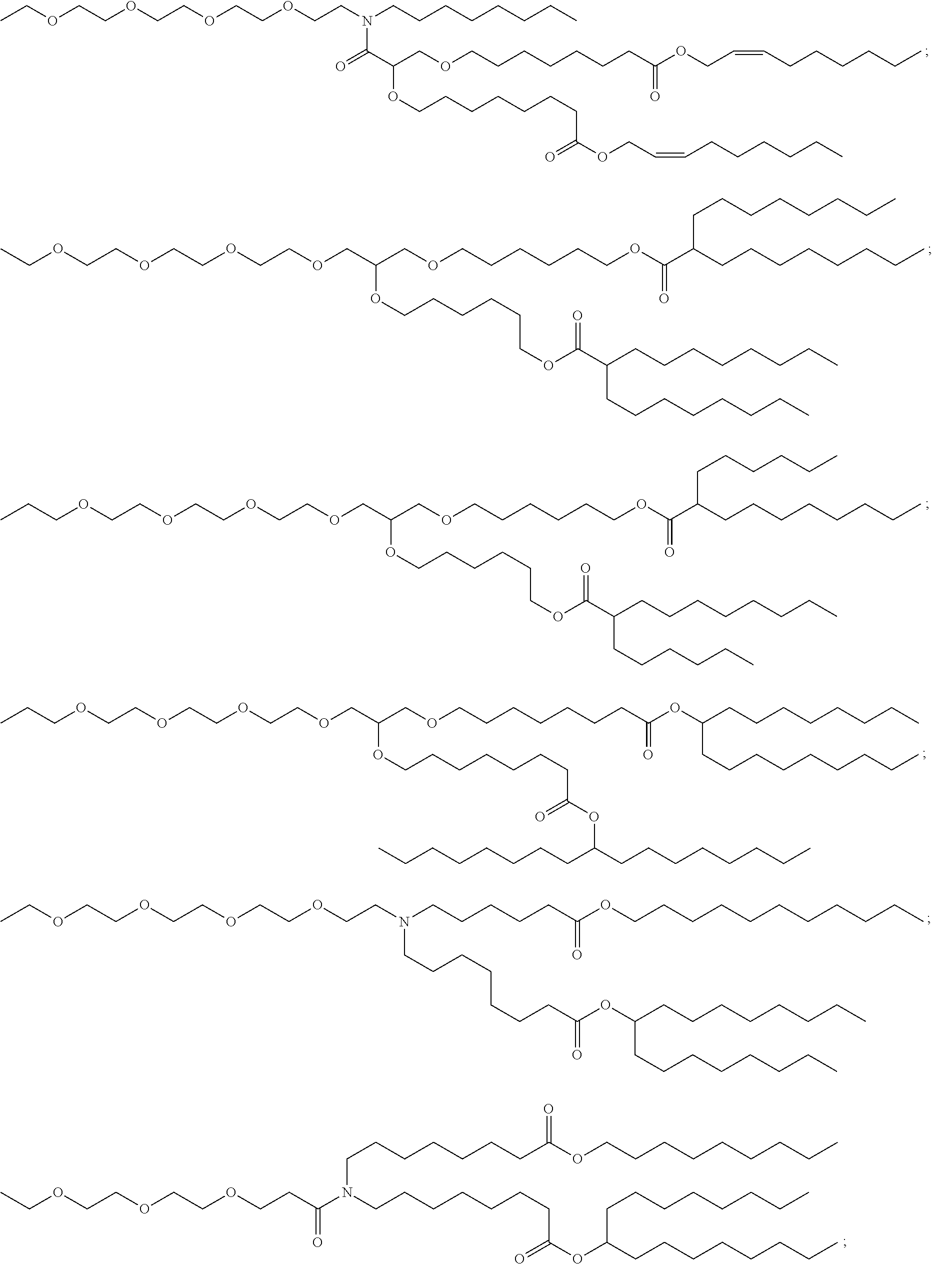

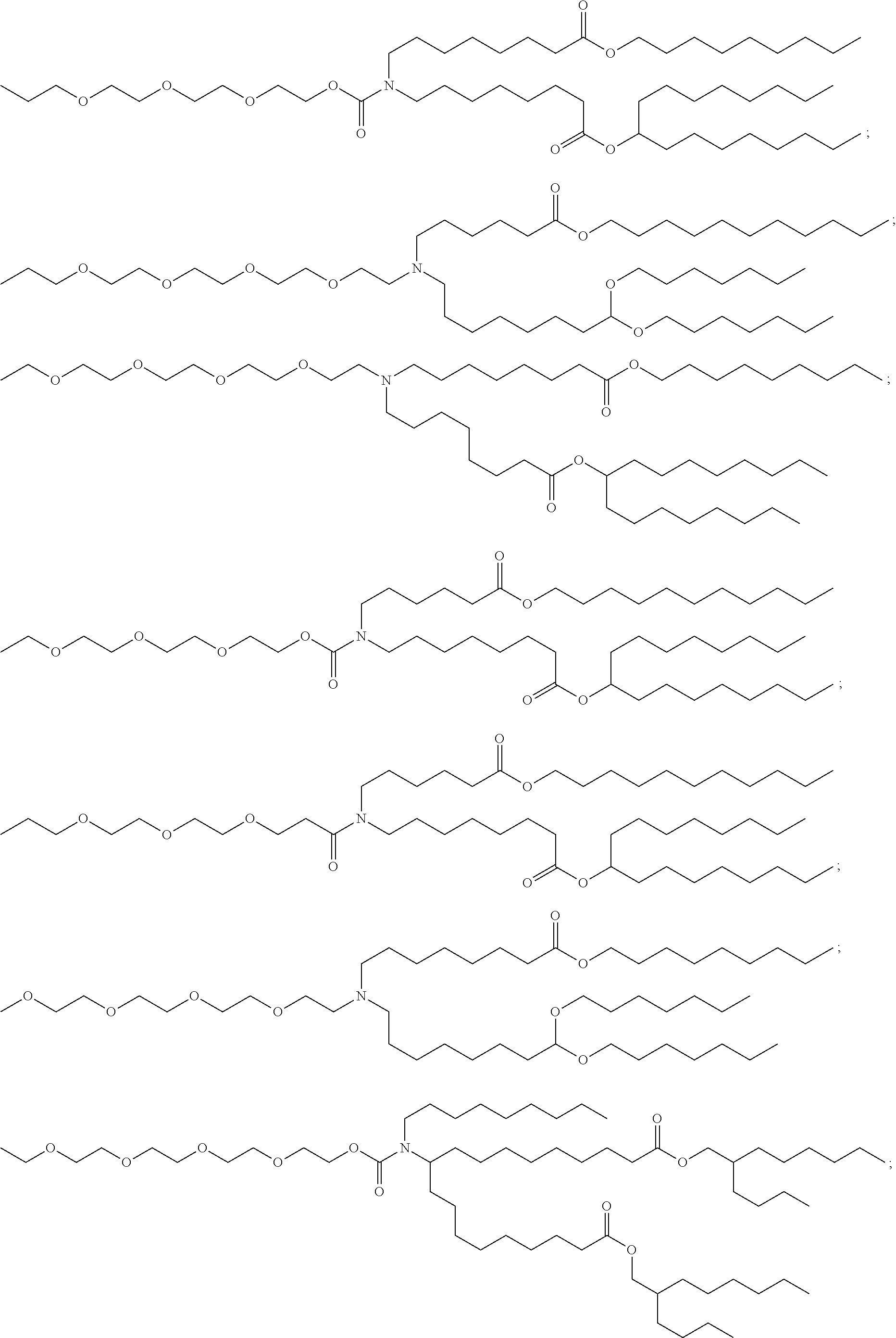

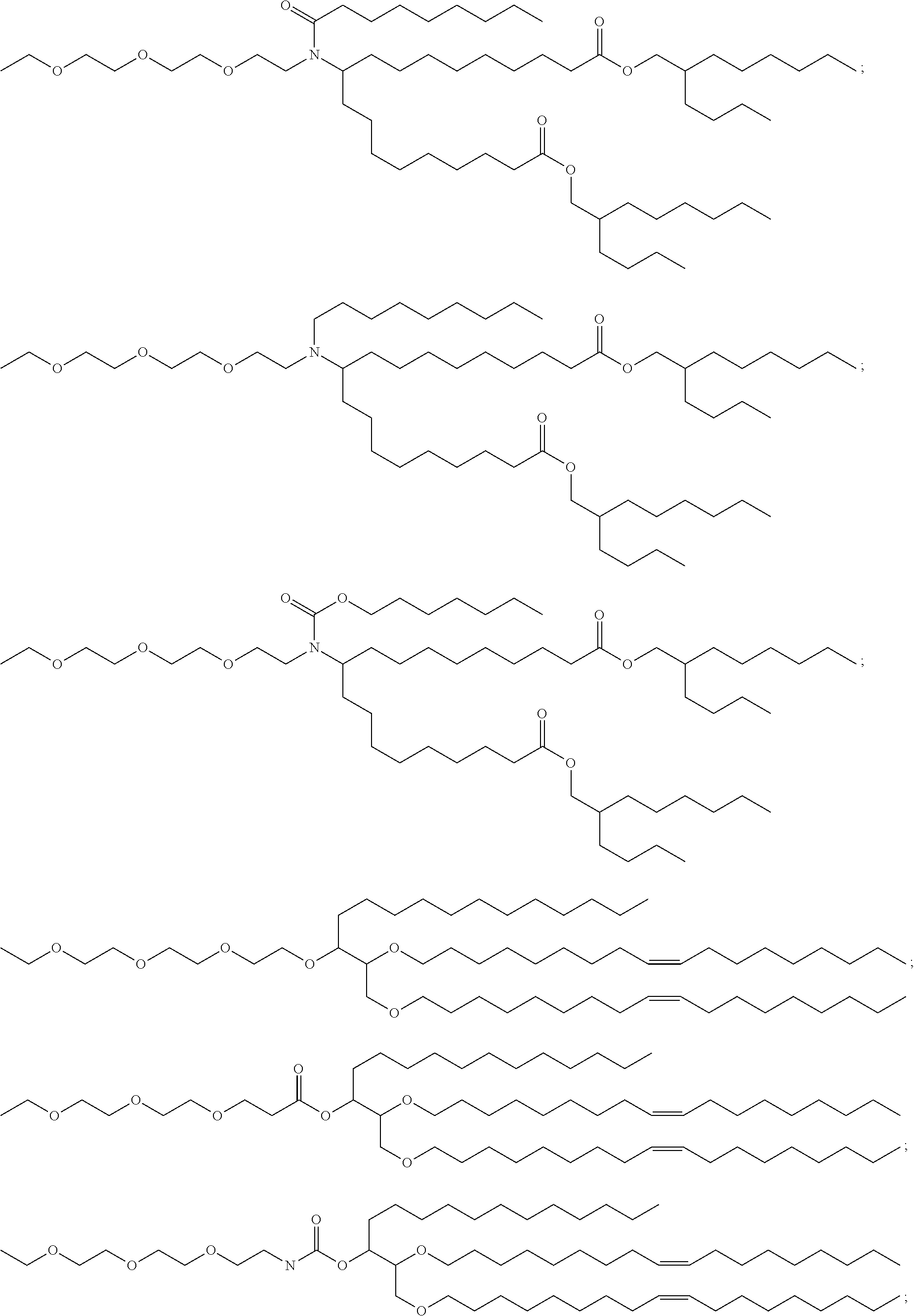

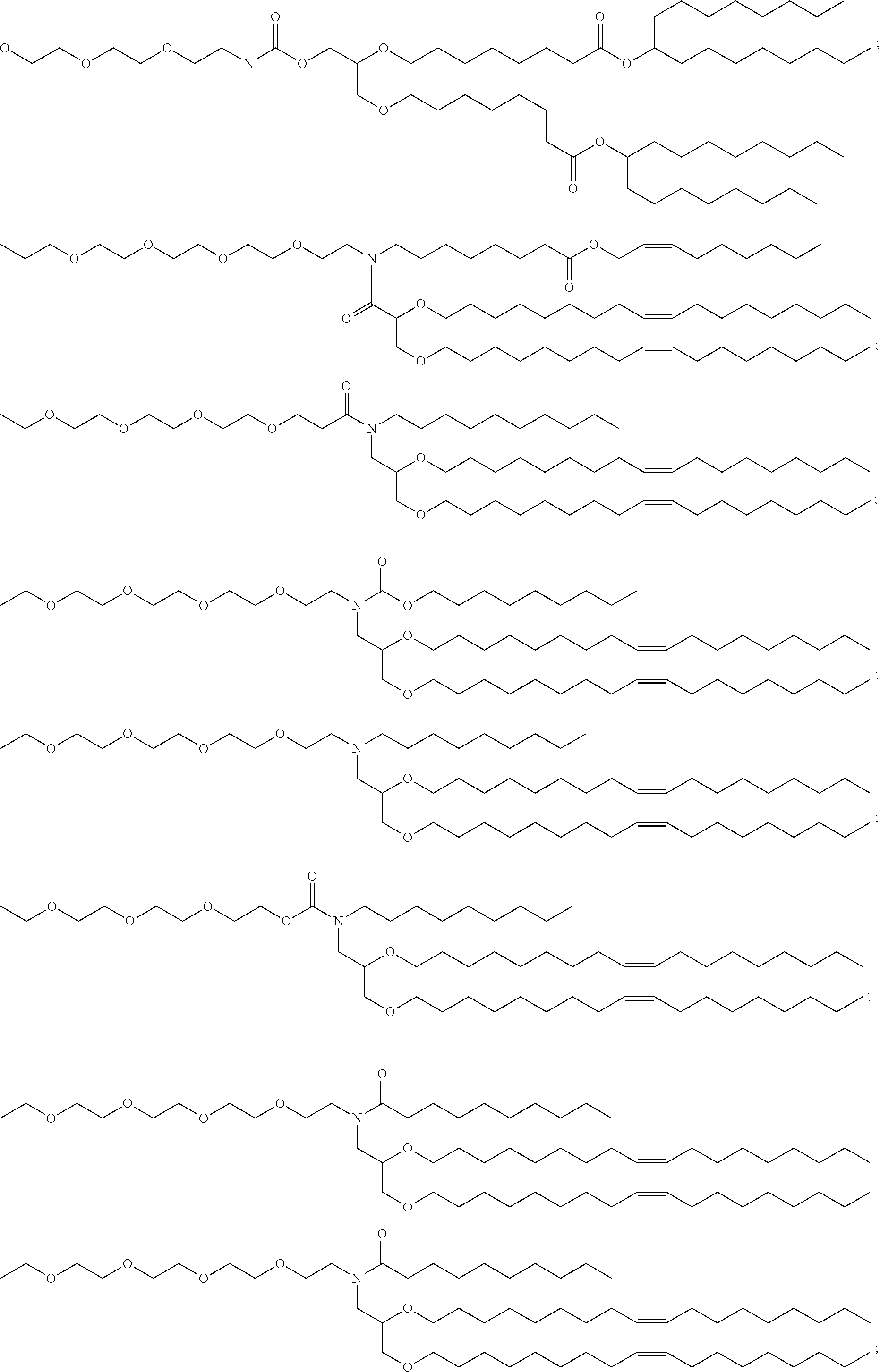

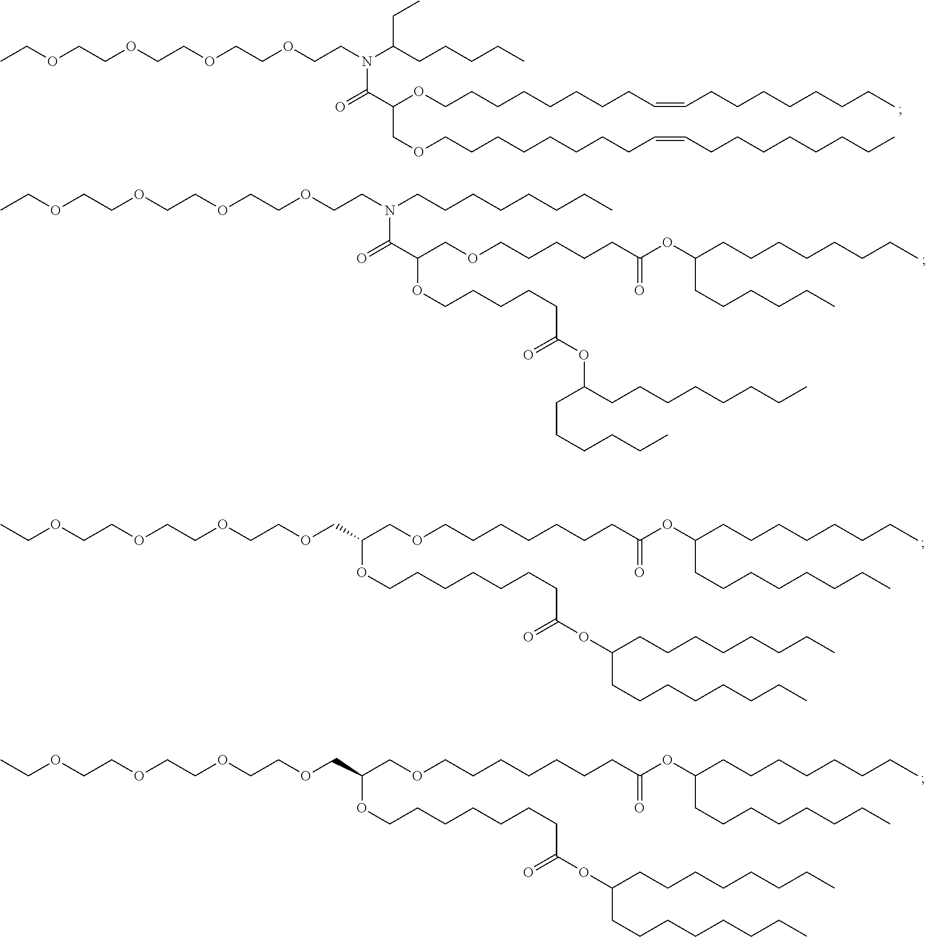

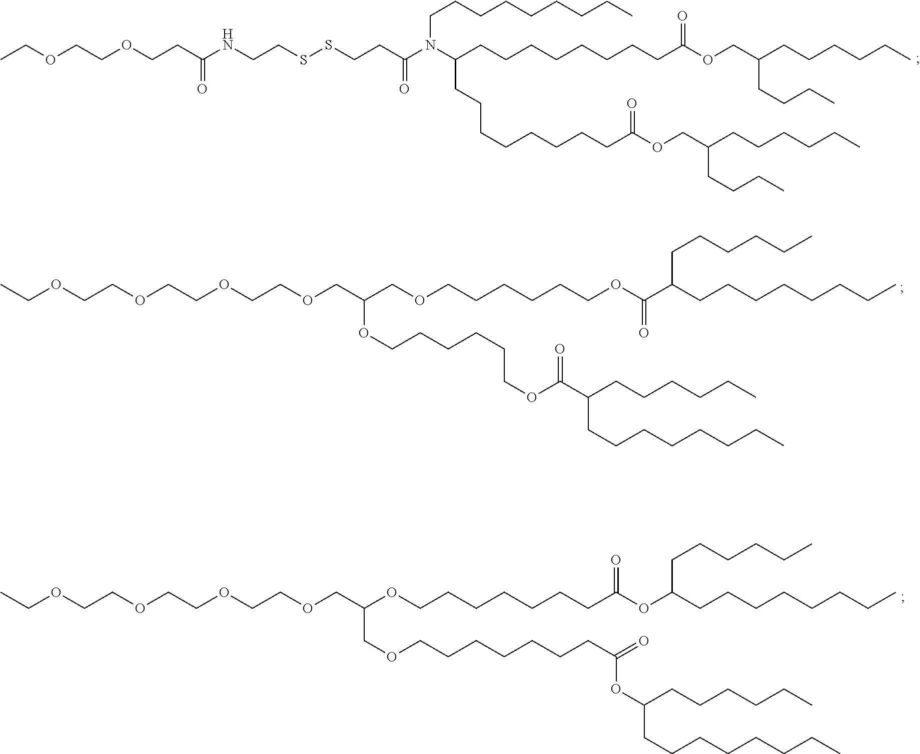

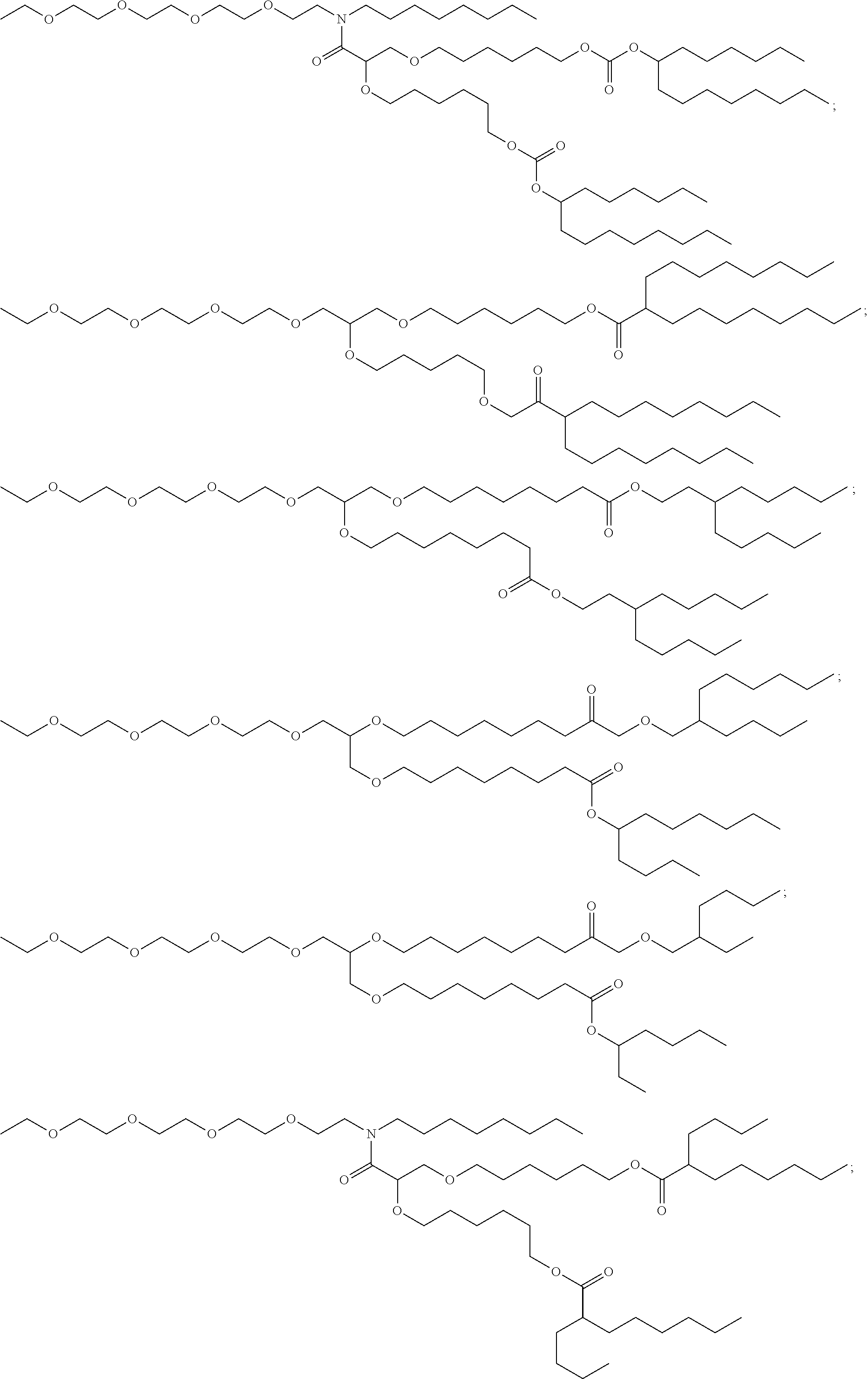

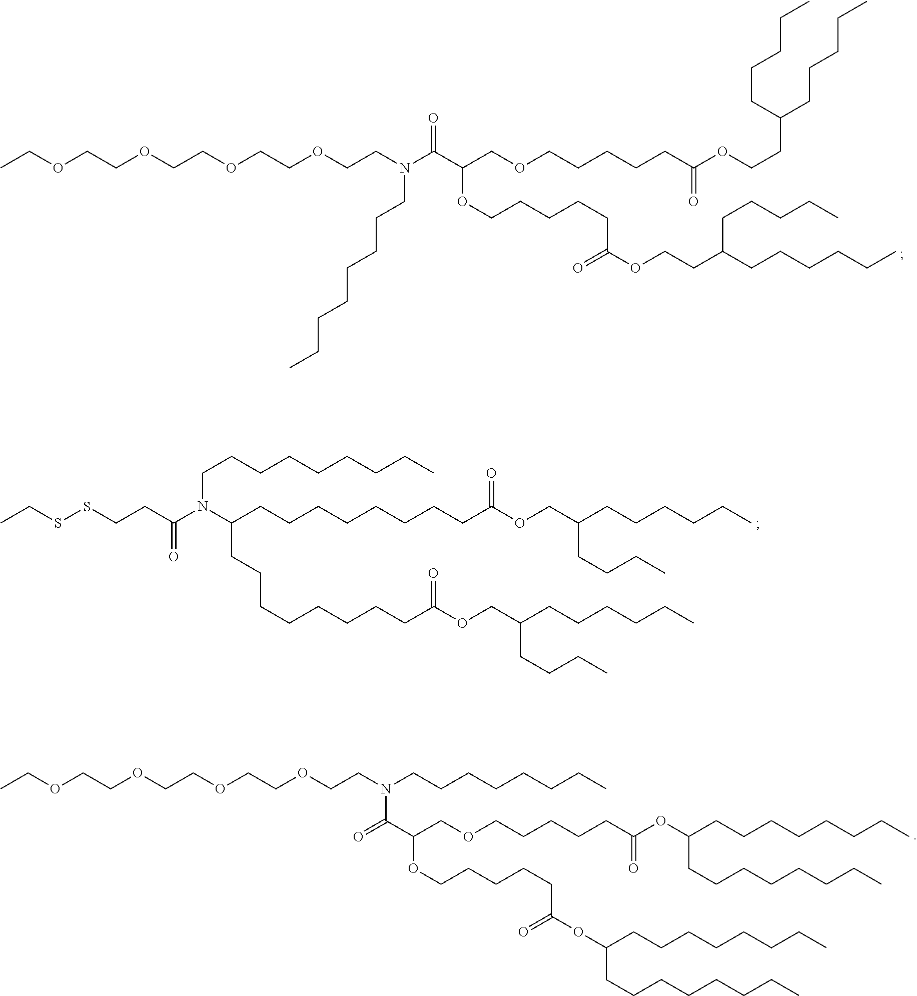

According to one embodiment, the radical A is a R2R3N-group in which R2 and R3 represent independently of each other a linear or branched (C₁-C₆) alkyl group, for example a dimethyl amino or a dipropylamino.

According to another embodiment, the radical A represents a NR2R3-Alk-Y-group in which Y is an oxygen or a nitrogen atom, Alk is a C₂ to C₆ alkylene and R2 and R3 represent independently of each other a linear or branched (C₁-C₆) alkyl group. For example, A is Aa or Ab

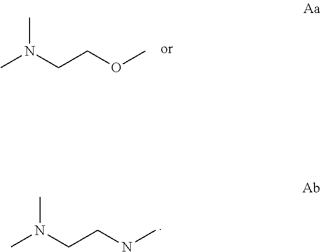

According to a specific embodiment, the radical A is selected in the group consisting of —N(CH₃)₂, —N(CH₂CH₂—CH₃)₂, —O—(CH₂)₂N(CH₃)₂ and —N—(CH₂)₂N(CH₃)₂.

According to another embodiment, the radical A represents a 4- to 8-membered saturated heterocyclic radical comprising 3 to 7 carbon atoms and 1 or 2 nitrogen atoms, said 4- to 8-membered saturated heterocyclic radical being linked to the rest of the molecule by a carbon atom or a nitrogen atom and being optionally substituted by 1 to 4 substituents, independently of each other, selected from a linear or branched (C₁-C₆) alkyl group. For instance, A is selected in the group consisting of piperidinyl radicals, piperazinyl radicals and pyrrolidinyl radicals. For example, A is selected in the group consisting of the 3 piperidinyl radical, 4 piperidinyl radical, 3 piperidinyl radical substituted by one or two methyl groups, 4 piperidinyl radical substituted by one or two methyl groups, 1-piperazinyl radical, 1-piperazinyl radical substituted by a methyl group, 3 pyrrolidinyl radical, and 3 pyrrolidinyl radical substituted by one or two methyl groups.

According to this specific embodiment, A is for example selected among piperidinyl, 1-methylpiperidinyl, 1,3-dimethylpiperidinyl, pyrrolidinyl and 1,3-dimethylpyrrolidinyl.

According to one embodiment, the compound of formula (I) has an apparent pKa lower than 7 or ranging from 4.5 to 7.

Another object of the disclosure relates to one compound of formula (II):

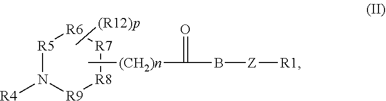

wherein
   Z, n and R1 are as defined here-above,
   R4 is a (C₁-C₅) alkyl group, for instance a (C₁-C₄) alkyl group, such as a methyl group;
   R12 is a (C₁-C₅) alkyl group, for instance a (C₁-C₄) alkyl group, such as methyl group;
   p is equal to zero or 1, and for instance p is equal to zero;
   R5, R6, R7, R8 and R9 are independently one of each other a moiety selected among —CH₂—; —CHR12— and —NH—, and the one of R5, R6, R7, R8 and R9 involved in the linkage with the rest of the molecule, being a moiety selected among —CH—; —CR12— and —N— and with the proviso that only one of R5, R6, R7, R8 and R9 is —NH— or —N—; and
   B represents an oxygen atom or a —NH— group, for instance an oxygen atom,
or one of its pharmaceutically acceptable salts thereof; and with said compound that is in all the possible racemic, enantiomeric and diastereoisomeric isomer forms.

Another object of the disclosure relates to one compound of formula (IIa):

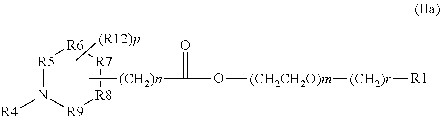

wherein
   R1 is as defined here-above;
   n is 0, 1, 2, 3, 4, 5 or 6, for instance 0 to 4, such as 0, 1 or 2;
   r is 0, 1, 2, 3 or 4, for instance 0, 1 or 2;
   R4 to R9, R12 and p are as defined here-above and for instance p is equal to zero,
   m is an integer from 1 to 12, for instance from 2 to 6, for example 4;
or one of its pharmaceutically acceptable salts thereof; and with said compound that is in all the possible racemic, enantiomeric and diastereoisomeric isomer forms.

Another object of the disclosure relates to one compound of formula (III):

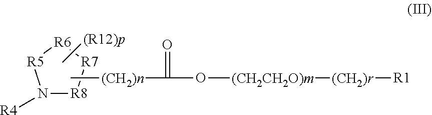

wherein
   R1 is as defined here-above;
   n is 0, 1, 2, 3, 4, 5 or 6, for instance 0 to 4, such as 0, 1 or 2;
   m is an integer from 1 to 12, for instance from 2 to 6, for example 4;
   r is 0, 1, 2, 3 or 4, for instance 0, 1 or 2 and;
   R4 to R8, R12 and p are as defined here-above and for instance p is equal to zero
or one of its pharmaceutically acceptable salts thereof; and with said compound that is in all the possible racemic, enantiomeric and diastereoisomeric isomer forms.

Another object of the disclosure relates to one compound of formula (IV):

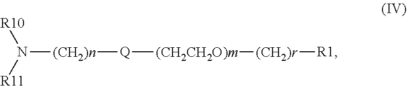

wherein
   R1 is as defined here-above;
   Q is a moiety selected in the group consisting of —O(CO)—*; —(C═O)—O*; —O(C═O)O*; —N(CO)O—* and —O(CO)N—* with * indicating the linking to the moiety (CH₂CH₂O) m
   n is 0, 1, 2, 3, 4, 5 or 6, for instance 1 to 5, for example 2, 3 or 4;
   r is 0, 1, 2, 3 or 4, for instance 0, 1 or 2;
   R10 and R11 represent independently of each other a (C₁-C₅) alkyl group, for instance a (C₁-C₄) alkyl group, such as a methyl group or a propyl group; and
   m is an integer from 1 to 12, for instance from 2 to 6, and for example 4;
or one of its pharmaceutically acceptable salts thereof; and with said compound that is in all the possible racemic, enantiomeric and diastereoisomeric isomer forms.

In some embodiments, Q may be a moiety selected in the group consisting of —(C═O)—O *; —O (C═O)O*; —N(C═O)O—* and —O(C═O)N—* with * indicating the linking to the moiety (CH₂CH₂O) m.

Another object of the disclosure relates to one compound of formula (V):

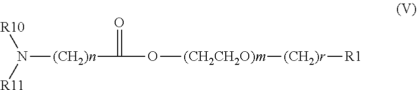

wherein
   R1, R10, R11, n, m and r are as defined here-above;
or one of its pharmaceutically acceptable salts thereof; and with said compound that is in all the possible racemic, enantiomeric and diastereoisomeric isomer forms.

According to one embodiment, the compounds of formula (II) are selected in the group consisting of the following compounds VI to XLVII of Table 1. In remark, in the following developed formula, a secondary amino moiety may be indifferently written —NH— or —N—:

TABLE 1
Compound VI (Example 1)

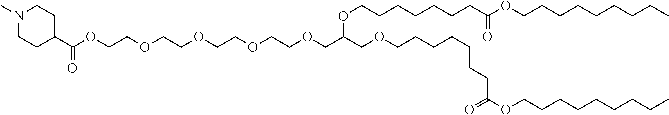

Compound VII (Example 2)

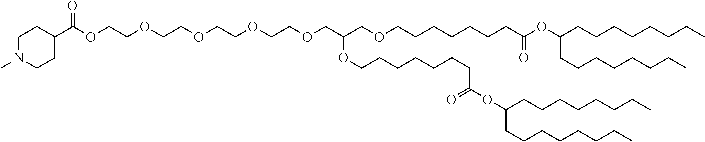 [SMILES: CCCCCCCCC(CCCCCCCC)OC(=O)CCCCCCCOCC(COCCOCCOCCOCCOC(=O)C1CCN(C)CC1)OCCCCCCCC(=O)OC(CCCCCCCC)CCCCCCCC]

Compound VIII (Example 3)

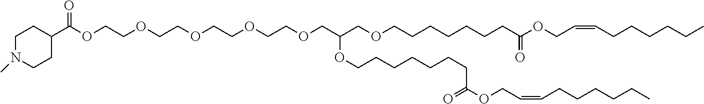 [SMILES: CCCCCC/C=C\COC(=O)CCCCCCCOCC(COCCOCCOCCOCCOC(=O)C1CCN(C)CC1)OCCCCCCCC(=O)OC/C=C\CCCCCC]

Compound IX (Example 4)

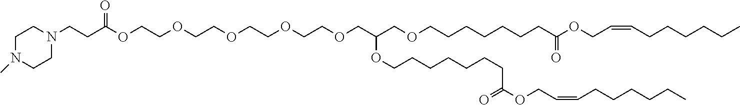 [SMILES: CCCCCC/C=C\COC(=O)CCCCCCCOCC(COCCOCCOCCOCCOC(=O)CCN1CCN(C)CC1)OCCCCCCCC(=O)OC/C=C\CCCCCC]

Compound X (Example 5)

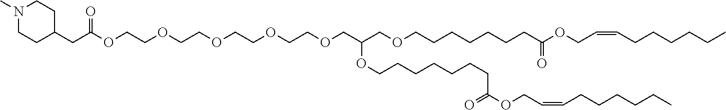 [SMILES: CCCCCC/C=C\COC(=O)CCCCCCCOCC(COCCOCCOCCOCCOC(=O)CC1CCN(C)CC1)OCCCCCCCC(=O)OC/C=C\CCCCCC]

Compound XI (Example 6)

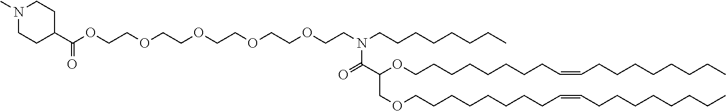 [SMILES: CCCCCCCC/C=C\CCCCCCCCOCC(OCCCCCCCC/C=C\CCCCCCCC)C(=O)N(CCCCCCCC)CCOCCOCCOCCOCCOC(=O)C1CCN(C)CC1]

Compound XII (Example 7)

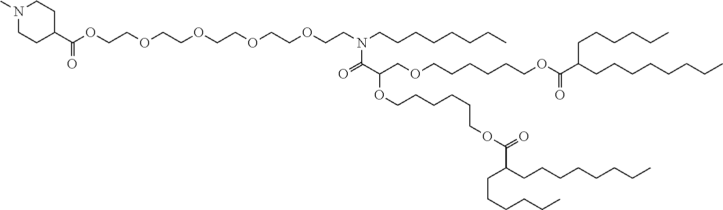 [SMILES: CCCCCCCCC(CCCCCC)C(=O)OCCCCCCOCC(OCCCCCCOC(=O)C(CCCCCC)CCCCCCCC)C(=O)N(CCCCCCCC)CCOCCOCCOCCOCCOC(=O)C1CCN(C)CC1]

Compound XIII (Example 8)

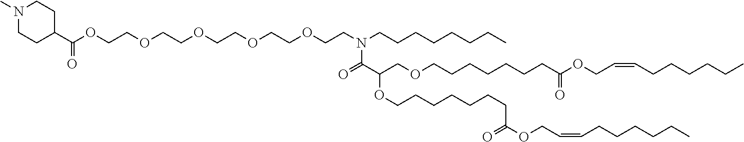 [SMILES: CCCCCC/C=C\COC(=O)CCCCCCCOCC(OCCCCCCCC(=O)OC/C=C\CCCCCC)C(=O)N(CCCCCCCC)CCOCCOCCOCCOCCOC(=O)C1CCN(C)CC1]

Compound XIV (Example 9)

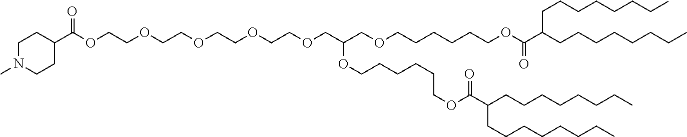 [SMILES: CCCCCCCCC(CCCCCCCC)C(=O)OCCCCCCOCC(COCCOCCOCCOCCOC(=O)C1CCN(C)CC1)OCCCCCCOC(=O)C(CCCCCCCC)CCCCCCCC]

Compound XV (Example 10)

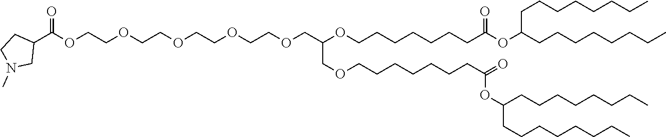 [SMILES: CCCCCCCCC(CCCCCCCC)OC(=O)CCCCCCCOCC(COCCOCCOCCOCCOC(=O)C1CCN(C)C1)OCCCCCCCC(=O)OC(CCCCCCCC)CCCCCCCC]

Compound XVI (Example 11)

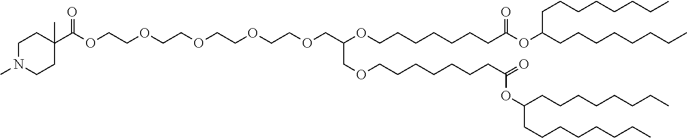 [SMILES: CCCCCCCCC(CCCCCCCC)OC(=O)CCCCCCCOCC(COCCOCCOCCOCCOC(=O)C1(C)CCN(C)CC1)OCCCCCCCC(=O)OC(CCCCCCCC)CCCCCCCC]

Compound XVII (Example 12)

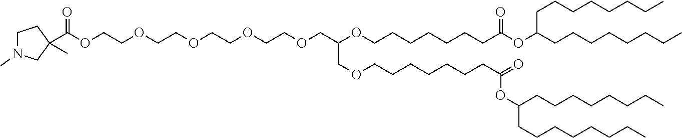 [SMILES: CCCCCCCCC(CCCCCCCC)OC(=O)CCCCCCCOCC(COCCOCCOCCOCCOC(=O)C1(C)CCN(C)C1)OCCCCCCCC(=O)OC(CCCCCCCC)CCCCCCCC]

Compound XVIII (Example 13)

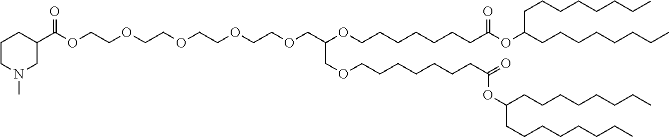 [SMILES: CCCCCCCCC(CCCCCCCC)OC(=O)CCCCCCCOCC(COCCOCCOCCOCCOC(=O)C1CCCN(C)C1)OCCCCCCCC(=O)OC(CCCCCCCC)CCCCCCCC]

Compound XIX (Example 14)

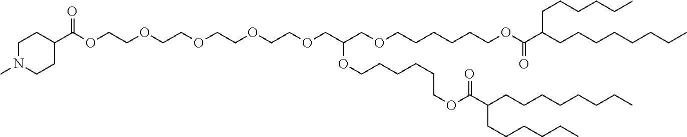 [SMILES: CCCCCCCCC(CCCCCC)C(=O)OCCCCCCOCC(COCCOCCOCCOCCOC(=O)C1CCN(C)CC1)OCCCCCCOC(=O)C(CCCCCC)CCCCCCCC]

Compound XX (Example 15)

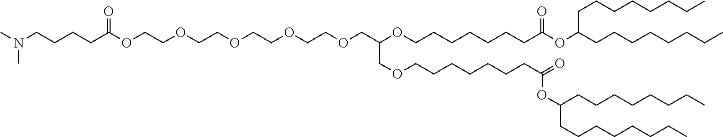 [SMILES: CCCCCCCCC(CCCCCCCC)OC(=O)CCCCCCCOCC(COCCOCCOCCOCCOC(=O)CCCCN(C)C)OCCCCCCCC(=O)OC(CCCCCCCC)CCCCCCCC]

Compound XXI (Example 16)

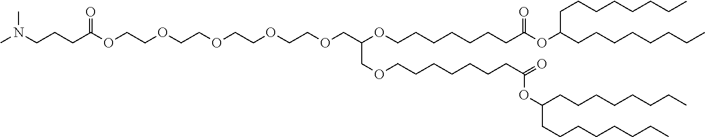 [SMILES: CCCCCCCCC(CCCCCCCC)OC(=O)CCCCCCCOCC(COCCOCCOCCOCCOC(=O)CCCN(C)C)OCCCCCCCC(=O)OC(CCCCCCCC)CCCCCCCC]

Compound XXII (Example 17)

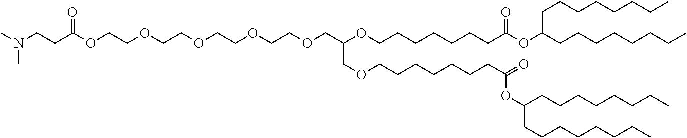 [SMILES: CCCCCCCCC(CCCCCCCC)OC(=O)CCCCCCCOCC(COCCOCCOCCOCCOC(=O)CCN(C)C)OCCCCCCCC(=O)OC(CCCCCCCC)CCCCCCCC]

Compound XXIII (Example 18)

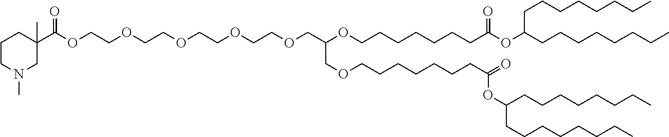 [SMILES: CCCCCCCCC(CCCCCCCC)OC(=O)CCCCCCCOCC(COCCOCCOCCOCCOC(=O)C1(C)CCCN(C)C1)OCCCCCCCC(=O)OC(CCCCCCCC)CCCCCCCC]

Compound XXVII (Example 19)

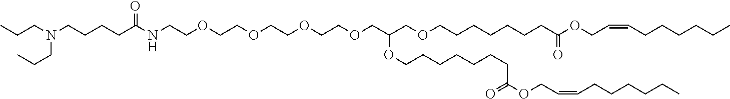 [SMILES: CCCCCC/C=C\COC(=O)CCCCCCCOCC(COCCOCCOCCOCCNC(=O)CCCCN(CCC)CCC)OCCCCCCCC(=O)OC/C=C\CCCCCC]

Compound XXVIII (Example 20)

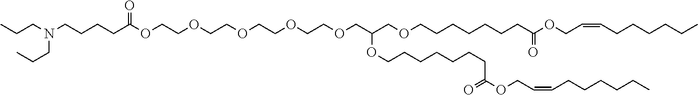 [SMILES: CCCCCC/C=C\COC(=O)CCCCCCCOCC(COCCOCCOCCOCCOC(=O)CCCCN(CCC)CCC)OCCCCCCCC(=O)OC/C=C\CCCCCC]

Compound XXIX (Example 21)

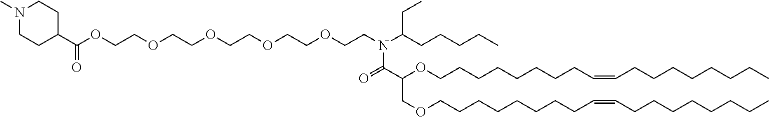 [SMILES: CCCCCCCC/C=C\CCCCCCCCOCC(OCCCCCCCC/C=C\CCCCCCCC)C(=O)N(CCOCCOCCOCCOCCOC(=O)C1CCN(C)CC1)C(CC)CCCCC]

Compound XXX (Example 22)

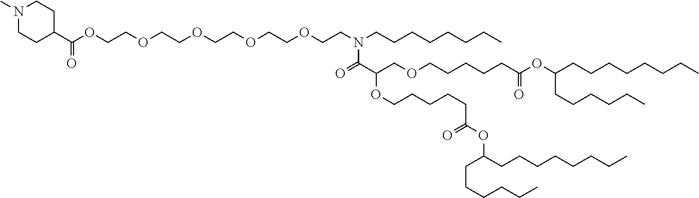 [SMILES: CCCCCCCCC(CCCCCC)OC(=O)CCCCCOCC(OCCCCCC(=O)OC(CCCCCC)CCCCCCCC)C(=O)N(CCCCCCCC)CCOCCOCCOCCOCCOC(=O)C1CCN(C)CC1]

Compound XXXI (Example 23)

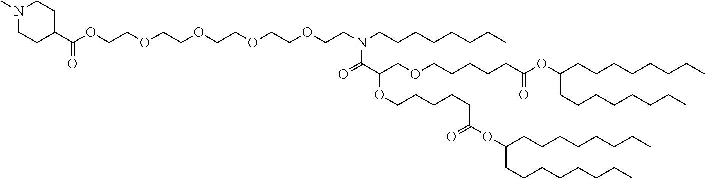 [SMILES: CCCCCCCCC(CCCCCCCC)OC(=O)CCCCCOCC(OCCCCCC(=O)OC(CCCCCCCC)CCCCCCCC)C(=O)N(CCCCCCCC)CCOCCOCCOCCOCCOC(=O)C1CCN(C)CC1]

Compound XXXII (Example 24) (chiral)

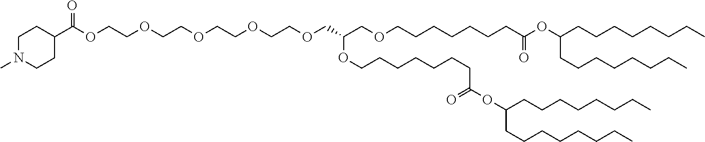 [SMILES: CCCCCCCCC(CCCCCCCC)OC(=O)CCCCCCCOC[C@@H](COCCOCCOCCOCCOC(=O)C1CCN(C)CC1)OCCCCCCCC(=O)OC(CCCCCCCC)CCCCCCCC]

Compound XXXIII (Example 25) (chiral)

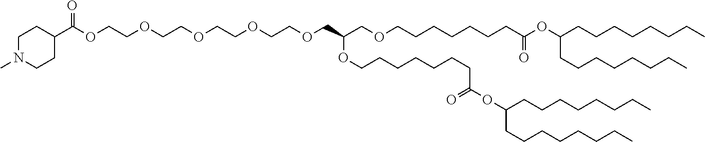 [SMILES: CCCCCCCCC(CCCCCCCC)OC(=O)CCCCCCCOC[C@H](COCCOCCOCCOCCOC(=O)C1CCN(C)CC1)OCCCCCCCC(=O)OC(CCCCCCCC)CCCCCCCC]

Compound XXXIV (Example 26)

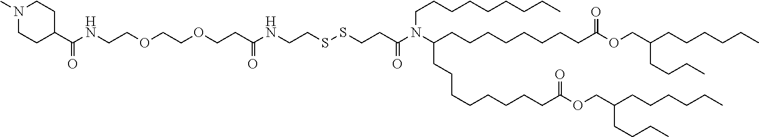 [SMILES: CCCCCCCCCN(C(=O)CCSSCCNC(=O)CCOCCOCCNC(=O)C1CCN(C)CC1)C(CCCCCCCCC(=O)OCC(CCCC)CCCCCC)CCCCCCCCC(=O)OCC(CCCC)CCCCCC]

Compound XXXV (Example 27)

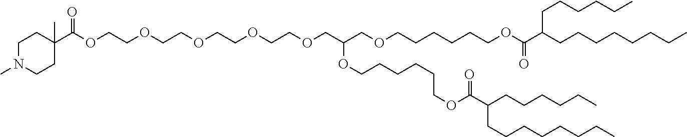 [SMILES: CCCCCCCCC(CCCCCC)C(=O)OCCCCCCOCC(COCCOCCOCCOCCOC(=O)C1(C)CCN(C)CC1)OCCCCCCOC(=O)C(CCCCCC)CCCCCCCC]

Compound XXXVI (Example 28)

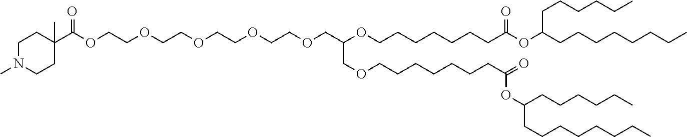 [SMILES: CCCCCCCCC(CCCCCC)OC(=O)CCCCCCCOCC(COCCOCCOCCOCCOC(=O)C1(C)CCN(C)CC1)OCCCCCCCC(=O)OC(CCCCCC)CCCCCCCC]

Compound XXXVII (Example 29)

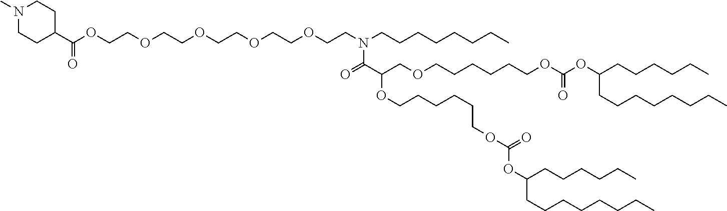 [SMILES: CCCCCCCCC(CCCCCC)OC(=O)OCCCCCCOCC(OCCCCCCOC(=O)OC(CCCCCC)CCCCCCCC)C(=O)N(CCCCCCCC)CCOCCOCCOCCOCCOC(=O)C1CCN(C)CC1]

Compound XXXVIII (Example 30)

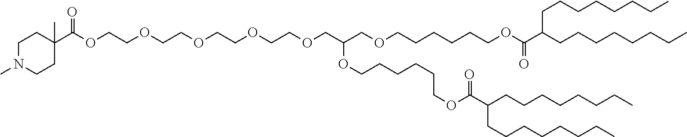 [SMILES: CCCCCCCCC(CCCCCCCC)C(=O)OCCCCCCOCC(COCCOCCOCCOCCOC(=O)C1(C)CCN(C)CC1)OCCCCCCOC(=O)C(CCCCCCCC)CCCCCCCC]

Compound XXXIX (Example 31)

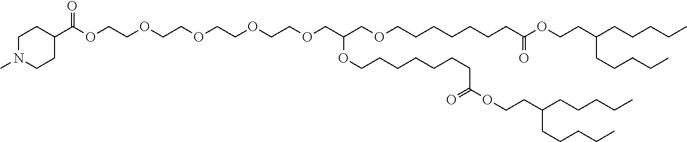 [SMILES: CCCCCC(CCCCC)CCOC(=O)CCCCCCCOCC(COCCOCCOCCOCCOC(=O)C1CCN(C)CC1)OCCCCCCCC(=O)OCCC(CCCCC)CCCCC]

Compound XLVII(Example 32)

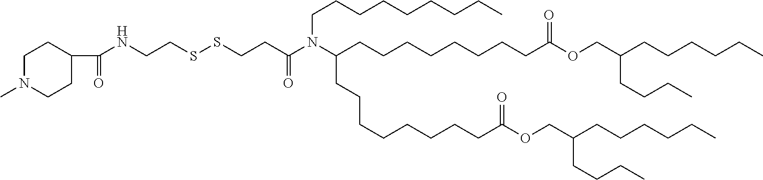 [SMILES: CCCCCCCCCN(C(=O)CCSSCCNC(=O)C1CCN(C)CC1)C(CCCCCCCCC(=O)OCC(CCCC)CCCCCC)CCCCCCCCC(=O)OCC(CCCC)CCCCCC]

Compound XLI (Example 33)

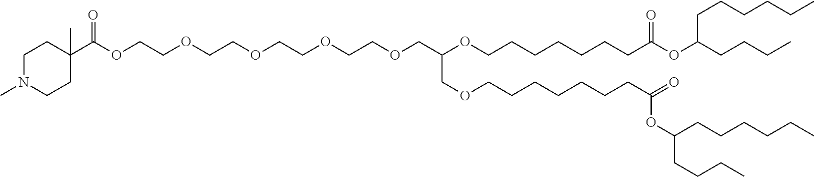 [SMILES: CCCCCCC(CCCC)OC(=O)CCCCCCCOCC(COCCOCCOCCOCCOC(=O)C1(C)CCN(C)CC1)OCCCCCCCC(=O)OC(CCCC)CCCCCC]

Compound XLII (Example 34)

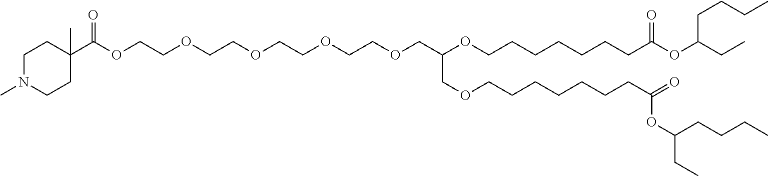 [SMILES: CCCCC(CC)OC(=O)CCCCCCCOCC(COCCOCCOCCOCCOC(=O)C1(C)CCN(C)CC1)OCCCCCCCC(=O)OC(CC)CCCC]

Compound XLIII (Example 35)

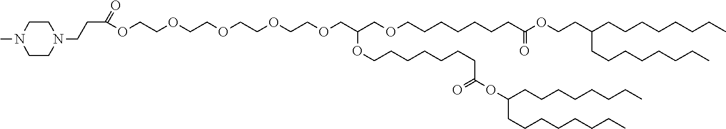

Compound XLIV (Example 36)

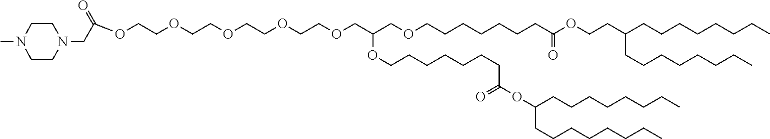

Compound XLV (Example 37)

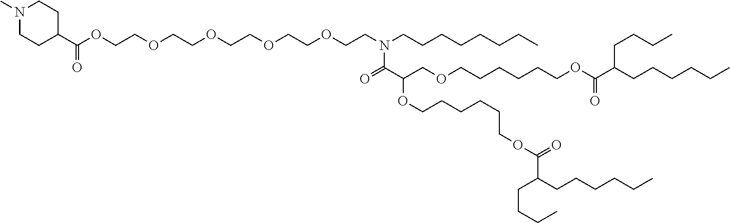 [SMILES: CCCCCCCCN(CCOCCOCCOCCOCCOC(=O)C1CCN(C)CC1)C(=O)C(COCCCCCCOC(=O)C(CCCC)CCCCCC)OCCCCCCOC(=O)C(CCCC)CCCCCC]

Compound XLVI (Example 38)

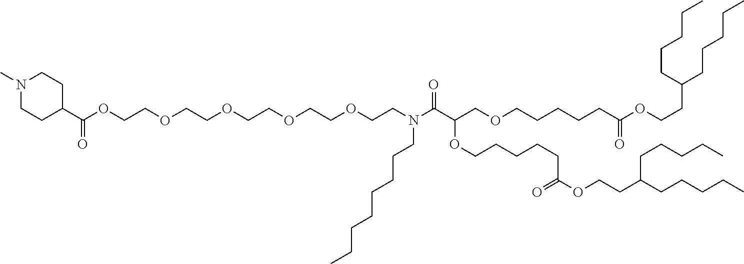 [SMILES: CCCCCCCCN(CCOCCOCCOCCOCCOC(=O)C1CCN(C)CC1)C(=O)C(COCCCCCC(=O)OCCC(CCCCC)CCCCC)OCCCCCC(=O)OCCC(CCCCC)CCCCC]

Compound XXIV (Example 39)

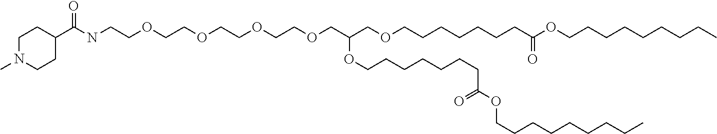 [SMILES: CCCCCCCCCOC(=O)CCCCCCCOCC(COCCOCCOCCOCCNC(=O)C1CCN(C)CC1)OCCCCCCCC(=O)OCCCCCCCCC]

Compound XXV (Example 40)

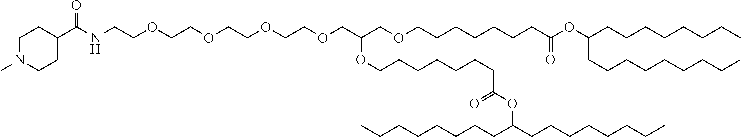 [SMILES: CCCCCCCCCC(CCCCCCCC)OC(=O)CCCCCCCOCC(COCCOCCOCCOCCNC(=O)C1CCN(C)CC1)OCCCCCCCC(=O)OC(CCCCCCCC)CCCCCCCC]

Compound XXVI (Example 41)

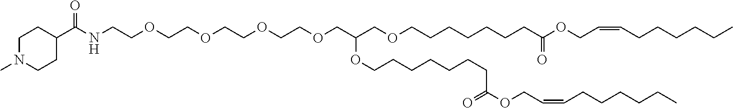 [SMILES: CCCCCC/C=C\COC(=O)CCCCCCCOCC(COCCOCCOCCOCCNC(=O)C1CCN(C)CC1)OCCCCCCCC(=O)OC/C=C\CCCCCC]

or one of their pharmaceutically acceptable salts thereof; and with said compounds that are in all the possible racemic, enantiomeric and diastereoisomeric isomer forms.

According to one specific embodiment, the compounds of formula (I) are selected among the compounds listed in table 2.

TABLE 2
Compound VII (Example 2)

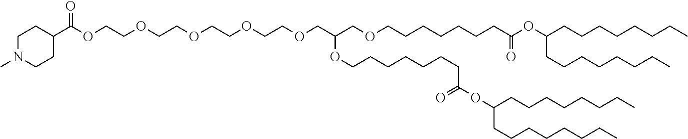 [SMILES: CCCCCCCCC(CCCCCCCC)OC(=O)CCCCCCCOCC(COCCOCCOCCOCCOC(=O)C1CCN(C)CC1)OCCCCCCCC(=O)OC(CCCCCCCC)CCCCCCCC]

Compound XII (Example 7)

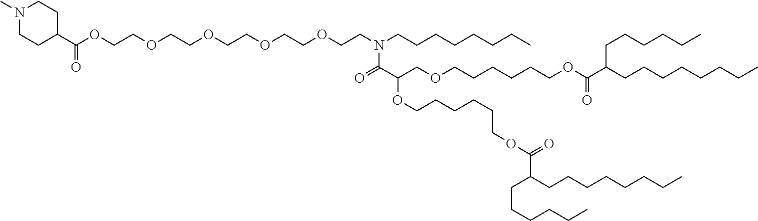 [SMILES: CCCCCCCCC(CCCCCC)C(=O)OCCCCCCOCC(OCCCCCCOC(=O)C(CCCCCC)CCCCCCCC)C(=O)N(CCCCCCCC)CCOCCOCCOCCOCCOC(=O)C1CCN(C)CC1]

Compound XIV (Example 9)

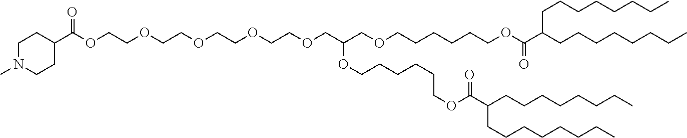 [SMILES: CCCCCCCCC(CCCCCCCC)C(=O)OCCCCCCOCC(COCCOCCOCCOCCOC(=O)C1CCN(C)CC1)OCCCCCCOC(=O)C(CCCCCCCC)CCCCCCCC]

Compound XVI (Example 11)

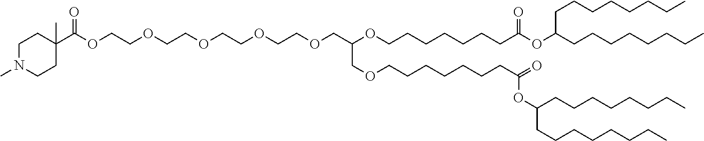 [SMILES: CCCCCCCCC(CCCCCCCC)OC(=O)CCCCCCCOCC(COCCOCCOCCOCCOC(=O)C1(C)CCN(C)CC1)OCCCCCCCC(=O)OC(CCCCCCCC)CCCCCCCC]

Compound XVIII (Example 13)

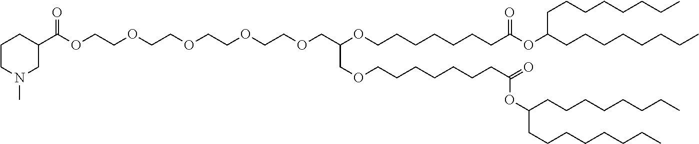 [SMILES: CCCCCCCCC(CCCCCCCC)OC(=O)CCCCCCCOCC(COCCOCCOCCOCCOC(=O)C1CCCN(C)C1)OCCCCCCCC(=O)OC(CCCCCCCC)CCCCCCCC]

Compound XIX (Example 14)

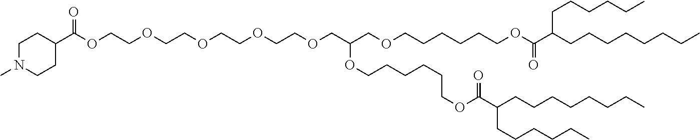 [SMILES: CCCCCCCCC(CCCCCC)C(=O)OCCCCCCOCC(COCCOCCOCCOCCOC(=O)C1CCN(C)CC1)OCCCCCCOC(=O)C(CCCCCC)CCCCCCCC]

Compound XXX (Example 22)

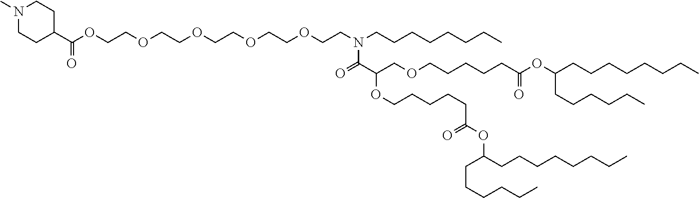 [SMILES: CCCCCCCCC(CCCCCC)OC(=O)CCCCCOCC(OCCCCCC(=O)OC(CCCCCC)CCCCCCCC)C(=O)N(CCCCCCCC)CCOCCOCCOCCOCCOC(=O)C1CCN(C)CC1]

Compound XXXI (Example 23)

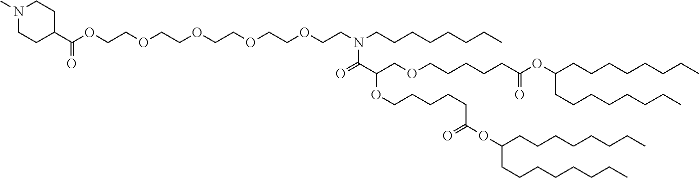 [SMILES: CCCCCCCCC(CCCCCCCC)OC(=O)CCCCCOCC(OCCCCCC(=O)OC(CCCCCCCC)CCCCCCCC)C(=O)N(CCCCCCCC)CCOCCOCCOCCOCCOC(=O)C1CCN(C)CC1]

Compound XXXII (Example 24)

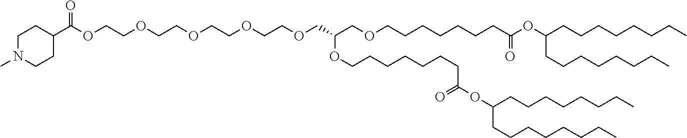 [SMILES: CCCCCCCCC(CCCCCCCC)OC(=O)CCCCCCCOC[C@@H](COCCOCCOCCOCCOC(=O)C1CCN(C)CC1)OCCCCCCCC(=O)OC(CCCCCCCC)CCCCCCCC]

Compound XXXIII (Example 25)

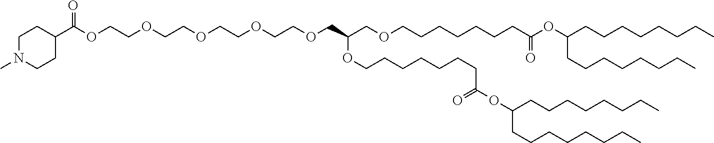 [SMILES: CCCCCCCCC(CCCCCCCC)OC(=O)CCCCCCCOC[C@H](COCCOCCOCCOCCOC(=O)C1CCN(C)CC1)OCCCCCCCC(=O)OC(CCCCCCCC)CCCCCCCC]

Compound XXXIV (Example 26)

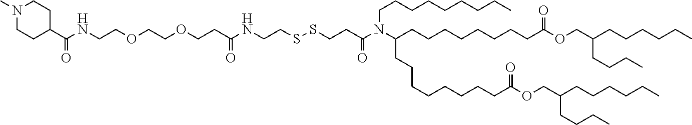 [SMILES: CCCCCCCCCN(C(=O)CCSSCCNC(=O)CCOCCOCCNC(=O)C1CCN(C)CC1)C(CCCCCCCCC(=O)OCC(CCCC)CCCCCC)CCCCCCCCC(=O)OCC(CCCC)CCCCCC]

Compound XXXV (Example 27)

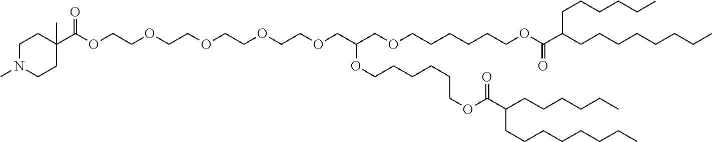 [SMILES: CCCCCCCCC(CCCCCC)C(=O)OCCCCCCOCC(COCCOCCOCCOCCOC(=O)C1(C)CCN(C)CC1)OCCCCCCOC(=O)C(CCCCCC)CCCCCCCC]

Compound XXXVI (Example 28)

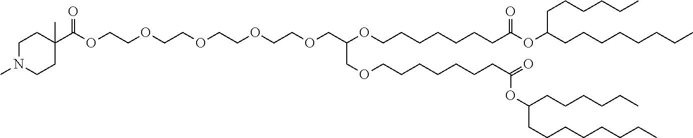 [SMILES: CCCCCCCCC(CCCCCC)OC(=O)CCCCCCCOCC(COCCOCCOCCOCCOC(=O)C1(C)CCN(C)CC1)OCCCCCCCC(=O)OC(CCCCCC)CCCCCCCC]

Compound XXXVII (Example 29)

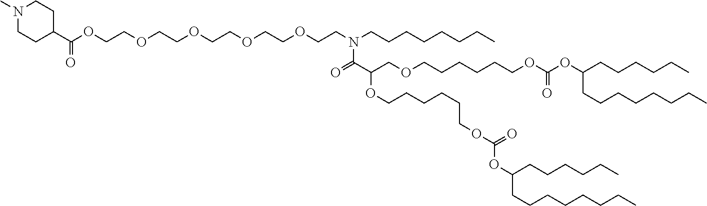 [SMILES: CCCCCCCCC(CCCCCC)OC(=O)OCCCCCCOCC(OCCCCCCOC(=O)OC(CCCCCC)CCCCCCCC)C(=O)N(CCCCCCCC)CCOCCOCCOCCOCCOC(=O)C1CCN(C)CC1]

Compound XXXVIII (Example 30)

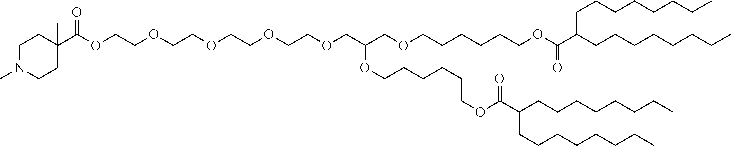 [SMILES: CCCCCCCCC(CCCCCCCC)C(=O)OCCCCCCOCC(COCCOCCOCCOCCOC(=O)C1(C)CCN(C)CC1)OCCCCCCOC(=O)C(CCCCCCCC)CCCCCCCC]

or one of their pharmaceutically acceptable salts thereof; and with said compounds that are in all the possible racemic, enantiomeric and diastereoisomeric isomer forms.

The compounds according to the disclosure can be prepared from readily commercially available or described in the literature starting materials using methods and procedures known from the skilled person.

### Compositions, Lipid Nanoparticles, and Manufacturing Processes

Compositions or lipid nanoparticles (LNPs) may comprise a lipid component comprising at least a lipidic compound as disclosed herein.

The lipid component may further comprise at least a lipid selected from a neutral lipid, a structural lipid, and optionally a PEG-lipid.

Composition or LNPs may comprise at least one biologically active agent. A biologically active agent may be a prophylactic agent, a therapeutic agent, immunomodulatory agent, or a diagnostic agent.

### Ionizable Cationic Lipids

The lipid component of the compositions or the LNPs comprises at least one lipidic compound as described herein. Such lipidic compound is an ionizable cationic lipid.

The compositions or the LNPs may comprise from about 20 to about 60%, or from about 25% to about 60%, or from about 30% to about 55%, or from about 40% to about 55%, or from about 40% to about 50%, of molar amount of lipidic compound disclosed herein, in % relative to the total molar amount of the lipid components.

The compositions or the LNPs may comprise from about 20%, about 25, about 30, about 35, about 40, about 45, about 50, about 55, or about 60%, of molar amount of lipidic compound disclosed herein, in % relative to the total molar amount of the lipid components.

A lipidic compound disclosed herein may be present in an amount of about 50% relative to the total weight of the lipid components of the compositions or LNPs.

In one embodiment, a suitable lipidic compound may be a lipidic compound of formula (I).

In one embodiment, a suitable lipidic compound may be a lipidic compound of formula (II).

In one embodiment, a suitable lipidic compound may be a lipidic compound of formula (IIa).

In one embodiment, a suitable lipidic compound may be a lipidic compound of formula (III).

In one embodiment, a suitable lipidic compound may be a lipidic compound of formula (IV).

In one embodiment, a suitable lipidic compound may be a lipidic compound of formula (V).

In one embodiment, a suitable lipidic compound may be a lipidic compound as indicated in Table 1.

In one embodiment, a suitable lipidic compound may be a lipidic compound as indicated in Table 2.

In some embodiments, a lipidic compound may be of formula (VI), (VII), (VIII), (XI), (XII), (XIV), (XV), (XVI), (XVII), (XVIII), (XIX), (XX), (XXI), (XXX), (XXXI), (XXXII), (XXXIII), (XXXIV), (XXXV), (XXXVI), (XXXVII) or (XXXVIIII).

In some embodiments, a lipidic compound may be of formula (VI), (VII), (XII), (XIV), (XVI), (XVIII), (XIX), (XXI), (XXX), (XXXI), (XXXII), (XXXIII), (XXXIV), (XXXV), (XXXVI), (XXXVII) or (XXXVIIII).

In some embodiments, a lipidic compound may be of formula (VI), (VII), (VIII), (XI), or (XII).

In some embodiments, a lipidic compound may be of formula (VI), (VII), (VIII), or (XII).

In some embodiments, a lipidic compound may be of formula (VII), (XII), (XIV), (XV), (XVI), (XIX), (XX) or (XXI).

In some embodiments, a lipidic compound may be of formula (VII), (XII), (XIV), (XVI), (XIX), or (XXI).

In some embodiments, a lipidic compound may be of formula (VI), (VII) or (XII).

In some embodiments, a lipidic compound may be of formula (XIV), (XVI), (XVIII), (XIX), (XXX), (XXXI), (XXXII), (XXXIII), (XXXIV), (XXXV), (XXXVI), (XXXVII) or (XXXVIIII).

In some embodiments, a lipidic compound may be of formula (XXX), (XXXI), (XXXII), (XXXIII), (XXXIV), (XXXV), (XXXVI), (XXXVII) or (XXXVIIII).

In some embodiments, a lipidic compound may be of formula (XIV), (XVI), (XVIII), (XXX), (XXXII), (XXXIII), (XXXIV), (XXXV), (XXXVI), or (XXXVIIII).

In some embodiments, a lipidic compound may be of formula (XVI), (XVIII), (XXXIII), (XXXIV), or (XXXVIII).

In some embodiments, a lipidic compound may be of formula (XVIII), (XXXIII), (XXXIV), or (XXXVIII).

In some embodiments, a lipidic compound may be of formula (XVIII), (XXXIII), or (XXXVIII).

### Neutral Lipids

The lipid component of the compositions or the LNPs may include a neutral lipid. The presence of neutral lipids may improve structural stability of the lipid nanoparticles. The neutral lipid can be appropriately selected in view of the delivery efficiency of a biologically active agent, such as a nucleic acid.

The neutral lipids are distinct from the lipidic compounds disclosed herein. Neutral lipids are either not ionizable or are neutral zwitterionic compounds at a selected pH.

Neutral lipids may be selected from the group consisting of phosphatidylcholines, phosphatidylethanolamines, sphingomyelins, and ceramides.

Phosphatidylcholines and phosphatidylethanolamines are zwitterionic lipids. Sphingomyelins and ceramides are not ionizable lipids.

As examples of phosphatidylcholines, one may mention DSPC (1,2-distearoyl-sn-glycero-3-phosphocholine), DPPC (1,2-dipalmitoyl-sn-glycero-3-phosphocholine), DMPC (1,2-dimyristoyl-sn-glycero-3-phosphocholine), POPC (1-palmitoyl-2-oleoyl-sn-glycero-3-phosphocholine), DOPC (1,2-dioleoyl-sn-glycero-3-phosphocholine), and mixtures thereof.

As examples of phosphatidylethanolamines, one may mention DOPE (1,2-dioleyl-sn-glycero-3-phosphoethanolamine), DPPE (1,2-dipalmitoyl-sn-glycero-3-phosphoethanolamine), DMPE (1,2-dimyristoyl-sn-glycero-3-phosphoethanolamine), DSPE (1,2-distearoyl-s/i-glycero-3-phosphoethanolamine), DLPE (1,2-dilauroyl-SM-glycero-3-phosphoethanolamine), DEPE (1,2-dierucoyl-sn-glycero-3-phosphoethanolamine), 16-O-monomethyl PE, 16-O-dimethyl PE, 18-1-trans PE, or I-stearoyl-2-oleoyl-phosphatidyethanolamine (SOPE), and mixtures thereof.

A neutral lipid may be selected from the group consisting of phosphatidylcholines, such as DSPC, DPPC, DMPC, POPC, DOPC; phosphatidylethanolamines, such as DOPE, DPPE, DMPE, DSPE, DLPE, DEPE; sphingomyelins; ceramides; and mixtures thereof.

In one embodiment, a neutral lipid may be DEPE, DSPC, DOPC, or DOPE, or a mixture thereof, and for example may be DEPE, DSPC or DOPE, or a mixture thereof.

In one embodiment, a neutral lipid may be DSPC, DOPC, or DOPE, or a mixture thereof, and for example may be DSPC or DOPE, or a mixture thereof.

In one embodiment, a neutral lipid may be DSPC.

The compositions or the LNPs may comprise at least a neutral lipid in a molar amount ranging from about 0% to about 50%, from about 5% to about 45%, from about 8% to about 40%, or from about 10% to about 30%, in % relative to the total molar amount of the lipid component of the compositions or LNPs.

The compositions or the LNPs may comprise at least a neutral lipid in a molar amount of about 0%, about 5, about 10, about 15, about 20, about 25, about 30, about 35, about 40, about 45, or about 50%, in % relative to the total molar amount of the lipid component of the compositions or LNPs.

Neutral lipids may be present in compositions or LNPs in a molar ratio lipidic compound: neutral lipid which may range from about 70:1 to about 1:2, from about 30:1 to about 1:1, from about 15:1 to about 2:1, from about 10:1 to about 4:1, or at about 5:1.

### Structural Lipids

The lipid component of the compositions or the LNPs may include a structural lipid. The presence of a structural lipid, such as a sterol or an ester of sterol, may improve structural stability of the lipid nanoparticles

The LNPs as disclosed herein may comprise at least one a steroid alcohol (or sterol) or an ester thereof.

A structural lipid may be selected from the group consisting of a sterol or an ester thereof, tomatine, alpha-tocopherol, corticosteroid, and mixtures thereof.

A sterol may be selected from the group consisting of cholesterol or its derivatives, ergosterol, desmosterol (3β-hydroxy-5,24-cholestadiene), stigmasterol (stigmasta-5,22-dien-3-ol), lanosterol (8,24-lanostadien-3β-ol), 7-dehydrocholesterol (Δ5,7-cholesterol), dihydrolanosterol (24,25-dihydrolanosterol), zymosterol (5a-cholesta-8,24-dien-3β-ol), lathosterol (5a-cholest-7-en-3B-ol), diosgenin ((3β,25R)-spirost-5-en-3-ol), sitosterol (22,23-dihydrostigmasterol), sitostanol, campesterol (campest-5-en-3β-ol), campestanol (5a-campestan-3b-ol), fecosterol, brassicasterol, tomatidine, ursolic acid, 24-methylene cholesterol (5,24(28)-cholestadien-24-methylen-3β-ol); BHEM-Cholesterol (2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a] 79 henanthrene-3-yl)oxy) carbonyl) amino)-N,N-bis (2-hydroxyethyl)-N-methylethan-1-aminium bromide); and mixtures thereof.

Esters of sterol refer to ester of carboxylic acid with the hydroxyl group of the steroid alcohol. Suitable carboxylic acid comprises, further to the carboxyl moiety, a saturated or unsaturated, linear or branched, alkyl group. In some embodiments, the alkyl group may be a C₁-C₂₀ saturated or unsaturated, linear or branched, alkyl group, for example a C₂-C₁₈, for example a C₄-C₁₆, for example C₈-C₁₂ saturated or unsaturated, linear or branched, alkyl group. In other embodiments, the carboxylic acid may be a fatty acid. For example, a fatty acid may be caprylic acid, capric acid, lauric acid, stearic acid, margaric acid, oleic acid, linoleic acid, or arachidic acid.

Esters of sterol may be selected from the group consisting of cholesteryl margarate (cholest-5-en-3β-yl heptadecanoate), cholesteryl oleate, cholesteryl stearate; and mixtures thereof.

In one embodiment, an ester of sterol may be a cholesteryl ester.

Sterols or esters thereof may be selected from the group consisting of cholesterol or its derivatives, ergosterol, desmosterol (3β-hydroxy-5,24-cholestadiene), stigmasterol (stigmasta-5,22-dien-3-ol), lanosterol (8,24-lanostadien-3b-ol), 7-dehydrocholesterol (45,7-cholesterol), dihydrolanosterol (24,25-dihydrolanosterol), zymosterol (5a-cholesta-8,24-dien-3β-ol), lathosterol (5a-cholest-7-en-3β-ol), diosgenin ((3,25R)-spirost-5-en-3-ol), sitosterol (22,23-dihydrostigmasterol), sitostanol, campesterol (campest-5-en-3β-ol), campestanol (5a-campestan-3b-ol), fecosterol, brassicasterol, tomatidine, ursolic acid, 24-methylene cholesterol (5,24(28)-cholestadien-24-methylen-3β-ol), cholesteryl margarate (cholest-5-en-3β-yl heptadecanoate), cholesteryl oleate, cholesteryl stearate, and mixture thereof.

Alternatively, a sterol may be a cholesterol derivative such as an oxidized cholesterol.

Oxidized cholesterols suitable for the disclosure may be 25-hydroxycholesterol, 27-hydroxycholesterol, 20a-hydroxycholesterol, 6-keto-5cx-hydroxycholesterol, 7-keto-cholesterol, 78,25-hydroxycholesterol, 7β-hydroxycholesterol; and combinations thereof. For example, oxidized cholesterols may be 25-hydroxycholesterol and 20a-hydroxycholesterol, and for example it may be 20a-hydroxycholesterol, and mixtures thereof.

In one embodiment, a sterol or an ester thereof may be cholesterol, a cholesteryl ester, or a cholesterol derivative, for example an oxidized cholesterol.

In one embodiment, a sterol may be cholesterol or a cholesteryl ester.

In one embodiment, a sterol may be cholesterol.

In one embodiment, a sterol may be sitosterol.

A corticosteroid may be selected from prednisolone, dexamethasone, prednisone, hydrocortisone, or mixtures thereof.

The structural lipid may be present in a lipid component of a composition or a LNP in a molar amount ranging from about 20 to about 55%, or from about 20% to about 50%, or from about 25% to about 45%, in % w/w relative to the total molar amount of the lipid component.

The structural lipid may be present in a lipid component of a composition or a LNP in a molar amount of about 20%, about 22, about 24, about 26, about 28, about 30, about 32, about 34, about 36, about 38, about 40, about 42, about 44, about 46, about 48, about 50, about 52, about 54, or about 55%, in % w/w relative to the total molar amount of the lipid component.

In one embodiment, a structural lipid may be present in amount of about 28.5%, or about 38.5%, or about 40.9%, or about 42.7%, in % w/w relative to the total molar amount of the lipid components of said LNPs or compositions.

The structural lipid may be present in a lipid component in an amount of 38.5%, in % w/w relative to the total molar amount of the lipid component.

In one embodiment, a structural lipid may be cholesterol.

The structural lipids may be present in a lipid component of compositions or LNPs in a molar ratio lipidic compound: structural lipid, ranging from about 4:1 to about 1:2, for example from about 3.5:1 to about 1:1.8, for example from about 2:1 to about 1:1.5, for example from about 1.5:1 to about 1:1.2, and for example is about 1.3:1 to about 1:1.3.

### PEG-Lipids

The lipid component of the compositions or the LNPs may include a PEG-lipid (or PEGylated lipid).

Contemplated PEG-modified lipids include, but are not limited to, a polyethylene glycol chain of up to 5 kDa in length covalently attached to a lipid with alkyl chain(s) of C₆-C_{w} length. The addition of PEG-modified lipids to a composition of LNPs may prevent complex aggregation and may also provide a means for increasing circulation lifetime and increasing the delivery of the composition or lipid nanoparticles to the target cells.

A suitable PEG-lipid may be, for example, a pegylated diacylglycerol (PEG-DAG), such as λ-(monomethoxy-polyethyleneglycol)-2,3-dimyristoylglycerol (PEG-DMG); a pegylated phosphatidylethanoloamine (PEG-PE); a PEG succinate diacylglycerol (PEG-S-DAG) such as 4—O—(2′,3′-di (tetradecanoyloxy) propyl-2—O—(co-methoxy (polyethoxy) ethyl) butanedioate (PEG-S-DMG); a pegylated ceramide (PEG-cer); DSPC-PEG; DSPE-PEG; a PEG dialkoxypropylcarbamate, such as @-methoxy (polyethoxy) ethyl-N-(2,3-di (tetradecanoxy) propyl) carbamate; 2,3-di (tetradecanoxy) propyl-N-(co-methoxy (polyethoxy) ethyl) carbamate; 2-[(polyethylene glycol)-2000]-N,N-ditetradecylacetamide (ALC-0159); and mixture thereof.

In one embodiment, a suitable PEG-lipid may be selected from the group consisting of PEG-DAG; PEG-DMG; PEG-PE; PEG-S-DAG; PEG-S-DMG; DSPC-PEG; DSPE-PEG; PEG-cer; a PEG-dialkyoxypropylcarbamate; 2-[(polyethylene glycol)-2000]-N,N-ditetradecylacetamide (ALC-0159); and mixture thereof.

For example, a PEG-lipid may be PEG-DMG, PEG-PE, DSPC-PEG, or 2-[(polyethylene glycol)-2000]-N,N-ditetradecylacetamide (ALC-0159), and mixture thereof.

In one embodiment, a PEG-lipid may be a PEG-PE, such as a PEG-2000-PE

In one embodiment, a PEG-lipid may be a PEG-DMG, such as a DMG-PEG-2000.

In one embodiment, a PEG-lipid may be a DSPC-PEG, such as a DSPC-PEG-2000.

In one embodiment, a PEG-lipid may be 2-[(polyethylene glycol)-2000]-N,N-ditetradecylacetamide (ALC-0159).

The PEG-lipid may be present in a lipid component of a composition or a LNP in a molar amount ranging from about 1 to about 15%, from about 1% to about 10%, from about 1% to about 5%, or from about 1% to about 3.5%, in % relative to the total molar amount of the lipid component.

The PEG-lipid may be present in a lipid component of a composition or a LNP in a molar amount of about 1%, about 2, about 3, about 4, about 5, about 6, about 7, about 8, about 9, about 10, about 11, about 12, about 13, about 14, or about 15%, in % relative to the total molar amount of the lipid component.

In one embodiment, a PEG-lipid may be a MDG-PEG, for example a DMG-PEG-2000, for example present in amount of about 1.5%, in % relative to the total molar amount of the lipid component.

In one embodiment, a PEG-lipid may be 2-[(polyethylene glycol)-2000]-N,N-ditetradecylacetamide (ALC-0159), for example present in amount of about 1.6% or about 1.7%, in % w/w relative to the total weight of the lipid components of said LNPs.

A PEG-lipid and the lipidic compound disclosed herein may be present in a lipid component of compositions or LNPs in a molar ratio lipidic compound to PEG-lipid from about 70:1 to about 4:1, from about 40:1 to about 10:1, from about 35:1 to about 15:1, or at about 33:1, or at about 14:1.

### LNPs Formulations

In one embodiment, compositions or LNPs may comprise a lipid component comprising at least a lipidic compound disclosed herein in a molar amount of about 30% to about 70%, a neutral lipid in a molar amount of about 0% to about 50%, a structural lipid in a molar amount of about 20% to about 50%, and optionally a PEG-lipid in a molar amount of about 1% to about 15%, in % relative to the total molar amount of the lipid component.

In one embodiment, compositions or LNPs may comprise a lipid component comprising at least a lipidic compound disclosed herein in a molar amount of about 30% to about 70%, a neutral lipid in a molar amount of about 0% to about 50%, a structural lipid in a molar amount of about 20% to about 50%, and a PEG-lipid in a molar amount of about 1% to about 15%, in % relative to the total molar amount of the lipid component.

In one embodiment, compositions or LNPs may comprise a lipid component comprising a lipidic compound disclosed herein, a neutral lipid, a structural lipid, and a PEG-lipid in a molar amount of about 35% to about 55% of lipidic compound disclosed herein, of about 5% to about 35% of neutral lipid, of about 25% to about 45% of structural lipid, and of about 1.0% to about 2.5% of PEG-lipid, in % relative to the total molar amount of the lipid component.

In one embodiment, compositions or LNPs may comprise a lipid component comprising a lipidic compound disclosed herein, a neutral lipid, a structural lipid, and a PEG-lipid in a molar amount of about 40% to about 50% of lipidic compound, of about 9% to about 30% of neutral lipid, of about 28% to about 45% of structural lipid, and of about 1.5% to about 2.5% of PEG-lipid, in % relative to the total molar amount of the lipid component.

In one embodiment, compositions or LNPs may comprise a lipid component comprising a lipidic compound disclosed herein, a neutral lipid, a structural lipid, and a PEG-lipid in molar ratio of about 35/16/46.5/1.5, of about 50/10/38.5/1.5, of about 57.2/7.1/34.3/1.4, of about 40/15/40/5, of about 50/10/35/4.5/0.5, of about 50/10/35/5, of about 40/10/40/10, of about 35/15/40/10, or of about 52/13/30/5.

In one embodiment, the molar ratio of a lipidic compound disclosed herein, a neutral lipid, a structural lipid, and a PEG-lipid may be of about 35/16/46.5/1.5 or about 50/10/38.5/1.5.

In one embodiment, the molar ratio of a lipidic compound disclosed herein, a neutral lipid, a structural lipid, and a PEG-lipid may be of about 50/10/38.5/1.5.

In one embodiment, compositions or LNPs may comprise a lipid component comprising a lipidic compound disclosed herein, a neutral lipid, a structural lipid, and a PEG-lipid in a molar amount of about 50% of lipidic compound, about 10% of neutral lipid, about 38.5% of structural lipid, and about 1.5% of PEG-lipid, in % relative to the total weight of the lipid components of said LNPs.

In one embodiment, the lipidic compound may be a lipidic compound of formula (I).

In one embodiment, a suitable lipidic compound may be a lipidic compound of formula (II).

In one embodiment, a suitable lipidic compound may be a lipidic compound of formula (IIa).

In one embodiment, a suitable lipidic compound may be a lipidic compound of formula (III).

In one embodiment, a suitable lipidic compound may be a lipidic compound of formula (IV).

In one embodiment, a suitable lipidic compound may be a lipidic compound of formula (V).

In one embodiment, a suitable lipidic compound may be a lipidic compound as indicated in Table 1.

In one embodiment, a suitable lipidic compound may be a lipidic compound as indicated in Table 2.

In some embodiments, a lipidic compound may be of formula (VI), (VII), (VIII), (XI), (XII), (XIV), (XV), (XVI), (XVII), (XVIII), (XIX), (XX), (XXI), (XXX), (XXXI), (XXXII), (XXXIII), (XXXIV), (XXXV), (XXXVI), (XXXVII) or (XXXVIIII).

In some embodiments, a lipidic compound may be of formula (VI), (VII), (XII), (XIV), (XVI), (XVIII), (XIX), (XXI), (XXX), (XXXI), (XXXII), (XXXIII), (XXXIV), (XXXV), (XXXVI), (XXXVII) or (XXXVIIII).

In some embodiments, a lipidic compound may be of formula (VI), (VII), (VIII), (XI), or (XII).

In some embodiments, a lipidic compound may be of formula (VI), (VII), (VIII), or (XII).

In some embodiments, a lipidic compound may be of formula (VII), (XII), (XIV), (XV), (XVI), (XIX), (XX) or (XXI).

In some embodiments, a lipidic compound may be of formula (VII), (XII), (XIV), (XVI), (XIX), or (XXI).

In some embodiments, a lipidic compound may be of formula (VI), (VII) or (XII).

In some embodiments, a lipidic compound may be of formula (XIV), (XVI), (XVIII), (XIX), (XXX), (XXXI), (XXXII), (XXXIII), (XXXIV), (XXXV), (XXXVI), (XXXVII) or (XXXVIIII).

In some embodiments, a lipidic compound may be of formula (XXX), (XXXI), (XXXII), (XXXIII), (XXXIV), (XXXV), (XXXVI), (XXXVII) or (XXXVIIII).

In some embodiments, a lipidic compound may be of formula (XIV), (XVI), (XVIII), (XXX), (XXXII), (XXXIII), (XXXIV), (XXXV), (XXXVI), or (XXXVIIII).

In some embodiments, a lipidic compound may be of formula (XVI), (XVIII), (XXXIII), (XXXIV), or (XXXVIII).

In some embodiments, a lipidic compound may be of formula (XVIII), (XXXIII), (XXXIV), or (XXXVIII).

In some embodiments, a lipidic compound may be of formula (XVIII), (XXXIII), or (XXXVIII).

In one embodiment, the neutral lipid may be DEPE.

In one embodiment, the neutral lipid may be DOPE.

In one embodiment, the neutral lipid may be DSPC.

In one embodiment, the structural lipid may be cholesterol.

In one embodiment, the PEG-lipid may be PEG-PE (PEG-2000-PE) or PEG-DMG (PEG-2000-DMG).

In one embodiment, the PEG-lipid may PEG-DMG (PEG-2000-DMG).

In one embodiment, the lipidic compound may be of formula (VI), (VII), (VIII), (XI), (XII), (XIV), (XV), (XVI), (XVII), (XVIII), (XIX), (XX), (XXI), (XXX), (XXXI), (XXXII), (XXXIII), (XXXIV), (XXXV), (XXXVI), (XXXVII) or (XXXVIIII), the neutral lipid may be DSPC or DOPE, the structural lipid may be cholesterol or sitosterol, and the PEG-lipid may be PEG-DMG (DMG-PEG-2000) or PEG-PE (PE-PEG 2000).

In one embodiment, the lipidic compound may be of formula (VI), (VII), (XII), (XIV), (XVI), (XVIII), (XIX), (XXI), (XXX), (XXXI), (XXXII), (XXXIII), (XXXIV), (XXXV), (XXXVI), (XXXVII) or (XXXVIIII), the neutral lipid may be DSPC or DOPE, the structural lipid may be cholesterol or sitosterol, and the PEG-lipid may be PEG-DMG (DMG-PEG-2000) or PEG-PE (PE-PEG 2000).

In one embodiment, the lipidic compound may be of formula (VI), (VII), (VIII), (XI), or (XII), the neutral lipid may be DSPC or DOPE, the structural lipid may be cholesterol or sitosterol, and the PEG-lipid may be PEG-DMG (DMG-PEG-2000) or PEG-PE (PE-PEG 2000).

In some embodiments, a lipidic compound may be of formula (VI), (VII), (VIII), or (XII), the neutral lipid may be DSPC or DOPE, the structural lipid may be cholesterol or sitosterol, and the PEG-lipid may be PEG-DMG (DMG-PEG-2000) or PEG-PE (PE-PEG 2000).

In one embodiment, the lipidic compound may be of formula (VII), (XII), (XIV), (XV), (XVI), (XIX), (XX) or (XXI), the neutral lipid may be DSPC or DOPE, the structural lipid may be cholesterol or sitosterol, and the PEG-lipid may be PEG-DMG (DMG-PEG-2000) or PEG-PE (PE-PEG 2000).

In some embodiments, a lipidic compound may be of formula (VII), (XII), (XIV), (XVI), (XIX), or (XXI), the neutral lipid may be DSPC or DOPE, the structural lipid may be cholesterol or sitosterol, and the PEG-lipid may be PEG-DMG (DMG-PEG-2000) or PEG-PE (PE-PEG 2000).

In one embodiment, the lipidic compound may be of formula (VI), (VII) or (XII), the neutral lipid may be DSPC or DOPE, the structural lipid may be cholesterol or sitosterol, and the PEG-lipid may be PEG-DMG (DMG-PEG-2000) or PEG-PE (PE-PEG 2000).

In some embodiments, a lipidic compound may be of formula (XIV), (XVI), (XVIII), (XIX), (XXX), (XXXI), (XXXII), (XXXIII), (XXXIV), (XXXV), (XXXVI), (XXXVII) or (XXXVIIII), the neutral lipid may be DSPC or DOPE, the structural lipid may be cholesterol or sitosterol, and the PEG-lipid may be PEG-DMG (DMG-PEG-2000) or PEG-PE (PE-PEG 2000).

In some embodiments, a lipidic compound may be of formula (XXX), (XXXI), (XXXII), (XXXIII), (XXXIV), (XXXV), (XXXVI), (XXXVII) or (XXXVIIII), the neutral lipid may be DSPC or DOPE, the structural lipid may be cholesterol or sitosterol, and the PEG-lipid may be PEG-DMG (DMG-PEG-2000) or PEG-PE (PE-PEG 2000).

In some embodiments, a lipidic compound may be of formula (XIV), (XVI), (XVIII), (XXX), (XXXII), (XXXIII), (XXXIV), (XXXV), (XXXVI), or (XXXVIIII), the neutral lipid may be DSPC or DOPE, the structural lipid may be cholesterol or sitosterol, and the PEG-lipid may be PEG-DMG (DMG-PEG-2000) or PEG-PE (PE-PEG 2000).

In some embodiments, a lipidic compound may be of formula (XVI), (XVIII), (XXXIII), (XXXIV), or (XXXVIII), the neutral lipid may be DSPC or DOPE, the structural lipid may be cholesterol or sitosterol, and the PEG-lipid may be PEG-DMG (DMG-PEG-2000) or PEG-PE (PE-PEG 2000).

In some embodiments, a lipidic compound may be of formula (XVIII), (XXXIII), (XXXIV), or (XXXVIII), the neutral lipid may be DSPC or DOPE, the structural lipid may be cholesterol or sitosterol, and the PEG-lipid may be PEG-DMG (DMG-PEG-2000) or PEG-PE (PE-PEG 2000).

In some embodiments, a lipidic compound may be of formula (XVIII), (XXXIII), or (XXXVIII).

In one embodiment, the lipidic compound may be of formula (VI), the neutral lipid may be DSPC or DOPE, the structural lipid may be cholesterol or sitosterol, and the PEG-lipid may be PEG-DMG (DMG-PEG-2000) or PEG-PE (PE-PEG 2000).

In one embodiment, the lipidic compound may be of formula (VII), the neutral lipid may be DSPC or DOPE, the structural lipid may be cholesterol or sitosterol, and the PEG-lipid may be PEG-DMG (DMG-PEG-2000) or PEG-PE (PE-PEG 2000).

In one embodiment, the lipidic compound may be of formula (VIII), the neutral lipid may be DSPC or DOPE, the structural lipid may be cholesterol or sitosterol, and the PEG-lipid may be PEG-DMG (DMG-PEG-2000) or PEG-PE (PE-PEG 2000).

In one embodiment, the lipidic compound may be of formula (XII), the neutral lipid may be DSPC or DOPE, the structural lipid may be cholesterol or sitosterol, and the PEG-lipid may be PEG-DMG (DMG-PEG-2000) or PEG-PE (PE-PEG 2000).

In one embodiment, the lipidic compound may be of formula (XI), the neutral lipid may be DSPC or DOPE, the structural lipid may be cholesterol or sitosterol, and the PEG-lipid may be PEG-DMG (DMG-PEG-2000) or PEG-PE (PE-PEG 2000).

In one embodiment, the lipidic compound may be of formula (XIV), the neutral lipid may be DSPC or DOPE, the structural lipid may be cholesterol or sitosterol, and the PEG-lipid may be PEG-DMG (DMG-PEG-2000) or PEG-PE (PE-PEG 2000).

In one embodiment, the lipidic compound may be of formula (XV), the neutral lipid may be DSPC or DOPE, the structural lipid may be cholesterol or sitosterol, and the PEG-lipid may be PEG-DMG (DMG-PEG-2000) or PEG-PE (PE-PEG 2000).

In one embodiment, the lipidic compound may be of formula (XVI), the neutral lipid may be DSPC or DOPE, the structural lipid may be cholesterol or sitosterol, and the PEG-lipid may be PEG-DMG (DMG-PEG-2000) or PEG-PE (PE-PEG 2000).

In one embodiment, the lipidic compound may be of formula (XIX), the neutral lipid may be DSPC or DOPE, the structural lipid may be cholesterol or sitosterol, and the PEG-lipid may be PEG-DMG (DMG-PEG-2000) or PEG-PE (PE-PEG 2000).

In one embodiment, the lipidic compound may be of formula (XX), the neutral lipid may be DSPC or DOPE, the structural lipid may be cholesterol or sitosterol, and the PEG-lipid may be PEG-DMG (DMG-PEG-2000) or PEG-PE (PE-PEG 2000).

In one embodiment, the lipidic compound may be of formula (XXI), the neutral lipid may be DSPC or DOPE, the structural lipid may be cholesterol or sitosterol, and the PEG-lipid may be PEG-DMG (DMG-PEG-2000) or PEG-PE (PE-PEG 2000).

In one embodiment, the lipidic compound may be of formula (XXVII), the neutral lipid may be DSPC or DOPE, the structural lipid may be cholesterol or sitosterol, and the PEG-lipid may be PEG-DMG (DMG-PEG-2000) or PEG-PE (PE-PEG 2000).

In one embodiment, the lipidic compound may be of formula (XVII), the neutral lipid may be DSPC or DOPE, the structural lipid may be cholesterol or sitosterol, and the PEG-lipid may be PEG-DMG (DMG-PEG-2000) or PEG-PE (PE-PEG 2000).

In one embodiment, the lipidic compound may be of formula (XVIII), the neutral lipid may be DSPC or DOPE, the structural lipid may be cholesterol or sitosterol, and the PEG-lipid may be PEG-DMG (DMG-PEG-2000) or PEG-PE (PE-PEG 2000).

In one embodiment, the lipidic compound may be of formula (XXX), the neutral lipid may be DSPC or DOPE, the structural lipid may be cholesterol or sitosterol, and the PEG-lipid may be PEG-DMG (DMG-PEG-2000) or PEG-PE (PE-PEG 2000).

In one embodiment, the lipidic compound may be of formula (XXXI), the neutral lipid may be DSPC or DOPE, the structural lipid may be cholesterol or sitosterol, and the PEG-lipid may be PEG-DMG (DMG-PEG-2000) or PEG-PE (PE-PEG 2000).

In one embodiment, the lipidic compound may be of formula (XXXII), the neutral lipid may be DSPC or DOPE, the structural lipid may be cholesterol or sitosterol, and the PEG-lipid may be PEG-DMG (DMG-PEG-2000) or PEG-PE (PE-PEG 2000).

In one embodiment, the lipidic compound may be of formula (XXXIII), the neutral lipid may be DSPC or DOPE, the structural lipid may be cholesterol or sitosterol, and the PEG-lipid may be PEG-DMG (DMG-PEG-2000) or PEG-PE (PE-PEG 2000).

In one embodiment, the lipidic compound may be of formula (XXXIV), the neutral lipid may be DSPC or DOPE, the structural lipid may be cholesterol or sitosterol, and the PEG-lipid may be PEG-DMG (DMG-PEG-2000) or PEG-PE (PE-PEG 2000).

In one embodiment, the lipidic compound may be of formula (XXXV), the neutral lipid may be DSPC or DOPE, the structural lipid may be cholesterol or sitosterol, and the PEG-lipid may be PEG-DMG (DMG-PEG-2000) or PEG-PE (PE-PEG 2000).

In one embodiment, the lipidic compound may be of formula (XXXVI), the neutral lipid may be DSPC or DOPE, the structural lipid may be cholesterol or sitosterol, and the PEG-lipid may be PEG-DMG (DMG-PEG-2000) or PEG-PE (PE-PEG 2000).

In one embodiment, the lipidic compound may be of formula (XXXVII), the neutral lipid may be DSPC or DOPE, the structural lipid may be cholesterol or sitosterol, and the PEG-lipid may be PEG-DMG (DMG-PEG-2000) or PEG-PE (PE-PEG 2000).

In one embodiment, the lipidic compound may be of formula (XXXVIII), the neutral lipid may be DSPC or DOPE, the structural lipid may be cholesterol or sitosterol, and the PEG-lipid may be PEG-DMG (DMG-PEG-2000) or PEG-PE (PE-PEG 2000).

In one embodiment, the lipid component of a composition or a LNP may comprise 50% of lipidic compound of formula (VI), (VII), (VIII), (XI), (XII), (XIV), (XV), (XVI), (XVII), (XVIII), (XIX), (XX), (XXI), (XXX), (XXXI), (XXXII), (XXXIII), (XXXIV), (XXXV), (XXXVI), (XXXVII) or (XXXVIIII), 10% of DSPC or DOPE, 38.5% of cholesterol or sitosterol, and 1.5% of PEG-DMG (PEG-2000-DMG) or PEG-PE (PE-PEG2000), in % relative to the total molar amount of the lipid component.

In one embodiment, the lipid component of a composition or a LNP may comprise 50% of lipidic compound of formula (VI), (VII), (XII), (XIV), (XVI), (XVIII), (XIX), (XXI), (XXX), (XXXI), (XXXII), (XXXIII), (XXXIV), (XXXV), (XXXVI), (XXXVII) or (XXXVIIII), 10% of DSPC or DOPE, 38.5% of cholesterol or sitosterol, and 1.5% of PEG-DMG (PEG-2000-DMG) or PEG-PE (PE-PEG2000), in % relative to the total molar amount of the lipid component.

In one embodiment, the lipid component of a composition or a LNP may comprise 50% of lipidic compound of formula (VI), (VII), (VIII), (XI), or (XII), 10% of DSPC or DOPE, 38.5% of cholesterol or sitosterol, and 1.5% of PEG-DMG (PEG-2000-DMG) or PEG-PE (PE-PEG2000), in % relative to the total molar amount of the lipid component.

In one embodiment, the lipid component of a composition or a LNP may comprise 50% of lipidic compound of formula (VI), (VII), (VIII), or (XII), 10% of DSPC or DOPE, 38.5% of cholesterol or sitosterol, and 1.5% of PEG-DMG (PEG-2000-DMG) or PEG-PE (PE-PEG2000), in % relative to the total molar amount of the lipid component.

In one embodiment, the lipid component of a composition or a LNP may comprise 50% of lipidic compound of formula (VII), (XII), (XIV), (XV), (XVI), (XIX), (XX) or (XXI), 10% of DSPC or DOPE, 38.5% of cholesterol or sitosterol, and 1.5% of PEG-DMG (PEG-2000-DMG) or PEG-PE (PE-PEG2000), in % relative to the total molar amount of the lipid component.

In one embodiment, the lipid component of a composition or a LNP may comprise 50% of lipidic compound of formula (VII), (XII), (XIV), (XVI), (XIX), or (XXI), 10% of DSPC or DOPE, 38.5% of cholesterol or sitosterol, and 1.5% of PEG-DMG (PEG-2000-DMG) or PEG-PE (PE-PEG2000), in % relative to the total molar amount of the lipid component.

In one embodiment, the lipid component of a composition or a LNP may comprise 50% of lipidic compound of formula (VI), (VII) or (XII), 10% of DSPC or DOPE, 38.5% of cholesterol or sitosterol, and 1.5% of PEG-DMG (PEG-2000-DMG) or PEG-PE (PE-PEG2000), in % relative to the total molar amount of the lipid component.

In one embodiment, the lipid component of a composition or a LNP may comprise 50% of lipidic compound of formula (XIV), (XVI), (XVIII), (XIX), (XXX), (XXXI), (XXXII), (XXXIII), (XXXIV), (XXXV), (XXXVI), (XXXVII) or (XXXVIIII), 10% of DSPC or DOPE, 38.5% of cholesterol or sitosterol, and 1.5% of PEG-DMG (PEG-2000-DMG) or PEG-PE (PE-PEG2000), in % relative to the total molar amount of the lipid component.

In one embodiment, the lipid component of a composition or a LNP may comprise 50% of lipidic compound of formula (XXX), (XXXI), (XXXII), (XXXIII), (XXXIV), (XXXV), (XXXVI), (XXXVII) or (XXXVIIII), 10% of DSPC or DOPE, 38.5% of cholesterol or sitosterol, and 1.5% of PEG-DMG (PEG-2000-DMG) or PEG-PE (PE-PEG2000), in % relative to the total molar amount of the lipid component.

In one embodiment, the lipid component of a composition or a LNP may comprise 50% of lipidic compound of formula (XIV), (XVI), (XVIII), (XXX), (XXXII), (XXXIII), (XXXIV), (XXXV), (XXXVI), or (XXXVIIII), 10% of DSPC or DOPE, 38.5% of cholesterol or sitosterol, and 1.5% of PEG-DMG (PEG-2000-DMG) or PEG-PE (PE-PEG2000), in % relative to the total molar amount of the lipid component.

In one embodiment, the lipid component of a composition or a LNP may comprise 50% of lipidic compound of formula (XVI), (XVIII), (XXXIII), (XXXIV), or (XXXVIII), 10% of DSPC or DOPE, 38.5% of cholesterol or sitosterol, and 1.5% of PEG-DMG (PEG-2000-DMG) or PEG-PE (PE-PEG2000), in % relative to the total molar amount of the lipid component.

In one embodiment, the lipid component of a composition or a LNP may comprise 50% of lipidic compound of formula (XVIII), (XXXIII), (XXXIV), or (XXXVIII), 10% of DSPC or DOPE, 38.5% of cholesterol or sitosterol, and 1.5% of PEG-DMG (PEG-2000-DMG) or PEG-PE (PE-PEG2000), in % relative to the total molar amount of the lipid component.

In one embodiment, the lipid component of a composition or a LNP may comprise 50% of lipidic compound of formula (XVIII), (XXXIII), or (XXXVIII), 10% of DSPC or DOPE, 38.5% of cholesterol or sitosterol, and 1.5% of PEG-DMG (PEG-2000-DMG) or PEG-PE (PE-PEG2000), in % relative to the total molar amount of the lipid component.

The neutral lipid may be DSPC.

The structural lipid may be cholesterol.

The PEG-lipid may be PEG-DMG (DMG-PEG-2000).

In one embodiment, the lipidic compound may be of formula (VI), (VII), (VIII), (XI), (XII), (XIV), (XV), (XVI), (XVII), (XVIII), (XIX), (XX), (XXI), (XXX), (XXXII), (XXXIII), (XXXIV), (XXXV), (XXXVI), (XXXVII) or (XXXVIIII), the neutral lipid may be DSPC, the structural lipid may be sitosterol, and the PEG-lipid may be PEG-DMG (DMG-PEG-2000).

In one embodiment, the lipidic compound may be of formula (VI), (VII), (XII), (XIV), (XVI), (XVIII), (XIX), (XXI), (XXX), (XXXI), (XXXII), (XXXIII), (XXXIV), (XXXV), (XXXVI), (XXXVII) or (XXXVIIII), the neutral lipid may be DSPC, the structural lipid may be sitosterol, and the PEG-lipid may be PEG-DMG (DMG-PEG-2000).

In one embodiment, the lipidic compound may be of (VI), (VII), (VIII), (XI), or (XII), the neutral lipid may be DSPC, the structural lipid may be sitosterol, and the PEG-lipid may be PEG-DMG (DMG-PEG-2000).

In one embodiment, the lipidic compound may be of (VI), (VII), (VIII), or (XII), the neutral lipid may be DSPC, the structural lipid may be sitosterol, and the PEG-lipid may be PEG-DMG (DMG-PEG-2000).

In one embodiment, the lipidic compound may be of formula (VII), (XII), (XIV), (XV), (XVI), (XIX), (XX) or (XXI), the neutral lipid may be DSPC, the structural lipid may be sitosterol, and the PEG-lipid may be PEG-DMG (DMG-PEG-2000).

In one embodiment, the lipidic compound may be of formula (VII), (XII), (XIV), (XVI), (XIX), or (XXI), the neutral lipid may be DSPC, the structural lipid may be sitosterol, and the PEG-lipid may be PEG-DMG (DMG-PEG-2000).

In one embodiment, the lipidic compound may be of (VI), (VII) or (XII), the neutral lipid may be DSPC, the structural lipid may be sitosterol, and the PEG-lipid may be PEG-DMG (DMG-PEG-2000).

In one embodiment, the lipidic compound may be of (XIV), (XVI), (XVIII), (XIX), (XXX), (XXXI), (XXXII), (XXXIII), (XXXIV), (XXXV), (XXXVI), (XXXVII) or (XXXVIIII), the neutral lipid may be DSPC, the structural lipid may be sitosterol, and the PEG-lipid may be PEG-DMG (DMG-PEG-2000).

In one embodiment, the lipidic compound may be of (XXX), (XXXI), (XXXII), (XXXIII), (XXXIV), (XXXV), (XXXVI), (XXXVII) or (XXXVIIII), the neutral lipid may be DSPC, the structural lipid may be sitosterol, and the PEG-lipid may be PEG-DMG (DMG-PEG-2000).

In one embodiment, the lipidic compound may be of (XIV), (XVI), (XVIII), (XXX), (XXXI), (XXXII), (XXXIII), (XXXIV), (XXXV), (XXXVI), or (XXXVIIII), the neutral lipid may be DSPC, the structural lipid may be sitosterol, and the PEG-lipid may be PEG-DMG (DMG-PEG-2000).

In one embodiment, the lipidic compound may be of (XVI), (XVIII), (XXXIII), (XXXIV), or (XXXVIII), the neutral lipid may be DSPC, the structural lipid may be sitosterol, and the PEG-lipid may be PEG-DMG (DMG-PEG-2000).

In one embodiment, the lipidic compound may be of (XVIII), (XXXIII), (XXXIV), or (XXXVIII), the neutral lipid may be DSPC, the structural lipid may be sitosterol, and the PEG-lipid may be PEG-DMG (DMG-PEG-2000).

In one embodiment, the lipidic compound may be of (XVIII), (XXXIII), or (XXXVIII), the neutral lipid may be DSPC, the structural lipid may be sitosterol, and the PEG-lipid may be PEG-DMG (DMG-PEG-2000).

### LNPs Manufacturing Methods

Methods for manufacturing LNPs are known in the art.

In one embodiment, LNPs containing a biologically active agent, for example a nucleic acid, may be obtainable by a method comprising at least the steps of:
i) solubilizing, in a water miscible organic solvent, the lipid component of the LNPs,
ii) mixing the organic solvent obtained at step a) with an aqueous solvent containing a biologically active agent, and
iii) obtaining said LNPs, in the aqueous solvent.

In one embodiment, a method for manufacturing LNPs may comprise at least steps of:
i) solubilizing, in a water miscible organic solvent, at least a lipidic compound as disclosed herein, a neutral lipid, a structural lipid, and optionally a PEG-lipid,
ii) mixing the organic solvent obtained at step i) with an aqueous solvent containing a biologically active agent, and
iii) obtaining the lipid nanoparticles containing a biologically active agent, in the aqueous solvent.

The biologically active agent may be a nucleic acid.

Useful water-miscible organic solvents may be any water-miscible organic solvent capable to solubilize the lipidic compound as disclosed herein and any other added lipids. As example of suitable organic solvents, one may cite ethanol or methanol, 1-propanol, isopropanol, t-butanol, THF, DMSO, acetone, acetonitrile, diglyme, DMF, 1-4 dioxane, ethylene glycol, glycerine, hexamethylphosphoramide, hexamethylphosphorous triamide. In one embodiment, the organic solvent may be ethanol and isopropanol.

The lipidic compound as disclosed herein may be present in an amount sufficient to structure the lipid nanoparticles and to encapsulate any payloads to be encapsulated. The amount of lipidic compound to be used may be determined by the skilled person according to any known techniques and is adapted according to the nature and amount of the payload, and nature and amount of other lipids susceptible to be present.

The lipidic compound disclosed herein, the neutral lipid, the structural lipid, and optionally the PEGylated lipid may be present in the organic solvent, respectively, at a molar amount of about 30% to about 70% of lipidic compound, of about 0% to about 50% of neutral lipid, of 20% to about 50% of structural lipid, and of about 1% to about 15% of PEG-lipid, in % relative to the total amount of lipid component.

Aqueous solvents usable at step ii) include aqueous buffered solutions.

As examples of suitable aqueous buffered solution, one may mention acidic buffer, such as include citrate buffer, sodium acetate buffer, succinate buffer, borate buffer or a phosphate buffer. For example, an aqueous buffered solvent may be a citrate buffered solution or an acetate buffered solution.

The pH of the aqueous solvent may range from about 3.5 to about 7.0, for example from about 4.0 to about 6.5, and for example from about 4.5 to about 6.0, and for example may be at about 5.5. In one embodiment, the pH may be of about 4.0.

At step ii), the organic and aqueous solvents may be mixed at a ratio organic solvent: aqueous solvent ranging from about 1:1 to about 1:6. In one embodiment, the ratio may range from about 1:2 to about 1:4, and for example may be a ratio of about 1:3.

According to one embodiment, the organic solvent and the aqueous solvent may be mixed at step ii) at a flow rate ranging from about 0.01 ml/min to about 12 ml/min. In some embodiments, the flow rate may range from about 0.02 ml/min to about 10 ml/min, from about 0.5 ml/min to about 8 ml/min, from about 1 ml/min to about 6 ml/min, or at about 4 ml/min.

The step of mixing may be carried by any known method in the art. For instance, both solvents may be mixed with a T-tube or a Y-connector. Alternatively, the mixing may be carried out by laminar flow mixing with a microfluidic micromixer as described by Belliveau et al. (Mol Ther Nucleic Acids. 2012; 1 (8): e37), the content of which is incorporated by reference.

As indicated, the aqueous solvent at step ii) comprises as a biologically active agent, a nucleic acid. A suitable nucleic acid may be for example as detailed below.

The method may further comprise, if necessary, a step of increasing the pH from acidic to neutral.

In a further embodiment, the method may comprise a step iv) of increasing the pH of the aqueous solvent containing the LNPs obtained at step iii) at a pH ranging from about 5.5 to about 7.5, for example from about 6.0 to about 7.5.

The step of increasing the pH may be carried by any known method in the art. For example, the change in pH may carried by a dialyzing or diafiltration step.

Further, a method for preparing LNPs may comprise any further step suitable to harvest, purify, concentrate and/or sterilize the lipid nanoparticles to further formulate them as a pharmaceutical composition, for example as an immunogenic composition.

The purification may be carried out by dialysis or diafiltration. The dialysis or diafiltration step may be made against an aqueous solvent with a pH ranging from about 5.5 to about 7.5, for example from about 6.0 to about 7.0, for example from about 6.5 to about 7.0, and for example at about 6.5.

Further, if needed, osmolarity may be adjusted to reach a final osmolality close to 290 mOsmol/kg as to inject isotonic solution into the body.

The lipid nanoparticles may be manufactured with a lipidic compound that is of formula (VI), (VII), (VIII), (XI), (XII), (XIV), (XV), (XVI), (XVII), (XVIII), (XIX), (XX), (XXI), (XXX), (XXXII), (XXXIII), (XXXIV), (XXXV), (XXXVI), (XXXVII) or (XXXVIIII), a neutral lipid that is DSPC or DOPE, a structural lipid that is cholesterol, and a PEG-lipid that is PEG-PE (PEG2000-PE) or DMG-PEG (DMG-PEG2000).

The lipid nanoparticles may be manufactured with a lipidic compound that is of formula (VI), (VII), (XII), (XIV), (XVI), (XVIII), (XIX), (XXI), (XXX), (XXXI), (XXXII), (XXXIII), (XXXIV), (XXXV), (XXXVI), (XXXVII) or (XXXVIIII), a neutral lipid that is DSPC or DOPE, a structural lipid that is cholesterol, and a PEG-lipid that is PEG-PE (PEG2000-PE) or DMG-PEG (DMG-PEG2000).

The lipid nanoparticles may be manufactured with a lipidic compound that is of formula (VI), (VII), (VIII), (XI), (XII), (XIV), (XV), (XVI), (XVII), (XVIII), (XIX), (XX), (XXI), (XXX), (XXXII), (XXXIII), (XXXIV), (XXXV), (XXXVI), (XXXVII) or (XXXVIIII), a neutral lipid that is DSPC, a structural lipid that is cholesterol, and a PEG-lipid that is DMG-PEG (DMG-PEG2000).

The lipid nanoparticles may be manufactured with a lipidic compound that is of formula (VI), (VII), (XII), (XIV), (XVI), (XVIII), (XIX), (XXI), (XXX), (XXXI), (XXXII), (XXXIII), (XXXIV), (XXXV), (XXXVI), (XXXVII) or (XXXVIIII), a neutral lipid that is DSPC, a structural lipid that is cholesterol, and a PEG-lipid that is DMG-PEG (DMG-PEG2000).

### Lipid Nanoparticles (LNPs)

The lipid nanoparticles (LNPs) may be characterized by several parameters well-known in the art, such as the mean diameter size, the mode diameter size, the polydispersity index (PI) which reflects the homogeneity of the size distribution of the LNPs, the pKa, and/or the zeta potential which reflects the global surface charge of the LNPs.

The LNPs may be used to encapsulate at least one biologically active agent. The encapsulation rate and the total content of such agent may also be used as parameters characterizing the LNPs.

Mode diameter size, mean diameter size and PI may be measured by Nanoparticles Tracking Analysis (NTA) NS300 from Malvern equipped with a 96 well plate auto-sampler or dynamic light scattering (DLS). pKa may be determined using a fluorescent probe 2-(p-toluidino)-6-napthalene sulfonic acid (TNS). Zeta potential can be determined using electrophoretic mobility or dynamic electrophoretic mobility measurements, for example with the Nicomp 380 ZLS system or the Malvern nanoZS.

The “mean diameter size” of the LNPs may be determined by Nanoparticles Tracking Analysis (NTA) and represents the average diameter of all particles analyzed in the sample. The “mode diameter size” represents the size of the most frequent particles population in number of the sample. Otherwise said, it is the size of the particles with the highest frequency. In regards of the size distribution profile of the sample, the mode diameter size represents the highest point of the peak seen in the distribution.

NTA uses the properties of both Brownian motion and light scattering for obtaining the particle size distribution of samples in a liquid suspension. A laser beam is passed through the sample chamber and the particles in suspension in the beam path scatter light such that they can be seen through a magnification microscope onto which a camera is mounted. The particle movement is captured on a frame-by-frame basis. The center of each of the observed particles is identified and tracked to obtain the average distance moved in the x and y planes. This value helps determine the particle diffusion coefficient (Dt) from which by knowing the sample temperature T and the solvent viscosity n, the sphere-equivalent hydrodynamic diameter d of the particles is determined with the Stokes-Einstein equation:

$$D t = \frac{T {K}_{B}}{3 π η d}$$

where KB is Boltzmann's constant.

The LNPs may have a diameter making them suitable for systemic, for example parenteral, or for intramuscular, intradermic, or subcutaneous administration. Typically, the lipid nanoparticles have a mean average diameter size of less than 600 nanometers (nm), for example of less than 400 nm.

In one embodiment, the LNPs have a mean average diameter size of less than 200 nm. Such size is advantageously compatible with sterile filtration and most appropriate for migration through the lymphatic vessels after intramuscular or subcutaneous administration. This size is also appropriate for intravenous administration, since larger particle injection could induce capillary thrombosis.

In some embodiments, the LNPs may have a mean diameter size in the range of from about 20 nm to about 300 nm, for example from about 25 nm to about 250 nm, for example from about 30 nm to about 200 nm, from about 40 nm to about 180 nm, from about 60 nm to about 170 nm, from about 70 to about 160 nm, and from about 80 to about 150 nm. In one embodiment, the LNPs may have a mean diameter size in the range of about 85 to about 140 nm, as measured by Dynamic Light Scattering (DLS) and Nanoparticles Tracking Analysis (NTA). In a liquid composition (step a) of a method disclosed herein), the LNPs may have a mode diameter size from about 70 nm to about 250 nm, or from about 80 nm to about 200 nm, or from about 85 to about 140 nm, or of about 90 to about 120 nm, as measured by DLS and NTA.

The NTA technique requires that the sample is liquid. Therefore, for the determination of the mean diameter size of the LNPs after the freezing or the freeze-drying step, the obtained frozen or freeze-dried LNPs are thawed or resuspended in a solution, such as an aqueous buffer or water for injection (WFI).

In some embodiments, the lipid nanoparticles may have a Z-average diameter size in the range of from about 20 nm to about 300 nm, for example from about 20 nm to about 250 nm, for example about 30 nm to about 200 nm, about 40 nm to about 180 nm, from about 60 nm to about 170 nm, from about 80 to about 160 nm, from about 90 to about 150 nm, or from about 90 to about 130 nm. In one embodiment, the nanoparticles may have a diameter in the range of about 90 to about 150 nm.

The “Z-average size” of the lipid nanoparticles may be determined by dynamic light scattering (DLS). The Z-Average size or Z-Average mean used in dynamic light scattering is a parameter also known as the cumulants mean. It is the primary and most stable parameter produced by the technique. The Z-Average mean is defined as the ‘harmonic intensity averaged particle diameter’. A Z-average size may be measured with a zeta sizer Nano ZS light scattering instrument (Malvern Instruments). For accurate particle sizing with the Nano ZS, the viscosity of the buffer and the refractive index of the material had to be provided to the equipment software (PBS: v=1.02 cP, RI=1.45).

As minor variations in size may arise during the manufacturing process, a variation up to 20-30% of the stated measurement is acceptable and considered to be within the stated size. Alternatively, size may be determined by filtration screening assays. For example, a particle preparation is less than a stated size, if at least 90%, for example at least 95%, and for example at least 97% of the particles pass through a “screen-type” filter of the stated size.

The “polydispersity index” (PI) is a measurement of the homogeneous or heterogeneous size distribution of the individual lipid nanoparticles in a lipid nanoparticles mixture and indicates the breadth of the particle distribution in a mixture. The PI can be determined, for example, as described herein.

In one embodiment, the polydispersity index of the nanoparticles described herein as measured by dynamic light scattering is 0.5 or less, for example 0.4 or less, for example 0.3 or less, or even for example 0.2 or less. The PI may be in the range of about 0.05 to about 0.2, or in the range of about 0.09 to about 0.17.

In one embodiment, the lipid nanoparticles are colloidally stable in the sense that no, or substantially no, aggregation, precipitation or increase of size and polydispersity index as measured by dynamic light scattering may be observed over a given period of time, e.g., over at least two hours to over several months, for example at least 1, 2, 3, 4, 5, 6 or 12 months.

The lipid nanoparticles as disclosed herein have a pKa ranging from 4.5 to 6.7.

This pKa may be determined using a fluorescent probe 2-(p-toluidino)-6-napthalene sulfonic acid (TNS) and preformed LNPs composed of cationic lipid/DOPE/cholesterol/PEG-lipid (35:16:35:2.5 mol %) in PBS at a concentration of ˜6 mM total lipid. In brief, TNS is prepared as a 100 μM stock solution in distilled water. LNPs are diluted to 100 UM of total lipids in 90 μL of buffered solutions (triplicates) containing 10 mM HEPES, 10 mM 4-morpholineethanesulfonic acid, 10 mM ammonium acetate, 130 mM NaCl, where the pH ranges from 2.71 to 11.5. Ten microliters of stock TNS is added to the LNP solutions and mixed well in a black 96-well plate. Fluorescence intensity is monitored in a Tecan Pro200 plate reader using excitation and emission wavelengths of 321 and 445 nm. With the resulting fluorescence values, a sigmoidal plot of fluorescence versus buffer pH is created. The log of the inflection point of this curve is the apparent pKa of the LNP formulation. Such a method is for example detailed in Semple, S. C. et al. Rational design of cationic lipids for siRNA delivery. Nat. Biotechnol. 28, 172-176 (2010).

The LNPs may contain or encapsulate at least a nucleic acid as a biologically active agent. In such case, the LNPs may have a global surface charge which is the sum of the negative and positive electric charges at the surface of the particles, and which is represented by the zeta potential. The zeta potential is the potential difference between the dispersion medium and the stationary layer of fluid attached to the dispersed particle. Zeta potential is widely used for quantification of the magnitude of the electrical charge at the double layer.

Zeta potential can be calculated using theoretical models and experimentally determined using electrophoretic mobility or dynamic electrophoretic mobility measurements. Electrophoresis may be used for estimating zeta potential of particulates. In practice, the zeta potential of a dispersion can be measured by applying an electric field across the dispersion. Particles within the dispersion with a zeta potential will migrate toward the electrode of opposite charge with a velocity proportional to the magnitude of the zeta potential. This velocity may be measured using the technique of the Laser Doppler Anemometer. The frequency shift or phase shift of an incident laser beam caused by these moving particles may be measured as the particle mobility, and this mobility may be converted to the zeta potential by inputting the dispersant viscosity and dielectric permittivity, and the application of the Smoluchowski theories. Electrophoretic velocity is proportional to electrophoretic mobility, which is the measurable parameter. There are several theories that link electrophoretic mobility with zeta potential.

Suitable systems such as the Nicomp 380 ZLS system or the Malvern nanoZS can be used for determining the zeta potential. Such systems usually measure the electrophoretic mobility and stability of charged particles in liquid suspension. These values are a predictor of the repulsive forces being exerted by the particles in suspension and are directly related to the stability of the colloidal system.

At PH neutral, the zeta potential of the lipid nanoparticles as disclosed herein is close to neutral.

In one advantage, to have a zeta potential close to zero facilitates particle mobility in the body, reduces opsonization and augment access to target tissues

In one embodiment, at pH from 6.0 to 7.5, the zeta potential of the lipid nanoparticles may be from about-3 mV to about ±3 mV, for example from about-1 mV to about ±1 mV, and for example from about-0.5 mV to about ±0.5 mV.

The lipid nanoparticles described herein can be formed by adjusting, at the time of the preparation, a positive to negative charge, depending on the charge ratio of the ionizable lipidic compound as disclosed herein (cationic charges from the quaternary ammonium: N of the terminal radical of formula (I)) to the nucleic acid (anionic charges from the phosphate: P) and mixing the nucleic acid and the lipidic compound. The charges of the ionizable lipidic compound and of the nucleic acid are charges at a selected pH, such as a pH of the formulating process, which is from about 3.0 to about 4.5.

The +/− (N/P) charge ratio of the lipidic compound as disclosed herein to the nucleic acids can be calculated by the following equation. (+/− charge ratio)=[(cationic lipid amount (mol))*(the total number of positive charges in the cationic lipid)]:[(nucleic acid amount (mol))*(the total number of negative charges in nucleic acid)].

The nucleic acid amount and the lipidic compound amount can be easily determined by one skilled in the art in view of a loading amount upon preparation of the nanoparticles.

In one embodiment, the calculated charge ratio of positive charges to negative charges may range from about 1:1 to about 14:1, for example from about 2:1 to about 12:1, for example from about 4:1 to about 10:1, and for example from about 6:1 to about 8:1, and for example is about 6:1.

In one embodiment, the lipid nanoparticles encapsulating a nucleic acid may have a Z-average size of about 110-130 nm, a PI of about 0.15 to about 0.95, and a calculated charge ratio N/P of about 6:1.

### Biologically Active Agents

A biologically active agent may be a prophylactic, a therapeutic or a diagnosing agent.

In some embodiments, a biologically active agent is a nucleic acid.

In some embodiments, a therapeutic agent is a nucleic acid.

A nucleic acid may be or encode a therapeutic agent. In some embodiments, a nucleic acid may be a mRNA encoding a therapeutic agent.

“Therapeutic agent” intends to refer to an active principle proposed to prevent or reduce the risk of occurrence of a disease condition or a symptom of a disease condition or to cure, or reduce the intensity of a disease condition, or to cure or reduce at least one symptom of a disease condition, in individual to whom it is administered. “Individual” intends to refer human and animals.

A therapeutic agent may be a peptide, a protein, a nucleic acid. In some embodiments, a therapeutic agent may be a nucleic acid. A nucleic acid may encode various therapeutic peptides or proteins.

A therapeutic agent may be a genome-editing polypeptide, a chemokine, a cytokine, a growth factor, an antibody, an enzyme, a structural protein, a blood protein, a hormone, a transcription factor, or an antigen.

In one embodiment, a therapeutic agent may be a genome-editing polypeptide. In some embodiments, the genome-editing polypeptide is a CRISPR protein, such as CRISPR/Cas9, a restriction nuclease, a meganuclease, a transcription activator-like effector protein (TALE, including a TALE nuclease, TALEN), or a zinc finger protein (ZF, including a ZF nuclease, ZFN). See, e.g., Int'l Pub. No. WO 2020/139783.

A therapeutic agent may be a cytokine or a chemokine suitable for stimulating or inhibiting an immune response, stimulating or preventing cell growth, or reducing an inflammation. Examples of suitable cytokine or chemokine include, but are not limited to, insulin, insulin-like growth factor, human growth hormone (hGH), tissue plasminogen activator (tPA), cytokines, such as interleukins (IL), e.g., IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, interferon (IFN) alpha, IFN beta, IFN gamma, IFN omega or IFN tau, tumor necrosis factor (TNF), such as TNF alpha and TNF beta, TNF gamma, TNF-related apoptosis-inducing ligand (TRAIL); lymphotoxin-β (LT-B), granulocyte colony-stimulating factor (G-CSF), granulocyte-macrophage colony-stimulating factor (GM-CSF), macrophage colony-stimulating factor (M-CSF), monocyte chemotactic protein-1 (MCP-1), and growth factors, such as vascular endothelial growth factor (VEGF). Also included is the production of erythropoietin or any other hormone growth factors. The cytokine or the chemokine may be encoded in a nucleic acid.

In some embodiments, a therapeutic agent may be an antibody. As used herein, the term “antibody” refers to a whole antibody comprising two light chain polypeptides and two heavy chain polypeptides, or an antigen-binding fragment thereof. An antibody may be a monoclonal antibody (e.g., full length monoclonal antibody) that displays a single binding specificity and affinity for a particular epitope. An antigen-binding fragment may be a single chain antibody, a single chain Fv fragment (scFv), an Fd fragment, a Fab fragment, an Fab′ fragment, or an F(ab′)₂ fragment. An antibody may recognize a tumor antigen or an infectious disease antigen, against which a protective or a therapeutic immune response is desired, e.g., antigens expressed by a tumor cell. Examples of antibodies include, for example, adalimumab, infliximab, rituximab, ipilimumab, tocilizumab, canakinumab, itolizumab, or tralokinumab. The antibody may be encoded in a nucleic acid.

In some embodiments, a therapeutic peptide or protein may be an enzyme with desirable uses for modulating metabolism or growth in a subject. In some embodiments, an enzyme may be administered to replace an endogenous enzyme that is absent or dysfunctional. In some embodiments, an enzyme may be used to treat a metabolic storage disease. A metabolic storage disease results from the systemic accumulation of metabolites due to the absence or dysfunction of an endogenous enzyme. Such metabolites include lipids, glycoproteins, and mucopolysaccharides. Examples of enzyme replacement therapy include lysosomal diseases, such as Gaucher disease, Fabry disease, MPS I, MPS Il (Hunter syndrome), MPS VI and Glycogen storage disease type II. A therapeutic peptide or protein may be encoded in a nucleic acid.

A structural protein may be, for example, collagen, fibroin, fibrinogen, elastin, tubulin, actin, and myosin. A structural protein may be encoded in a nucleic acid.

A blood protein may be, for example, thrombin, serum albumin, Factor VII, insulin, Factor IX, Factor X, tissue plasminogen activator, protein C, von Willebrand factor, antithrombin III, glucocerebrosidase, erythropoietin granulocyte colony stimulating factor (GCSF) or anticoagulants, and the like. A blood protein may be encoded in a nucleic acid.

A hormone may be for example insulin, thyroid hormone, gonadotrophin, trophic hormones, prolactin, oxytocin, dopamine, bovine somatotropin, leptins and the like. A hormone may be encoded in a nucleic acid.

Transcription factors (TFs) recognize specific DNA sequences to control chromatin and transcription, forming a complex system that guides expression of the genome. Several families of transcription factors exist, and members of each family may share structural characteristics. As example of transcription factors, one may cite helix-turn-helix (e.g., Oct-1), helix-loop-helix (e.g., E2A), zinc finger (e.g., glucocorticoid receptors, GATA proteins), basic protein-leucine zipper [cyclic AMP response element-binding factor (CREB), activator protein-1 (AP-1)], or β-sheet motifs [e.g. nuclear factor-κB (NF-κB)]. Transcription factors may be encoded in a nucleic acid.

A therapeutic agent may be an antigen suitable for triggering an immune response, for example in cancer therapy or in a treatment of an infectious disease (e.g., a viral, bacterial, fungal, protozoal or parasitic infection. An antigen may be encoded in a nucleic acid.

According to some embodiments, compositions containing LNPs as disclosed herein which comprise antigens may therefore be immunogenic or vaccine compositions.

Antigen-containing compositions may vary in their valency. Valency refers to the number of antigenic components in the composition. The immunogenic or vaccine compositions may be monovalent or multivalent, i.e., divalent, trivalent compositions, or more. Multivalent compositions may comprise 2, 3, 4, 5, 6, 7, 8, 9, 10, 11,12,13,14, 15, 16, 17, 18, 19, 20, or more antigens or antigenic moieties (e.g., antigenic peptides, etc.). The antigenic components may be on a single polynucleotide or on separate polynucleotides.

Compositions as disclosed herein may be used to protect, treat, or cure infection arising from contact with an infectious agent, such as bacteria, viruses, fungi, protozoa, and parasites.

Compositions as disclosed herein may be used to protect, treat, or cure cancer diseases.

A nucleic acid may encode for at least one antigen.

According to some embodiments, a nucleic acid may encode for at least one antigen selected in the group consisting of bacterial antigens, viral antigens, and tumour antigens.

### Bacterial Antigens

The bacterium can be a Gram-positive bacterium or a Gram-negative bacterium. Bacterial antigens may be obtained from Acinetobacter baumannii, Bacillus anthracis, Bacillus subtilis, Bordetella pertussis, Borrelia burgdorferi, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, coagulase Negative Staphylococcus, Corynebacterium diphtheria, Enterococcus faecalis, Enterococcus faecium, Escherichia coli, enterotoxigenic Escherichia coli (ETEC), enteropathogenic E. coli, E. coli 0157: H7, Enterobacter sp., Francisella tularensis, Haemophilus influenzae, Helicobacter pylori, Klebsiella pneumoniae, Legionella pneumophila, Leptospira interrogans, Listeria monocytogenes, Moraxella catarralis, Mycobacterium leprae, Mycobacterium tuberculosis, Mycoplasma pneumoniae, Neisseria gonorrhoeae, Neisseria meningitides, Proteus mirabilis, Proteus sps., Pseudomonas aeruginosa, Rickettsia rickettsii, Salmonella typhi, Salmonella typhimurium, Serratia marcesens, Shigella flexneri, Shigella sonnei, Staphylococcus aureus, Staphylococcus epidermidis, Staphylococcus saprophyticus, Streptococcus agalactiae, Streptococcus mutans, Streptococcus pneumoniae, Streptococcus pyogenes, Treponema pallidum, Vibrio cholerae, and Yersinia pestis.

### Viral Antigens

Viral antigens may be obtained from adenovirus; Herpes simplex, type 1; Herpes simplex, type 2; encephalitis virus, papillomavirus, Varicella-zoster virus; Epstein-barr virus; Human cytomegalovirus; Human herpesvirus, type 8; Human papillomavirus; BK virus; JC virus; Smallpox; polio virus, Hepatitis B virus; Human bocavirus; Parvovirus B19; Human astrovirus; Norwalk virus; coxsackievirus; hepatitis A virus; poliovirus; rhinovirus; Severe acute respiratory syndrome virus; Hepatitis C virus; yellow fever virus; dengue virus; West Nile virus; Rubella virus; Hepatitis E virus; Human immunodeficiency virus (HIV); Influenza virus, type A or B; Guanarito virus; Junin virus; Lassa virus; Machupo virus; Sabia virus; Crimean-Congo hemorrhagic fever virus; Ebola virus; Marburg virus; Measles virus; Mumps virus; Parainfluenza virus; Respiratory syncytial virus (RSV); Human metapneumovirus; Hendra virus; Nipah virus; Rabies virus; Hepatitis D; Rotavirus; Orbivirus; Coltivirus; Hantavirus, Middle East Respiratory Coronavirus; SARS-Cov-2 virus; Chikungunya virus; Zika virus; parainfluenza virus; Human Enterovirus; Hanta virus; Japanese encephalitis virus; Vesicular exanthernavirus; Eastern equine encephalitisor; or Banna virus.

In one embodiment, the antigen is from a strain of Influenza A or Influenza B virus or combinations thereof. The strain of Influenza A or Influenza B may be associated with birds, pigs, horses, dogs, humans or non-human primates.

The nucleic acid may encode a hemagglutinin protein or a fragment thereof. The hemagglutinin protein may be H1, H2, H3, H4, H5, H6, H7, H8, H9, H10, H11, H12, H13, H14, H15, H16, H17, H18, or a fragment thereof. The hemagglutinin protein may or may not comprise a head domain (HA1). Alternatively, the hemagglutinin protein may or may not comprise a cytoplasmic domain.

In embodiments, the hemagglutinin protein is a truncated hemagglutinin protein. The truncated hemagglutinin protein may comprise a portion of the transmembrane domain.

In some embodiments, the virus may be selected from the group consisting of H1N1, H3N2, H7N9, H5N1 and H10N8 virus or a B strain virus.

In another embodiment, the antigen may be from a Respiratory syncytial virus (RSV). Suitable RSV antigens may be from RSV A and/or RSV B strains. RSV antigens may be, for example, the fusion glycoprotein F protein, or the adhesion protein G protein.

In another embodiment, the antigen may be from a coronavirus such as SARS-Cov-1 virus, SARS-Cov-2 virus, or MERS-Cov virus. In some embodiments, an antigen may be a SARS-Cov2 antigen, such as a spike protein from SARS-Cov2.

### Tumour Antigens

An antigen may be a tumor antigen, i.e., a constituent of cancer cells such as a protein or a peptide expressed in a cancer cell. The term “tumor antigen” relates to proteins that are under normal conditions specifically expressed in a limited number of tissues and/or organs or in specific developmental stages and are expressed or aberrantly expressed in at least one tumor or cancer tissue. Tumor antigens include, for example, differentiation antigens, for example cell type specific differentiation antigens, i.e., proteins that are under normal conditions specifically expressed in a certain cell type at a certain differentiation stage and germ line specific antigens. For example, a tumor antigen is presented by a cancer cell in which it is expressed.

For example, tumor antigens may include the carcinoembryonal antigen, a 1-fetoprotein, isoferritin, and fetal sulphoglycoprotein, cc2-H-ferroprotein and γ-fetoprotein.

Other examples for tumor antigens that may be useful in the present disclosure are p53, ART-4, BAGE, beta-catenin/m, Bcr-abL CAMEL, CAP-1, CASP-8, CDC27/m, CD 4/m, CEA, the cell surface proteins of the claudin family, such as CLAUDIN-6, CLAUDIN-18.2 and CLAUDIN-12, c-MYC, CT, Cyp-B, DAM, ELF2M, ETV6-AML1, G250, GAGE, GnT-V, Gapl OO, HAGE, HER-2/neu, HPV-E7, HPV-E6, HAST-2, hTERT (or hTRT), LAGE, LDLR/FUT, MAGE-A, for example MAGE-A1, MAGE-A2, MAGE-A3, MAGE-A4, MAGE-A5, MAGE-A6, MAGE-A7, MAGE-A8, MAGE-A9, MAGE-A10, MAGE-A11, or MAGE-A12, MAGE-B, MAGE-C, MART-1/Melan-A, MC1 R, Myosin/m, MUC1, MUM-1, -2, -3, NA88-A, NF1, NY-ESO-1, NY-BR-1, pl 90 minor BCR-abL, Pm I/RARa, PRAME, proteinase 3, PSA, PSM, RAGE, RUI or RU2, SAGE, SART-1 or SART-3, SCGB3A2, SCP 1, SCP2, SCP3, SSX, SURVIVIN, TEL/AMLI, TPI/m, TRP-1, TRP-2, TRP-2/1NT2, TPTE and WT, for example WT-1.

### Adjuvants

Compositions or LNPs containing a nucleic acid encoding for an antigen may further comprise, or may be co-administered with, an adjuvant or an immune potentiator.

Adjuvants may include, but are not limited to, natural or synthetic adjuvants. They may be organic or inorganic.

Adjuvants may be selected from any of the classes (1) mineral salts, e.g., aluminium hydroxide and aluminium or calcium phosphate gels; (2) emulsions including: oil emulsions and surfactant based formulations, e.g., microfluidised detergent stabilised oil-in-water emulsion, purified saponin, oil-in-water emulsion, stabilised water-in-oil emulsion; (3) particulate adjuvants, e.g., virosomes (unilamellar liposomal vehicles incorporating influenza haemagglutinin), structured complex of saponins and lipids, polylactide co-glycolide (PLG); (4) microbial derivatives; (5) endogenous human immunomodulators; and/or (6) inert vehicles, such as gold particles; (7) microorganism derived adjuvants; (8) tensioactive compounds; (9) carbohydrates; or combinations thereof.

Selection of appropriate adjuvants and appropriate amount of adjuvant will be evident to one of ordinary skill in the art.

Specific adjuvants may include, without limitation, cationic liposome-DNA complex JVRS-100, aluminum hydroxide vaccine adjuvant, aluminum phosphate vaccine adjuvant, aluminum potassium sulfate adjuvant, alhydrogel, ISCOM(s)™, Freund's Complete Adjuvant, Freund's Incomplete Adjuvant, CpG DNA Vaccine Adjuvant, Cholera toxin, Cholera toxin B subunit, Liposomes, Saponin Vaccine Adjuvant, DDA Adjuvant, Squalene-based Adjuvants, Etx B subunit Adjuvant, IL-12 Vaccine Adjuvant, LTK63 Vaccine Mutant Adjuvant, TiterMax Gold Adjuvant, Ribi Vaccine Adjuvant, Montanide ISA 720 Adjuvant, Corynebacterium-derb/ed P40 Vaccine Adjuvant, MPL™ Adjuvant, AS04, AS02, AS01, Lipopolysaccharide Vaccine Adjuvant, Muramyl Dipeptide Adjuvant, CRL1005, Killed Corynebacterium parvum Vaccine Adjuvant, Montanide ISA 51, Bordetella pertussis component Vaccine Adjuvant, Cationic Liposomal Vaccine Adjuvant, Adamantylamide Dipeptide Vaccine Adjuvant, Arlacel A, VSA-3 Adjuvant, Aluminum vaccine adjuvant, Polygen Vaccine Adjuvant, Adjumer™, Algal Glucan, Bay R1005, Theramide®, Stearyl Tyrosine, Specol, Algammulin, Avridine®, Calcium Phosphate Gel, CTA1-DD gene fusion protein, DOC/Alum Complex, Gamma Inulin, Gerbu Adjuvant, GM-CSF, GMDP, Recombinant hIFN-gamma/Interferon-g, Interleukin-iβ, Interleukin-2, Interleukin-7, Sclavo peptide, Rehydragel LV, Rehydragel HPA, Loxoribine, MF59, MTP-PE Liposomes, Murametide, Murapalmitine, D-Murapalmitine, NAGO, Non-Ionic Surfactant Vesicles, PMMA, PAA, Protein Cochleates, QS-21, SPT (Antigen Formulation), nanoemulsion vaccine adjuvant, AS03, Quil-A vaccine adjuvant, RC529 vaccine adjuvant, LTR192G Vaccine Adjuvant, E. coli heat-labile toxin, LT, amorphous aluminum hydroxyphosphate sulfate adjuvant, Calcium phosphate vaccine adjuvant, Montanide Incomplete Seppic Adjuvant, Imiquimod, Resiquimod, AF03, Flagellin, Poly (LC), ISCOMATRIX®, Abisco-100 vaccine adjuvant, Albumin-heparin microparticles vaccine adjuvant, AS-2 vaccine adjuvant, B7-2 vaccine adjuvant, DHEA vaccine adjuvant, Immunoliposomes Containing Antibodies to Costimulatory Molecules, SAF-1, Sendai Proteoliposomes, Sendai-containing Lipid Matrices, Threonyl muramyl dipeptide (TMDP), Ty Particles vaccine adjuvant, Bupivacaine vaccine adjuvant, DL-PGL (Polyester poly (DL-lactide-co-glycolide)) vaccine adjuvant, IL-15 vaccine adjuvant, LTK72 vaccine adjuvant, MPL-SE vaccine adjuvant, non-toxic mutant El 12K of Cholera Toxin mCT-EI 12K, and/or Matrix-S.

### Protein Expression

The compositions or the LNPs comprising or encapsulating a nucleic acid encoding for a protein may be used for treating individuals deficient in said protein. Therefore, the compositions or the LNPs may be used in methods for treating individuals deficient in a protein comprising administering the compositions or the LNPs comprising at least one nucleic acid, for example an mRNA, wherein the nucleic acid encodes a functional protein corresponding to the protein which is deficient in the individual. In embodiments, following expression of the nucleic acid by a target cell a functional protein is produced.

The disclosure also relates to methods of intracellular delivery of nucleic acids that are capable of correcting existing genetic defects and/or providing beneficial functions to at least one target cell. Following successful delivery to target tissues and cells, the compositions and nucleic acids transfect that target cell and the nucleic acids (e.g., mRNA) can be translated into the gene product of interest (e.g., a functional protein or enzyme) or can otherwise modulate or regulate the presence or expression of the gene product of interest.

The compositions and methods provided herein are useful in the management and treatment of a large number of diseases, for example diseases which result from protein and/or enzyme deficiencies. Individuals suffering from such diseases may have underlying genetic defects that lead to the compromised expression of a protein or enzyme, including, for example, the non-synthesis of the protein, the reduced synthesis of the protein, or synthesis of a protein lacking or having diminished biological activity.

Alternatively, the nucleic acids may encode full length antibodies or smaller antibodies (e.g., both heavy and light chains) to confer immunity to a subject. In an alternative embodiment the compositions of the present disclosure encode antibodies that may be used to transiently or chronically effect a functional response in subjects. For example, the mRNA nucleic acids of the present disclosure may encode a functional monoclonal or polyclonal antibody, which upon translation (and as applicable, systemic excretion from the target cells) may be useful for targeting and/or inactivating a biological target (e.g., a stimulatory cytokine such as tumor necrosis factor). Similarly, the mRNA nucleic acids of the present disclosure may encode, for example, functional anti-nephritic factor antibodies useful for the treatment of membranoproliferative glomerulonephritis type II or acute hemolytic uremic syndrome, or alternatively may encode anti-vascular endothelial growth factor (VEGF) antibodies useful for the treatment of VEGF-mediated diseases, such as cancer.

Alternatively, compositions or LNPs may comprise or encapsulate a nucleic acid encoding or representing a RNAi able to reduce or prevent the expression of a protein for treating individuals suffering from an excess of expression of said protein.

### Nucleic Acids

A nucleic acid suitable for the present disclosure may be a deoxyribonucleic acid (DNA) or a ribonucleic acid (RNA). Nucleic acid includes genomic DNA, cDNA, mRNA, recombinantly produced and chemically synthesized molecules.

A nucleic acid may be a single-stranded or a double-stranded molecule, linear or closed covalently to form a circle. A nucleic acid may be a double stranded RNA (dsRNA); a single stranded RNA (ssRNA); a double stranded DNA (dsDNA); a single stranded DNA (ssDNA); and combinations thereof.

Compositions or LNPs containing a nucleic may be employed for introduction into, i.e., transfection of, cells, of the nucleic acid, for example, for recombinant protein expression, for gene replacement, for suppressing or increasing expression of a host protein.

A nucleic acid may be of eukaryotic or prokaryotic origin, and for example of human, animal, plant, bacterial, yeast or viral origin and the like. It may be obtained by any technique known to persons skilled in the art and for example by screening libraries, by chemical synthesis or alternatively by mixed methods including chemical or enzymatic modification of sequences obtained by screening libraries. It may be chemically modified.

A nucleic acid may be comprised in a vector. Vectors are known to the skilled person and may include plasmid vectors, cosmid vectors, phage vectors such as lambda phage, viral vectors such as adenoviral or baculoviral vectors, or artificial chromosome vectors such as bacterial artificial chromosomes (BAC), yeast artificial chromosomes (YAC), or PI artificial chromosomes (PAC). Vectors include expression as well as cloning vectors. Expression vectors comprise plasmids as well as viral vectors and generally contain a desired coding sequence and appropriate DNA sequences necessary for the expression of the operably linked coding sequence in a specific host organism (e.g., bacteria, yeast, plant, insect, or mammal) or in in vitro expression systems. Cloning vectors are generally used to engineer and amplify a certain desired DNA fragment and may lack functional sequences needed for expression of the desired DNA fragments.

A nucleic acid may be a messenger RNA (mRNA); a microRNA (miRNA); a short (or small) interference RNA (siRNA); small hairpin RNA (shRNA); a long non-coding RNA (lncRNA); an asymmetrical interfering RNA (aiRNA); a self-amplifying RNA (saRNA); a small nuclear RNA (snRNA); a small nucleolar RNA (snoRNA); a guide RNA (gRNA); an anti-sense oligonucleotide (ASO); a plasmid DNA (pDNA); closed-ended DNA (ceDNA), and combinations thereof.

In some embodiments, a nucleic acid may be a RNA.

In some embodiments, a nucleic acid may be a messenger RNA (mRNA); a microRNA (miRNA); a short (or small) interference RNA (siRNA); small hairpin RNA (shRNA); a long non-coding RNA (lncRNA); an asymmetrical interfering RNA (aiRNA); a self-amplifying RNA (saRNA); a guide RNA (gRNA); and combinations thereof.

In some embodiments, LNPs may contain as nucleic acids a mRNA encoding for a CRISPR protein, such as CRISPR/Cas9, and a guide RNA (gRNA). A gRNA may be provided as rRNA: tracrRNA duplex or as a single guide RNA (sgRNA). In some embodiments, a CRISPR protein may be provided directly as a polypeptide and not as an mRNA encoding for a CRISPR protein.

In some embodiments, a RNA may be a messenger RNA (mRNA).

In some embodiments, a nucleic acid may encode a genome-editing polypeptide, a chemokine, a cytokine, a growth factor, an antibody, an enzyme, a structural protein, a blood protein, a hormone, a transcription factor, or an antigen, such as described herein.

Messenger RNA (mRNA)

mRNA is typically thought of as the type of RNA that carries information from DNA to the ribosome. The existence of mRNA is typically very brief and includes processing and translation, followed by degradation. Typically, in eukaryotic organisms, mRNA processing comprises the addition of a “cap” on the N-terminal (5′) end, and a “tail” on the C-terminal (3′) end.

A typical cap is a 7-methylguanosine cap, which is a guanosine that is linked through a 5′-5′-triphosphate bond to the first transcribed nucleotide. The presence of the cap is important in providing resistance to nucleases found in most eukaryotic cells. A 5′ cap is typically added as follows: first, a RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase.

The tail is typically a polyadenylation event whereby a polyadenylyl moiety is added to the 3′ end of the mRNA molecule. The presence of this “tail” serves to protect the mRNA from exonuclease degradation. Messenger RNA is translated by the ribosomes into a series of amino acids that make up a protein.

In some embodiments, mRNAs include a 5′ and/or 3′ untranslated region (UTR). In some embodiments, mRNA disclosed herein comprise a 5′ UTR that includes one or more elements that affect an mRNA's stability or translation. In some embodiments, a 5′ UTR may be between about 50 and 500 nucleotides in length. In some embodiments, mRNA disclosed herein comprise a 3′ UTR comprising one or more of a polyadenylation signal, a binding site for proteins that affect a mRNA's stability of location in a cell, or one or more binding sites for miRNAs. In some embodiments, a 3′ UTR may be between 50 and 500 nucleotides in length or longer. In some embodiments, the mRNAs disclosed herein comprise a 5′ or 3′ UTR that is derived from a gene distinct from the one encoded by the mRNA transcript. In some embodiments, the mRNAs disclosed herein comprise a 5′ or 3′ UTR that is chimeric.

The mRNAs disclosed herein may be synthesized according to any of a variety of known methods. For example, mRNAs according to the present disclosure may be synthesized via in vitro transcription (IVT). Methods for in vitro transcription are known in the art. See, e.g., Geall et al. (2013) Semin. Immunol. 25 (2): 152-159; Brunelle et al. (2013) Methods Enzymol. 530:101-14, the content of which is incorporated by reference. Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7 or SP6 RNA polymerase), DNAse I, pyrophosphatase, and/or RNAse inhibitor. The exact conditions will vary according to the specific application. The presence of these reagents is undesirable in a final mRNA product and are considered impurities or contaminants which must be purified to provide a clean and homogeneous mRNA that is suitable for therapeutic use. While mRNA provided from in vitro transcription reactions may be desirable in some embodiments, other sources of mRNA can be used according to the instant disclosure including wild-type mRNA produced from bacteria, fungi, plants, and/or animals.

The mRNA disclosed herein may be modified or unmodified. In some embodiments, the mRNA disclosed herein contain one or more modifications that typically enhance RNA stability. Exemplary modifications include backbone modifications, sugar modifications, or base modifications. In some embodiments, the disclosed mRNAs may be synthesized from naturally occurring nucleotides and/or nucleotide analogues (modified nucleotides) including, but not limited to, purines (adenine (A), guanine (G)) or pyrimidines (thymine (T), cytosine (C), uracil (U)), and as modified nucleotides analogues or derivatives of purines and pyrimidines, such as e.g. 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxy acetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, 3-D-mannosyl-queosine, phosphoramidates, phosphorothioates, peptide nucleotides, methylphosphonates, 7-deazaguanosine, 5-methylcytosine, and inosine. In some embodiments, the disclosed mRNAs comprise at least one chemical modification including but not limited to, consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′—O-methyl uridine. In some embodiments, the modified nucleotides comprise N1-methylpseudouridine. The preparation of such analogues is known to a person skilled in the art e.g., from the U.S. Pat. Nos. 4,373,071, 4,401,796, 4,415,732, 4,458,066, 4,500,707, 4,668,777, 4,973,679, 5,047,524, 5,132,418, 5,153,319, 5,262,530, and 5,700,642, the content of which is incorporated by reference.

The term “RNA” relates to a molecule which comprises ribonucleotide residues and for example being entirely or substantially composed of ribonucleotide residues. “Ribonucleotide” relates to a nucleotide with a hydroxyl group at the 2′-position of a B-D-ribofuranosyl group. It includes double stranded RNA, single stranded RNA, isolated RNA such as partially purified RNA, essentially pure RNA, synthetic RNA, or recombinantly produced RNA.

For the sake of clarity, a mRNA encompasses any coding RNA molecule, which may be translated by a eukaryotic host into a protein. A coding RNA molecule generally refers to a RNA molecule comprising a sequence coding for a protein of interest, and which may be translated by the eukaryotic host, said sequence starting with a start codon (ATG) and for example terminated by a stop codon (i.e. TAA, TAG. TGA).

A RNA may be a naturally occurring RNA or a modified RNA that differs from naturally occurring RNA by the addition, deletion, substitution and/or alteration of at least one nucleotide. Such alterations can include addition of non-nucleotide material, such as to the end(s) of a RNA or internally, for example at least one nucleotide of the RNA. Nucleotides in RNA molecules can also comprise non-standard nucleotides, such as non-naturally occurring nucleotides or chemically synthesized nucleotides or deoxynucleotides. These altered RNAs can be referred to as analogs or analogs of naturally occurring RNA.

A mRNA may be produced by in vitro transcription using a DNA template. Alternatively, the RNA may be obtained by chemical synthesis. Such methods are known to the skilled person. For example, there is a variety of in vitro transcription kits commercially available.

A RNA may be in vitro synthesized in a cell-free system, using appropriate cell extracts and an appropriate DNA template. For example, cloning vectors are applied for the generation of transcripts. The promoter for controlling transcription can be any promoter for any RNA polymerase. Some examples of RNA polymerases are the T7, T3, and SP6 RNA polymerases. A DNA template for in vitro transcription may be obtained by cloning of a nucleic acid, for example a cDNA, and introducing it into an appropriate vector for in vitro transcription. The cDNA may be obtained by reverse transcription of RNA. For example, cloning vectors are used for producing transcripts which generally are designated transcription vectors.

A RNA may encode for a protein or a peptide. That is, if present in the appropriate environment, for example within a cell, such as an antigen-presenting cell, for example a dendritic cell, the RNA can be expressed to produce a protein or peptide it encodes. The stability and translation efficiency of an RNA may be modified as required.

In some embodiments, a mRNA may encode a genome-editing polypeptide, a chemokine, a cytokine, a growth factor, an antibody, an enzyme, a structural protein, a blood protein, an hormone, a transcription factor, or an antigen, such as described herein.

In some embodiments, a mRNA may encode for an antigen.

RNA molecules may be of variable length. Thus, they may be short RNA molecules, for instance RNA molecules shorter than about 100 nucleotides, or long RNA molecules, for instance longer than about 100 nucleotides, or even longer than about 300 nucleotides.

A mRNA may be at least 30 nucleotides in length.

A mRNA may comprise a 5′Cap structure, a 5′-UTR sequence, an ORF sequence coding for a protein or a peptide, a 3′-UTR sequence, and a poly (A) tail.

Typically, a mRNA may comprise or consist of the following general formula:
[5′Cap] w-[5′UTR] x-[Gene of Interest]-[3′UTR] y-[PolyA] z
wherein [5′Cap] contains a methyl guanine nucleotide linked to mRNA via a 5′ to 5′ linkage, wherein [5′UTR] and [3′UTR] are untranslated regions (UTR),
wherein [5′UTR] contains a Kozak sequence,
wherein [Gene of Interest] is any gene coding for a protein of interest,
wherein [PolyA] is a poly (A) tail, and
wherein w, x, y, and z, are identical or different, and equal to 0 or 1.

A Kozak sequence refers to a sequence, which is generally a consensus sequence, occurring in eukaryotic mRNAs and which plays a major role in the initiation of the translation process. Kozak sequences and Kozak consensus sequences are well known in the art.

The [3′UTR] does not express any proteins. The purpose of the [3′UTR] is to increase the stability of the mRNA. According to a one embodiment, the a-globin UTR is chosen because it is known to be devoid of instability.

A sequence corresponding to the gene of interest may be codon-optimized in-order-to obtain a satisfactory protein production within the host which is considered.

A poly (A) tail consists of multiple adenosine monophosphates that is well known in the art. A poly (A) tail is generally produced during a step called polyadenylation that is one of the post-translation modifications which generally occur during the production of mature messenger RNAs. Such poly (A) tail contributes to the stability and the half-life of the mRNA and can be of variable length. For example, a poly (A) tail may be equal or longer than 10 A nucleotides, which includes equal or longer than 20 A nucleotides, which includes equal or longer than 100 A nucleotides, and for example about 120 A nucleotides.

A RNA molecule may encompass:
(i) capped unmodified RNA molecule;
(ii) capped modified RNA molecule;
(iii) uncapped unmodified RNA molecule;
(iv) uncapped modified RNA molecule.

### Capped and Uncapped RNA Molecules

A “capped RNA molecule” refers to a RNA molecule of which 5′ end is linked to a guanosine or a modified guanosine, for example a 7-methylguanosine (m7G), connected to a 5′ to 5′ triphosphate linkage or analog. This definition is commensurate with the most widely-accepted definition of a 5′cap.

“Cap analogs” include caps which are biologically equivalent to a 7-methylguanosine (m7G), connected to a 5′ to 5′ triphosphate linkage, and which can thus be also substituted without impairing the protein expression of the corresponding messenger RNA in the eukaryotic host.

As example of caps, one may mention m7GpppN, m7GpppG, m7GppspG, m7GppspspG, m7GppspspG, m7Gppppm7G, m27′,3′—OGpppG, m27′,2′-OGpppG, m27′,2′—OGppspsG, or m27′,2′—OGpppspsG.

Examples of cap analogs can be: glyceryl, inverted deoxy abasic residue (moiety), 4′,5′ methylene nucleotide, 1-(beta-D-erythrofuranosyl)nucleotide, 4′-thio nucleotide, carbocyclic nucleotide, 1,5-anhydrohexitol nucleotide, L-nucleotides, alpha-nucleotide, modified base nucleotide, threo-pentofuranos I nucleotide, acyclic 3′,4′-seco nucleotide, acyclic 3,4-dihydroxybutyl nucleotide, acyclic 3,5 dihydroxypentyl nucleotide, 3′-3′-inverted nucleotide moiety, 3′-3′-inverted abasic moiety, 3′-2′-inverted nucleotide moiety, 3′-2′-inverted abasic moiety, 1,4-butanediol phosphate, 3′-phosphoramidate, hexylphosphate, aminohexyl phosphate, 3′-phosphate, 3′phosphorothioate, phosphorodithioate, or bridging or non-bridging methylphosphonate moiety.

Other examples of cap analogs include Anti-Reverse Cap Analogs (ARCAs), N1-methyl-guanosine, 2′-fluoro-guanosine, 7-deaza-guanosine, 8-oxo-guanosine, 2-amino-guanosine, LNA-guanosine, and 2-azido-guanosine.

Of note, among cap analogs, some are suitable for protein expression, but others may on the contrary hinder protein expression. Such distinction is understood by the man skilled in the art.

Providing a RNA with a 5′-cap or 5′-cap analog may be achieved by in vitro transcription of a DNA template in the presence of said 5′-cap or 5′-cap analog, wherein said 5′-cap is co-transcriptionally incorporated into the generated RNA strand, or the RNA may be generated, for example, by in vitro transcription, and 5′-cap may be attached to the RNA post-transcriptionally using capping enzymes, for example, capping enzymes of vaccinia virus.

An “uncapped RNA molecule” refers to any RNA molecule that does not belong to the definition of a “capped RNA molecule”.

Thus, according to a general embodiment, an “uncapped mRNA” may refer to a mRNA of which 5′ end is not linked to a 7-methylguanosine, through a 5′ to 5′ triphosphate linkage, or an analog as previously defined.

An uncapped RNA molecule, such as a messenger RNA, may be an uncapped RNA molecule having a (5′) ppp (5′), a (5′) pp (5′), a (5′) p (5′) or even a (5′) OH extremity. Such RNA molecules may be respectively abbreviated as 5′pppRNA; 5′ppRNA; 5′pRNA; 5′OHRNA.

In a non-limitative manner, the first base of an uncapped RNA molecule may be either an adenosine, a guanosine, a cytosine, or an uridine.

A RNA may not have uncapped 5′-triphosphates. Removal of such uncapped 5′-triphosphates can be achieved by treating RNA with a phosphatase.

### Modified and Unmodified RNA Molecules

A RNA may comprise further modifications, such as an extension or a truncation of the naturally occurring poly (A) tail or an alteration of 5′- or 3′-untranslated regions (UTR) such as introduction of an UTR which is not related to the coding region of the RNA, for example, the exchange of the existing 3′-UTR with or the insertion of at least one, for example two copies of a 3′-UTR derived from a globin gene, such as alpha 2-globin, alpha 1-globin, beta-globin, for example beta-globin, and for example human beta-globin.

A “modified RNA molecule” refers to an RNA molecule which contains at least one modified nucleotide, nucleoside sugar, or base, such as a modified purine or a modified pyrimidine. A modified nucleoside or base can be any nucleoside or base that is not A, U, C or G (respectively Adenosine, Uridine, Cytidine or Guanosine for nucleosides; and Adenine, Uracil, Cytosine or Guanine when referring solely to the sugar moiety).

An “unmodified RNA molecule” refers to any RNA molecule that is not commensurate with the definition of a modified RNA molecule.

The terms “modified and unmodified” are considered distinctly from the terms “capped and uncapped”, as the latter specifically relates to the base at 5′-end of a RNA.

The presence of modified nucleotide may increase the stability and/or decrease cytotoxicity of the nucleic acid. The term stability of RNA relates to the half-life of RNA, that is the period of time which is needed to eliminate half of the activity, amount, or number of molecules. The half-life of an RNA may be indicative of its stability. The half-life of RNA may influence the duration of expression of the RNA. It can be expected that an RNA having a long half-life will be expressed for an extended time-period.

In a non-limitative manner, examples of modified nucleotides, nucleosides and bases are disclosed in WO 2015/024667A1. A modified RNA may contain modified nucleotides, nucleosides or bases, including backbone modifications, sugar modifications or base modifications. Modified bases and/or modified RNA molecules are known in the art and are, for instance, taught in Warren et al. (“Highly Efficient Reprogramming to Pluripotency and Directed Differentiation of Human Cells with Synthetic Modified mRNA”; Cell Stem Cell; 2010), the content of which is incorporated by reference.

Sugar modifications include chemical modifications of the sugar of the nucleotides. Sugar modifications may consist in replacement or modification of the 2′ hydroxy (OH) group, which can be modified or replaced with a number of different “oxy” or “deoxy” substituents.

Examples of “oxy”-2′ hydroxyl group modifications include, but are not limited to, alkoxy or aryloxy (—OR, e.g., R═H, alkyl, cycloalkyl, aryl, aralkyl, heteroaryl or sugar); polyethyleneglycols (PEG), —O(CH2CH2O)nCH2CH2OR; “locked” nucleic acids (LNA) in which the 2′ hydroxyl is connected, e.g., by a methylene bridge, to the 4' carbon of the same ribose sugar; and amino groups (—O-amino, wherein the amino group, e.g., NRR, can be alkylamino, dialkylamino, heterocyclyl, arylamino, diarylamino, heteroarylamino, or diheteroaryl amino, ethylene diamine, polyamino) or aminoalkoxy.

“Deoxy” modifications include hydrogen, amino (e.g., NH2; alkylamino, dialkylamino, heterocyclyl, arylamino, diaryl amino, heteroaryl amino, diheteroaryl amino, or amino acid); or the amino group can be attached to the sugar through a linker, wherein the linker comprises at least one of the atoms C, N, and O.

The sugar group can also contain at least one carbon that possess the opposite stereochemical configuration than that of the corresponding carbon in ribose. Thus, a modified RNA can include nucleotides containing, for instance, arabinose as the sugar.

Backbone modifications include modifications, in which phosphates of the backbone of the nucleotides are chemically modified. The phosphate groups of the backbone can be modified by replacing at least one of the oxygen atoms with a different substituent. Further, the modified nucleosides and nucleotides can include the full replacement of an unmodified phosphate moiety with a modified phosphate as described herein.

Examples of modified phosphate groups include, but are not limited to, phosphorothioate, phosphoroselenates, borano phosphates, borano phosphate esters, hydrogen phosphonates, phosphoroamidates, alkyl or aryl phosphonates and phosphotriesters. Phosphorodithioates have both non-linking oxygens replaced by sulfur. The phosphate linker can also be modified by the replacement of a linking oxygen with nitrogen (bridged phosphoroamidates), sulfur (bridged phosphorothioates) and carbon (bridged methylene-phosphonates).

Base modifications include chemical modifications of the base moiety of the nucleotides. In this context nucleotide analogues or modifications are for example selected from nucleotide analogues which are suitable for transcription and/or translation of the RNA molecule in an eukaryotic cell. The modified nucleosides and nucleotides can be modified in the nucleobase moiety. For example, the nucleosides and nucleotides can be chemically modified on the major groove face. The major groove chemical modifications can include an amino group, a thiol group, an alkyl group, or a halo group. A modified base may be a modified purine base or a modified pyrimidine base. Examples of modified purine bases include modified adenosine and/or modified guanosine, such as hypoxanthine; xanthine; 7-methylguanine; inosine; xanthosine and 7-methylguanosine. Modified pyrimidine bases include modified cytidine and/or modified uridine, such as 5,6-dihydrouracil; pseudouridine; 5-methylcytidine; 5-hydroxymethylcytidine; dihydrouridine and 5-methylcytidine.

For examples, nucleotide analogues/modifications may be selected from the following base modifications: 2-amino-6-chloropurineriboside-5′-triphosphate, 2-aminopurine-riboside-5′-triphosphate; 2-aminoadenosine-5′-triphosphate, 2′-amino-2′-deoxycytidine-triphosphate, 2-thiocytidine-5′-triphosphate, 2-thiouridine-5′-triphosphate, 2′-fluorothymidine-5′-triphosphate, 2′—O-methyl inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-bromo-2′-deoxycytidine-5′-triphosphate, 5-bromo-2′-deoxyuridine-5′-triphosphate, 5-iodocytidine-5′-triphosphate, 5-iodo-2′-deoxycytidine-5′-triphosphate, 5-iodouridine-5′-triphosphate, 5-iodo-2′-deoxyuridine-5′-triphosphate, 5-methylcytidine-5′-triphosphate, 5-methyluridine-5′-triphosphate, 5-propynyl-2′-deoxycytidine-5′-triphosphate, 5-propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, O6-methylguanosine-5′-triphosphate, pseudouridine-5′-triphosphate, or puromycin-5′-triphosphate, and xanthosine-5′-triphosphate.

Modified nucleosides may be selected from a list consisting of: pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, I-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine/4-methoxy-pseudouridine, and 4-methoxy-2-thio-pseudouridine.

Modified nucleosides and nucleotides may include 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

Modified nucleosides may include 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2, 6-diaminopurine, 7-deaza-8-aza-2, 6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine.

Modified nucleosides may include inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, I-methyl-6-thio-guanosine, N2-methyl-6-thio-guanosine, and N2,N2-dimethyl-6-thio-guanosine.

RNAs having an unmasked poly-A sequence may translated more efficiently than RNAs having a masked poly-A sequence. “Unmasked poly-A sequence” means that the poly-A sequence at 3′ end of an RNA molecule ends with an A of the poly-A sequence and is not followed by nucleotides other than A located at the 3′ end, i.e., downstream, of the poly-A sequence. Furthermore, a long poly-A sequence of about 120 base pairs results in an optimal transcript stability and translation efficiency of RNA.

Therefore, in-order-to increase stability and/or expression of an RNA, the poly-A sequence may be modified, for example, for having a length of 10 to 500, for example 30 to 300, for example 65 to 200 and for example 100 to 150 adenosine residues. A poly-A sequence may have a length of approximately 120 adenosine residues. To further increase stability and/or expression of an RNA, the poly-A sequence can be unmasked.

Incorporation of a 3′-non-translated region (UTR) into 3′-non-translated region of an RNA molecule can result in an enhancement in translation efficiency. A synergistic effect may be achieved by incorporating two or more of such 3′-non-translated regions. The 3′-non-translated regions may be autologous or heterologous to the RNA into which they are introduced. The 3′-non-translated region may be derived from the human β-globin gene.

A combination of the above-described modifications, i.e., incorporation of a poly-A sequence, unmasking of a poly-A sequence and incorporation of at least one 3′-non-translated region, may have a synergistic influence on the stability of RNA and increase in translation efficiency.

The expression of an RNA may be further increased by modification of the sequence encoding the peptide or protein, for example by increasing the GC-content to increase mRNA stability and/or by performing a codon optimization to enhance translation in cells.

### Pharmaceutical Compositions and Uses Thereof

The compositions or LNPs disclosed herein may be used in a pharmaceutical composition. A pharmaceutical composition may comprise a composition or LNPs disclosed herein and at least one pharmaceutically acceptable excipient.

A composition or LNPs may comprise a biologically active agent as disclosed herein.

The biologically active agent may be a nucleic acid as disclosed herein.

It is also disclosed immunogenic compositions comprising at least a composition or a LNP comprising a nucleic acid encoding at least an antigen.

In some embodiments, compositions or LNPs disclosed herein, containing at least one biologically active agent, for example a nucleic acid, may be for use as a medicament.

In some embodiments, compositions or LNPs disclosed herein, containing at least one biologically active agent, for example a nucleic acid, may be for use in a method for preventing and/or treating a disease selected in a group consisting of infectious diseases, allergies, autoimmune diseases, blood disorders, metabolic diseases, neurologic diseases, and cancer diseases.

Also is disclosed a method for manufacturing a medicament or a pharmaceutical composition, comprising at least the steps of mixing a composition or LNPs as disclosed here, comprising at least one biologically active agent, for example a nucleic acid, with at least one pharmaceutically acceptable excipient.

The method for manufacturing a medicament or a pharmaceutical composition may further comprise the steps of preparing a composition of the LNPs as above indicated.

The method may further comprise a step of suspending or diluting the composition or LNPs in a pharmaceutically acceptable solvent.

An “pharmaceutically acceptable solvent” may be any solvent suitable for resuspending or dissolving the freeze-dried LNPs and pharmaceutically accepted for an enteral or parenteral administration to an individual in need thereof. A pharmaceutically acceptable solvent may be water for injection or a buffer, such as saline, a citrate, a histidine, or a phosphate buffer.

A pharmaceutical or an immunogenic composition may be sterile.

General guidelines for the formulation and manufacture of pharmaceutical compositions and agents are available, for example, in Remington's The Science and Practice of Pharmacy, 21St Edition, A. R. Gennaro; Lippincott, Williams & Wilkins, Baltimore, Md., 2006. Any pharmaceutically acceptable excipients may be used in a pharmaceutical composition, except insofar as an excipient may be incompatible with one or more components of an LNP.

The compositions as disclosed herein may be formulated into preparations in solid, semi-solid, liquid forms, such as powders, solutions, suspensions, or injections.

Exemplary pharmaceutically acceptable excipients that may be used may be selected from diluents, such as water for injection, or physiological salt solutions, such as amino acids buffers (histidine, arginine, glycine, proline, glycylglycine), saline buffers (inorganic salts NaCl, calcium chloride), phosphate buffers, acetate buffers, citrate buffers, succinate buffers; sugars or polyalcohols such as dextrose, glycerol, ethanol, sucrose, trehalose, mannitol; surfactants such as Polysorbate 80, polysorbate 20, poloxamer 188; and the like, as well as combination thereof. In many cases, it will be preferable to include isotonic agents, such as sugars, polyalcohols, or sodium chloride in the composition, and formulation may also contain an anti-oxidant such as tryptamine and a stabilizing agent such as Tween 20 or 80, other solvents such as monohydric alcohols, such as ethanol, or isopropanol, and polyhydric alcohols such as glycols and edible oils such as soybean oil, coconut oil, olive oil, safflower oil, cottonseed oil, oily esters such as ethyl oleate, isopropyl myristate; binders, adjuvants, solubilizers, thickening agents, stabilizers, disintegrants, lubricating agents, buffering agents, emulsifiers, wetting agents, suspending agents, sweetening agents, colorants, flavors, preservatives, anti-oxidants, processing agents, drug delivery modifiers and enhancers such as calcium phosphate, magnesium stearate, talc, monosaccharides, disaccharides, starch, gelatin, cellulose, methylcellulose, sodium carboxymethyl cellulose, dextrose, hydroxypropyl-β-cyclodextrin, polyvinylpyrrolidone or polyethylene glycol. Pharmaceutically acceptable excipients may also include any and all solvents, dispersion media, coatings, anti-bacterial and anti-fungal agents, and the like that are physiologically compatible.

Appropriate concentrations and dosages can be readily determined by one skilled in the art.

Administration of pharmaceutical and immunogenic compositions as disclosed herein may be carried out via any of the accepted modes of administration of compositions for serving similar utilities.

Typical routes of administering such pharmaceutical and immunogenic compositions include, without limitation, oral, topical, transdermal, inhalation, parenteral, sublingual, buccal, intranasal. The term parenteral as used herein includes subcutaneous injections, intravenous, intramuscular, intradermal, intrasternal injection or infusion techniques.

The pharmaceutical and immunogenic compositions may be administered by any suitable route, depending on parameters known in the art, such as the form of the composition (solid or liquid), the individual to be treated, the nature of the therapeutic agent contained in the LNPs, etc.

For example, a pharmaceutical or an immunogenic composition may be administered systemically, orally, sublingually, intranasally, intradermally, or subcutaneously.

For parenteral administration in an aqueous solution, for example, the solution should be suitably buffered if necessary and the liquid diluent first rendered isotonic with sufficient saline or glucose. These aqueous solutions are especially suitable for intravenous, intramuscular, subcutaneous and intraperitoneal administration. In this connection, sterile aqueous media which can be employed will be known to those of skill in the art.

In some embodiments, a pharmaceutical or an immunogenic composition may be suitable for subcutaneous administration.

In some embodiments, a pharmaceutical or an immunogenic composition may be suitable for intramuscular administration.

Actual methods of preparing such dosage forms are known, or will be apparent, to those skilled in this art; for example, see Remington: The Science and Practice of Pharmacy, 20th Edition (Philadelphia College of Pharmacy and Science, 2000).

The compositions may contain at least one inert diluent or carrier.

In one embodiment, the composition may be in the form of a liquid, for example, a solution, an emulsion or a suspension. The liquid may be for delivery by injection. Compositions intended to be administered by injection may contain at least one of: a surfactant, preservative, wetting agent, dispersing agent, suspending agent, buffer, stabilizer and isotonic agent may be included. The liquid compositions as disclosed herein may include at least one of: sterile diluents such as water for injection, saline solution, for example physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose; agents to act as cryoprotectants such as sucrose or trehalose.

The parenteral preparation can be enclosed in ampoules, disposable syringes or multiple dose vials made of glass or plastic. An injectable pharmaceutical composition is for example sterile.

The pharmaceutical and immunogenic compositions as disclosed herein may be prepared by methodology well known in the pharmaceutical art. For example, a pharmaceutical composition intended to be administered by injection can be prepared by combining the lipid nanoparticles as disclosed herein with sterile, distilled water or other carrier so as to form a solution. A surfactant may be added to facilitate the formation of a homogeneous solution or suspension.

The compositions as disclosed herein are administered in a therapeutically effective amount, which will vary depending upon a variety of factors including the activity of the specific therapeutic agent employed; the metabolic stability and length of action of the therapeutic agent; the age, body weight, general health, sex, and diet of the patient; the mode and time of administration; the rate of excretion; the drug combination; the severity of the particular disorder or condition; and the subject undergoing therapy.

Compositions as disclosed herein may also be administered simultaneously with, prior to, or after administration of at least one other therapeutic agent. Such combination therapy includes administration of a single pharmaceutical dosage formulation of a composition as disclosed herein and at least one additional active agent, as well as administration of the composition as disclosed herein and each active agent in its own separate pharmaceutical dosage formulation. Where separate dosage formulations are used, the compositions as disclosed herein and at least one additional active agent can be administered at essentially the same time, i.e., concurrently, or at separately staggered times, i.e., sequentially; combination therapy is understood to include all these regimens.

A pharmaceutical or an immunogenic composition may be administrated through drug combination devices, such multi-chamber syringes, in which at least one chamber is containing the pharmaceutical composition in solid form and at least one chamber is containing a pharmaceutically acceptable solvent for suspending or dissolving the composition.

In some embodiments, LNPs as disclosed herein may comprising at least one nucleic acid encoding for an antigen from an Influenza A virus and/or an Influenza B virus. Such LNPs may be for use for preventing or treating an Influenza A and/or an Influenza B virus infection. Such LNPs may be for use as an immunogenic composition against an Influenza A virus and/or an Influenza B virus.

In some embodiments, the present disclosure relates to LNPs comprising a lipid component comprising a cationic ionizable lipid, a neutral lipid, a structural lipid, and optionally a PEG-lipid, and comprising at least one nucleic acid encoding for an antigen from an Influenza A virus and/or an Influenza B virus. Such LNPs may be for use for preventing or treating an Influenza A and/or an Influenza B virus infection. Such LNPs may be for use as an immunogenic composition against an Influenza A virus and/or an Influenza B virus.

In some embodiments, LNPs as disclosed herein may comprising at least one nucleic acid encoding for an antigen from a Respiratory syncytial A virus and/or a Respiratory syncytial B virus. Such LNPs may be for use for preventing or treating a Respiratory syncytial A virus and/or a Respiratory syncytial B virus infection. Such LNPs may be for use as an immunogenic composition against a Respiratory syncytial A virus and/or a Respiratory syncytial B virus.

In some embodiments, the present disclosure relates to LNPs comprising a lipid component comprising a cationic ionizable lipid, a neutral lipid, a structural lipid, and optionally a PEG-lipid, and comprising at least one nucleic acid encoding for an antigen from a Respiratory syncytial A virus and/or a Respiratory syncytial B virus. Such LNPs may be for use for preventing or treating a Respiratory syncytial A virus and/or a Respiratory syncytial B virus infection. Such LNPs may be for use as an immunogenic composition against a Respiratory syncytial A virus and/or a Respiratory syncytial B virus.

In some embodiments, LNPs as disclosed herein may comprising at least one nucleic acid encoding for a SARS-Cov2 antigen. Such LNPs may be for use for preventing or treating a SARS-Cov-2 infection. Such LNPs may be for use as an immunogenic composition against SARS-Cov-2.

In some embodiments, the present disclosure relates to LNPs comprising a lipid component comprising a cationic ionizable lipid, a neutral lipid, a structural lipid, and optionally a PEG-lipid, and comprising at least one nucleic acid encoding for a SARS-Cov2 antigen. Such LNPs may be for use for preventing or treating a SARS-Cov-2 infection. Such LNPs may be for use as an immunogenic composition against SARS-Cov-2.

In some embodiments, the compositions or the LNPs as disclosed herein and comprising at least one nucleic acid may be used in the manufacture of a medicament.

Some embodiments relate to a use of the compositions or the LNPs as disclosed herein and comprising at least one biologically active agent in the manufacture of a pharmaceutical composition.

### Methods of Treatment

In some embodiments, the disclosure also relates to a method of preventing and/or treating a disease in an individual in need thereof, wherein the method comprises administering an effective amount of a composition or LNPs as disclosed herein, comprising at least one biologically active agent, to said individual. For example, compositions or LNPs as disclosed herein may be for use in a therapeutic method for preventing and/or treating infectious diseases, allergies, autoimmune diseases, blood disorders, metabolic diseases, neurologic diseases, and tumour or cancer diseases.

In some embodiments, blood disorders, metabolic diseases, neurologic diseases may be rare diseases. A rare disease is a disease that affects a small percentage of the population, for example with an incidence ranging from about 1/1,000 to about 1/200,000 people.

For example, diseases which may be concerned by the disclosure may infectious diseases such as viral infectious diseases, bacterial infectious diseases, fungal or parasitic infectious diseases. Diseases also concerned by the disclosure may be cancer or tumour diseases.

Viral infectious diseases may be acute febrile pharyngitis, pharyngoconjunctival fever, epidemic keratoconjunctivitis, infantile gastroenteritis, Coxsackie infections, infectious mononucleosis, Burkitt lymphoma, acute hepatitis, chronic hepatitis, hepatic cirrhosis, hepatocellular carcinoma, primary HSV-1 infection (e.g., gingivostomatitis in children, tonsillitis and pharyngitis in adults, keratoconjunctivitis), latent HSV-1 infection (e.g., herpes labialis and cold sores), primary HSV-2 infection, latent HSV-2 infection, aseptic meningitis, infectious mononucleosis, Cytomegalic inclusion disease, Kaposi sarcoma, multicentric Castleman disease, primary effusion lymphoma, AIDS, influenza, Reye syndrome, measles, postinfectious encephalomyelitis, Mumps, hyperplastic epithelial lesions (e.g., common, flat, plantar and anogenital warts, laryngeal papillomas, epidermodysplasia verruciformis), cervical carcinoma, squamous cell carcinomas, croup, pneumonia, bronchiolitis, common cold, Poliomyelitis, Rabies, bronchiolitis, pneumonia, influenza-like syndrome, severe bronchiolitis with pneumonia, German measles, congenital rubella, Varicella, Covid-19, Respiratory Syncytial Virus (RSV) infection, and herpes zoster.

In one embodiment, the disease is influenza, a Respiratory Syncytial Virus (RSV) infection, or Covid-19, and for example is influenza.

Bacterial infectious diseases may be such as abscesses, actinomycosis, acute prostatitis, Aeromonas hydrophila, annual ryegrass toxicity, anthrax, bacillary peliosis, bacteremia, bacterial gastroenteritis, bacterial meningitis, bacterial pneumonia, bacterial vaginosis, bacterium-related conditions, cutaneous bartonellosis, BCG-oma, botryomycosis, botulism, Brazilian purpuric fever, Brodie abscess, brucellosis, Buruli ulcer, campylobacteriosis, caries, Carrion's disease, cat scratch disease, cellulitis, chlamydia infection, cholera, chronic bacterial prostatitis, chronic recurrent multifocal osteomyelitis, clostridial necrotizing enteritis, combined periodontic-endodontic lesions, contagious bovine pleuropneumonia, diphtheria, diphtheritic stomatitis, ehrlichiosis, erysipelas, piglottitis, erysipelas, Fitz-Hugh-Curtis syndrome, flea-borne spotted fever, foot rot (infectious pododermatitis), Garre's sclerosing osteomyelitis, Gonorrhea, Granuloma inguinale, human granulocytic anaplasmosis, human monocytotropic ehrlichiosis, hundred days' cough, impetigo, late congenital syphilitic oculopathy, legionellosis, Lemierre's syndrome, leprosy (Hansen's Disease), leptospirosis, listeriosis, Lyme disease, lymphadenitis, melioidosis, meningococcal disease, meningococcal septicaemia, methicillin-resistant Staphylococcus aureus (MRS A) infection, Mycobacterium avium-intracellulare (MAI), mycoplasma pneumonia, necrotizing fasciitis, nocardiosis, noma (cancrum oris or gangrenous stomatitis), omphalitis, orbital cellulitis, osteomyelitis, overwhelming post-splenectomy infection (OPSI), ovine brucellosis, pasteurellosis, periorbital cellulitis, pertussis (whooping cough), plague, pneumococcal pneumonia, Pott disease, proctitis, pseudomonas infection, psittacosis, pyaemia, pyomyositis, Q fever, relapsing fever (typhinia), rheumatic fever, Rocky Mountain spotted fever (RMSF), rickettsiosis, salmonellosis, scarlet fever, sepsis, serratia infection, shigellosis, southern tick-associated rash illness, staphylococcal scalded skin syndrome, streptococcal pharyngitis, swimming pool granuloma, swine brucellosis, syphilis, syphilitic aortitis, tetanus, toxic shock syndrome (TSS), trachoma, trench fever, tropical ulcer, tuberculosis, tularemia, typhoid fever, typhus, urogenital tuberculosis, urinary tract infections, vancomycin-resistant Staphylococcus aureus infection, Waterhouse-Friderichsen syndrome, pseudotuberculosis (Yersinia) disease, and yersiniosis.

Parasitic infectious diseases may be a giardiasis, trichomoniasis, African Sleeping Sickness, American Sleeping Sickness, leishmaniasis (Kala-Azar), balantidiasis, toxoplasmosis, malaria, acanthamoeba keratitis, and babesiosis.

Fungal infectious diseases may be aspergilloses, blastomycosis, candidasis, coccidioidomycosis, cryptococcosis, histoplasmosis, mycetomas, paracoccidioidomycosis, and tinea pedis. Furthermore, persons with immuno-deficiencies are for example susceptible to disease by fungal genera such as Aspergillus, Candida, Cryptoccocus, Histoplasma, and Pneumocystis. Other fungi can attack eyes, nails, hair, and especially skin, the so-called dermatophytic fungi and keratinophilic fungi, and cause a variety of conditions, of which ringworms such as athlete's foot are common. Fungal spores are also a major cause of allergies, and a wide range of fungi from different taxonomic groups can evoke allergic reactions in some people.

Cancer or tumour diseases may be cancer or tumor diseases are for example selected from melanomas, malignant melanomas, colon carcinomas, lymphomas, sarcomas, blastomas, renal carcinomas, gastrointestinal tumors, gliomas, prostate tumors, bladder cancer, rectal tumors, stomach cancer, oesophageal cancer, pancreatic cancer, liver cancer, mammary carcinomas (=breast cancer), uterine cancer, cervical cancer, acute myeloid leukaemia (AML), acute lymphoid leukaemia (ALL), chronic myeloid leukaemia (CML), chronic lymphocytic leukaemia (CLL), hepatomas, various virus-induced tumors such as, for example, papilloma virus-induced carcinomas (e.g. cervical carcinoma=cervical cancer), adenocarcinomas, herpes virus-induced tumors (e.g. Burkitt's lymphoma, EBV-induced B-cell lymphoma), heptatitis B-induced tumors (hepatocell carcinomas), HTLV-1- and HTLV-2-induced lymphomas, acoustic neuroma, lung carcinomas (=lung cancer=bronchial carcinoma), small-cell lung carcinomas, pharyngeal cancer, anal carcinoma, glioblastoma, rectal carcinoma, astrocytoma, brain tumors, retinoblastoma, basalioma, brain metastases, medulloblastomas, vaginal cancer, pancreatic cancer, testicular cancer, Hodgkin's syndrome, meningiomas, Schneeberger disease, hypophysis tumor, Mycosis fungoides, carcinoids, neurinoma, spinalioma, Burkitt's lymphoma, laryngeal cancer, renal cancer, thymoma, corpus carcinoma, bone cancer, non-Hodgkin's lymphomas, urethral cancer, CUP syndrome, head/neck tumors, oligodendroglioma, vulval cancer, intestinal cancer, colon carcinoma, oesophageal carcinoma (=oesophageal cancer), wart involvement, tumors of the small intestine, craniopharyngeomas, ovarian carcinoma, genital tumors, ovarian cancer (=ovarian carcinoma), pancreatic carcinoma (=pancreatic cancer), endometrial carcinoma, liver metastases, penile cancer, tongue cancer, gall bladder cancer, leukaemia, plasmocytoma, lid tumor, prostate cancer (=prostate tumors).

Diseases for which the present disclosure can be useful as a therapeutic intervention include diseases such as SMN1-related spinal muscular atrophy (SMA); amyotrophic lateral sclerosis (ALS); GALT-related galactosemia; Cystic Fibrosis (CF); SLC3A1-related disorders including cystinuria; COL4A5-related disorders including Alport syndrome; galactocerebrosidase deficiencies; X-linked adrenoleukodystrophy and adrenomyeloneuropathy; Friedreich's ataxia; Pelizaeus-Merzbacher disease; TSC1 and TSC2-related tuberous sclerosis; Sanfilippo B syndrome (MPS IIIB); CTNS-related cystinosis; the FMR1-related disorders which include Fragile X syndrome, Fragile X-Associated Tremor/Ataxia Syndrome and Fragile X Premature Ovarian Failure Syndrome; Prader-Willi syndrome; hereditary hemorrhagic telangiectasia (AT); Niemann-Pick disease Type C₁; the neuronal ceroid lipofuscinoses-related diseases including Juvenile Neuronal Ceroid Lipofuscinosis (JNCL), Juvenile Batten disease, Santavuori-Haltia disease, Jansky-Bielschowsky disease, and PTT-1 and TPP1 deficiencies; EIF2B1, EIF2B2, EIF2B3, EIF2B4 and EIF2B5-related childhood ataxia with central nervous system hypomyelination/vanishing white matter; CACNA1A and CACNB4-related Episodic Ataxia Type 2; the MECP2-related disorders including Classic Rett Syndrome, MECP2-related Severe Neonatal Encephalopathy and PPM-X Syndrome; CDKL5-related Atypical Rett Syndrome; Kennedy's disease (SBMA); Notch-3 related cerebral autosomal dominant arteriopathy with subcortical infarcts and leukoencephalopathy (CADASIL); SCN1A and SCN1B-related seizure disorders; the Polymerase G-related disorders which include Alpers-Huttenlocher syndrome, POLG-related sensory ataxic neuropathy, dysarthria, and ophthalmoparesis, and autosomal dominant and recessive progressive external ophthalmoplegia with mitochondrial DNA deletions; X-Linked adrenal hypoplasia; X-linked agammaglobulinemia; Fabry disease; and Wilson's disease.

In one embodiment, the nucleic acids, and for example mRNA, of the present disclosure may encode functional proteins or enzymes. For example, the compositions of the present disclosure may include mRNA encoding erythropoietin (EPO), a1-antitrypsin, carboxypeptidase N, alpha galactosidase (GLA), ornithine carbamoyltransferase (OTC), or human growth hormone (hGH).

In other embodiments, the disclosure relates to methods of transfecting at least one isolated target cell with a nucleic acid, wherein said method comprises contacting the at least one target cell with an effective amount of at least one nucleic acid polynucleotide and (i) at least one nucleic acid and at least one lipidic compound as disclosed herein, or (ii) at least one composition as described herein containing a nucleic acid, or (iii) at least one lipid nanoparticle containing a nucleic acid as described herein, such that the at least one target cell are transfected with said nucleic acid.

Target cells include, but are not limited to lymph nodes, hepatocytes, epithelial cells, hematopoietic cells, epithelial cells, endothelial cells, lung cells, bone cells, stem cells, mesenchymal cells, neural cells (e.g., meninges, astrocytes, motor neurons, cells of the dorsal root ganglia and anterior horn motor neurons), photoreceptor cells (e.g., rods and cones), retinal pigmented epithelial cells, secretory cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, antigen presenting cells such as dendritic cells, reticulocytes, leukocytes, granulocytes and tumor cells.

In one embodiment, the cells targeted may be spleen, liver, lung, heart and kidney cells. In another embodiment, the cells targeted may be spleen and kidney cells, and for example may be spleen cells.

In some embodiments, lipid nanoparticles or compositions as disclosed herein which allow avoiding hepatic clearance may be of particular interest.

Following transfection of at least one target cell by, for example, the nucleic acid encapsulated in the lipid nanoparticles, the production of a polypeptide or a protein encoded by such nucleic acid may be for example stimulated and the capability of such target cells to express the nucleic acid and produce, for example, a polypeptide or protein of interest is enhanced. For example, transfection of a target cell by a composition encapsulating mRNA will enhance (i.e., increase) the production of the protein or enzyme encoded by such mRNA.

In other embodiments, the disclosure relates to methods of producing a polypeptide in at least one target cell, wherein said method comprises contacting the at least one target cell with an effective amount of (i) at least one nucleic acid and at least one lipidic compound as disclosed herein, or (ii) at least one composition as herein described containing a nucleic acid, or (iii) at least one lipid nanoparticle containing a nucleic acid as described herein, such that the at least one target cell are transfected with the nucleic acid operably encoding said polypeptide.

It is to be understood that the disclosure encompasses all variations, combinations, and permutations in which at least one limitation, element, clause, descriptive term, etc., from at least one of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, e.g., in Markush group or similar format, it is to be understood that each subgroup of the elements is also disclosed, and any element(s) can be removed from the group. It should be understood that, in general, where the disclosure, or aspects of the disclosure, is/are referred to as comprising particular elements, features, etc., they also encompass embodiments consisting, or consisting essentially of, such elements, features, etc. For purposes of simplicity those embodiments have not in every case been specifically set forth in so many words herein. It should also be understood that any embodiment or aspect of the disclosure can be explicitly excluded from the claims, regardless of whether the specific exclusion is recited in the specification. The publications and other reference materials referenced herein to describe the background of the disclosure and to provide additional detail regarding its practice are hereby incorporated by reference.

The following examples are provided for purpose of illustration and not limitation.

### EXAMPLES

### Materials and Methods

### Nuclear Magnetic Resonance Spectroscopy (H, C NMR)

-H and C NMR spectra were recorded at room temperature on the following spectrometer: Brucker Advance 400 (NMR H: 400 MHz and NMR C: 75 MHZ).

Recorded shifts were reported in parts per million (δ) and calibrated using residual undeuterated 3: H 7.26 ppm; C 77.16 ppm, MeOH H 3.31 ppm; C: 49.0 ppm). Data were represented as follows, chemical shift, multiplicity (s=singlet, d=doublet, t=triplet, q=quartet and m=multiplet), coupling constant (J in Hz), integration and attribution.

NMR spectra were obtained using the commercial software NMRnotebook.

-High-resolution mass spectra (HRMS) were obtained using an Agilent Q-TOF (time of flight) 6520 and Low-resolution mass spectra (LCMS) using an Agilent MSD 1200 SL (ESI/APCl) with an Agilent HPLC 1200 SL.

### Example 1: Synthesis of 2-[2-[2-[2-[2,3-bis (8-nonoxy-8-oxo-1-methylpiperidine-4-carboxylate octoxy) propoxy] ethoxy]ethoxy] ethoxy]ethyl (compound VI)

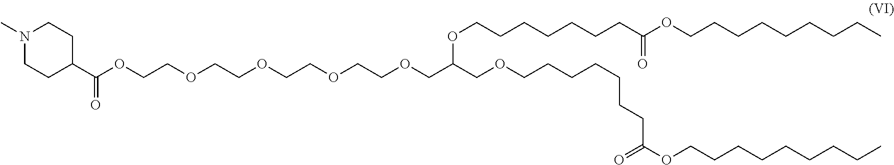

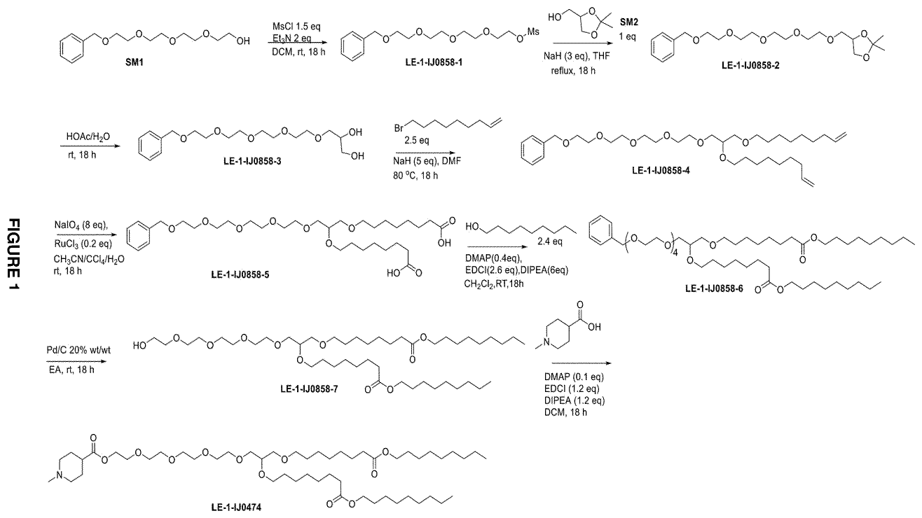

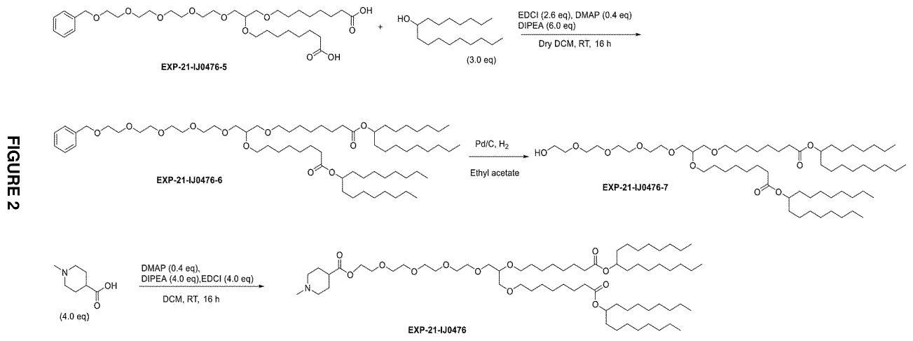

The compound VI is prepared according to the schema of synthesis submitted on FIG. 1.

Synthesis of the intermediate LE-1-IJ0858-1

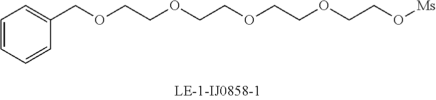 [SMILES: CS(=O)(=O)OCCOCCOCCOCCOCc1ccccc1]

2-[2-[2-(2-benzyloxyethoxy) ethoxy]ethoxy] ethanol (50 g, 176 mmol) and triethylamine (35.6 g, 352 mmol) in dry dichloromethane (500 mL) under nitrogen were cooled to −5° C. Methanesulfonyl chloride (30.2 g, 264 mmol) in dry DCM (20 mL) was added dropwise to this solution at 0° C. The mixture was allowed to warm to room temperature and stirred at room temperature for 18 h. Triethylamine hydrochloride was filtered off, and the DCM solution was washed with 0.1 N HCl and dried over sodium sulfate. Removing the solvent afforded 2-[2-[2-(2-benzyloxyethoxy) ethoxy]ethoxy]ethyl methanesulfonate (69.1 g, 175 mmol, quant.) as light yellow oil which was used without further purification.

1H NMR (400 MHZ, CDCl3) δ 7.37-7.27 (m, 5H), 4.56 (s, 2H), 4.39-4.33 (m, 2H), 3.78-3.73 (m, 2H), 3.69-3.60 (m, 12H), 3.06 (s, 3H).

### Synthesis of the intermediate LE-1-IJ0858-2

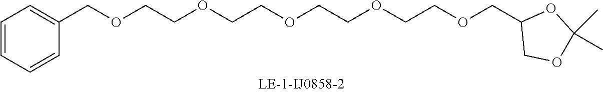 [SMILES: CC1(C)OCC(COCCOCCOCCOCCOCc2ccccc2)O1]

To the solution of (2,2-dimethyl-1,3-dioxolan-4-yl) methanol (24.4 g, 175 mmol) in THF (500 mL) was added NaH (14 g, 351 mmol) and the mixture was heated to reflux for 15 min. Then the reaction was cooled to room temperature and 2-[2-[2-(2-benzyloxyethoxy) ethoxy]ethoxy]ethyl methanesulfonate (69.1 g, 175 mmol) was added under nitrogen and the reaction was heated at 80° C. for 24 h. TLC indicated that the starting material was consumed. The reaction was quenched with water and extracted with ethyl acetate. The aqueous layer was extracted with ethyl acetate again. The combined organic layers were washed with brine, dried over sodium sulfate, filtered and concentrated. The residue was purified through flash chromatography eluted with 20 to 50% ethyl acetate in petroleum ether to give 24-[2-[2-[2-(2-benzyloxyethoxy) ethoxy]ethoxy] ethoxymethyl]-2,2-dimethyl-1,3-dioxolane (54.4 g, 70% yield) as light yellow oil.

1H NMR (400 MHZ, CDCl3) δ 7.38-7.27 (m, 5H), 4.57 (s, 2H), 4.28 (t, J=5.9 Hz, 1H), 4.05 (dd, J=8.3, 6.4 Hz, 1H), 3.72 (dd, J=8.3, 6.4 Hz, 1H), 3.70-3.61 (m, 16H), 3.57 (dd, J=10.0, 5.8 Hz, 1H), 3.49 (dd, J=10.0, 5.5 Hz, 1H), 1.42 (s, 3H), 1.35 (s, 3H).

### Synthesis of the intermediate LE-1-IJ0858-3

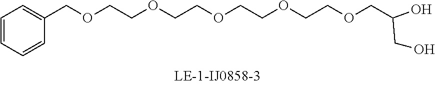 [SMILES: OCC(O)COCCOCCOCCOCCOCc1ccccc1]

The mixture of 4-[2-[2-[2-(2-benzyloxyethoxy) ethoxy]ethoxy] ethoxymethyl]-2,2-dimethyl-1,3-dioxolane (54.4 g, 123 mmol) in AcOH (200 mL) and H2O (200 mL) was stirred at room temperature for 18 h. TLC (EA/PE 1/1, SM Rf: 0.5; product, Rf: 0.1) indicated that all the starting materials was consumed. The solvent was removed under vacuum and azeotroped with toluene several times. 2-[2-[2-(2-methylsulfonyloxyethoxy) ethoxy]ethoxy]ethyl methanesulfonate (49 g, 123 mmol, quant.) as light yellow oil was obtained which was used without further purification.

1H NMR (400 MHZ, CDCl3) δ 7.38-7.27 (m, 5H), 4.57 (s, 2H), 3.88-3.81 (m, 1H), 3.70-3.51 (m, 21H).

### Synthesis of the intermediate LE-1-IJ0858-4

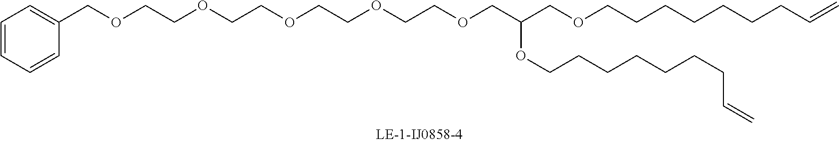 [SMILES: C=CCCCCCCCOCC(COCCOCCOCCOCCOCc1ccccc1)OCCCCCCCC=C]

To a solution of 3-[2-[2-[2-(2-benzyloxyethoxy) ethoxy]ethoxy] ethoxy]propane-1,2-diol (24 g, 60.3 mmol) in dry DMF (200 mL) under nitrogen was added NaH (9.64 g, 241 mmol) and the mixture was heated at 80° C. for 15 min. Then the reaction was cooled to room temperature and 9-bromonon-1-ene (31.9 g, 151 mmol) was added dropwise to this solution. The mixture was stirred at room temperature for 30 min and then at 80° C. for 18 h. TLC (EA/PE=1/1, Rf: 0.5) indicated that a new spot was formed. The reaction was quenched with water (50 mL) and then partitioned between ethyl acetate and water. The aqueous layer was extracted with ethyl acetate again. The combined organic layers were dried over sodium sulfate, filtered, and concentrated. The residue was purified by flash chromatography eluted with 20% to 50% ethyl acetate in petroleum ether to give 2-[2-[2-[2-[2,3-bis (non-8-enoxy) propoxy] ethoxylethoxylethoxy]ethoxymethylbenzene (9.3 g, 14.6 mmol, 24.2% yield) as light yellow oil.

1H NMR (400 MHZ, CDCl3) δ 7.37-7.27 (m, 5H), 5.89-5.72 (m, 2H), 5.04-4.89 (m, 4H), 4.57 (s, 2H), 3.71-3.60 (m, 17H), 3.59-3.38 (m, 9H), 2.03 (q, J=6.7 Hz, 4H), 1.60-1.49 (m, 4H), 1.43-1.23 (m, 16H).

### Synthesis of the intermediate LE-1-IJ0858-5

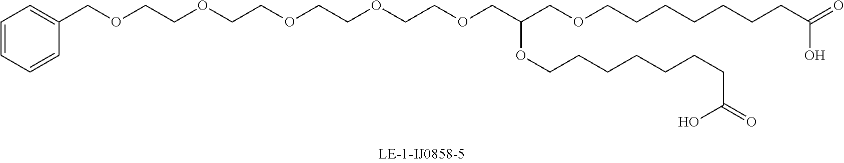 [SMILES: O=C(O)CCCCCCCOCC(COCCOCCOCCOCCOCc1ccccc1)OCCCCCCCC(=O)O]

To a solution of 2-[2-[2-[2-[2,3-bis (non-8-enoxy) propoxy] ethoxy]ethoxy] ethoxy]ethoxymethylbenzene (9.3 g, 14.6 mmol) in MeCN (80 mL), CCl4 (80 mL) and water (80 mL) was added NalO4 (24.9 g, 116 mmol) and RuCl3 (656 mg, 2.91 mmol). The reaction mixture was stirred at room temperature for 24 h. LCMS indicated that the title compound was the major product along with partial mono-aldehyde product. The reaction was filtered, and the filtrate was diluted with ethyl acetate (800 mL) and washed with 1N aq. HCl (400 mL). The organic layer was washed with Na2S2O3 solution and then dried over sodium sulfate, filtered and concentrated to give 8-[3-[2-[2-[2-(2-benzyloxyethoxy) ethoxy]ethoxy] ethoxy]-2-(7-carboxyheptoxy) propoxy] octanoic acid (10 g, 12.4 mmol) as yellow oil which was used without further purification.

8-[3-[2-[2-[2-(2-benzyloxyethoxy) ethoxy]ethoxy] ethoxy]-2-(8-oxooctoxy) propoxy] octanoic acid (10 g, 8 mmol) was dissolved in t-BuOH: H2O (3:1, 160 mL), containing NaH2PO4.2H2O (3.73 g, 24 mmol), 2-methy-2-butene (40 mL) and sodium chlorite (2.71 mg, 24 mmol). The reaction was stirred for 2 h at room temperature and LCMS indicated that the starting material was consumed. The reaction mixture was diluted with ethyl acetate. The aqueous layer was extracted with ethyl acetate. The combined organic layers were dried over sodium sulfate and concentrated to afford 8-[3-[2-[2-[2-(2-benzyloxyethoxy) ethoxy]ethoxy] ethoxy]-2-(7-carboxyheptoxy) propoxy] octanoic acid (10 g, 3.22 mmol, quant.) as light yellow oil.

1H NMR (400 MHZ, CDCl3) δ 7.38-7.27 (m, 5H), 4.57 (s, 2H), 3.71-3.61 (m, 17H), 3.59-3.37 (m, 9H), 2.33 (t, J=7.3 Hz, 4H), 1.69-1.51 (m, 8H), 1.39-1.28 (m, 14H).

### Synthesis of the intermediate LE-1-IJ0858-6

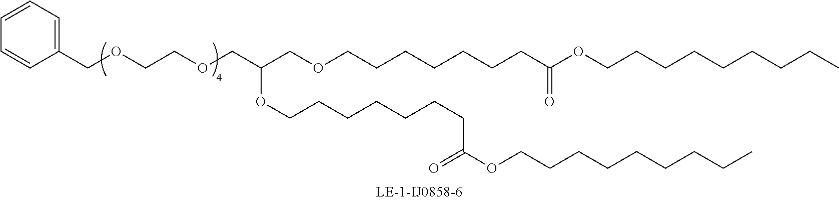

To the solution of 8-[3-[2-[2-[2-(2-benzyloxyethoxy) ethoxy]ethoxy] ethoxy]-2-(7-carboxyheptoxy) propoxy] octanoic acid (10 g, 14.8 mmol) and 1-Nonanol (5.12 g, 35.5 mmol) in dry dichloromethane (200 mL) under nitrogen were added N,N-Diisopropylethylamine (11.5 g, 88.7 mmol), 4-Dimethylaminopyridine (DMAP) (0.722 g, 5.91 mmol) and 1-Ethyl-3-(3-dimethylaminopropyl) carbodiimide (EDCl) (7.37 g, 38.4 mmol). The mixture was stirred at room temperature for 18 h. The reaction was diluted with dichloromethane and washed with brine. The organic layer was dried over sodium sulfate, filtered and concentrated. The residue was purified by flash chromatography eluted with 20% to 55% ethyl acetate in petroleum ether to give nonyl 8-[3-[2-[2-[2-(2-benzyloxyethoxy) ethoxy]ethoxy] ethoxy]-2-(8-nonoxy-8-oxo-octoxy) propoxy] octanoate (5 g, 35.9%) as colorless oil.

1H NMR (400 MHZ, CDCl3) δ 7.38-7.27 (m, 5H), 4.57 (s, 2H), 4.05 (t, J=6.8 Hz, 4H), 3.70-3.61 (m, 16H), 3.59-3.39 (m, 9H), 2.28 (t, J=7.5 Hz, 4H), 1.67-1.50 (m, 12H), 1.37-1.21 (m, 36H), 0.88 (t, J=6.8 Hz, 6H).

### Synthesis of the intermediate LE-1-IJ0858-7

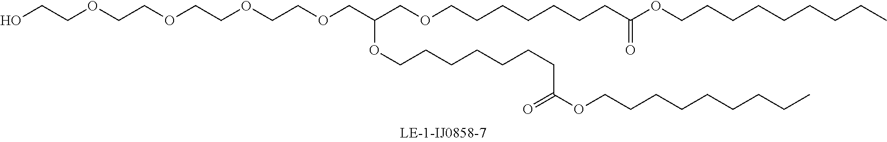 [SMILES: CCCCCCCCCOC(=O)CCCCCCCOCC(COCCOCCOCCOCCO)OCCCCCCCC(=O)OCCCCCCCCC]

To the solution of nonyl 8-[3-[2-[2-[2-(2-benzyloxyethoxy) ethoxy]ethoxy] ethoxy]-2-(8-nonoxy-8-oxo-octoxy) propoxy] octanoate (5 g, 5.31 mmol) in ethyl acetate (100 mL) was added Pd/C (1.13 g, 20% wt/wt). The mixture was stirred at room temperature under hydrogen for 18 h. TLC (ethyl acetate/petroleum ether 1/1) indicated that the starting material was consumed. The reaction was filtered through celite and washed with ethyl acetate to give nonyl 8-[3-[2-[2-[2-(2-hydroxyethoxy) ethoxy]ethoxy] ethoxy]-2-(8-nonoxy-8-oxo-octoxy) propoxy] octanoate (4.22 g, 4.98 mmol, 93.8%) as colorless oil.

1H NMR (400 MHZ, CDCl3) δ 4.05 (t, J=6.8 Hz, 4H), 3.74-3.38 (m, 27H), 2.28 (t, J=7.5 Hz, 4H), 1.68-1.50 (m, 12H), 1.39-1.21 (m, 37H), 0.88 (t, J=6.8 Hz, 6H).

### Synthesis of the Compound VI

To a solution of 1-methylpiperidine-4-carboxylic acid (0.152 g, 1.06 mol) and nonyl 8-[3-[2-[2-[2-(2-hydroxyethoxy) ethoxy]ethoxy] ethoxy]-2-(8-nonoxy-8-oxo-octoxy) propoxy] octanoate (0.6 g, 0.708 mol) in dichloromethane (15 ml) was added N,N-diisopropylethylamine (0.11 g, 0.85 mmol) and DMAP (8.65 mg, 0.07 mol) followed by N-(3-dimethylaminopropyl)-N′-ethylcarbodiimide hydrochloride (0.163 g, 0.85 mmol) portionwise. The reaction was allowed to stir at room temperature for 18 h. The reaction was diluted with dichloromethane and washed with saturated sodium bicarbonate. The organic layer was separated and washed with brine and dried over Na2SO4. The organic layer was filtered and evaporated under vacuum. The residue was purified by silica gel chromatography (0-15% methanol in dichloromethane) to 2-[2-[2-[2-[2,3-bis (8-nonoxy-8-oxo-octoxy) propoxy] ethoxy]ethoxy] ethoxy]ethyl 1-methylpiperidine-4-carboxylate (0.281 g, 0.29 mmol, 41% yield) as colorless oil.

1H NMR (400 MHZ, CDCl3) δ 4.27-4.20 (m, 2H), 4.05 (t, J=6.8 Hz, 4H), 3.72-3.61 (m, 14H), 3.59-3.39 (m, 9H), 2.84 (d, J=11.5 Hz, 2H), 2.38-2.23 (m, 8H), 2.06 (s, 1H), 1.99-1.87 (m, 3H), 1.86-1.77 (m, 2H), 1.67-1.52 (m, 12H), 1.37-1.23 (m, 36H), 0.88 (t, J=6.8 Hz, 6H).

MS (ESI) m/z=930.8 (M+H)+

### Example 2: Synthesis of 2-[2-[2-[2-[2,3-bis [8-(1-octylnonoxy)-8-oxo-octoxy] propoxy] ethoxy]ethoxy] ethoxy]ethyl 1-methylpiperidine-4-carboxylate (compound VII)

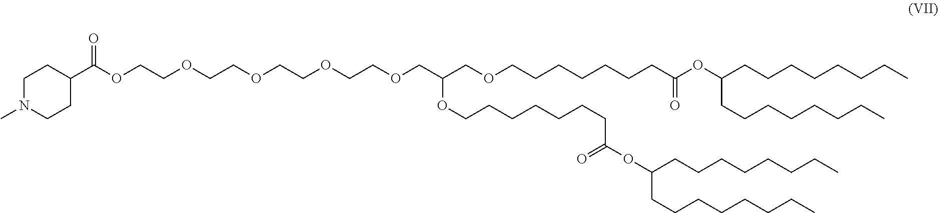 [SMILES: CCCCCCCCC(CCCCCCCC)OC(=O)CCCCCCCOCC(COCCOCCOCCOCCOC(=O)C1CCN(C)CC1)OCCCCCCCC(=O)OC(CCCCCCCC)CCCCCCCC]

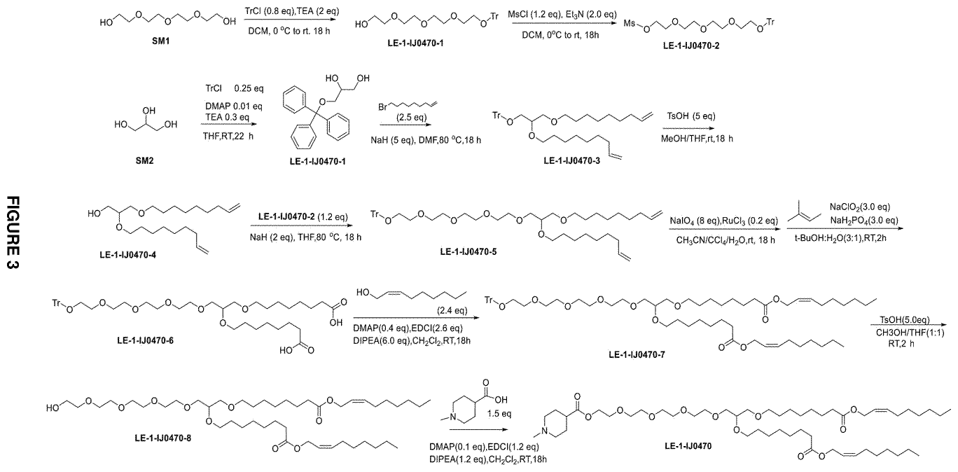

The compound VII is prepared according to the schema of synthesis submitted in FIG. 2.

### Synthesis of the intermediate EXP-21-IJ0476-6

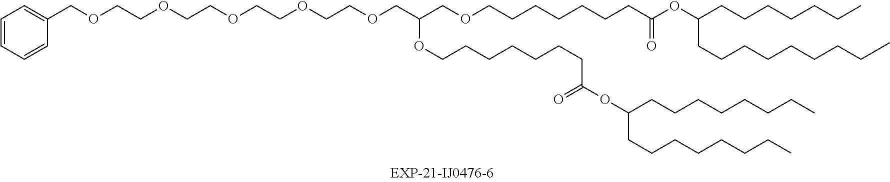 [SMILES: CCCCCCCCCC(CCCCCCC)OC(=O)CCCCCCCOCC(COCCOCCOCCOCCOCc1ccccc1)OCCCCCCCC(=O)OC(CCCCCCCC)CCCCCCCC]

To the solution of 8-[3-[2-[2-[2-(2-benzyloxyethoxy) ethoxy]ethoxy] ethoxy]-2-(7-carboxyheptoxy) propoxy] octanoic acid (1.26 g, 1.96 mmol) and heptadecan-8-ol (1.51 g, 5.88 mmol) in dry dichloromethane (20 mL) were added DIPEA (1.52 g, 11.8 mmol), DMAP (0.096 g, 0.784 mmol) and EDCl (0.977 g, 5.10 mmol) portionwise under ice bath.

The mixture was stirred at room temperature for 18 h. The reaction was quenched with NaHCO₃ (30 mL) and washed with brine. The organic layer was dried over sodium sulfate, filtered, and concentrated. The residue was purified by flash chromatography eluted with 0% to 5% (2%) CH₃OH in DCM to give 1-heptyldecyl 8-[3-[2-[2-[2-(2-benzyloxyethoxy) ethoxy]ethoxy] ethoxy]-2-[8-(1-octylnonoxy)-8-oxo-octoxy] propoxy] octanoate (0.600 g, 0.482 mmol, 24.6% yield) as colorless oil.

1H NMR (500 MHZ, CDCl3) δ 8.08-8.04 (m, 1H), 7.56 (t, J=7.4 Hz, 1H), 7.48-7.43 (m, 1H), 7.36-7.26 (m, 5H), 4.90-4.82 (m, 2H), 4.57 (s, 2H), 4.50-4.47 (m, 1H), 3.86-3.82 (m, 1H), 3.73-3.38 (m, 24H), 2.30-2.24 (m, 4H), 1.62-1.46 (m, 16H), 1.35-1.22 (m, 60H), 0.88 (t, J=6.9 Hz, 12H).

### Synthesis of the intermediate EXP-21-IJ0476-7

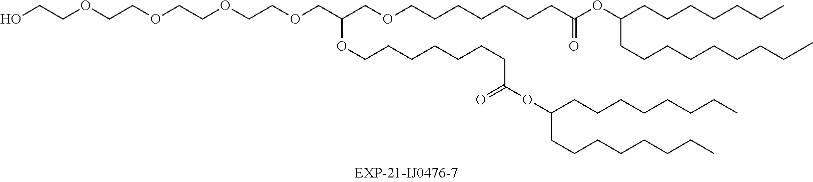 [SMILES: CCCCCCCCCC(CCCCCCC)OC(=O)CCCCCCCOCC(COCCOCCOCCOCCO)OCCCCCCCC(=O)OC(CCCCCCCC)CCCCCCCC]

A solution of 1-heptyldecyl 8-[3-[2-[2-[2-(2-benzyloxyethoxy) ethoxy]ethoxy] ethoxy]-2-[8-(1-octylnonoxy)-8-oxo-octoxy] propoxyloctanoate (0.600 g, 0.536 mmol) in EtOAc (10 mL) was purged for 10 minutes with N2 followed by addition of Pd/C (180 mg) and the reaction continued purging with N2. The reaction was next evacuated under vacuum and backfilled with H2 3 times. The reaction was next stirred overnight at room temperature under an atmosphere of H2. TLC (4% CH3OH in DCM) indicated that the reaction was finished. The slurry was filtered through celite and the celite was rinsed with EtOAc several times. The combined organics were next concentrated under vacuum to give 1-heptyldecyl 8-[3-[2-[2-[2-(2-hydroxyethoxy) ethoxy]ethoxy] ethoxy]-2-[8-(1-octylnonoxy)-8-oxo-octoxy] propoxy] octanoate (0.537 g, 0.495 mmol, 92.5% yield) as colorless oil.

1H NMR (500 MHZ, CDCl3) δ 4.85 (dd, J=12.5, 6.2 Hz, 2H), 3.79-3.32 (m, 25H), 2.32-2.23 (m, 4H), 1.64-1.46 (m, 16H), 1.35-1.20 (m, 60H), 0.88 (t, J=6.9 Hz, 12H).

Synthesis of the compound VII

To a solution of 1-heptyldecyl 8-[3-[2-[2-[2-(2-hydroxyethoxy) ethoxy]ethoxy] ethoxy]-2-[8-(1-octyl nonoxy)-8-oxo-octoxy] propoxy] octanoate (0.537 g, 0.522 mmol) and 1-methylpiperidine-4-carboxylic acid (0.299 g, 2.09 mmol) in dichloromethane (20 mL) was added DIPEA (0.270 g, 2.09 mmol) and DMAP (0.026 g, 0.209 mmol) followed by EDCl (0.400 g, 2.09 mmol) at 00C portionwise. The reaction was stirred at room temperature for 32 h. The reaction was diluted with dichloromethane and washed with saturated sodium bicarbonate. The organic layer was separated and washed with brine and dried over Na2SO4. The organic layer was filtered and evaporated under vacuum. The residue was purified by silica gel chromatography (0-10% CH3OH in DCM (4%)) to obtain 2-[2-[2-[2-[2,3-bis [8-(1-octylnonoxy)-8-oxo-octoxy] propoxy] ethoxy]ethoxy] ethoxy]ethyl 1-methylpiperidine-4-carboxylate (0.370 g, 0.308 mmol, 59.0% yield) as orange oil.

LCMS. MS (ESI) m/z=1155.9 (M+H)+

1H NMR (500 MHZ, CDCl3) δ 4.92-4.79 (m, 2H), 4.29-4.19 (m, 2H), 3.72-3.67 (m, 2H), 3.64 (d, J=6.4 Hz, 12H), 3.59-3.39 (m, 9H), 2.85 (s, 2H), 2.39-2.22 (m, 8H), 1.89 (d, J=56.9 Hz, 6H), 1.60-1.47 (m, 14H), 1.34-1.21 (m, 62H), 0.91-0.84 (m, 12H).

### Example 3: Synthesis of 2-[2-[2-[2-[2-[8-[(Z)-non-2-enoxy]-8-oxo-octoxy]-3-[8-[(Z)-oct-2-enoxy]-8-oxooctoxy] propoxy] ethoxy]ethoxy] ethoxy]ethyl 1-methylpiperidine-4-carboxylate (compound VIII)

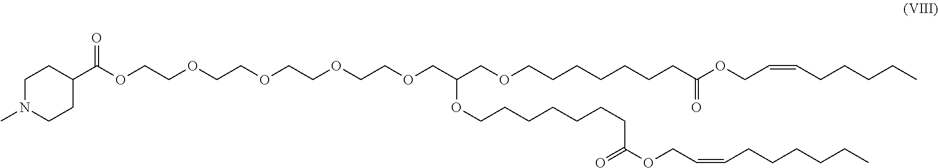 [SMILES: CCCCC/C=C\COC(=O)CCCCCCCOCC(COCCOCCOCCOCCOC(=O)C1CCN(C)CC1)OCCCCCCCC(=O)OC/C=C\CCCCCC]

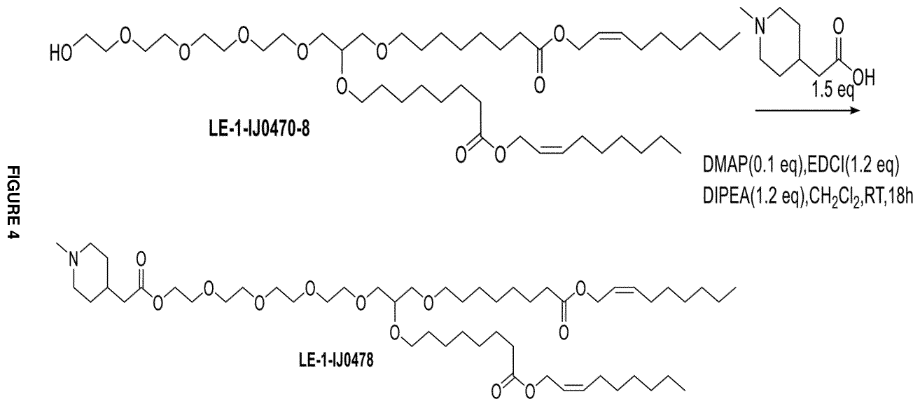

The compound VIII is prepared according to the schema of synthesis detailed on FIG. 3.

### Synthesis of LE-1-IJ0470-1

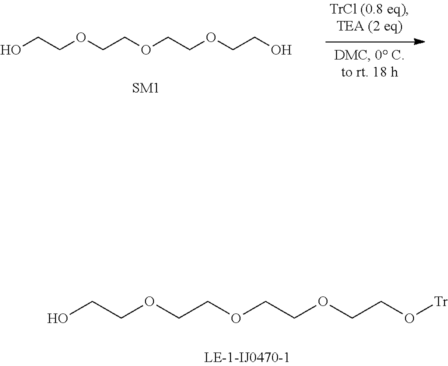

To a mixture of 2-[2-[2-(2-hydroxyethoxy) ethoxy]ethoxy] ethanol (SM1) (50 0.257 mol), N,N-dimethylpyridin-4-amine (1.57 g, 12.9 mmol) and g, [chloro (diphenyl)methyl]benzene (57.4 g, 0.206 mol) in DCM (400 mL) cooled to 0° C. was added N,N-diethylethanamine (52.1 g, 0.515 mol). The reaction mixture was stirred for 16 hrs at ambient temperature. TLC (EA: PE=2:1, Rf=0.5) indicated that a new spot was formed. The mixture was poured into water (600 mL) and extracted with DCM (2*400 mL). The organic layer was washed with water and brine, dried over Na2SO4 and concentrated. The residue was purified by flash column chromatography on silica get eluting with 1:1 EA/PE to give 2-[2-[2-(2-trityloxyethoxy) ethoxy]ethoxy] ethanol (45.7 g, 40.7% yield) as colorless oil.

1H NMR (400 MHZ, CDCl3) δ 7.46 (dt, J=3.4, 1.9 Hz, 6H), 7.32-7.26 (m, 6H), 7.24-7.18 (m, 3H), 3.73-3.63 (m, 12H), 3.61-3.56 (m, 2H), 3.27-3.21 (m, 2H), 2.56 (t, J=6.0 Hz, 1H).

### Synthesis of LE-1-IJ0470-2

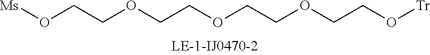

To a mixture of 2-[2-[2-(2-trityloxyethoxy) ethoxy]ethoxy] ethanol (45.7 g, 0.105 mol) and N,N-diethylethanamine (21.2 g, 0.209 mol) in DCM (600 mL) was added methanesulfonyl chloride (14.4 g, 0.126 mol) slowly at 0° C. The mixture was stirred overnight at room temperature. CH2Cl2 (400 mL) were added to the solution, and the mixture was washed with diluted HCl (1M, 1000 mL). The organic layer was further washed with Water (1000 mL) and brine (1000 mL) and dried over Na2SO4. Solvent was removed to give 2-[2-[2-(2-trityloxyethoxy) ethoxy]ethoxy]ethyl methanesulfonate (53.5 g, 99.3%) as a yellow oil.

1H NMR (400 MHZ, CDCl3) δ 7.46 (dt, J=3.4, 1.9 Hz, 6H), 7.32-7.26 (m, 6H), 7.25-7.19 (m, 3H), 4.35-4.30 (m, 2H), 3.75-3.71 (m, 2H), 3.70-3.64 (m, 10H), 3.23 (t, J=5.2 Hz, 2H), 2.98 (s, 3H).

### Synthesis of LE-1-IJ0470-1

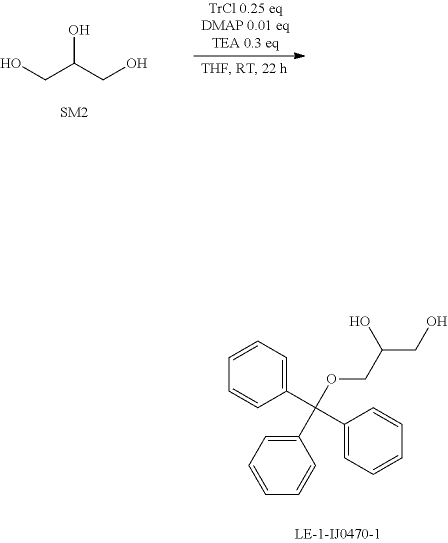 [SMILES: OCC(O)CO ; OCC(O)COC(c1ccccc1)(c1ccccc1)c1ccccc1]

To the solution of Trityl chloride (45.5 g, 163 mmol), glycerol (50 g, 543 mmol) and N,N-dimethylpyridin-4-amine (0.663 g, 5.43 mmol) in 500 mL of THF was added triethylamine (16.48 g, 163 mmol) and the mixture was stirred for 22 h at room temperature. 300 mL of ethyl acetate and 150 mL H2O were then added to the solution. The organic phase was collected and the aqueous layer was extracted with 2*300 mL ethyl acetate. The organic phases were combined, washed with 200 ml of 10% (w/v) NaHCO₃ and then 200 mL of brine, and dried on Na2SO4. The solvent was evaporated and the residue was purified on silica gel column (eluted with CH2Cl2/MeOH) to yield 3-trityloxypropane-1,2-diol as a white solid (50.4 g, 27.8% yield).

1H NMR (400 MHZ, CDCl3) δ 7.42 (dt, J=3.4, 1.9 Hz, 6H), 7.35-7.26 (m, 7H), 7.26-7.21 (m, 2H), 3.91-3.82 (m, 1H), 3.74-3.56 (m, 2H), 3.25 (m, J=15.7, 9.6, 5.2 Hz, 2H), 2.49 (d, J=5.1 Hz, 1H), 1.96 (dd, J=7.1, 5.3 Hz, 1H).

### Synthesis of LE-1-IJ0470-3

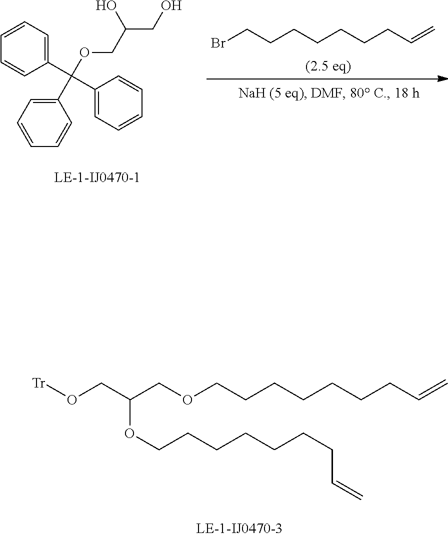

To a suspension of NaH (5.98 g, 5.0 eq) in 500 mL of anhydrous N,N-Dimethylformamide was added 3-trityloxypropane-1,2-diol (10.0 g, 1.0 eq). The mixture was heated at 80° C. for 15 minutes and cooled to room temperature. 9-bromonon-1-ene (15.3 g, 2.5 eq) in 50 mL anhydrous DMF was added dropwise to the mixture which was then heated under 80° C. for 18 h. After cooling to RT, 500 mL of H2O were added. The mixture was extracted with ethyl acetate and the organic layer was washed successively with 500 ml of 5% (w/v) NaHCO₃ and 500 mL of brine and dried on Na2SO4. The solvent was evaporated under reduced pressure and the residue was purified on a silica gel column eluted with petroleum ether/ethyl acetate (0% to 20% ethyl acetate in petroleum ether) 5% to yield a colorless oil (8.4 g, 48.2% yield).

1H NMR (400 MHZ, CDCl3) δ 7.59-7.16 (m, 17H), 5.86-5.74 (m, 3H), 4.95 (dd, J=24.2, 13.6 Hz, 6H), 3.61-3.35 (m, 12H), 3.24-3.10 (m, 2H), 2.03 (d, J=6.4 Hz, 6H), 1.46-1.24 (m, 27H).

### Synthesis of LE-1-IJ0470-4

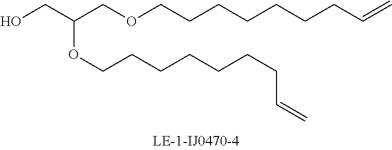 [SMILES: C=CCCCCCCCOCC(CO)OCCCCCCCC=C]

To a solution of ((2,3-bis (non-8-en-1-yloxy) propoxy) methanetriyl) tribenzene (20.0 g, 34.3 mmol) in methanol/THF (400 mL, 1/1 v/v) was added 4-methylbenzenesulfonic acid (29.5 g, 172 mmol) in one portion at room temperature and the mixture was stirred at room temperature for 18 h. TLC (4% ethyl acetate in petroleum ether) indicated that the starting material was completely disappeared. 50 mL triethylamine was added to quench the reaction and the solvent was removed under vacuum. The residue was purified by flash chromatography eluted with 20% to 30% ethyl acetate in petroleum ether (21%) to give 2,3-bis (non-8-enoxy) propan-1-ol (8.6 g, 73.6% yield) as colorless oil.

1H NMR (400 MHZ, CDCl3) δ 5.88-5.74 (m, 2H), 5.04-4.90 (m, 4H), 3.77-3.38 (m, 8H), 2.20 (s, 1H), 2.04 (q, J=7.0 Hz, 4H), 1.56 (dt, J=13.6, 6.8 Hz, 4H), 1.43-1.24 (m, 17H).

### Synthesis of LE-1-IJ0470-5

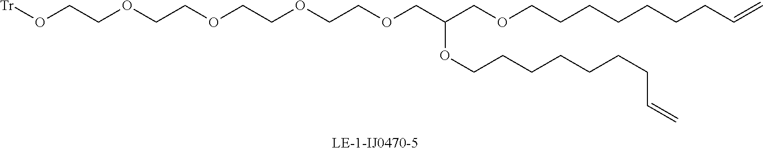

To a mixture of 2,3-bis (non-8-enoxy) propan-1-ol (8.6 g, 25.3 mmol) in 300 mL of dry THF was added NaH (60% mineral oil dispersion, 2.02 g, 50.5 mmol) and then stirred for 15 min at 800C. The reaction was cooled to room temperature and 2-[2-[2-(2-trityloxyethoxy) ethoxy]ethoxy]ethyl methanesulfonate (15.6 g, 30.3 mmol) dissolved in 50 mL of dry THF was added into above reaction. The reaction mixture was stirred at reflux (800C) overnight. The reaction mixture was cooled to room temperature, and water (200 mL) was added. EtOAc (400 mL) was added, the mixture was shaken, the layers were separated, and the organic layer was collected. The aqueous layer was extracted with EtOAc (400 mL×2). The combined organic layers were washed with brine and dried over Na2SO4. The residue was purified by flash column chromatography on silica gel eluted with ethyl acetate in petroleum ether (0-15%) (14%) to give [2-[2-[2-[2-[2,3-bis (non-8-enoxy) propoxy] ethoxy]ethoxy] ethoxy]ethoxy-diphenyl-methyl]benzene (15.7 g, 81.9% yield) as pale yellow oil.

1H NMR (400 MHZ, CDCl3) δ 7.49-7.44 (m, 7H), 7.32-7.26 (m, 7H), 7.24-7.19 (m, 4H), 5.88-5.72 (m, 2H), 5.02-4.99 (m, 1H), 4.98-4.95 (m, 1H), 4.93 (d, J=1.2 Hz, 1H), 4.91 (d, J=1.2 Hz, 1H), 3.70-3.64 (m, 12H), 3.62 (d, J=4.2 Hz, 4H), 3.59-3.39 (m, 9H), 3.23 (t, J=5.2 Hz, 2H), 2.03 (dt, J=7.8, 3.9 Hz, 4H), 1.54 (dd, J=13.1, 6.5 Hz, 4H), 1.38-1.25 (m, 16H).

### Synthesis of LE-1-IJ0470-6

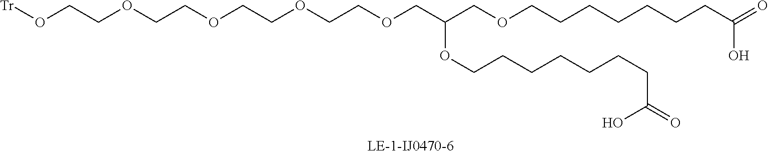

To a solution of [2-[2-[2-[2-[2,3-bis (non-8-enoxy) propoxy] ethoxy]ethoxy] ethoxy]ethoxy-diphenyl-methyl]benzene (15.7 g, 20.7 mmol) in MeCN (200 mL), CCl4 (200 mL) and water (200 mL) was added NalO4 (35.4 g, 165 mmol) and RuCl3 (0.933 g, 4.14 mmol). The reaction mixture was stirred at room temperature for 24 h. The reaction was filtered through celite and the filtrate was diluted with ethyl acetate (800 mL) and washed with 1N aq. HCl (900 mL). The organic layer was washed with Na2S2O3 solution (700 mL×2) and then dried over sodium sulfate, filtered and concentrated to give 8-[2-(7-carboxyheptoxy)-3-[2-[2-[2-(2-trityloxyethoxy) ethoxy]ethoxy] ethoxy] propoxy] octanoic acid (9.4 g, 57.2% yield) as yellow oil which was used without further purification. 1H NMR (400 MHZ, CDCl3) δ 7.48-7.44 (m, 6H), 7.29 (dd, J=10.1, 4.7 Hz, 6H), 7.24-7.20 (m, 3H), 3.72-3.38 (m, 24H), 3.23 (t, J=5.2 Hz, 2H), 2.35 (dt, J=24.3, 7.4 Hz, 4H), 1.68-1.49 (m, 8H), 1.38-1.26 (m, 12H).

### Synthesis of LE-1-IJ0470-7

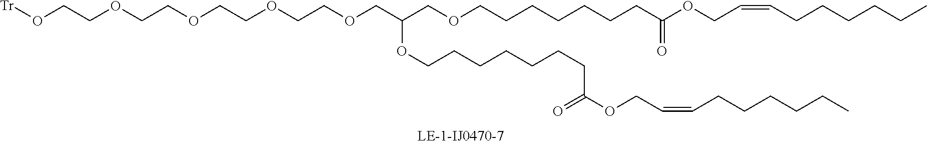

To the solution of 8-[2-(7-carboxyheptoxy)-3-[2-[2-[2-(2-trityloxyethoxy) ethoxy]ethoxy] ethoxy] propoxy] octanoic acid (1.0 g, 1.26 mmol) and (Z)-non-2-en-1-ol (0.43 g, 3.02 mmol) in dry dichloromethane (50 mL) was added DIPEA (0.98 g, 7.55 mmol), DMAP (0.062 g, 0.503 mmol) and followed by EDCl (2.56 g, 13.4 mmol) portion wise under ice-water bath. The mixture was stirred at room temperature for 18 h. The reaction was diluted with dichloromethane and washed with brine. The organic layer was dried over sodium sulfate, filtered and concentrated. The residue was purified by flash chromatography eluted with 0% to 20% (15%) ethyl acetate in petroleum ether to give [(Z)-non-2-enyl] 8-[2-[8-[(Z)-non-2-enoxy]-8-oxo-octoxy]-3-[2-[2-[2-(2-trityloxyethoxy) ethoxy]ethoxy] ethoxy] propoxy] octanoate (0.39 g, 29.7% yield) as colorless oil.

1H NMR (500 MHZ, CDCl3) δ 7.47-7.45 (m, 6H), 7.29 (t, J=7.5 Hz, 6H), 7.22 (dd, J=8.3, 6.2 Hz, 3H), 5.64 (dd, J=18.4, 7.6 Hz, 2H), 5.56-5.47 (m, 2H), 4.61 (d, J=6.8 Hz, 4H), 3.69-3.39 (m, 23H), 3.23 (t, J=5.2 Hz, 2H), 2.29 (dd, J=10.7, 4.4 Hz, 4H), 2.14-2.03 (m, 4H), 1.61-1.50 (m, 8H), 1.40-1.22 (m, 28H), 0.88 (t, J=6.9 Hz, 6H).

### Synthesis of LE-1-IJ0470-8

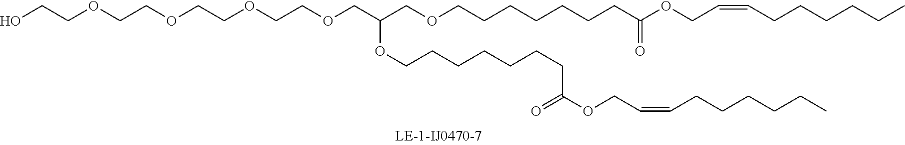 [SMILES: CCCCCC/C=C\COC(=O)CCCCCCCOCC(COCCOCCOCCOCCO)OCCCCCCCC(=O)OC/C=C\CCCCCC]

To a solution of [(Z)-non-2-enyl] 8-[2-[8-[(Z)-non-2-enoxy]-8-oxo-octoxy]-3-[2-[2-[2-(2-Trityloxyethoxy) ethoxy]ethoxy] ethoxy] propoxy] octanoate (0.39 g, 0.374 mmol) in methanol/THF (40 mL, 1/1 v/v) was added p-Toluenesulfonic acid (0.322 g, 1.87 mmol) in one portion at room temperature and the mixture was stirred at room temperature for 2 h. TLC (30% ethyl acetate in petroleum ether) indicated that the starting material was completely disappeared. 5 mL triethylamine was added to quench the reaction and the solvent was removed under vacuum. The residue was purified by flash chromatography eluted with 0% to 20% (10%) methanol in DCM to give 2,3-bis (non-8-enoxy) propan-1-ol (0.270 g, 90.2% yield) as colorless oil.

1H NMR (400 MHZ, CDCl3) δ 5.70-5.59 (m, 2H), 5.58-5.47 (m, 2H), 4.62 (d, J=6.7 Hz, 4H), 3.77-3.37 (m, 27H), 2.70 (s, 1H), 2.37-2.24 (m, 4H), 2.10 (q, J=6.8 Hz, 4H), 1.66-1.50 (m, 8H), 1.41-1.20 (m, 28H), 0.88 (t, J=6.9 Hz, 6H).

### Synthesis of Compound VIII

To the solution of [(Z)-non-2-enyl] 8-[3-[2-[2-[2-(2-hydroxyethoxy) ethoxy]ethoxy] ethoxy]-2-[8-[(Z)-non-2-enoxy]-8-oxo-octoxy] propoxy] octanoate (570 mg, 0.711 mmol) and 1-methylpiperidine-4-carboxylic acid (0.153 g, 1.07 mmol) in dry dichloromethane (15 mL) was added DIPEA (0.110 g, 0.854 mmol), DMAP (0.009 g, 0.071 mmol) and EDCl (0.164 g, 0.854 mmol) under ice bath. The mixture was stirred at room temperature for 18 h. The reaction was diluted with dichloromethane and washed with brine. The organic layer was dried over sodium sulfate, filtered and concentrated. The residue was purified by flash chromatography eluted with 0% to 20% MeOH in DCM to give 2-[2-[2-[2-[2-[8-[(Z)-non-2-enoxy]-8-oxo-octoxy]-3-[8-[(Z)-oct-2-enoxy]-8-oxo-octoxy] propoxy] ethoxy]ethoxy] ethoxy]ethyl 1-methylpiperidine-4-carboxylate (0.414 g, 63.8% yield) as colorless oil.

1H NMR (400 MHZ, CDCl3) δ 5.64 (dt, J=10.9, 7.4 Hz, 2H), 5.52 (dt, J=11.0, 6.8 Hz, 2H), 4.62 (d, J=6.8 Hz, 4H), 4.27-4.21 (m, 2H), 3.72-3.61 (m, 14H), 3.58-3.39 (m, 9H), 2.85 (d, J=11.2 Hz, 2H), 2.39-2.26 (m, 8H), 2.10 (dd, J=14.0, 6.9 Hz, 5H), 1.94 (s, 2H), 1.82 (d, J=10.6 Hz, 3H), 1.66-1.50 (m, 9H), 1.41-1.24 (m, 30H), 0.88 (t, J=6.9 Hz, 6H).

### Example 4: Synthesis of [(Z)-non-2-enyl] 8-[3-[2-[2-[2-[2-[3-(4-methylpiperazin-1-yl) propanoyloxy] ethoxy]ethoxy] ethoxy]ethoxy]-2-[8-[(Z)-non-2-enoxy]-8-oxooctoxy] propoxy] octanoate (compound IX)

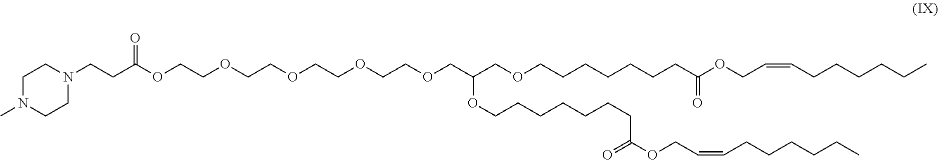 [SMILES: CCCCCC/C=C\COC(=O)CCCCCCCOCC(COCCOCCOCCOCCOC(=O)CCN1CCN(C)CC1)OCCCCCCCC(=O)OC/C=C\CCCCCC]

The compound IX is prepared from the compound LE616IJ0470-8 (for the synthesis of LE616IJ0470-8 see example 3).

To the solution of [(Z)-non-2-enyl] 8-[3-[2-[2-[2-(2-hydroxyethoxy) ethoxy]ethoxy] ethoxy]-2-[8-[(Z)-non-2-enoxy]-8-oxo-octoxy] propoxyloctanoate (400 mg, 0.500 mmol) and 3-(4-methylpiperazin-1-yl) propanoic acid (0.129 g, 0.750 mmol) in dry dichloromethane (10 mL) then added DIPEA (0.078 g, 0.600 mmol), DMAP (0.006 g, 0.050 mmol) and under ice bath added EDCl (0.115 g, 0.600 mmol). The mixture was stirred at room temperature for 18 h. The reaction was diluted with dichloromethane and washed with brine. The organic layer was dried over sodium sulfate, filtered and concentrated. The residue was purified by flash chromatography eluted with 0% to 20% (9%) MeOH in DCM to give [(Z)-non-2-enyl] 8-[3-[2-[2-[2-[2-[3-(4-methylpiperazin-1-yl) propanoyloxy] ethoxy]ethoxy] ethoxy]ethoxy]-2-[8-[(Z)-non-2-enoxy]-8-oxo-octoxy] propoxy] octanoate (0.378 g, 79.2% yield) as light yellow oil.

1H NMR (400 MHZ, CDCl3) δ 5.69-5.47 (m, 4H), 4.62 (d, J=6.8 Hz, 4H), 4.26-4.21 (m, 2H), 3.75-3.60 (m, 17H), 3.60-3.38 (m, 11H), 2.72 (t, J=7.4 Hz, 2H), 2.57-2.49 (m, 6H), 2.33-2.27 (m, 8H), 2.10 (q, J=6.9 Hz, 4H), 1.67-1.51 (m, 10H), 1.41-1.09 (m, 32H), 0.88 (t, J=6.8 Hz, 6H).

### Example 5: Synthesis of [(Z)-non-2-enyl] 8-[3-[2-[2-[2-[2-[2-(1-methyl-4-piperidyl) acetyl] oxyethoxy]ethoxy] ethoxy]ethoxy]-2-[8-[(Z)-non-2-enoxy]-8-oxooctoxy] propoxy] octanoate (compound X)

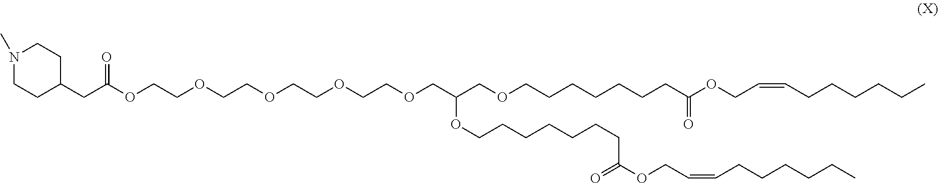 [SMILES: CCCCCC/C=C\COC(=O)CCCCCCCOCC(COCCOCCOCCOCCOC(=O)CC1CCN(C)CC1)OCCCCCCCC(=O)OC/C=C\CCCCCC]

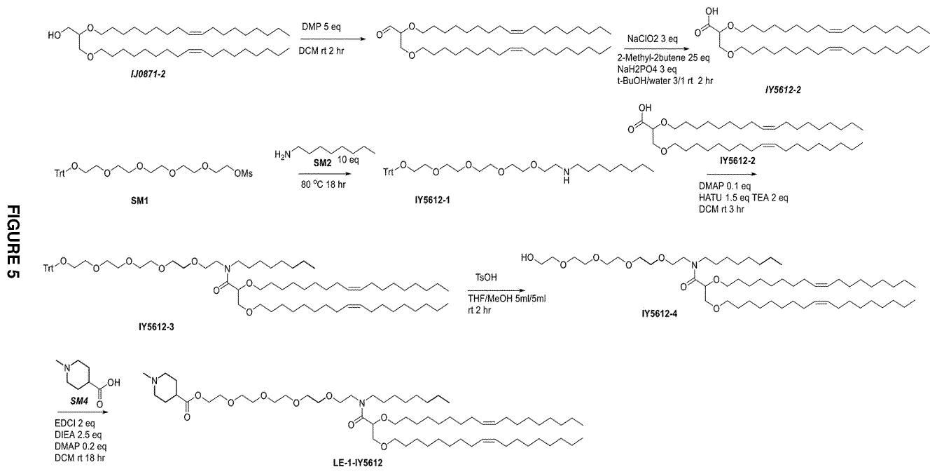

The compound X is prepared from the schema of synthesis of FIG. 4.

To the solution of [(Z)-non-2-enyl] 8-[3-[2-[2-[2-(2-hydroxyethoxy) ethoxy]ethoxy] ethoxy]-2-[8-[(Z)-non-2-enoxy]-8-oxo-octoxy] propoxy] octanoate (400 mg, 0.500 mmol) and 2-(1-methyl-4-piperidyl) acetic acid (0.118 g, 0.749 mmol) in dry dichloromethane (10 mL) then added DIPEA (0.078 g, 0.600 mmol), DMAP (0.006 g, 0.050 mmol) and under ice bath added EDCl (0.115 g, 0.600 mmol). The mixture was stirred at room temperature for 18 h. The reaction was diluted with dichloromethane and washed with brine. The organic layer was dried over sodium sulfate, filtered and concentrated. The residue was purified by flash chromatography eluted with 0% to 20% (9%) MeOH in DCM to give [(Z)-non-2-enyl] 8-[3-[2-[2-[2-[2-[2-(1-methyl-4-piperidyl) acetyl] oxyethoxy]ethoxy] ethoxy]ethoxy]-2-[8-[(Z)-non-2-enoxy]-8-oxo-octoxy] propoxy] octanoate (0.271 g, 57.7% yield) as light yellow oil.

1H NMR (400 MHZ, CDCl3) δ 5.64 (dt, J=10.9, 7.5 Hz, 2H), 5.57-5.47 (m, 2H), 4.62 (d, J=6.8 Hz, 4H), 4.26-4.20 (m, 2H), 3.76-3.60 (m, 16H), 3.60-3.37 (m, 11H), 3.02 (s, 2H), 2.43 (s, 3H), 2.28 (dd, J=24.3, 16.8 Hz, 9H), 2.10 (dd, J=14.3, 7.1 Hz, 4H), 1.80 (d, J=13.8 Hz, 4H), 1.65-1.51 (m, 11H), 1.40-1.20 (m, 31H), 0.88 (t, J=6.8 Hz, 6H).

### Example 6: Synthesis of 2-[2-[2-[2-[2-[2,3-bis [(Z)-octadec-9-enoxy] propanoyl-octylamino]ethoxy]ethoxy] ethoxy]ethoxy]ethyl 1-methylpiperidine-4-carboxylate (compound XI)

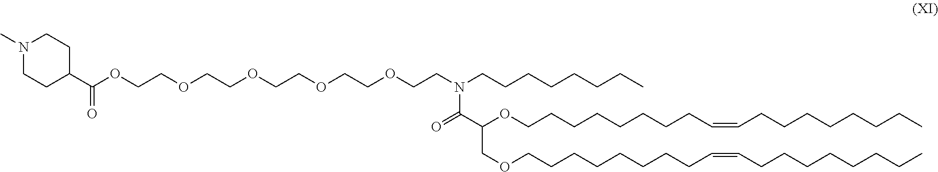 [SMILES: CCCCCCCC/C=C\CCCCCCCCOCC(OCCCCCCCC/C=C\CCCCCCCC)C(=O)N(CCCCCCCC)CCOCCOCCOCCOCCOC(=O)C1CCN(C)CC1]

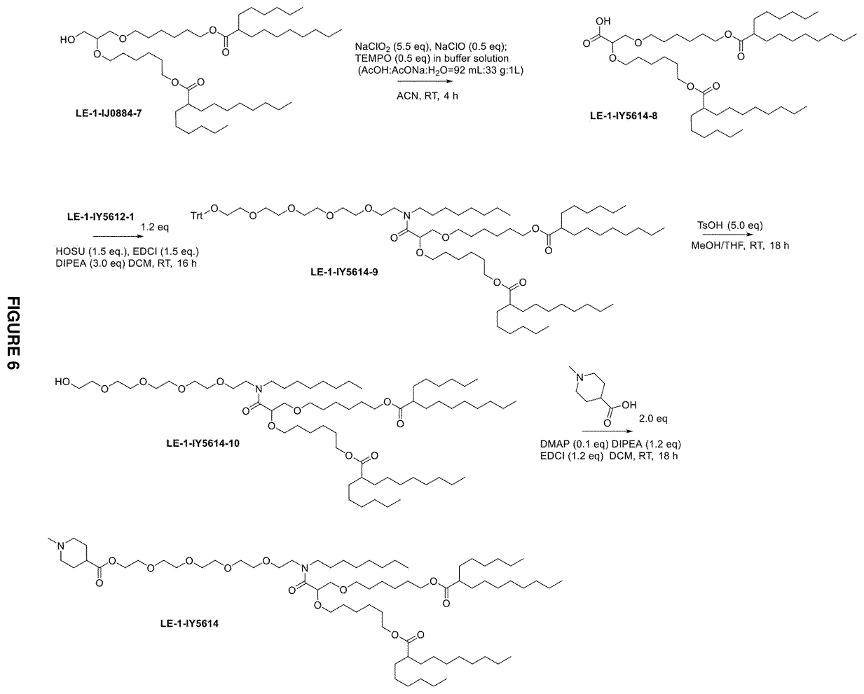

The compound XI is prepared according to the schema of synthesis of FIG. 5.

### Synthesis of the intermediate

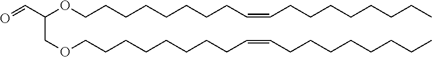 [SMILES: CCCCCCCC/C=C\CCCCCCCCOCC(C=O)OCCCCCCCC/C=C\CCCCCCCC]

To a solution of 2,3-bis [(Z)-octadec-9-enoxy] propan-1-ol (5 g, 8.43 mmol) in DCM (100 ml) at 0° C. was added Dess-Martin Periodinane (5.36 g, 12.6 mmol). The reaction was stirred at room temperature for 2 h. TLC indicated that the starting material was consumed. The mixture was filtered and concentrated. The crude was diluted with ethyl acetate (50 ml), washed with Na2S2O3/NaHCO₃ (25 ml/25 ml×3), brine (50 ml) and dried over Na2SO4. The organic was concentrated to give 2,3-bis [(Z)-octadec-9-enoxy] propanal (5 g, 8.29 mmol, yield 98.3%) as a colorless oil which was taken into next step directly.

1H NMR (500 MHZ, CDCl3) δ 9.72 (d, J=1.1 Hz, 1H), 5.40-5.31 (m, 3H), 3.84-3.54 (m, 5H), 3.49-3.39 (m, 2H), 2.04-1.92 (m, 7H), 1.63 (dd, J=14.2, 7.1 Hz, 2H), 1.58-1.53 (m, 2H), 1.34-1.24 (m, 44H), 0.88 (t, J=6.9 Hz, 6H).

### Synthesis of the intermediate LE-1-IY5612-2

2,3-bis [(Z)-octadec-9-enoxy] propanal (5 g, 8.46 mmol) was dissolved in t-BuOH: H2O (3:1.60 mL), containing NaH2PO4 (3.05 g, 25.4 mmol), 2-methy-2-butene (17.8 ml) and sodium chlorite (2.3 g, 25.4 mmol). The reaction was stirred for 1 h at room temperature and was diluted with ethyl acetate (150 ml) washed with water (200 ml×2). The aqueous layer was extracted with ethyl acetate (50 ml). The combined extract was dried over sodium sulfate and concentrated to afford 2,3-bis [(Z)-octadec-9-enoxy] propanoic acid (4.7 g, 7.6 mmol, yield 89.7%) as colorless oil.

1H NMR (500 MHZ, CDCl3) δ 5.43-5.31 (m, 3H), 4.04 (dd, J=4.9, 3.2 Hz, 1H), 3.81-3.41 (m, 6H), 2.04-1.92 (m, 6H), 1.65-1.55 (m, 4H), 1.34-1.24 (m, 44H), 0.88 (t, J=6.9 Hz, 6H).

### Synthesis of the intermediate LE-1-IY5612-1

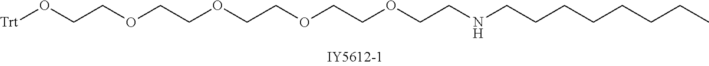

2-[2-[2-[2-(2-trityloxyethoxy) ethoxy]ethoxy] ethoxy]ethyl methanesulfonate (11 g, 17.7 mmol) was added to octan-1-amine (44 ml), and the mixture was stirred at 80° C. for 18 hr. LCMS showed the SM was consumed and the product was formed. The mixture was diluted with ethyl acetate (EA) (500 ml), washed with water (500 ml×2), brine (500 ml) and dried over Na2SO4. The organic layer was concentrated and purified by flash chromatography column (10% MeOH in DCM) to give N-[2-[2-[2-[2-(2-trityloxyethoxy) ethoxy]ethoxy] ethoxy]ethyl] octan-1-amine (10 g, 16.6 mmol, yield 93.4%) as a yellow oil.

1H NMR (500 MHZ, CDCl3) δ 7.48-7.43 (m, 6H), 7.29 (dd, J=10.3, 4.8 Hz, 6H), 7.25-7.20 (m, 3H), 3.75-3.53 (m, 16H), 3.24 (t, J=5.2 Hz, 2H), 2.86 (t, J=5.1 Hz, 2H), 2.70-2.63 (m, 2H), 1.62-1.51 (m, 2H), 1.26 (d, J=5.0 Hz, 10H), 0.87 (t, J=6.9 Hz, 3H).

### Synthesis of the intermediate LE-1-IY5612-3

To a solution of N-[2-[2-[2-[2-(2-trityloxyethoxy) ethoxy]ethoxy] ethoxy]ethyl] octan-1-amine (1.38 g, 1.98 mmol) in DCM (20 ml) was added 2,3-bis [(Z)-octadec-9-enoxy] propanoic acid (1 g, 1.65 mmol), 4-Dimethylaminopyridine (20 mg), O—(7-Azabenzotriazol-1-yl)-N,N,N′,N′-tetramethyluronium hexafluorophosphate (940 mg, 2.47 mmol) and TEA (333 mg, 3.29 mmol). The mixture was stirred at 25° C. for 18 h. Then the mixture was diluted with DCM (50 ml), washed with water (250 mL×2), brine (250 ml) and dried over Na2SO4. The organic layer was purified by flash chromatography column (5% MeOH in DCM) to give 2,3-bis [(Z)-octadec-9-enoxy]-N-octyl-N-[2-[2-[2-[2-(2-trityloxyethoxy) ethoxy]ethoxy] ethoxy]ethyl]propanamide (1.15 g, 0.95 mmol, yield 57.9%) as a yellow oil.

1H NMR (500 MHZ, CDCl3) δ 7.46 (d, J=7.6 Hz, 6H), 7.28 (dd, J=12.5, 4.6 Hz, 6H), 7.22 (t, J=7.2 Hz, 3H), 5.39-5.31 (m, 3H), 4.43-4.28 (m, 1H), 3.83-3.08 (m, 28H), 1.99 (dd, J=15.5, 9.2 Hz, 7H), 1.56-1.19 (m, 60H), 0.92-0.84 (m, 9H).

### Synthesis of the intermediate LE-1-IY5612-4

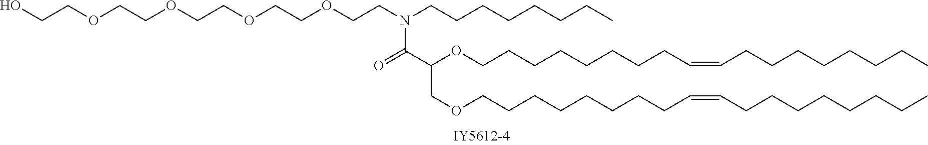 [SMILES: CCCCCCCC/C=C\CCCCCCCCOCC(OCCCCCCCC/C=C\CCCCCCCC)C(=O)N(CCCCCCCC)CCOCCOCCOCCOCCO]

To a solution of 2,3-bis [(Z)-octadec-9-enoxy]-N-octyl-N-[2-[2-[2-[2-(2-trityloxyethoxy) ethoxy]ethoxy] ethoxy]ethyl]propanamide (1.15 g, 0.97 mmol) in THF/MeOH (10 ml, 1/1) was added Toluene-4-sulfonic acid (371 mg, 1.95 mmol). The mixture was stirred at 25° C. for 2 hr. The mixture diluted with ethyl acetate (EA) (50 ml), washed with NaHCO₃ (50 ml), brine (50 ml) and dried over Na2SO4. The organic layer was concentrated and purified by flash chromatography column (10 MeOH % in DCM) to give N-[2-[2-[2-[2-(2-hydroxyethoxy) ethoxy]ethoxy] ethoxy]ethyl]-2,3-bis [(Z)-octadec-9-enoxy]-N-octyl-propanamide (730 mg, 0.78 mmol, yield 79.9%) as a colorless oil.

1H NMR (400 MHZ, CDCl3) δ 5.43-5.31 (m, 3H), 4.45-4.30 (m, 1H), 4.00-3.19 (m, 28H), 2.10-1.90 (m, 8H), 1.67-1.16 (m, 60H), 0.95-0.82 (m, 9H).

### Synthesis of the Compound XI

To a solution of 1-methylpiperidine-4-carboxylic acid (111 mg, 0.78 mmol) in DCM (10 ml) was added N-[2-[2-[2-[2-(2-hydroxyethoxy) ethoxy]ethoxy] ethoxy]ethyl]-2,3-bis [(Z)-octadec-9-enoxy]-N-octyl-propanamide (730 mg, 0.78 mmol), DIEA (251 mg, 1.94 mmol), DMAP (19 mg) and EDC HCl (298 mg, 1.56 mmol). The mixture was stirred at 25° C. for 18 hr. Then the mixture was diluted with Ethyl Acetate (50 ml), washed with water (50 ml), brine (50 ml) and dried over Na2SO4. The organic layer was concentrated and the residue was purified by flash chromatography column (50% Ethyl Acetate in PE) to give 2-[2-[2-[2-[2-[2,3-bis [(Z)-octadec-9-enoxy] propanoyl-octyl-amino]ethoxylethoxylethoxy]ethoxy]ethyl 1-methylpiperidine-4-carboxylate (318 mg, 0.29 mmol, yield 37.6%) as a colorless oil.

1H NMR (400 MHZ, CDCl3) δ 5.35 (dd, J=12.4, 7.2 Hz, 3H), 4.44-4.30 (m, 1H), 4.29-4.19 (m, 2H), 3.74-3.23 (m, 26H), 2.83 (s, 2H), 2.30 (s, 3H), 2.11-1.68 (m, 14H), 1.62-1.15 (m, 60H), 0.94-0.82 (m, 9H).

### Example 7: Synthesis of 2-[2-[2-[2-[2-[2,3-bis [6-(2-hexyldecanoyloxy) hexoxy] propanoyl-octylamino]ethoxy]ethoxy] ethoxy]ethoxy]ethyl 1-methylpiperidine-4-carboxylate (compound XII)

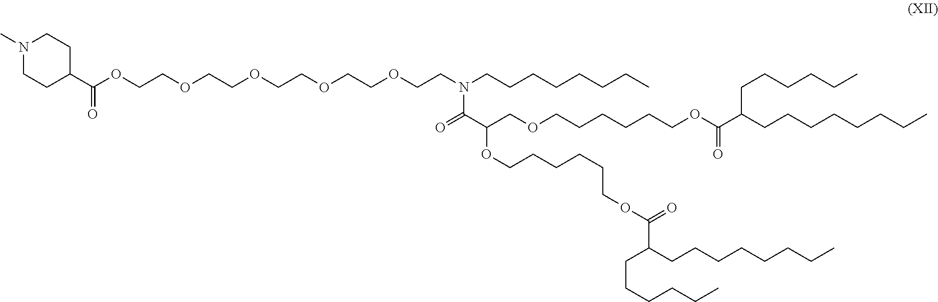 [SMILES: CCCCCCCCC(CCCCCC)C(=O)OCCCCCCOCC(OCCCCCCOC(=O)C(CCCCCC)CCCCCCCC)C(=O)N(CCCCCCCC)CCOCCOCCOCCOCCOC(=O)C1CCN(C)CC1]

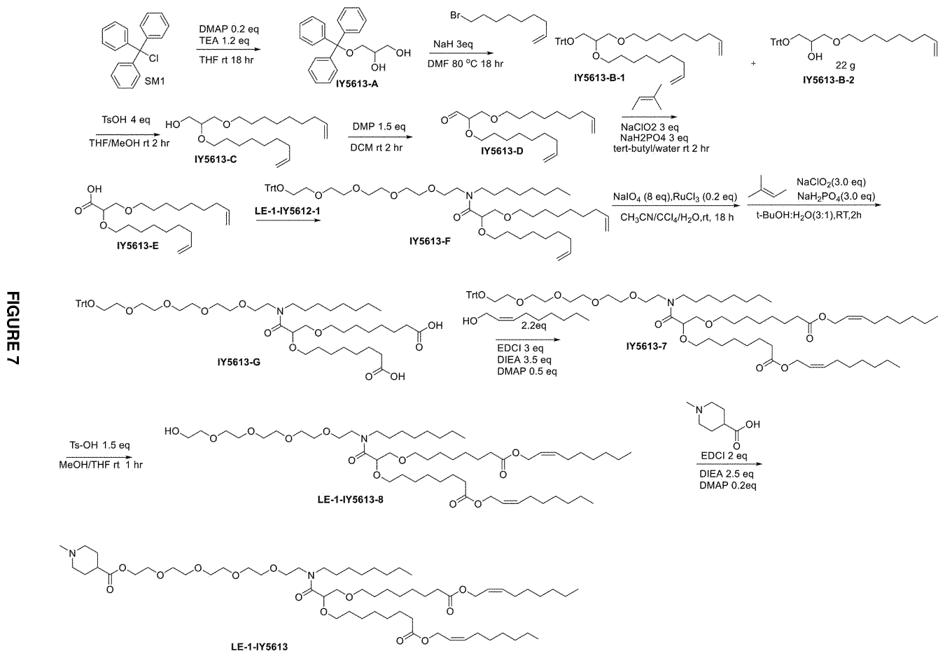

The compound XII is prepared according to the schema of synthesis of FIG. 6.

### Synthesis of the intermediate LE-1-IY5614-8

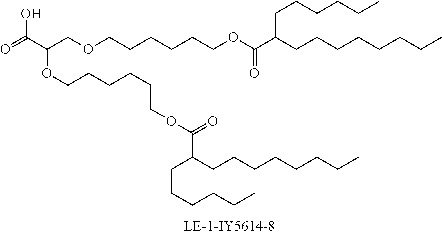 [SMILES: CCCCCCCCC(CCCCCC)C(=O)OCCCCCCOCC(OCCCCCCOC(=O)C(CCCCCC)CCCCCCCC)C(=O)O]

To a solution of 6-[2-[6-(2-hexyldecanoyloxy) hexoxy]-3-hydroxy-propoxy] hexyl 2-hexyldecanoate (1.5 g, 1.95 mmol) in Acetonitrile (AcCN, 20 mL) and PH=4-buffer solution (10 mL, AcOH: AcONa: water=92 mL: 33 g: 1000 mL) was added sodium chlorite (0.97 g, 10.7 mmol) and sodium hypochlorite (0.0726 g, 0.975 mmol) and followed by TEMPO ((2,2,6,6-tetramethylpiperidin-1-yl)oxyl) (0.152 g, 0.0975 mmol). The reaction became black and stirred at 20° C. for 4 hr. LCMS indicated a clean reaction. The reaction was quenched with 20 drops of methanol and was poured into water (40 mL) and extracted with ethyl acetate (60 ml×3). The organic layers were combined, washed with brine (60 mL), dried over anhydrous sodium sulfate, filtered and concentrated. The residue was purified through flash chromatography eluted with 5% to 10% methanol in dichloromethane to give 2,3-bis [6-(2-hexyldecanoyloxy) hexoxy] propanoic acid (1.23 g, 80.5% yield) as light yellow oil.

1H NMR (400 MHZ, CDCl3) δ 4.11-4.02 (m, 5H), 3.82-3.41 (m, 6H), 2.36-2.26 (m, 2H), 1.70-1.52 (m, 12H), 1.47-1.34 (m, 12H), 1.25 (s, 40H), 0.87 (dd, J=6.9, 6.2 Hz, 12H).

### Synthesis of the intermediate LE-1-IY5614-9

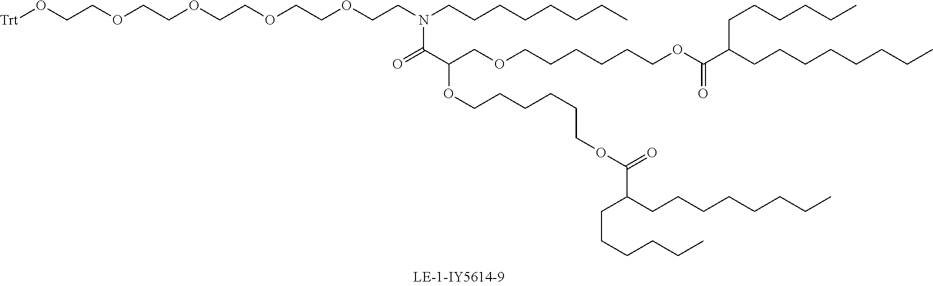

A mixture of 2,3-bis [6-(2-hexyldecanoyloxy) hexoxy] propanoic acid (1.23 g, 1.57 mmol), EDC HCl (0.452 g, 2.36 mmol), N-Hydroxysuccinimide (0.271 g, 2.36 mmol) in DCM (20 mL). The reaction mixture was stirred for 2 h at RT. Then N-[2-[2-[2-[2-(2-trityloxyethoxy) ethoxy]ethoxy] ethoxy]ethyl] octan-1-amine (1.12 g, 1.88 mmol) and DIEA (0.61 g, 4.71 mmol) were added. The mixture was stirred for 16 h at RT. The mixture was poured into DCM (200 mL) and washed with NaHCO₃ solution and brine, dried over Na2SO4 and concentrated. The residue was purified by column chromatography on silica gel eluting with 2-5% methanol in dichloromethane (3%) to afford 6-[2-[6-(2-hexyldecanoyloxy) hexoxy]-3-[octyl-[2-[2-[2-[2-(2-trityloxyethoxy) ethoxy]ethoxy] ethoxy]ethyl]amino]-3-oxo-propoxy] hexyl 2-hexyldecanoate (1.27 g, 59.6% yield) as light yellow oil.

1H NMR (400 MHZ, CDCl3) δ 7.69-6.99 (m, 15H), 4.46-4.27 (m, 1H), 4.06 (q, J=6.6 Hz, 4H), 3.89-3.19 (m, 26H), 2.35-2.26 (m, 2H), 1.75-1.33 (m, 28H), 1.25 (s, 50H), 0.88 (dd, J=8.3, 4.3 Hz, 15H).

### Synthesis of the intermediate LE-1-IY5614-8-10

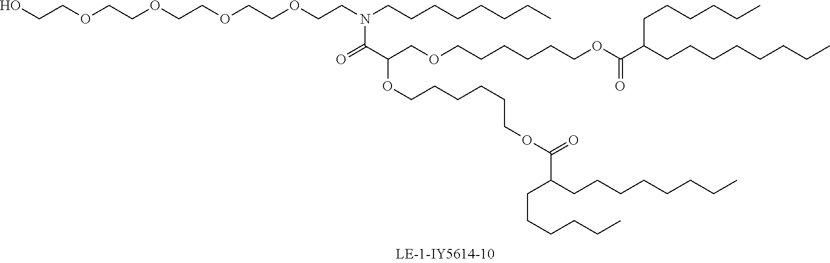 [SMILES: CCCCCCCCC(CCCCCC)C(=O)OCCCCCCOCC(OCCCCCCOC(=O)C(CCCCCC)CCCCCCCC)C(=O)N(CCCCCCCC)CCOCCOCCOCCOCCO]

To a solution of 6-[2-[6-(2-hexyldecanoyloxy) hexoxy]-3-[octyl-[2-[2-[2-[2-(2-trityloxyethoxy) ethoxy]ethoxy] ethoxy]ethyl]amino]-3-oxo-propoxy] hexyl 2-hexyldecanoate (1.27 g, 0.936 mmol) in methanol/THF (30 mL, 1/1 v/v) was added 4-methylbenzenesulfonic acid (0.89 g, 4.68 mmol) in one portion at room temperature and the mixture was stirred at room temperature for 18 h. TLC (4% ethyl acetate in petroleum ether) indicated that the starting material was disappeared completely. 30 mL triethylamine was added to quench the reaction and the solvent was removed under vacuum. The residue was purified by flash chromatography eluted with 0% to 5% MeOH in DCM (4%) to give 6-[2-[6-(2-hexyldecanoyloxy) hexoxy]-3-[2-[2-[2-[2-(2-hydroxyethoxy) ethoxy]ethoxy] ethoxy]ethyl-octyl-amino]-3-oxo-propoxy] hexyl-hexyldecanoate. (0.78 g, 74.8% yield) as colorless oil.

1H NMR (400 MHZ, CDCl3) δ 4.46-4.29 (m, 1H), 4.05 (td, J=6.6, 2.1 Hz, 4H), 3.78-3.24 (m, 28H), 2.35-2.25 (m, 2H), 1.69-1.32 (m, 28H), 1.25 (s, 50H), 0.87 (dd, J=6.7, 5.8 Hz, 15H).

### Synthesis of the compound XII

To the solution of 6-[2-[6-(2-hexyldecanoyloxy) hexoxy]-3-[2-[2-[2-[2-(2-hydroxyethoxy) ethoxy]ethoxy] ethoxy]ethyl-octyl-amino]-3-oxo-propoxy] hexyl 2-hexyldecanoate (0.4 g, 0.359 mmol) and 1-methylpiperidine-4-carboxylic acid (0.103 g, 0.718 mmol) in dry dichloromethane (10 mL) were added DIPEA (0.056 g, 0.431 mmol), DMAP (0.004 g, 0.036 mmol) and followed by under ice bath EDCl (0.08 g, 0.431 mmol) portionwise. The mixture was stirred at room temperature for 18 h. The reaction was diluted with dichloromethane and washed with brine. The organic layer was dried over sodium sulfate, filtered and concentrated. The residue was purified by flash chromatography eluted with 0% to 20% methanol in dichloromethane to give 2-[2-[2-[2-[2-[2,3-bis [6-(2-hexyldecanoyloxy) hexoxy] propanoyl-octyl-amino]ethoxy]ethoxy] ethoxy]ethoxy]ethyl 1-methylpiperidine-4-carboxylate (0.278 g, 62.5% yield) as colorless oil.

1H NMR (400 MHZ, CDCl3) δ 4.43-4.30 (m, 1H), 4.27-4.21 (m, 2H), 4.05 (td, J=6.6, 2.1 Hz, 4H), 3.72-3.29 (m, 26H), 2.84 (d, J=11.2 Hz, 2H), 2.38-2.23 (m, 6H), 2.05 (s, 2H), 1.95 (d, J=15.4 Hz, 2H), 1.86-1.74 (m, 3H), 1.67-1.52 (m, 14H), 1.46-1.33 (m, 12H), 1.25 (s, 50H), 0.87 (t, 15H).

### Example 8: Synthesis of -[2-[2-[2-[2-[2,3-bis [8-[(Z)-non-2-enoxy]-8-oxo-octoxy] propanoyl-octylamino]ethoxy]ethoxy] ethoxy]ethoxy]ethyl 1-methylpiperidine-4-carboxylate (compound XIII)

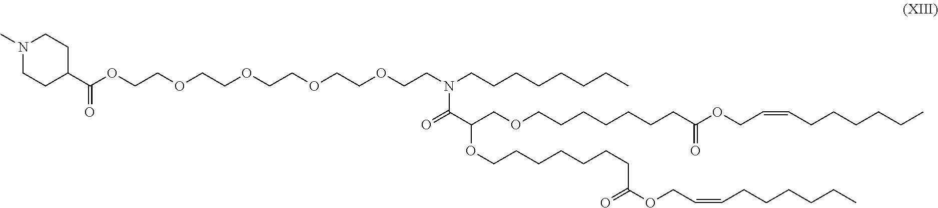 [SMILES: CCCCCC/C=C\COC(=O)CCCCCCCOCC(OCCCCCCCC(=O)OC/C=C\CCCCCC)C(=O)N(CCCCCCCC)CCOCCOCCOCCOCCOC(=O)C1CCN(C)CC1]

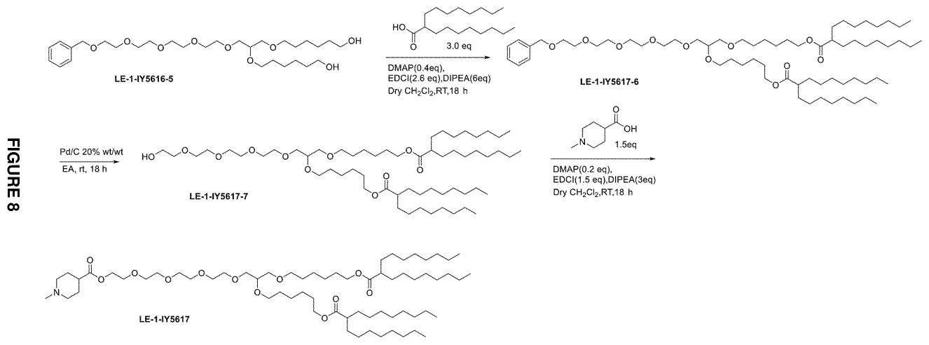

The compound XIII is prepared according to the schema of synthesis of FIG. 7.

### Synthesis of the LE-1-IY5613

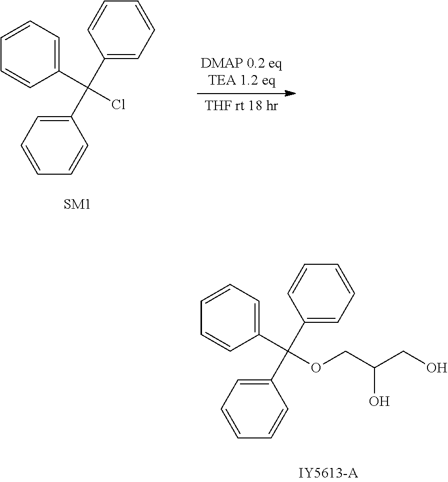 [SMILES: ClC(c1ccccc1)(c1ccccc1)c1ccccc1 ; OCC(O)COC(c1ccccc1)(c1ccccc1)c1ccccc1]

Trityle chloride (96.6 g, 347 mmol), glycerol (129 g, 1400 mmol) and N,N-dimethylpyridin-4-amine (966 mg, 7.91 mmol) were dissolved in 300 ml of THF. After addition of triethylamine (42.5 g, 420 mmol), the mixture was stirred for 22 h at room temperature. 300 mL of ethyl acetate and 300 mL H2O were then added to the solution. The organic phase was collected and extracted with 2*300 mL ethyl acetate. The organic phases were combined, washed with 200 ml of 10% (w/v) NaHCO₃ and then 200 ml of brine, and dried on Na2SO4. The solvent was evaporated and the residual oil was recrystallized in benzene/hexane. The obtained product was further purified on silica gel column (elution gradient CH2Cl2/MeOH, 5% MeOH in DCM) to give 3-trityloxypropane-1,2-diol as a white solid (67.9 g, 203 mmol, 63.7% yield).

1H NMR (400 MHZ, CDCl3) δ 7.42 (d, J=7.8 Hz, 6H), 7.31 (t, J=7.5 Hz, 6H), 7.24 (dd, J=8.9, 5.5 Hz, 3H), 3.85 (s, 1H), 3.62 (dt, J=11.3, 8.5 Hz, 2H), 3.24 (ddd, J=15.6, 9.6, 5.3 Hz, 2H), 2.53 (d, J=3.7 Hz, 1H), 2.02 (s, 1H).

### Synthesis of IY5613-B-1

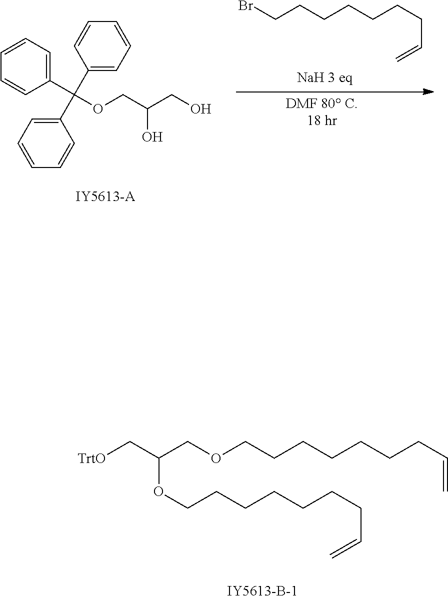

To a suspension of NaH (24.4 g, 5.0 eq) in 500 mL of anhydrous N,N-Dimethylformamide was added 3-trityloxypropane-1,2-diol (67.9 g, 1.0 eq). The mixture was heated at 80° C. for 1 hr and cooled to room temperature. 9-bromonon-1-ene (104 g, 2.5 eq) in 50 mL anhydrous DMF was added dropwise to the mixture at 0° C. which was then heated under 80° C. for 18 h. After cooling to RT, 1000 mL of H2O were added to wash successively with 500 mL of 5% (w/v) NaHCO₃ and 500 mL of brine and dried on Na2SO4. The solvent was evaporated under reduced pressure and the resulting oil was purified on a silica gel column eluted with petroleum ether/ethyl acetate (0% to 20% ethyl acetate in petroleum ether) to yield a colorless oil (20 g, 16.6% yield).

1H NMR (500 MHZ, CDCl3) δ 7.46 (dd, J=5.2, 3.4 Hz, 6H), 7.31-7.26 (m, 6H), 7.25-7.20 (m, 3H), 5.80 (ttd, J=13.1, 6.7, 2.7 Hz, 2H), 5.02-4.89 (m, 4H), 3.59-3.35 (m, 7H), 3.21-3.11 (m, 2H), 2.08-1.98 (m, 4H), 1.55-1.24 (m, 20H).

### Synthesis of IY5613-C

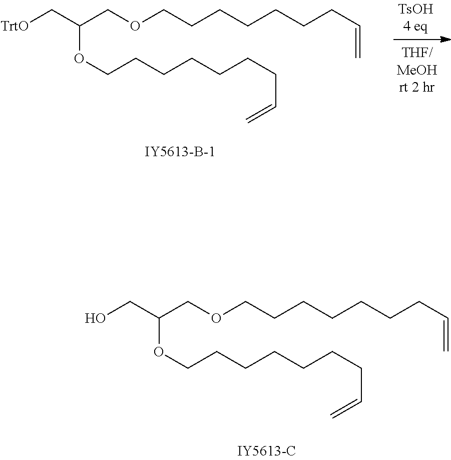

To a solution of [2,3-bis (non-8-enoxy) propoxy-diphenyl-methyl]benzene (27 g, 46.3 mmol) in THF/MeOH (100 ml/100 ml) was added Toluene-4-sulfonic acid (6.53 g, 34.3 mmol). The mixture was stirred at 25° C. for 2 hr. Then the mixture was concentrated and diluted with ethyl acetate (100 ml), washed with NaHCO₃ aq. (100×2 ml), brine (100 ml) and dried over Na2SO4. The organic layer was concentrated and purified by flash chromatography column (20% EA in PE) to give 2,3-bis (non-8-enoxy) propan-1-ol (12.8 g, 35.7 mmol, yield 77.1%) as a colorless oil.

1H NMR (400 MHZ, CDCl3) δ 5.81 (ddt, J=16.9, 10.2, 6.7 Hz, 2H), 5.06-4.88 (m, 4H), 3.76-3.41 (m, 9H), 2.04 (q, J=6.9 Hz, 4H), 1.56 (dt, J=13.6, 6.9 Hz, 4H), 1.40-1.27 (m, 16H).

### Synthesis of IY5613-D

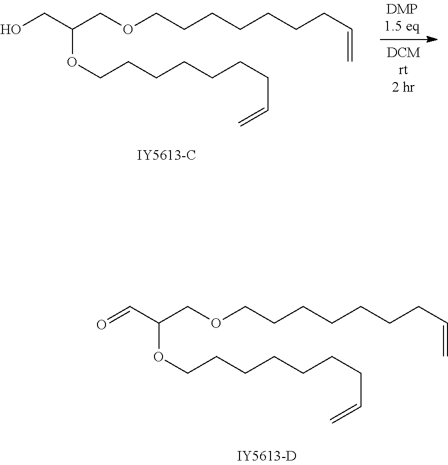 [SMILES: C=CCCCCCCCOCC(CO)OCCCCCCCC=C ; C=CCCCCCCCOCC(C=O)OCCCCCCCC=C]

To a solution of 2,3-bis (non-8-enoxy) propan-1-ol (12.8 g, 37.6 mmol) in DCM (200 ml) was added Dess-Martin Periodinane (23.9 g, 56.4 mmol) at 0° C. for 5 min. Then the mixture was stirred at 25° C. for 2 hr. The mixture was concentrated and diluted with ethyl acetate (EA) (500 ml), washed with Na2S2O3 aq./NaHCO₃ aq. (500 ml/500 ml), brine (500 ml) and dried over Na2SO4. The organic layer was concentrated to give 2,3-bis (non-8-enoxy) propanal (11.7 g, crude, yield 90.1%) as a colorless oil.

1H NMR (500 MHZ, CDCl3) δ 9.72 (d, J=1.3 Hz, 1H), 5.81 (ddt, J=16.9, 10.2, 6.7 Hz, 2H), 5.05-4.87 (m, 4H), 3.86-3.33 (m, 7H), 2.09-1.97 (m, 4H), 1.61-1.20 (m, 20H).

### Synthesis of IY5613-E

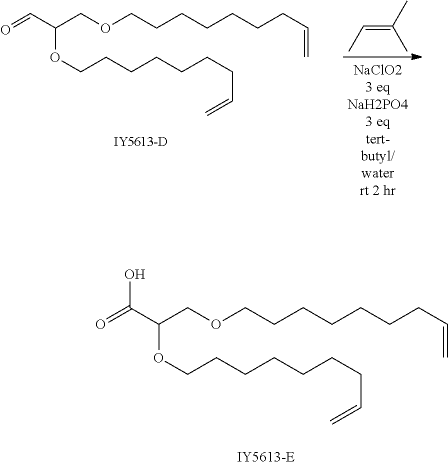

To a solution of 2,3-bis (non-8-enoxy) propanal (11.7 g, 34.6 mmol) in tert-butyl alcohol/water (180 ml/60 ml) was added sodium chlorite (9.38 g, 104 mmol, 2-Methyl-2-butene (60.6 g, 864 mmol) and sodium dihydrogen phosphate (9.38 g, 104 mmol). The mixture was stirred at 25° C. for 2 hr. Then the mixture was diluted with ethyl acetate (EA) (500 ml), washed with water (500 ml×2), brine (300 ml) and dried over Na2SO4. The organic layer was concentrated to give 2,3-bis (non-8-enoxy) propanoic acid (9.75 g, 27 mmol, yield 78%) as a colorless oil.

1H NMR (400 MHZ, CDCl3) δ 5.81 (ddt, J=16.9, 10.2, 6.7 Hz, 2H), 5.07-4.87 (m, 4H), 4.04 (dd, J=5.1, 3.3 Hz, 1H), 3.81-3.44 (m, 6H), 2.04 (dd, J=13.1, 6.5 Hz, 4H), 1.66-1.54 (m, 4H), 1.32 (ddd, J=12.7, 9.1, 5.4 Hz, 16H).

### Synthesis of IY5613-F

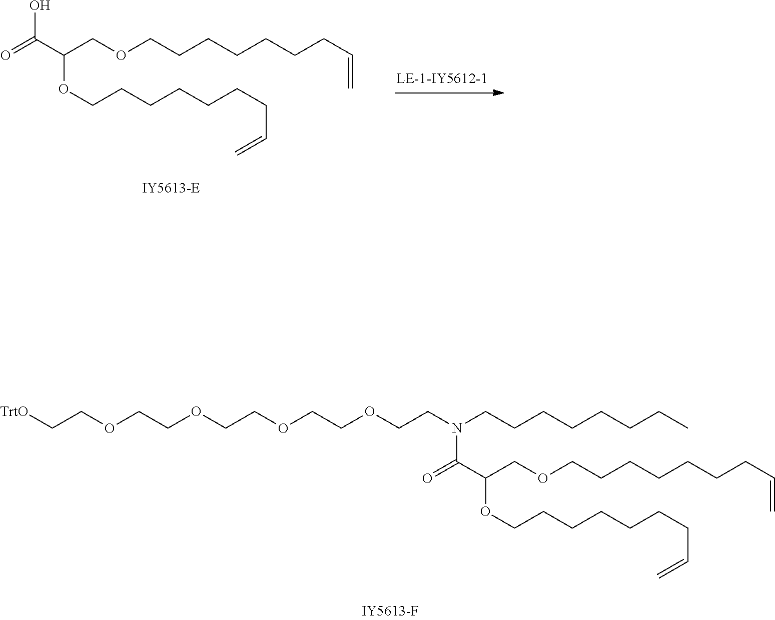

To a solution of N-[2-[2-[2-[2-(2-trityloxyethoxy) ethoxy]ethoxy] ethoxy]ethyl] octan-1-amine (9.52 g, 16.1 mmol) in DCM (150 ml) was added 2,3-bis (non-8-enoxy) propanoic acid (5 g, 14.1 mmol), [dimethylamino (triazolo[4,5-b]pyridin-3-yloxy) methylene]-dimethyl-ammonium;hexafluorophosphate (8.04 g, 21.2 mmol), N,N-diethylethanamine (2.85 g, 28.2 mmol). The mixture was stirred at 25° C. for 18 hr. Then the mixture was dealt with ethyl acetate (EA) (300 ml), washed with water (300 ml×2), brine (300 ml) and dried over Na2SO4. The organic layer was concentrated and purified by flash chromatography column (25% EA in PE) to give 2,3-bis (non-8-enoxy)-N-octyl-N-[2-[2-[2-[2-(2-trityloxyethoxy) ethoxy]ethoxy] ethoxy]ethyl]propanamide (8.22 g, 8.69 mmol, yield 61.5%) as a colorless oil.

1H NMR (500 MHZ, CDCl3) δ 7.46 (d, J=7.5 Hz, 6H), 7.29 (t, J=7.6 Hz, 6H), 7.22 (t, J=7.3 Hz, 3H), 5.86-5.74 (m, 2H), 5.03-4.89 (m, 4H), 4.43-4.29 (m, 1H), 3.70-3.22 (m, 29H), 2.03 (dd, J=13.5, 6.5 Hz, 4H), 1.57-1.51 (m, 4H), 1.40-1.23 (m, 28H), 0.88 (q, J=6.9 Hz, 3H).

### Synthesis of IY5613-G

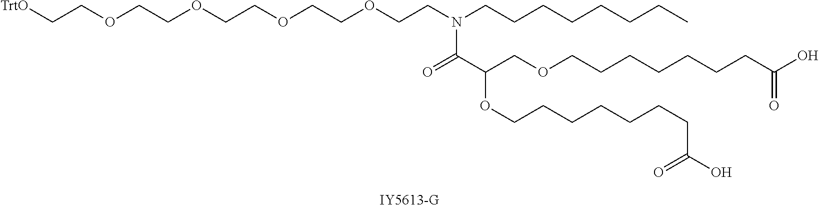

To a solution of 2,3-bis(oct-7-enoxy)-N-octyl-N-[2-[2-[2-[2-(2-trityloxyethoxy) ethoxy]ethoxy] ethoxy]ethyl]propanamide (8.22 g, 9.13 mmol) in ACN/CCl4/H₂O (80 ml/80 ml/80 ml) was added NalO4 (15.6 g, 73 mmol) ruthenium (III) chloride hydrate (412 mg, 1.83 mmol). The mixture was stirred at 25° C. for 18 hr. The mixture was filtered and dealt with ethyl acetate (EA) (500 ml), washed with Na₂S₂O₃ aq (300 ml), brine (300 ml) and dried over Na2SO4. The organic layer was concentrated and dealt with tert-butyl alcohol/water (120 ml/40 ml). sodium chlorite (2.48 g, 27.4 mmol), 2-Methyl-2-butene (16 g, 228 mmol) and sodium dihydrogen phosphate (3.29 g, 27.4 mmol) was added to the mixture. The mixture was stirred at 25° C. for 2 hr. Then the mixture was dealt with ethyl acetate (EA) (500 ml), washed with water (500 ml), brine (300 ml) and dried over Na2SO4. The organic was concentrated and purified by flash chromatography (10% MeOH in DCM) to give 7-[2-(6-carboxyhexoxy)-3-[octyl-[2-[2-[2-[2-(2-trityloxyethoxy) ethoxy]ethoxy] ethoxy]ethyl]amino]-3-oxo-propoxy] heptanoic acid (5.11 g, 5.19 mmol, yield 56.8%) as a grey oil.

1H NMR (400 MHZ, CDCl3) δ 7.48 (dd, J=15.2, 13.8 Hz, 6H), 7.29 (dd, J=10.1, 4.8 Hz, 6H), 7.22 (dd, J=8.3, 6.1 Hz, 3H), 4.38 (ddd, J=35.0, 7.3, 4.4 Hz, 1H), 3.80-3.33 (m, 26H), 3.23 (t, J=5.2 Hz, 2H), 2.50-2.24 (m, 4H), 1.56-1.18 (m, 28H), 0.87 (q, J=6.8 Hz, 3H).

### Synthesis of IY5613-7

To a solution of 7-[2-(6-carboxyhexoxy)-3-[octyl-[2-[2-[2-[2-(2-trityloxyethoxy) ethoxy]ethoxy] ethoxy]ethyl]amino]-3-oxo-propoxy] heptanoic acid (5.1 g, 5.45 mmol) in DCM (80 ml) was added (Z)-non-2-en-1-ol (1.86 mg, 13.1 mmol), EDC HCl (3.13 g, 16.3 mmol), DIEA (2.46 g, 19.1 mmol) and DMAP (333 mg). The mixture was stirred at 25° C. for 18 hr. Then the mixture was concentrated and purified by flash chromatography column (25% EA in PE) to give [(Z)-non-2-enyl] 8-[2-[8-[(Z)-non-2-enoxy]-8-oxo-octoxy]-3-[octyl-[2-[2-[2-[2-(2-trityloxyethoxy) ethoxy]ethoxy] ethoxy]ethyl]amino]-3-oxo-propoxyloctanoate (2.27 g, 1.83 mmol, yield 33.7%) as a colorless oil.

1H NMR (500 MHZ, CDCl3) δ 7.46 (d, J=7.4 Hz, 6H), 7.29 (t, J=7.5 Hz, 6H), 7.22 (t, J=7.3 Hz, 3H), 5.64 (dd, J=18.3, 7.5 Hz, 2H), 5.55-5.47 (m, 2H), 4.61 (d, J=6.9 Hz, 4H), 4.42-4.28 (m, 1H), 3.69-3.38 (m, 26H), 3.23 (t, J=5.2 Hz, 2H), 2.29 (t, J=7.5 Hz, 4H), 2.09 (q, J=7.3 Hz, 4H), 1.52-1.24 (m, 48H), 0.88 (t, J=6.8 Hz, 9H).

### Synthesis of IY5613-8

To a solution of [(Z)-non-2-enyl] 8-[2-[8-[(Z)-non-2-enoxy]-8-oxo-octoxy]-3-[octyl-[2-[2-[2-[2-(2-trityloxyethoxy) ethoxy]ethoxy] ethoxy]ethyl]amino]-3-oxo-propoxy] octanoate (2.38 g, 1.96 mmol) in THF/MeOH (15 ml/15 ml) was added Toluene-4-sulfonic acid (560 mg, 2.94 mmol). The mixture was stirred at 25° C. for 2 hr. Then the mixture was diluted with ethyl acetate (150 ml), washed with water (150 ml), brine (150 ml) and dried over Na2SO4. The mixture was concentrated and purified by flash chromatography column (5% MeOH in DCM) to give [(Z)-non-2-enyl] 8-[3-[2-[2-[2-[2-(2-hydroxyethoxy) ethoxy]ethoxy] ethoxy]ethyl-octyl-amino]-2-[8-[(Z)-non-2-enoxy]-8-oxo-octoxy]-3-oxo-propoxy] octanoate (1.51 g, 1.52 mmol, yield 77.7%) as a colorless oil.

1H NMR (400 MHZ, CDCl3) δ 5.69-5.46 (m, 4H), 4.62 (d, J=6.8 Hz, 4H), 4.43-4.29 (m, 1H), 3.74-3.40 (m, 28H), 2.29 (td, J=7.7, 1.5 Hz, 4H), 2.10 (dd, J=14.1, 7.0 Hz, 4H), 1.61-1.21 (m, 48H), 0.88 (td, J=6.7, 4.3 Hz, 9H).

### Synthesis of Compound XIII

To a solution of [(Z)-non-2-enyl] 8-[3-[2-[2-[2-[2-(2-hydroxyethoxy) ethoxy]ethoxy] ethoxy]ethyl-octyl-amino]-2-[8-[(Z)-non-2-enoxy]-8-oxo-octoxy]-3-oxo-propoxy] octanoate (400 mg, 0.41 mmol) in DCM (6 ml) was added 1-methylpiperidine-4-carboxylic acid (88.5 mg, 0.62 mmol), EDCl (158 mg, 0.82 mmol), DIEA (133 mg, 1.03 mmol) and DMAP (10 mg). The mixture was stirred at 25° C. for 18 h. Then the mixture was diluted with ethyl acetate (50 ml), washed with water (50 ml), brine (50 ml) and dried over Na2SO4. The organic was concentrated and purified by flash chromatography column (5% MeOH in DCM), to give 2-[2-[2-[2-[2-[2,3-bis [8-[(Z)-non-2-enoxy]-8-oxo-octoxy] propanoyl-octyl-amino]ethoxy]ethoxy] ethoxy]ethoxy]ethyl 1-methylpiperidine-4-carboxylate (269 mg, 0.24 mmol, yield 58.3%) as a colorless oil.

1H NMR (400 MHZ, CDCl3) δ 5.64 (dd, J=18.3, 7.5 Hz, 2H), 5.56-5.47 (m, 2H), 4.62 (d, J=6.9 Hz, 4H), 4.41-4.31 (m, 1H), 4.26-4.20 (m, 2H), 3.71-3.56 (m, 20H), 3.43 (ddd, J=18.7, 12.5, 7.2 Hz, 6H), 2.84 (s, 2H), 2.49-2.24 (m, 9H), 2.10 (dd, J=14.2, 6.8 Hz, 5H), 1.89 (d, J=44.2 Hz, 4H), 1.60 (d, J=6.8 Hz, 8H), 1.40-1.19 (m, 40H), 0.88 (dt, J=6.9, 4.3 Hz, 9H).

### Example 9: Synthesis of 2-[2-[2-[2-[2,3-bis [6-(2-octyldecanoyloxy) hexoxy] propoxy] ethoxy]ethoxy] ethoxy]ethyl 1-methylpiperidine-4-carboxylate (compound XIV)

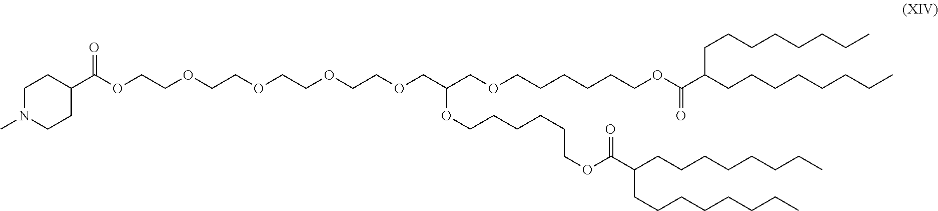 [SMILES: CCCCCCCCC(CCCCCCCC)C(=O)OCCCCCCOCC(COCCOCCOCCOCCOC(=O)C1CCN(C)CC1)OCCCCCCOC(=O)C(CCCCCCCC)CCCCCCCC]

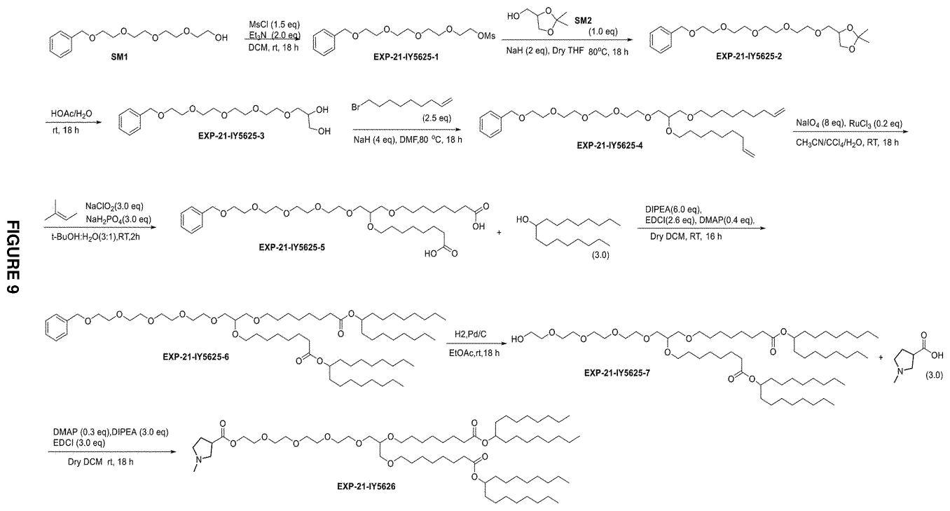

The compound XIV is prepared according to the schema of synthesis of FIG. 8.

### Synthesis of EXP-21-IJ5617

To a solution of 6-[3-[2-[2-[2-(2-benzyloxyethoxy) ethoxy]ethoxy] ethoxy]-2-(6-hydroxyhexoxy) propoxy] hexan-1-ol (1.6 g, 2.86 mmol) in dry DCM (20 mL) was added 2-octyldecanoic acid (2.44 g, 8.59 mmol), DIPEA (2.22 g, 17.2 mmol) and DMAP (0.14 g, 1.15 mmol) at room temperature. Then the mixture was cooled to 0° C. and portionwise over 15 min added EDCl (1.43 g, 7.45 mmol). The reaction was stirred for 17 hours at room temperature. TLC (5% CH3OH in DCM) shows that the reaction was finished. The reaction was poured into water and extracted with DCM. The water was extracted one more time with DCM. The combined organics were washed with brine, dried over Na2SO4 and concentrated under vacuum. Then purified by column chromatography with CH3OH in DCM (0-4%) (3%) to give 6-[3-[2-[2-[2-(2-benzyloxyethoxy) ethoxy]ethoxy] ethoxy]-2-[6-(2-octyldecanoyloxy) hexoxy] propoxy] hexyl 2-octyldecanoate (1.65 g, 52.8% yield) as light yellow oil.

1H NMR (400 MHZ, CDCl3) δ 7.37-7.27 (m, 5H), 4.57 (s, 2H), 4.10-4.01 (m, 4H), 3.71-3.38 (m, 25H), 2.35-2.26 (m, 2H), 1.67-1.34 (m, 24H), 1.33-1.17 (m, 50H), 0.87 (t, J=6.8 Hz, 12H).

A solution of 6-[3-[2-[2-[2-(2-benzyloxyethoxy) ethoxy]ethoxy] ethoxy]-2-[6-(2-octyldecanoyloxy) hexoxy] propoxy] hexyl 2-octyldecanoate (1.6 g, 1.47 mmol) in EtOAc (20 mL) was purged for 10 minutes with N2 followed by addition of Pd/C (20% wt/wt, 0.4 g) and the reaction continued purging with N2. The reaction was next evacuated under vacuum and backfilled with H2 3 times. The reaction was next stirred overnight at room temperature under an atmosphere of H2. TLC shows that the reaction was finished. The slurry filtered through celite and the celite was rinsed with EtOAc several times. The combined organics were next concentrated under vacuum to give 6-[3-[2-[2-[2-(2-hydroxyethoxy) ethoxy]ethoxy] ethoxy]-2-[6-(2-octyldecanoyloxy) hexoxy] propoxy] hexyl 2-octyldecanoate (1.38 g, 94.0% yield) as a light yellow oil.

1H NMR (400 MHZ, CDCl3) δ 4.15-4.02 (m, 4H), 3.75-3.40 (m, 25H), 2.35-2.26 (m, 2H), 1.68-1.53 (m, 12H), 1.46-1.34 (m, 12H), 1.27 (d, J=16.3 Hz, 48H), 0.88 (t, J=6.7 Hz, 12H).

### Synthesis of Compound XIV

To a solution of 6-[3-[2-[2-[2-(2-hydroxyethoxy) ethoxy]ethoxy] ethoxy]-2-[6-(2-octyldecanoyloxy) hexoxy] propoxy] hexyl 2-octyldecanoate (1.0 g, 1.00 mmol) in dry DCM (20 mL) was added 1-methylpiperidine-4-carboxylic acid (0.21 g, 1.50 mmol), DIPEA (0.39 g, 3.00 mmol) and DMAP (0.02 g, 0.20 mmol) at room temperature. Then the mixture was cooled to 0° C. and portion wise over 15 min added EDCl (0.29 g, 1.50 mmol). The reaction was stirred for 17 hours at room temperature. TLC shows that the reaction was finished. The reaction was poured into water and extracted with DCM. The water was extracted one more time with DCM. The combined organics phases were washed with brine, dried over Na2SO4 and concentrated under vacuum. Then purified by column chromatography with CH3OH in DCM (0-10%) (8%) to give 2-[2-[2-[2-[2,3-bis [6-(2-octyldecanoyloxy) hexoxy] propoxy] ethoxy]ethoxy] ethoxy]ethyl 1-methylpiperidine-4-carboxylate (476 mg, 42.3% yield) as light yellow oil.

1H NMR (400 MHZ, CDCl3) δ 4.27-4.20 (m, 2H), 4.10-4.02 (m, 4H), 3.71-3.39 (m, 23H), 2.82 (d, J=11.6 Hz, 2H), 2.32-2.26 (m, 5H), 2.13 (s, 2H), 2.02-1.90 (m, 3H), 1.84-1.73 (m, 2H), 1.68-1.52 (m, 12H), 1.49-1.34 (m, 12H), 1.32-1.21 (m, 48H), 0.88 (t, J=6.8 Hz, 12H).

### Example 10: Synthesis of 2-[2-[2-[2-[2,3-bis [8-(1-octylnonoxy)-8-oxo-octoxy] propoxy] ethoxy]ethoxy] ethoxy]ethyl 1-methylpyrrolidine-3-carboxylate (compound XV)

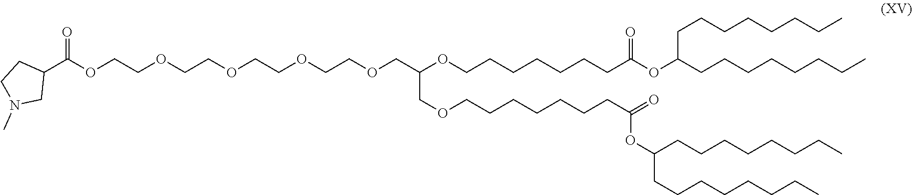 [SMILES: CCCCCCCCC(CCCCCCCC)OC(=O)CCCCCCCOCC(COCCOCCOCCOCCOC(=O)C1CCN(C)C1)OCCCCCCCC(=O)OC(CCCCCCCC)CCCCCCCC]

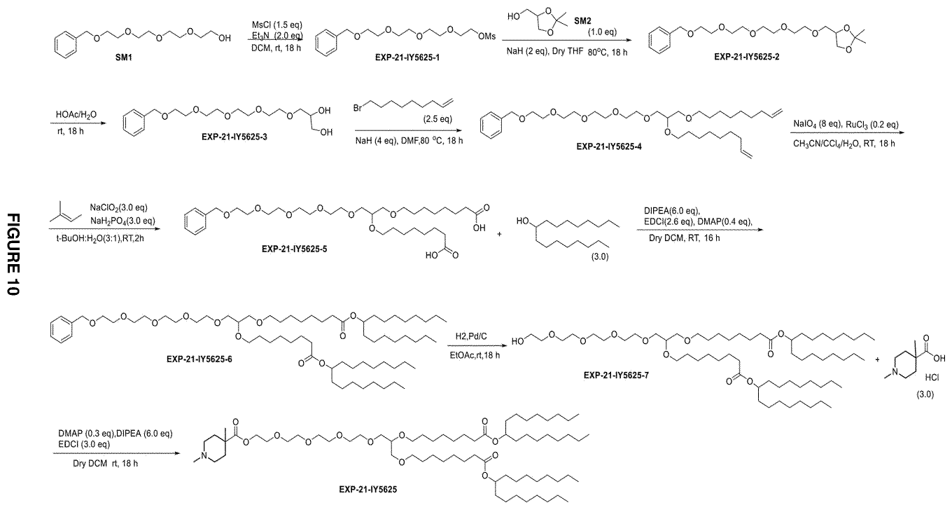

The compound XV is prepared according to the schema of synthesis of FIG. 9.

### Synthesis of the Following Compound

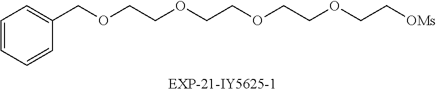 [SMILES: CS(=O)(=O)OCCOCCOCCOCCOCc1ccccc1]

To a mixture of 2-[2-[2-(2-benzyloxyethoxy) ethoxy]ethoxy] ethanol (50 g, 176 mmol) and Et₃N (35.6 g, 352 mmol) in DCM (400 mL) was added methanesulfonyl chloride (30.2 g, 264 mmol) slowly at 0° C. The mixture was stirred overnight at room temperature. CH₂Cl₂ (400 mL) were added to the solution, and the mixture was washed with diluted HCl (1M, 1000 mL). The mixture was shaken, the layers were separated, and the organic layer was collected. The organic layer was further washed with Water (1000 mL) and brine (1000 mL), and dried over Na2SO4. Solvent was then removed to give 2-[2-[2-(2-benzyloxyethoxy) ethoxy]ethoxy]ethyl methanesulfonate (64.1 g, 172 mmol, 97.5% yield) as an orange oil.

1H NMR (400 MHZ, CDCl3) δ 7.37-7.27 (m, 5H), 4.56 (s, 2H), 4.40-4.33 (m, 2H), 3.78-3.72 (m, 2H), 3.69-3.60 (m, 12H), 3.06 (s, 3H).

### Synthesis of EXP-21-IY5625-2

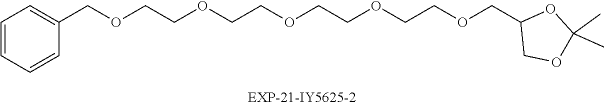 [SMILES: CC1(C)OCC(COCCOCCOCCOCCOCc2ccccc2)O1]

To the solution of (2,2-dimethyl-1,3-dioxolan-4-yl) methanol (24.6 g, 177 mmol) in THF (500 mL) was added NaH (14.1 g, 354 mmol) and the mixture was heated to 80° C. for 30 min. Then the reaction was cooled to room temperature and 2-[2-[2-(2-benzyloxyethoxy) ethoxy]ethoxy]ethyl methanesulfonate (64.1 g, 177 mmol) was added under nitrogen and the reaction was heated at 80° C. for 24 h. TLC indicated that the starting material was consumed. The reaction was quenched with water (300 mL) and extracted with ethyl acetate (600 mL). The aqueous layer was extracted with ethyl acetate (EA) (600 mL) again. The combined organic layers were washed with brine, dried over sodium sulfate, filtered and concentrated. The residue was purified through flash chromatography eluted with 20 to 50% ethyl acetate in petroleum ether to give 24-[2-[2-[2-(2-benzyloxyethoxy) ethoxy]ethoxy] ethoxymethyl]-2,2-dimethyl-1,3-dioxolane (48.635 g, 116 mmol, 65.6% yield) as light yellow oil.

LCMS; Find peak MS (ESI) m/z=421.4 (M±23)⁺ at 2.154 1H NMR (500 MHz, CDCl3) δ 7.36-7.26 (m, 5H), 4.57 (s, 2H), 4.32-4.22 (m, 1H), 4.07-4.01 (m, 1H), 3.75-3.70 (m, 1H), 3.69-3.60 (m, 16H), 3.59-3.55 (m, 1H), 3.51-3.47 (m, 1H), 1.42 (s, 3H), 1.35 (s, 3H).

### Synthesis of EXP-21-IY5625-3

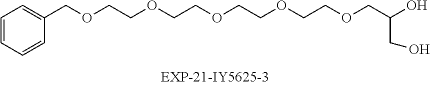 [SMILES: OCC(O)COCCOCCOCCOCCOCc1ccccc1]

The mixture of 4-[2-[2-[2-(2-benzyloxyethoxy) ethoxy]ethoxy] ethoxymethyl]-2,2-dimethyl-1,3-dioxolane (48.635 g, 122 mmol) in AcOH (200 mL) and H2O (200 mL) was stirred at room temperature for 18 h. TLC (EA/PE 1/1, SM Rf: 0.5; product, Rf: 0.1) indicated that all the starting materials was consumed. The solvent was removed under vacuum and azeotroped with toluene several times. 2-[2-[2-(2-methylsulfonyloxyethoxy) ethoxy]ethoxy]ethyl methanesulfonate (43.7 g, 116 mmol, quant.) as light yellow oil was obtained which was used without further purification.

1H NMR (400 MHZ, CDCl3) δ 7.36-7.23 (m, 5H), 4.56 (s, 2H), 3.89-3.80 (m, 1H), 3.72-3.49 (m, 21H).

### Synthesis of EXP-21-IY5625-4

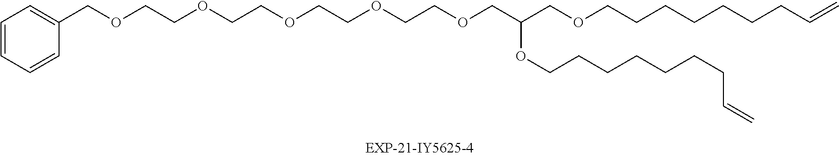 [SMILES: C=CCCCCCCCOCC(COCCOCCOCCOCCOCc1ccccc1)OCCCCCCCC=C]

To a solution of 3-[2-[2-[2-(2-benzyloxyethoxy) ethoxy]ethoxy] ethoxy]propane-1,2-diol (24 g, 60.3 mmol) in dry DMF (200 mL) under nitrogen was added NaH (9.64 g, 241 mmol) and the mixture was heated at 80° C. for 15 min. Then the reaction was cooled to room temperature and 9-bromonon-1-ene (31.9 g, 151 mmol) was added dropwise to this solution. The mixture was stirred at room temperature for 30 min and then at 80° C. for 18 h. TLC (EA/PE=1/1, Rf: 0.5) indicated that a new spot was formed. The reaction was quenched with water (50 mL) and then partitioned between ethyl acetate and water. The aqueous layer was extracted with ethyl acetate again. The combined organic layers were dried over sodium sulfate, filtered and concentrated. The residue was purified by flash chromatography eluted with 20% to 50% ethyl acetate ir petroleum ether to give 2-[2-[2-[2-[2,3-bis (non-8-enoxy) propoxy] ethoxy]ethoxy] ethoxy]ethoxymethylbenzene (13.24 g, 20.7 mmol, 32.6% yield) as light yellow oil.

1H NMR (500 MHZ, CDCl3) δ 7.36-7.27 (m, 5H), 5.86-5.75 (m, 2H), 5.03-4.89 (m, 4H), 4.57 (s, 2H), 3.69-3.61 (m, 17H), 3.58-3.40 (m, 9H), 2.08-1.99 (m, 4H), 1.61-1.51 (m, 4H), 1.42-1.27 (m, 16H).

### Synthesis of EXP-21-IY5625-5

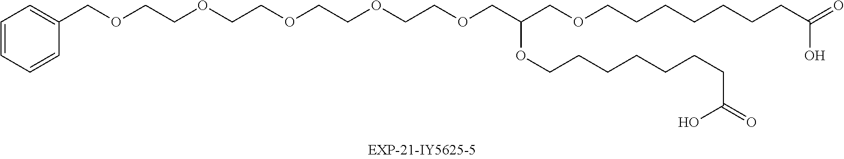 [SMILES: O=C(O)CCCCCCCOCC(COCCOCCOCCOCCOCc1ccccc1)OCCCCCCCC(=O)O]

To a solution of 2-[2-[2-[2-[2,3-bis (non-8-enoxy) propoxy] ethoxy]ethoxy] ethoxy]ethoxymethyl benzene (13.24 g, 20.7 mmol) in MeCN (120 mL), CCl4 (120 mL) and water (120 mL) was added NalO4 (35.5 g, 166 mmol) and RuCl3 (935 mg, 4.15 mmol). The reaction mixture was stirred at room temperature for 18 h. LCMS indicated that the title compound was the major product along with partial mono-aldehyde product. The reaction was filtered and the filtrate was diluted with ethyl acetate (800 mL) and washed with 1N aq. HCl (400 mL). The organic layer was washed with Na2S2O3 solution and then dried over sodium sulfate, filtered and concentrated to give 8-[3-[2-[2-[2-(2-benzyloxyethoxy) ethoxy]ethoxy] ethoxy]-2-(7-carboxyheptoxy) propoxy] octanoic acid (13.3 g, 8.28 mmol, 39.9% yield) as pale green oil which was used without further purification.

1H NMR (500 MHZ, CDCl3) δ 9.79-9.72 (m, 1H), 7.36-7.27 (m, 5H), 4.57 (s, 2H), 3.72-3.37 (m, 26H), 2.45-2.39 (m, 1H), 2.32 (t, J=7.3 Hz, 2H), 1.68-1.49 (m, 8H), 1.32 (s, 12H).

8-[3-[2-[2-[2-(2-benzyloxyethoxy) ethoxy]ethoxy] ethoxy]-2-(8-oxooctoxy) propoxy] octanoic acid (13.3 g, 12.7 mmol, 60% purity) was dissolved in t-BuOH: H₂O (3:1, 100 mL), containing NaH₂PO₄.2H₂O (7.64 g, 63.7 mmol), 2-methy-2-butene (60 mL) and sodium chlorite (7.2 g, 63.7 mmol). The reaction was stirred for 2 h at room temperature and LCMS indicated that the starting material was consumed. The reaction mixture was diluted with ethyl acetate. The aqueous layer was extracted with ethyl acetate.

The combined extract was dried over sodium sulfate. The residue was purified by flash chromatography eluted with 0% to 5% CH₃OH in DCM to give 8-[3-[2-[2-[2-(2-benzyloxyethoxy) ethoxy]ethoxy] ethoxy]-2-(7-carboxyheptoxy) propoxy] octanoic acid (6.65 g, 9.83 mmol, 77.2% yield) and partial oxidation product (2.78 g) as light yellow oil.

1H NMR (500 MHZ, CDCl3) δ 7.37-7.27 (m, 5H), 4.57 (s, 2H), 3.69-3.61 (m, 16H), 3.58-3.40 (m, 10H), 2.38-2.25 (m, 4H), 1.68-1.48 (m, 8H), 1.34 (d, J=14.9 Hz, 12H).

### Synthesis of EXP-21-IY5625-6

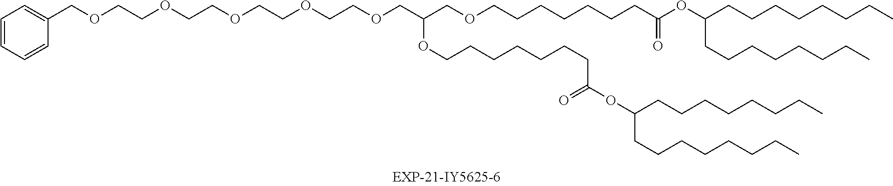 [SMILES: CCCCCCCCC(CCCCCCCC)OC(=O)CCCCCCCOCC(COCCOCCOCCOCCOCc1ccccc1)OCCCCCCCC(=O)OC(CCCCCCCC)CCCCCCCC]

A mixture of 8-[3-[2-[2-[2-(2-benzyloxyethoxy) ethoxy]ethoxy] ethoxy]-2-(7-carboxyheptoxy) propoxy] octanoic acid (6.65 g, 10.3 mmol), heptadecan-9-ol (7.96 g, 31 mmol), DMAP (506 mg, 4.14 mmol), EDCl HCl (5.16 g, 26.9 mmol) and DIEA (8.02 g, 62.1 mmol) in Dry DCM (100 mL). The mixture was stirred for 16 h at room temperature. The mixture was added DCM (500 mL) and washed with 1 N HCl, NaCl and concentrated. The residue was purified by flash column chromatography on silica gel eluting with CH₃OH in DCM (0 to 4%) to give 1-octylnonyl 8-[3-[2-[2-[2-(2-benzyloxyethoxy) ethoxy]ethoxy] ethoxy]-2-[8-(1-octylnonoxy)-8-oxo-octoxy] propoxy] octanoate (3.6 g, 3.05 mmol, 30.5% yield) as pale yellow oil. 1H NMR (400 MHZ, CDCl3) δ 7.36-7.27 (m, 5H), 4.90-4.81 (m, 2H), 4.57 (s, 2H), 3.71-3.61 (m, 16H), 3.59-3.39 (m, 9H), 2.27 (t, J=7.1 Hz, 4H), 1.62-1.46 (m, 16H), 1.28 (d, J=22.7 Hz, 60H), 0.91-0.84 (m, 12H).

### Synthesis of EXP-21-IY5625-7

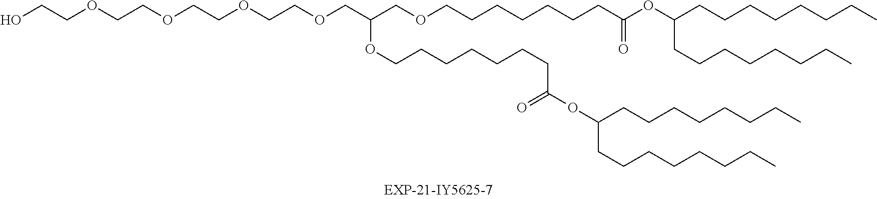 [SMILES: CCCCCCCCC(CCCCCCCC)OC(=O)CCCCCCCOCC(COCCOCCOCCOCCO)OCCCCCCCC(=O)OC(CCCCCCCC)CCCCCCCC]

To the solution of 1-octylnonyl 8-[3-[2-[2-[2-(2-benzyloxyethoxy) ethoxy]ethoxy] ethoxy]-2-[8-(1-octylnonoxy)-8-oxo-octoxy] propoxy] octanoate (3.6 g, 3.22 mmol) in ethyl acetate (50 mL) was added Pd/C (1.1 g, 30% wt/wt). The mixture was stirred at room temperature under hydrogen for 18 h. TLC (CH₃OH/DCM (3%)) indicated that the starting material was consumed. The reaction was filtered through celite and washed with ethyl acetate to give1-octylnonyl 8-[3-[2-[2-[2-(2-hydroxy ethoxy) ethoxy]ethoxy] ethoxy]-2-[8-(1-octylnonoxy)-8-oxo-octoxy] propoxyloctanoate (3.187 g, 3.1 mmol, 96.3% yield) as colorless oil. 1H NMR (400 MHZ, CDCl3) δ 4.91-4.80 (m, 2H), 3.75-3.71 (m, 2H), 3.70-3.39 (m, 23H), 2.27 (t, J=7.5 Hz, 4H), 1.67-1.45 (m, 16H), 1.35-1.20 (m, 60H), 0.92-0.81 (m, 12H).

### Synthesis of Compound XV

To a solution of 1-octylnonyl 8-[3-[2-[2-[2-(2-hydroxyethoxy) ethoxy]ethoxy] ethoxy]-2-[8-(1-octylnonoxy)-8-oxo-octoxy] propoxy] octanoate (0.500 g, 0.486 mmol) and 1-methylpyrrolidine-3-carboxylic acid (0.188 g, 1.46 mmol) in dry dichloromethane (15 mL) was added DIPEA (0.188 g, 1.46 mmol) and DMAP (0.018 g, 0.146 mmol). Then added to EDCl (0.279 g, 1.46 mmol) at 0° C. The reaction was allowed to stir at room temperature for 18 h. The reaction was diluted with dichloromethane and washed with saturated sodium bicarbonate. The organic layer was separated and washed with brine, and dried over Na2SO4. The organic layer was filtered and evaporated under vacuum. The residue was purified by silica gel chromatography (0-5% CH₃OH in DCM (4%))_to give 2-[2-[2-[2-[2,3-bis [8-(1-octylnonoxy)-8-oxo-octoxy] propoxy] ethoxy]ethoxy] ethoxy]ethyl 1-methylpyrrolidine-3-carboxylate (0.3149 g, 0.271 mmol, 55.7% yield).

LCMS; Find peak MS (ESI) m/z=1141.9 (M+H)⁺ at 2.54

1H NMR (400 MHZ, CDCl3) δ 4.92-4.80 (m, 2H), 4.29-4.22 (m, 2H), 3.75-3.37 (m, 23H), 3.18-3.06 (m, 1H), 2.92 (s, 1H), 2.78-2.51 (m, 3H), 2.41 (s, 3H), 2.30-2.24 (m, 4H), 2.20-2.09 (m, 2H), 1.64-1.45 (m, 16H), 1.35-1.20 (m, 60H), 0.93-0.83 (m, 12H).

### Example 11: Synthesis of 2-[2-[2-[2-[2,3-bis [8-(1-octylnonoxy)-8-oxo-octoxy] propoxy] ethoxy]ethoxy] ethoxy]ethyl 1-methylpiperidine-3-carboxylate (compound XVI)

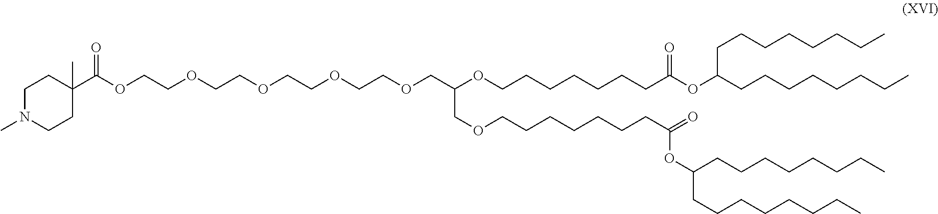 [SMILES: CCCCCCCCC(CCCCCCCC)OC(=O)CCCCCCCOCC(COCCOCCOCCOCCOC(=O)C1(C)CCN(C)CC1)OCCCCCCCC(=O)OC(CCCCCCCC)CCCCCCCC]

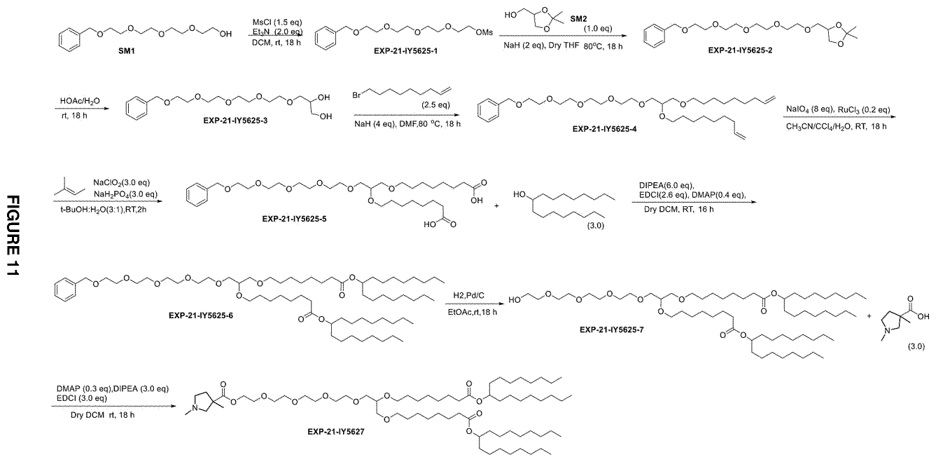

The compound XVI is prepared according to the schema of synthesis of FIG. 10.

### Synthesis of EXP-21-IY5625-1

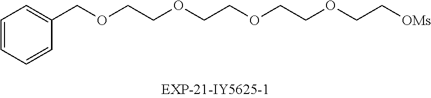 [SMILES: CS(=O)(=O)OCCOCCOCCOCCOCc1ccccc1]

To a mixture of 2-[2-[2-(2-benzyloxyethoxy) ethoxy]ethoxy] ethanol (50 g, 176 mmol) and Et₃N (35.6 g, 352 mmol) in DCM (400 mL) was added methanesulfonyl chloride (30.2 g, 264 mmol) slowly at 0° C. The mixture was stirred overnight at room temperature. CH₂Cl₂ (400 mL) were added to the solution, and the mixture was washed with diluted HCl (1M, 1000 mL). The mixture was shaken, the layers were separated, and the organic layer was collected. The organic layer was further washed with water (1000 mL) and brine (1000 mL), and dried over Na2SO4. Solvent was then removed to give 2-[2-[2-(2-benzyloxyethoxy) ethoxy]ethoxy]ethyl methanesulfonate (64.1 g, 172 mmol, 97.5% yield) as an orange oil.

1H NMR (400 MHZ, CDCl3) δ 7.37-7.27 (m, 5H), 4.56 (s, 2H), 4.40-4.33 (m, 2H), 3.78-3.72 (m, 2H), 3.69-3.60 (m, 12H), 3.06 (s, 3H)

### Synthesis of EXP-21-IY5625-2

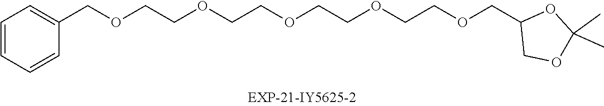 [SMILES: CC1(C)OCC(COCCOCCOCCOCCOCc2ccccc2)O1]

To the solution of (2,2-dimethyl-1,3-dioxolan-4-yl) methanol (24.6 g, 177 mmol) in THF (500 mL) was added NaH (14.1 g, 354 mmol) and the mixture was heated to 80° C. for 30 min. Then the reaction was cooled to room temperature and 2-[2-[2-(2-benzyloxyethoxy) ethoxy]ethoxy]ethyl methane

sulfonate (64.1 g, 177 mmol) was added under nitrogen and the reaction was heated at 80° C. for 24 h. TLC indicated that the starting material was consumed. The reaction was quenched with water (300 mL) and extracted with ethyl acetate (600 mL). The aqueous layer was extracted with ethyl acetate (EA) (600 mL) again. The combined organic layers were washed with brine, dried over sodium sulfate, filtered and concentrated. The residue was purified through flash chromatography eluted with 20 to 50% ethyl acetate in petroleum ether to give 24-[2-[2-[2-(2-benzyloxyethoxy) ethoxy]ethoxy] ethoxymethyl]-2,2-dimethyl-1,3-dioxolane (48.635 g, 116 mmol, 65.6% yield) as light yellow oil.

-LCMS; Find peak MS (ESI) m/z=421.4 (M±23)⁺ at 2.154

¹H NMR (500 MHZ, CDCl3) δ 7.36-7.26 (m, 5H), 4.57 (s, 2H), 4.32-4.22 (m, 1H), 4.07-4.01 (m, 1H), 3.75-3.70 (m, 1H), 3.69-3.60 (m, 16H), 3.59-3.55 (m, 1H), 3.51-3.47 (m, 1H), 1.42 (s, 3H), 1.35 (s, 3H).

### Synthesis of EXP-21-IY5625-3

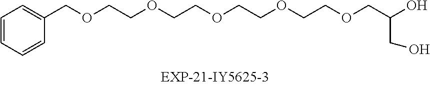 [SMILES: OCC(O)COCCOCCOCCOCCOCc1ccccc1]

The mixture of 4-[2-[2-[2-(2-benzyloxyethoxy) ethoxy]ethoxy] ethoxymethyl]-2,2-dimethyl-1,3-dioxolane (48.635 g, 122 mmol) in AcOH (200 mL) and H2O (200 mL) was stirred at room temperature for 18 h. TLC (EA/PE 1/1, SM Rf: 0.5; product, Rf: 0.1) indicated that all the starting materials were consumed. The solvent was removed under vacuum and azeotroped with toluene several times. 2-[2-[2-(2-methylsulfonyloxyethoxy) ethoxy]ethoxy]ethyl methanesulfonate (43.7 g, 116 mmol, quant.) as light yellow oil was obtained which was used without further purification.

1H NMR (400 MHZ, CDCl3) δ 7.36-7.23 (m, 5H), 4.56 (s, 2H), 3.89-3.80 (m, 1H), 3.72-3.49 (m, 21H).

### Synthesis of EXP-21-IY5625-4

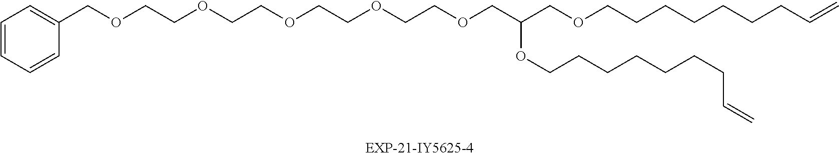 [SMILES: C=CCCCCCCCOCC(COCCOCCOCCOCCOCc1ccccc1)OCCCCCCCC=C]

To a solution of 3-[2-[2-[2-(2-benzyloxyethoxy) ethoxy]ethoxy] ethoxy]propane-1,2-diol (24 g, 60.3 mmol) in dry DMF (200 mL) under nitrogen was added NaH (9.64 g, 241 mmol) and the mixture was heated at 80° C. for 15 min. Then the reaction was cooled to room temperature and 9-bromonon-1-ene (31.9 g, 151 mmol) was added dropwise to this solution. The mixture was stirred at room temperature for 30 min and then at 80° C. for 18 h. TLC (EA/PE=1/1, Rf: 0.5) indicated that a new spot was formed. The reaction was quenched with water (50 mL) and then partitioned between ethyl acetate and water. The aqueous layer was extracted with ethyl acetate again. The combined organic layers were dried over sodium sulfate, filtered and concentrated. The residue was purified by flash chromatography eluted with 20% to 50% ethyl acetate in petroleum ether to give 2-[2-[2-[2-[2,3-bis (non-8-enoxy) propoxy] ethoxy]ethoxy] ethoxy]ethoxymethylbenzene (13.24 g, 20.7 mmol, 32.6% yield) as light yellow oil.

1H NMR (500 MHZ, CDCl3) δ 7.36-7.27 (m, 5H), 5.86-5.75 (m, 2H), 5.03-4.89 (m, 4H), 4.57 (s, 2H), 3.69-3.61 (m, 17H), 3.58-3.40 (m, 9H), 2.08-1.99 (m, 4H), 1.61-1.51 (m, 4H), 1.42-1.27 (m, 16H).

### Synthesis of EXP-21-IY5625-5

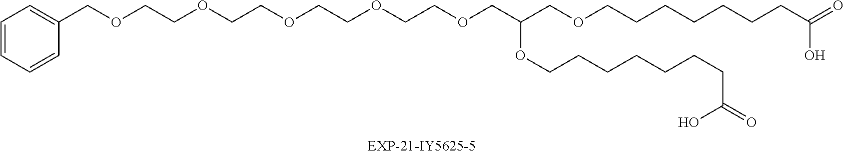 [SMILES: O=C(O)CCCCCCCOCC(COCCOCCOCCOCCOCc1ccccc1)OCCCCCCCC(=O)O]

To a solution of 2-[2-[2-[2-[2,3-bis (non-8-enoxy) propoxy] ethoxy]ethoxy] ethoxy]ethoxymethyl benzene (13.24 g, 20.7 mmol) in MeCN (120 mL), CCl4 (120 mL) and water (120 mL) was added NalO4 (35.5 g, 166 mmol) and RuCl3 (935 mg, 4.15 mmol). The reaction mixture was stirred at room temperature for 18 h. LCMS indicated that the title compound was the major product along with partial mono-aldehyde product. The reaction was filtered, and the filtrate was diluted with ethyl acetate (800 mL) and washed with 1N aq. HCl (400 mL). The organic layer was washed with Na2S₂O₃ solution and then dried over sodium sulfate, filtered and concentrated to give 8-[3-[2-[2-[2-(2-benzyloxyethoxy) ethoxy]ethoxy] ethoxy]-2-(7-carboxyheptoxy) propoxy] octanoic acid (13.3 g, 8.28 mmol, 39.9% yield) as pale green oil which was used without further purification.

1H NMR (500 MHZ, CDCl3) δ 9.79-9.72 (m, 1H), 7.36-7.27 (m, 5H), 4.57 (s, 2H), 3.72-3.37 (m, 26H), 2.45-2.39 (m, 1H), 2.32 (t, J=7.3 Hz, 2H), 1.68-1.49 (m, 8H), 1.32 (s, 12H).

8-[3-[2-[2-[2-(2-benzyloxyethoxy) ethoxy]ethoxy] ethoxy]-2-(8-oxooctoxy) propoxy] octanoic acid (13.3 g, 12.7 mmol, 60% purity) was dissolved in t-BuOH: H2O (3:1, 100 mL), containing NaH₂PO₄.2H₂O (7.64 g, 63.7 mmol), 2-methy-2-butene (60 mL) and sodium chlorite (7.2 g, 63.7 mmol). The reaction was stirred for 2 h at room temperature and LCMS indicated that the starting material was consumed. The reaction mixture was diluted with ethyl acetate. The aqueous layer was extracted with ethyl acetate. The combined extract was dried over sodium sulfate. The residue was purified by flash chromatography eluted with 0% to 5% methanol in DCM to give 8-[3-[2-[2-[2-(2-benzyloxyethoxy) ethoxy]ethoxy] ethoxy]-2-(7-carboxyheptoxy) propoxy] octanoic acid (6.65 g, 9.83 mmol, 77.2% yield) and partial oxidation product (2.78 g) as light yellow oil.

1H NMR (500 MHZ, CDCl3) δ 7.37-7.27 (m, 5H), 4.57 (s, 2H), 3.69-3.61 (m, 16H), 3.58-3.40 (m, 10H), 2.38-2.25 (m, 4H), 1.68-1.48 (m, 8H), 1.34 (d, J=14.9 Hz, 12H).

### Synthesis of EXP-21-IY5625-6

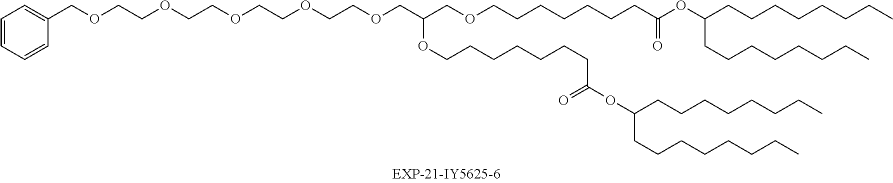 [SMILES: CCCCCCCCC(CCCCCCCC)OC(=O)CCCCCCCOCC(COCCOCCOCCOCCOCc1ccccc1)OCCCCCCCC(=O)OC(CCCCCCCC)CCCCCCCC]

A mixture of 8-[3-[2-[2-[2-(2-benzyloxyethoxy) ethoxy]ethoxy] ethoxy]-2-(7-carboxyheptoxy) propoxy] octanoic acid (6.65 g, 10.3 mmol), heptadecan-9-ol (7.96 g, 31 mmol), DMAP (506 mg, 4.14 mmol), EDCl HCl (5.16 g, 26.9 mmol) and Diisopropylethylamine (DIEA) (8.02 g, 62.1 mmol) in Dry DCM (100 mL). The mixture was stirred for 16 h at room temperature. The mixture was added DCM (500 mL) and washed with 1 N HCl, NaCl and concentrated. The residue was purified by flash column chromatography on silica gel eluting with CH₃OH in DCM (0 to 4%) to give 1-octylnonyl 8-[3-[2-[2-[2-(2-benzyloxyethoxy) ethoxy]ethoxy] ethoxy]-2-[8-(1-octylnonoxy)-8-oxo-octoxy] propoxy] octanoate (3.6 g, 3.05 mmol, 30.5% yield) as pale yellow oil.

1H NMR (400 MHZ, CDCl3) 0 7.36-7.27 (m, 5H), 4.90-4.81 (m, 2H), 4.57 (s, 2H), 3.71-3.61 (m, 16H), 3.59-3.39 (m, 9H), 2.27 (t, J=7.1 Hz, 4H), 1.62-1.46 (m, 16H), 1.28 (d, J=22.7 Hz, 60H), 0.91-0.84 (m, 12H).

### Synthesis of EXP-21-IY5625-7

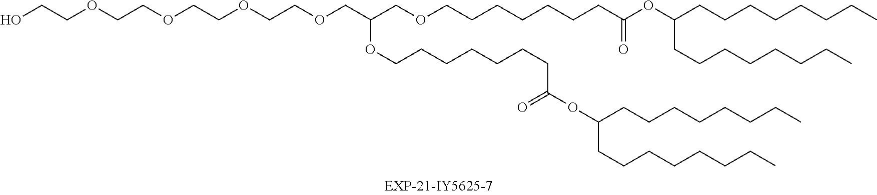 [SMILES: CCCCCCCCC(CCCCCCCC)OC(=O)CCCCCCCOCC(COCCOCCOCCOCCO)OCCCCCCCC(=O)OC(CCCCCCCC)CCCCCCCC]

To the solution of 1-octylnonyl 8-[3-[2-[2-[2-(2-benzyloxyethoxy) ethoxy]ethoxy] ethoxy]-2-[8-(1-octylnonoxy)-8-oxo-octoxy] propoxy] octanoate (3.6 g, 3.22 mmol) in ethyl acetate (50 mL) was added Pd/C (1.1 g, 30% wt/wt). The mixture was stirred at room temperature under hydrogen for 18 h. TLC (CH₃OH/DCM (3%)) indicated that the starting material was consumed. The reaction was filtered through celite and washed with ethyl acetate to give1-octylnonyl 8-[3-[2-[2-[2-(2-hydroxy ethoxy) ethoxy]ethoxy] ethoxy]-2-[8-(1-octylnonoxy)-8-oxo-octoxy] propoxyloctanoate (3.187 g, 3.1 mmol, 96.3% yield) as colorless oil. 1H NMR (400 MHZ, CDCl3) δ 4.91-4.80 (m, 2H), 3.75-3.71 (m, 2H), 3.70-3.39 (m, 23H), 2.27 (t, J=7.5 Hz, 4H), 1.67-1.45 (m, 16H), 1.35-1.20 (m, 60H), 0.92-0.81 (m, 12H).

### Synthesis of Compound XVI

To a solution of 1-octylnonyl 8-[3-[2-[2-[2-(2-hydroxyethoxy) ethoxy]ethoxy] ethoxy]-2-[8-(1-octylnonoxy)-8-oxo-octoxy] propoxyloctanoate (0.500 g, 0.486 mmol) and 1-methylpyrrolidine-3-carboxylic acid (0.297 g, 1.46 mmol) in dry dichloromethane (15 mL) was added DIPEA (0.377 g, 2.97 mmol) and DMAP (0.018 g, 0.146 mmol). Then added to EDCl (0.279 g, 1.46 mmol) at 0° C. The reaction was allowed to stir at room temperature for 18 h. The reaction was diluted with dichloromethane and washed with saturated sodium bicarbonate. The organic layer was separated and washed with brine and dried over Na2SO4. The organic layer was filtered and evaporated under vacuum. The residue was purified by silica gel chromatography (0-5% CH₃OH in DCM (4%)) to give 2-[2-[2-[2-[2,3-bis [8-(1-octylnonoxy)-8-oxo-octoxy] propoxy] ethoxy]ethoxy] ethoxy]ethyl1,4-dimethylpiperidine-4-carboxylate (0.4171 g, 0.350 mmol, 72.0% yield) as pale yellow oil.

LCMS; Find peak MS (ESI) m/z=1169.1 (M+H)⁺ at 2.3 min

1H NMR (400 MHZ, CDCl3) δ 4.91-4.81 (m, 2H), 4.31-4.25 (m, 2H), 3.72-3.39 (m, 23H), 2.78 (s, 2H), 2.36 (s, 3H), 2.32-2.14 (m, 8H), 1.64-1.47 (m, 16H), 1.34-1.20 (m, 65H), 0.92-0.84 (m, 12H).

### Example 12: Synthesis of 2-[2-[2-[2-[2,3-bis [8-(1-octylnonoxy)-8-oxo-octoxy] propoxy] ethoxy]ethoxy] ethoxy]ethyl 1,3-dimethylpyrrolidine-3-carboxylate (compound XVII)

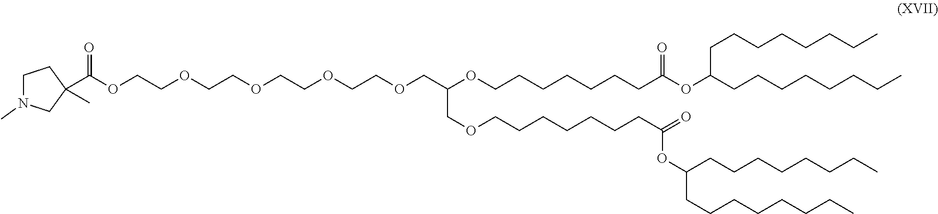 [SMILES: CCCCCCCCC(CCCCCCCC)OC(=O)CCCCCCCOCC(COCCOCCOCCOCCOC(=O)C1(C)CCN(C)C1)OCCCCCCCC(=O)OC(CCCCCCCC)CCCCCCCC]

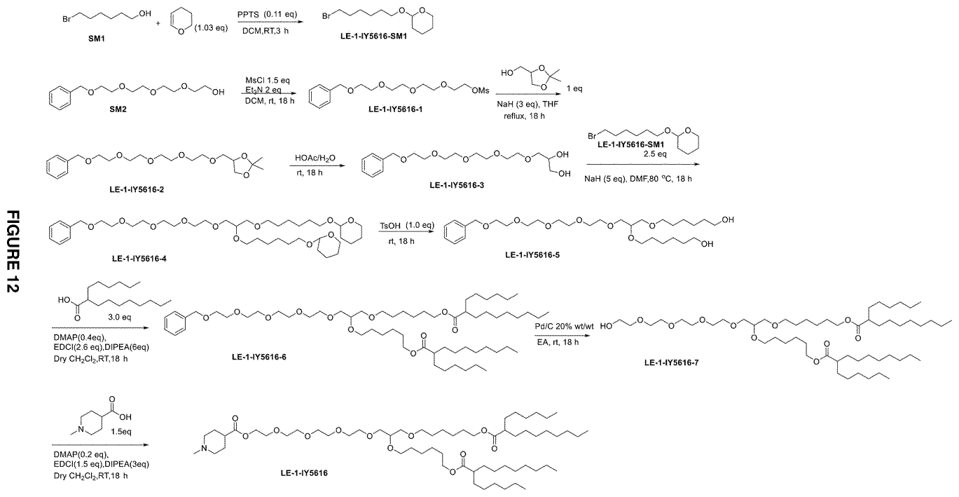

The compound XVII is prepared according to the schema of synthesis of FIG. 11.

### Synthesis of EXP-21-IY5627

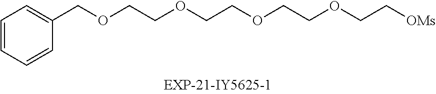 [SMILES: CS(=O)(=O)OCCOCCOCCOCCOCc1ccccc1]

To a mixture of 2-[2-[2-(2-benzyloxyethoxy) ethoxy]ethoxy] ethanol (50 g, 176 mmol) and Et₃N (35.6 g, 352 mmol) in DCM (400 mL) was added methanesulfonyl chloride (30.2 g, 264 mmol) slowly at 0° C. The mixture was stirred overnight at room temperature. CH₂Cl₂ (400 mL) were added to the solution, and the mixture was washed with diluted HCl (1M, 1000 mL). The mixture was shaken, the layers were separated, and the organic layer was collected. The organic layer was further washed with Water (1000 mL) and brine (1000 mL) and dried over Na2SO4. Solvent was then removed to give 2-[2-[2-(2-benzyloxyethoxy) ethoxy]ethoxy]ethyl methanesulfonate (64.1 g, 172 mmol, 97.5% yield) as an orange oil.

1H NMR (400 MHZ, CDCl3) δ 7.37-7.27 (m, 5H), 4.56 (s, 2H), 4.40-4.33 (m, 2H), 3.78-3.72 (m, 2H), 3.69-3.60 (m, 12H), 3.06 (s, 3H).

### Synthesis of EXP-21-IY5625-2

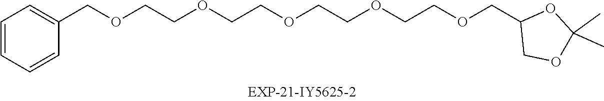 [SMILES: CC1(C)OCC(COCCOCCOCCOCCOCc2ccccc2)O1]

To the solution of (2,2-dimethyl-1,3-dioxolan-4-yl) methanol (24.6 g, 177 mmol) in THF (500 mL) was added NaH (14.1 g, 354 mmol) and the mixture was heated to 80° C. for 30 min. Then the reaction was cooled to room temperature and 2-[2-[2-(2-benzyloxyethoxy) ethoxy]ethoxy]ethyl methanesulfonate (64.1 g, 177 mmol) was added under nitrogen and the reaction was heated at 80° C. for 24 h. TLC indicated that the starting material was consumed. The reaction was quenched with water (300 mL) and extracted with ethyl acetate (600 mL). The aqueous layer was extracted with ethyl acetate (EA) (600 mL) again. The combined organic layers were washed with brine, dried over sodium sulfate, filtered and concentrated. The residue was purified through flash chromatography eluted with 20 to 50% ethyl acetate in petroleum ether to give 24-[2-[2-[2-(2-benzyloxyethoxy) ethoxy]ethoxy] ethoxymethyl]-2,2-dimethyl-1,3-dioxolane (48.635 g, 116 mmol, 65.6% yield) as light yellow oil.

LCMS; Find peak MS (ESI) m/z=421.4 (M±23)⁺ at 2.154 1H NMR (500 MHZ, CDCl3) δ 7.36-7.26 (m, 5H), 4.57 (s, 2H), 4.32-4.22 (m, 1H), 4.07-4.01 (m, 1H), 3.75-3.70 (m, 1H), 3.69-3.60 (m, 16H), 3.59-3.55 (m, 1H), 3.51-3.47 (m, 1H), 1.42 (s, 3H), 1.35 (s, 3H).

### Synthesis of EXP-21-IY5625-3

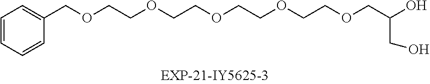 [SMILES: OCC(O)COCCOCCOCCOCCOCc1ccccc1]

The mixture of 4-[2-[2-[2-(2-benzyloxyethoxy) ethoxy]ethoxy] ethoxymethyl]-2,2-dimethyl-1,3-dioxolane (48.635 g, 122 mmol) in AcOH (200 mL) and H₂O (200 mL) was stirred at room temperature for 18 h. TLC (EA/PE 1/1, SM Rf: 0.5; product, Rf: 0.1) indicated that all the starting materials was consumed. The solvent was removed under vacuum and azeotroped with toluene several times. 2-[2-[2-(2-methylsulfonyloxyethoxy) ethoxy]ethoxy]ethyl methanesulfonate (43.7 g, 116 mmol, quant.) as light yellow oil was obtained which was used without further purification. 1H NMR (400 MHZ, CDCl3) δ 7.36-7.23 (m, 5H), 4.56 (s, 2H), 3.89-3.80 (m, 1H), 3.72-3.49 (m, 21H).

### Synthesis of EXP-21-IY5625-4

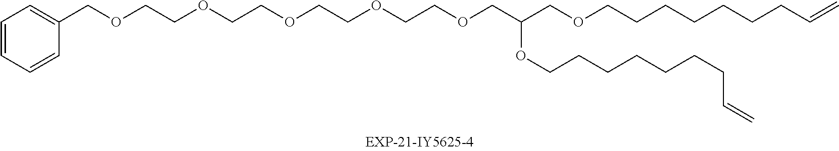 [SMILES: C=CCCCCCCCOCC(COCCOCCOCCOCCOCc1ccccc1)OCCCCCCCC=C]

To a solution of 3-[2-[2-[2-(2-benzyloxyethoxy) ethoxy]ethoxy] ethoxy]propane-1,2-diol (24 g, 60.3 mmol) in dry DMF (200 mL) under nitrogen was added NaH (9.64 g, 241 mmol) and the mixture was heated at 80° C. for 15 min. Then the reaction was cooled to room temperature and 9-bromonon-1-ene (31.9 g, 151 mmol) was added dropwise to this solution. The mixture was stirred at room temperature for 30 min and then at 80° C. for 18 h. TLC (EA/PE=1/1, Rf: 0.5) indicated that a new spot was formed. The reaction was quenched with water (50 mL) and then partitioned between ethyl acetate and water. The aqueous layer was extracted with ethyl acetate again. The combined organic layers were dried over sodium sulfate, filtered and concentrated. The residue was purified by flash chromatography eluted with 20% to 50% ethyl acetate ir petroleum ether to give 2-[2-[2-[2-[2,3-bis (non-8-enoxy) propoxy] ethoxylethoxy]ethoxy] ethoxymethylbenzene (13.24 g, 20.7 mmol, 32.6% yield) as light yellow oil.

1H NMR (500 MHZ, CDCl3) δ 7.36-7.27 (m, 5H), 5.86-5.75 (m, 2H), 5.03-4.89 (m, 4H), 4.57 (s, 2H), 3.69-3.61 (m, 17H), 3.58-3.40 (m, 9H), 2.08-1.99 (m, 4H), 1.61-1.51 (m, 4H), 1.42-1.27 (m, 16H).

### Synthesis of EXP-21-IY5625-5

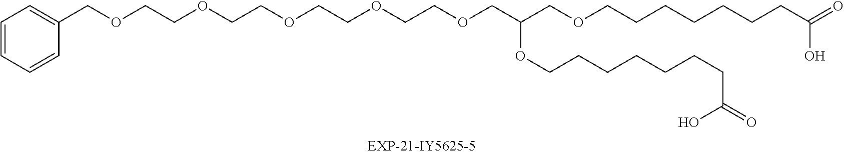 [SMILES: O=C(O)CCCCCCCOCC(COCCOCCOCCOCCOCc1ccccc1)OCCCCCCCC(=O)O]

To a solution of 2-[2-[2-[2-[2,3-bis (non-8-enoxy) propoxy] ethoxy]ethoxy] ethoxy]ethoxymethyl benzene (13.24 g, 20.7 mmol) in MeCN (120 mL), CCl₄ (120 mL) and water (120 mL) was added NalO₄ (35.5 g, 166 mmol) and RuCl₃ (935 mg, 4.15 mmol). The reaction mixture was stirred at room temperature for 18 h. LCMS indicated that the title compound was the major product along with partial mono-aldehyde product. The reaction was filtered and the filtrate was diluted with ethyl acetate (800 mL) and washed with 1N aq. HCl (400 mL). The organic layer was washed with Na2S₂O₃ solution and then dried over sodium sulfate, filtered and concentrated to give 8-[3-[2-[2-[2-(2-benzyloxyethoxy) ethoxy]ethoxy] ethoxy]-2-(7-carboxyheptoxy) propoxy] octanoic acid (13.3 g, 8.28 mmol, 39.9% yield) as pale green oil which was used without further purification.

1H NMR (500 MHZ, CDCl3) δ 9.79-9.72 (m, 1H), 7.36-7.27 (m, 5H), 4.57 (s, 2H), 3.72-3.37 (m, 26H), 2.45-2.39 (m, 1H), 2.32 (t, J=7.3 Hz, 2H), 1.68-1.49 (m, 8H), 1.32 (s, 12H).

8-[3-[2-[2-[2-(2-benzyloxyethoxy) ethoxy]ethoxy] ethoxy]-2-(8-oxooctoxy) propoxy] octanoic acid (13.3 g, 12.7 mmol, 60% purity) was dissolved in t-BuOH: H₂O (3:1, 100 mL), containing NaH₂PO₄.2H₂O (7.64 g, 63.7 mmol), 2-methy-2-butene (60 mL) and sodium chlorite (7.2 g, 63.7 mmol). The reaction was stirred for 2 h at room temperature and LCMS indicated that the starting material was consumed. The reaction mixture was diluted with ethyl acetate. The aqueous layer was extracted with ethyl acetate. The combined extract was dried over sodium sulfate. The residue was purified by flash chromatography eluted with 0% to 5% CH₃OH in DCM to give 8-[3-[2-[2-[2-(2-benzyloxyethoxy) ethoxy]ethoxy] ethoxy]-2-(7-carboxyheptoxy) propoxy] octanoic acid (6.65 g, 9.83 mmol, 77.2% yield) and partial oxidation product (2.78 g) as light yellow oil.

1H NMR (500 MHZ, CDCl3) δ 7.37-7.27 (m, 5H), 4.57 (s, 2H), 3.69-3.61 (m, 16H), 3.58-3.40 (m, 10H), 2.38-2.25 (m, 4H), 1.68-1.48 (m, 8H), 1.34 (d, J=14.9 Hz, 12H).

### Synthesis of EXP-21-IY5625-6

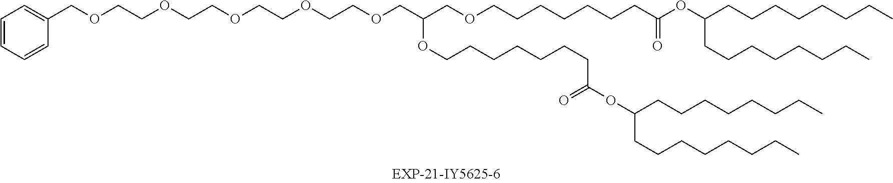 [SMILES: CCCCCCCCC(CCCCCCCC)OC(=O)CCCCCCCOCC(COCCOCCOCCOCCOCc1ccccc1)OCCCCCCCC(=O)OC(CCCCCCCC)CCCCCCCC]

A mixture of 8-[3-[2-[2-[2-(2-benzyloxyethoxy) ethoxy]ethoxy] ethoxy]-2-(7-carboxyheptoxy) propoxy] octanoic acid (6.65 g, 10.3 mmol), heptadecan-9-ol (7.96 g, 31 mmol), DMAP (506 mg, 4.14 mmol), EDCl HCl (5.16 g, 26.9 mmol) and DIEA (8.02 g, 62.1 mmol) in Dry DCM (100 mL). The mixture was stirred for 16 h at room temperature. The mixture was added DCM (500 mL) and washed with 1 N HCl, NaCl and concentrated. The residue was purified by flash column chromatography on silica gel eluting with CH₃OH in DCM (0 to 4%) to give 1-octylnonyl 8-[3-[2-[2-[2-(2-benzyloxyethoxy) ethoxy]ethoxy] ethoxy]-2-[8-(1-octylnonoxy)-8-oxo-octoxy] propoxy] octanoate (3.6 g, 3.05 mmol, 30.5% yield) as pale yellow oil.

1H NMR (400 MHZ, CDCl3) δ 7.36-7.27 (m, 5H), 4.90-4.81 (m, 2H), 4.57 (s, 2H), 3.71-3.61 (m, 16H), 3.59-3.39 (m, 9H), 2.27 (t, J=7.1 Hz, 4H), 1.62-1.46 (m, 16H), 1.28 (d, J=22.7 Hz, 60H), 0.91-0.84 (m, 12H).

### Synthesis of EXP-21-IY5625-7

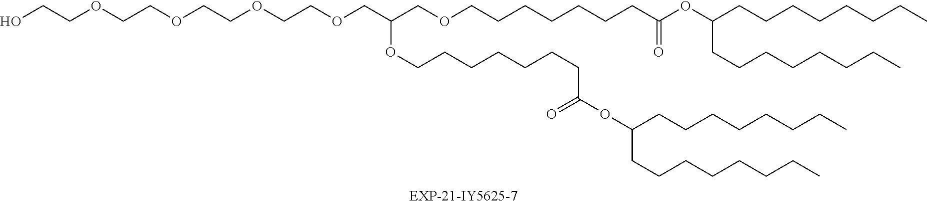 [SMILES: CCCCCCCCC(CCCCCCCC)OC(=O)CCCCCCCOCC(COCCOCCOCCOCCO)OCCCCCCCC(=O)OC(CCCCCCCC)CCCCCCCC]

To the solution of 1-octylnonyl 8-[3-[2-[2-[2-(2-benzyloxyethoxy) ethoxy]ethoxy] ethoxy]-2-[8-(1-octylnonoxy)-8-oxo-octoxy] propoxy] octanoate (3.6 g, 3.22 mmol) in ethyl acetate (50 mL) was added Pd/C (1.1 g, 30% wt/wt). The mixture was stirred at room temperature under hydrogen for 18 h. TLC (CH₃OH/DCM (3%)) indicated that the starting material was consumed. The reaction was filtered through celite and washed with ethyl acetate to give 1-octylnonyl 8-[3-[2-[2-[2-(2-hydroxy ethoxy) ethoxy]ethoxy] ethoxy]-2-[8-(1-octylnonoxy)-8-oxo-octoxy] propoxy] octanoate (3.187 g, 3.1 mmol, 96.3% yield) as colorless oil.

1H NMR (400 MHZ, CDCl3) δ 4.91-4.80 (m, 2H), 3.75-3.71 (m, 2H), 3.70-3.39 (m, 23H), 2.27 (t, J=7.5 Hz, 4H), 1.67-1.45 (m, 16H), 1.35-1.20 (m, 60H), 0.92-0.81 (m, 12H).

### Synthesis of Compound XVI

To a solution of 1-octylnonyl 8-[3-[2-[2-[2-(2-hydroxyethoxy) ethoxy]ethoxy] ethoxy]-2-[8-(1-octylnonoxy)-8-oxo-octoxy] propoxy] octanoate (0.500 g, 0.486 mmol) and 1,3-dimethylpyrrolidine-3-carboxylic acid (0.209 g, 1.46 mmol) in dry dichloromethane (15 mL) was added DIPEA (0.188 g, 1.46 mmol) and DMAP (0.018 g, 0.146 mmol). Then added to EDCl (0.279 g, 1.46 mmol) at 0° C. The reaction was allowed to stir at room temperature for 18 h. The reaction was diluted with dichloromethane and washed with saturated sodium bicarbonate. The organic layer was separated, washed with brine and dried over Na2SO4. The organic layer was filtered and evaporated under vacuum. The residue was purified by silica gel chromatography (0-5% CH₃OH in DCM (4%)) to obtain 2-[2-[2-[2-[2,3-bis [8-(1-octylnonoxy)-8-oxo-octoxy] propoxy] ethoxy]ethoxy] ethoxy]ethyl 1,3-dimethylpyrrolidine-3-carboxylate (0.2761 g, 0.230 mmol, 47.3% yield) as pale yellow oil.

LCMSA; Find peak MS (ESI) m/z=1155.9 (M+H)⁺ at 4.60 min

1H NMR (500 MHZ, CDCl3) δ 4.91-4.82 (m, 2H), 4.31-4.22 (m, 2H), 3.70 (t, J=4.9 Hz, 2H), 3.64 (d, J=6.9 Hz, 12H), 3.60-3.39 (m, 9H), 3.03 (d, J=8.2 Hz, 1H), 2.66 (t, J=25.5 Hz, 2H), 2.55-2.41 (m, 2H), 2.38 (s, 3H), 2.30-2.23 (m, 4H), 1.64-1.47 (m, 17H), 1.38 (s, 3H), 1.34-1.21 (m, 60H), 0.88 (t, J=6.9 Hz, 12H).

### Example 13: Synthesis of 2-[2-[2-[2-[2,3-bis [8-(1-octylnonoxy)-8-oxo-octoxy] propoxy] ethoxy]ethoxy] ethoxy]ethyl 1-methylpiperidine-3-carboxylate (compound XVIII)

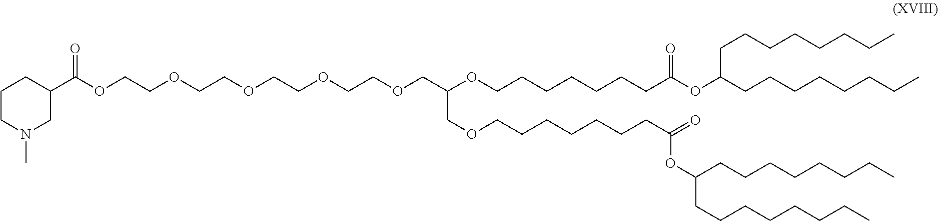 [SMILES: CCCCCCCCC(CCCCCCCC)OC(=O)CCCCCCCOCC(COCCOCCOCCOCCOC(=O)C1CCCN(C)C1)OCCCCCCCC(=O)OC(CCCCCCCC)CCCCCCCC]

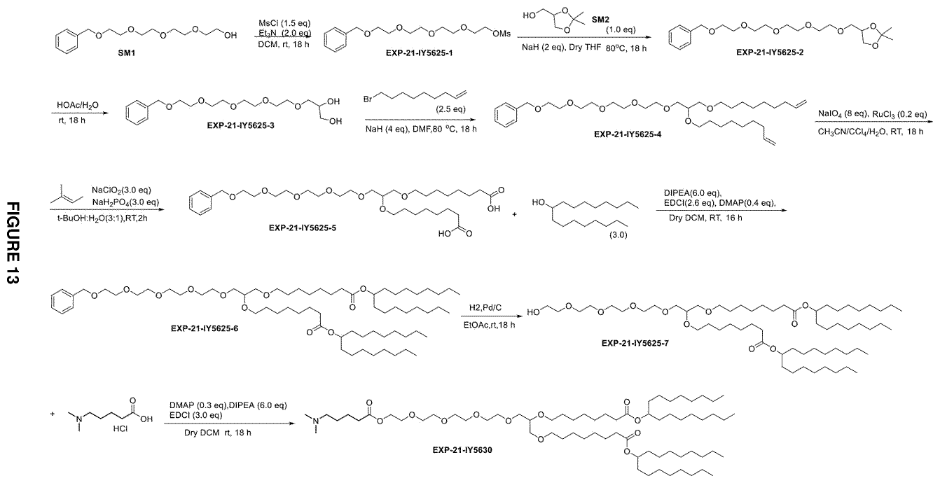

The compound XVIII is prepared according to the schema of synthesis of FIG. 12, from intermediate 1-octylnonyl 8-[3-[2-[2-[2-(2-hydroxyethoxy) ethoxy]ethoxy] ethoxy]-2-[8-(1-octylnonoxy)-8-oxo-octoxy] propoxy] octanoate.

### Synthesis of 1-octylnonyl 8-[3-[2-[2-[2-(2-hydroxyethoxy) ethoxy]ethoxylethoxy]-2-[8-(1-octylnonoxy)-8-oxooctoxy] propoxyloctanoate

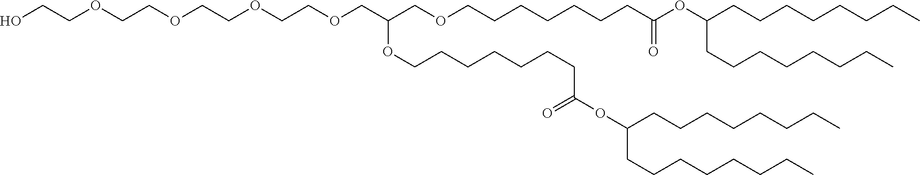 [SMILES: CCCCCCCCC(CCCCCCCC)OC(=O)CCCCCCCOCC(COCCOCCOCCOCCO)OCCCCCCCC(=O)OC(CCCCCCCC)CCCCCCCC]

To the solution of 1-octylnonyl 8-[3-[2-[2-[2-(2-benzyloxyethoxy) ethoxy]ethoxy] ethoxy]-2-[8-(1-octylnonoxy)-8-oxooctoxy] propoxy] octanoate (3.6 g, 3.22 mmol) in ethyl acetate (50 mL) was added Pd/C (1.1 g, 30% wt/wt). The mixture was stirred at room temperature under hydrogen for 18 h. TLC (CH₃OH/DCM (3%)) indicated that the starting material was consumed. The reaction was filtered through celite and washed with ethyl acetate to give 1-octylnonyl 8-[3-[2-[2-[2-(2-hydroxyethoxy) ethoxy]ethoxy] ethoxy]-2-[8-(1-octylnonoxy)-8-oxooctoxy] propoxy] octanoate (3.187 g, 3.1 mmol, 96.3% yield) as colorless oil.

1H NMR (400 MHZ, CDCl3) δ 4.91-4.80 (m, 2H), 3.75-3.71 (m, 2H), 3.70-3.39 (m, 23H), 2.27 (t, J=7.5 Hz, 4H), 1.67-1.45 (m, 16H), 1.35-1.20 (m, 60H), 0.92-0.81 (m, 12H).

### Synthesis of the Compound XVIII

To a solution of 1-octylnonyl 8-[3-[2-[2-[2-(2-hydroxyethoxy) ethoxy]ethoxy] ethoxy]-2-[8-(1-octylnonoxy)-8-oxooctoxy] propoxy] octanoate (0.500 g, 0.486 mmol) and 1-methylpiperidine-3-carboxylic acid (0.209 g, 1.46 mmol) in dry dichloromethane (15 mL) was added DIPEA (0.188 g, 1.46 mmol) and DMAP (0.018 g, 0.146 mmol). Then added to EDCl (0.279 g, 1.46 mmol) at 0° C. The reaction was allowed to stir at room temperature for 18 h. The reaction was diluted with dichloromethane and washed with saturated sodium bicarbonate. The organic layer was separated, washed with brine and dried over Na2SO4. The organic layer was filtered and evaporated under vacuum. The residue was purified by silica gel chromatography (0-5% CH₃OH in DCM (4%)) to obtain2-[2-[2-[2-[2,3-bis [8-(1-octylnonoxy)-8-oxo-octoxy] propoxy] ethoxy]ethoxy] ethoxy]ethyl 1-methylpiperidine-3-carboxylate (0.3595 g, 0.299 mmol, 61.5% yield) as colorless oil.

Find peak MS (ESI) m/z=1155.0 (M+H)⁺ at 3.88 min

1H NMR (500 MHZ, CDCl3) δ 4.91-4.81 (m, 2H), 4.29-4.19 (m, 2H), 3.74-3.60 (m, 14H), 3.59-3.36 (m, 9H), 2.96 (d, J=9.7 Hz, 1H), 2.68 (d, J=36.1 Hz, 2H), 2.34-2.22 (m, 7H), 2.17 (d, J=10.7 Hz, 1H), 2.03-1.92 (m, 2H), 1.77-1.71 (m, 1H), 1.65-1.45 (m, 17H), 1.42 (d, J=6.7 Hz, 1H), 1.34-1.22 (m, 60H), 0.88 (t, J=6.9 Hz, 12H).

### Example 14: Synthesis of 2-[2-[2-[2-[2,3-bis [6-(2-hexyldecanoyloxy) hexoxy] propoxy] ethoxy]ethoxy] ethoxy]ethyl 1-methylpiperidine-4-carboxylate (compound XIX)

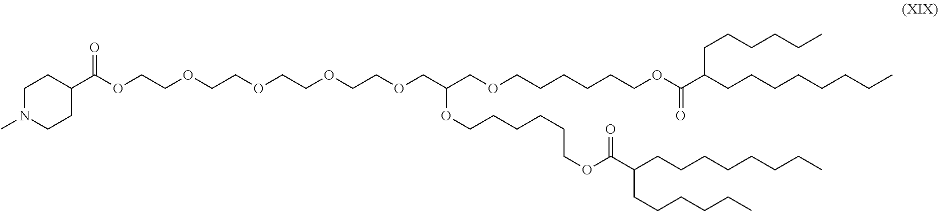 [SMILES: CCCCCCCCC(CCCCCC)C(=O)OCCCCCCOCC(COCCOCCOCCOCCOC(=O)C1CCN(C)CC1)OCCCCCCOC(=O)C(CCCCCC)CCCCCCCC]

The compound XIX is prepared according to the schema of synthesis of FIG. 12.

### Synthesis of IY5616-SM1

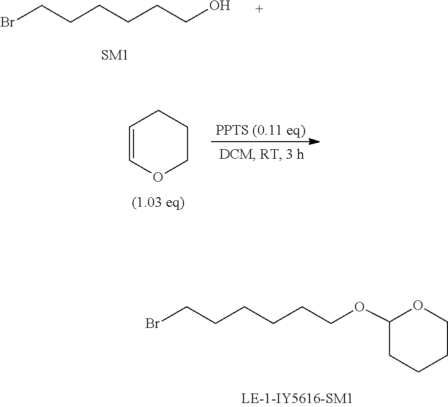 [SMILES: OCCCCCCBr ; C1=COCCC1 ; "PPTS (0.11 eq)" ; BrCCCCCCOC1CCCCO1]

To a solution of 6-bromohexan-1-ol (18 g, 0.10 mol) and 3,4-dihydro-2H-pyran (8.61 g, 0.1 mol) in DCM (500 mL) was added pyridinium p-toluenesulfonate (PPTS) (2.75 g, 0.01 mol) then stirred at room temperature for 3 h. TLC (EA/PE 1/9, SM Rf: 0.2; product, Rf: 0.7) indicated that all the starting materials was consumed. The solvent was concentrated and purified by flash chromatography column (0-10% EA in PE (5%) to give 2-(6-bromohexoxy) tetrahydropyran (20.3 g, 77.0% yield) as colorless oil.

1H NMR (400 MHZ, CDCl3) δ 4.57 (t, J=3.6 Hz, 1H), 3.92-3.82 (m, 1H), 3.79-3.69 (m, 1H), 3.56-3.46 (m, 1H), 3.46-3.34 (m, 3H), 1.94-1.77 (m, 3H), 1.75-1.69 (m, 1H), 1.66-1.35 (m, 10H).

### Synthesis of IY5616-1

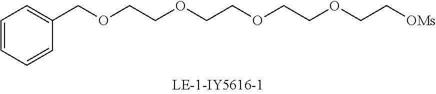 [SMILES: CS(=O)(=O)OCCOCCOCCOCCOCc1ccccc1]

2-[2-[2-(2-benzyloxyethoxy) ethoxy]ethoxy] ethanol (10 g, 35.2 mmol) and triethylamine (7.12 g, 70.3 mmol) in dry dichloromethane (100 mL) at 0° C. Methanesulfonyl chloride (6.04 g, 52.8 mmol) in dry DCM (10 mL) was added dropwise to this solution at 0° C. The mixture was allowed to warm to room temperature and stirred at room temperature for 18 h. Triethylamine hydrochloride was filtered off, and the DCM solution was washed with 0.1 N HCl and dried over sodium sulfate. Removing the solvent afforded 2-[2-[2-(2-benzyloxyethoxy) ethoxy]ethoxy]ethyl methanesulfonate (12.8 g, quant.) as light yellow oil which was used without further purification.

1H NMR (400 MHZ, CDCl3) δ 7.42-7.27 (m, 5H), 4.57 (s, 2H), 4.41-4.31 (m, 2H), 3.79-3.72 (m, 2H), 3.70-3.60 (m, 12H), 3.05 (d, J=9.8 Hz, 3H).

### Synthesis of IY5616-2

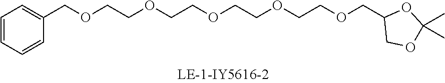 [SMILES: CC1(C)OCC(COCCOCCOCCOCCOCc2ccccc2)O1]

Bromomethylbenzene (12.8 g, 35.5 mmol) in THF (100 ml) was added to NaH (4.24 g, 0.106 mol) in THF (50 ml) at 0° C. The mixture was stirred at 0° C. for 30 min. (2,2-dimethyl-1,3-dioxolan-4-yl) methanol (4.67 g, 35.3 mmol) was added to this mixture. The mixture was stirred at 0° C. for 1 hr and then rise to 75° C. for 18 hr. The mixture was quenched with water and extracted with ethyl acetate (EA) (500 ml). The organic was washed with brine, dried over sodium sulfate, filtered and concentrated. The residue was purified by flash chromatography column (5% to 10% (8%) MeOH in DCM) to give 4-(benzyloxymethyl)-2,2-dimethyl-1,3-dioxolane (10.64 g, yield 75.6%) as a light-yellow oil.

1H NMR (400 MHZ, CDCl3) δ 7.37-7.23 (m, 5H), 4.56 (s, 2H), 4.31-4.21 (m, 1H), 4.08-4.00 (m, 1H), 3.75-3.70 (m, 1H), 3.69-3.61 (m, 16H), 3.60-3.54 (m, 1H), 3.53-3.46 (m, 1H), 1.45-1.40 (m, 3H), 1.36 (d, J=7.7 Hz, 3H).

### Synthesis of IY5616-3

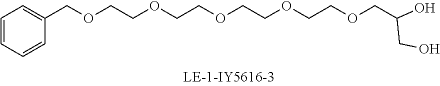 [SMILES: OCC(O)COCCOCCOCCOCCOCc1ccccc1]

The mixture of 4-[2-[2-[2-(2-benzyloxyethoxy) ethoxy]ethoxy] ethoxymethyl]-2,2-dimethyl-1,3-dioxolane (10.6 g, 26.7 mmol) in AcOH (50 mL) and water (50 mL). The mixture was stirred for 16 h at ambient temperature. TLC (EA/PE 1/1, SM Rf: 0.5; product, Rf: 0.1) indicated that all the starting materials was consumed. The solvent was removed under vacuum and azeotroped with toluene several times. 2-[2-[2-(2-methylsulfonyloxyethoxy) ethoxy]ethoxy]ethyl methanesulfonate (10.0 g, quant.) as light yellow oil was obtained which was used without further purification.

1H NMR (400 MHZ, CDCl3) δ 7.34-7.13 (m, 5H), 4.56 (s, 2H), 3.89-3.81 (m, 1H), 3.68-3.52 (m, 20H).

### Synthesis of IY5616-4

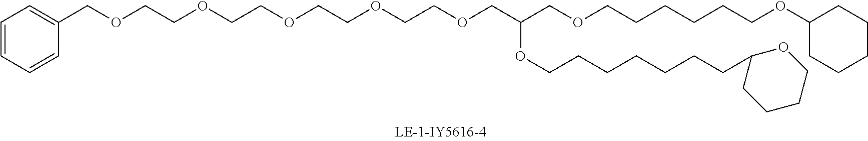 [SMILES: c1ccc(COCCOCCOCCOCCOCC(COCCCCCCOC2CCCCC2)OCCCCCCCC2CCCCO2)cc1]

To the solution of 3-[2-[2-[2-(2-benzyloxyethoxy) ethoxy]ethoxy] ethoxy]propane-1,2-diol (10.0 g, 27.9 mmol) in dry DMF (120 mL) was added NaH (5.58 g, 139 mmol) several times at 0° C. then the mixture was heated to 80° C. for 30 min. Then the reaction was cooled to room temperature and 2-(6-bromohexoxy) tetrahydropyran (18.5 g, 69.7 mmol) in dry DMF (30 mL) was added under nitrogen and the reaction was heated at 80° C. for 18 h. TLC indicated that the starting material was consumed. The reaction was quenched with water and extracted with ethyl acetate. The aqueous layer was extracted with ethyl acetate again. The combined organic layers were washed with brine, dried over sodium sulfate, filtered and concentrated. The residue was purified through flash chromatography eluted with 0 to 5% (1%) methanol in DCM to give 2-[6-[1-[2-[2-[2-(2-benzyloxyethoxy) ethoxy]ethoxy] ethoxymethyl]-2-(6-tetrahydropyran-2-yloxyhexoxy) ethoxy] hexoxy] tetrahydropyran (8.0 g, 39.4% yield) as light yellow oil.

1H NMR (400 MHZ, CDCl3) δ 7.37-7.24 (m, 5H), 4.57 (s, 4H), 3.90-3.83 (m, 2H), 3.78-3.33 (m, 31H), 1.79-1.67 (m, 3H), 1.62-1.50 (m, 17H), 1.41-1.34 (m, 8H).

### Synthesis of IY5616-5

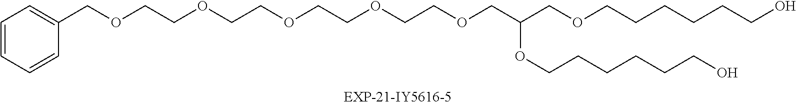 [SMILES: OCCCCCCOCC(COCCOCCOCCOCCOCc1ccccc1)OCCCCCCO]

To a solution of 2-[6-[1-[2-[2-[2-(2-benzyloxyethoxy) ethoxy]ethoxy] ethoxymethyl]-2-(6-tetrahydropyran-2-yloxyhexoxy) ethoxy] hexoxy] tetrahydropyran (8.0 g, 11.0 mmol) in EtOH (120 mL) was added p-Toluenesulfonic acid (2.1 g, 12.1 mmol) in one portion at room temperature and the mixture was stirred at room temperature for 24 h. TLC (4% CH₃OH in DCM) indicated that the starting material was disappeared completely. After the reaction was quenched with dilute sodium bicarbonate solution (150 mL), the solvent was extracted with EA (2*100 mL). The organic layers were washed with brine, dried over sodium sulfate, filtered and concentrated. The residue was purified by flash chromatography eluted with 0% to 10% CH₃OH in DCM (8%) to give 6-[3-[2-[2-[2-(2-benzyloxyethoxy) ethoxy]ethoxy] ethoxy]-2-(6-hydroxyhexoxy) propoxy] hexan-1-ol (3.23 g, 52.5% yield) as pale yellow oil. 1H NMR (500 MHZ, CDCl3) δ 7.40-7.20 (m, 5H), 4.57 (s, 2H), 3.72-3.38 (m, 29H), 1.87 (s, 2H), 1.63-1.47 (m, 8H), 1.42-1.28 (m, 8H).

### Synthesis of IY5616-6

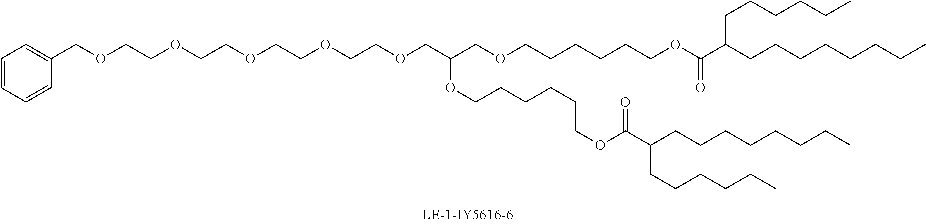 [SMILES: CCCCCCCCC(CCCCCC)C(=O)OCCCCCCOCC(COCCOCCOCCOCCOCc1ccccc1)OCCCCCCOC(=O)C(CCCCCC)CCCCCCCC]

To a solution of 6-[3-[2-[2-[2-(2-benzyloxyethoxy) ethoxy]ethoxy] ethoxy]-2-(6-hydroxyhexoxy) propoxy] hexan-1-ol (1.6 g, 2.86 mmol) in dry DCM (20 mL) was added 2-hexyldecanoic acid (2.2 g, 8.59 mmol), DIPEA (2.22 g, 17.2 mmol) and DMAP (0.14 g, 1.15 mmol) at room temperature. Then the mixture was cooled to 0° C. and portion wise over 15 min added EDCl (1.43 g, 7.45 mmol). The reaction was stirred for 17 hours at room temperature. TLC (5% CH₃OH in DCM) shows that the reaction was finished. The reaction was poured into water and extracted with DCM. The water was extracted one more time with DCM. The combined organics were washed with brine, dried over Na2SO4 and concentrated under vacuum. The residue was purified by column chromatography with CH3OH in DCM (0-4%) (3%) to give 6-[3-[2-[2-[2-(2-benzyloxyethoxy) ethoxy]ethoxy] ethoxy]-2-[6-(2-hexyldecanoyloxy) hexoxy] propoxy] hexyl 2-hexyldecanoate (2.9 g, 97.8% yield) as light yellow oil.

1H NMR (400 MHZ, CDCl3) δ 7.38-7.27 (m, 5H), 4.57 (s, 2H), 4.09-4.03 (m, 4H), 3.69-3.40 (m, 25H), 2.34-2.27 (m, 2H), 1.63-1.34 (m, 24H), 1.25 (s, 40H), 0.87 (t, J=6.5 Hz, 12H).

### Synthesis of IY5616-7

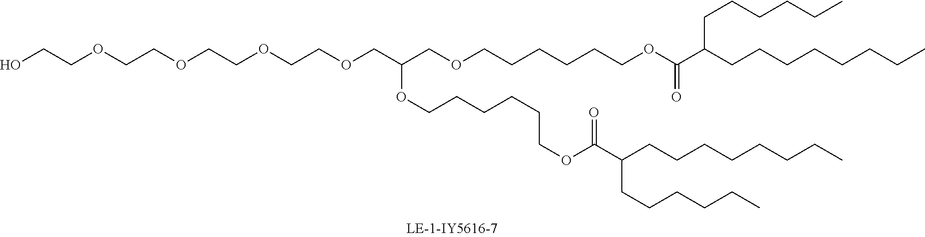 [SMILES: CCCCCCCCC(CCCCCC)C(=O)OCCCCCCOCC(COCCOCCOCCOCCO)OCCCCCCOC(=O)C(CCCCCC)CCCCCCCC]

A solution of 6-[3-[2-[2-[2-(2-benzyloxyethoxy) ethoxy]ethoxy] ethoxy]-2-[6-(2-hexyldecanoyloxy) hexoxy] propoxy] hexyl 2-hexyldecanoate (2.90 g, 2.80 mmol) in EtOAc (30 mL) was purged for 10 minutes with N2 followed by addition of Pd/C (20% wt/wt, 0.6 g) and the reaction continued purging with N2. The reaction was next evacuated under vacuum and backfilled with H_{2 3} times. The reaction was next stirred overnight at room temperature under an atmosphere of H₂. TLC shows that the reaction was finished. The slurry filtered through celite and the celite was rinsed with EtOAc several times. The combined organics were next concentrated under vacuum to give 6-[2-[6-(2-hexyldecanoyloxy) hexoxy]-3-[2-[2-[2-(2-hydroxyethoxy) ethoxy]ethoxy] ethoxy] propoxy] hexyl 2-hexyldecanoate (1.7 g, 64.2% yield) as a light yellow oil.

1H NMR (400 MHZ, CDCl3) δ 7.38-7.27 (m, 5H), 4.57 (s, 2H), 4.09-4.03 (m, 4H), 3.69-3.40 (m, 25H), 2.34-2.27 (m, 2H), 1.63-1.34 (m, 24H), 1.25 (s, 40H), 0.87 (t, J=6.5 Hz, 12H).

### Synthesis of Compound XIX

To a solution of 6-[2-[6-(2-hexyldecanoyloxy) hexoxy]-3-[2-[2-[2-(2-hydroxyethoxy) ethoxy]ethoxy] ethoxy] propoxy] hexyl 2-hexyldecanoate (1.0 g, 1.06 mmol) in dry DCM (20 mL) was added 1-methylpiperidine-4-carboxylic acid (0.23 g, 1.59 mmol), DIPEA (0.41 g, 3.17 mmol) and DMAP (0.03 g, 0.21 mmol) at room temperature. Then the mixture was cooled to 0° C. and portion wise over 15 min added EDCl (0.30 g, 1.59 mmol). The reaction was stirred for 17 hours at room temperature. TLC shows that the reaction was finished. The reaction was poured into water and extracted with DCM. The water was extracted one more time with DCM. The combined organics were washed with brine, dried over Na₂SO₄ and concentrated under vacuum. Then purified by column chromatography with CH3OH in DCM (0-10%) (8%) to give 2-[2-[2-[2-[2,3-bis [6-(2-hexyldecanoyloxy) hexoxy] propoxy] ethoxy]ethoxy] ethoxy]ethyl 1-methylpiperidine-4-carboxylate (614 mg, 54.2% yield) as light yellow oil.

1H NMR (400 MHZ, CDCl3) δ 4.28-4.21 (m, 2H), 4.10-4.01 (m, 4H), 3.72-3.61 (m, 14H), 3.60-3.40 (m, 9H), 2.83 (d, J=11.5 Hz, 2H), 2.33-2.28 (m, 5H), 2.05 (s, 3H), 1.96-1.90 (m, 2H), 1.84-1.76 (m, 2H), 1.65-1.54 (m, 12H), 1.47-1.35 (m, 12H), 1.25 (s, 40H), 0.90-0.85 (m, 12H).

### Example 15: Synthesis of 1-octylnonyl 8-[3-[2-[2-[2-[2-[5-(dimethylamino) pentanoyloxy] ethoxy]ethoxy] ethoxy]ethoxy]-2-[8-(1-octylnonoxy)-8-oxo-octoxy] propoxyloctanoate (compound XX)

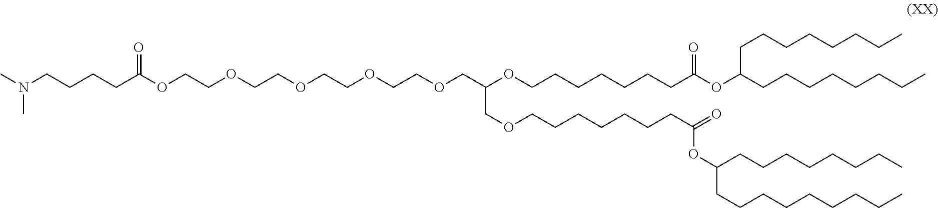 [SMILES: CCCCCCCCCC(CCCCCCCC)OC(=O)CCCCCCCOCC(COCCOCCOCCOCCOC(=O)CCCCN(C)C)OCCCCCCCC(=O)OC(CCCCCCCC)CCCCCCCC]

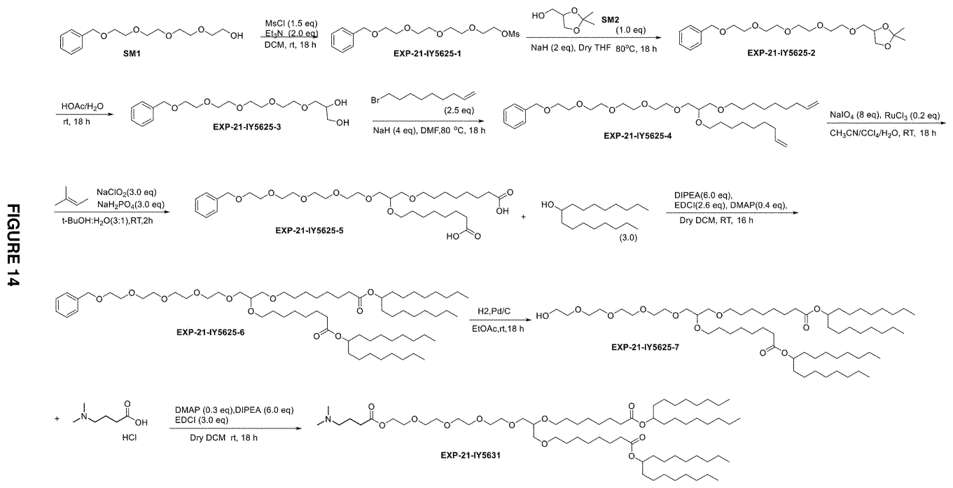

The compound XX is prepared according to the schema of synthesis of FIG. 13.

### Synthesis of EXP-21-IY5625-1

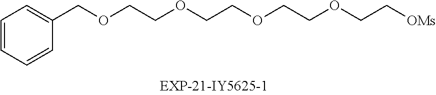 [SMILES: CS(=O)(=O)OCCOCCOCCOCCOCc1ccccc1]

To a mixture of 2-[2-[2-(2-benzyloxyethoxy) ethoxy]ethoxy] ethanol (50 g, 176 mmol) and Et₃N (35.6 g, 352 mmol) in DCM (400 mL) was added methanesulfonyl chloride (30.2 g, 264 mmol) slowly at 0° C. The mixture was stirred overnight at room temperature. CH₂Cl₂ (400 mL) were added to the solution, and the mixture was washed with diluted HCl (1M, 1000 mL). The mixture was shaken, the layers were separated, and the organic layer was collected. The organic layer was further washed with Water (1000 mL) and brine (1000 mL) and dried over Na₂SO₄. Solvent was then removed to give 2-[2-[2-(2-benzyloxyethoxy) ethoxy]ethoxy]ethyl methanesulfonate (64.1 g, 172 mmol, 97.5% yield) as an orange oil.

¹H NMR (400 MHZ, CDCl3) δ 7.37-7.27 (m, 5H), 4.56 (s, 2H), 4.40-4.33 (m, 2H), 3.78-3.72 (m, 2H), 3.69-3.60 (m, 12H), 3.06 (s, 3H).

### Synthesis of EXP-21-IY5625-2

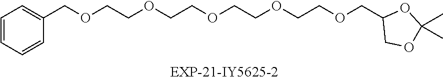 [SMILES: CC1(C)OCC(COCCOCCOCCOCCOCc2ccccc2)O1]

To the solution of (2,2-dimethyl-1,3-dioxolan-4-yl) methanol (24.6 g, 177 mmol) in THF (500 mL) was added NaH (14.1 g, 354 mmol) and the mixture was heated to 80° C. for 30 min. Then the reaction was cooled to room temperature and 2-[2-[2-(2-benzyloxyethoxy) ethoxy]ethoxy]ethyl methanesulfonate (64.1 g, 177 mmol) was added under nitrogen and the reaction was heated at 80° C. for 24 h. TLC indicated that the starting material was consumed. The reaction was quenched with water (300 mL) and extracted with ethyl acetate (600 mL). The aqueous layer was extracted with ethyl acetate (EA) (600 mL) again. The combined organic layers were washed with brine, dried over sodium sulfate, filtered and concentrated. The residue was purified through flash chromatography eluted with 20 to 50% ethyl acetate in petroleum ether to give 24-[2-[2-[2-(2-benzyloxyethoxy) ethoxy]ethoxy] ethoxymethyl]-2,2-dimethyl-1,3-dioxolane (48.635 g, 116 mmol, 65.6% yield) as light yellow oil.

LCMS; Find peak MS (ESI) m/z=421.4 (M±23)⁺ at 2.154

¹H NMR (500 MHZ, CDCl3) δ 7.36-7.26 (m, 5H), 4.57 (s, 2H), 4.32-4.22 (m, 1H), 4.07-4.01 (m, 1H), 3.75-3.70 (m, 1H), 3.69-3.60 (m, 16H), 3.59-3.55 (m, 1H), 3.51-3.47 (m, 1H), 1.42 (s, 3H), 1.35 (s, 3H).

### Synthesis of EXP-21-IY5625-3

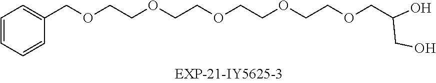 [SMILES: OCC(O)COCCOCCOCCOCCOCc1ccccc1]

The mixture of 4-[2-[2-[2-(2-benzyloxyethoxy) ethoxy]ethoxy] ethoxymethyl]-2,2-dimethyl-1,3-dioxolane (48.635 g, 122 mmol) in AcOH (200 mL) and H₂O (200 mL) was stirred at room temperature for 18 h. TLC (EA/PE 1/1, SM Rf: 0.5; product, Rf: 0.1) indicated that all the starting materials was consumed. The solvent was removed under vacuum and azeotroped with toluene several times. 2-[2-[2-(2-methylsulfonyloxyethoxy) ethoxy]ethoxy]ethyl methanesulfonate (43.7 g, 116 mmol, quant.) as light yellow oil was obtained which was used without further purification.

1H NMR (400 MHZ, CDCl3) δ 7.36-7.23 (m, 5H), 4.56 (s, 2H), 3.89-3.80 (m, 1H), 3.72-3.49 (m, 21H).

### Synthesis of EXP-21-IY5625-4

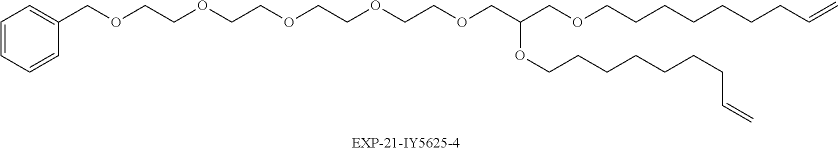 [SMILES: C=CCCCCCCCOCC(COCCOCCOCCOCCOCc1ccccc1)OCCCCCCCC=C]

To a solution of 3-[2-[2-[2-(2-benzyloxyethoxy) ethoxy]ethoxy] ethoxy]propane-1,2-diol (24 g, 60.3 mmol) in dry DMF (200 mL) under nitrogen was added NaH (9.64 g, 241 mmol) and the mixture was heated at 80° C. for 15 min. Then the reaction was cooled to room temperature and 9-bromonon-1-ene (31.9 g, 151 mmol) was added dropwise to this solution. The mixture was stirred at room temperature for 30 min and then at 80° C. for 18 h. TLC (EA/PE=1/1, Rf: 0.5) indicated that a new spot was formed. The reaction was quenched with water (50 mL) and then partitioned between ethyl acetate and water. The aqueous layer was extracted with ethyl acetate again. The combined organic layers were dried over sodium sulfate, filtered and concentrated. The residue was purified by flash chromatography eluted with 20% to 50% ethyl acetate in petroleum ether to give 2-[2-[2-[2-[2,3-bis (non-8-enoxy) propoxy] ethoxy]ethoxy] ethoxy]ethoxymethylbenzene (13.24 g, 20.7 mmol, 32.6% yield) as light yellow oil.

1H NMR (500 MHZ, CDCl3) δ 7.36-7.27 (m, 5H), 5.86-5.75 (m, 2H), 5.03-4.89 (m, 4H), 4.57 (s, 2H), 3.69-3.61 (m, 17H), 3.58-3.40 (m, 9H), 2.08-1.99 (m, 4H), 1.61-1.51 (m, 4H), 1.42-1.27 (m, 16H).

### Synthesis of EXP-21-IY5625-5

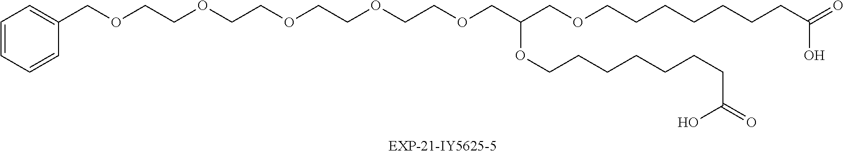 [SMILES: O=C(O)CCCCCCCOCC(COCCOCCOCCOCCOCc1ccccc1)OCCCCCCCC(=O)O]

To a solution of 2-[2-[2-[2-[2,3-bis (non-8-enoxy) propoxy] ethoxy]ethoxy] ethoxy]ethoxymethyl benzene (13.24 g, 20.7 mmol) in MeCN (120 mL), CCl₄ (120 mL) and water (120 mL) was added NalO₄ (35.5 g, 166 mmol) and RuCl₃ (935 mg, 4.15 mmol). The reaction mixture was stirred at room temperature for 18 h. LCMS indicated that the title compound was the major product along with partial mono-aldehyde product. The reaction was filtered, and the filtrate was diluted with ethyl acetate (800 mL) and washed with 1N aq. HCl (400 mL). The organic layer was washed with Na2S₂O₃ solution and then dried over sodium sulfate, filtered and concentrated to give 8-[3-[2-[2-[2-(2-benzyloxyethoxy) ethoxy]ethoxy] ethoxy]-2-(7-carboxyheptoxy) propoxy] octanoic acid (13.3 g, 8.28 mmol, 39.9% yield) as pale green oil which was used without further purification.

1H NMR (500 MHZ, CDCl3) δ 9.79-9.72 (m, 1H), 7.36-7.27 (m, 5H), 4.57 (s, 2H), 3.72-3.37 (m, 26H), 2.45-2.39 (m, 1H), 2.32 (t, J=7.3 Hz, 2H), 1.68-1.49 (m, 8H), 1.32 (s, 12H).

8-[3-[2-[2-[2-(2-benzyloxyethoxy) ethoxy]ethoxy] ethoxy]-2-(8-oxooctoxy) propoxy] octanoic acid (13.3 g, 12.7 mmol, 60% purity) was dissolved in t-BuOH: H₂O (3:1, 100 mL), containing NaH₂PO₄.2H₂O (7.64 g, 63.7 mmol), 2-methy-2-butene (60 mL) and sodium chlorite (7.2 g, 63.7 mmol). The reaction was stirred for 2 h at room temperature and LCMS indicated that the starting material was consumed. The reaction mixture was diluted with ethyl acetate. The aqueous layer was extracted with ethyl acetate. The combined extract was dried over sodium sulfate. The residue was purified by flash chromatography eluted with 0% to 5% CH₃OH in DCM to give 8-[3-[2-[2-[2-(2-benzyloxyethoxy) ethoxy]ethoxy] ethoxy]-2-(7-carboxyheptoxy) propoxy] octanoic acid (6.65 g, 9.83 mmol, 77.2% yield) and partial oxidation product (2.78 g) as light yellow oil. 1H NMR (500 MHZ, CDCl3) δ 7.37-7.27 (m, 5H), 4.57 (s, 2H), 3.69-3.61 (m, 16H), 3.58-3.40 (m, 10H), 2.38-2.25 (m, 4H), 1.68-1.48 (m, 8H), 1.34 (d, J=14.9 Hz, 12H).

### Synthesis of EXP-21-IY5625-6

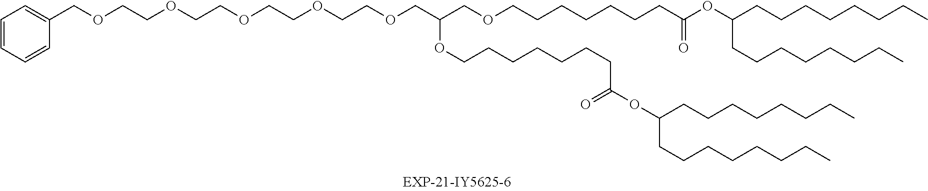 [SMILES: CCCCCCCCC(CCCCCCCC)OC(=O)CCCCCCCOCC(COCCOCCOCCOCCOCc1ccccc1)OCCCCCCCC(=O)OC(CCCCCCCC)CCCCCCCC]

A mixture of 8-[3-[2-[2-[2-(2-benzyloxyethoxy) ethoxy]ethoxy] ethoxy]-2-(7-carboxyheptoxy) propoxy] octanoic acid (6.65 g, 10.3 mmol), heptadecan-9-ol (7.96 g, 31 mmol), DMAP (506 mg, 4.14 mmol), EDCl HCl (5.16 g, 26.9 mmol) and DIEA (8.02 g, 62.1 mmol) in Dry DCM (100 mL). The mixture was stirred for 16 h at room temperature. The mixture was added DCM (500 mL) and washed with 1 N HCl, NaCl and concentrated. The residue was purified by flash column chromatography on silica gel eluting with CH₃OH in DCM (0 to 4%) to give 1-octylnonyl 8-[3-[2-[2-[2-(2-benzyloxyethoxy) ethoxy]ethoxy] ethoxy]-2-[8-(1-octylnonoxy)-8-oxo-octoxy] propoxyloctanoate (3.6 g, 3.05 mmol, 30.5% yield) as pale yellow oil.

¹H NMR (400 MHZ, CDCl3) δ 7.36-7.27 (m, 5H), 4.90-4.81 (m, 2H), 4.57 (s, 2H), 3.71-3.61 (m, 16H), 3.59-3.39 (m, 9H), 2.27 (t, J=7.1 Hz, 4H), 1.62-1.46 (m, 16H), 1.28 (d, J=22.7 Hz, 60H), 0.91-0.84 (m, 12H).

### Synthesis of EXP-21-IY5625-7

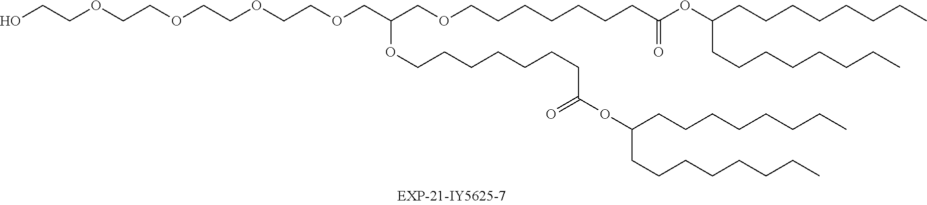 [SMILES: CCCCCCCCC(CCCCCCCC)OC(=O)CCCCCCCOCC(COCCOCCOCCOCCO)OCCCCCCCC(=O)OC(CCCCCCCC)CCCCCCCC]

To the solution of 1-octylnonyl 8-[3-[2-[2-[2-(2-benzyloxyethoxy) ethoxy]ethoxy] ethoxy]-2-[8-(1-octylnonoxy)-8-oxo-octoxy] propoxyloctanoate (3.6 g, 3.22 mmol) in ethyl acetate (50 mL) was added Pd/C (1.1 g, 30% wt/wt). The mixture was stirred at room temperature under hydrogen for 18 h. TLC (CH₃OH/DCM (3%)) indicated that the starting material was consumed. The reaction was filtered through celite and washed with ethyl acetate to give1-octylnonyl 8-[3-[2-[2-[2-(2-hydroxyethoxy) ethoxy]ethoxy] ethoxy]-2-[8-(1-octylnonoxy)-8-oxo-octoxy] propoxyloctanoate (3.187 g, 3.1 mmol, 96.3% yield) as colorless oil.

¹H NMR (400 MHZ, CDCl3) δ 4.91-4.80 (m, 2H), 3.75-3.71 (m, 2H), 3.70-3.39 (m, 23H), 2.27 (t, J=7.5 Hz, 4H), 1.67-1.45 (m, 16H), 1.35-1.20 (m, 60H), 0.92-0.81 (m, 12H).

### Synthesis of Compound XX

To a solution of 1-octylnonyl 8-[3-[2-[2-[2-(2-hydroxyethoxy) ethoxy]ethoxy] ethoxy]-2-[8-(1-octylnonoxy)-8-oxo-octoxy] propoxy] octanoate (0.500 g, 0.486 mmol) and 5-(dimethylamino) pentanoic acid;hydrochloride (0.265 g, 1.46 mmol) in dry dichloromethane (15 mL) was added DIPEA (0.377 g, 2.91 mmol) and DMAP (0.018 g, 0.146 mmol). Then added to EDCl (0.279 g, 1.46 mmol) at 0° C. The reaction was allowed to stir at room temperature for 18 h. The reaction was diluted with dichloromethane and washed with saturated sodium bicarbonate. The organic layer was separated and washed with brine and dried over Na2SO4. The organic layer was filtered and evaporated under vacuum. The residue was purified by silica gel chromatography (0-5% CH₃OH in DCM (4%)) to obtain 1-octylnonyl 8-[3-[2-[2-[2-[2-[5-(dimethylamino) pentanoyloxy] ethoxy]ethoxy] ethoxy]ethoxy]-2-[8-(1-octylnonoxy)-8-oxo-octoxy] propoxyloctanoate (0.2896 g, 0.243 mmol, 50.0% yield) as yellow oil.

LCMS; Find peak MS (ESI) m/z=1157.9 (M+H)⁺ at 4.553 min.

1H NMR (500 MHZ, CDCl3) δ 4.91-4.82 (m, 2H), 4.26-4.19 (m, 2H), 3.75-3.38 (m, 23H), 2.51 (s, 2H), 2.45-2.34 (m, 8H), 2.27 (t, J=7.5 Hz, 4H), 1.69-1.59 (m, 7H), 1.57-1.48 (m, 11H), 1.34-1.21 (m, 62H), 0.88 (t, J=6.9 Hz, 12H).

### Example 16: Synthesis of 1-octylnonyl 8-[3-[2-[2-[2-[2-[4-(dimethylamino) butanoyloxy] ethoxy]ethoxy] ethoxy]ethoxy]-2-[8-(1-octylnonoxy)-8-oxo-octoxy] propoxyloctanoate (compound XXI)

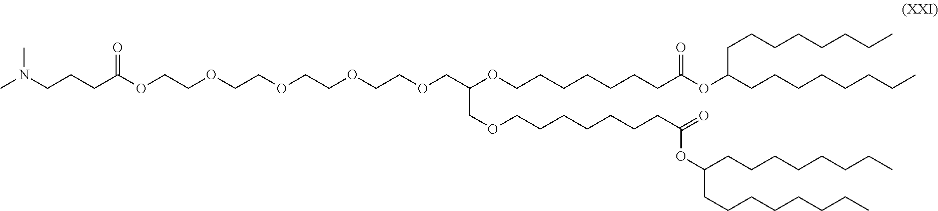 [SMILES: CCCCCCCCC(CCCCCCCC)OC(=O)CCCCCCCOCC(COCCOCCOCCOCCOC(=O)CCCN(C)C)OCCCCCCCC(=O)OC(CCCCCCCC)CCCCCCCC]

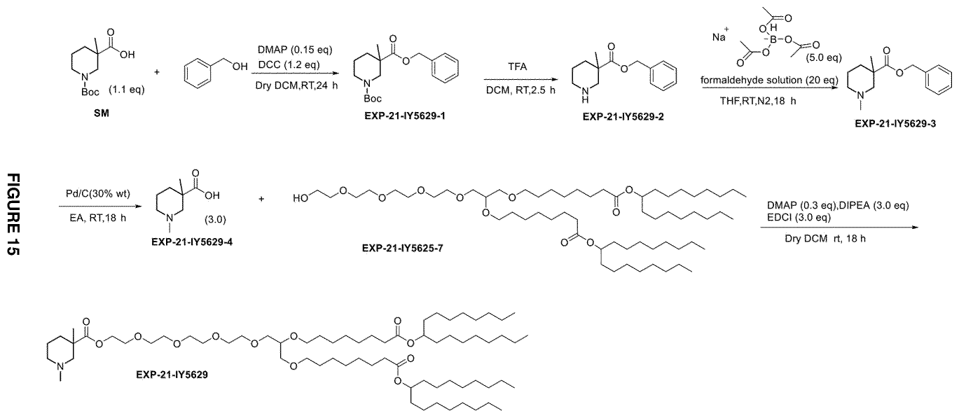

The compound XXI is prepared according to the schema of synthesis of FIG. 14.

### Synthesis of EXP-21-IY5625-1

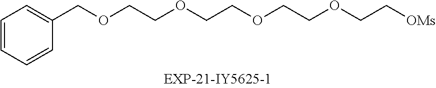 [SMILES: CS(=O)(=O)OCCOCCOCCOCCOCc1ccccc1]

To a mixture of 2-[2-[2-(2-benzyloxyethoxy) ethoxy]ethoxy] ethanol (50 g, 176 mmol) and Et₃N (35.6 g, 352 mmol) in DCM (400 mL) was added methanesulfonyl chloride (30.2 g, 264 mmol) slowly at 0° C. The mixture was stirred overnight at room temperature. CH₂Cl₂ (400 mL) were added to the solution, and the mixture was washed with diluted HCl (1M, 1000 mL). The mixture was shaken, the layers were separated, and the organic layer was collected. The organic layer was further washed with Water (1000 mL) and brine (1000 mL) and dried over Na₂SO₄. Solvent was then removed to give 2-[2-[2-(2-benzyloxyethoxy) ethoxy]ethoxy]ethyl methanesulfonate (64.1 g, 172 mmol, 97.5% yield) as an orange oil.

¹H NMR (400 MHZ, CDCl3) δ 7.37-7.27 (m, 5H), 4.56 (s, 2H), 4.40-4.33 (m, 2H), 3.78-3.72 (m, 2H), 3.69-3.60 (m, 12H), 3.06 (s, 3H).

### Synthesis of EXP-21-IY5625-2

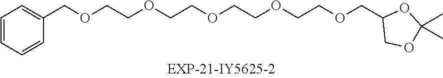 [SMILES: CC1(C)OCC(COCCOCCOCCOCCOCc2ccccc2)O1]

To the solution of (2,2-dimethyl-1,3-dioxolan-4-yl) methanol (24.6 g, 177 mmol) in THF (500 mL) was added NaH (14.1 g, 354 mmol) and the mixture was heated to 80° C. for 30 min. Then the reaction was cooled to room temperature and 2-[2-[2-(2-benzyloxyethoxy) ethoxy]ethoxy]ethyl methanesulfonate (64.1 g, 177 mmol) was added under nitrogen and the reaction was heated at 80° C. for 24 h. TLC indicated that the starting material was consumed. The reaction was quenched with water (300 mL) and extracted with ethyl acetate (600 mL). The aqueous layer was extracted with ethyl acetate (EA) (600 mL) again. The combined organic layers were washed with brine, dried over sodium sulfate, filtered and concentrated. The residue was purified through flash chromatography eluted with 20 to 50% ethyl acetate in petroleum ether to give 24-[2-[2-[2-(2-benzyloxyethoxy) ethoxy]ethoxy] ethoxymethyl]-2,2-dimethyl-1,3-dioxolane (48.635 g, 116 mmol, 65.6% yield) as light yellow oil.

LCMS MS (ESI) m/z=421.4 (M±23)⁺ at 2.154

1H NMR (500 MHZ, CDCl3) δ 7.36-7.26 (m, 5H), 4.57 (s, 2H), 4.32-4.22 (m, 1H), 4.07-4.01 (m, 1H), 3.75-3.70 (m, 1H), 3.69-3.60 (m, 16H), 3.59-3.55 (m, 1H), 3.51-3.47 (m, 1H), 1.42 (s, 3H), 1.35 (s, 3H).

### Synthesis of EXP-21-IY5625-3

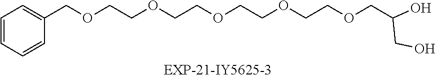 [SMILES: OCC(O)COCCOCCOCCOCCOCc1ccccc1]

The mixture of 4-[2-[2-[2-(2-benzyloxyethoxy) ethoxy]ethoxy] ethoxymethyl]-2,2-dimethyl-1,3-dioxolane (48.635 g, 122 mmol) in AcOH (200 mL) and H₂O (200 mL) was stirred at room temperature for 18 h. TLC (EA/PE 1/1, SM Rf: 0.5; product, Rf: 0.1) indicated that all the starting materials was consumed. The solvent was removed under vacuum and azeotroped with toluene several times. 2-[2-[2-(2-methylsulfonyloxyethoxy) ethoxy]ethoxy]ethyl methanesulfonate (43.7 g, 116 mmol, quant.) as light yellow oil was obtained which was used without further purification.

1H NMR (400 MHZ, CDCl3) δ 7.36-7.23 (m, 5H), 4.56 (s, 2H), 3.89-3.80 (m, 1H), 3.72-3.49 (m, 21H).

### Synthesis of EXP-21-IY5625-4

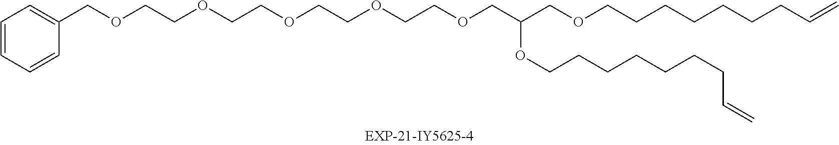 [SMILES: C=CCCCCCCCOCC(COCCOCCOCCOCCOCc1ccccc1)OCCCCCCCC=C]

To a solution of 3-[2-[2-[2-(2-benzyloxyethoxy) ethoxy]ethoxy] ethoxy]propane-1,2-diol (24 g, 60.3 mmol) in dry DMF (200 mL) under nitrogen was added NaH (9.64 g, 241 mmol) and the mixture was heated at 80° C. for 15 min. Then the reaction was cooled to room temperature and 9-bromonon-1-ene (31.9 g, 151 mmol) was added dropwise to this solution. The mixture was stirred at room temperature for 30 min and then at 80° C. for 18 h. TLC (EA/PE=1/1, Rf: 0.5) indicated that a new spot was formed. The reaction was quenched with water (50 mL) and then partitioned between ethyl acetate and water. The aqueous layer was extracted with ethyl acetate again. The combined organic layers were dried over sodium sulfate, filtered and concentrated. The residue was purified by flash chromatography eluted with 20% to 50% ethyl acetate in petroleum ether to give 2-[2-[2-[2-[2,3-bis (non-8-enoxy) propoxy] ethoxy]ethoxy] ethoxy]ethoxymethylbenzene (13.24 g, 20.7 mmol, 32.6% yield) as light yellow oil.

1H NMR (500 MHZ, CDCl3) δ 7.36-7.27 (m, 5H), 5.86-5.75 (m, 2H), 5.03-4.89 (m, 4H), 4.57 (s, 2H), 3.69-3.61 (m, 17H), 3.58-3.40 (m, 9H), 2.08-1.99 (m, 4H), 1.61-1.51 (m, 4H), 1.42-1.27 (m, 16H).

### Synthesis of EXP-21-IY5625-5

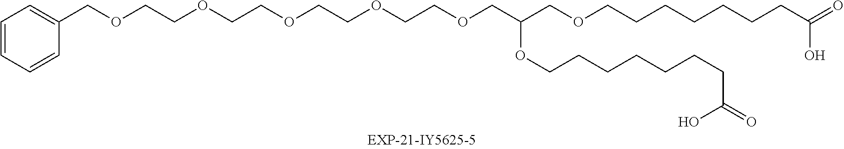 [SMILES: O=C(O)CCCCCCCOCC(COCCOCCOCCOCCOCc1ccccc1)OCCCCCCCC(=O)O]

To a solution of 2-[2-[2-[2-[2,3-bis (non-8-enoxy) propoxy] ethoxy]ethoxy] ethoxy]ethoxymethyl benzene (13.24 g, 20.7 mmol) in MeCN (120 mL), CCl₄ (120 mL) and water (120 mL) was added NalO₄ (35.5 g, 166 mmol) and RuCl₃ (935 mg, 4.15 mmol). The reaction mixture was stirred at room temperature for 18 h. LCMS indicated that the title compound was the major product along with partial mono-aldehyde product. The reaction was filtered and the filtrate was diluted with ethyl acetate (800 mL) and washed with 1N aq. HCl (400 mL). The organic layer was washed with Na2S₂O₃ solution and then dried over sodium sulfate, filtered and concentrated to give 8-[3-[2-[2-[2-(2-benzyloxyethoxy) ethoxy]ethoxy] ethoxy]-2-(7-carboxyheptoxy) propoxy] octanoic acid (13.3 g, 8.28 mmol, 39.9% yield) as pale green oil which was used without further purification.

¹H NMR (500 MHZ, CDCl3) δ 9.79-9.72 (m, 1H), 7.36-7.27 (m, 5H), 4.57 (s, 2H), 3.72-3.37 (m, 26H), 2.45-2.39 (m, 1H), 2.32 (t, J=7.3 Hz, 2H), 1.68-1.49 (m, 8H), 1.32 (s, 12H).

8-[3-[2-[2-[2-(2-benzyloxyethoxy) ethoxy]ethoxy] ethoxy]-2-(8-oxooctoxy) propoxy] octanoic acid (13.3 g, 12.7 mmol, 60% purity) was dissolved in t-BuOH: H₂O (3:1, 100 mL), containing NaH₂PO₄.2H₂O (7.64 g, 63.7 mmol), 2-methy-2-butene (60 mL) and sodium chlorite (7.2 g, 63.7 mmol). The reaction was stirred for 2 h at room temperature and LCMS indicated that the starting material was consumed. The reaction mixture was diluted with ethyl acetate. The aqueous layer was extracted with ethyl acetate.

The combined extract was dried over sodium sulfate. The residue was purified by flash chromatography eluted with 0% to 5% CH₃OH in DCM to give 8-[3-[2-[2-[2-(2-benzyloxyethoxy) ethoxy]ethoxy] ethoxy]-2-(7-carboxyheptoxy) propoxy] octanoic acid (6.65 g, 9.83 mmol, 77.2% yield) and partial oxidation product (2.78 g) as light yellow oil.

¹H NMR (500 MHZ, CDCl3) δ 7.37-7.27 (m, 5H), 4.57 (s, 2H), 3.69-3.61 (m, 16H), 3.58-3.40 (m, 10H), 2.38-2.25 (m, 4H), 1.68-1.48 (m, 8H), 1.34 (d, J=14.9 Hz, 12H).

### Synthesis of EXP-21-IY5625-6

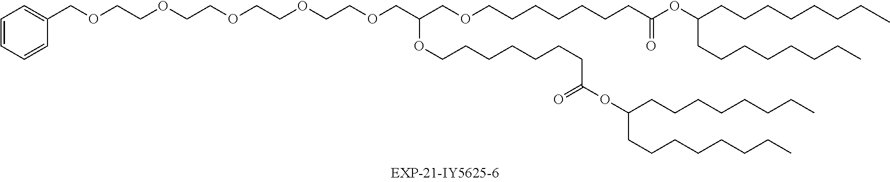 [SMILES: CCCCCCCCC(CCCCCCCC)OC(=O)CCCCCCCOCC(COCCOCCOCCOCCOCc1ccccc1)OCCCCCCCC(=O)OC(CCCCCCCC)CCCCCCCC]

A mixture of 8-[3-[2-[2-[2-(2-benzyloxyethoxy) ethoxy]ethoxy] ethoxy]-2-(7-carboxyheptoxy) propoxy] octanoic acid (6.65 g, 10.3 mmol), heptadecan-9-ol (7.96 g, 31 mmol), DMAP (506 mg, 4.14 mmol), EDCl HCl (5.16 g, 26.9 mmol) and DIEA (8.02 g, 62.1 mmol) in Dry DCM (100 mL). The mixture was stirred for 16 h at room temperature. The mixture was added DCM (500 mL) and washed with 1 N HCl, NaCl and concentrated. The residue was purified by flash column chromatography on silica gel eluting with CH₃OH in DCM (0 to 4%) to give 1-octylnonyl 8-[3-[2-[2-[2-(2-benzyloxyethoxy) ethoxy]ethoxy] ethoxy]-2-[8-(1-octylnonoxy)-8-oxo-octoxy] propoxy] octanoate (3.6 g, 3.05 mmol, 30.5% yield) as pale yellow oil.

1H NMR (400 MHZ, CDCl3) δ 7.36-7.27 (m, 5H), 4.90-4.81 (m, 2H), 4.57 (s, 2H), 3.71-3.61 (m, 16H), 3.59-3.39 (m, 9H), 2.27 (t, J=7.1 Hz, 4H), 1.62-1.46 (m, 16H), 1.28 (d, J=22.7 Hz, 60H), 0.91-0.84 (m, 12H).

### Synthesis of EXP-21-IY5625-7

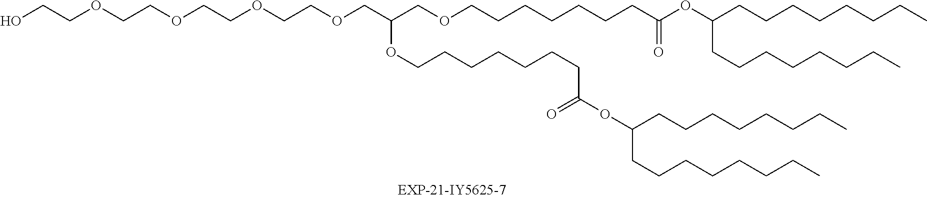 [SMILES: CCCCCCCCC(CCCCCCCC)OC(=O)CCCCCCCOCC(COCCOCCOCCOCCO)OCCCCCCCC(=O)OC(CCCCCCCC)CCCCCCCC]

To the solution of 1-octylnonyl 8-[3-[2-[2-[2-(2-benzyloxyethoxy) ethoxy]ethoxy] ethoxy]-2-[8-(1-octylnonoxy)-8-oxo-octoxy] propoxy] octanoate (3.6 g, 3.22 mmol) in ethyl acetate (50 mL) was added Pd/C (1.1 g, 30% wt/wt). The mixture was stirred at room temperature under hydrogen for 18 h. TLC (CH₃OH/DCM (3%)) indicated that the starting material was consumed. The reaction was filtered through celite and washed with ethyl acetate to give 1-octylnonyl 8-[3-[2-[2-[2-(2-hydroxy ethoxy) ethoxy]ethoxy] ethoxy]-2-[8-(1-octylnonoxy)-8-oxo-octoxy] propoxy] octanoate (3.187 g, 3.1 mmol, 96.3% yield) as colorless oil. 1H NMR (400 MHZ, CDCl3) δ 4.91-4.80 (m, 2H), 3.75-3.71 (m, 2H), 3.70-3.39 (m, 23H), 2.27 (t, J=7.5 Hz, 4H), 1.67-1.45 (m, 16H), 1.35-1.20 (m, 60H), 0.92-0.81 (m, 12H).

### Synthesis of the Compound XXI

To a solution of 1-octylnonyl 8-[3-[2-[2-[2-(2-hydroxyethoxy) ethoxy]ethoxy] ethoxy]-2-[8-(1-octylnonoxy)-8-oxo-octoxy] propoxy] octanoate (0.400 g, 0.398 mmol) and 5-(dimethylamino) pentanoic acid hydrochloride (0.201 g, 1.17 mmol) in dry dichloromethane (15 mL) was added DIPEA (0.301 g, 2.33 mmol) and DMAP (0.015 g, 0.117 mmol). Then added to EDCl (0.223 g, 1.17 mmol) at 0° C. The reaction was allowed to stir at room temperature for 18 h. The reaction was diluted with DCM and washed with saturated sodium bicarbonate. The organic layer was separated and washed with brine and dried over Na2SO4. The organic layer was filtered and evaporated under vacuum. The residue was purified by silica gel chromatography (0-5% CH₃OH in DCM (4%)) to obtain 1-octylnonyl 8-[3-[2-[2-[2-[2-[4-(dimethylamino) butanoyloxy] ethoxy]ethoxy] ethoxy]ethoxy]-2-[8-(1-octylnonoxy)-8-oxo-octoxy] propoxy] octanoate (0.2952 g, 0.251 mmol, 64.5% yield) as yellow oil.

LCMS MS (ESI) m/z=1143.0 (M+H)⁺ at 4.635 min

¹H NMR (500 MHZ, CDCl3) δ 4.90-4.80 (m, 2H), 4.25-4.21 (m, 2H), 3.71-3.67 (m, 2H), 3.64 (d, J=8.7 Hz, 12H), 3.59-3.39 (m, 9H), 2.45-2.34 (m, 4H), 2.34-2.22 (m, 10H), 1.88-1.81 (m, 2H), 1.61 (s, 4H), 1.57-1.47 (m, 12H), 1.34-1.23 (m, 60H), 0.91-0.85 (m, 12H).

### Example 17: Synthesis of 1-octylnonyl 8-[3-[2-[2-[2-[2-[3-(dimethylamino) propanoyloxy] ethoxy]ethoxy] ethoxy]ethoxy]-2-[8-(1-octylnonoxy)-8-oxo-octoxy] propoxy] octanoate (compound XXII)

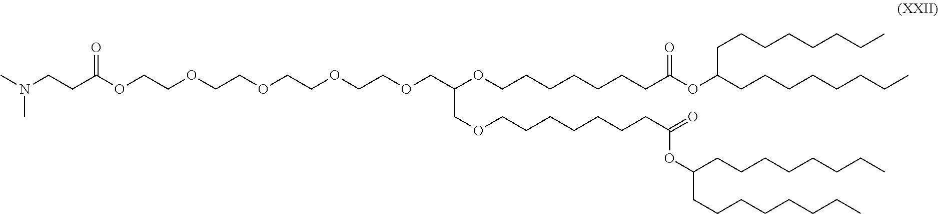 [SMILES: CCCCCCCCC(CCCCCCCC)OC(=O)CCCCCCCOCC(COCCOCCOCCOCCOC(=O)CCN(C)C)OCCCCCCCC(=O)OC(CCCCCCCC)CCCCCCCC]

The compound XXII is prepared as follow:

To a solution of 1-octylnonyl 8-[3-[2-[2-[2-(2-hydroxyethoxy) ethoxy]ethoxy] ethoxy]-2-[8-(1-octylnonoxy)-8-oxo-octoxy] propoxy] octanoate (0.500 g, 0.486 mmol) and 3-(dimethylamino) propanoic acid;hydrochloride (0.224 g, 1.46 mmol) in dry dichloromethane (15 mL) was added DIPEA (0.377 g, 2.91 mmol) and DMAP (0.018 g, 0.146 mmol). Then added to EDCl (0.279 g, 1.46 mmol) at OoC. The reaction was allowed to stir at room temperature for 18 h. The reaction was diluted with dichloromethane and washed with saturated sodium bicarbonate. The organic layer was separated, washed with brine and dried over Na2SO4. The organic layer was filtered and evaporated under vacuum. The residue was purified by silica gel chromatography (0-5% CH₃OH in DCM (4%)) to obtain target product (0.2991 g, 0.257 mmol, 52.9% yield) as yellow oil.

LCMS; Find peak MS (ESI) m/z=1129.1 (M+H)⁺ at 4.496 min

1H NMR (500 MHZ, CDCl3) δ 4.91-4.80 (m, 2H), 4.27-4.22 (m, 2H), 3.73-3.37 (m, 23H), 2.70-2.60 (m, 2H), 2.57-2.50 (m, 2H), 2.31-2.23 (m, 10H), 1.64-1.47 (m, 16H), 1.33-1.23 (m, 61H), 0.90-0.85 (m, 12H).

### Example 18: Synthesis of 2-[2-[2-[2-[2,3-bis [8-(1-octylnonoxy)-8-oxo-octoxy] propoxy] ethoxy]ethoxy] ethoxy]ethyl 1,3-dimethylpiperidine-3-carboxylate (compound XXIII)

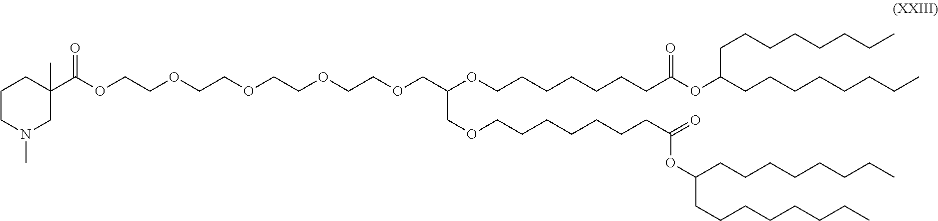 [SMILES: CCCCCCCCC(CCCCCCCC)OC(=O)CCCCCCCOCC(COCCOCCOCCOCCOC(=O)C1(C)CCCN(C)C1)OCCCCCCCC(=O)OC(CCCCCCCC)CCCCCCCC]

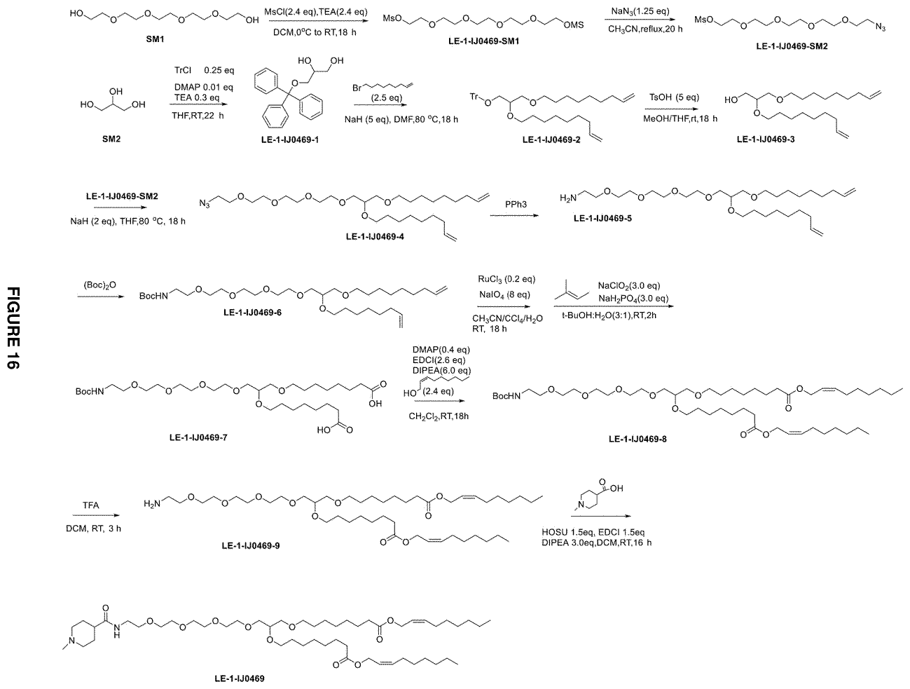

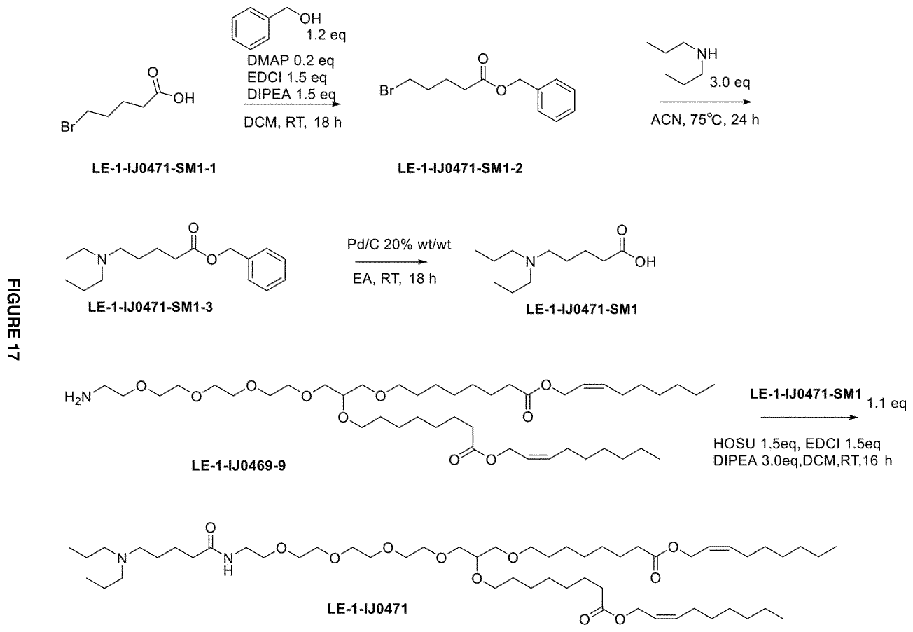

The compound XXIII is prepared according to the schema of synthesis of FIG. 15.

### Synthesis of EXP-21-IY5629-1

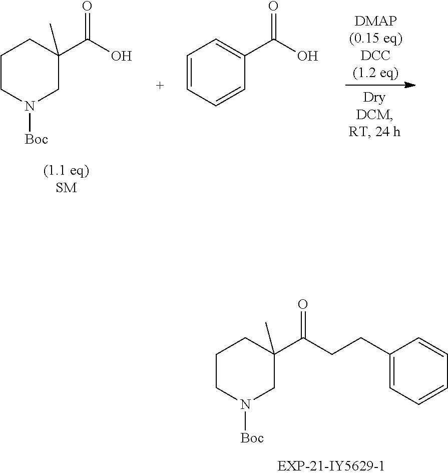 [SMILES: CC(C)(C)OC(=O)N1CCCC(C)(C(=O)O)C1 ; O=C(O)c1ccccc1 ; CC(C)(C)OC(=O)N1CCCC(C)(C(=O)CCc2ccccc2)C1]

To a solution of phenylmethanol (2.0 g, 18.5 mmol), 1-tert-butoxycarbonyl-3-methyl-piperidine-3-carboxylic acid (4.95 g, 20.3 mmol) and DMAP (340 mg, 2.77 mmol) were dissolved in Dry DCM (50 mL). A solution of DCC (4.58 g, 22.2 mmol) in Dry DCM (20 mL) was added dropwise then stirred for 24 hours at room temperature. A precipitate formed and was removed by filtration. The solvent was evaporated, and the resulting product was purified by column chromatography (PE in ethyl acetate (EA) (0-10%)) to give 03-benzyl O1-tert-butyl 3-methylpiperidine-1,3-dicarboxylate (3.3 g, 9.89 mmol, 53.4% yield) as colorless oil.

LCMS; Find peak MS (ESI) m/z=356.2 (M+Na)⁺ at 2.25 min.

¹H NMR (400 MHZ, CDCl3) δ 7.41-7.28 (m, 5H), 5.20-5.05 (m, 2H), 3.82 (d, J=13.3 Hz, 1H), 3.44-3.16 (m, 3H), 2.09-1.98 (m, 1H), 1.59-1.39 (m, 12H), 1.19 (s, 3H).

### Synthesis of EXP-21-IY5629-2

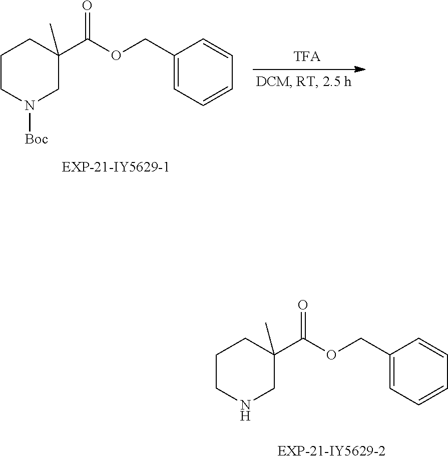 [SMILES: CC(C)(C)OC(=O)N1CCCC(C)(C(=O)OCc2ccccc2)C1 ; CC1(C(=O)OCc2ccccc2)CCCNC1]

03-benzyl O1-tert-butyl 3-methylpiperidine-1,3-dicarboxylate (3.3 g, 9.9 mmol) was dissolved in 30 mL DCM and then added TFA (12 mL). The mixture was stirred at room temperature 18 h. The solvent was evaporated (50 mL DCM*2) and then dissolved in DCM (100 mL) and washed with saturated NaHCO₃ (30 mL), organic layer was separated and dried over Na2SO4, filtrated and solvent was removed under reduced to give benzyl 3-methylpiperidine-3-carboxylate (1.906 g, 7.76 mmol, 78.4% yield) as yellow oil.

1H NMR (500 MHZ, CDCl3) δ 7.40-7.29 (m, 5H), 5.20-5.12 (m, 2H), 3.34 (d, J=12.9 Hz, 1H), 2.91 (dd, J=12.9, 3.9 Hz, 1H), 2.62-2.54 (m, 1H), 2.43 (d, J=12.9 Hz, 1H), 2.20 (dd, J=7.8, 5.6 Hz, 1H), 1.53 (dt, J=13.1, 4.0 Hz, 1H), 1.44-1.33 (m, 2H), 1.12 (s, 3H).

### Synthesis of EXP-21-IY5629-3

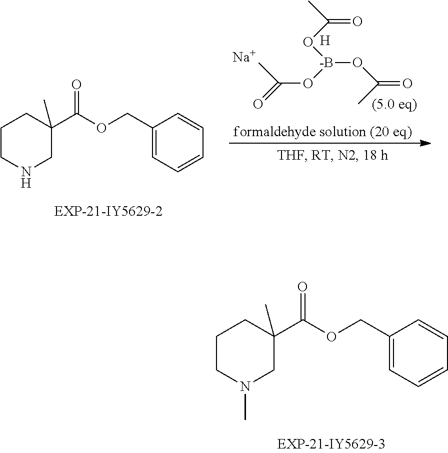

To a solution of benzyl 3-methylpiperidine-3-carboxylate (1.91 g, 8.17 mmol) in THF (80 mL) was added formaldehyde solution (13 g of 37% solution in water) at RT. The resulting mixture was stirred for 30 min before introducing sodium triacetoxyborohydride (5.0 eq, 40.8 mmol, 8.66 g). The mixture was stirred at room temperature 18 h under N2. The reaction mixture was concentrated. The reside was taken in EA (50 mL*2) and washed with NaHCO₃ solution. After dried over Na2SO4, the solution was concentrated and purified by flash dry column chromatography on silica gel (0-5% CH₃OH in DCM) to give benzyl 1,3-dimethylpiperidine-3-carboxylate (1.57 g, 6.22 mmol, 76.1% yield) as yellow oil.

1H NMR (400 MHZ, CDCl3) δ 7.40-7.27 (m, 5H), 5.16 (s, 2H), 2.99 (d, J=10.6 Hz, 1H), 2.56 (s, 1H), 2.23 (s, 3H), 2.13-1.86 (m, 3H), 1.77-1.66 (m, 1H), 1.61-1.55 (m, 1H), 1.17 (s, 4H).

LCMS; Find peak MS (ESI) m/z=248.21 (M+H)⁺ at 1.370 min

### Synthesis of EXP-21-IY5629-4

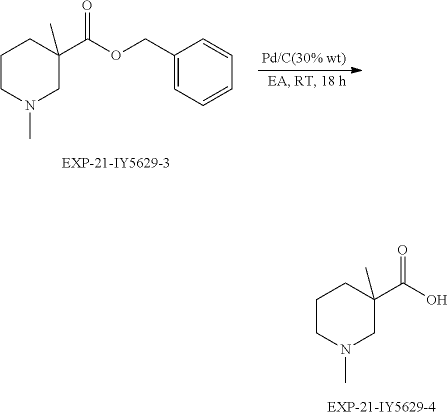 [SMILES: CN1CCCC(C)(C(=O)OCc2ccccc2)C1 ; CN1CCCC(C)(C(=O)O)C1]

To the solution of benzyl 1,3-dimethylpiperidine-3-carboxylate (1.57 g, 6.35 mmol) in ethyl acetate (20 mL) was added Pd/C (500 mg, 30% wt/wt). The mixture was stirred at room temperature under hydrogen for 18 h. TLC (CH₃OH/DCM (3%)) indicated that the starting material was consumed. The reaction was filtered through celite and washed with ethyl acetate to give 1-octylnonyl 1,3-dimethylpiperidine-3-carboxylic acid (0.888 g, 5.65 mmol, 89.0% yield) as colorless oil.

1H NMR (400 MHZ, CDCl3) δ 3.41 (d, J=11.5 Hz, 1H), 3.34-3.23 (m, 1H), 2.61 (s, 3H), 2.30-2.21 (m, 1H), 2.21-1.99 (m, 3H), 1.75-1.65 (m, 1H), 1.19-1.06 (m, 4H).

LCMSA; Find peak MS (ESI) m/z=158.2 (M+H)⁺ at 0.369 min

### Synthesis of Compound XXIII

To a solution of 1-octylnonyl 8-[3-[2-[2-[2-(2-hydroxyethoxy) ethoxy]ethoxy] ethoxy]-2-[8-(1-octyl nonoxy)-8-oxo-octoxy] propoxy] octanoate (0.700 g, 0.68 mmol) and 1,3-dimethylpiperidine-3-carboxylic acid (0.321 g, 2.04 mmol) in dry dichloromethane (15 mL) was added DIPEA (0.264 g, 2.04 mmol) and DMAP (0.025 g, 0.204 mmol). Then added to EDCl (0.391 g, 2.04 mmol) at 0° C. The reaction was allowed to stir at room temperature for 18 h. The reaction was diluted with dichloromethane and washed with saturated sodium bicarbonate. The organic layer was separated, washed with brine and dried over Na2SO4. The organic layer was filtered and evaporated under vacuum. The residue was purified by silica gel chromatography (0-5% CH₃OH in DCM (4%)) to obtain 2-[2-[2-[2-[2,3-bis [8-(1-octyl nonoxy)-8-oxo-octoxy] propoxy] ethoxy]ethoxy] ethoxy]ethyl 1,3-dimethylpiperidine-3-carboxylate (0.5683 g, 0.467 mmol, 68.7% yield) as colorless oil.

LCMS; Find peak MS (ESI) m/z=1169.1 (M+H)⁺ at 4.41 min

¹H NMR (500 MHZ, CDCl3) δ 4.90-4.82 (m, 2H), 4.33-4.19 (m, 2H), 3.72-3.67 (m, 2H), 3.66-3.60 (m, 12H), 3.58-3.39 (m, 9H), 2.96 (s, 1H), 2.55 (s, 1H), 2.30-2.25 (m, 4H), 2.22 (s, 3H), 2.11-1.81 (m, 3H), 1.62-1.48 (m, 16H), 1.34-1.22 (m, 62H), 1.17 (s, 4H), 0.90-0.85 (m, 12H).

### Example 19: Synthesis of [(Z)-non-2-enyl] 8-[3-[2-[2-[2-[2-[5-(dipropylamino) pentanoylamino]ethoxy]ethoxy] ethoxy]ethoxy]-2-[8-[(Z)-non-2-enoxy]-8-oxooctoxy] propoxy] octanoate (compound XXVII)

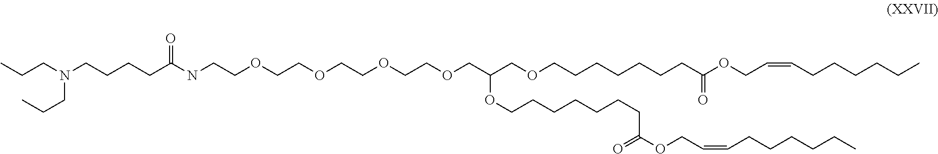 [SMILES: CCCCCC/C=C\COC(=O)CCCCCCCOCC(COCCOCCOCCOCCNC(=O)CCCCN(CCC)CCC)OCCCCCCCC(=O)OC/C=C\CCCCCC]

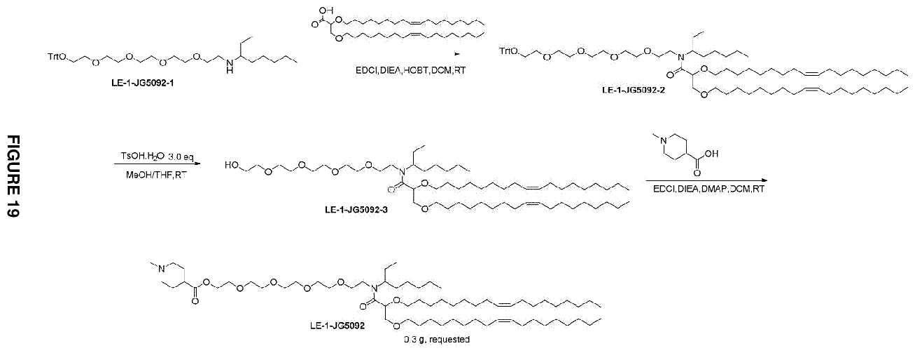

The compound XXVII is prepared according to the schema of synthesis of FIG. 17.

### Synthesis of EXP-21-IJ0471-SM1-2

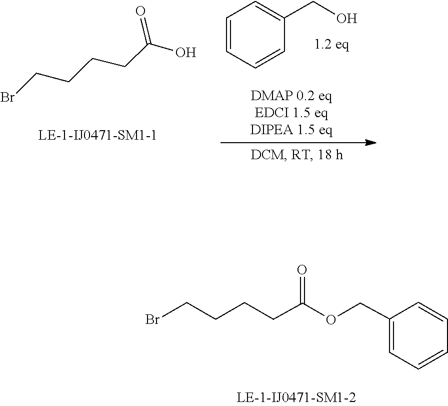

To the solution of Phenylmethanol (1.43 g, 0.0133 mol) and 5-bromopentanoic acid (2.0 g, 0.011 mol) in dry dichloromethane (20 mL) were added DIPEA (2.14 g, 0.0166 mol), DMAP (0.270 g, 2.21 mmol) and followed by EDCl (3.18 g, 0.0166 mol) portion wise under ice bath. The mixture was stirred at room temperature for 18 h. The reaction was diluted with dichloromethane and washed with brine. The organic layer was dried over sodium sulfate, filtered, and concentrated. The residue was purified by flash chromatography eluted with 0% to 10% (9%) ethyl acetate in petroleum ether to give benzyl 5-bromopentanoate (0.675 g, 23.2% yield) as colorless oil.

1H NMR (400 MHZ, CDCl3) δ 7.45-7.27 (m, 5H), 5.12 (s, 2H), 3.57-3.36 (m, 2H), 2.40 (t, J=7.2 Hz, 2H), 1.94-1.76 (m, 4H).

### Synthesis of LE-1J04771-SM1-3

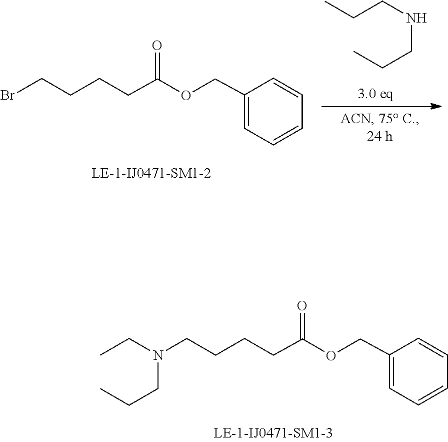

benzyl 5-bromopentanoate (695 mg, 2.56 mmol) and N-propylpropan-1-amine (0.778 g, 7.69 mmol) in 20 mL acetonitrile was stirred at 75° C. for 24 h. The reaction was cooled to room temperature and solvents were evaporated under vacuum. The residue was taken-up in ethyl acetate and saturated sodium bicarbonate. The organic layer was separated, dried over Na2SO4, and evaporated under vacuum. The residue was purified by silica gel chromatography (19-25% (20%) EA in PE) to obtain benzyl 5-(dipropylamino) pentanoate (0.48 g, 64.3 yield %) as colorless oil.

1H NMR (400 MHZ, CDCl3) δ 7.41-7.28 (m, 5H), 5.11 (s, 2H), 2.44-2.30 (m, 8H), 1.70-1.60 (m, 2H), 1.51-1.37 (m, 6H), 0.86 (t, J=7.4 Hz, 6H).

### Synthesis of LE-1J04771-SM1

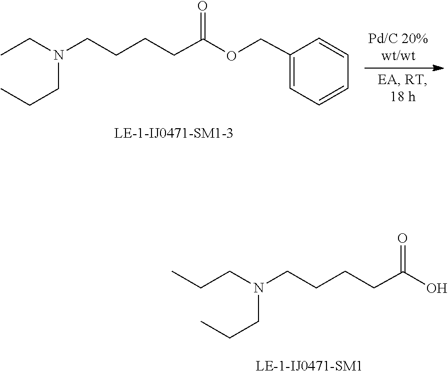 [SMILES: CCCN(CC)CCCCC(=O)OCc1ccccc1 ; CCCN(CCC)CCCCC(=O)O]

A solution of benzyl 5-(dipropylamino) pentanoate (0.42 g, 1.44 mmol) in EtOAc (10 mL) was purged for 10 minutes with N2 followed by addition of Pd/C (20% wt/wt, 0.1 g) and the reaction continued purging with N2. The reaction was next evacuated under vacuum and backfilled with H_{2 3} times. The reaction was stirred overnight at room temperature under an atmosphere of H₂. TLC indicated that the reaction was finished. The slurry was filtered through celite and washed with EtOAc several times. The combined organics were concentrated under vacuum to give 5-(dipropylamino) pentanoic acid (0.27 g, 93.1% yield) as white solid.

1H NMR (400 MHZ, CDCl3) δ 5.55 (s, 1H), 2.94-2.78 (m, 6H), 2.31-2.21 (m, 2H), 1.78-1.55 (m, 8H), 0.95 (t, J=7.4 Hz, 6H).

### Synthesis of Compound XXVII

A mixture of 5-(dipropylamino) pentanoic acid (0.138 g, 0.65 mmol), EDC HCl (0.144 g, 0.75 mmol), N-Hydroxysuccinimide (0.086 g, 0.75 mmol) in dry DCM (15 mL) was stirred for 2 h at rt. Then [(Z)-non-2-enyl] 8-[3-[2-[2-[2-(2-aminoethoxy) ethoxy]ethoxy] ethoxy]-2-[8-[(Z)-non-2-enoxy]-8-oxo-octoxy] propoxy] octanoate (0.4 g, 0.5 mmol) and DIEA (0.194 g, 1.5 mmol) were added. The mixture was stirred for 16 h at rt. TLC indicated that the product and the starting material were almost one spot. The mixture was diluted with dichloromethane (50 mL) and washed with water and brine, dried over Na2SO4 and concentrated. The residue was purified by column chromatography on silica gel eluted with MeOH in DCM (5-15%) to afford [(Z)-non-2-enyl] 8-[3-[2-[2-[2-[2-[5-(dipropylamino) pentanoylamino]ethoxy]ethoxy] ethoxy]ethoxy]-2-[8-[(Z)-non-2-enoxy]-8-oxo-octoxy] propoxy] octanoate (0.309 g, 0.314 mmol, 62.9% yield) as light yellow oil.

1H NMR (400 MHZ, CDCl3) δ 6.57 (s, 1H), 5.69-5.47 (m, 4H), 4.62 (d, J=6.8 Hz, 4H), 3.70-3.37 (m, 26H), 2.92-2.65 (m, 6H), 2.33-2.22 (m, 6H), 2.10 (q, J=7.0 Hz, 4H), 1.73-1.50 (m, 14H), 1.41-1.20 (m, 31H), 0.96 (t, J=7.3 Hz, 6H), 0.88 (t, J=6.8 Hz, 6H).

-LCMS; Ms (M+H): 983.8

### Example 20: Synthesis of [(Z)-non-2-enyl] 8-[3-[2-[2-[2-[2-[5-(dipropylamino) pentanoyloxy] ethoxy]ethoxy] ethoxy]ethoxy]-2-[8-[(Z)-non-2-enoxy]-8-oxo-octoxy] propoxy] octanoate (compound XXVIII)

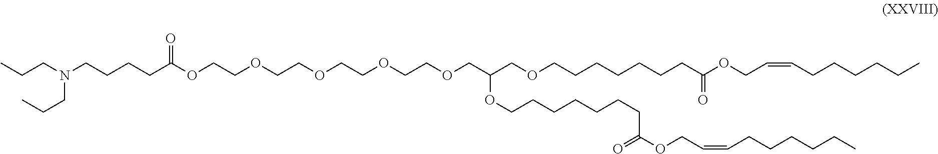 [SMILES: CCCCCC/C=C\COC(=O)CCCCCCCOCC(COCCOCCOCCOCCOC(=O)CCCCN(CCC)CCC)OCCCCCCCC(=O)OC/C=C\CCCCCC]

The compound XXVIII is prepared according to the schema of synthesis of FIG. 18.

### Synthesis of SM1-2

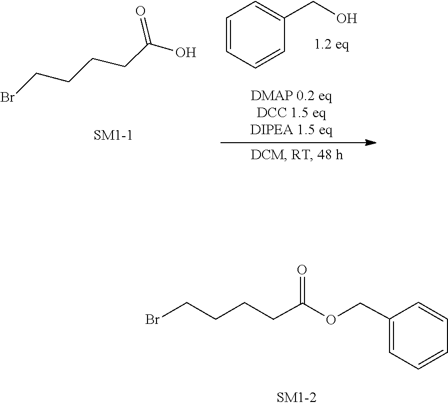

5-Bromopentanoic acid (13.20 g, 72.97 mmol), benzyl alcohol (5.43 g, 66.34 mmol) and N,N-dimethyl-4-aminopyiridine (DMAP) (1.62 g, 10.04 mmol) were dissolved in dry CH₂Cl₂ (200 mL). A solution of N,N′-dicyclohexylcarbodiimide (DCC) (12.43 g, 79.66 mmol) in dry CH₂Cl₂ (40 mL) was added dropwise and the mixture was stirred at room temperature for 24 hours. The white precipitate of N,N′-dicyclohexylurea was filtered off from the solution. The solvent was evaporated, and the resulting product was purified by column chromatography (silica gel, 0% to 10% (9%) ethyl acetate in petroleum ether to give benzyl 5-bromopentanoate (0.675 g, 23.2% yield) as colorless oil.

1H NMR (500 MHZ, CDCl3) δ 7.40-7.30 (m, 5H), 5.12 (s, 2H), 3.40 (t, J=6.5 Hz, 2H), 2.40 (t, J=7.2 Hz, 2H), 1.93-1.76 (m, 4H).

### Synthesis of SM1-3

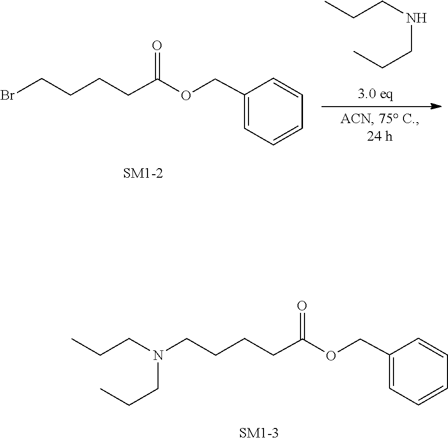

Benzyl 5-bromopentanoate (2.57 g, 9.48 mmol) and N-propylpropan-1-amine (2.88 g, 28.4 mmol) in 40 mL acetonitrile was stirred at 75° C. for 24 h. The reaction was cooled to room temperature and solvents were evaporated under vacuum. The residue was taken-up in ethyl acetate and saturated sodium bicarbonate. The organic layer was separated, dried over Na2SO4 and concentrated. The residue was purified by silica gel chromatography (0-25% (20%) EA in PE) to obtain benzyl 5-(dipropylamino) pentanoate (2.17 g, 78.6% yield) as colorless oil.

1H NMR (400 MHZ, CDCl3) δ 7.43-7.27 (m, 5H), 5.11 (s, 2H), 2.45-2.28 (m, 8H), 1.71-1.59 (m, 2H), 1.51-1.35 (m, 6H), 0.86 (t, J=7.4 Hz, 6H).

### Synthesis of SM1

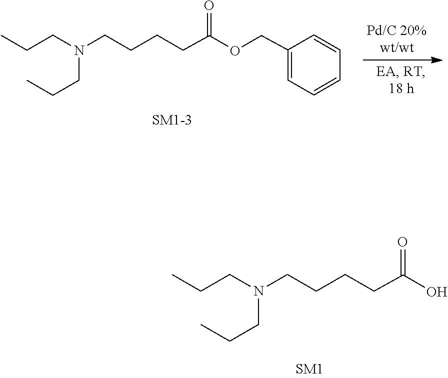 [SMILES: CCCN(CCC)CCCCC(=O)OCc1ccccc1 ; CCCN(CCC)CCCCC(=O)O]

A solution of benzyl 5-(dipropylamino) pentanoate (2.17 g, 7.45 mmol) in EtOAc (20 mL) was purged for 10 minutes with N2 followed by addition of Pd/C (20% wt/wt, 0.5 g) and the reaction continued purging with N2. The reaction was next evacuated under vacuum and backfilled with H_{2 3} times. The reaction was next stirred overnight at room temperature under an atmosphere of H₂. TLC indicated that the reaction was finished. The slurry was filtered through celite and the celite was rinsed with EtOAc several times. The combined organics were next concentrated under vacuum to give 5-(dipropylamino) pentanoic acid (1.46 g, 97.4% yield) as colorless oil.

¹H NMR (400 MHZ, CDCl3) δ 10.87 (s, 1H), 2.93-2.74 (m, 6H), 2.30-2.21 (m, 2H), 1.76-1.55 (m, 8H), 0.94 (t, J=7.3 Hz, 6H).

### Synthesis of the Compound XXVIII

To the solution of [(Z)-non-2-enyl] 8-[3-[2-[2-[2-(2-hydroxyethoxy) ethoxy]ethoxy] ethoxy]-2-[8-[(Z)-non-2-enoxy]-8-oxo-octoxy] propoxy] octanoate (0.4 g, 0.50 mmol) and 5-(dipropylamino) pentanoic acid (0.20 g, 1.00 mmol) in dry dichloromethane (15 mL) were added DIPEA (0.08 g, 0.60 mmol), DMAP (0.006 g, 0.05 mmol) and followed by EDCl (0.12 g, 0.60 mmol). The mixture was stirred at room temperature for 18 h. The reaction was diluted with dichloromethane and washed with brine. The organic layer was dried over sodium sulfate, filtered and concentrated. The residue was purified by flash chromatography eluted with 0% to 10% (6%) methanol in DCM to give 6-[2-[6-(2-hexyldecanoyloxy) hexoxy]-3-[2-[2-[2-[2-[2-[2-[2-[2-(2-methoxyethylamino) acetyl] oxyethylamino] acetyl] oxyethoxy]ethoxy] ethoxy]ethoxy]ethyl-octyl-amino]-3-oxo-propoxy] hexyl 2-hexyldecanoate (325 mg, 66.1% yield) as colorless oil.

¹H NMR (500 MHZ, CDCl3) δ 5.68-5.60 (m, 2H), 5.56-5.48 (m, 2H), 4.62 (d, J=6.8 Hz, 4H), 4.25-4.21 (m, 2H), 3.72-3.68 (m, 2H), 3.66-3.62 (m, 12H), 3.57-3.39 (m, 9H), 2.72 (s, 5H), 2.39 (t, J=7.0 Hz, 2H), 2.33-2.27 (m, 4H), 2.10 (q, J=6.8 Hz, 4H), 1.80-1.48 (m, 19H), 1.33-1.24 (m, 26H), 0.95 (t, J=7.3 Hz, 6H), 0.88 (t, J=7.0 Hz, 6H).

### Example 21: Synthesis of 2-[2-[2-[2-[2-[2,3-bis [(Z)-octadec-9-enoxy] propanoyl-(1-ethylhexyl) amino]ethoxy]ethoxy] ethoxy]ethoxy]ethyl 1-methylpiperidine-4-carboxylate (compound XXIX)

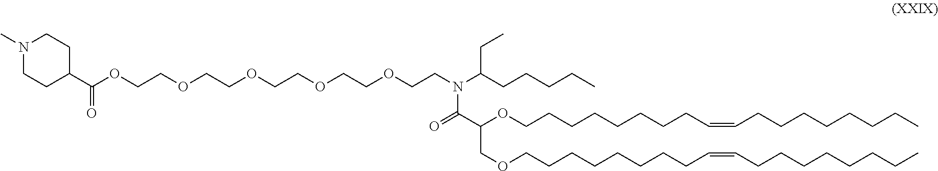 [SMILES: CCCCCCCC/C=C\CCCCCCCCOCC(OCCCCCCCC/C=C\CCCCCCCC)C(=O)N(CCOCCOCCOCCOCCOC(=O)C1CCN(C)CC1)C(CC)CCCCC]

The compound XXIX is prepared according to the schema of synthesis submitted in FIG. 19.

### Synthesis of the intermediate LE-1-JG5082-1

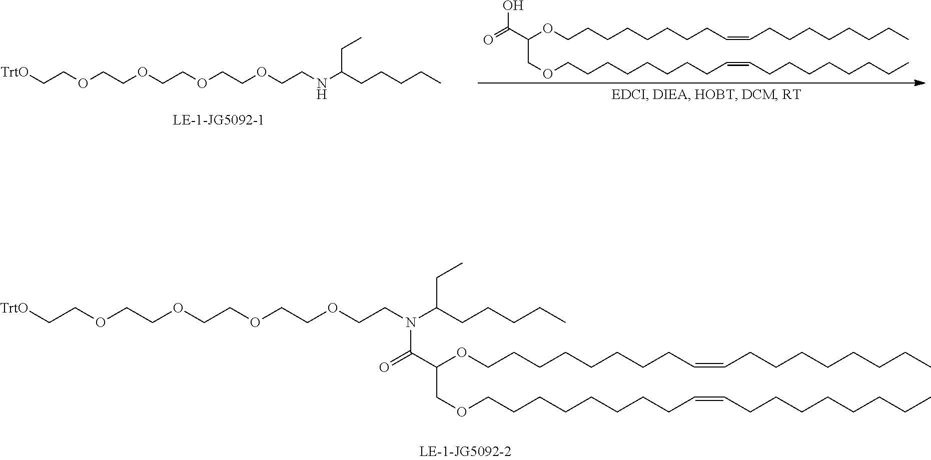

To a solution of N-[2-[2-[2-[2-(2-trityloxyethoxy) ethoxy]ethoxy] ethoxy] ethyl] octan-3-amine (0.5 g, 0.84 mmol) in DCM (50 ml) were added 2,3-bis [(Z)-octadec-9-enoxy] propanoic acid (0.76 g, 1.27 mmol), 3-(ethyliminomethyleneamino)-N,N-dimethyl-propan-1-amine;hydrochloride (320 mg, 1.96 mmol), 1-hydroxybenzotriazole (HOBT) (171 mg, 1.27 mmol) and N-ethyl-N-isopropyl-propan-2-amine (324 mg, 2.53 mmol). The mixture was stirred at 25° C. for 18 hr. Then the mixture was concentrated and dealt with EA (50 ml), washed with water (50 ml×2), brine (50 ml) and dried over Na2SO4. The organic was concentrated and purified by flash chromatography column (0-30% EA in PE) to give 3N-(1-ethylhexyl)-2,3-bis [(Z)-octadec-9-enoxy]-N-[2-[2-[2-[2-(2-trityloxyethoxy) ethoxy]ethoxy] ethoxy]ethyl]propanamide (0.59 g, yield 59.1%) as a colorless oil.

¹H NMR (400 MHZ, CDCl3) δ 7.46 (d, J=7.6 Hz, 5H), 7.27-7.14 (m, 13H), 5.34 (s, 3H), 3.83-3.13 (m, 24H), 2.06-1.90 (m, 7H), 1.51-1.41 (m, 3H), 1.26 (s, 50H), 0.98-0.74 (m, 12H).

### Synthesis of the Intermediate LE-1-JG5092-3

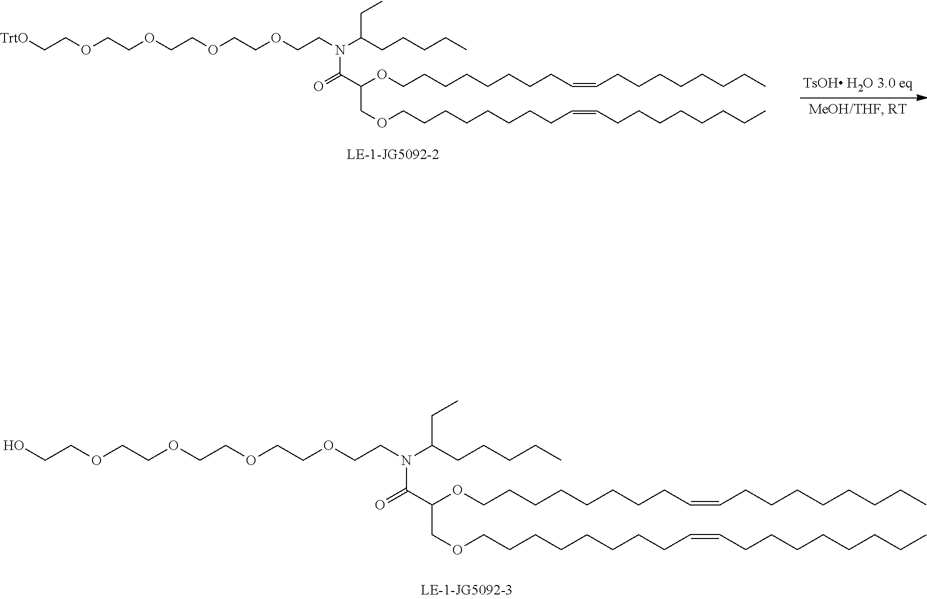

A mixture of N-(1-ethylhexyl)-2,3-bis [(Z)-octadec-9-enoxy]-N-[2-[2-[2-[2-(2-trityloxyethoxy) ethoxy]ethoxy] ethoxy]ethyl]propanamide (0.59 g, 0.5 mmol) and 4-methylbenzenesulfonic acid hydrate (0.285 g, 1.5 mmol) in MeOH (5 mL). The mixture was stirred for 16 h at room temperature. The mixture was concentrated and the residue was purified by column chromatography on silica gel eluting with 0% ˜10% MeOH in DCM to afford N-(1-ethylhexyl)-N-[2-[2-[2-[2-(2-hydroxyethoxy) ethoxy]ethoxy] ethoxy]ethyl]-2,3-bis [(Z)-octadec-9-enoxy] propanamide (0.4 g, 85.3%) as yellow oil.

### Synthesis of the Compound XXIX

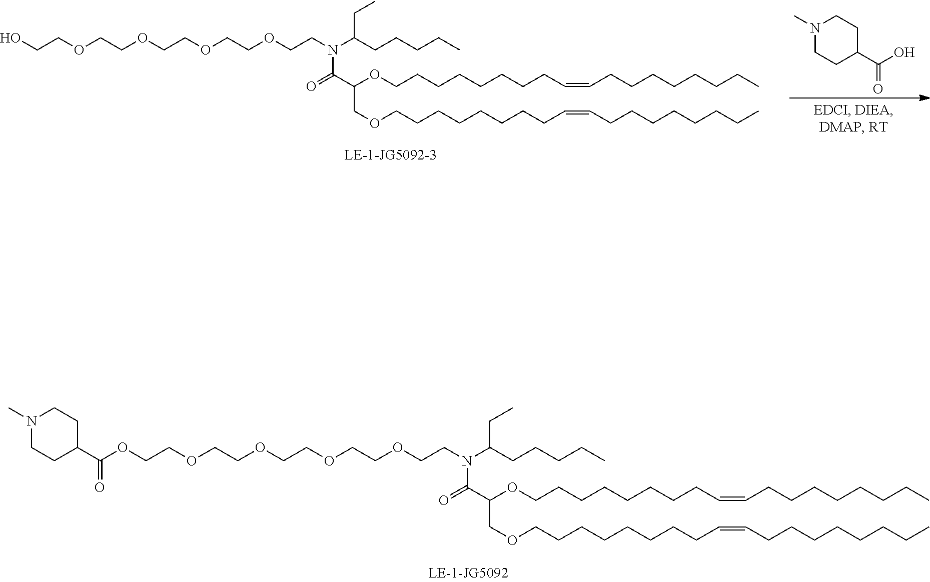

To a solution of N-(1-ethylhexyl)-N-[2-[2-[2-[2-(2-hydroxyethoxy) ethoxy] ethoxy]ethoxy]ethyl]-2,3-bis [(Z)-octadec-9-enoxy] propanamide (0.4 g, 0.42 mmol) and 1-methylpiperidine-4-carboxylic acid (0.183 g, 1.28 mmol) in dry dichloromethane (5 mL) was added DIPEA (0.165 g, 1.28 mmol), DMAP (0.052 g, 0.42 mmol) and followed by EDCl (0.163 g, 0.85 mmol). The mixture was stirred at room temperature for 18 h. The reaction was diluted with dichloromethane (50 mL) and washed with brine (25 mL). The organic layer was dried over sodium sulfate, filtered and concentrated. The residue was purified by flash chromatography eluted with 0% to 10% (7%) MeOH in DCM to give 2-[2-[2-[2-[2-[2,3-bis [(Z)-octadec-9-enoxy] propanoyl-(1-ethylhexyl) amino]ethoxy]ethoxy] ethoxy]ethoxy]ethyl 1-methylpiperidine-4-carboxylate (0.4 g, 88.2% yield) as light yellow oil.

¹H NMR (400 MHZ, CDCl3) δ 5.33 (dd, J=13.5, 8.3 Hz, 3H), 4.43 (d, J=14.0 Hz, 1H), 4.30-4.17 (m, 2H), 3.80-3.23 (m, 23H), 2.83 (s, 2H), 2.30 (s, 4H), 1.95 (dd, J=41.9, 35.3 Hz, 13H), 1.57-1.43 (m, 10H), 1.23 (d, J=28.9 Hz, 49H), 0.96-0.75 (m, 12H).

### Example 22: synthesis of 2-[2-[2-[2-[2-[2,3-bis [6-(1-hexylnonoxy)-6-oxo-hexoxy] propanoyl-octyl-amino]ethoxy]ethoxy] ethoxy]ethoxy]ethyl 1-methylpiperidine-4-carboxylate (compound XXX)

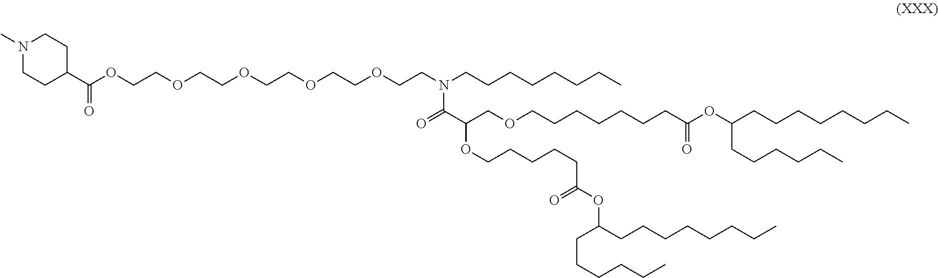 [SMILES: CCCCCCCCC(CCCCCC)OC(=O)CCCCCCCOCC(OCCCCCC(=O)OC(CCCCCC)CCCCCCCC)C(=O)N(CCCCCCCC)CCOCCOCCOCCOCCOC(=O)C1CCN(C)CC1]

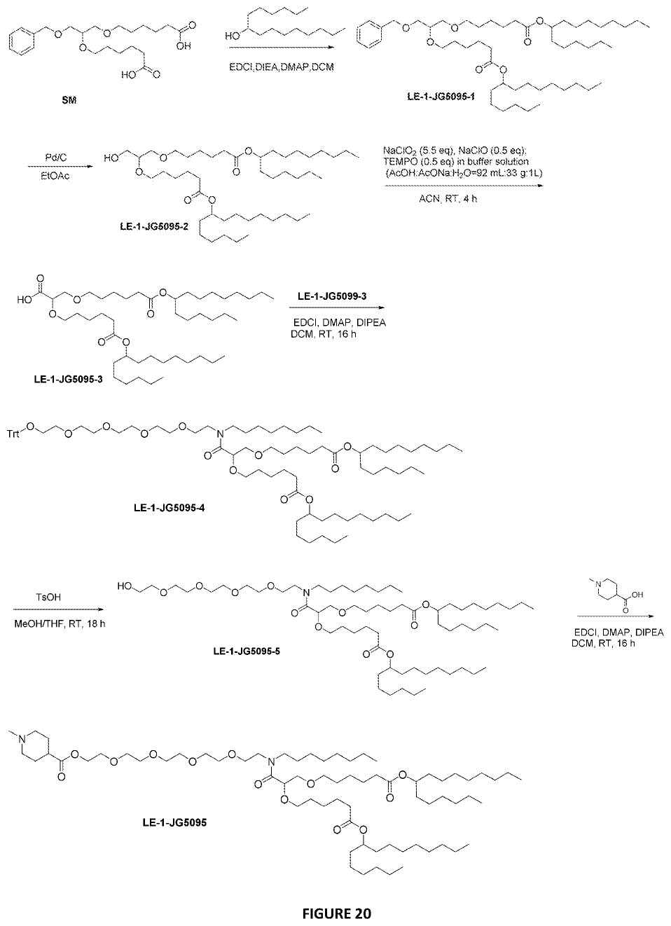

The compound XXX is prepared according to the schema of synthesis of FIG. 20.

### Synthesis of the intermediate LE-1-JG5095-1

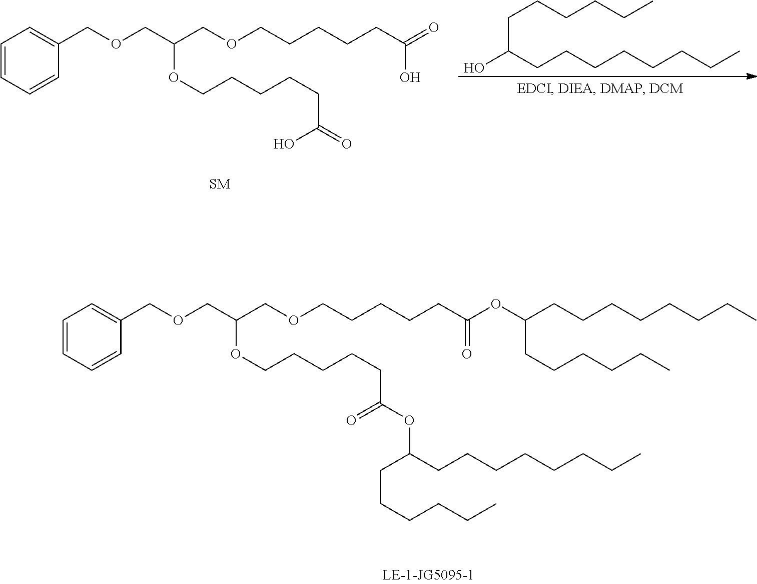 [SMILES: O=C(O)CCCCCOCC(COCc1ccccc1)OCCCCCC(=O)O ; CCCCCCCCC(O)CCCCCC ; CCCCCCCCC(CCCCCC)OC(=O)CCCCCOCC(COCc1ccccc1)OCCCCCC(=O)OC(CCCCCC)CCCCCCCC]

To a solution of 6-[3-benzyloxy-2-(5-carboxypentoxy) propoxy] hexanoic acid (1 g, 2.44 mmol) in DCM (50 ml) was added pentadecan-7-ol (1.67 g, 7.31 mmol), N-ethyl-N-isopropyl-propan-2-amine (0.945 g, 7.31 mmol), N,N-dimethylpyridin-4-amine (0.06 g, 0.49 mmol), 3-(ethyliminomethyleneamino)-N,N-dimethyl-propan-1-amine; hydrochloride (1.4 g, 7.31 mmol). The mixture was stirred at room temperature for 18 hr. The mixture was added DCM (100 mL) and washed with 0.5 N HCl (50 ml), NaHCO₃ (50 ml), NaCl (50 ml). The organic was concentrated and the residue was purified by flash column chromatography on silica gel eluting with 1:2 ethyl acetate/petroleum ether to give 1-hexylnonyl 6-[3-benzyloxy-2-[6-(1-hexylnonoxy)-6-oxo-hexoxy] propoxy] hexanoate (2.2 g, 87%, purity: 80%) as colorless oil.

1H NMR (400 MHZ, CDCl3) δ 7.38-7.27 (m, 5H), 4.86 (p, J=6.1 Hz, 2H), 4.55 (s, 2H), 3.67-3.39 (m, 11H), 2.27 (t, J=7.6 Hz, 4H), 1.72-1.15 (m, 108H), 0.95-0.81 (m, 22H).

### Synthesis of the Intermediate LE-1-JG5095-2

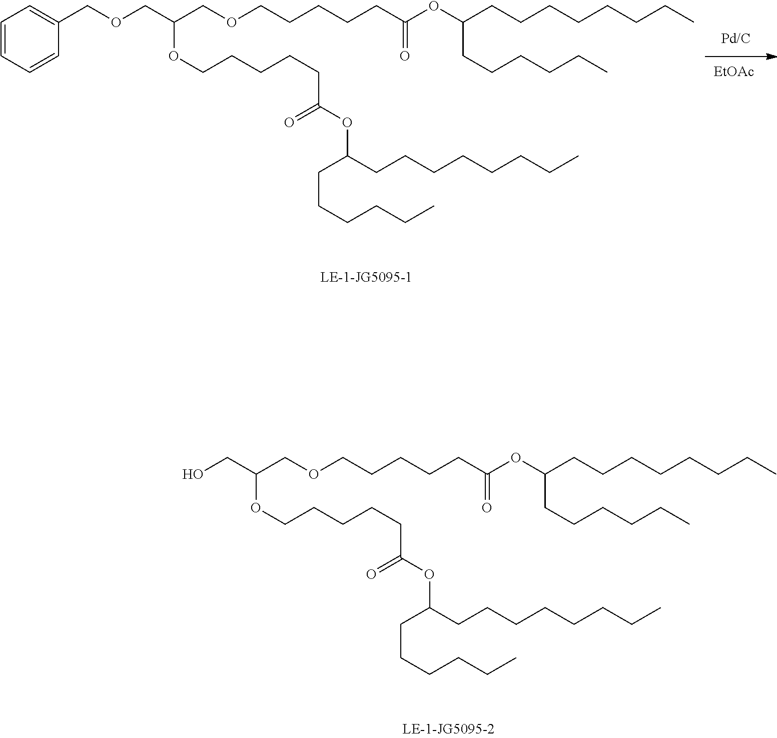 [SMILES: "EtOAc" ; CCCCCCCCC(CCCCCC)OC(=O)CCCCCOCC(COCc1ccccc1)OCCCCCC(=O)OC(CCCCCC)CCCCCCCC ; CCCCCCCCC(CCCCCC)OC(=O)CCCCCOCC(CO)OCCCCCC(=O)OC(CCCCCC)CCCCCCCC]

A solution of 1-hexylnonyl 6-[3-benzyloxy-2-[6-(1-hexylnonoxy)-6-oxo-hexoxy] propoxy] hexanoate (2.2 g, 2.12 mmol, purity: 80%) and Pd/C (0.5 g) were mixed with EtOAc (50 mL) and attached to a hydrogenation apparatus. The system was evacuated and then refilled with hydrogen. The reaction mixture was stirred for 16 h at room temperature. The mixture was filtered, and the filtrate was concentrated. The residue was purified by flash column chromatography on silica gel eluting with 1:2 ethyl acetate/petroleum ether to give 1-hexylnonyl 6-[2-[6-(1-hexylnonoxy)-6-oxo-hexoxy]-3-hydroxy-propoxy] hexanoate (1.4 g, 89%) as colorless oil.

1H NMR (400 MHZ, CDCl3) δ 4.86 (p, J=6.2 Hz, 2H), 3.77-3.69 (m, 1H), 3.66-3.39 (m, 8H), 2.29 (td, J=7.5, 2.2 Hz, 4H), 1.71-1.18 (m, 64H), 0.88 (t, J=6.8 Hz, 12H).

### Synthesis of the intermediate LE-1-JG5095-3

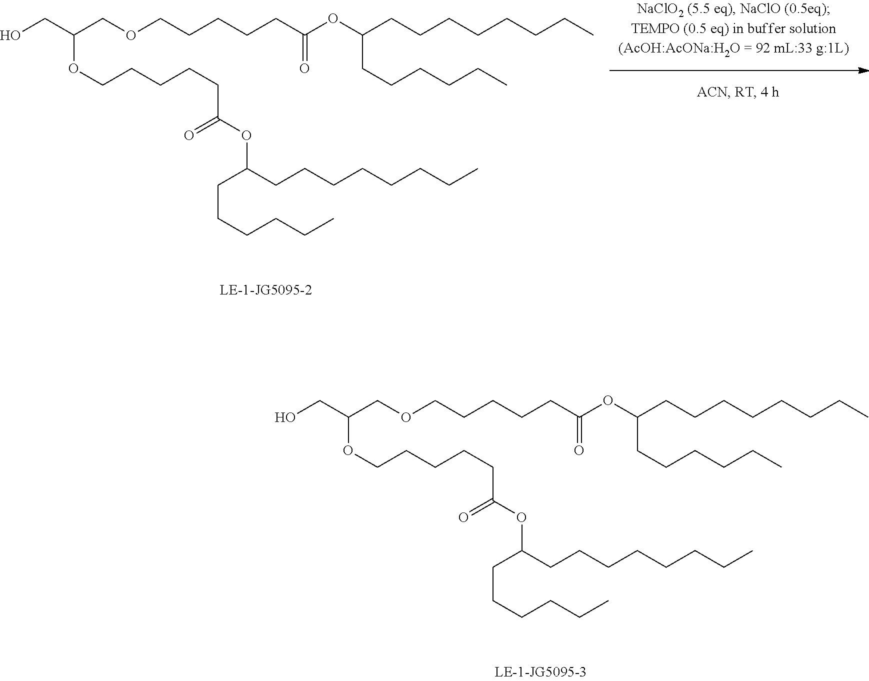 [SMILES: CCCCCCCCC(CCCCCC)OC(=O)CCCCCOCC(CO)OCCCCCC(=O)OC(CCCCCC)CCCCCCCC ; CCCCCCCCC(CCCCCC)OC(=O)CCCCCOCC(CO)OCCCCCC(=O)OC(CCCCCC)CCCCCCCC]

To a solution of 1-hexylnonyl 6-[2-[6-(1-hexylnonoxy)-6-oxo-hexoxy]-3-hydroxy-propoxy] hexanoate (1.4 g, 1.89 mmol) in Acetonitrile (AcCN, 50 mL) and PH=4-buffer solution (10 mL, AcOH: AcONa: water=92 mL: 33 g: 1000 mL) were added sodium chlorite (0.854 g, 9.44 mmol) and sodium hypochlorite (0.141 g, 0.95 mmol) and followed by TEMPO ((2,2,6,6-tetramethylpiperidin-1-yl)oxyl) (0.148 g, 0.95 mmol). The reaction became black and stirred at 20° C. for 4 hr. LCMS indicated a clean reaction. The reaction was quenched with 20 drops of methanol and was poured into water (40 mL), 1 N HCl (10 mL) and extracted with ethyl acetate. The organic layers were combined, washed with brine (60 mL), dried over anhydrous sodium sulfate, filtered and concentrated. The residue was purified through flash chromatography eluted with 5% to 10% methanol in dichloromethane to give 2,3-bis [6-(1-hexylnonoxy)-6-oxo-hexoxy] propanoic acid (1.2 g, 84.1% yield) as colorless oil.

1H NMR (400 MHZ, CDCl3) δ 4.94-4.79 (m, 2H), 4.09-3.99 (m, 1H), 3.84-3.40 (m, 6H), 2.29 (td, J=7.5, 3.7 Hz, 4H), 1.72-1.14 (m, 61H), 0.88 (t, J=6.7 Hz, 12H).

### Synthesis of the Intermediate LE-1-JG5095-4

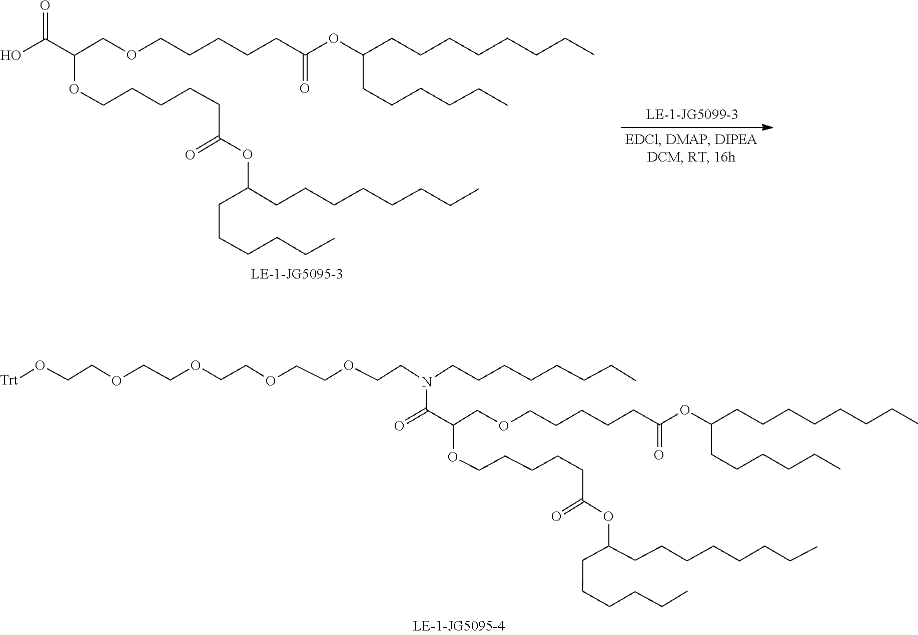

To a mixture of 2,3-bis [6-(1-hexylnonoxy)-6-oxo-hexoxy] propanoic acid (1.2 g, 1.59 mmol), EDC HCl (0.609 g, 3.18 mmol), DIEA (0.411 g, 3.18 mmol) in DCM (20 mL) were added N-[2-[2-[2-[2-(2-trityloxyethoxy) ethoxy]ethoxy] ethoxy]ethyl] octan-1-amine (1.03 g, 1.75 mmol) and N,N-dimethylpyridin-4-amine (0.039 g, 0.32 mmol). The mixture was stirred for 16 h at RT. The mixture was poured into DCM (200 mL) and washed with NaHCO₃ solution and brine, dried over Na2SO4 and concentrated. The residue was purified by flash column chromatography on silica gel eluting with 1:3 ethyl acetate/petroleum ether to give 1-hexylnonyl 6-[2-[6-(1-hexylnonoxy)-6-oxo-hexoxy]-3-[octyl-[2-[2-[2-[2-(2-trityloxyethoxy) ethoxy]ethoxy] ethoxy]ethyl]amino]-3-oxo-propoxy] hexanoate (1.2 g, 56.8%) as colorless oil.

¹H NMR (400 MHZ, CDCl3) δ 7.50-7.43 (m, 6H), 7.29 (dd, J=10.1, 4.8 Hz, 6H), 7.22 (dd, J=8.5, 6.0 Hz, 3H), 4.92-4.80 (m, 2H), 4.44-4.27 (m, 1H), 3.77-3.20 (m, 28H), 2.27 (t, J=7.6 Hz, 4H), 1.63-1.43 (m, 17H), 1.42-1.16 (m, 57H), 0.88 (dd, J=8.4, 5.1 Hz, 15H).

### Synthesis of the Intermediate LE-1-JG5095-5

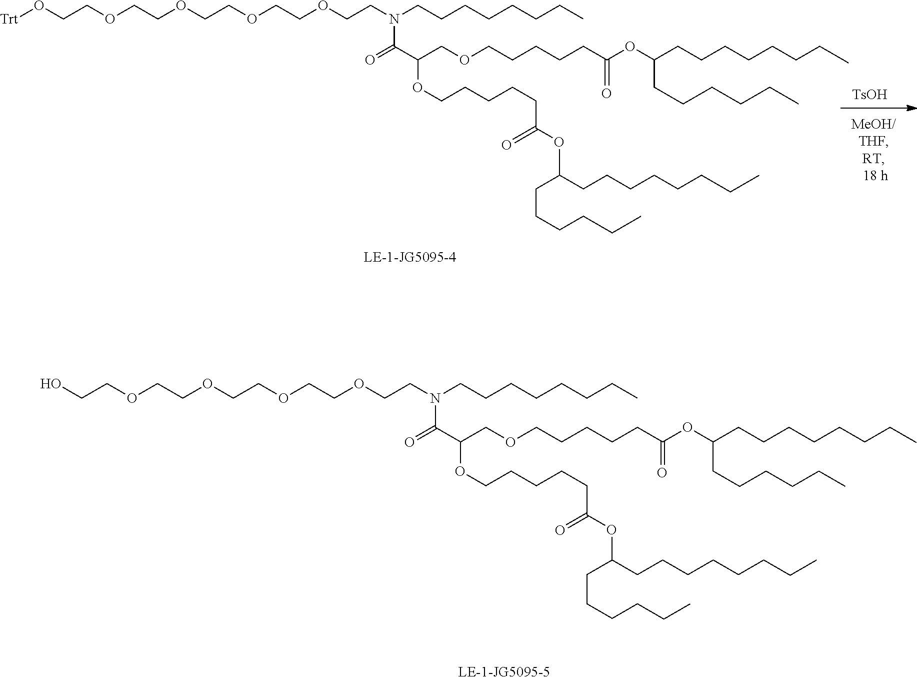

To a solution of 1-hexylnonyl 6-[2-[6-(1-hexylnonoxy)-6-oxo-hexoxy]-3-[octyl-[2-[2-[2-[2-(2-trityloxyethoxy) ethoxy]ethoxy] ethoxy]ethyl]amino]-3-oxo-propoxy] hexanoate (1.2 g, 0.903 mmol) in methanol/THF (20 mL, 1/1 v/v) was added 4-methylbenzenesulfonic acid (0.466 g, 2.71 mmol) in one portion at room temperature and the mixture was stirred at room temperature for 18 h. TLC (4% ethyl acetate in petroleum ether) indicated that the starting material was disappeared completely. 2 mL triethylamine was added to quench the reaction and the solvent was removed under vacuum. The residue was purified by flash chromatography eluted with 0% to 5% MeOH in DCM (4%) to give 1-hexylnonyl6-[2-[6-(1-hexylnonoxy)-6-oxo-hexoxy]-3-[2-[2-[2-[2-(2-hydroxyethoxy) ethoxy]ethoxy] ethoxy]ethyl-octyl-amino]-3-oxo-propoxy] hexanoate (0.7 g, 71.3% yield) as colorless oil.

1H NMR (400 MHZ, CDCl3) δ 4.90-4.80 (m, 2H), 4.45-4.27 (m, 1H), 3.91-3.18 (m, 29H), 2.27 (t, J=7.6 Hz, 4H), 1.68-1.44 (m, 19H), 1.41-1.17 (m, 56H), 0.95-0.81 (m, 15H).

### Synthesis of the Compound XXX

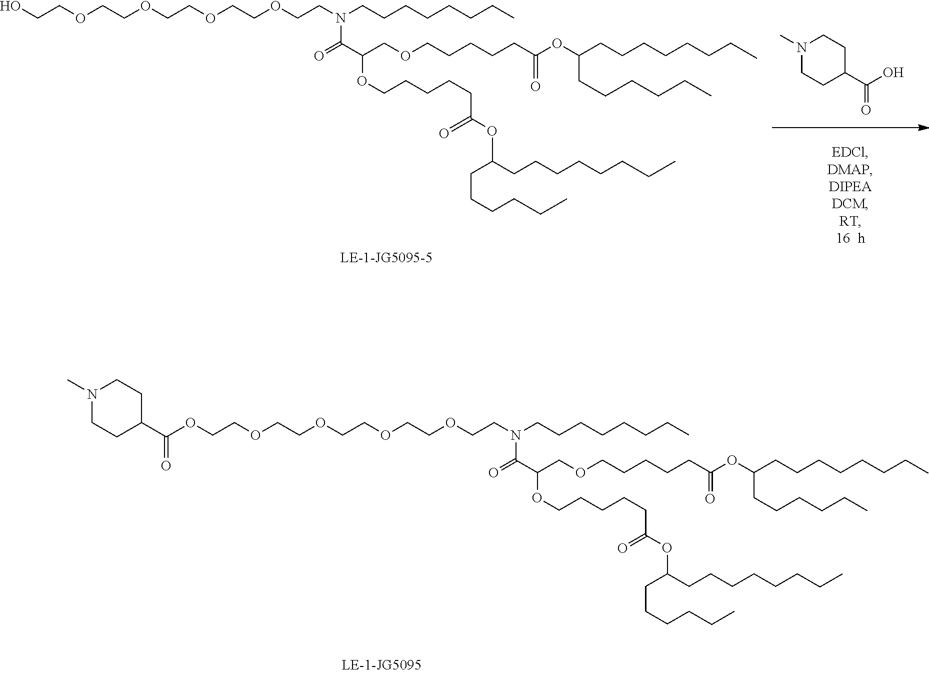

To the solution of 1-hexylnonyl 6-[2-[6-(1-hexylnonoxy)-6-oxo-hexoxy]-3-[2-[2-[2-[2-(2-hydroxyethoxy) ethoxy]ethoxy] ethoxy]ethyl-octyl-amino]-3-oxo-propoxy] hexanoate (0.7 g, 0.644 mmol) and 1-methylpiperidine-4-carboxylic acid (0.277 g, 1.93 mmol) in dry dichloromethane (20 mL) were added DIPEA (0.25 g, 1.93 mmol), DMAP (0.016 g, 0.129 mmol) and followed by under ice bath EDCl (0.37 g, 1.93 mmol) portion wise. The mixture was stirred at room temperature for 16 h. The reaction was diluted with dichloromethane and washed with brine. The organic layer was dried over sodium sulfate, filtered and concentrated. The residue was purified by flash chromatography eluted with 0% to 10% methanol in dichloromethane to give 2-[2-[2-[2-[2-[2,3-bis [6-(1-hexylnonoxy)-6-oxo-hexoxy] propanoyl-octyl-amino]ethoxy]ethoxy] ethoxy]ethoxy]ethyl1-methylpiperidine-4-carboxylate (0.318 g, 64.6% yield) as colorless oil.

1H NMR (400 MHZ, CDCl3) δ 4.90-4.81 (m, 2H), 4.43-4.28 (m, 1H), 4.26-4.21 (m, 2H), 3.75-3.24 (m, 27H), 2.83 (d, J=13.4 Hz, 2H), 2.42-2.23 (m, 8H), 2.10-1.73 (m, 10H), 1.70-1.18 (m, 77H), 0.94-0.83 (m, 15H).

### Example 23: synthesis of 2-[2-[2-[2-[2-[2,3-bis [6-(1-octylnonoxy)-6-oxo-hexoxy] propanoyl-octyl-amino]ethoxy]ethoxy] ethoxy]ethoxy]ethyl 1-methylpiperidine-4-carboxylate (compound XXXI)

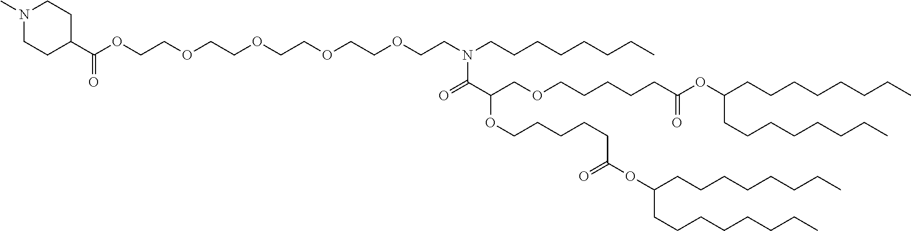 [SMILES: CCCCCCCCC(CCCCCCCC)OC(=O)CCCCCOCC(OCCCCCC(=O)OC(CCCCCCCC)CCCCCCCC)C(=O)N(CCCCCCCC)CCOCCOCCOCCOCCOC(=O)C1CCN(C)CC1]

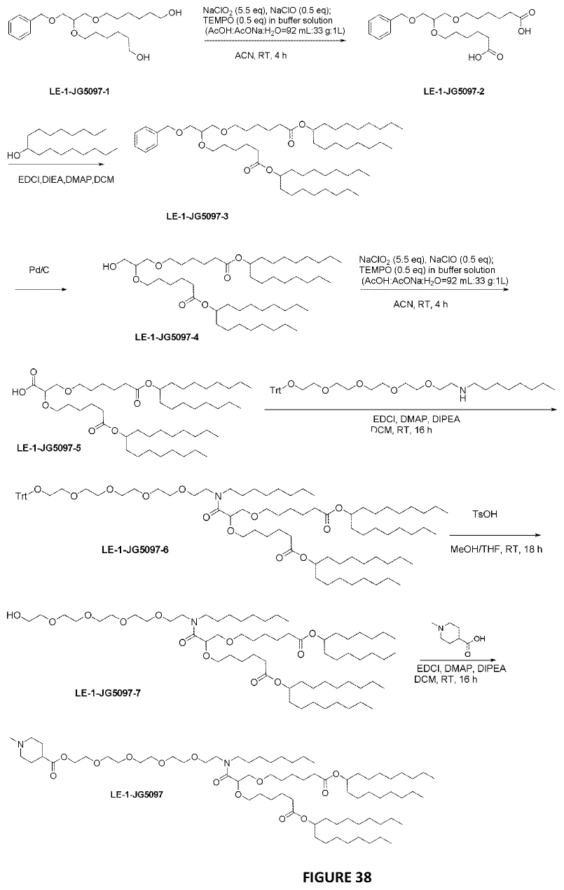

The compound XXXI is prepared according to the schema of synthesis of FIG. 38.

### Synthesis of the Intermediate LE-1-JG5097-2

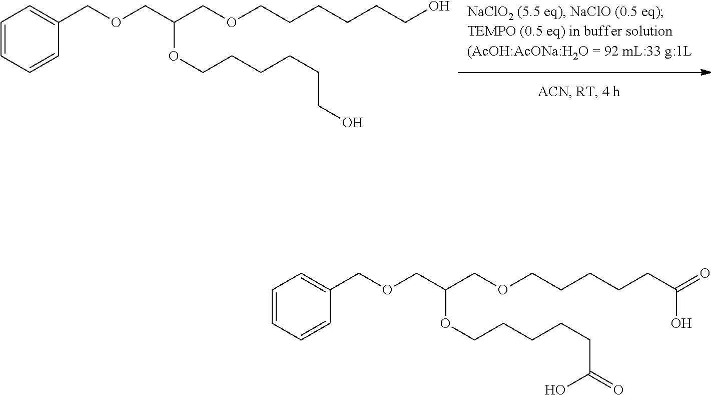 [SMILES: OCCCCCCOCC(COCc1ccccc1)OCCCCCCO ; O=C(O)CCCCCOCC(COCc1ccccc1)OCCCCCC(=O)O]

A solution of 6-[3-benzyloxy-2-(6-hydroxyhexoxy) propoxy] hexan-1-ol (7 g, 18.3 mmol) dissolved in ACN (80 ml) and added 5 ml buffer solution adjust PH to 4. Then added sodium chlorite (18.2 g, 201 mmol) and chlorosylsodium (1.36 g, 18.3 mmol), TEMPO (2.86 g, 18.3 mmol) was stirred at RT for 4 h. TLC (PE:EA=1:1, RF: 0.3) indicated raw materials was consumed. The mixture quenched with 5 ml methanol and was poured into water (80 ml) and extracted with ethyl acetate (80 ml×3). The organic layers were combined, washed with brine (80 ml), dried over anhydrous sodium sulfate, filtered and concentrated. The residue, combined with above batch, was purified through flash chromatography eluted with 5% to 10% methanol in dichloromethane to give 6-[3-benzyloxy-2-(5-carboxypentoxy) propoxy] hexanoic acid (6.2 g, 82.5% yield) as light yellow oil.

1H NMR (400 MHZ, CDCl3) δ 7.37-7.27 (m, 5H), 4.54 (s, 2H), 3.62-3.41 (m, 9H), 2.35 (t, J=7.1 Hz, 4H), 1.71-1.52 (m, 8H), 1.46-1.34 (m, 4H).

### Synthesis of the Intermediate LE-1-JG5097-3

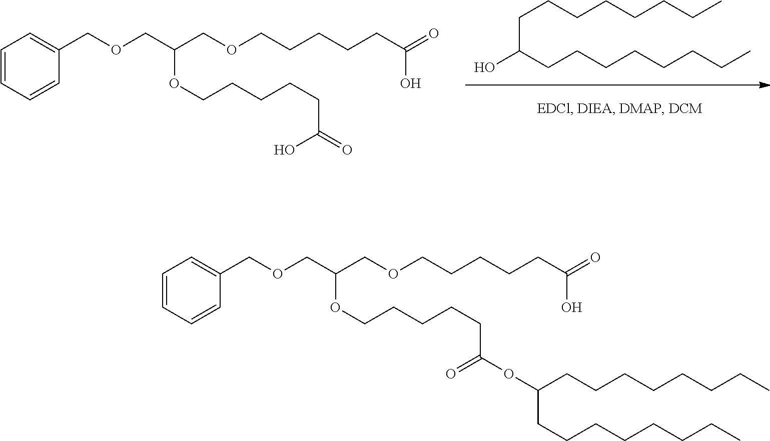 [SMILES: O=C(O)CCCCCOCC(COCc1ccccc1)OCCCCCC(=O)O ; CCCCCCCCC(O)CCCCCCCC ; CCCCCCCCC(CCCCCCCC)OC(=O)CCCCCOC(COCCCCCC(=O)O)COCc1ccccc1]

A mixture of 6-[3-benzyloxy-2-(5-carboxypentoxy) propoxy] hexanoic acid (1.0 g, 2.4 mmol), heptadecan-9-ol (2.5 g, 9.74 mmol), N,N-dimethylpyridin-4-amine (0.06 g, 0.4 mmol), heptadecan-9-ol (1.26 g, 9.7 mmol), 3-(ethyliminomethyleneamino)-N,N-dimethyl-propan-1-amine;hydrochloride (1.4 g, 7.31 mmol) in DCM (100 mL) was stirred at room temperature 16 hrs. TLC (PE:EA=10:1, RF: 0.9) indicated raw materials was consumed. The reaction was poured in 1 M HCl (30 ml) and extracted with ethyl acetate (60 ml×3). The aqueous layer was exacted with ethyl acetate again. The combined organic layers were dried over sodium sulfate, filtered, and concentrated. Then purified by column chromatography with ethyl acetate in petroleum ether (5-10%) to give to 1-octylnonyl 6-[3-benzyloxy-2-[6-(1-octylnonoxy)-6-oxo-hexoxy] propoxy] hexanoate (2.0 g, crude) as an light solid.

### Synthesis of Intermediate LE-1-JG5097-4

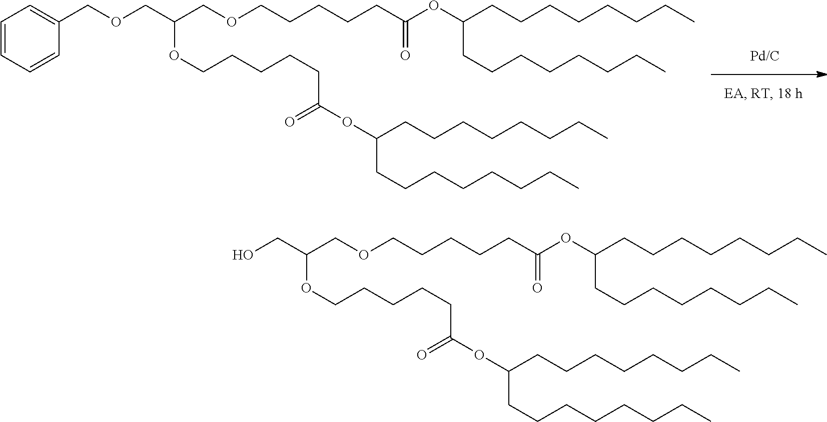 [SMILES: CCCCCCCCC(CCCCCCCC)OC(=O)CCCCCOCC(COCc1ccccc1)OCCCCCC(=O)OC(CCCCCCCC)CCCCCCCC ; CCCCCCCCC(CCCCCCCC)OC(=O)CCCCCOCC(CO)OCCCCCC(=O)OC(CCCCCCCC)CCCCCCCC]

A solution of 1-octylnonyl 6-[3-benzyloxy-2-[6-(1-octylnonoxy)-6-oxo-hexoxy] propoxy] hexanoate (2.0 g, 2.25 mmol) in ethyl acetate (20 mL) was added Pd-C (0.12 g, 1.13 mmol, 10% on activated wood carbon, 50% water wet) in a 100 ml glass bottle and stirred for overnight at room temperature under H₂ balloon. TLC (PE:EA=10:1, Rf: ˜ 0.5, Phosphomolybdic acid) showed the raw material disappeared. Filtered and the filtrate was concentrated to give 1-octylnonyl 6-[3-hydroxy-2-[6-(1-octylnonoxy)-6-oxo-hexoxy] propoxy] hexanoate (1.1 g, purity: 95%, yield: 61.2%) as an oil.

### Synthesis of Intermediate LE-1-JG5097-5

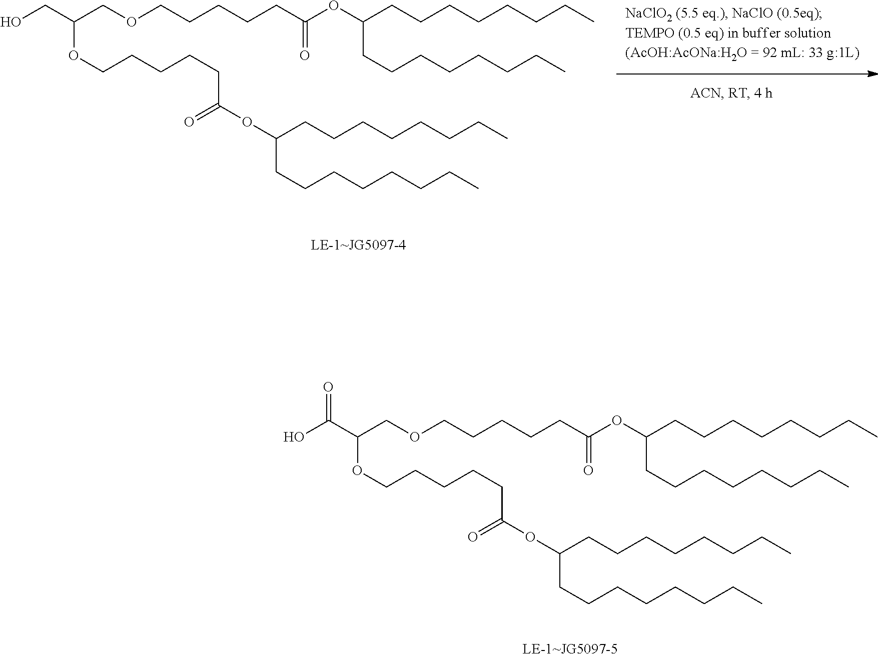 [SMILES: CCCCCCCCC(CCCCCCCC)OC(=O)CCCCCOCC(CO)OCCCCCC(=O)OC(CCCCCCCC)CCCCCCCC ; CCCCCCCCC(CCCCCCCC)OC(=O)CCCCCOCC(OCCCCCC(=O)OC(CCCCCCCC)CCCCCCCC)C(=O)O]

A solution of 1-octylnonyl 6-[3-hydroxy-2-[6-(1-octylnonoxy)-6-oxo-hexoxy] propoxy] hexanoate (1.15 g, 1.44 mmol) dissolved in ACN (10 ml) and added 5 ml buffer solution adjust pH to 4. Then added sodium chlorite (7.93 g, 0.72 mmol) and Sodium hypochlorite (0.05 g, 0.7 mmol), TEMPO (0.113 g, 0.72 mmol) was stirred at RT for 4 h. TLC (PE: ACE=2:1, RF: 0.3) indicated raw materials was consumed. The mixture quenched with 5 ml methanol and was poured into water (20 ml) and extracted with ethyl acetate (20 ml×3). The organic layers were combined, washed with brine (20 ml), dried over anhydrous sodium sulfate, filtered and concentrated. The residue, combined with above batch, was purified through flash chromatography eluted with 10% to 20% acetone in petroleum ether to give 2,3-bis [6-(1-octylnonoxy)-6-oxo-hexoxy] propanoic acid (0.57 g, 48.7% yield) as light yellow oil.

### Synthesis of Intermediate LE-1-JG5097-6

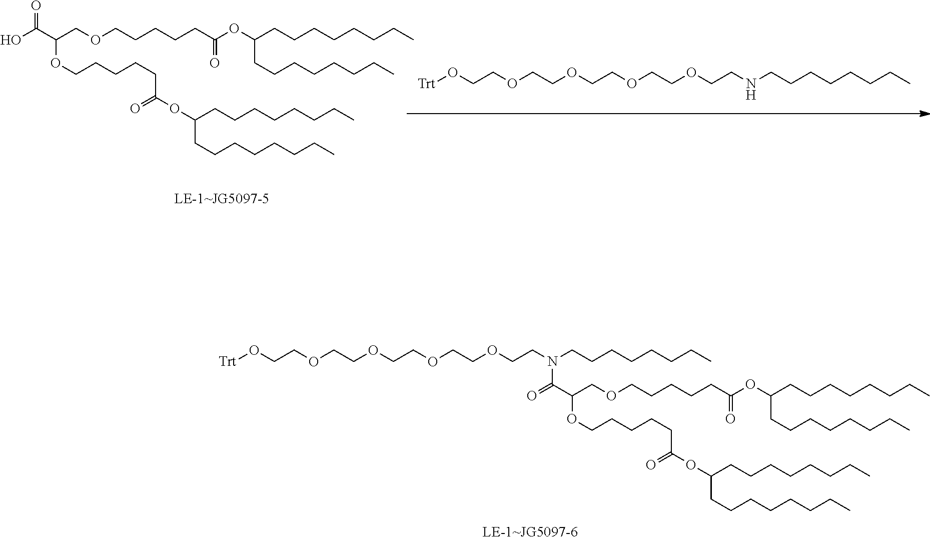

A solution of 2,3-bis [6-(1-octylnonoxy)-6-oxo-hexoxy] propanoic acid (570 mg, 0.703 mmol), N-[2-[2-[2-[2-(2-trityloxyethoxy) ethoxy]ethoxy] ethoxy]ethyl] octan-1-amine (541 mg, 0.913 mmol),N,N-dimethylpyridin-4-amine (0.62 g, 5 mmol), N-ethyl-N-isopropyl-propan-2-amine (6.6 g, 51.2 mmol), 3-(ethyliminomethyleneamino)-N,N-dimethyl-propan-1-amine;hydrochloride (269 mg, 1.41 mmol) was dissolved at DCM (100 ml) and stirred at RT for 16 h. TLC (PE:EA=2:1, RF: 0.8, iodine) indicated raw materials was consumed. The reaction was poured into dilute HCl (50 mL, 1 mol/L) and extracted with dichloromethane (60 ml×3). The organic layers were combined, washed with brine (60 mL), dried over anhydrous sodium sulfate, filtered, and concentrated. The residue, combined with above batch, was purified through flash chromatography eluted with 5% to 10% acetone in petroleum ether to give 1-octylnonyl 6-[2-[6-(1-octylnonoxy)-6-oxo-hexoxy]-3-[octyl-[2-[2-[2-[2-(2-trityloxyethoxy) ethoxy]ethoxy] ethoxy]ethyl]amino]-3-oxo-propoxy] hexanoate (437 mg, 44.9% yield) as a pure oil.

### Synthesis of Intermediate LE-1-JG5097-7

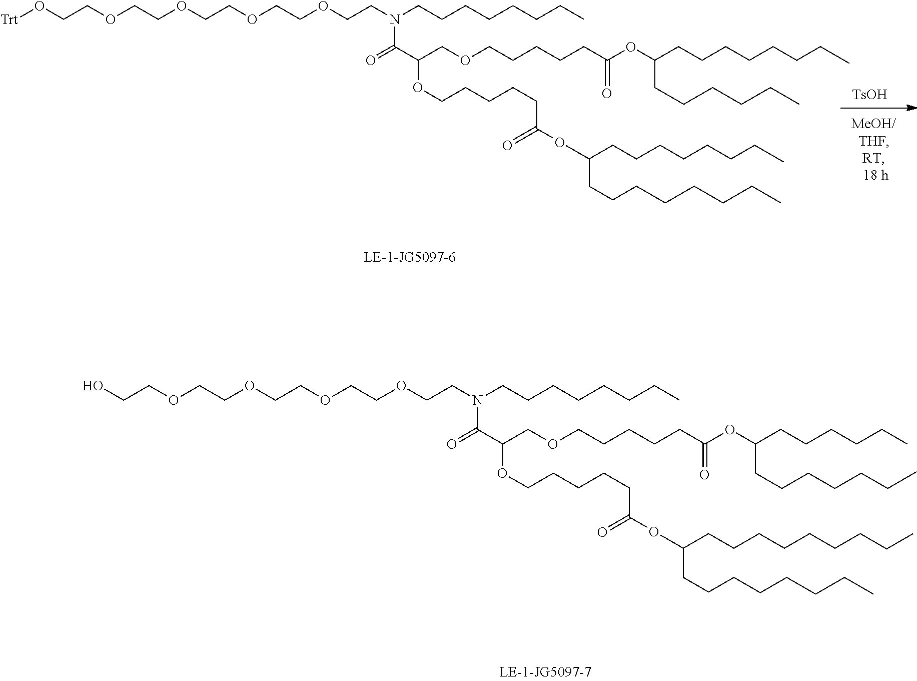

A solution of 1-octylnonyl 6-[2-[6-(1-octylnonoxy)-6-oxo-hexoxy]-3-[octyl-[2-[2-[2-[2-(2-trityloxyethoxy) ethoxy]ethoxy] ethoxy]ethyl]amino]-3-oxo-propoxy] hexanoate (353 mg, 0.255 mmol) and 4-methylbenzenesulfonic acid (0.117 g, 0.6 mmol) was stirred at methanol/THF (1:1, 10 ml) for 16 h. TLC (PE: EA). The reaction was added triethylamine 2 ml neutralize 4-methylbenzenesulfonic acid and was poured into dilute HCl (50 mL, 1 mol/L) and extracted with dichloromethane (60 ml×3). The organic layers were combined, washed with brine (60 mL), dried over anhydrous sodium sulfate, filtered, and concentrated. The residue, combined with above batch, was purified through flash chromatography eluted with 13% PE in ACE to give 1-octylnonyl 6-[3-[2-[2-[2-[2-(2-hydroxyethoxy) ethoxy]ethoxy] ethoxy]ethyl-octyl-amino]-2-[6-(1-octylnonoxy)-6-oxo-hexoxy]-3-oxo-propoxy] hexanoate (0.27 g, 92.7% yield) as a yellow oil.

### Synthesis of Compound XXXI

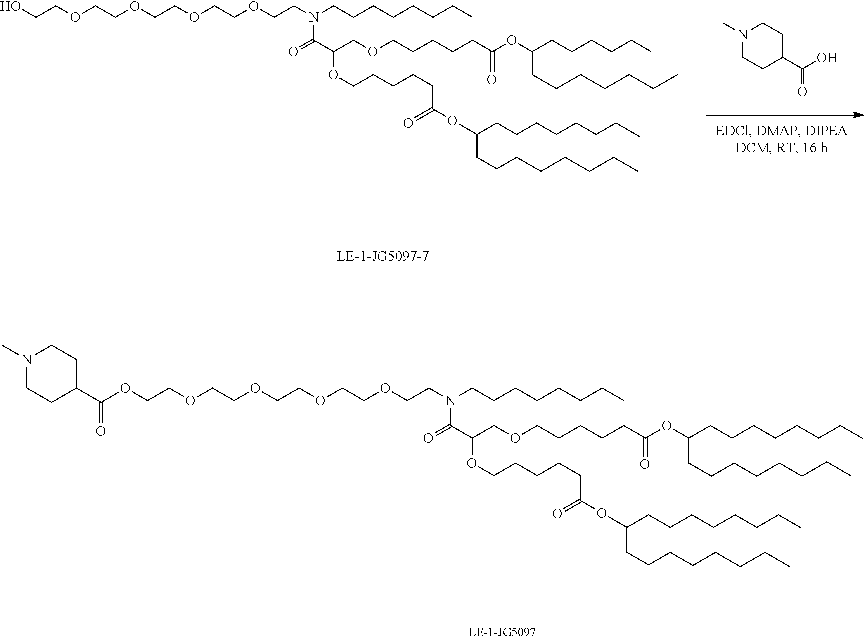

A mixture of 1-octylnonyl 6-[3-[2-[2-[2-[2-(2-hydroxyethoxy) ethoxy]ethoxy] ethoxy]ethyl-octyl-amino]-2-[6-(1-octylnonoxy)-6-oxo-hexoxy]-3-oxo-propoxy] hexanoate (0.47 g, 0.4 mmol), N,N-dimethylpyridin-4-amine (0.01 g, 0.08 mmol), 1-methylpiperidine-4-carboxylic acid (0.17 g, 1.23 mmol), 3-(ethyliminomethyleneamino)-N,N-dimethyl-propan-1-amine;hydrochloride (0.23 g, 1.23 mmol),N-ethyl-N-isopropyl-propan-2-amine (0.15 g, 1.23 mmol) in DCM (10 mL) was stirred at room temperature 16 hrs. TLC (DCM:MeOH=10:1, RF: 0.4, iodine). The reaction was poured in 1 M HCl (30 ml) and extracted with ethyl acetate (60 ml×3). The aqueous layer was exacted with ethyl acetate again. The combined organic layers were dried over sodium sulfate, filtered, and concentrated. Then purified by column chromatography with DCM in MeOH (5-7%) to give to 2-[2-[2-[2-[2-[2,3-bis [6-(1-octylnonoxy)-6-oxo-hexoxy] propanoyl-octyl-amino]ethoxy]ethoxy] ethoxy]ethoxy]ethyl 1-methylpiperidine-4-carboxylate (0.47 g, 90.1% yield) as pale yellow oil.

### Example 24: synthesis of 2-[2-[2-[2-[(2S)-2,3-bis [8-(1-octylnonoxy)-8-oxo-octoxy] propoxy] ethoxy]ethoxy] ethoxy]ethyl 1-methylpiperidine-4-carboxylate (compound XXXII)

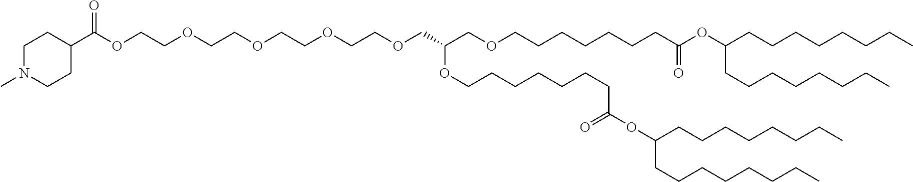 [SMILES: CCCCCCCCC(CCCCCCCC)OC(=O)CCCCCCCOC[C@@H](COCCOCCOCCOCCOC(=O)C1CCN(C)CC1)OCCCCCCCC(=O)OC(CCCCCCCC)CCCCCCCC]

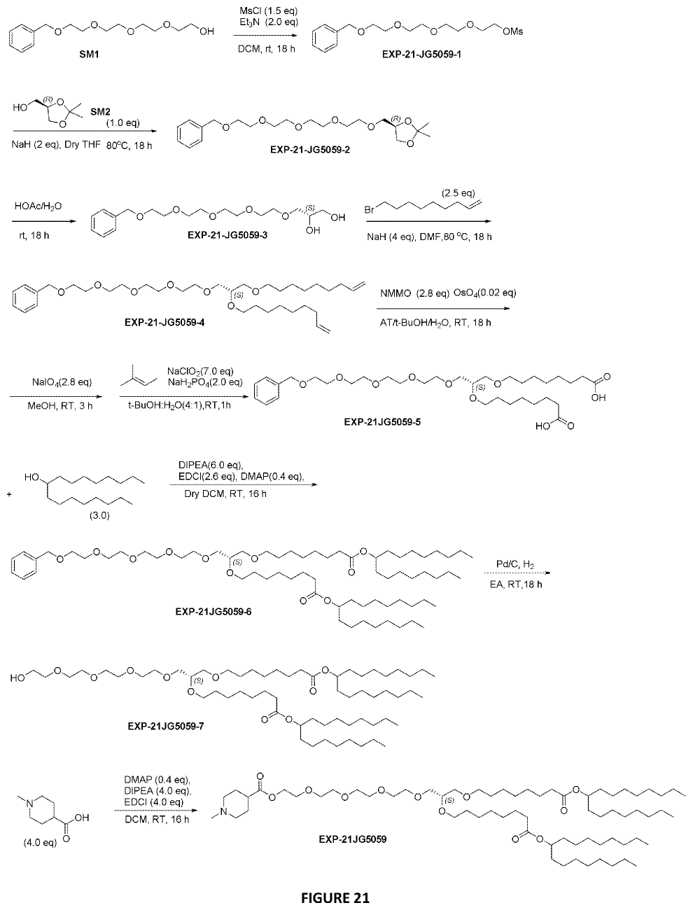

The compound XXXII is prepared according to the schema of synthesis of FIG. 21.

### Synthesis of the Intermediate LE-1-JG5059-1

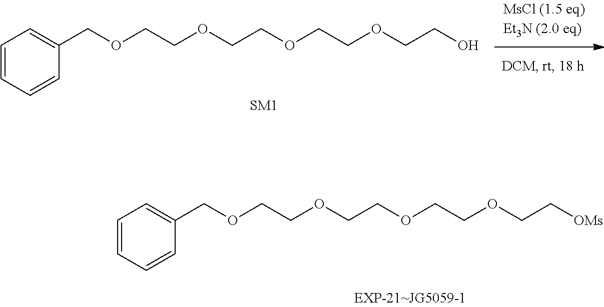 [SMILES: OCCOCCOCCOCCOCc1ccccc1 ; CS(=O)(=O)OCCOCCOCCOCCOCc1ccccc1]

To a mixture of 2-[2-[2-(2-benzyloxyethoxy) ethoxy]ethoxy] ethanol (50 g, 176 mmol) and N,N-diethylethanamine (17.8 g, 176 mmol) in DCM (500 mL) was added methanesulfonyl chloride (15.1 g, 132 mmol) slowly at 0° C. The mixture was then stirred overnight at room temperature. TLC (DCM:MeOH=5:1, Rf: ˜ 0.9, iodine) showed the raw material disappeared. The mixture was added DCM (400 ml) to the solution, and the mixture was washed with diluted HCl (1M, 500 mL). The mixture was shaken, the layers were separated, and the organic layer was collected. The organic layer was further washed with water (1000 mL) and brine (1000 mL) and dried over Na2SO4. Solvent was then removed to give 2-[2-[2-(2-benzyloxyethoxy) ethoxy]ethoxy]ethyl methanesulfonate (63 g, 321 mmol, 98.9% yield) as an orange oil.

¹H NMR (400 MHZ, CDCl3) δ 7.40-7.19 (m, 5H), 4.56 (s, 2H), 4.37-4.32 (m, 2H), 3.76-3.71 (m, 2H), 3.70-3.59 (m, 12H), 3.04 (d, J=10.0 Hz, 3H).

### Synthesis of the Intermediate LE-1-JG5059-2

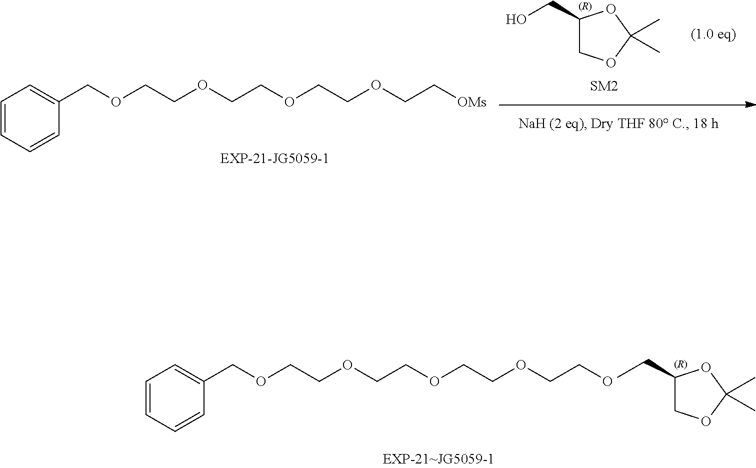

To the solution of [(4R)-2,2-dimethyl-1,3-dioxolan-4-yl]methanol (24.2 g, 174 mmol) in THF (1200 mL) was added NaH (23.1 g, 348 mmol) and the mixture was heated to 80° C. for 30 min. Then the reaction mixture was cooled to room temperature and 2-[2-[2-(2-benzyloxyethoxy) ethoxy]ethoxy]ethyl methanesulfonate (63 g, 174 mmol) was added under nitrogen and the reaction was heated at 80° C. for 24 h. TLC (DCM MeOH=10:1, Rf: ˜ 0.5, iodine) indicated that the starting material was consumed. The reaction was quenched with water (200 mL) and extracted with ethyl acetate (1000 mL). The aqueous layer was extracted with ethyl acetate (500 mL) again. The combined organic layers were washed with brine, dried over sodium sulfate, filtered and concentrated. The residue was purified through flash chromatography eluted with 50% petroleum ether in ethyl acetate to give (4R)-4-[2-[2-[2-(2-benzyloxyethoxy) ethoxy]ethoxy] ethoxymethyl]-2,2-dimethyl-1,3-dioxolane (48 g, 114 mmol, 65.8% yield) as light yellow oil.

1HNMR (400 MHZ, CDCl3) δ 7.39-7.25 (m, 5H), 4.57 (s, 2H), 3.76-3.35 (m, 25H), 2.33 (t, J=7.4 Hz, 4H), 1.69-1.48 (m, 8H), 1.34 (d, J=10.1 Hz, 12H).

### Synthesis of the Intermediate LE-1-JG5059-3

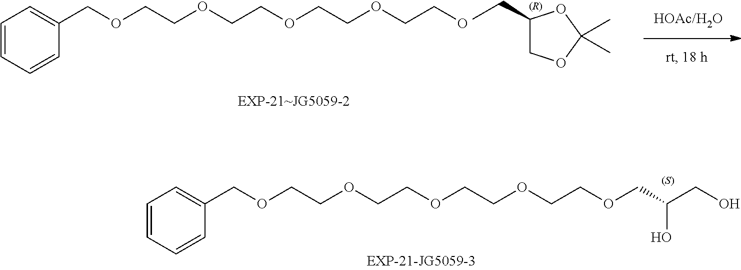

As a solution of (4R)-4-[2-[2-[2-(2-benzyloxyethoxy) ethoxy]ethoxy] ethoxymethyl]-2,2-dimethyl-1,3-dioxolane (48 g, 120 mmol) in HOAc (250 mL) and H₂O (250 mL) was stirred at room temperature for 18 h. TLC (DCM/MeOH 10/1, SM, Rf: 0.8; Product, Rf: 0.7) indicated that all the starting materials was consumed. The reaction was quenched with water (500 mL) and extracted with ethyl acetate (500 mL). The aqueous layer was extracted with ethyl acetate (500 mL) again. The combined organic layers were washed with brine, dried over sodium sulfate, filtered and concentrated. The residue was purified through flash chromatography eluted with 0 to 10% methanol in dichloromethane to give 3-[2-[2-[2-(2-benzyloxyethoxy) ethoxy]ethoxy] ethoxy]propane-1,2-diol (42 g, 117 mmol, 97.3% yield) as a yellow oil.

1H NMR (400 MHZ, CDCl3) δ 7.30 (dd, J=24.7, 4.8 Hz, 6H), 4.56 (s, 2H), 3.93-3.41 (m, 21H).

### Synthesis of the Intermediate LE-1-JG5059-4

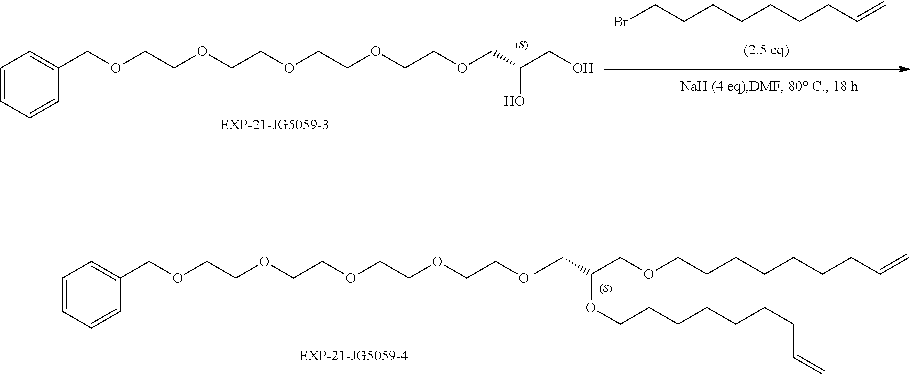

As a solution of (2S)-3-[2-[2-[2-(2-benzyloxyethoxy) ethoxy]ethoxy] ethoxy] propane-1,2-diol (42 g, 117 mmol) in dry DMF (400 mL) under nitrogen was added sodium hydride (18.6 g, 777 mmol) and the mixture was heated to 80° C. for 15 min. Then the reaction was cooled to room temperature and 9-bromonon-1-ene (60.1 g, 293 mmol) was added dropwise to this solution. The mixture was stirred at room temperature for 30 min and then was heated to 80° C. for 18h. TLC (50% Petroleum ether in Ethyl acetate, RF: 0.5, Phosphomolybdic acid) indicated that all starting materials was consumed. The reaction was quenched with water (70 mL) and partitioned between ethyl acetate and water. The aqueous layer was exacted with ethyl acetate again. The combined organic layers were dried over sodium sulfate, filtered and concentrated. The residue was purified by flash chromatography eluted with 20% ethyl acetate in petroleum ether to(S)-16-(non-8-en-1-yloxy)-1-phenyl-2,5,8,11,14,18-hexaoxaheptacos-26-ene (25.0 g, 41.2 mmol, 35.2% yield) as a yellow oil.

1H NMR (400 MHZ, CDCl3) δ 7.30 (dd, J=24.7, 4.8 Hz, 6H), 4.56 (s, 2H), 3.93-3.41 (m, 21H).

### Synthesis of the Intermediate LE-1-JG5059-5

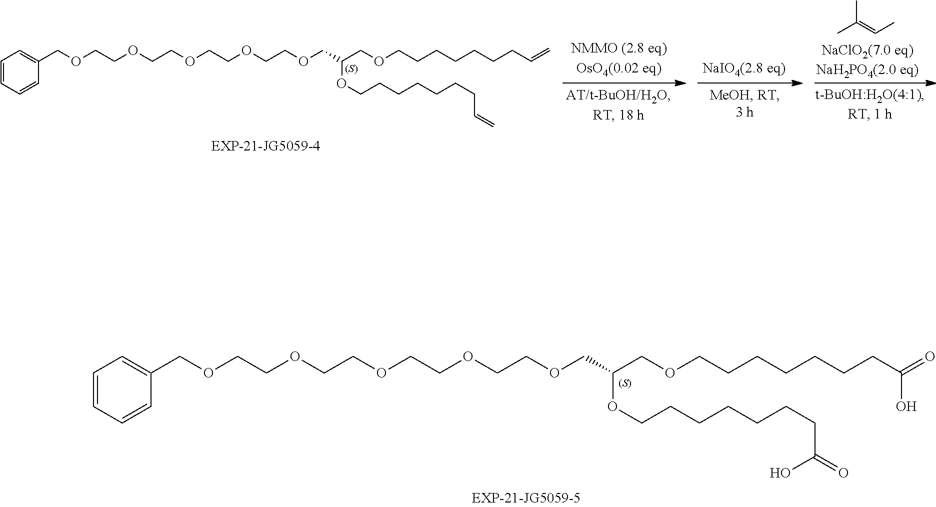

A solution of 2-[2-[2-[2-[(2S)-2,3-bis (non-8-enoxy) propoxy] ethoxy]ethoxy] ethoxy]ethoxybenzene (25 g, 41.8 mmol), 4-methyl-4-oxido-morpholin-4-ium (13.5 g, 115 mmol) and tetraoxoosmium (0.2 g, 0.84 mmol) in acetone (200 mL), t-BuOH (40 mL), and H₂O (20 mL) was stirred at room temperature for 12 h. TLC (DCM:MeOH=10:1, Rf: ˜ 0.5, Phosphomolybdic acid) indicated that all starting materials was consumed. After the mixture was concentrated, the residue was dissolved in EA and H₂O. The organic solution was separated, washed with brine, and dried Na2SO4. Evaporation of the solvent gave a crude glycol. A mixture of the crude glycol and NalO₄ (25.3 g, 118 mmol) in MeOH (250 mL) was stirred at room temperature for 3 h before concentration of the reaction mixture. EA (200 mL) and H₂O (200 mL) were added to the residue. The organic solution was separated, washed with brine, and dried Na2SO4. Concentrated gave a crude aldehyde. To a solution of the crude aldehyde, 2-methyl-2butene (180 mL), and NaH₂PO₄*2H₂O (9.4 g, 78.4 mmol), in t-BuOH (200 mL) and H₂O (50 mL) was added NaClO₂ (24.7 g, 274.4 mmol). The solution was stirred at room temperature for 1 h and then EA was added. The organic solution was separated, washed with brine, and dried Na2SO4. Concentrated followed by silica gel column chromatography MeOH in DCM (8%) to give 8-[(2S)-2-(7-carboxyheptoxy)-3-[2-[2-[2-(2-phenoxyethoxy) ethoxy]ethoxy] ethoxy] propoxy] octanoic acid (7.0 g, 36.7% yield) as colorless oil.

1H NMR (400 MHZ, CDCl3) δ 7.39-7.20 (m, 5H), 4.57 (s, 2H), 3.77-3.35 (m, 25H), 2.32 (t, J=7.4 Hz, 4H), 1.71-1.46 (m, 8H), 1.32 (s, 12H).

### Synthesis of the Intermediate LE-1-JG5059-6

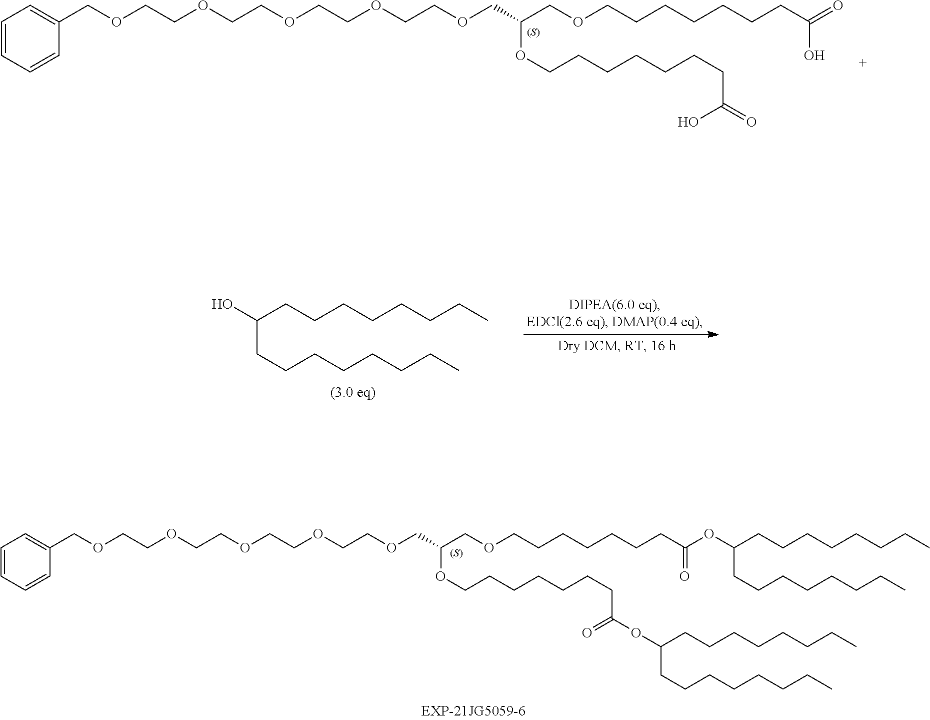

A mixture of 8-[(2S)-3-[2-[2-[2-(2-benzyloxyethoxy) ethoxy]ethoxy] ethoxy]-2-(7-carboxyheptoxy) propoxy] octanoic acid (5 g, 7.78 mmol), heptadecan-9-ol (7.98 g, 31.1 mmol), 3-(((ethylimino) methylene) amino)-N,N-dimethylpropan-1-amine hydrochloride (494 mg, 4.04 mmol),N,N-dimethylpyridin-4-amine (0.38 g, 3.11 mmol) and N-ethyl-N-isopropyl-propan-2-amine (6.03 g, 46.7 mmol) in DCM (50 mL) was stirred at room temperature 16 h. TLC (DCM:MeOH=10:1, Rf: ˜0.6, Phosphomolybdic acid).indicated that all starting materials was consumed. The reaction was poured into dilute HCl (50 mL, 1 mol/L) and extracted with dichloromethane (50 ml×3). The organic layers were combined, washed with brine (100 mL), dried over anhydrous sodium sulfate, filtered and concentrated. The residue, combined with above batch, was purified through flash chromatography eluted with 30% PE in EA to give 1-octylnonyl 8-[(2S)-3-[2-[2-[2-(2-benzyloxyethoxy) ethoxy]ethoxy] ethoxy]-2-[8-(1-octylnonoxy)-8-oxo-octoxy] propoxy] octanoate (1.0 g, 10.9% yield) as pale yellow oil.

1H NMR (400 MHZ, CDCl3) δ 7.36-7.27 (m, 4H), 4.90-4.79 (m, 2H), 4.57 (s, 2H), 3.75-3.35 (m, 25H), 2.27 (t, J=7.0 Hz, 4H), 1.66-1.42 (m, 18H), 1.28 (d, J=22.8 Hz, 60H), 0.88 (t, J=6.8 Hz, 12H).

### Synthesis of the Intermediate LE-1-JG5059-7

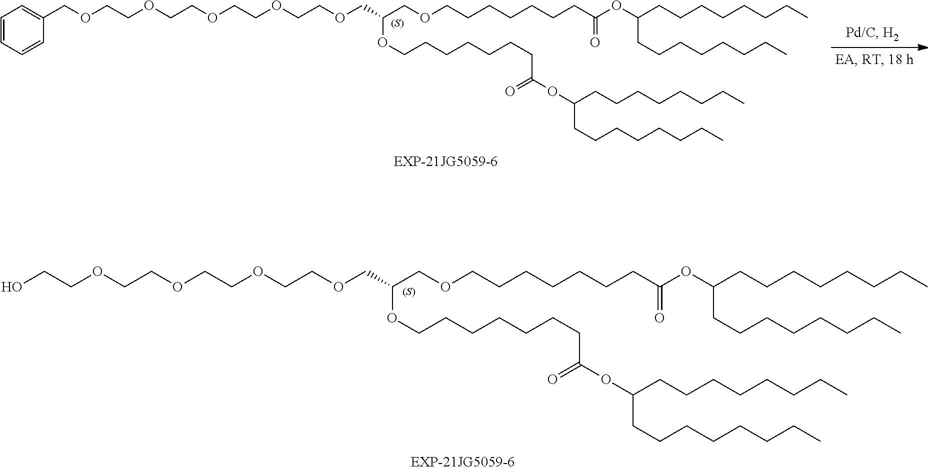

A solution of 1-octylnonyl 8-[(2S)-3-[2-[2-[2-(2-benzyloxyethoxy) ethoxy] ethoxy]ethoxy]-2-[8-(1-octylnonoxy)-8-oxo-octoxy] propoxy] octanoate (1.0 g, 0.893 mmol) was dissolved in 15 mL ethyl acetate. To the mixture was added Pd—C(400 mg, 10% on activated wood carbon, 50% water wet) in a 50 ml glass bottle and stirred for overnight at room temperature under H₂ balloon. TLC (DCM:MeOH=10:1), Rf: ˜ 0.5, Phosphomolybdic acid) showed the raw material disappeared. The mixture was then filtered and the filtrate was concentrated to give 1-octylnonyl 8-[(2S)-3-[2-[2-[2-(2-hydroxyethoxy) ethoxy]ethoxy] ethoxy]-2-[8-(1-octylnonoxy)-8-oxo-octoxy] propoxy] octanoate (0.8 g, 87.0% yield) as pale oil.

1H NMR (400 MHZ, CDCl3) δ 4.86 (p, J=6.2 Hz, 2H), 3.78-3.31 (m, 25H), 2.28 (dd, J=11.1, 3.9 Hz, 4H), 1.56 (ddd, J=22.7, 14.6, 6.1 Hz, 16H), 1.38-1.15 (m, 60H), 0.88 (t, J=6.8 Hz, 12H).

### Synthesis of the Compound XXXII

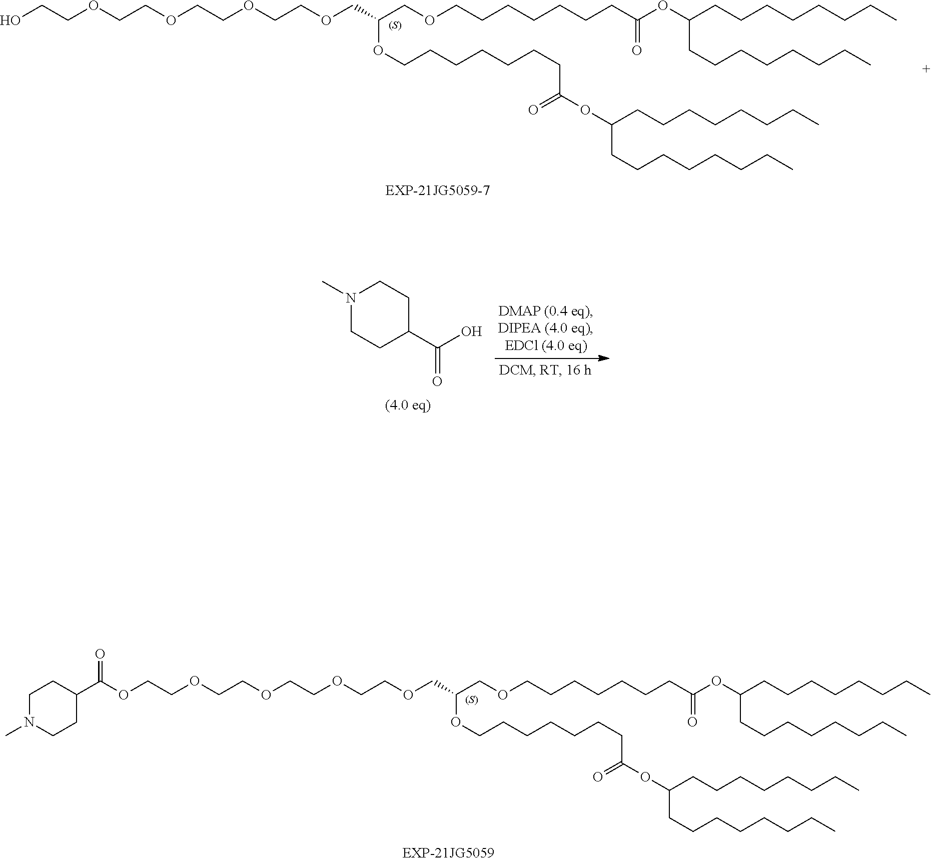

A mixture of 1-octylnonyl 8-[(2S)-3-[2-[2-[2-(2-hydroxyethoxy) ethoxy]ethoxy] ethoxy]-2-[8-(1-octylnonoxy)-8-oxo-octoxy] propoxy] octanoate (800 mg, 0.77 mmol), 3-(((ethylimino) methylene) amino)-N,N-dimethylpropan-1-amine hydrochloride (387 mg, 2.02 mmol), N,N-dimethylpyridin-4-amine (19 mg, 0.155 mmol) and N-ethyl-N-isopropyl-propan-2-amine (0.603 g, 4.66 mmol) in 20 mL DCM was stirred at room temperature 16 h. TLC (DCM:MeOH=10:1, Rf: ˜ 0.4, Phosphomolybdic acid) showed the raw material disappeared. The reaction was poured into brine (10 mL) and extracted with dichloromethane (10 ml×3). The organic layers were combined, dried over anhydrous sodium sulfate, filtered and concentrated. The residue, combined with above batch, Then purified by column chromatography with DCM in CH₃OH (0-10%) to give 2-[2-[2-[2-[(2S)-2,3-bis [8-(1-octylnonoxy)-8-oxo-octoxy] propoxy] ethoxy]ethoxy] ethoxy]ethyl-1-methylpiperidine-4-carboxylate (475 mg, 52.9%) as pale yellow oil.

1H NMR (400 MHZ, CDCl3) δ 4.92-4.78 (m, 2H), 4.28-4.18 (m, 2H), 3.70-3.42 (m, 23H), 2.85 (d, J=11.2 Hz, 2H), 2.37-2.23 (m, 8H), 1.82 (d, J=10.8 Hz, 2H), 1.64-1.46 (m, 18H), 1.38-1.21 (m, 63H), 0.88 (t, J=6.8 Hz, 12H).

### Example 25: Synthesis of 2-[2-[2-[2-[(2R)-2,3-bis [8-(1-octylnonoxy)-8-oxo-octoxy] propoxy] ethoxy]ethoxy] ethoxy]ethyl 1-methylpiperidine-4-carboxylate (compound XXXIII)

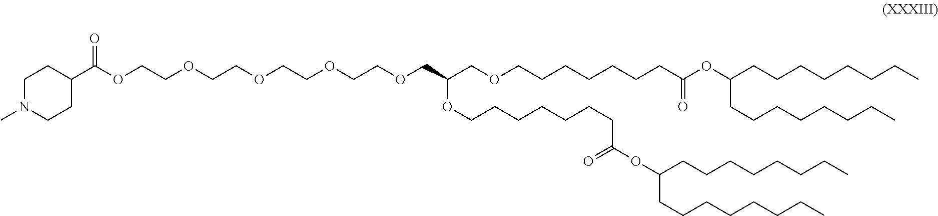 [SMILES: CCCCCCCCC(CCCCCCCC)OC(=O)CCCCCCCOC[C@H](COCCOCCOCCOCCOC(=O)C1CCN(C)CC1)OCCCCCCCC(=O)OC(CCCCCCCC)CCCCCCCC]

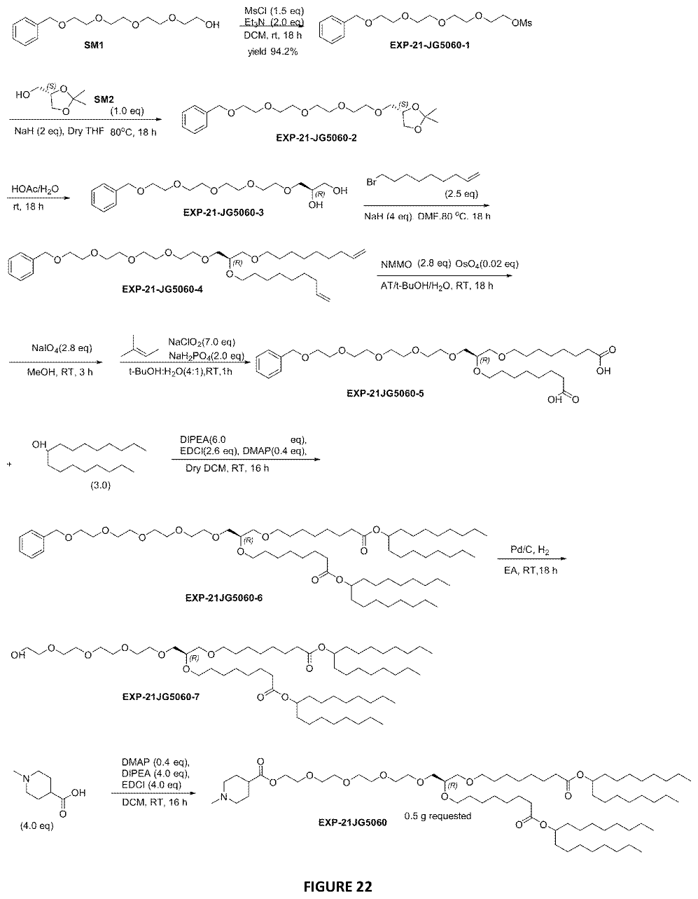

The compound XXXIII is prepared according to the schema of synthesis of FIG. 22.

### Synthesis of the Intermediate LE-1-JG5060-1

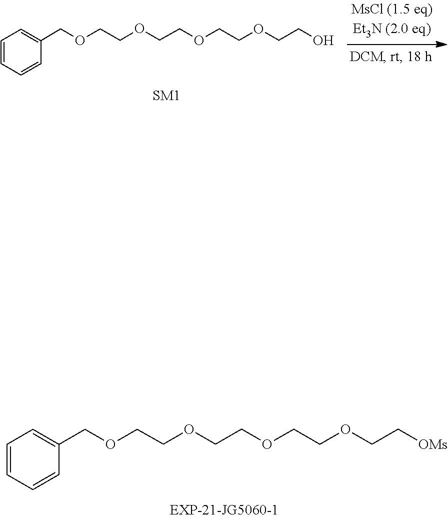 [SMILES: OCCOCCOCCOCCOCc1ccccc1 ; CS(=O)(=O)OCCOCCOCCOCCOCc1ccccc1]

To a mixture of 2-[2-[2-(2-benzyloxyethoxy) ethoxy]ethoxy] ethanol (50 g, 176 mmol) and N,N-diethylethanamine (17.8 g, 176 mmol) in DCM (500 mL) was added methanesulfonyl chloride (15.1 g, 132 mmol) slowly at 0° C. TLC (DCM:MeOH=5:1, Rf: ˜0.9,iodine) showed the raw material disappeared. The mixture was stirred overnight at room temperature. CH₂Cl₂ (400 mL) was added to the solution, and the mixture was washed with diluted HCl (1M, 500 mL). The mixture was shaken, the layers were separated, and the organic layer was collected. The organic layer was further washed with Water (1000 mL) and brine (1000 mL) and dried over Na2SO4. Solvent was then removed to give 2-[2-[2-(2-benzyloxyethoxy) ethoxy]ethoxy]ethyl methanesulfonate (63 g, 321 mmol, 98.9% yield) as a orange oil.

1H NMR (400 MHZ, CDCl3) δ 7.40-7.19 (m, 5H), 4.56 (s, 2H), 4.37-4.32 (m, 2H), 3.76-3.71 (m, 2H), 3.70-3.59 (m, 12H), 3.04 (d, J=10.0 Hz, 3H).

### Synthesis of the Intermediate LE-1-JG5060-2

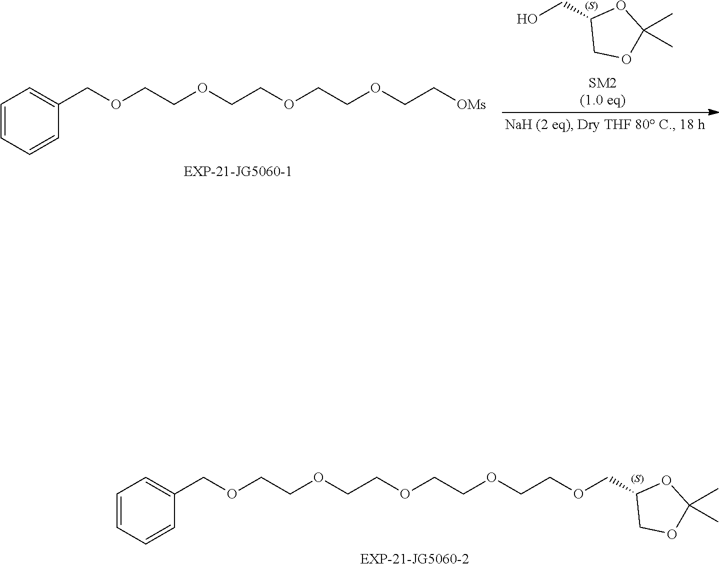

To the solution of(S)-(2,2-dimethyl-1,3-dioxolan-4-yl) methanol (23.6 g, 179 mmol) in THF (1200 mL) was added NaH (23.1 g, 348 mmol) and the mixture was heated to 80° C. for 30 min. Then the reaction was cooled to room temperature and 2-[2-[2-(2-benzyloxyethoxy) ethoxy]ethoxy]ethyl methanesulfonate (65 g, 179 mmol) was added under nitrogen and the reaction was heated at 80° C. for 18 h. TLC (DCM MeOH=10:1, Rf: ˜0.5, iodine) indicated that the starting material was consumed. The reaction was quenched with water (200 mL) and extracted with ethyl acetate (1000 mL). The aqueous layer was extracted with ethyl acetate (500 mL) again. The combined organic layers were washed with brine, dried over sodium sulfate, filtered and concentrated. The residue was purified through flash chromatography eluted with 50% petroleum ether in ethyl acetate to give(S)-2,2-dimethyl-4-(15-phenyl-2,5,8, 11,14-pentaoxapentadecyl)-1,3-dioxolane (54 g, 76.0% yield) as light-yellow oil.

1H NMR (400 MHZ, CDCl₃) δ 7.39-7.25 (m, 5H), 4.57 (s, 2H), 3.76-3.35 (m, 25H), 2.33 (t, J=7.4 Hz, 4H), 1.69-1.48 (m, 8H), 1.34 (d, J=10.1 Hz, 12H).

### Synthesis of the Intermediate LE-1-JG5060-3

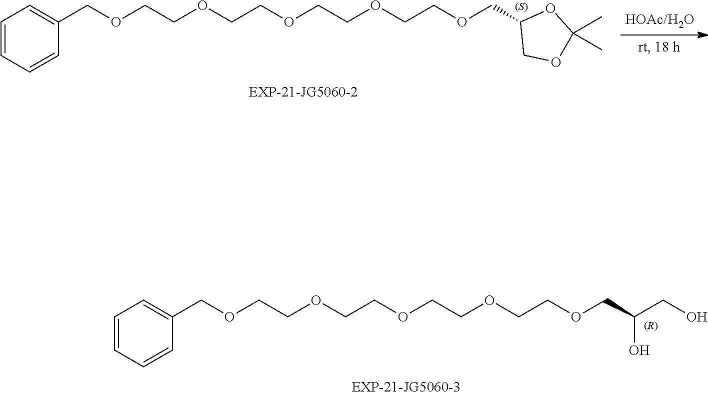

As a solution of(S)-2,2-dimethyl-4-(15-phenyl-2,5,8,11,14-pentaoxapentadecyl)-1,3-dioxolane (54 g, 136 mmol) in HOAc (250 ml) and H₂O (250 ml) was stirred at room temperature for 18 h.TLC (DCM/MeOH 10/1, SM, Rf: 0.8; Product: 0.7) indicated that all the starting materials was consumed. The solvent was removed under vacuum and extracted with toluene several times to give (R)-1-phenyl-2,5,8,11,14-pentaoxaheptadecane-16,17-diol (42 g, 87.5% yield) as a yellow oil.

1H NMR (400 MHZ, CDCl3) δ 7.30 (dd, J=24.7, 4.8 Hz, 6H), 4.56 (s, 2H), 3.93-3.41 (m, 21H).

### Synthesis of the Intermediate LE-1-JG5060-4

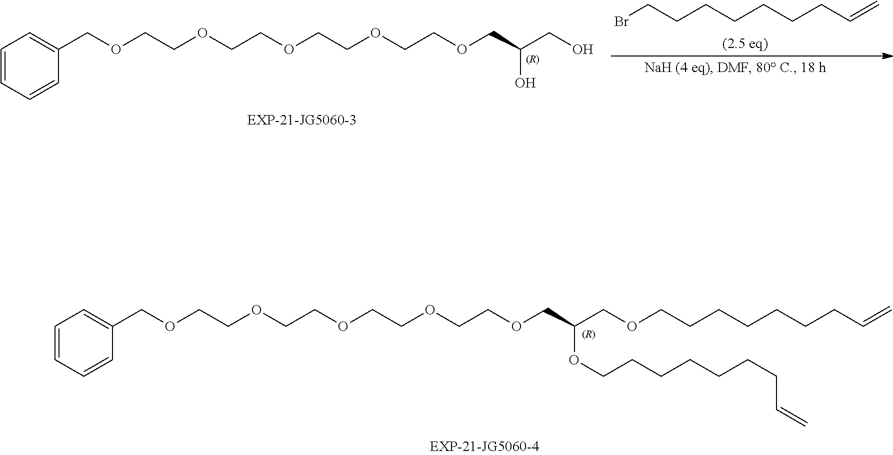

As a solution of (R)-1-phenyl-2,5,8,11,14-pentaoxaheptadecane-16,17-diol (42 g, 117 mmol) in dry DMF (400 ml) under nitrogen was added sodium; hydride (18.6 g, 777 mmol) and the mixture were heated to 80° C. for 15 min. Then the reaction cooled to room temperature and 9-bromonon-1-ene (60.1 g, 293 mmol) was added dropwise to this solution. The mixture was stirred at room temperature for 30 min and then was heated to 80° C. for 18 h. TLC (EA/PE=1/1, Rf: 0.8) indicated that a new spot was formed. The reaction was quenched with water (70 ml) and partitioned between ethyl acetate (400 mL) and water (400 mL). The aqueous layer was exacted with ethyl acetate (400 mL) again. The combined organic layers were dried over sodium sulfate, filtered and concentrated. The residue was purified by flash chromatography eluted with 20% ethyl acetate in petroleum ether to give (R)-16-(non-8-en-1-yloxy)-1-phenyl-2,5,8,11,14,18-hexaoxaheptacos-26-ene (25.0 g, 35.2% yield) as a yellow oil.

1H NMR (400 MHZ, CDCl3) δ 7.30 (dd, J=24.7, 4.8 Hz, 6H), 4.56 (s, 2H), 3.93-3.41 (m, 21H).

### Synthesis of the Intermediate LE-1-JG5060-5

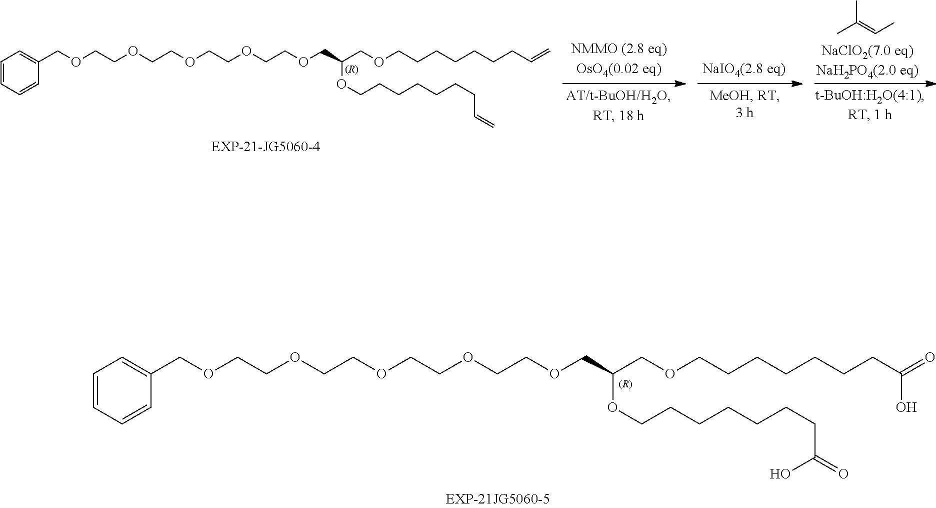

A solution of (R)-16-(non-8-en-1-yloxy)-1-phenyl-2,5,8,11,14,18-hexaoxaheptacos-26-ene (25 g, 41.8 mmol), 4-methyl-4-oxido-morpholin-4-ium (13.5 g, 115 mmol) and tetraoxoosmium (0.2 g, 0.84 mmol) in acetone (200 mL), t-BuOH (40 mL), and H₂O (20 mL) was stirred at room temperature for 12 h. TLC (DCM:MeOH=10:1, Rf: ˜0.5, Phosphomolybdic acid).indicated that all starting materials was consumed. After the mixture was concentrated, the residue was dissolved in EA and H₂O. The organic solution was separated, washed with brine, and dried Na2SO4. Evaporation of the solvent gave a crude glycol. A mixture of the crude glycol and NalO₄ (25.3 g, 118 mmol) in MeOH (250 mL) was stirred at room temperature for 3 h before concentration of the reaction mixture. EA (200 mL) and H₂O (200 mL) were added to the residue. The organic solution was separated, washed with brine, and dried Na2SO4. Concentrated gave a crude aldehyde. To a solution of the crude aldehyde, 2-methyl-2butene (180 mL), and NaH₂PO₄*2H₂O (9.4 g, 78.4 mmol), in t-BuOH (200 mL) and H₂O (50 mL) was added NaClO₂ (24.7 g, 274.4 mmol). The solution was stirred at room temperature for 1 h, and then EA was added. The organic solution was separated, washed with brine, and dried Na2SO4. Concentrated followed by silica gel column chromatography MeOH in DCM (8%) to give (R)-16-((7-carboxyheptyl)oxy)-1-phenyl-2,5,8,11,14,18-hexaoxahexacosan-26-oic acid (7.0 g, 36.7% yield) as colorless oil.

1H NMR (400 MHZ, CDCl3) δ 7.39-7.20 (m, 5H), 4.57 (s, 2H), 3.77-3.35 (m, 25H), 2.32 (t, J=7.4 Hz, 4H), 1.71-1.46 (m, 8H), 1.32 (s, 12H).

### Synthesis of the Intermediate LE-1-JG5060-6

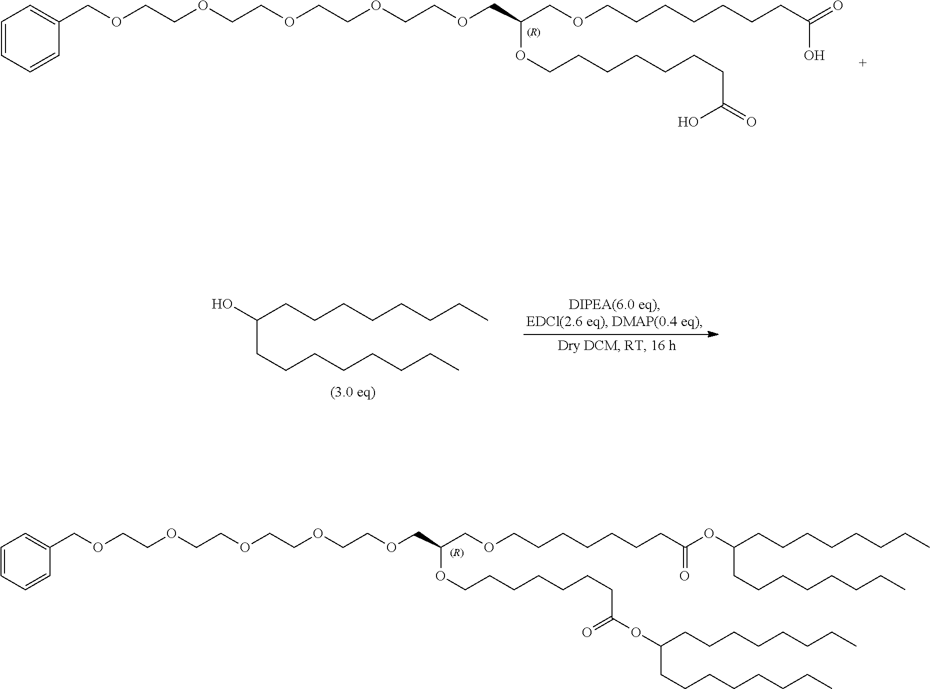

A mixture of (R)-16-((7-carboxyheptyl)oxy)-1-phenyl-2,5,8,11,14,18-hexaoxahexacosan-26-oic acid (5 g, 7.78 mmol), heptadecan-9-ol (7.98 g, 31.1 mmol), 3-(((ethylimino) methylene) amino)-N,N-dimethylpropan-1-amine hydrochloride (494 mg, 4.04 mmol), N,N-dimethylpyridin-4-amine (0.38 g, 3.11 mmol) and N-ethyl-N-isopropyl-propan-2-amine (6.03 g, 46.7 mmol) in DCM (50 mL) was stirred at room temperature 16 hrs. TLC (DCM: MeOH=10:1, Rf: ˜0.6, Phosphomolybdic acid) indicated that all starting materials was consumed. The reaction was poured into dilute HCl (50 mL, 1 mol/L) and extracted with dichloromethane (50 ml×3). The organic layers were combined, washed with brine (100 mL), dried over anhydrous sodium sulfate, filtered and concentrated. The residue, combined with above batch, was purified through flash chromatography eluted with 30% PE in EA to give of heptadecan-9-yl (R)-16-((8-(heptadecan-9-yloxy)-8-oxooctyl)oxy)-1-phenyl-2,5,8,11,14,18-hexaoxahexacosan-26-oate (1.0 g, 10.9% yield) as pale yellow oil.

1H NMR (400 MHZ, CDCl3) δ 7.36-7.27 (m, 4H), 4.90-4.79 (m, 2H), 4.57 (s, 2H), 3.75-3.35 (m, 25H), 2.27 (t, J=7.0 Hz, 4H), 1.66-1.42 (m, 18H), 1.28 (d, J=22.8 Hz, 60H), 0.88 (t, J=6.8 Hz, 12H).

### Synthesis of the Intermediate LE-1-JG5060-7

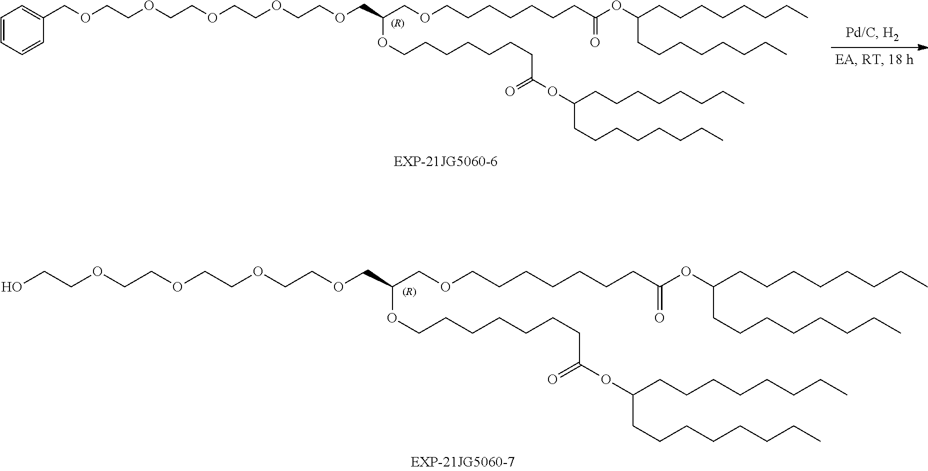

A solution of 1-octylnonyl heptadecan-9-yl (R)-16-((8-(heptadecan-9-yloxy)-8-oxooctyl)oxy)-1-phenyl-2,5,8,11,14,18-hexaoxahexacosan-26-oate (1.0 g, 0.893 mmol) was dissolved in 15 mL ethyl acetate. To the mixture was added Pd—C(400 mg, 10% on activated wood carbon, 50% water wet) in a 50 ml glass bottle and stirred for overnight at room temperature under H₂ balloon. TLC (DCM:MeOH=10:1), Rf: ˜ 0.5, Phosphomolybdic acid) showed the raw material disappeared. The mixture was then filtered and the filtrate was concentrated to give heptadecan-9-yl (R)-14-((8-(heptadecan-9-yloxy)-8-oxooctyl)oxy)-1-hydroxy-3,6,9, 12,16-pentaoxatetracosan-24-oate (0.8 g, 87.0% yield) as pale oil.

1H NMR (400 MHZ, CDCl3) δ 4.86 (p, J=6.2 Hz, 2H), 3.78-3.31 (m, 25H), 2.28 (dd, J=11.1, 3.9 Hz, 4H), 1.56 (ddd, J=22.7, 14.6, 6.1 Hz, 16H), 1.38-1.15 (m, 60H), 0.88 (t, J=6.8 Hz, 12H).

### Synthesis of the Compound XXXIII

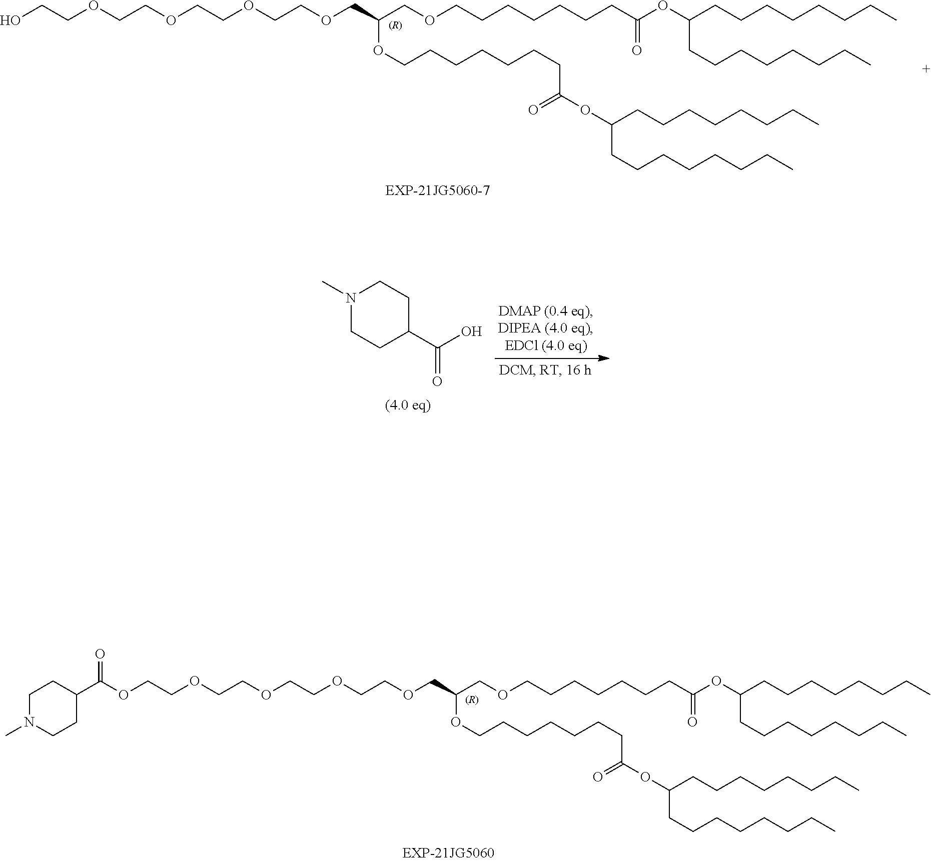

A mixture of heptadecan-9-yl (R)-14-((8-(heptadecan-9-yloxy)-8-oxooctyl)oxy)-1-hydroxy-3,6,9,12,16-pentaoxatetracosan-24-oate (800 mg, 0.77 mmol), 3-(((ethylimino) methylene) amino)-N,N-dimethylpropan-1-amine hydrochloride (387 mg, 2.02 mmol), N,N-dimethylpyridin-4-amine (19 mg, 0.155 mmol) and N-ethyl-N-isopropyl-propan-2-amine (0.603 g, 4.66 mmol) in 20 mL DCM was stirred at room temperature 16 hrs. TLC (DCM: MeOH=10:1, Rf: ˜ 0.4, Phosphomolybdic acid) showed the raw material disappeared. The reaction was poured into brine (10 mL) and extracted with dichloromethane (10 ml×3). The organic layers were combined, dried over anhydrous sodium sulfate, filtered, and concentrated. The residue, combined with above batch, Then purified by column chromatography with DCM in CH₃OH (0-10%) to give (R)-14-((8-(heptadecan-9-yloxy)-8-oxooctyl)oxy)-26-octyl-24-oxo-3,6,9,12,16,25-hexaoxatetratriacontyl 1-methylpiperidine-4-carboxylate (475 mg, 52.9%) as pale yellow oil.

1H NMR (400 MHZ, CDCl3) δ 4.92-4.78 (m, 2H), 4.28-4.18 (m, 2H), 3.70-3.42 (m, 23H), 2.85 (d, J=11.2 Hz, 2H), 2.37-2.23 (m, 8H), 1.82 (d, J=10.8 Hz, 2H), 1.64-1.46 (m, 18H), 1.38-1.21 (m, 63H), 0.88 (t, J=6.8 Hz, 12H).

### Example 26: Synthesis of bis(2-butyloctyl) 10-[3-[2-[3-[2-[2-[(1-methylpiperidine-4-carbonyl) amino]ethoxy]ethoxy]propanoylamino]ethyl disulfanyl] propanoyl-nonyl-amino] nonadecanedioate (compound XXXIV)

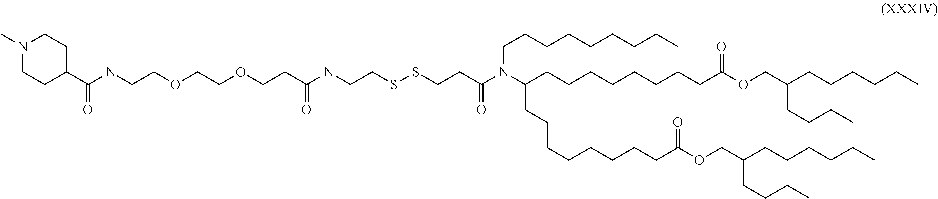 [SMILES: CCCCCCCCCN(C(=O)CCSSCCNC(=O)CCOCCOCCNC(=O)C1CCN(C)CC1)C(CCCCCCCCC(=O)OCC(CCCC)CCCCCC)CCCCCCCCC(=O)OCC(CCCC)CCCCCC]

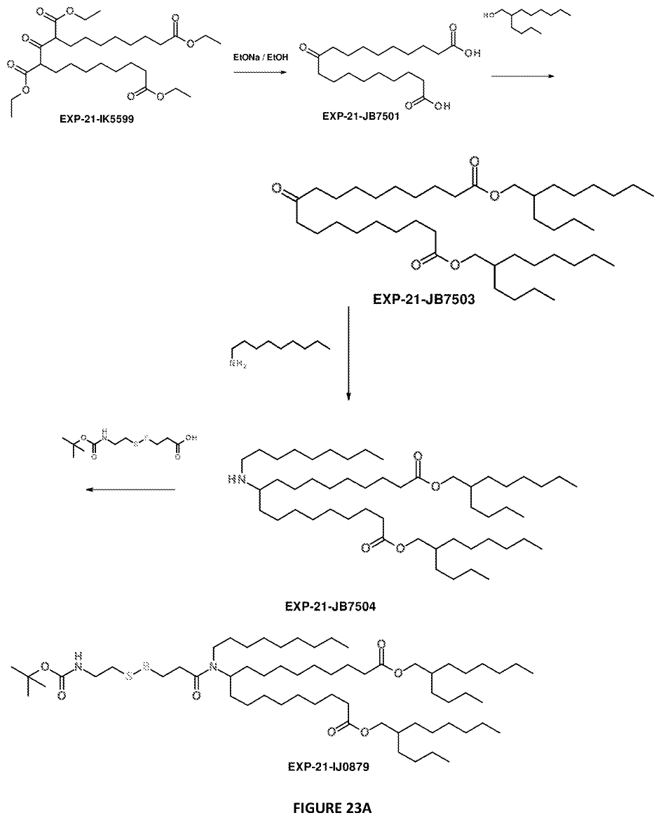

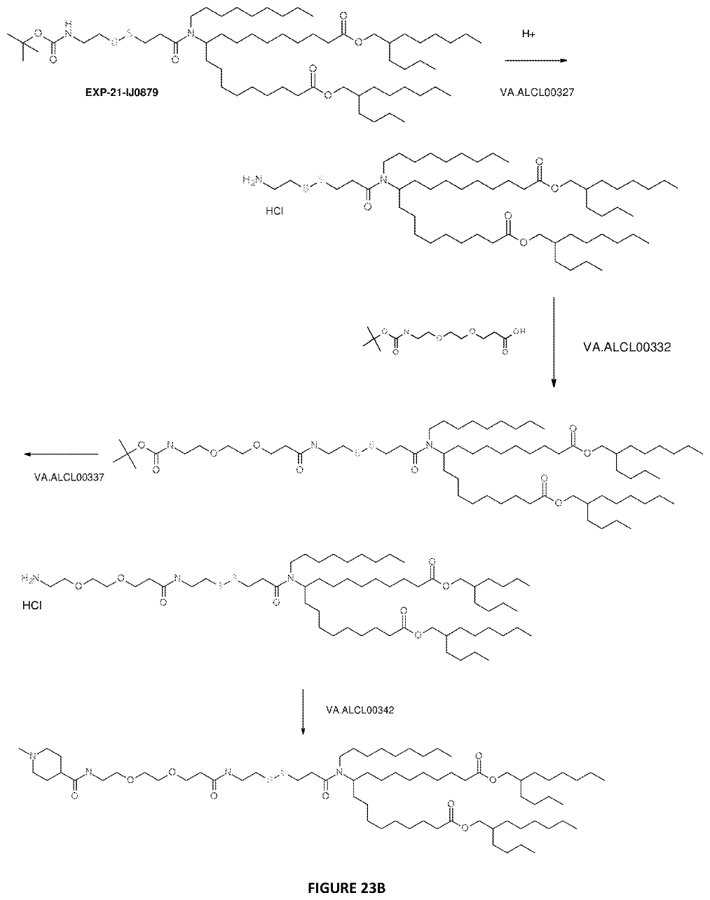

The compound XXXIV is prepared according to the schema of synthesis of FIGS. 23A and 23B.

### Synthesis of the Intermediate EXP-21-IK5599

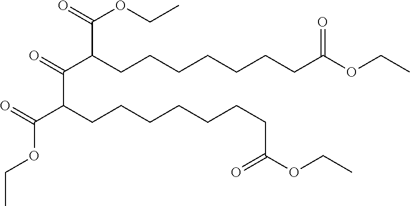 [SMILES: CCOC(=O)CCCCCCCC(C(=O)OCC)C(=O)C(CCCCCCCC(=O)OCC)C(=O)OCC]

A mixture of diethyl 3-oxopentanedioate (20 g) and a 20% sodium ethoxide-ethanol solution (33.5 g) were stirred at 80° C. for 20 minutes, ethyl 8-bromooctanoate (25 g) was then added thereto, and the mixture was stirred for 4 hours. A 20% sodium ethoxide-ethanol solution (33.5 g) was added to the reaction mixture, the reaction mixture was stirred for 5 minutes, ethyl 8-bromooctanoate (25 g) was then added there to, and the mixture was stirred for 3 hours. The reaction mixture was cooled to room temperature, hexane and a 20% aqueous ammonium chloride solution (110 mL) were then added there to the organic layer was separated, and the solvent was distilled away under reduced pressure, thereby obtaining tetraethyl 9-oxoheptadecane-1,8,10,17-tetracarboxylate (51.5 g) as a crude product.

LCMS: Rt=2.194

### Synthesis of the Intermediate EXP-21-JB7501

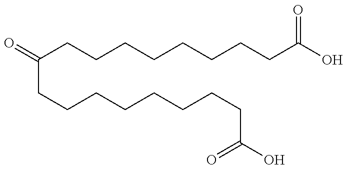 [SMILES: O=C(O)CCCCCCCCC(=O)CCCCCCCCC(=O)O]

A mixture of the obtained tetraethyl 9-oxoheptadecane-1,8,10,17-tetracarboxylate (25 g), acetic acid (40 mL), and a 30% aqueous hydrochloric acid solution (80 mL) were stirred at 115° C. for 6 hours. The reaction mixture was cooled to room temperature, the solvent was then distilled away under reduced pressure, and water and acetone were added to the residue. Solids were collected by filtration, washed with water and acetone, and then dried under reduced pressure, thereby obtaining 10-oxononane decanedioic acid (0.6 g) as white solids.

1H NMR (400 MHZ, DMSO) δ 11.97 (s, 2H), 2.38 (t, J=7.3 Hz, 4H), 2.18 (t, J=7.4 Hz, 4H), 1.54-1.35 (m, 8H), 1.23 (s, 16H).

### Synthesis of the Intermediate EXP-21-JB7503

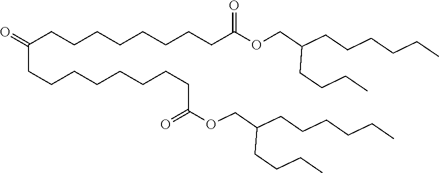 [SMILES: CCCCCCC(CCCC)COC(=O)CCCCCCCCC(=O)CCCCCCCCC(=O)OCC(CCCC)CCCCCC]

1-Ethyl-3-(3-dimethylaminopropyl) carbodiimide hydrochloride (10.1 g, 52.6 mmol) was added to a mixture of 10-oxononane decanedioic acid (7.35 g, 39.4 mmol), 2-butyloctan-1-ol (9 g, 26.3 mmol), triethylamine (11 ml-dimethylaminopyridine (321,0.26 mmol) and dichloromethane (500 mL), and the mixture was stirred at room temperature for 2 days. A 10% aqueous potassium hydrogen sulfate solution (1200 mL), hexane (600 mL), and ethyl acetate (600 mL) were added to the reaction mixture, the organic layer was separated and then dried over anhydrous sodium sulfate, and the solvent was distilled away under reduced pressure. The obtained residue was purified by silica gel column chromatography (ethyl acetate-hexane), thereby obtaining bis(2-butyloctyl) IO-oxononane decanedioate (10 g, 56%) as a colorless oily substance.

1H NMR (400 MHZ, CDCl3) δ 3.97 (d, J=5.8 Hz, 4H), 2.37 (t, J=7.5 Hz, 4H), 2.29 (t, J=7.5 Hz, 4H), 1.71-1.43 (m, 11H), 1.28 (d, J=1.2 Hz, 49H), 0.89 (td,J=6.6, 4.1 Hz, 12H).

### Synthesis of the Intermediate EXP-21-JB7504

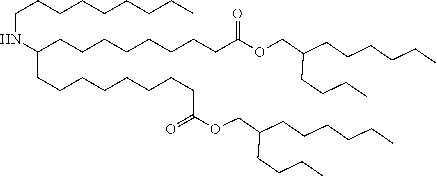 [SMILES: CCCCCCCCCNC(CCCCCCCCC(=O)OCC(CCCC)CCCCCC)CCCCCCCCC(=O)OCC(CCCC)CCCCCC]

A solution of the bis(2-butyloctyl) 10-oxononadecanedioate (6 g, 8.84 mmol) and 1-decylamine (2.53 g, 13.2 mmol) in DCE (10 mL) was stirred at room temperature for 15 minutes, followed by addition of sodium triacetoxyborohydride (2.79 g, 13.2 mmol) and acetic acid (0.828 mL). After the mixture was stirred at room temperature for 2 days, the reaction mixture was concentrated. The residue was diluted with hexane and washed with diluted NaOH, saturated NaHCO₃ and brine. The organic phase was separated, dried over sodium sulfate and concentrated. The crude product was purified by column chromatography on silica gel (petroleum ether/EtOAc, 95:5:0 to 80:20:1) to give bis(2-butyloctyl) 10-(nonylamino)nonadecanedioate (2.5 g, 35% yield) as colorless oil.

1H NMR (500 MHZ, CDCl3) δ 3.97 (d, J=5.8 Hz, 4H), 2.54 (t, J=7.3 Hz, 2H), 2.47-2.37 (m, 1H), 2.30 (t, J=7.5 Hz, 4H), 1.66-1.58 (m, 6H), 1.45 (d, J=6.7 Hz, 2H), 1.31 (dd, J=32.7, 5.2 Hz, 70H), 0.94-0.79 (m, 16H).

### Synthesis of the Intermediate EXP-21-IJ0879

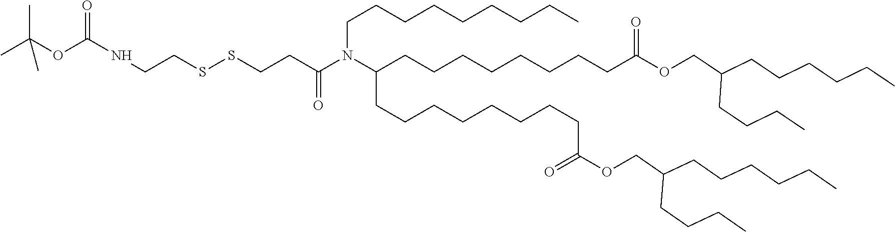 [SMILES: CCCCCCCCCN(C(=O)CCSSCCNC(=O)OC(C)(C)C)C(CCCCCCCCC(=O)OCC(CCCC)CCCCCC)CCCCCCCCC(=O)OCC(CCCC)CCCCCC]

To the of 3-[2-(tert-solution butoxycarbonylamino) ethyldisulfanyl] propanoic acid (2.18 g, 7.75 mmol) and bis(2-butyloctyl) 10-(nonylamino)nonadecanedioate (2.5 g, 3.10 mol) in DCM (30 mL) were added HATU (3.54 g, 7.75 mmol) and DIPEA (1.20 g, 7.75 mol). The mixture was stirred at room temperature for 18 h. The reaction was quenched with water (50 mL) then extracted with ethyl acetate (2*100 mL). The organic layer was washed with brine, dried over sodium sulfate, filtered, and concentrated. The residue was purified by flash chromatography column eluted with 0% to 10% EA in PE to give bis(2-butyloctyl) 10-[3-[2-(tert-butoxycarbonylamino) ethyldisulfanyl] propanoyl-nonyl-amino] nonadecanedioate as colorless oil (2.03 g, 61.2% yield).

LCMS: no LC shows

1H NMR (400 MHZ, CDCl3) δ 5.11 (d, J=25.7 Hz, 1H), 3.97 (d, J=5.8 Hz, 4H), 3.58 (dd, J=25.4, 18.4 Hz, 1H), 3.46 (d, J=6.0 Hz, 2H), 3.13-2.92 (m, 4H), 2.89-2.60 (m, 4H), 2.33-2.23 (m, 4H), 1.61 (d, J=3.0 Hz, 6H), 1.44 (s, 14H), 1.28 (s, 66H), 0.94-0.81 (m, 15H).

### Synthesis of bis(2-butyloctyl) 10-[3-(2-aminoethyldisulfanyl) propanoyl-nonyl-amino] nonadecanedioate; hydrochloride

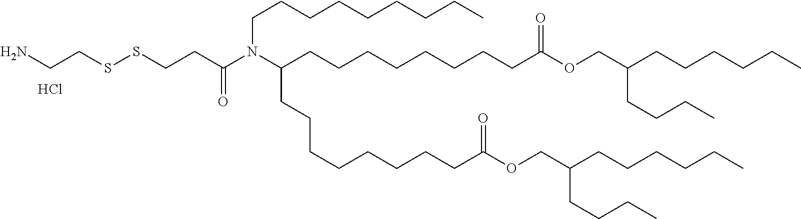 [SMILES: CCCCCCCCCN(C(=O)CCSSCCN)C(CCCCCCCCC(=O)OCC(CCCC)CCCCCC)CCCCCCCCC(=O)OCC(CCCC)CCCCCC ; Cl]

In a 5 mL microwave tube on an orbital shaker, intermediate EXP-21-IJ0879 and dichloromethane (4 mL) are loaded. 4N hydrochloric dioxane (2.337 mmol, 0.5842 mL) is added and stirred at room temperature overnight.

TLC DCM 90 MeOH 9.5, H₂O 0.5: reaction complete. Concentrate the MR to dryness Then, purify by chromate-flash: Merck silica cartridge of 10 g, Eluent: DCM/MeOH gradian from 100 to 80/20 in 35 min there by obtaining 180 mg, 74% bis(2-butyloctyl) 10-[3-(2-aminoethyldisulfanyl) propanoyl-nonyl-amino] nonadecanedioate hydrochloride as a colorless oil.

### Synthesis of bis(2-butyloctyl) 10-[3-[2-[3-[2-[2-(tert-butoxycarbonylamino) ethoxy] ethoxy]propanoylamino]ethyldisulfanyl] propanoyl-nonyl-amino] nonadecanedioate

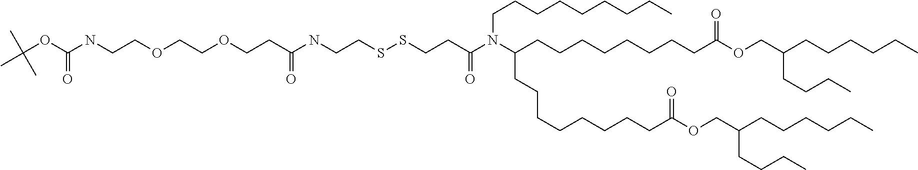 [SMILES: CCCCCCCCCN(C(=O)CCSSCCNC(=O)CCOCCOCCNC(=O)OC(C)(C)C)C(CCCCCCCCC(=O)OCC(CCCC)CCCCCC)CCCCCCCCC(=O)OCC(CCCC)CCCCCC]

In a 10 mL microwave tube on an orbital shaker table, N-Boc-3-[2-(2-amino (ethoxy) ethoxy] propionic acid, and 2 mL of DCM are charged. HATU (0.2684 mmol, 102.054 mg) and N,N-Diisopropylethylamine (0.5367 mmol, 0.096 mL, 69.3648 mg) are added. Stirring at room temperature overnight.

Concentrate the MR to dryness then purify by chromate-flash: 100 g Merck silica cartridge Eluent: DCM/MeOH/H₂O, Gradient from 100/0/0 to 80/10/1 in 35 min there by obtaining 200 mg, 90% of bis(2-butyloctyl) 10-[3-[2-[3-[2-[2-(tert-butoxycarbonylamino) ethoxy]ethoxy]propanoylamino]ethyldisulfanyl] propanoyl-nonyl-amino] nonadecanedioate as a colorless oil.

### Synthesis of bis(2-butyloctyl) 10-[3-[2-[3-[2-(2-aminoethoxy) ethoxy]propanoylamino] ethyldisulfanyl] propanoyl-nonyl-amino] nonadecanedioate;hydrochloride

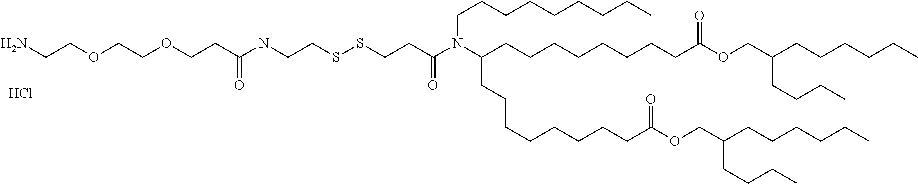 [SMILES: Cl ; CCCCCCCCCN(C(=O)CCSSCCNC(=O)CCOCCOCCN)C(CCCCCCCCC(=O)OCC(CCCC)CCCCCC)CCCCCCCCC(=O)OCC(CCCC)CCCCCC]

In a 5 mL microwave tube on an orbital shaker, previous intermediate bis(2-butyloctyl) 10-[3-[2-[3-[2-[2-(tert-butoxycarbonylamino) ethoxy] ethoxy]propanoylamino]ethyldisulfanyl] propanoyl-nonyl-amino] nonadecanedioate and dichloromethane (4 mL) are loaded. 4N hydrochloric dioxane (1.4647 mmol, 0.3662 mL) is added and stirred at room temperature overnight. TLC DCM 90 MeOH 9.5, H₂O 0.5: reaction complete. Concentrate the MR to dryness.

Purification by flash chromatography: 10 g Merck silica cartridge, Eluent: DCM/MeOH, guardian from 100 to 80/20 in 35 min, there by obtaining 115 mg, 65% bis(2-butyloctyl) 10-[3-[2-[3-[2-(2-aminoethoxy) ethoxy]propanoylamino]ethyldisulfanyl] propanoyl-nonyl-amino] nonadecanedioate;hydrochloride as a colorless oil.

### Synthesis of Compound XXXIV

In a 5 mL microwave tube on an orbital shaker, and 3 mL of peptide DMF are loaded. HATU (0.1382 mmol, 52.5481 mg) and N,N-Diisopropylethylamine (0.3948 mmol, 0.071 mL, 51.0252 mg) are added, then add the 1-methylpiperidine-4-carboxylic acid (19,788 mg, 0,1382 mmol). Stirring at room temperature overnight. Concentrate the MR to dryness. Then purify by chromate-flash: 10 g Merck silica cartridge, Eluent: DCM/MeOH, Gradient from 100/0 to 80/20 in 35 min there by obtaining 33 mg, 26% bis(2-butyloctyl) 10-[3-[2-[3-[2-(2-aminoethoxy) ethoxy]propanoylamino]ethyldisulfanyl] propanoyl-nonyl-amino] nonadecanedioate;hydrochloride.

1H NMR (400 MHZ, DMSO-d6, 100° C.) δ ppm 0.79-0.92 (m, 15H), 1.14-1.76 (m, 78H), 1.99-2.17 (m, 2H), 2.20-2.28 (m, 9H), 2.29-2.35 (m, 3H), 2.60-2.70 (m, 2H), 2.79 (t, J=7 Hz, 2H), 2.81-3.13 (m partially hidden, 7H), 3.17-3.24 (m, 2H), 3.29-3.38 (m, 2H), 3.42 (t, J-6 Hz, 2H), 3.49 (s, 4H), 3.62 (t, J=7 Hz, 2H), 3.92 (d, J=6 Hz, 4H), 7.22-7.34 (m, 1H), 7.52-7.67 (m, 1H)

[M+H]+, rt=6.54 min.

### Example 27: Synthesis of 2-[2-[2-[2-[2,3-bis [6-(2-hexyldecanoyloxy) hexoxy] propoxy] ethoxy]ethoxy] ethoxy]ethyl-1,4-dimethylpiperidine-4-carboxylate (compound XXXV)

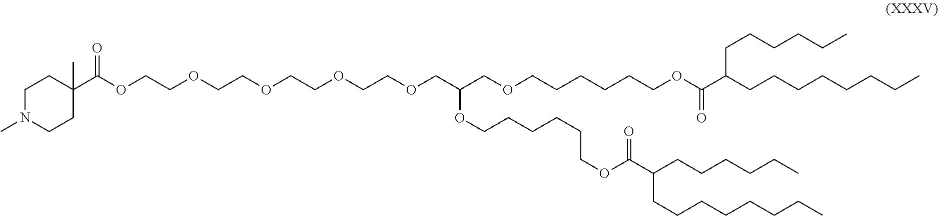 [SMILES: CCCCCCCCC(CCCCCC)C(=O)OCCCCCCOCC(COCCOCCOCCOCCOC(=O)C1(C)CCN(C)CC1)OCCCCCCOC(=O)C(CCCCCC)CCCCCCCC]

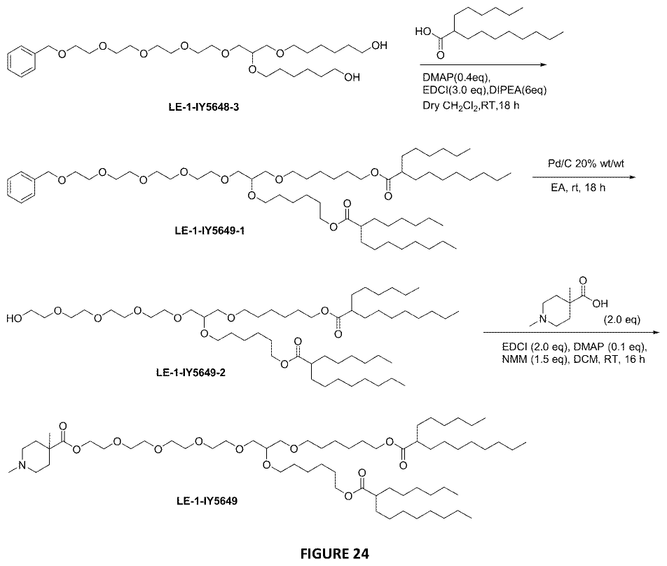

The compound XXXV is prepared according to the schema of synthesis of FIG. 24.

### Synthesis of the Intermediate LE-1-IY5649-1

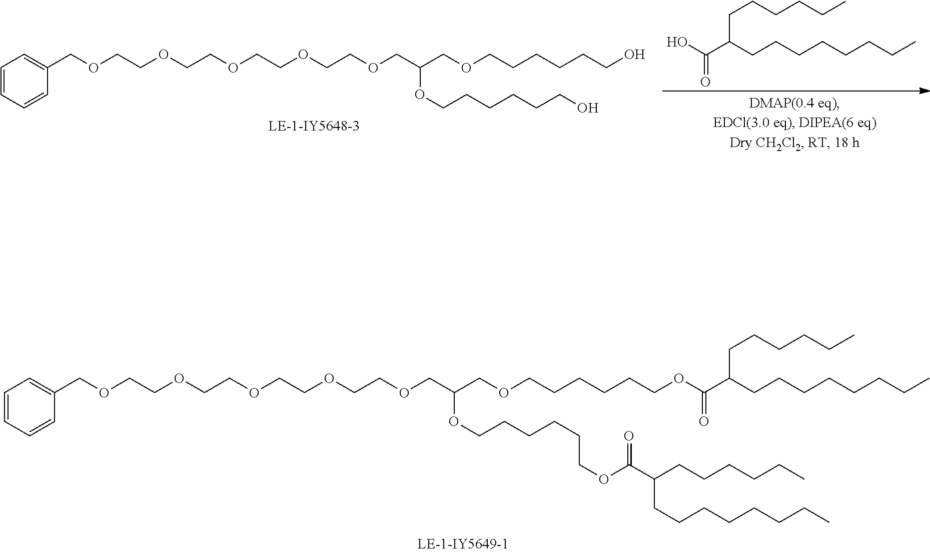 [SMILES: OCCCCCCOCC(COCCOCCOCCOCCOCc1ccccc1)OCCCCCCO ; CCCCCCCCC(CCCCCC)C(=O)O ; CCCCCCCCC(CCCCCC)C(=O)OCCCCCCOCC(COCCOCCOCCOCCOCc1ccccc1)OCCCCCCOC(=O)C(CCCCCC)CCCCCCCC]

To the solution of 16-((6-hydroxyhexyl)oxy)-1-phenyl-2,5,8,11,14,18-hexaoxatetracosan-24-ol (1.1 g, 1.97 mmol) and 2-hexyldecanoic acid (1.51 g, 5.91 mmol) in dry dichloromethane (20 mL) were added DIPEA (1.53 g, 11.8 mmol), DMAP (0.096 g, 0.78 mmol) and EDCl (1.13 g, 5.91 mmol) under ice bath. The mixture was stirred at room temperature for 18 h. The reaction was washed with 0.1 M HCl and NaHCO₃ and brine. The organic layer was dried over sodium sulfate, filtered, and concentrated. The residue was purified by flash chromatography eluted with 0% to 5% CH₃OH in DCM to give6-[3-[2-[2-[2-(2-benzyloxyethoxy) ethoxy]ethoxy] ethoxy]-2-[6-(2-hexyldecanoyloxy) hexoxy] propoxy] hexyl 2-hexyldecanoate (1.3 g, 1.26 mmol, 63.8% yield) as a colorless oil.

1H NMR (400 MHZ, CDCl3) δ 7.40-7.27 (m, 5H), 4.57 (s, 2H), 4.06 (td, J=6.6, 1.3 Hz, 4H), 3.90-3.23 (m, 25H), 2.35-2.23 (m, 2H), 1.59 (dd, J=16.6, 5.1 Hz, 12H), 1.45-1.20 (m, 54H), 0.87 (dd, J=6.9, 6.1 Hz, 12H).

### Synthesis of the Intermediate LE-1-IY5649-2

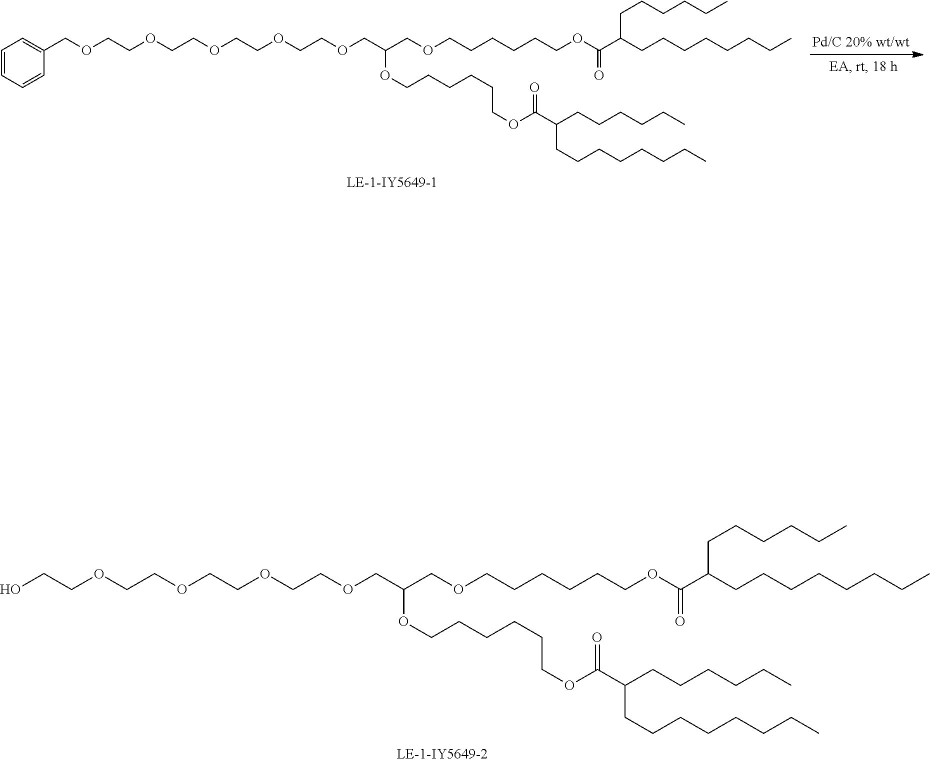 [SMILES: CCCCCCCCC(CCCCCC)C(=O)OCCCCCCOCC(COCCOCCOCCOCCOCc1ccccc1)OCCCCCCOC(=O)C(CCCCCC)CCCCCCCC ; CCCCCCCCC(CCCCCC)C(=O)OCCCCCCOCC(COCCOCCOCCOCCO)OCCCCCCOC(=O)C(CCCCCC)CCCCCCCC]

A solution of 6-[3-[2-[2-[2-(2-benzyloxyethoxy) ethoxy]ethoxy] ethoxy]-2-[6-(2-hexyldecanoyloxy) hexoxy] propoxy] hexyl 2-hexyldecanoate (1.3 g, 1.26 mmol) in EtOAc (30 mL) was added Pd/C (300 mg) and the reaction was backfilled with H₂ for 3 times. The reaction was stirred overnight at room temperature under H₂ atmosphere. TLC (5% CH₃OH in DCM) shows that the reaction was finished. The reaction mixture was filtered through celite and the celite washed with EtOAc. The organic phase was concentrated under vacuum to give 6-[2-[6-(2-hexyldecanoyloxy) hexoxy]-3-[2-[2-[2-(2-hydroxyethoxy) ethoxy]ethoxy] ethoxy] propoxylhexyl 2-hexyldecanoate as colorless.

1H NMR (400 MHZ, CDCl3) δ 4.10-3.98 (m, 4H), 3.76-3.34 (m, 25H), 2.37-2.23 (m, 2H), 1.68-1.52 (m, 12H), 1.46-1.21 (m, 52H), 0.87 (t, J=6.5 Hz, 12H).

### Synthesis of Compound XXXV

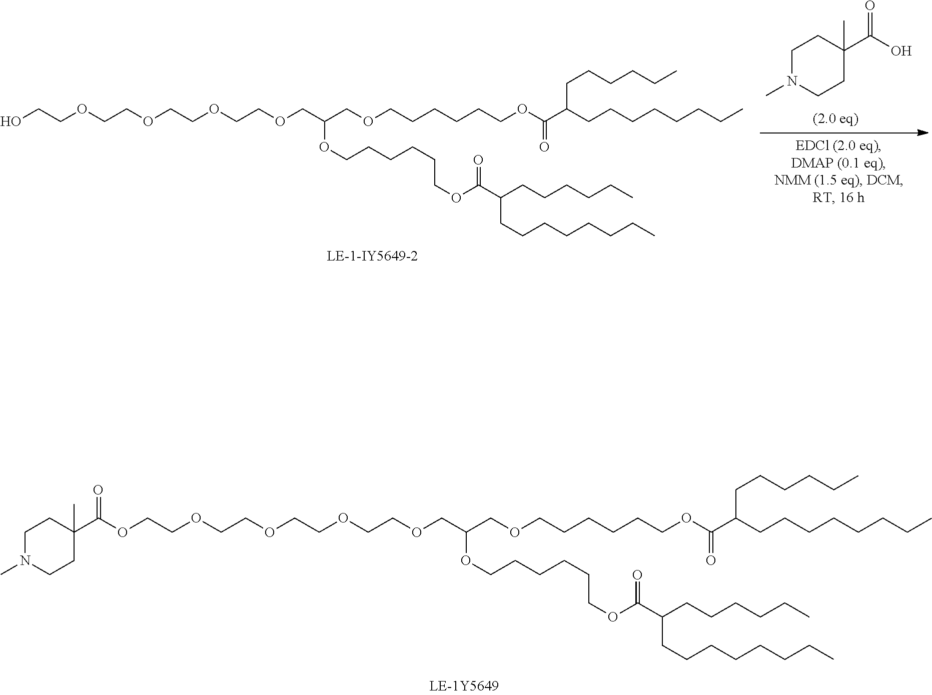

DIPEA (1.37 g, 10.6 mmol) was added to the solution of 1,4-dimethylpiperidine-4-carboxylic acid (0.24 g, 1.59 mmol) in dry dichloromethane (40 mL), stirred for 10 min until the solution was clear. Added 6-[2-[6-(2-hexyldecanoyloxy) hexoxy]-3-[2-[2-[2-(2-hydroxyethoxy) ethoxy]ethoxy] ethoxy] propoxy] hexyl 2-hexyldecanoate (1.0 g, 1.06 mmol) and DMAP (0.129 g, 1.06 mmol) and EDCl (1.01 g, 5.29 mmol) to the solution under ice bath. The mixture was stirred at room temperature for 18 h. The reaction was washed with Na₂CO₃ (30 mL) and brine. The organic layer was dried over sodium sulfate, filtered and concentrated. The residue was purified by flash chromatography eluted with 0% to 5% CH₃OH in DCM to give 2-[2-[2-[2-[2,3-bis [6-(2-hexyldecanoyloxy) hexoxy] propoxy] ethoxy]ethoxy] ethoxy]ethyl 1,4-dimethylpiperidine-4-carboxylate (0.538 g, 0.49 mmol, 46.9% yield) as a colorless oil.

1H NMR (400 MHZ, CDCl3) δ 4.32-4.19 (m, 2H), 4.06 (td, J=6.6, 1.3 Hz, 4H), 3.74-3.34 (m, 23H), 2.60 (d, J=11.2 Hz, 2H), 2.43-1.89 (m, 11H), 1.69-1.22 (m, 66H), 1.19 (s, 3H), 0.87 (t, J=6.5 Hz, 12H).

### Example 28: Synthesis of 2-[2-[2-[2-[2,3-bis [8-(1-hexylnonoxy)-8-oxo-octoxy] propoxy] ethoxy]ethoxy] ethoxy]ethyl 1,4-dimethylpiperidine-4-carboxylate (compound XXXVI)

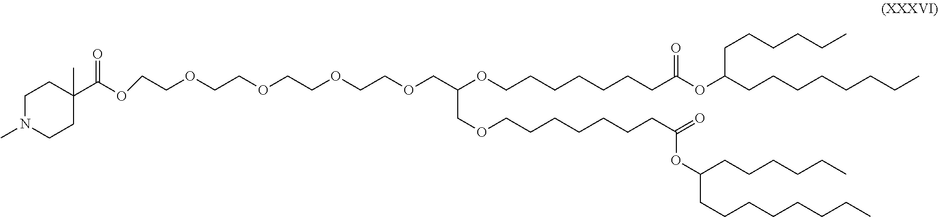 [SMILES: CCCCCCCCC(CCCCCC)OC(=O)CCCCCCCOCC(COCCOCCOCCOCCOC(=O)C1(C)CCN(C)CC1)OCCCCCCCC(=O)OC(CCCCCC)CCCCCCCC]

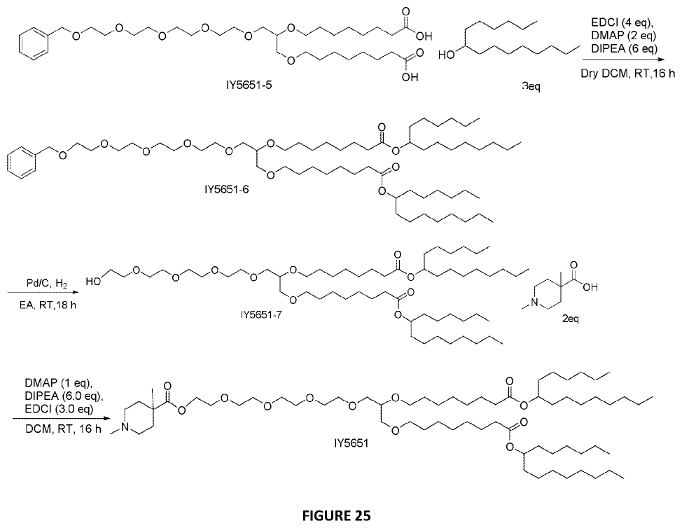

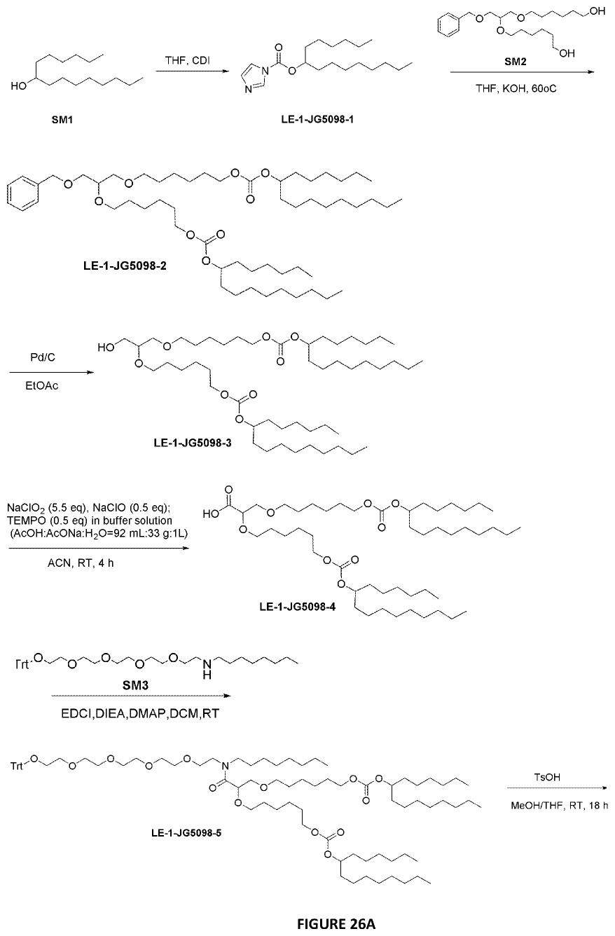

The compound XXXVI is prepared according to the schema of synthesis of FIG. 25.

### Synthesis of the Intermediate LE-1-IY5651-6

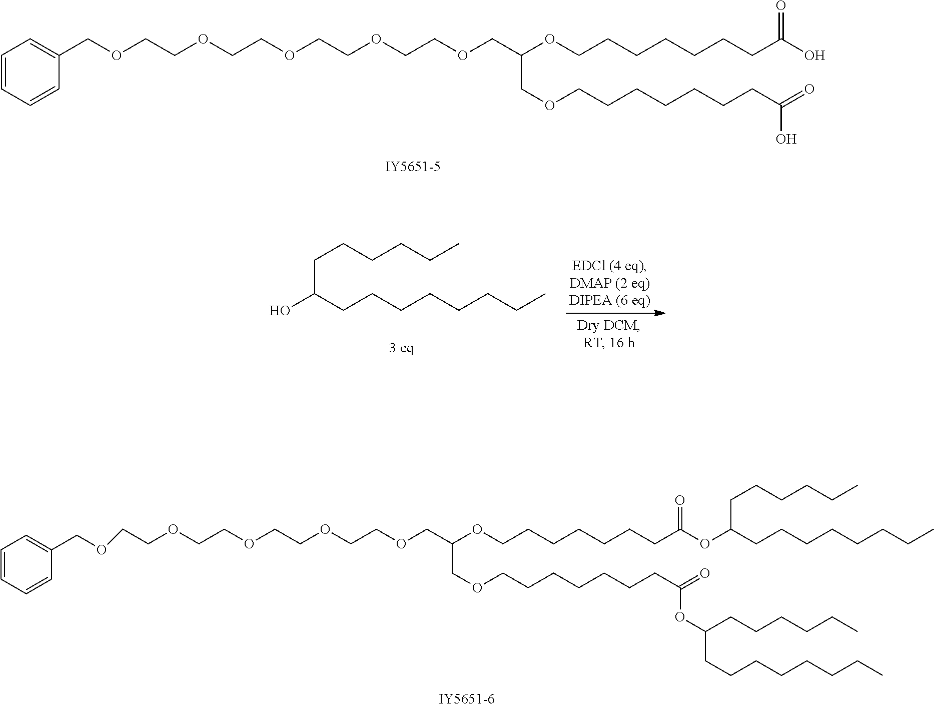 [SMILES: O=C(O)CCCCCCCOCC(COCCOCCOCCOCCOCc1ccccc1)OCCCCCCCC(=O)O ; CCCCCCCCC(O)CCCCCC ; CCCCCCCCC(CCCCCC)OC(=O)CCCCCCCOCC(COCCOCCOCCOCCOCc1ccccc1)OCCCCCCCC(=O)OC(CCCCCC)CCCCCCCC]

To a solution of 8-[3-[2-[2-[2-(2-benzyloxyethoxy) ethoxy]ethoxy] ethoxy]-2-(7-carboxyheptoxy) propoxy] octanoic acid (1.0 g, 1.56 mmol) in DCM (20 ml) was added pentadecan-7-ol (1.07 g, 4.67 mmol), 3-(ethyliminomethyleneamino)-N,N-dimethyl-propan-1-amine;hydrochloride (1.19 g, 6.22 mmol), DMAP (190 mg, 1.56 mmol) and N-ethyl-N-isopropyl-propan-2-amine (1.01 g, 7.78 mmol). The mixture was stirred at 25° C. for 16 hr. Then the mixture was concentrated and dealt with DCM (50 ml), washed with water (50 ml×2), brine (50 ml) and dried over Na2SO4. The organic was concentrated and purified by flash chromatography column (0-40% EA in PE) to give 1-hexylnonyl 8-[3-[2-[2-[2-(2-benzyloxyethoxy) ethoxy]ethoxy] ethoxy]-2-[8-(1-hexylnonoxy)-8-oxo-octoxy] propoxy] octanoate (0.89 g, yield 53.8%) as a colorless oil.

1H NMR (400 MHZ, CDCl3) δ 7.40-7.26 (m, 11H), 4.95-4.72 (m, 2H), 4.57 (s, 2H), 3.76-3.32 (m, 21H), 2.27 (t, J=7.1 Hz, 3H), 1.50 (d, J=5.6 Hz, 11H), 1.28 (d, J=22.6 Hz, 45H), 0.88 (t, J=6.8 Hz, 12H).

### Synthesis of the Intermediate LE-1-IY5651-7

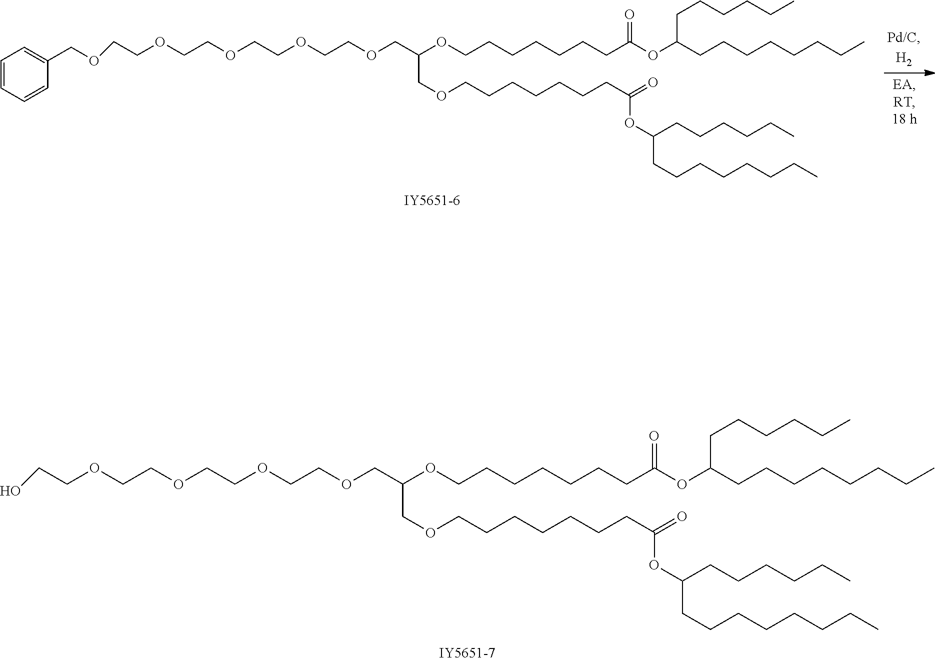 [SMILES: CCCCCCCCC(CCCCCC)OC(=O)CCCCCCCOCC(COCCOCCOCCOCCOCc1ccccc1)OCCCCCCCC(=O)OC(CCCCCC)CCCCCCCC ; CCCCCCCCC(CCCCCC)OC(=O)CCCCCCCOCC(COCCOCCOCCOCCO)OCCCCCCCC(=O)OC(CCCCCC)CCCCCCCC]

A solution of 1-hexylnonyl 8-[3-[2-[2-[2-(2-benzyloxyethoxy) ethoxy]ethoxy] ethoxy]-2-[8-(1-hexylnonoxy)-8-oxo-octoxy] propoxy] octanoate (0.89 g, 0.837 mmol) in EtOAc (20 mL) was purged for 10 minutes with N2 followed by addition of Pd/C (0.2 g) and the reaction continued purging with N2. The reaction was next evacuated under vacuum and backfilled with H_{2 3} times. The reaction was next stirred overnight at room temperature under an atmosphere of H₂. TLC (4% CH₃OH in DCM) shows that the reaction was finished. The slurry filtered through celite and the celite was rinsed with EtOAc several times. The combined organics were next concentrated under vacuum to give 1-hexylnonyl 8-[2-[8-(1-hexylnonoxy)-8-oxo-octoxy]-3-[2-[2-[2-(2-hydroxyethoxy) ethoxy]ethoxy] ethoxy] propoxy] octanoate (0.78 g, 95.8% yield) as colorless oil.

### Synthesis of the Compound XXXVI

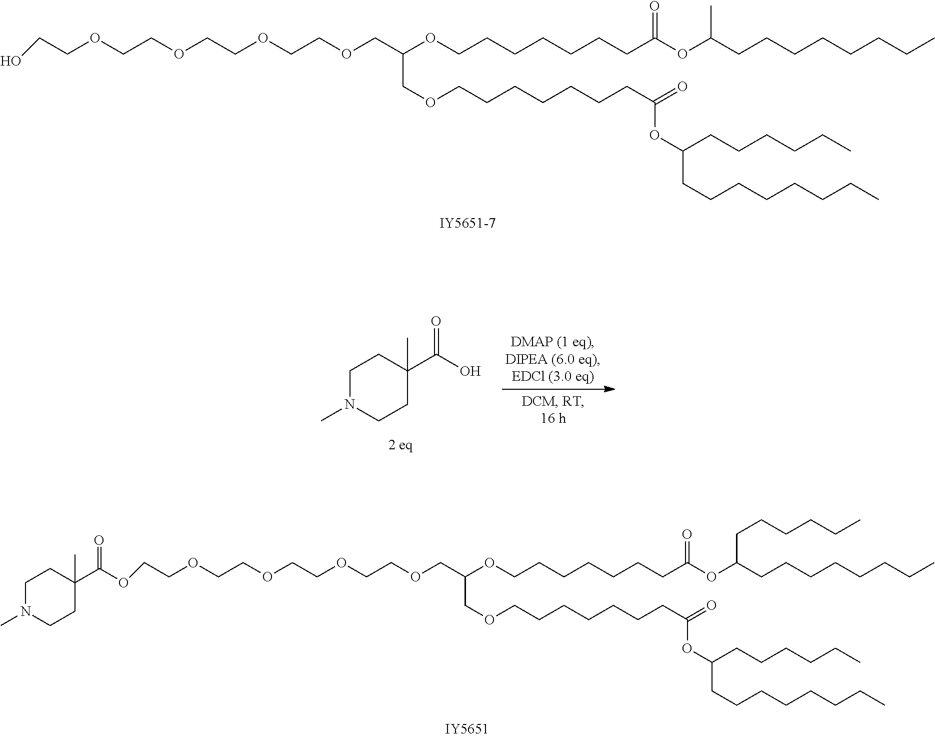

1-hexylnonyl 8-[2-[8-(1-hexylnonoxy)-8-oxo-octoxy]-3-[2-[2-[2-(2 hydroxyethoxy) ethoxy]ethoxy] ethoxy] propoxy] octanoate (0.78 g, 0.8 mmol) and 1,4-dimethylpiperidine-4-carboxylic acid (0.25 g, 1.6 mmol) in dry dichloromethane (10 mL) were added DIPEA (0.311 g, 2.4 mmol), DMAP (97 mg, 0.8 mmol) and followed by EDCl (0.311 g, 1.6 mmol). The mixture was stirred at room temperature for 16 h. The reaction was diluted with dichloromethane (50 mL) and washed with brine. The organic layer was dried over sodium sulfate, filtered, and concentrated. The residue was purified by flash chromatography eluted with 0% to 10% (6%) MeOH in DCM to give 2-[2-[2-[2-[2,3-bis [8-(1-hexylnonoxy)-8-oxo-octoxy] propoxy] ethoxy]ethoxy] ethoxy]ethyl 1,4-dimethylpiperidine-4-carboxylate (0.602 g, 67.5% yield) as light yellow oil.

1H NMR (400 MHZ, CDCl3) δ 4.95-4.75 (m, 2H), 4.33-4.22 (m, 2H), 3.83-3.33 (m, 21H), 2.86-2.60 (m, 1H), 2.45-2.11 (m, 9H), 1.54 (dd, J=28.2, 11.7 Hz, 22H), 1.26 (t, J=18.6 Hz, 52H), 0.88 (t, J=6.7 Hz, 12H).

### Example 29: Synthesis of 2-[2-[2-[2-[2-[2,3-bis [6-(1-hexylnonoxy carbonyloxy) hexoxy] propanoyl-octylamino]ethoxy]ethoxy] ethoxy]ethoxy]ethyl 1-methyl piperidine-4-carboxylate (compound XXXVII)

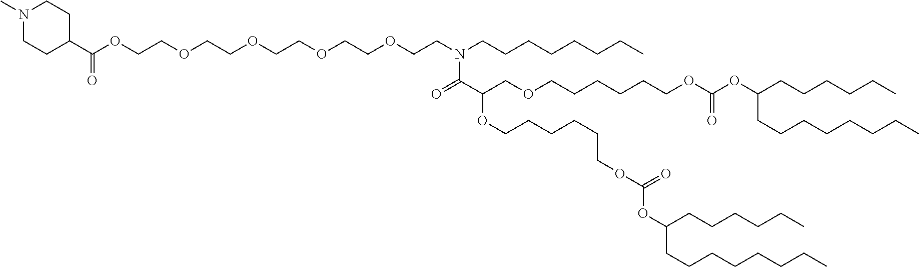 [SMILES: CCCCCCCCC(CCCCCC)OC(=O)OCCCCCCOCC(OCCCCCCOC(=O)OC(CCCCCC)CCCCCCCC)C(=O)N(CCCCCCCC)CCOCCOCCOCCOCCOC(=O)C1CCN(C)CC1]

### (XXXVII)

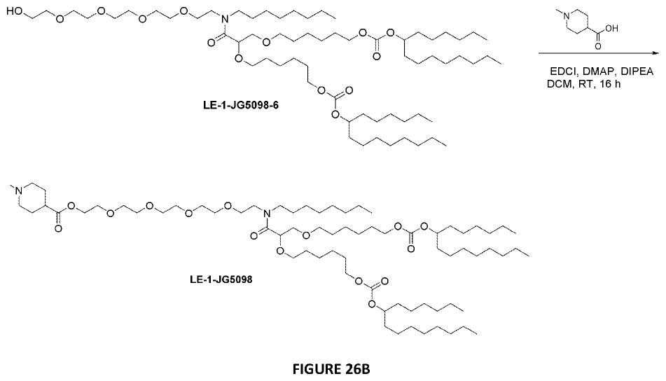

The compound XXXVII is prepared according to the schema of synthesis of FIGS. 26A and 26B.

### Synthesis of the Intermediate LE-1-JG5098-1

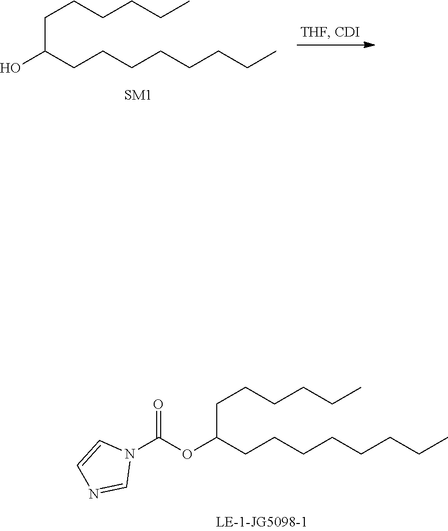 [SMILES: CCCCCCCCC(O)CCCCCC ; CCCCCCCCC(CCCCCC)OC(=O)n1ccnc1]

A solution of pentadecan-7-ol (1.5 g, 5.88 mmol), CDI (5.68 g, 35.0 mmol) in THF (30 mL) was stirred at room temperature for 16 h. Diluted with EA (200 mL), washed with brine (30 mL*3), dried with Na2SO4, filtered, purified by silica-gel (EA/PE=1/20), obtained 1-hexylnonyl imidazole-1-carboxylate (5.2 g, 16.1 mmol, 92.1% yield) as colorless oil.

LC-MS: 323.3 [M+H]+

### Synthesis of the Intermediate LE-1-JG5098-2

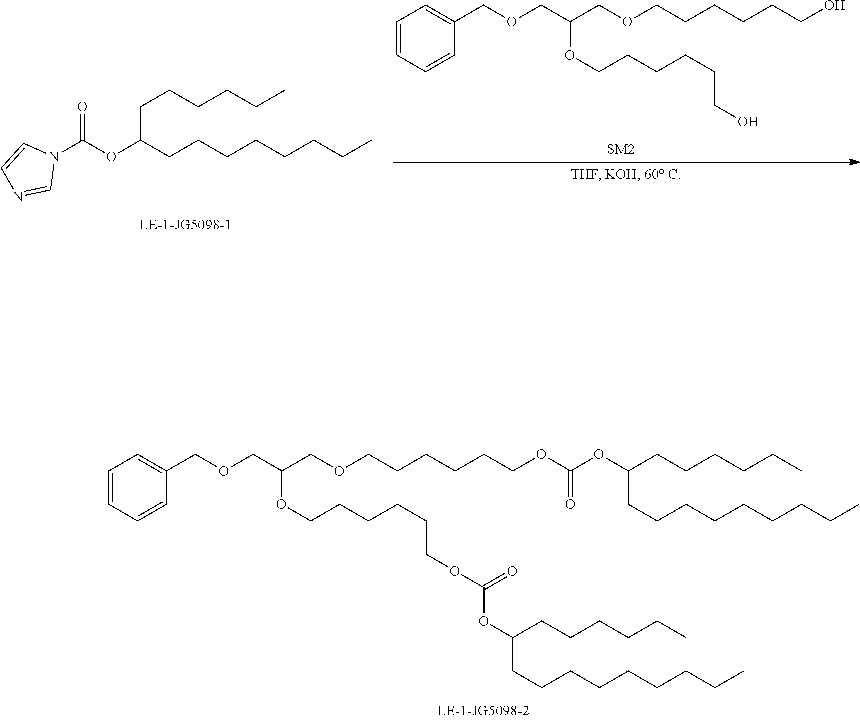 [SMILES: CCCCCCCCC(CCCCCC)OC(=O)n1ccnc1 ; OCCCCCCOCC(COCc1ccccc1)OCCCCCCO ; CCCCCCCCCC(CCCCCC)OC(=O)OCCCCCCOCC(COCc1ccccc1)OCCCCCCOC(=O)OC(CCCCCC)CCCCCCCCC]

A solution of 6-[3-benzyloxy-2-(6-hydroxyhexoxy) propoxy] hexan-1-ol (2.4 g, 6.27 mmol), 1-hexylnonyl imidazole-1-carboxylate (5.06 g, 15.7 mmol), hydroxypotassium (17.6 mg, 0.314 mmol) in THF (30 mL) was stirred at 60° C. for 16 h. Diluted with EA (100 mL), washed with brine (30 mL*3), dried with Na2SO4, filtered, purified by silica-gel (EA/PE=1/20), obtained 6-[3-benzyloxy-2-[6-(1-hexyldecoxycarbonyloxy) hexoxy] propoxy] hexyl 1-hexyldecyl carbonate (2.5 g, 2.72 mmol, 43.3% yield) as colorless oil.

1H NMR (400 MHZ, CDCl3) Õ 7.41-7.27 (m, 5H), 4.73-4.61 (m, 2H), 4.55 (s, 2H), 4.17-4.03 (m, 4H), 3.66-3.34 (m, 9H), 1.69-1.51 (m, 18H), 1.41-1.23 (m, 50H), 0.88 (t, J=6.8 Hz, 12H).

### Synthesis of the Intermediate LE-1-JG5098-3

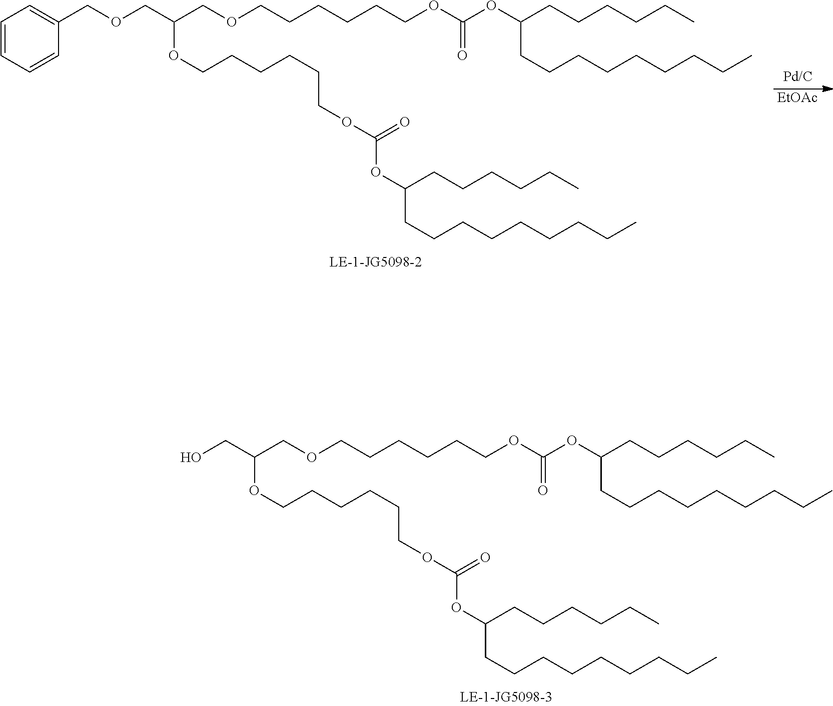 [SMILES: "EtOAc" ; CCCCCCCCCC(CCCCCC)OC(=O)OCCCCCCOCC(COCc1ccccc1)OCCCCCCOC(=O)OC(CCCCCC)CCCCCCCCC ; CCCCCCCCCC(CCCCCC)OC(=O)OCCCCCCOCC(CO)OCCCCCCOC(=O)OC(CCCCCC)CCCCCCCCC]

A solution of 6-[3-benzyloxy-2-[6-(1-hexyldecoxycarbonyloxy) hexoxy] propoxy] hexyl 1-hexyldecyl carbonate (2.5 g, 2.72 mmol), palladium (0.2 g, 0.43 mmol) in EA (50 mL) was stirred at room temperature under H₂ protection for 16 h. Filtered, concentrated to obtain 6-[2-[6-(1-hexyldecoxycarbonyloxy) hexoxy]-3-hydroxy-propoxy] hexyl 1-hexyldecyl carbonate (2.2 g, 2.65 mmol, 97.6% yield) as colorless oil.

1H NMR (400 MHZ, CDCl3) δ 4.75-4.61 (m, 2H), 4.12 (q, J=6.9 Hz, 4H), 3.79-3.35 (m, 9H), 1.80-1.49 (m, 18H), 1.42-1.21 (m, 50H), 0.88 (t, J=6.8 Hz, 12H).

### Synthesis of the Intermediate LE-1-JG5098-4

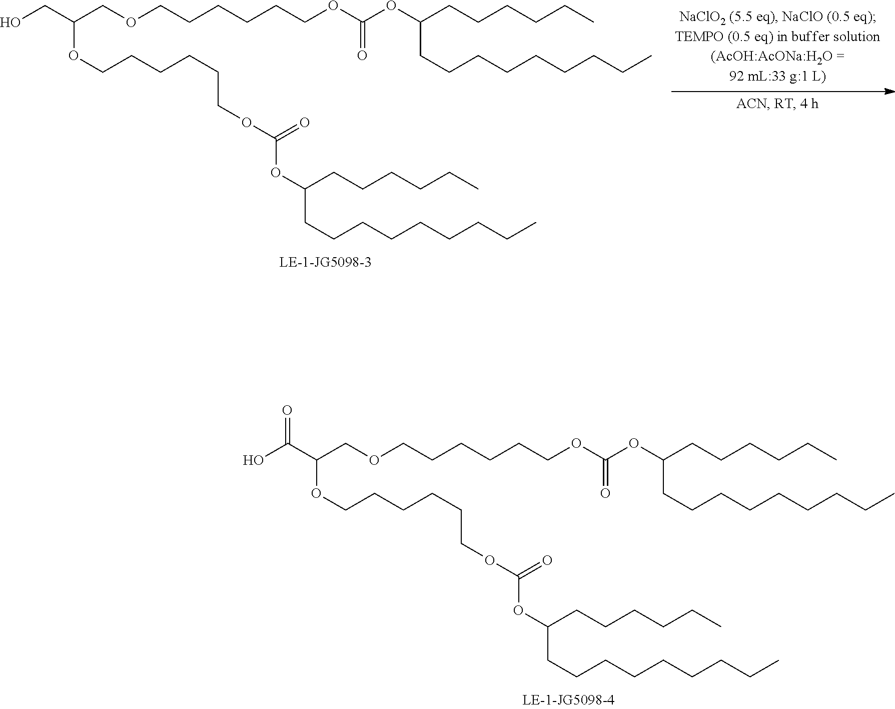 [SMILES: CCCCCCCCCC(CCCCCC)OC(=O)OCCCCCCOCC(CO)OCCCCCCOC(=O)OC(CCCCCC)CCCCCCCCC ; CCCCCCCCCC(CCCCCC)OC(=O)OCCCCCCOCC(OCCCCCCOC(=O)OC(CCCCCC)CCCCCCCCC)C(=O)O]

To a solution of 6-[2-[6-(1-hexyldecoxycarbonyloxy) hexoxy]-3-hydroxy-propoxy] hexyl 1-hexyldecyl carbonate (2.2 g, 2.65 mmol) in Acetonitrile (30 mL) and buffer solution (PH=4) (15 mL, AcOH: AcONa: water=92 mL: 33 g: 1000 mL) were added sodium chlorite (1.32 g, 14.6 mmol) and sodium hypochlorite (0.098 g, 1.33 mmol) and followed by TEMPO ((2,2,6,6-tetramethylpiperidin-1-yl)oxyl) (0.209 g, 1.33 mmol). The reaction became black and stirred at 20° C. for 4 hr. The reaction was quenched with 20 drops of methanol and was poured into water (40 mL) and extracted with ethyl acetate (60 ml×3). The organic layers were combined, washed with brine (60 mL), dried over anhydrous sodium sulfate, filtered and concentrated. The residue, combined with above batch, was purified through flash chromatography eluted with 5% to 10% methanol in dichloromethane to give 2, 3-bis [6-(1-hexyldecoxycarbonyloxy) hexoxy] propanoic acid (2.1 g, 93.9% yield) as light yellow oil.

1H NMR (400 MHZ, CDCl3) δ 4.78-4.60 (m, 2H), 4.12 (q, J=7.0 Hz, 4H), 4.04 (dd, J=4.9, 3.4 Hz, 1H), 3.82-3.39 (m, 6H), 1.74-1.48 (m, 16H), 1.46-1.14 (m, 51H), 0.88 (t, J=6.8 Hz, 12H).

### Synthesis of the Intermediate LE-1-JG5098-5

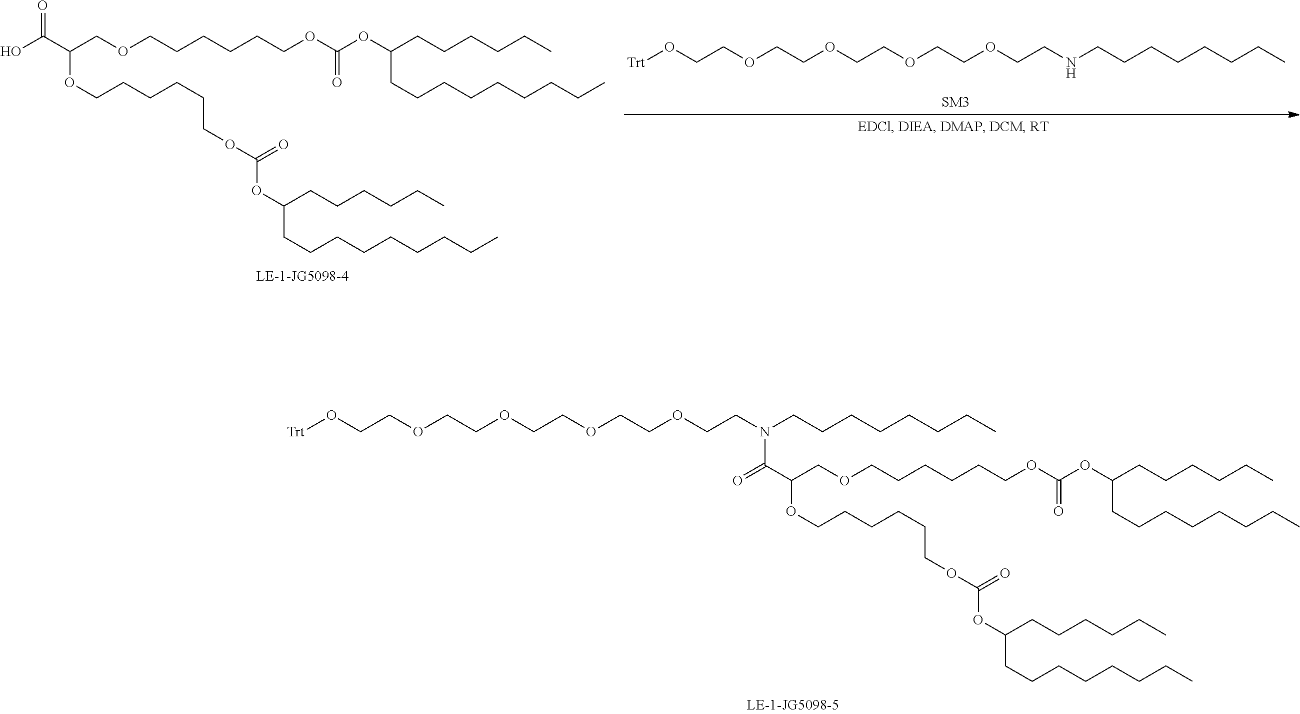

To a solution of 2,3-bis [6-(1-hexyldecoxycarbonyloxy) hexoxy] propanoic acid (1.5 g, 1.78 N-[2-[2-[2-[2-(2-trityloxyethoxy) ethoxy]ethoxy] ethoxy]ethyl] octan-1-amine (1.58 g, 2.67 mmol), DIPEA (0.92 g, 7.12 mmol), DMAP (0.217 g, 1.78 mmol) in dry dichloromethane (30 mL) was added EDCl (1.02 g, 5.34 mmol) at 0° C. The mixture was stirred at room temperature for 18 h. The reaction was washed with 1M HCl (aq) (10 mL) and brine (30 mL*2). The organic layer was dried over sodium sulfate, filtered and concentrated. The residue was purified by flash chromatography eluted with 25% EA in PE to give 6-[1-[6-(1-hexylnonoxycarbonyloxy) hexoxymethyl]-2-[octyl-[2-[2-[2-[2-(2-trityloxyethoxy) ethoxy]ethoxy] ethoxy]ethyl]amino]-2-oxo-ethoxy] hexyl 1-hexylnonyl carbonate (1.7 g, 1.22 mmol, 68.8% yield) as colorless oil.

1H NMR (400 MHZ, CDCl3) δ 7.53-7.38 (m, 6H), 7.32-7.18 (m, 9H), 4.76-4.58 (m, 2H), 4.46-4.25 (m, 1H), 4.17-4.01 (m, 4H), 3.73-3.19 (m, 28H), 1.62 (ddd, J=39.1, 27.8, 21.6 Hz, 20H), 1.39-1.23 (m, 56H), 0.88 (dd, J=8.3, 5.2 Hz, 15H).

### Synthesis of the Intermediate LE-1-JG5098-6

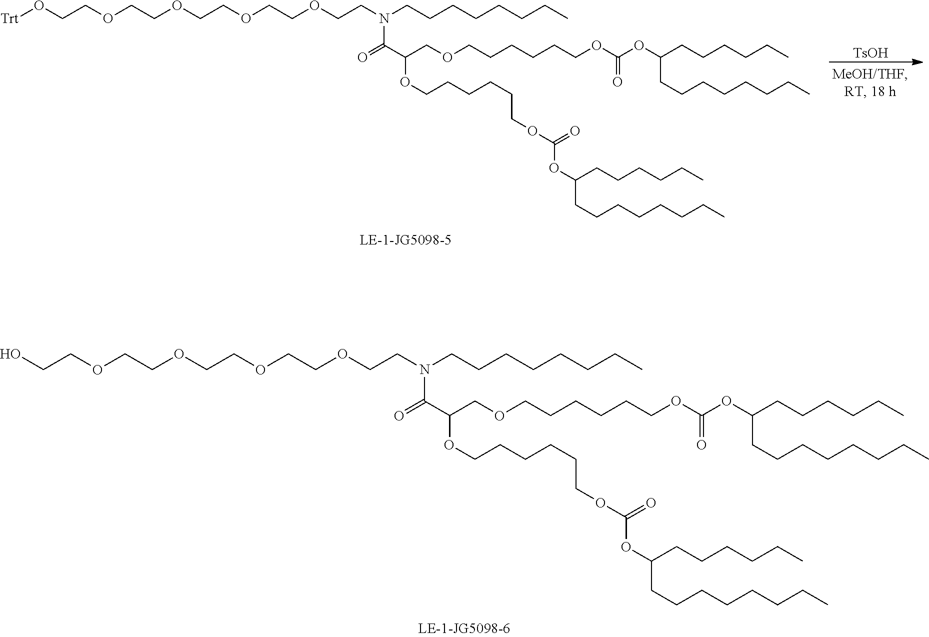

To a solution of 6-[1-[6-(1-hexylnonoxycarbonyloxy) hexoxymethyl]-2-[octyl-[2-[2-[2-[2-(2-trityloxyethoxy) ethoxy]ethoxy] ethoxy]ethyl]amino]-2-oxo-ethoxy] hexyl 1-hexylnonyl carbonate (1.7 g, 1.22 mmol), in MeOH (15 mL) and THF (15 mL) was added 4-methylbenzenesulfonic acid; hydrate (0.698 g, 3.67 mmol). The mixture was stirred at room temperature for 18 h. The reaction solution was diluted with EA (100 mL), washed with Na2CO₃ (aq) (30 mL) and brine (30 mL*2). The organic layer was dried over sodium sulfate, filtered and concentrated. The residue was purified by flash chromatography eluted with 5% MeOH in DCM to give 6-[2-[6-(1-hexylnonoxycarbonyloxy) hexoxy]-3-[2-[2-[2-[2-(2-hydroxyethoxy) ethoxy]ethoxy] ethoxy]ethyl-octyl-amino]-3-oxo-propoxy] hexyl 1-hexylnonyl carbonate (1.3 g, 1.13 mmol, 92.6% yield) as colorless oil.

1H NMR (400 MHZ, CDCl3) δ 4.74-4.60 (m, 2H), 4.46-4.28 (m, 1H), 4.15-4.04 (m, 4H), 3.74-3.38 (m, 28H), 1.70-1.50 (m, 18H), 1.41-1.23 (m, 58H), 0.88 (dd, J=9.0, 4.6 Hz, 15H).

### Synthesis of the Compound XXXVII

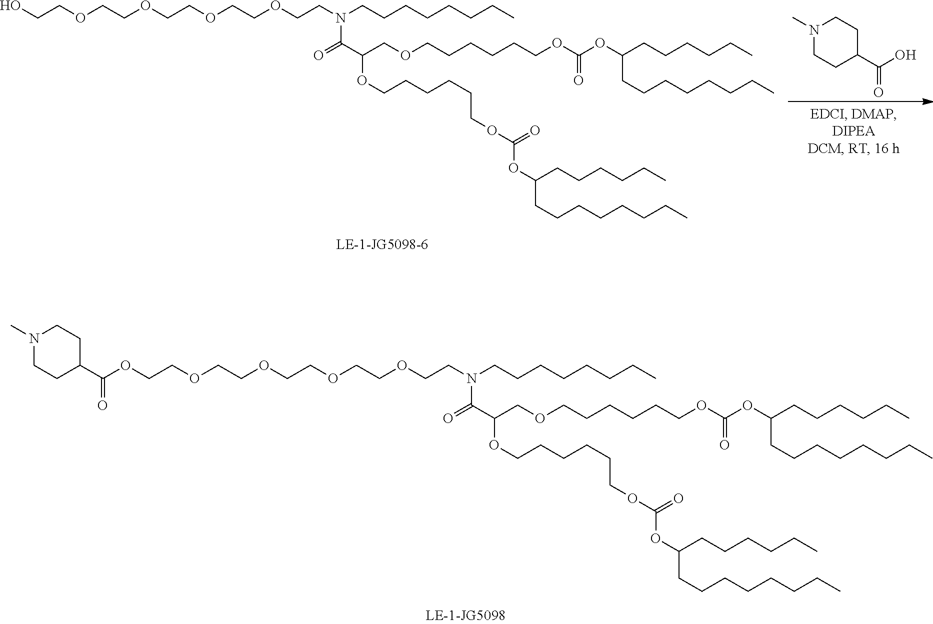

To a solution of 6-[2-[6-(1-hexylnonoxycarbonyloxy) hexoxy]-3-[2-[2-[2-[2-(2-hydroxyethoxy) ethoxy]ethoxy] ethoxy]ethyl-octyl-amino]-3-oxo-propoxy] hexyl 1-hexylnonyl carbonate (1.3 g, 1.13 mmol), 1-methylpiperidine-4-carboxylic acid (0.487 g, 3.4 mmol), DIPEA (0.586 g, 4.53 mmol), DMAP (0.138 g, 1.13 mmol) in DCM (30 mL) was added EDCl (0.649 g, 3.4 mmol) at 0° C. The mixture was stirred at room temperature for 18 h. The reaction was washed with Na2CO₃ (aq) (20 mL) and brine (30 mL*2). The organic layer was dried over sodium sulfate, filtered, and concentrated. The residue was purified by flash chromatography eluted with 5% MeOH in DCM to give 2-[2-[2-[2-[2-[2,3-bis [6-(1-hexylnonoxycarbonyloxy) hexoxy] propanoyl-octyl-amino]ethoxy]ethoxy] ethoxy]ethoxy]ethyl 1-methylpiperidine-4-carboxylate (1.05 g, 0.826 mmol, 72.8% yield) as colorless oil.

1H NMR (400 MHZ, CDCl3) δ 4.75-4.60 (m, 2H), 4.46-4.28 (m, 1H), 4.26-4.18 (m, 2H), 4.10 (td, J=6.6, 1.9 Hz, 4H), 3.72-3.57 (m, 19H), 3.52-3.25 (m, 7H), 2.82 (d, J=11.5 Hz, 2H), 2.35-2.21 (m, 4H), 1.97 (ddd, J=16.6, 14.7, 4.2 Hz, 4H), 1.83-1.75 (m, 2H), 1.69-1.51 (m, 18H), 1.32 (d, J=45.0 Hz, 58H), 0.88 (dd, J=9.1, 4.4 Hz, 15H).

### Example 30: Synthesis of 2-[2-[2-[2-[2,3-bis [6-(2-octyldecanoyloxy) hexoxy] propoxy] ethoxy]ethoxy] ethoxy]ethyl 1,4-dimethylpiperidine-4-carboxylate (compound XXXVIII)

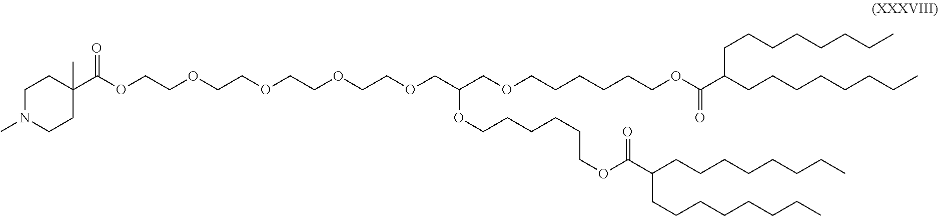 [SMILES: CCCCCCCCC(CCCCCCCC)C(=O)OCCCCCCOCC(COCCOCCOCCOCCOC(=O)C1(C)CCN(C)CC1)OCCCCCCOC(=O)C(CCCCCCCC)CCCCCCCC]

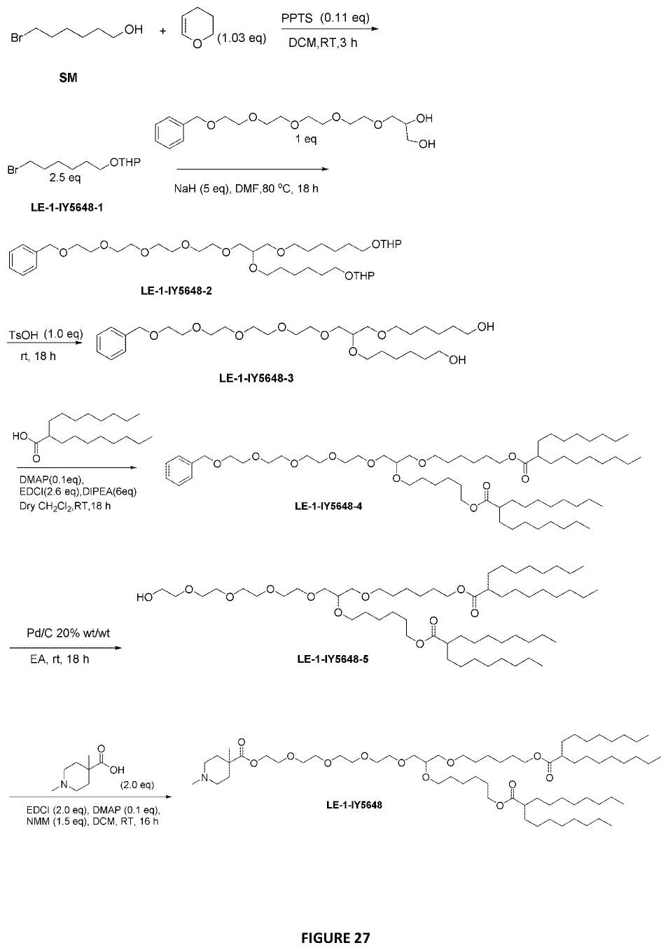

The compound XXXVIII is prepared according to the schema of synthesis of FIG. 27.

### Synthesis of the Intermediate LE-1-IY5648-4

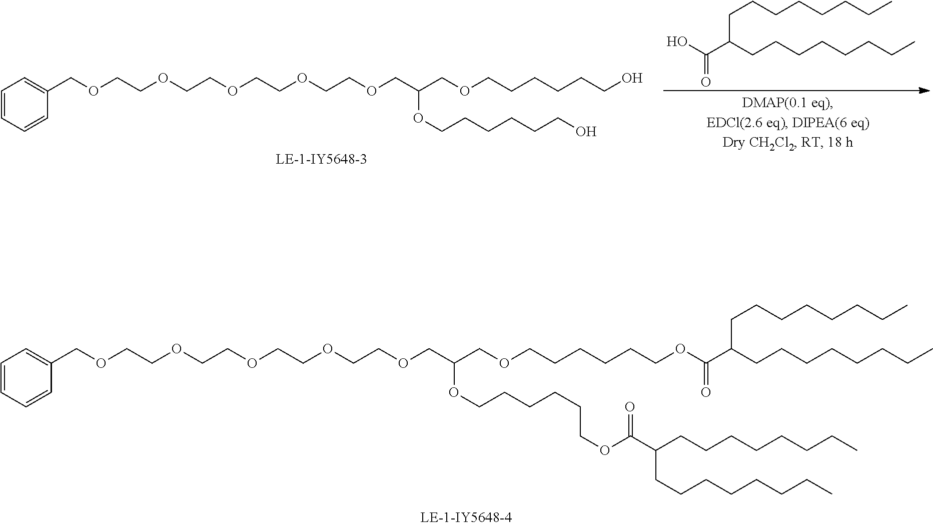 [SMILES: OCCCCCCOCC(COCCOCCOCCOCCOCc1ccccc1)OCCCCCCO ; CCCCCCCCC(CCCCCCCC)C(=O)O ; CCCCCCCCC(CCCCCCCC)C(=O)OCCCCCCOCC(COCCOCCOCCOCCOCc1ccccc1)OCCCCCCOC(=O)C(CCCCCCCC)CCCCCCCC]

To the solution of 16-((6-hydroxyhexyl)oxy)-1-phenyl-2,5,8, 11, 14, 18-hexaoxatetracosan-24-ol (1.1 g, 1.97 mmol) and 2-octyldecanoic acid (1.68 g, 5.91 mmol) in dry dichloromethane (10 mL) were added DIPEA (1.53 g, 11.8 mmol), DMAP (0.096 g, 0.78 mmol) under ice bath, then EDCl (1.13 g, 5.91 mmol) added. The mixture was stirred at room temperature for 18 h. The reaction was washed with 0.1 M HCl and NaHCO₃ and brine. The organic layer was dried over sodium sulfate, filtered, and concentrated. The residue was purified by flash chromatography eluted with 0% to 5% CH₃OH in DCM to give6-[3-[2-[2-[2-(2-benzyloxyethoxy) ethoxy]ethoxy] ethoxy]-2-[6-(2-octyldecanoyloxy) hexoxy] propoxy] hexyl 2-octyldecanoate (1.3 g, 1.19 mmol, 60% yield) as a colorless oil.

1H NMR (400 MHZ, CDCl3) δ 7.38-7.30 (m, 4H), 4.57 (s, 2H), 4.05 (td, J=6.6, 1.5 Hz, 4H), 3.80-3.57 (m, 16H), 3.58-3.28 (m, 9H), 2.38-2.22 (m, 2H), 1.66-1.50 (m, 12H), 1.48-1.33 (m, 12H), 1.32-1.11 (m, 48H), 0.87 (t, J=6.8 Hz, 12H).

### Synthesis of the Intermediate LE-1-IY5648-5

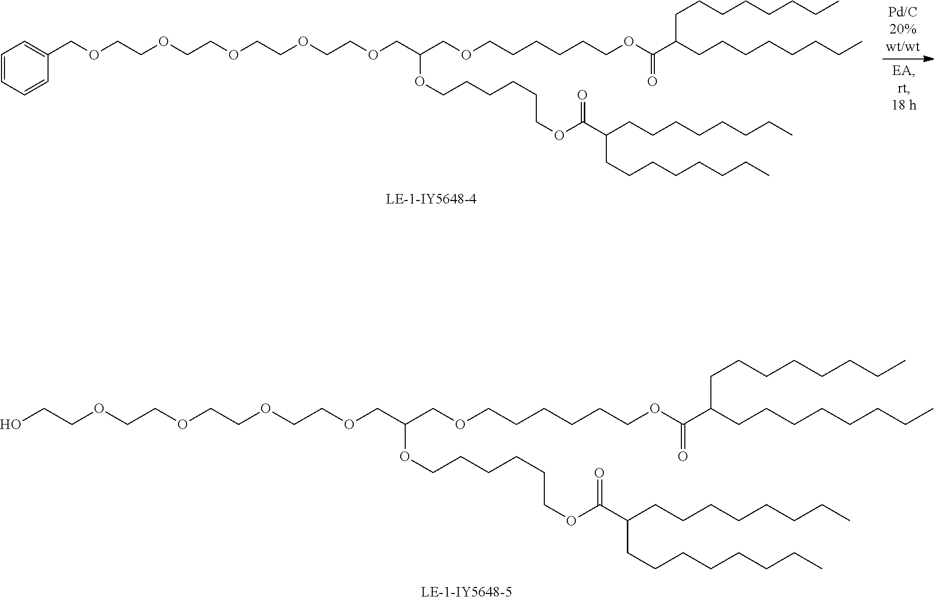 [SMILES: CCCCCCCCC(CCCCCCCC)C(=O)OCCCCCCOCC(COCCOCCOCCOCCOCc1ccccc1)OCCCCCCOC(=O)C(CCCCCCCC)CCCCCCCC ; CCCCCCCCC(CCCCCCCC)C(=O)OCCCCCCOCC(COCCOCCOCCOCCO)OCCCCCCOC(=O)C(CCCCCCCC)CCCCCCCC]

A solution of 6-[3-[2-[2-[2-(2-benzyloxyethoxy) ethoxy]ethoxy] ethoxy]-2-[6-(2-octyldecanoyloxy) hexoxy] propoxy] hexyl 2-octyldecanoate (1.3 g, 1.19 mmol) in EtOAc (50 mL) was purged for 10 minutes with N2 followed by addition of Pd/C (260 mg) and the reaction continued purging with N2. The reaction was next evacuated under vacuum and backfilled with H₂ for three times. The reaction was stirred overnight at room temperature under H₂ ballon. TLC (5% CH₃OH in DCM) shows that the reaction was finished. The reaction was filtered through celite and the celite was washed with EtOAc. The organic phase was concentrated under vacuum to give 6-[3-[2-[2-[2-(2-hydroxyethoxy) ethoxy]ethoxy] ethoxy]-2-[6-(2-octyldecanoyloxy) hexoxy] propoxy] hexyl 2-octyldecanoate (1.1 g, 1.1 mmol, 92.2% yield) as colorless oil.

1H NMR (400 MHZ, CDCl3) δ 4.15-3.99 (m, 4H), 3.75-3.59 (m, 16H), 3.59-3.36 (m, 8H), 2.35-2.19 (m, 2H), 1.68-1.50 (m, 12H), 1.45-1.31 (m, 12H), 1.27 (d, J=16.6 Hz, 48H), 0.88 (t, J=6.8 Hz, 12H).

### Synthesis of the Compound XXXVIII

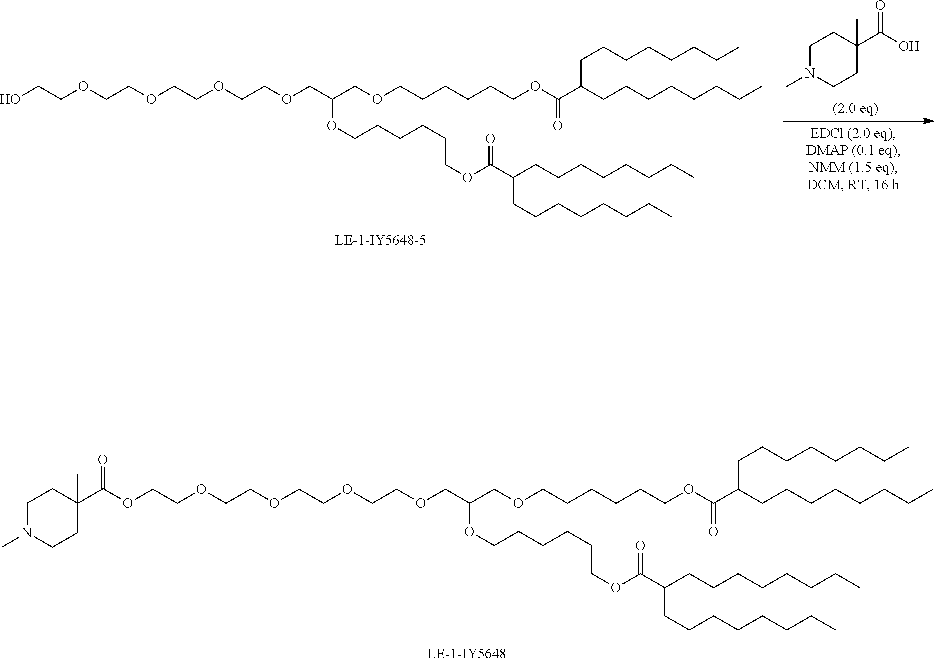

DIPEA (0.9 g, 6.99 mmol) was added to the solution of 1,4-dimethylpiperidine-4-carboxylic acid (0.33 g, 2.1 mmol) in dry dichloromethane (25 mL), stirred for 10 min until the solution was clear. 6-[3-[2-[2-[2-(2-hydroxyethoxy) ethoxy]ethoxy] ethoxy]-2-[6-(2-octyldecanoyloxy) hexoxy] propoxy] hexyl 2-octyldecanoate (0.7 g, 0.69 mmol), DMAP (0.042 g, 0.34 mmol) and EDCl (0.4 g, 2.1 mmol) were added under ice bath. The mixture was stirred at room temperature for 18 h. The reaction was washed with Na2CO₃ (30 mL) and brine. The organic layer was dried over sodium sulfate, filtered and concentrated. The residue was purified by flash chromatography eluted with 0% to 5% CH₃OH in DCM to give 2-[2-[2-[2-[2,3-bis [6-(2-octyldecanoyloxy) hexoxy] propoxy] ethoxy]ethoxy] ethoxy]ethyl 1,4-dimethylpiperidine-4-carboxylate (0.420 g, 0.36 mmol, 52.7% yield) as a colorless oil.

1H NMR (400 MHZ, CDCl3) δ 4.37-4.24 (m, 2H), 4.13-3.98 (m, 4H), 3.73-3.39 (m, 23H), 3.23-3.03 (m, 2H), 2.75-2.48 (m, 5H), 2.29 (ddd, J=21.4, 13.2, 9.0 Hz, 4H), 2.04 (d, J=11.7 Hz, 2H), 1.72-1.13 (m, 75H), 0.88 (t, J=6.8 Hz, 12H).

### Example 31: Synthesis of 2-[2-[2-[2-[2,3-bis [8-oxo-8-(3-pentyloctoxy) octoxy] propoxy] ethoxy]ethoxy] ethoxy]ethyl 1-methylpiperidine-4-carboxylate (compound XXXIX)

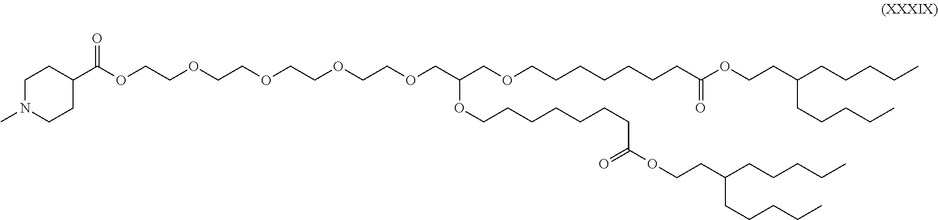 [SMILES: CCCCCC(CCCCC)CCOC(=O)CCCCCCCOCC(COCCOCCOCCOCCOC(=O)C1CCN(C)CC1)OCCCCCCCC(=O)OCCC(CCCCC)CCCCC]

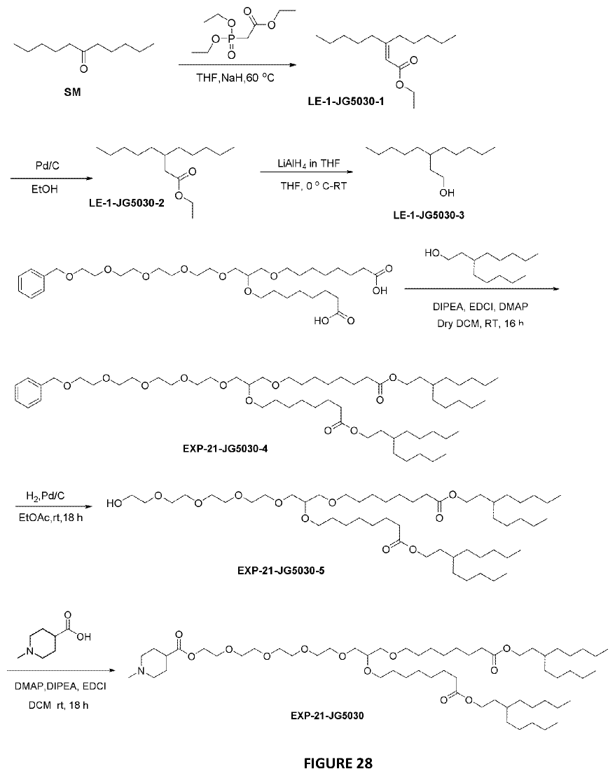

The compound XXXIX is prepared according to the schema of synthesis of FIG. 28.

### Synthesis of the Intermediate LE-1-JG5030-1

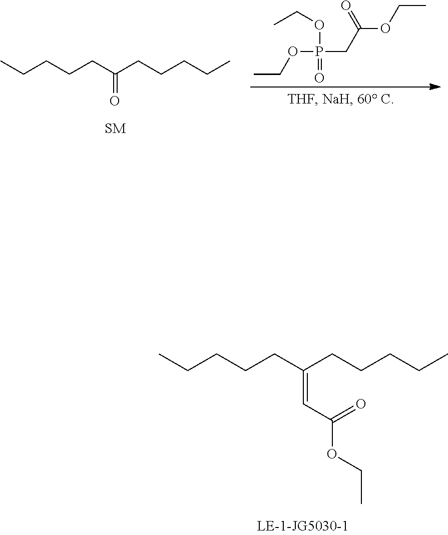

To a solution of ethyl 2-(diethoxyphosphoryl) acetate (138 g, 658 mmol) in 2000 mL THF at 0° C. was added NaH (26.3 g, 658 mmol) and the mixture was stirred at 0° C. for 1 hour then undecan-6-one (14 g, 82.2 mmol) was added and the resulting reaction mixture was stirred for 16 hours at 60° C. under N2. The reaction mixture was poured into water (2000 ml) and separated by EA (2000 ml *2). Organic layer was washed with H₂O and dried over Na2SO4 concentrated to give a viscous residue. The crude product was purified by silica gel chromatography eluted with PE: EA=10:1 to give product (19 g, 96.1%) as colorless oil.

### Synthesis of the Intermediate LE-1-JG5030-2

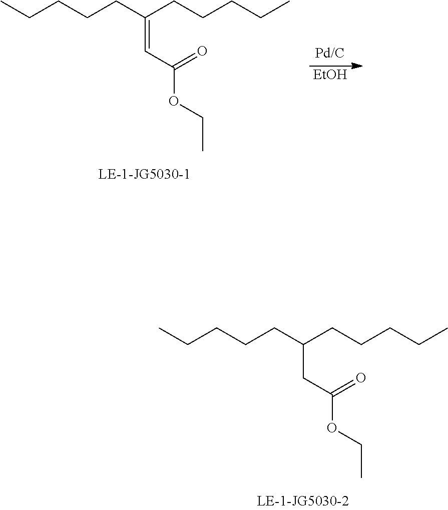

To a mixture of ethyl 3-pentyloct-2-enoate (20 g, crude) and Pd/C (4.43 g, 10%) was added in EtOH (100 ml). The mixture reaction was stirred at room temperature for 16 h under H₂. The suspension was filtered through a pad of Celite was washed with EtOH (200 ml *2). The mixture was concentrated under vacuum to get oil product (19 g, 94.2%).

1H NMR (400 MHZ, CDCl3) δ 4.12 (d, J=7.1 Hz, 2H), 2.22 (d, J=6.9 Hz, 2H), 1.84 (s, 1H), 1.36-1.14 (m, 23H), 0.88 (t, J=6.9 Hz, 7H).

### Synthesis of the Intermediate LE-1-JG5030-3

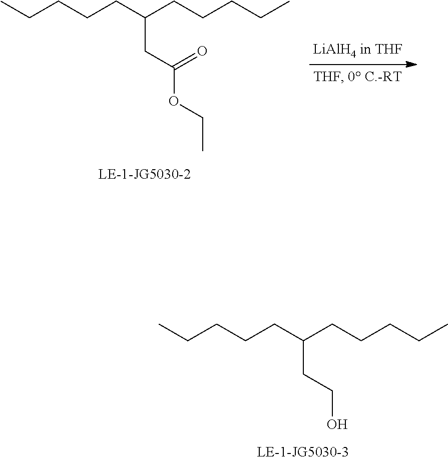

To a mixture of LiAlH₄ (1.88 g) in THF (50 ml) was dropped wise in ethyl 3-pentyloctanoate (12 g, 49.5 mmol) in THF (50 ml). The mixture reaction was stirred at 0° C. for 2 h under N2. The reaction mixture was quenched by addition of 100 mL of H₂O, followed by 100 mL of 15% aqueous NaOH. After being stirred at room temperature for 1 hour, the solid was removed by filtration. The filtrate was concentrated to dryness to give crude product, which was purified by silica gel chromatography eluted with PE: EA=10:1 to give product (9.5 g, 95.8%) as colorless oil.

1H NMR (400 MHZ, CDCl3) δ 3.66 (t, J=7.1 Hz, 2H), 1.52 (t, J=7.0 Hz, 2H), 1.42 (d, J=4.5 Hz, 1H), 1.35-1.17 (m, 17H), 0.89 (t, J=7.0 Hz, 6H).

### Synthesis of the Intermediate LE-1-JG5030-4

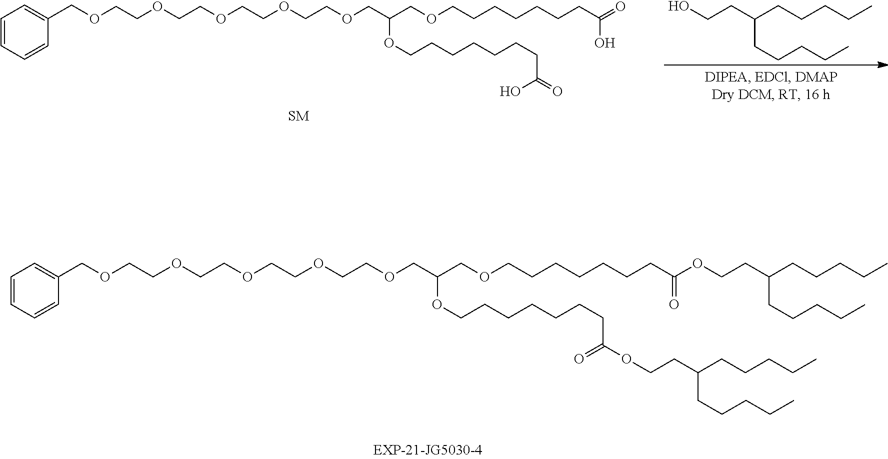 [SMILES: O=C(O)CCCCCCCOCC(COCCOCCOCCOCCOCc1ccccc1)OCCCCCCCC(=O)O ; CCCCCC(CCO)CCCCC ; CCCCCC(CCCCC)CCOC(=O)CCCCCCCOCC(COCCOCCOCCOCCOCc1ccccc1)OCCCCCCCC(=O)OCCC(CCCCC)CCCCC]

A mixture of 8-[3-[2-[2-[2-(2-benzyloxyethoxy) ethoxy]ethoxy] ethoxy]-2-(7-carboxyheptoxy) propoxy] octanoic acid (1.0 g, 1.56 mmol), 3-pentyloctan-1-ol (1.25 g, 6.22 mmol), N-(3-DIMETHYLAMINOPROPYL)-N′-ETHYLCARBODIIMIDE HYDROCHLORIDE (494 mg, 4.04 mmol), N,N-dimethylpyridin-4-amine (76 mg, 0.622 mmol) and N-ethyl-N-isopropyl-propan-2-amine (1.21 g, 9.33 mmol) in DCM (20 mL) was stirred at room temperature 16 hrs. The solvent was concentrated, the residue was purified by column chromatography with methanol in DCM (0-10%) to give 3-pentyloctyl 8-[3-[2-[2-[2-(2-benzyloxyethoxy) ethoxy]ethoxy] ethoxy]-2-[8-oxo-8-(3-pentyloctoxy) octoxy] propoxy] octanoate (1.2 g, purity: 95%, yield: 72.7%) as pale yellow oil.

1H NMR (400 MHz, CDCl3) δ 4.60 (s, 2H), 4.39-4.32 (m, 2H), 4.10-3.94 (m, 8H), 3.84 (s, 2H), 3.70 (s, 4H), 3.62 (s, 12H), 3.48 (s, 2H), 3.35 (d, J=10.4 Hz, 7H), 3.15 (d, J=12.8 Hz, 4H), 2.35-2.26 (m, 2H), 1.76-1.51 (m, 12H), 1.49-1.33 (m, 12H), 1.25 (s, 40H), 0.87 (t, J=6.4 Hz, 12H).

### Synthesis of the Intermediate LE-1-JG5030-5

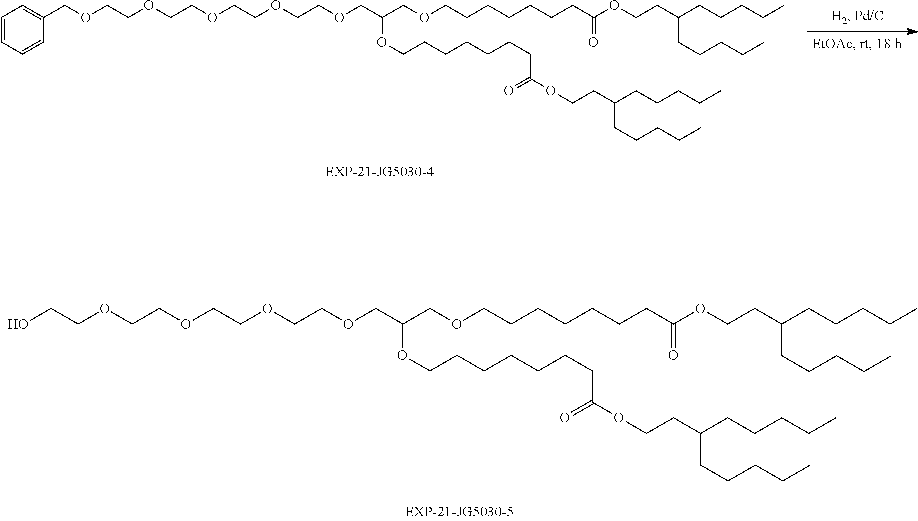 [SMILES: CCCCCC(CCCCC)CCOC(=O)CCCCCCCOCC(COCCOCCOCCOCCOCc1ccccc1)OCCCCCCCC(=O)OCCC(CCCCC)CCCCC ; CCCCCC(CCCCC)CCOC(=O)CCCCCCCOCC(COCCOCCOCCOCCO)OCCCCCCCC(=O)OCCC(CCCCC)CCCCC]

A mixture of 3-pentyloctyl 8-[3-[2-[2-[2-(2-benzyloxyethoxy) ethoxy]ethoxy] ethoxy]-2-[8-oxo-8-(3-pentyloctoxy) octoxy] propoxy] octanoate (1.2 g, 1.19 mmol) in EtOAc (10 mL) was added palladium (300 mg) and through gas H₂, the mixture was stirred at room temperature for 18 hours. Filtered and concentrated to give 3-pentyloctyl 8-[3-[2-[2-[2-(2-hydroxyethoxy) ethoxy]ethoxy] ethoxy]-2-[8-oxo-8-(3-pentyloctoxy) octoxy] propoxy] octanoate (650 mg, purity: 95%, yield: 56.5%) as an oil.

1H NMR (400 MHZ, CDCl3) δ 4.01 (t, J=7.6 Hz, 4H), 3.66-3.33 (m, 25H), 2.21 (t, J=7.6 Hz, 4H), 1.52-1.47 (m, 12H), 1.33-1.18 (m, 49H), 0.81 (t, J=6.8 Hz, 12H).

### Synthesis of the Compound XXXIX

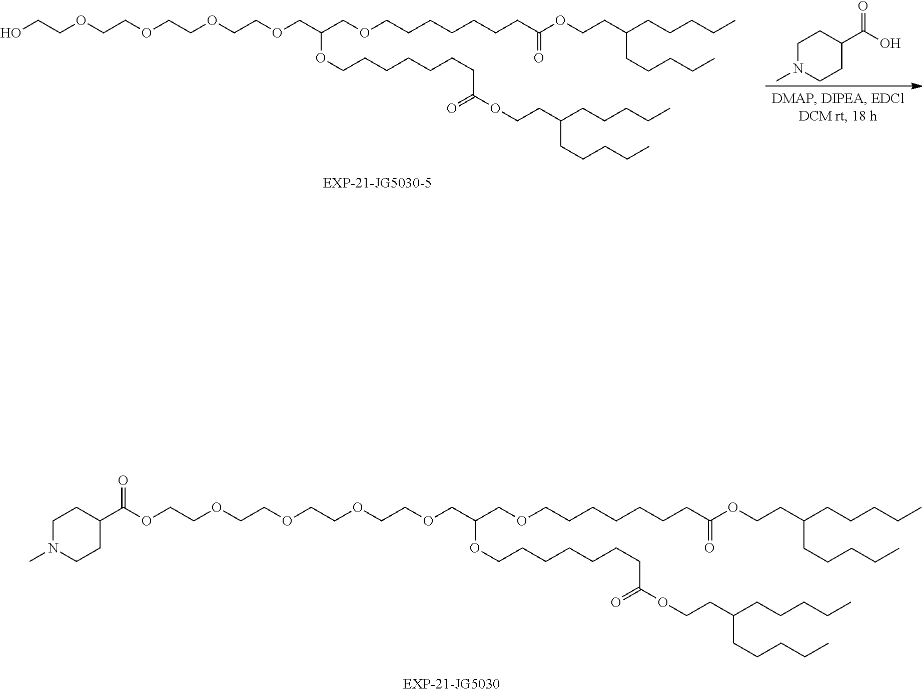

A mixture of 3-pentyloctyl8-[3-[2-[2-[2-(2-hydroxyethoxy) ethoxy]ethoxy] ethoxy]-2-[8-oxo-8-(3-pentyloctoxy) octoxy] propoxy] octanoate (650 mg, 0.709 mmol), 1-methylpiperidine-4-carboxylic acid (304 mg, 2.13 mmol), N-(3-dimethylaminopropyl)-N′-ethylcarbodiimidehydrochloride (353 mg, 1.84 mmol), N,N-dimethylpyridin-4-amine (17.3 mg, 0.142 mmol) and N-ethyl-N-isopropyl-propan-2-amine (0.74 mL, 4.25 mmol) in 20 mL DCM was stirred at room temperature 16 hrs. The solvent was evaporated (50 mL DCM*3). Then purified by column chromatography with DCM in CH₃OH (0-10%) to give 2-[2-[2-[2-[2,3-bis [8-oxo-8-(3-pentyloctoxy) octoxy] propoxy] ethoxy]ethoxy] ethoxy]ethyl 1-methylpiperidine-4-carboxylate (600 mg, quant.) as pale yellow oil.

1H NMR (400 MHZ, CDCl3) Õ 4.60 (s, 2H), 4.39-4.32 (m, 2H), 4.10-3.94 (m, 8H), 3.84 (s, 2H), 3.70 (s, 4H), 3.62 (s, 12H), 3.48 (s, 2H), 3.35 (d, J=10.4 Hz, 7H), 3.15 (d, J=12.8 Hz, 4H), 2.35-2.26 (m, 2H), 1.76-1.51 (m, 12H), 1.49-1.33 (m, 12H), 1.25 (s, 40H), 0.87 (t, J=6.4 Hz, 12H).

### Example 32: Synthesis of Synthesis of -bis(2-butyloctyl) 10-[3-[2-[(1-methylpiperidine-4-carbonyl) amino]ethyldisulfanyl] propanoyl-nonyl-amino] nonadecanedioate (compound XLVII)

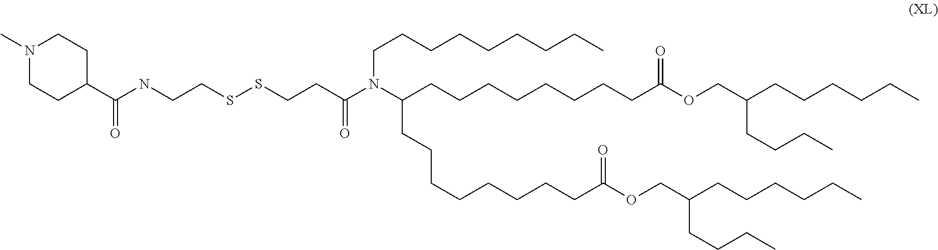 [SMILES: CCCCCCCCCN(C(=O)CCSSCCNC(=O)C1CCN(C)CC1)C(CCCCCCCCC(=O)OCC(CCCC)CCCCCC)CCCCCCCCC(=O)OCC(CCCC)CCCCCC]

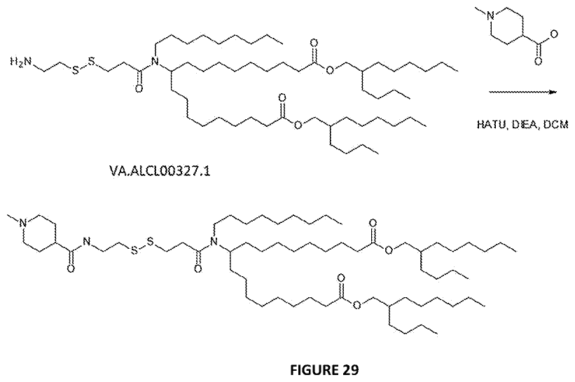

The compound XLVII is prepared according to the schema of synthesis of FIG. 29.

### For the Synthesis of the Intermediate VA.ALCL00327.1, Refer to Example 29. FIG. 23B

### Synthesis of the Compound XLVII

In a 10 mL microwave tube on an orbital shaker, the intermediate bis(2-butyloctyl) 10-[3-(2-aminoethyldisulfanyl) propanoyl-nonyl-amino] nonadecanedioate; hydrochloride, and 4 mL DCM are loaded. HATU (0.2505 mmol, 95.2481 mg) and N,N-Diisopropylethylamine (0.5367 mmol, 0.096 mL, 69.3648 mg) are added, then add the 1-methylpiperidine-4-carboxylic acid (35,8675 mg, 0,2505 mmol). Stirring at room temperature overnight. Concentrate the MR to dryness then purify by flash chromatography: 15 g merck silica cartridge, Eluent: DCM/MeOH, Gradient from 100/0 to 90/10 in 35 min there by obtaining 118 mg, 59% bis(2-butyloctyl) 10-[3-[2-[(1-methyl piperidine-4 carbonyl) amino]ethyldisulfanyl] propanoyl-nonyl-amino] nonadecanedioate as colorless oil.

1H NMR (400 MHZ, DMSO-d6, 80° C.) δ ppm 0.82-0.91 (m, 15H), 1.11-1.73 (m, 77H), 1.85-2.12 (m, 4H), 2.19 (br s, 3H), 2.25 (t, J=7.3 Hz, 4H), 2.41-3.85 (m, 3H), 2.64-2.70 (m, 2H), 2.77 (br t, J-6.8 Hz, 2H), 2.74-2.85 (m, 2H), 2.88-2.96 (m, 2 H), 2.98-3.19 (m, 2H), 3.27-3.40 (m, 2H), 3.92 (d, J=5.8 Hz, 4H), 7.55-7.73 (m, 1H).

### Example 33: Synthesis of 2-[2-[2-[2-[2,3-bis [8-(1-butylheptoxy)-8-oxo-octoxy] propoxy] ethoxy]ethoxy] ethoxy]ethyl 1,4-dimethylpiperidine-4-carboxylate (compound XLI)

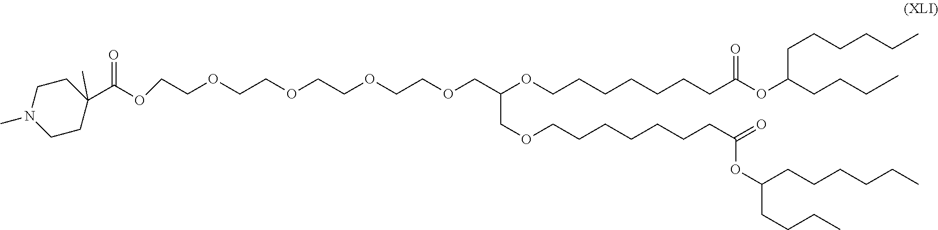 [SMILES: CCCCCCC(CCCC)OC(=O)CCCCCCCOCC(COCCOCCOCCOCCOC(=O)C1(C)CCN(C)CC1)OCCCCCCCC(=O)OC(CCCC)CCCCCC]

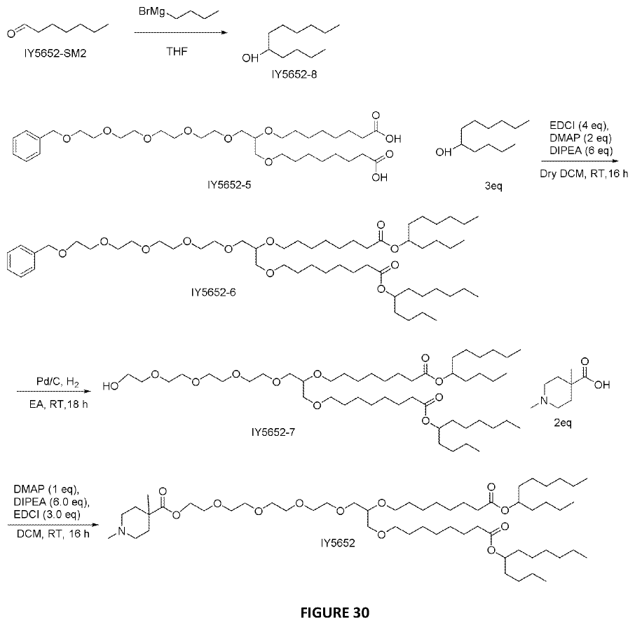

The compound XLI is prepared according to the schema of synthesis of FIG. 30.

### Synthesis of the Intermediate LE-1-IY5652-8

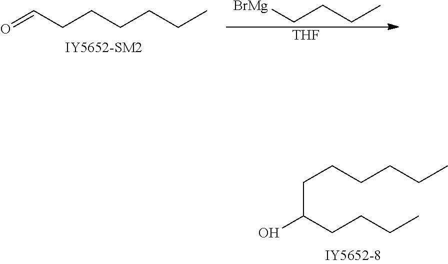

To a mixture of heptanal (5 g, 43.8 mmol) and in tetrahydrofuran (50 mL) was added bromo (butyl) magnesium (8.48 g, 52.5 mmol) slowly at 0° C. The mixture was stirred overnight at room temperature. EtOAc (100 mL) was added to the solution, and the mixture was washed with HCl (1M, 200 mL). The mixture was shaken, the layers were separated, and the organic layer was collected. The organic layer was washed with water (200 mL) and brine (200 mL) and dried over Na2SO4. Solvent was removed and the residue was purified by flash chromatography eluted with 0% to 10% ethyl acetate in petroleum ether to give undecan-5-ol (1.1 g, 14.6%) as a colorless oil.

1H NMR (400 MHZ, CDCl3) δ 3.71-3.47 (m, 1H), 1.52-1.15 (m, 16H), 0.90 (dt, J=9.3, 6.9 Hz, 6H).

### Synthesis of the Intermediate LE-1-IY5652-6

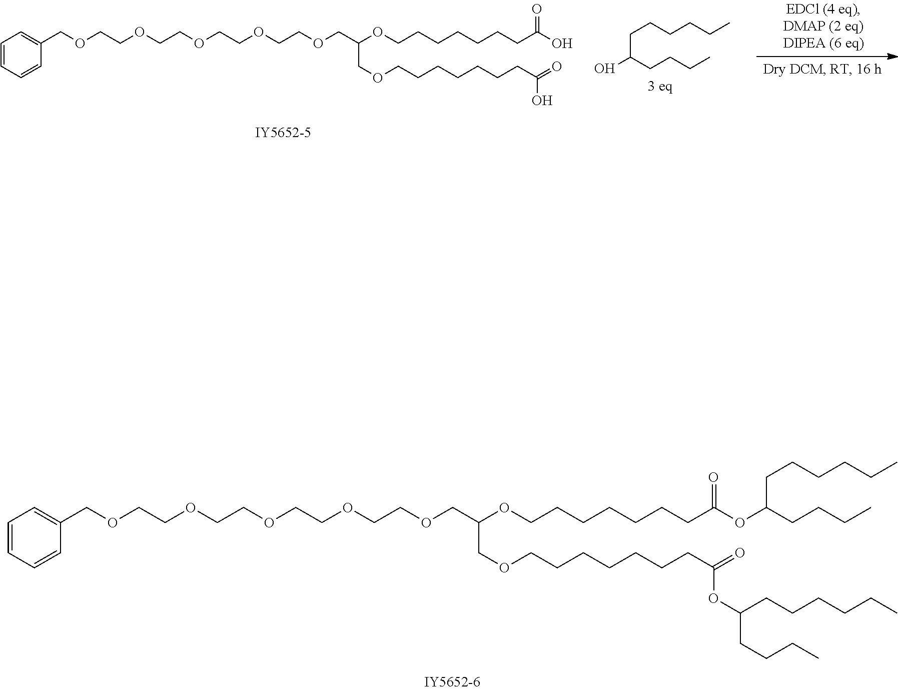 [SMILES: O=C(O)CCCCCCCOCC(COCCOCCOCCOCCOCc1ccccc1)OCCCCCCCC(=O)O ; CCCCCCC(O)CCCC ; CCCCCCC(CCCC)OC(=O)CCCCCCCOCC(COCCOCCOCCOCCOCc1ccccc1)OCCCCCCCC(=O)OC(CCCC)CCCCCC]

To a solution of 8-[3-[2-[2-[2-(2-benzyloxyethoxy) ethoxy]ethoxy] ethoxy]-2-(7-carboxyheptoxy) propoxy] octanoic acid (1.1 g, 1.7 mmol) and undecan-5-ol (0.88 g, 5.13 mmol) in dry dichloromethane (20 mL) were added DIPEA (1.11 g, 8.56 mmol), DMAP (0.209 g, 1.71 mmol) and followed by EDCl (1.64 g, 8.56 mmol). The mixture was stirred at room temperature for 18 h. The reaction was diluted with dichloromethane and washed with brine. The organic layer was dried over sodium sulfate, filtered and concentrated. The residue was purified by flash chromatography eluted with 0% to 10% MeOH in DCM to give 1-butylheptyl 8-[3-[2-[2-[2-(2-benzyloxyethoxy) ethoxy]ethoxy] ethoxy]-2-[8-(1-butylheptoxy)-8-oxo-octoxy] propoxy] octanoate (0.76 g, 46.7% yield) as light yellow oil.

1H NMR (400 MHZ, CDCl3) δ 7.31 (dd, J=23.6, 8.0 Hz, 4H), 4.97-4.73 (m, 2H), 4.12 (q, J=7.1 Hz, 4H), 3.73-3.28 (m, 25H), 2.27 (t, J=7.4 Hz, 4H), 1.61-1.42 (m, 31H), 1.27 (dd, J=17.5, 10.3 Hz, 42H), 0.92-0.81 (m, 12H).

### Synthesis of the Intermediate LE-1-IY5652-7

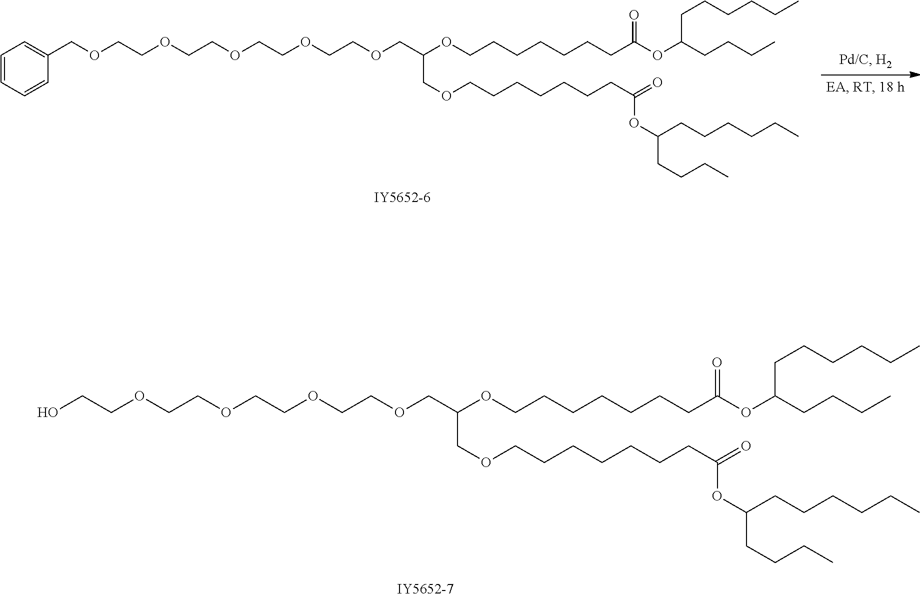 [SMILES: CCCCCCC(CCCC)OC(=O)CCCCCCCOCC(COCCOCCOCCOCCOCc1ccccc1)OCCCCCCCC(=O)OC(CCCC)CCCCCC ; CCCCCCC(CCCC)OC(=O)CCCCCCCOCC(COCCOCCOCCOCCO)OCCCCCCCC(=O)OC(CCCC)CCCCCC]

A mixture of 1-butylheptyl 8-[3-[2-[2-[2-(2-benzyloxyethoxy) ethoxy]ethoxy] ethoxy]-2-[8-(1-butylheptoxy)-8-oxo-octoxy] propoxy] octanoate (0.76 g, 0.8 mmol) in EtOAc (15 mL) was added palladium (100 mg) and through gas H₂, the mixture was stirred at room temperature for 16 hours. Filtered and concentrated to give 1-butylheptyl 8-[2-[8-(1-butylheptoxy)-8-oxo-octoxy]-3-[2-[2-[2-(2-hydroxyethoxy) ethoxy]ethoxy] ethoxy] propoxy] octanoate (0.46 g, yield: 66.9%) as a colorless?oil.

### Synthesis of the Compound XLI

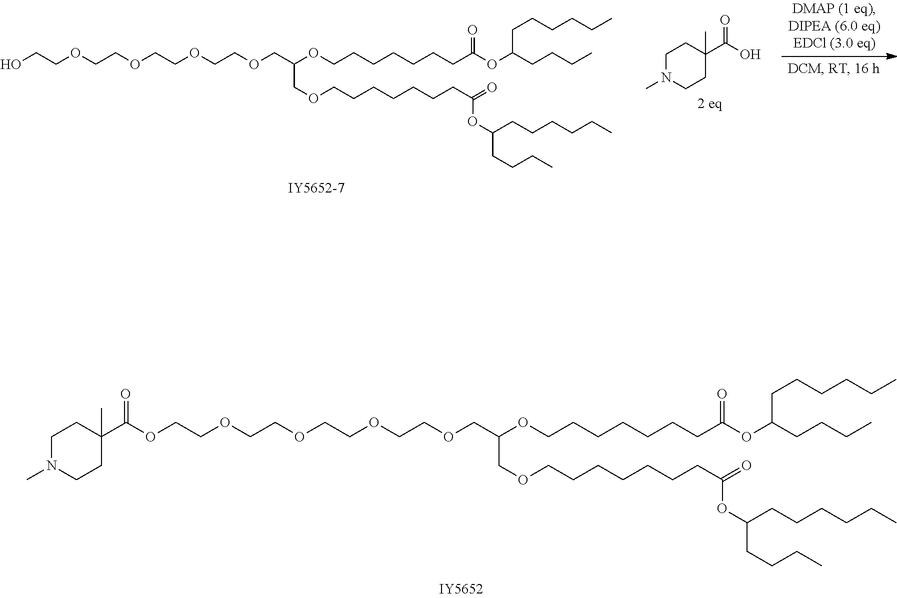

To a solution of 1-butylheptyl 8-[2-[8-(1-butylheptoxy)-8-oxo-octoxy]-3-[2-[2-[2-(2-hydroxyethoxy) ethoxy]ethoxy] ethoxy] propoxy] octanoate (0.46 g, 0.53 mmol) and 1,4-dimethylpiperidine-4-carboxylic acid (0.16 g, 1.07 mmol) in dry dichloromethane (10 mL) were added DIPEA (0.345g, 2.67 mmol), DMAP (0.065 g, 0.53 mmol) and followed by EDCl (0.41 g, 2.14 mmol). The mixture was stirred at room temperature for 18 h. The reaction was diluted with dichloromethane (50 mL) and washed with brine. The organic layer was dried over sodium sulfate, filtered, and concentrated. The residue was purified by flash chromatography eluted with 0% to 10% MeOH in DCM to give2-[2-[2-[2-[2,3-bis [8-(1-butylheptoxy)-8-oxo-octoxy] propoxy] ethoxy]ethoxy] ethoxy]ethyl 1,4-dimethylpiperidine-4-carboxylate (0.265 g, 49.6% yield) as light yellow oil.

1H NMR (400 MHZ, CDCl3) δ 4.94-4.78 (m, 2H), 4.36-4.19 (m, 2H), 3.79-3.29 (m, 21H), 2.91-2.57 (m, 1H), 2.44-2.08 (m, 9H), 1.55 (dd, J=28.9, 15.7 Hz, 20H), 1.27 (dd, J=25.7, 12.7 Hz, 40H), 0.95-0.77 (m, 12H).

### Example 34: Synthesis of 2-[2-[2-[2-[2,3-bis [8-(1-ethylpentoxy)-8-oxo-octoxy] propoxy] ethoxy]ethoxy] ethoxy]ethyl 1,4-dimethylpiperidine-4-carboxylate (compound XLII)

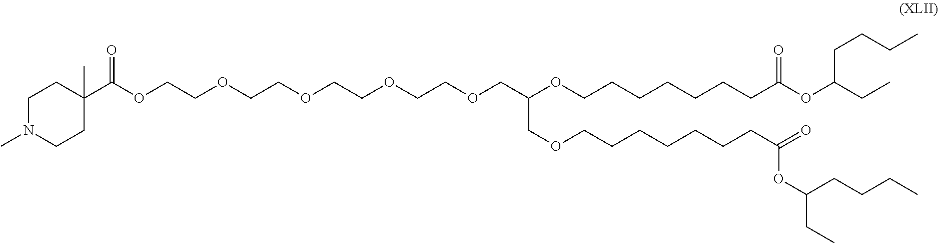 [SMILES: CCCCC(CC)OC(=O)CCCCCCCOCC(COCCOCCOCCOCCOC(=O)C1(C)CCN(C)CC1)OCCCCCCCC(=O)OC(CC)CCCC]

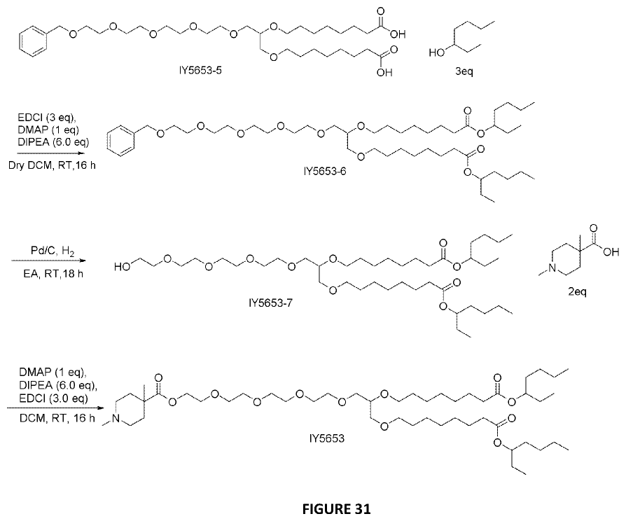

The compound XLII is prepared according to the schema of synthesis of FIG. 31.

### Synthesis of the Intermediate LE-1-IY5653-6

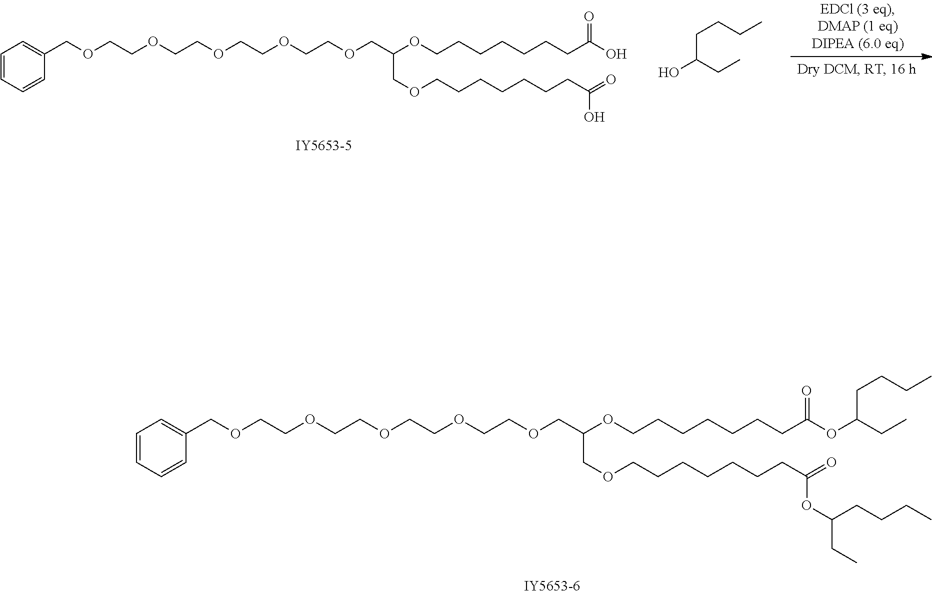

To a solution of heptan-3-ol (0.542 g, 4.67 mmol) and 8-[3-[2-[2-[2-(2-benzyloxyethoxy) ethoxy]ethoxy] ethoxy]-2-(7-carboxyheptoxy) propoxy] octanoic acid (1 g, 1.55 mmol) in dry dichloromethane (20 mL) were added DIPEA (1.01 g, 1.78 mmol), DMAP (0.19 g, 1.55 mmol) and followed by EDCl (1.19 g, 6.22 mmol). The mixture was stirred at room temperature for 16 h. The reaction was diluted with dichloromethane (100 mL) and washed with brine. The organic layer was dried over sodium sulfate, filtered, and concentrated. The residue was purified by flash chromatography eluted with 0% to 4% MeOH in DCM to give 1-ethylpentyl 8-[3-[2-[2-[2-(2-benzyloxyethoxy) ethoxy] ethoxy]ethoxy]-2-[8-(1-ethylpentoxy)-8-oxo-octoxy] propoxy] octanoate (1.15 g, 88.1% yield) as light yellow oil.

1H NMR (400 MHZ, CDCl3) δ 7.40-7.27 (m, 4H), 4.89-4.70 (m, 2H), 4.57 (s, 2H), 3.77-3.25 (m, 24H), 2.28 (t, J=7.5 Hz, 4H), 1.74-1.41 (m, 19H), 1.42-1.11 (m, 21H), 0.97-0.75 (m, 12H).

### Synthesis of the Intermediate LE-1-IY5653-7

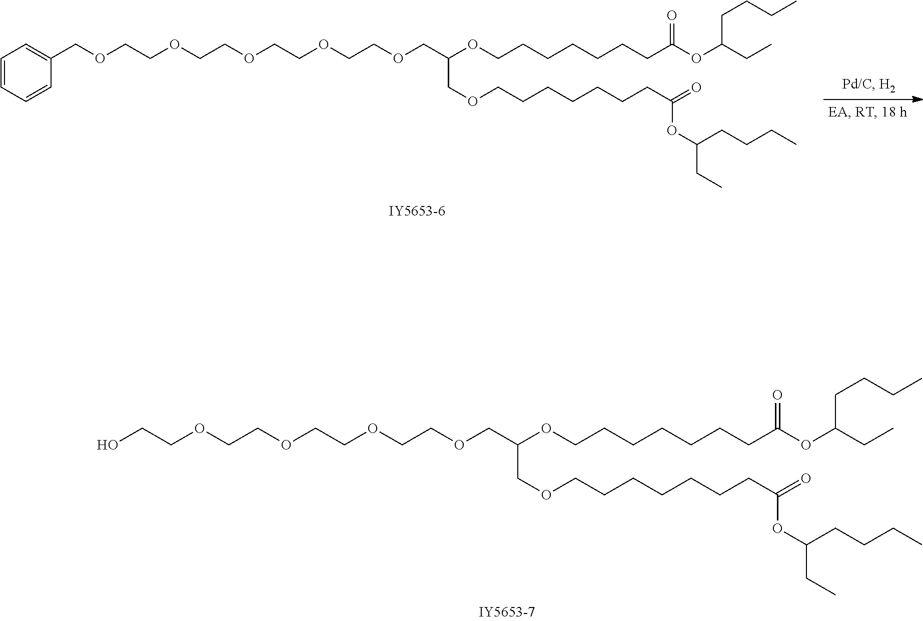 [SMILES: CCCCC(CC)OC(=O)CCCCCCCOCC(COCCOCCOCCOCCOCc1ccccc1)OCCCCCCCC(=O)OC(CC)CCCC ; CCCCC(CC)OC(=O)CCCCCCCOCC(COCCOCCOCCOCCO)OCCCCCCCC(=O)OC(CC)CCCC]

A mixture of 1-ethylpentyl 8-[3-[2-[2-[2-(2-benzyloxyethoxy) ethoxy] ethoxy]ethoxy]-2-[8-(1-ethylpentoxy)-8-oxo-octoxy] propoxy] octanoate (1.15 g, 1.37 mmol) in ethyl acetate (20 mL) was added palladium (292 mg) and through gas H₂, the mixture was stirred at room temperature for 18 hours. Filtered and concentrated to give 1-ethylpentyl 8-[2-[8-(1-ethylpentoxy)-8-oxo-octoxy]-3-[2-[2-[2-(2-hydroxyethoxy) ethoxy] ethoxy]ethoxy] propoxy] octanoate (1 g, yield: 97.4%) as an oil.

1H NMR (400 MHZ, CDCl3) δ 4.94-4.65 (m, 2H), 3.94-3.29 (m, 24H), 2.28 (t, J=7.2 Hz, 4H), 1.68-1.44 (m, 16H), 1.40-1.15 (m, 20H), 1.04-0.73 (m, 12H).

### Synthesis of the Compound XLII

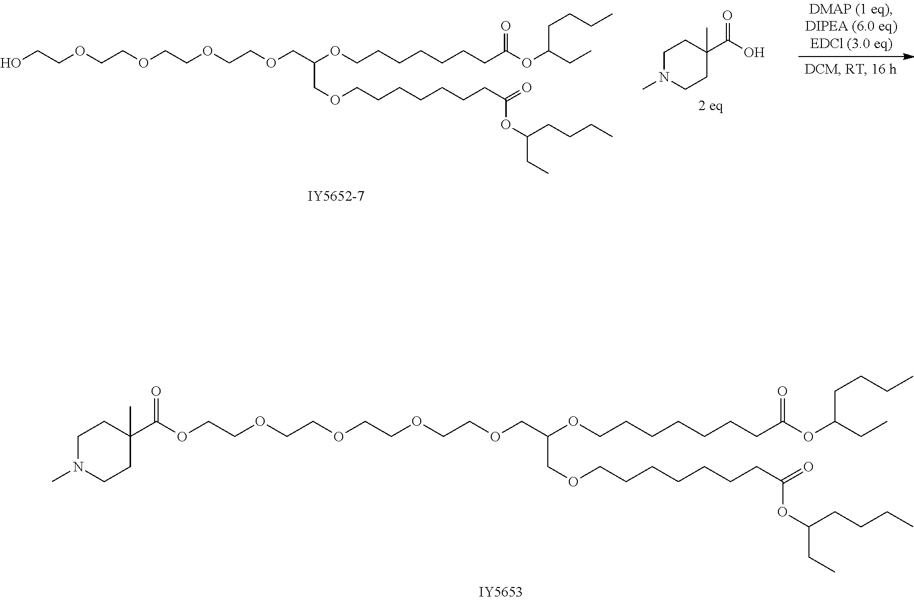

To a solution of 1-ethylpentyl 8-[2-[8-(1-ethylpentoxy)-8-oxo-octoxy]-3-[2-[2-[2-(2-hydroxyethoxy) ethoxy]ethoxy] ethoxy] propoxy] octanoate (0.5 g, 0.66 mmol) and 1,4-dimethylpiperidine-4-carboxylic acid (0.21 g, 1.33 mmol) in dry dichloromethane (10 mL) were added DIPEA (0.431 g, 3.3 mmol), DMAP (0.081 g, 0.66 mmol) and followed by EDCl (0.384 g, 2 mmol). The mixture was stirred at room temperature for 18 h. The reaction was diluted with dichloromethane (50 mL) and washed with brine. The organic layer was dried over sodium sulfate, filtered, and concentrated. The residue was purified by flash chromatography eluted with 0% to 6% MeOH in DCM to give 2-[2-[2-[2-[2,3-bis [8-(1-ethylpentoxy)-8-oxo-octoxy] propoxy] ethoxy]ethoxy] ethoxy]ethyl 1,4-dimethylpiperidine-4-carboxylate (0.325 g, 54.8% yield) as light yellow oil.

1H NMR (400 MHZ, CDCl3) δ 4.98-4.69 (m, 2H), 4.41-4.24 (m, 2H), 3.82-3.25 (m, 22H), 3.04 (s, 2H), 2.51 (s, 4H), 2.38-2.16 (m, 6H), 1.97-1.42 (m, 23H), 1.41-1.11 (m, 25H), 0.99-0.75 (m, 12H).

### Example 35: Synthesis of 1-octylnonyl 8-[3-[2-[2-[2-[2-[3-(4-methylpiperazin-1-yl) propanoyloxy] ethoxy]ethoxy] ethoxy]ethoxy]-2-[8-(1-octylnonoxy)-8-oxo-octoxy] propoxy] octanoate (compound XLIII)

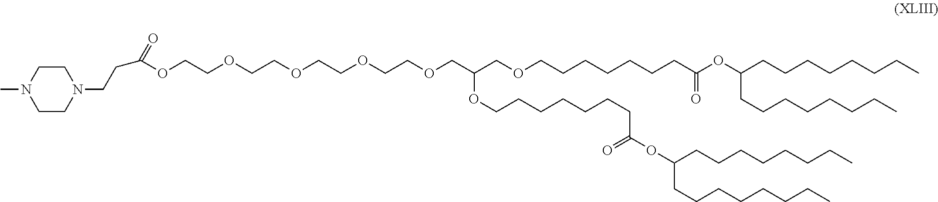 [SMILES: CCCCCCCCC(CCCCCCCC)OC(=O)CCCCCCCOCC(COCCOCCOCCOCCOC(=O)CCN1CCN(C)CC1)OCCCCCCCC(=O)OC(CCCCCCCC)CCCCCCCC]

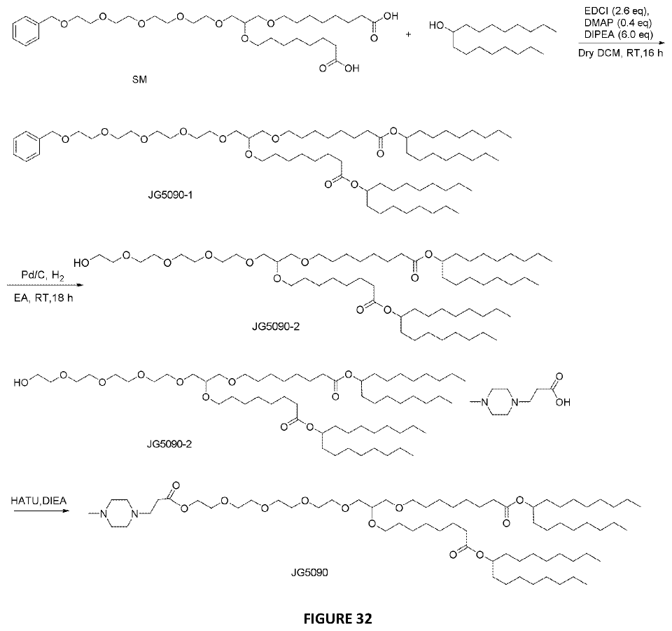

The compound XLIII is prepared according to the schema of synthesis of FIG. 32.

### Synthesis of the Intermediate JG5090-1

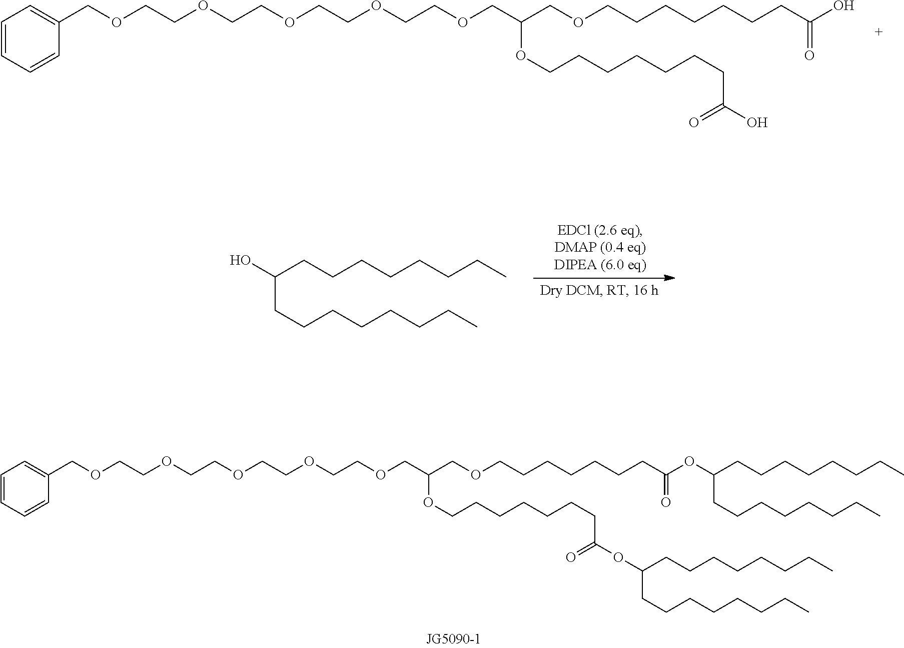 [SMILES: O=C(O)CCCCCCCOCC(COCCOCCOCCOCCOCc1ccccc1)OCCCCCCCC(=O)O ; CCCCCCCCC(O)CCCCCCCC ; CCCCCCCCC(CCCCCCCC)OC(=O)CCCCCCCOCC(COCCOCCOCCOCCOCc1ccccc1)OCCCCCCCC(=O)OC(CCCCCCCC)CCCCCCCC]

To a solution of 8-[3-[2-[2-[2-(2-benzyloxyethoxy) ethoxy]ethoxy] ethoxy]-2-(7-carboxyheptoxy) propoxy] octanoic acid (2 g, 3.11 mmol) and heptadecan-9-ol (3.19 g, 12.4 mmol) in dry dichloromethane (30 mL) were added DIPEA (1.61 g, 12.4 mmol), DMAP (0.38 g, 3.11 mmol) and followed by EDCl (1.79 g, 9.33 mmol). The mixture was stirred at room temperature for 16 h. The reaction was diluted with dichloromethane (150 mL) and washed with brine (100 mL). The organic layer was dried over sodium sulfate, filtered and concentrated. The residue was purified by flash chromatography eluted with 0% to 10% (6%) MeOH in DCM to give 1-octylnonyl 8-[3-[2-[2-[2-(2-benzyloxyethoxy) ethoxy]ethoxy] ethoxy]-2-[8-(1-octylnonoxy)-8-oxo-octoxy] propoxy] octanoate (1.57 g, 45.1% yield) as light yellow oil.

### Synthesis of the Intermediate JG5090-2

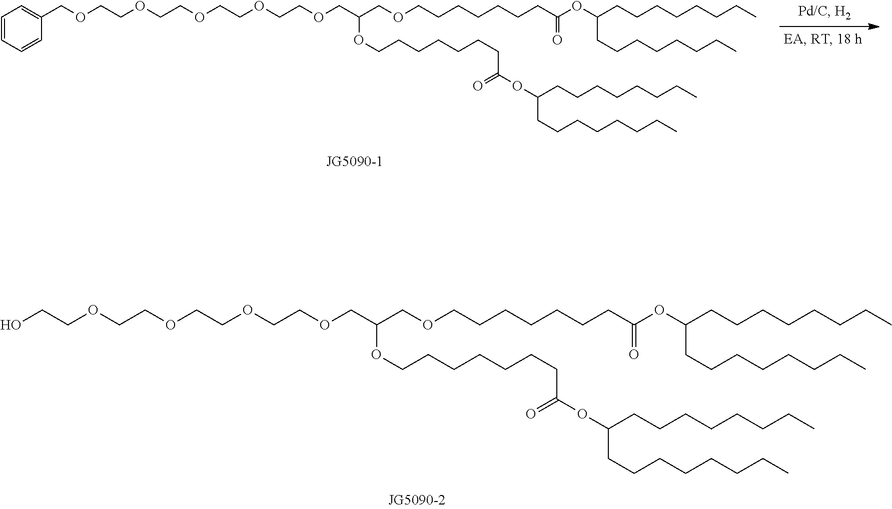 [SMILES: CCCCCCCCC(CCCCCCCC)OC(=O)CCCCCCCOCC(COCCOCCOCCOCCOCc1ccccc1)OCCCCCCCC(=O)OC(CCCCCCCC)CCCCCCCC ; CCCCCCCCC(CCCCCCCC)OC(=O)CCCCCCCOCC(COCCOCCOCCOCCO)OCCCCCCCC(=O)OC(CCCCCCCC)CCCCCCCC]

A mixture of 1-octylnonyl 8-[3-[2-[2-[2-(2-benzyloxyethoxy) ethoxy]ethoxy] ethoxy]-2-[8-(1-octylnonoxy)-8-oxo-octoxy] propoxy] octanoate (1.57 g, 1.4 mmol) in EtOH (30 mL) was added palladium/C (149 mg) and The mixture was stirred at 25° C. for 18 hr under H₂ atmosphere. Filtered and concentrated to give 1-octylnonyl 8-[3-[2-[2-[2-(2-hydroxyethoxy) ethoxy]ethoxy] ethoxy]-2-[8-(1-octylnonoxy)-8-oxo-octoxy] propoxy] octanoate (1.27 g, yield: 88%) as an oil.

### Synthesis of the Compound XLIII

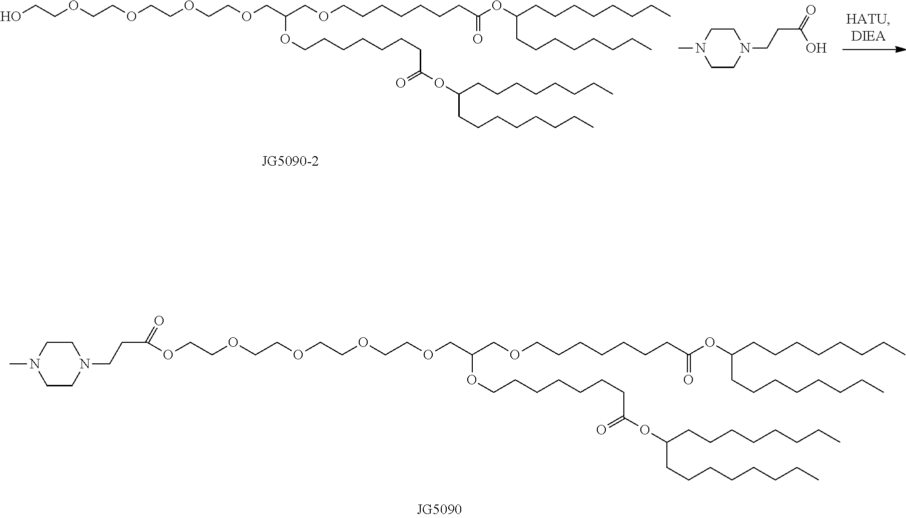

To a solution of 1-octylnonyl 8-[3-[2-[2-[2-(2-hydroxyethoxy) ethoxy]ethoxy] ethoxy]-2-[8-(1-octylnonoxy)-8-oxo-octoxy] propoxy] octanoate (0.5 g, 0.48 mmol) and 3-(4-methylpiperazin-1-yl) propanoic acid (0.24 g, 0.97 mmol) in dry dichloromethane (10 mL) were added DIPEA (0.188 g, 1.46 mmol), DMAP (0.059 g, 0.48 mmol) and followed by EDCl (0.186 g, 0.97 mmol). The mixture was stirred at room temperature for 18 h. The reaction was diluted with dichloromethane (50 mL) and washed with brine (50 mL). The organic layer was dried over sodium sulfate, filtered, and concentrated. The residue was purified by flash chromatography eluted with 0% to 10% (5%) MeOH in DCM to give 1-octylnonyl 8-[3-[2-[2-[2-[2-[3-(4-methylpiperazin-1-yl) propanoyloxy] ethoxy]ethoxy] ethoxy]ethoxy]-2-[8-(1-octylnonoxy)-8-oxo-octoxy] propoxy] octanoate (0.244 g, 42.4% yield) as light yellow oil.

1H NMR (400 MHZ, CDCl3) δ 4.86 (t, J=6.5 Hz, 2H), 4.25 (s, 2H), 3.77-3.34 (m, 21H), 2.84-2.14 (m, 16H), 1.49 (s, 14H), 1.28 (d, J=23.9 Hz, 59H), 0.88 (t, J=6.8 Hz, 12H).

### Example 36: Synthesis of 1-octylnonyl 8-[3-[2-[2-[2-[2-[2-(1-methyl-4-piperidyl) acetyl] oxyethoxy]ethoxy] ethoxy]ethoxy]-2-[8-(1-octylnonoxy)-8-oxo-octoxy] propoxyloctanoate (compound XLIV)

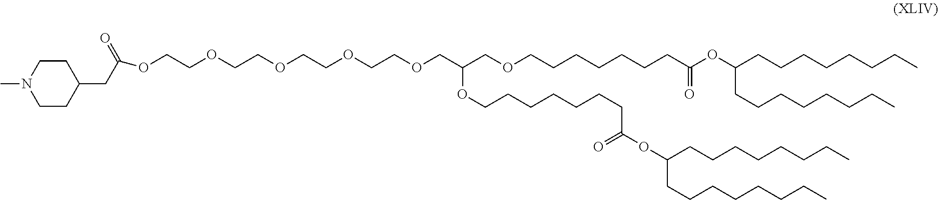 [SMILES: CCCCCCCCC(CCCCCCCC)OC(=O)CCCCCCCOCC(COCCOCCOCCOCCOC(=O)CC1CCN(C)CC1)OCCCCCCCC(=O)OC(CCCCCCCC)CCCCCCCC]

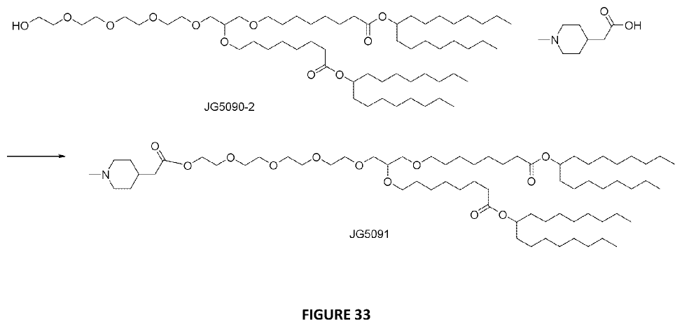

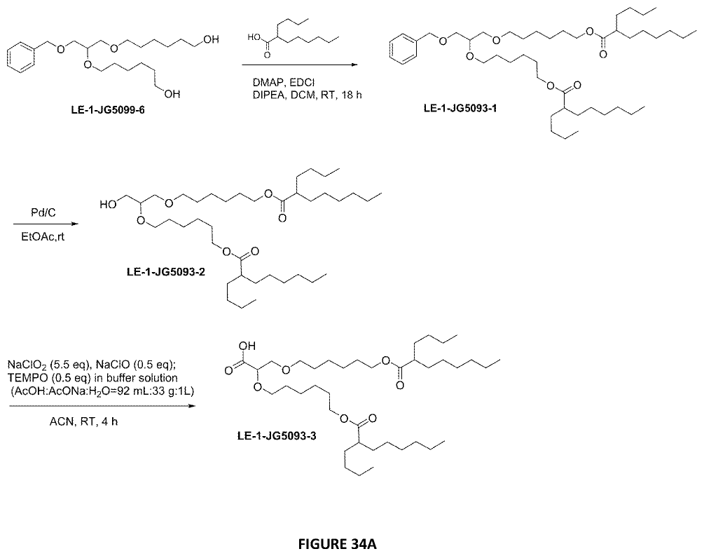

The compound XLIV is prepared according to the schema of synthesis of FIG. 33.

### Synthesis of the Compound XLIV

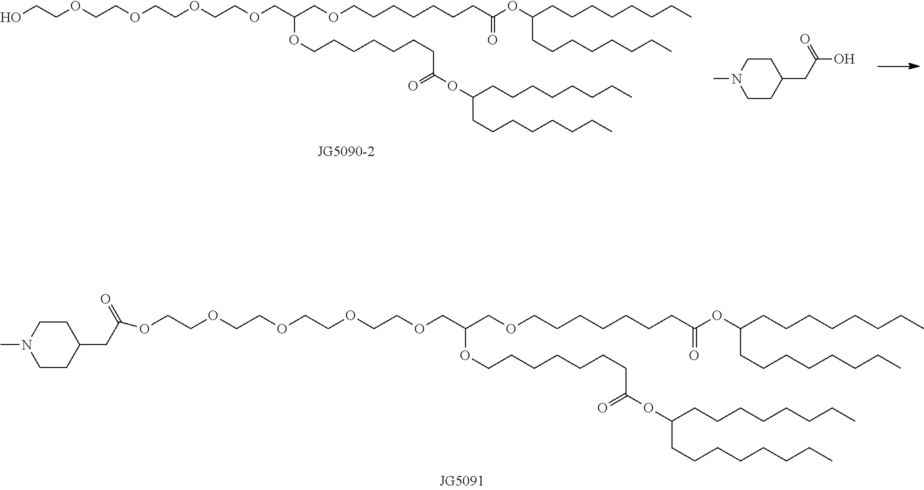

To a solution of 1-octylnonyl 8-[3-[2-[2-[2-(2-hydroxyethoxy) ethoxy]ethoxy] ethoxy]-2-[8-(1-octylnonoxy)-8-oxo-octoxy] propoxy] octanoate (0.5 g, 0.48 mmol) and 2-(1-methyl-4-piperidyl) acetic acid (0.153 g, 0.97 mmol) in dry dichloromethane (10 mL) were added DIPEA (0.188 g, 1.46 mmol), DMAP (0.059 g, 0.48 mmol) and followed by EDCl (0.279 g, 1.46 mmol). The mixture was stirred at room temperature for 18 h. The reaction was diluted with dichloromethane (50 mL) and washed with brine (50 mL). The organic layer was dried over sodium sulfate, filtered, and concentrated. The residue was purified by flash chromatography eluted with 0% to 10% (6%) MeOH in DCM to give 1-octylnonyl 8-[3-[2-[2-[2-[2-[2-(1-methyl-4-piperidyl) acetyl] oxyethoxy]ethoxy] ethoxy]ethoxy]-2-[8-(1-octylnonoxy)-8-oxo-octoxy] propoxy] octanoate (0.256 g, 45.1% yield) as light yellow oil.

1H NMR (400 MHZ, CDCl3) δ 4.94-4.77 (m, 2H), 4.30-4.16 (m, 2H), 3.77-3.35 (m, 21H), 3.07 (s, 2H), 2.45 (s, 3H), 2.38-2.19 (m, 7H), 1.81 (d, J=12.1 Hz, 4H), 1.51 (s, 12H), 1.38-1.12 (m, 62H), 0.88 (t, J=6.8 Hz, 12H).

### Example 37: Synthesis of 2-[2-[2-[2-[2-[2,3-bis [6-(2-butyloctanoyloxy) hexoxy] propanoyl-octyl-amino]ethoxy]ethoxy] ethoxy]ethoxy]ethyl 1-methylpiperidine-4-carboxylate (compound XLV)

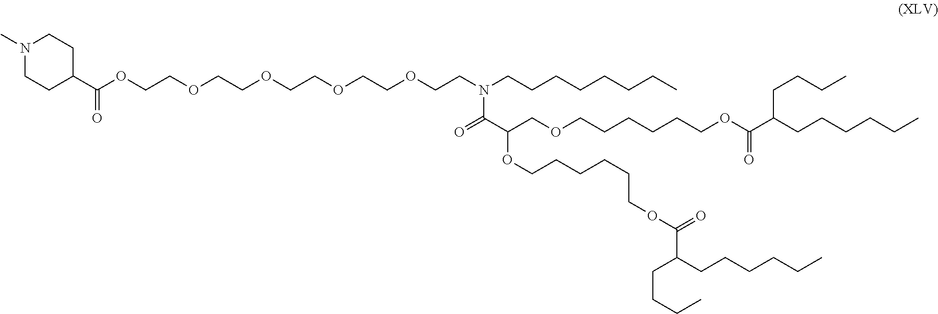 [SMILES: CCCCCCCCN(CCOCCOCCOCCOCCOC(=O)C1CCN(C)CC1)C(=O)C(COCCCCCCOC(=O)C(CCCC)CCCCCC)OCCCCCCOC(=O)C(CCCC)CCCCCC]

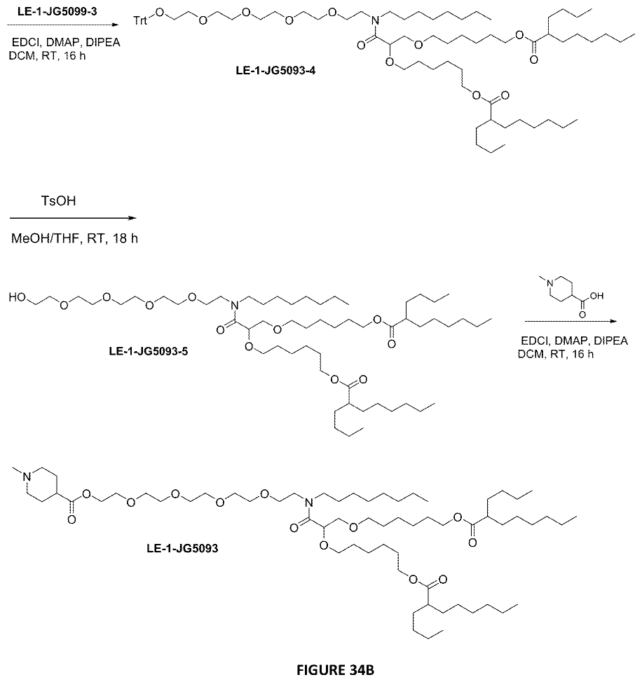

The compound XLV is prepared according to the schema of synthesis of FIGS. 34A and 34B.

### Synthesis of the Intermediate LE-1-JG5093-1

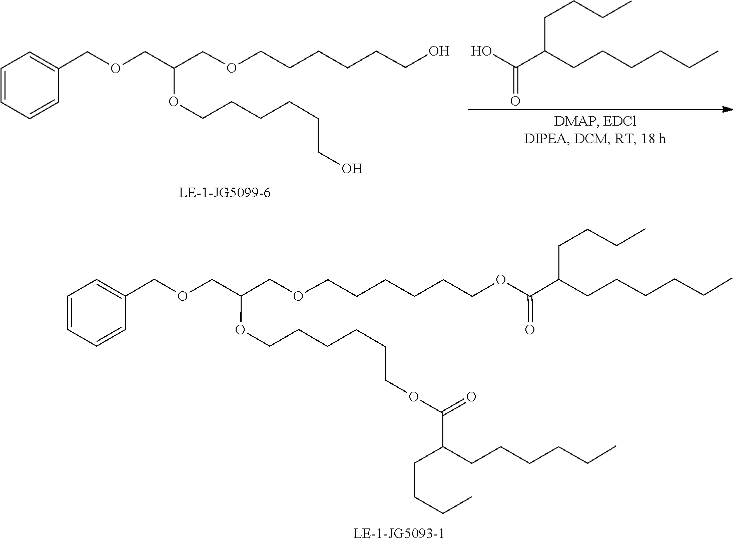 [SMILES: OCCCCCCOCC(COCc1ccccc1)OCCCCCCO ; CCCCCCC(CCCC)C(=O)O ; CCCCCCC(CCCC)C(=O)OCCCCCCOCC(COCc1ccccc1)OCCCCCCOC(=O)C(CCCC)CCCCCC]

To a solution of 6-[3-benzyloxy-2-(6-hydroxyhexoxy) propoxy] hexan-1-ol (1 g, 2.61 mmol) in DCM (50 ml) was added 2-butyloctanoic acid (1.57 g, 7.84 mmol),N-ethyl-N-isopropyl-propan-2-amine (1.01 g, 7.84 mmol), N,N-dimethylpyridin-4-amine (0.032 g, 0.26 mmol), 3-(ethyliminomethyleneamino)-N,N-dimethyl-propan-1-amine; hydrochloride (1.5 g, 2.61 mmol). The mixture was stirred at room temperature for 18 hr. The mixture was added DCM (100 mL) and washed with 0.5 N HCl (50 ml), NaHCO₃ (50 ml), NaCl (50 ml). The organic was concentrated and the residue was purified by flash column chromatography on silica gel eluting with 1:2 ethyl acetate/petroleum ether to give 6-[3-benzyloxy-2-[6-(2-butyloctanoyloxy) hexoxy] propoxy] hexyl 2-butyloctanoate (1.7 g, 87%) as colorless oil.

1H NMR (400 MHZ, CDCl3) δ 7.37-7.27 (m, 4H), 4.55 (s, 1H), 4.06 (td, J=6.6, 1.7 Hz, 3H), 3.52 (dddd, J=32.0, 30.3, 9.5, 5.1 Hz, 7H), 2.37-2.24 (m, 2H), 1.72-1.52 (m, 11H), 1.45-1.18 (m, 34H), 0.91-0.84 (m, 12H).

### Synthesis of the Intermediate LE-1-JG5093-2

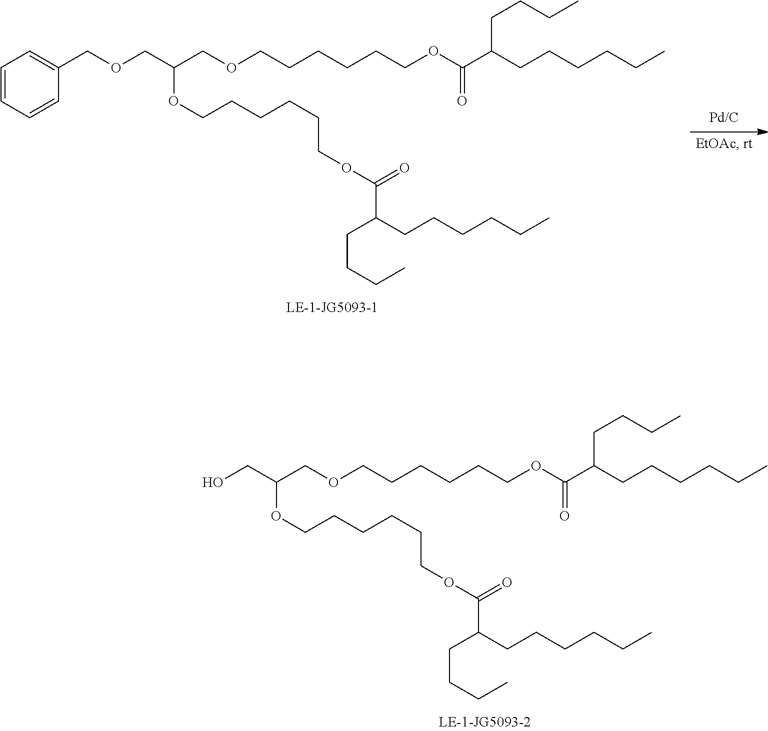 [SMILES: CCCCCCC(CCCC)C(=O)OCCCCCCOCC(COCc1ccccc1)OCCCCCCOC(=O)C(CCCC)CCCCCC ; "EtOAc, rt" ; CCCCCCC(CCCC)C(=O)OCCCCCCOCC(CO)OCCCCCCOC(=O)C(CCCC)CCCCCC]

A solution of 6-[3-benzyloxy-2-[6-(2-butyloctanoyloxy) hexoxy] propoxy] hexyl 2-butyloctanoate (1.7 g, 2.28 mmol) and Pd/C (0.5 g) were mixed with EtOAc (50 mL) and attached to a hydrogenation apparatus. The system was evacuated and then refilled with hydrogen. The reaction mixture was stirred for 16 h at room temperature. The mixture was filtered and the filtrate was concentrated to give 6-[2-[6-(2-butyloctanoyloxy) hexoxy]-3-hydroxy-propoxy] hexyl 2-butyloctanoate (1.5 g, 100%) as colorless oil.

1H NMR (400 MHZ, CDCl3) δ 4.07 (t, J=6.6 Hz, 3H), 3.76-3.70 (m, 1H), 3.65-3.40 (m, 7H), 2.38-2.26 (m, 3H), 1.68-1.54 (m, 11H), 1.49-1.22 (m, 36H), 0.88 (tt, J=7.0, 3.5 Hz, 12H).

### Synthesis of the Intermediate LE-1-JG5093-3

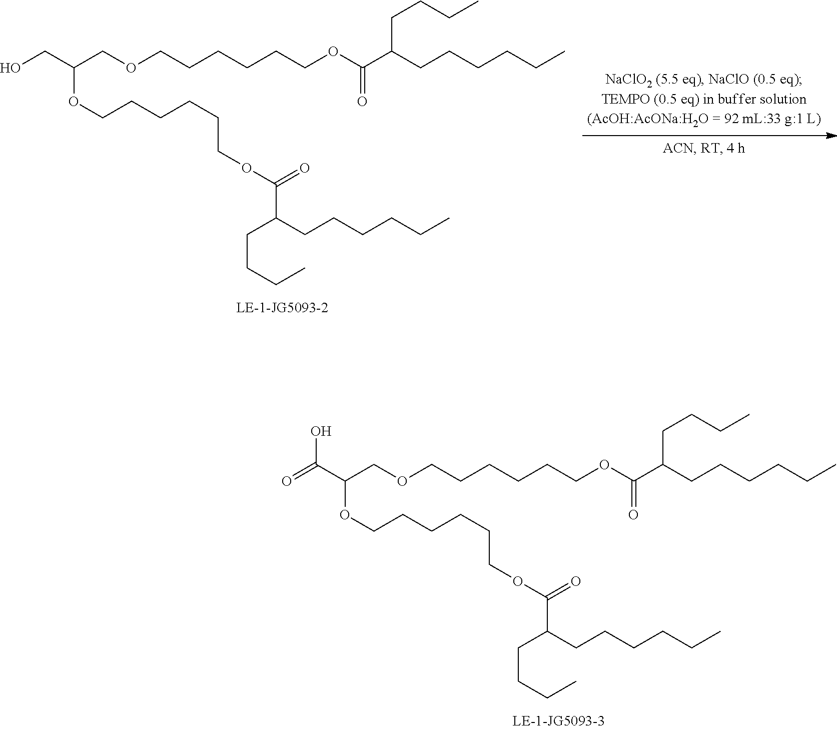 [SMILES: CCCCCCC(CCCC)C(=O)OCCCCCCOCC(CO)OCCCCCCOC(=O)C(CCCC)CCCCCC ; "ACN, RT, 4 h" ; CCCCCCC(CCCC)C(=O)OCCCCCCOCC(OCCCCCCOC(=O)C(CCCC)CCCCCC)C(=O)O]

To a solution of 6-[2-[6-(2-butyloctanoyloxy) hexoxy]-3-hydroxy-propoxy] hexyl 2-butyloctanoate (1.5 g, 2.28 mmol) in Acetonitrile (AcCN, 50 mL) and PH=4-buffer solution (10 mL, AcOH: AcONa: water=92 mL: 33 g: 1000 mL) were added sodium chlorite (1.03 g, 11.4 mmol) and sodium hypochlorite (0.17 g, 1.14 mmol) and followed by TEMPO ((2,2,6,6-tetramethylpiperidin-1-yl)oxyl) (0.178 g, 1.14 mmol). The reaction became black and stirred at 20° C. for 4 hr. LCMS indicated a clean reaction. The reaction was quenched with 20 drops of methanol and was poured into water (40 mL) and extracted with ethyl acetate. The organic layers were combined, washed with brine (60 mL), dried over anhydrous sodium sulfate, filtered, and concentrated. The residue was purified through flash chromatography eluted with 5% to 10% methanol in dichloromethane to give 2,3-bis [6-(2-butyloctanoyloxy) hexoxy] propanoic acid (1.2 g, 78.3% yield) as light yellow oil.

1H NMR (400 MHZ, CDCl3) δ 4.14-4.00 (m, 5H), 3.83-3.39 (m, 6H), 2.31 (dq, J=9.0, 5.3 Hz, 2H), 1.72-1.52 (m, 12H), 1.51-1.17 (m, 37H), 0.88 (td, J=6.9, 2.2 Hz, 12H).

### Synthesis of the Intermediate LE-1-JG5093-4

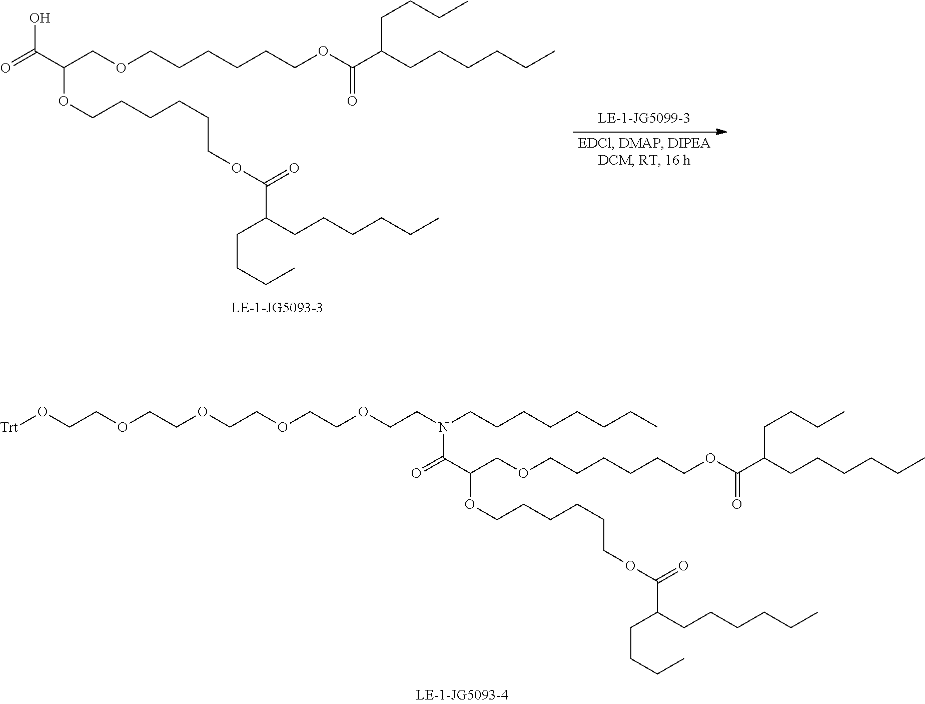

To a mixture of 2,3-bis [6-(2-butyloctanoyloxy) hexoxy] propanoic acid (1.2 g, 1.79 mmol), EDC HCl (0.686 g, 3.58 mmol), DIEA (0.462 g, 3.58 mmol) in DCM (20 mL) were added N-[2-[2-[2-[2-(2-trityloxyethoxy) ethoxy]ethoxy] ethoxy]ethyl] octan-1-amine (1.16 g, 1.97 mmol) and N,N-dimethylpyridin-4-amine (0.43 g, 0.36 mmol). The mixture was stirred for 16 h at RT. The mixture was poured into DCM (200 mL) and washed with NaHCO₃ solution and brine, dried over Na2SO4 and concentrated. The residue was purified by flash column chromatography on silica gel eluting with 1:3 ethyl acetate/petroleum ether to give 6-[2-[6-(2-butyloctanoyloxy) hexoxy]-3-[octyl-[2-[2-[2-[2-(2-trityloxyethoxy) ethoxy] ethoxy]ethoxy]ethyl]amino]-3-oxo-propoxy] hexyl 2-butyloctanoate (1.2 g, 53.9%) as colorless oil.

1H NMR (400 MHZ, CDCl3) δ 7.46 (d, J=7.3 Hz, 6H), 7.34-7.26 (m, 7H), 7.22 (t, J=7.2 Hz, 3H), 4.37 (dt, J=11.4, 5.9 Hz, 1H), 4.05 (t, J=5.8 Hz, 4H), 3.77-3.18 (m, 31H), 2.38-2.24 (m, 2H), 1.74-1.02 (m, 73H), 0.87 (dd, J=7.8, 6.0 Hz, 16H).

### Synthesis of the Intermediate LE-1-JG5093-5

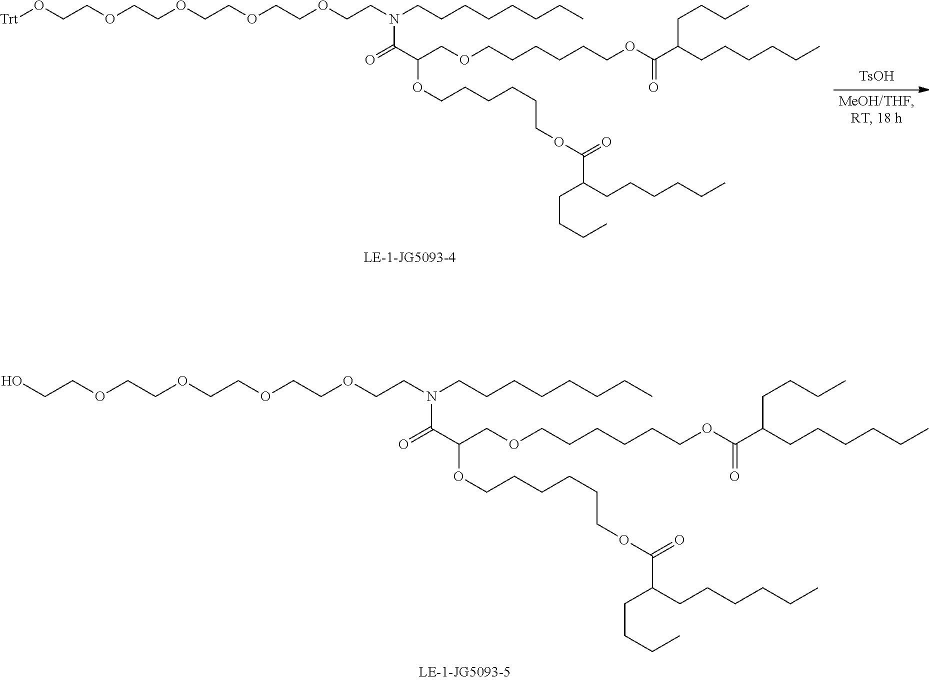

To a solution of 6-[2-[6-(2-butyloctanoyloxy) hexoxy]-3-[octyl-[2-[2-[2-[2-(2-trityloxyethoxy) ethoxy]ethoxy] ethoxy]ethyl]amino]-3-oxo-propoxy] hexyl 2-butyloctanoate (1.2 g, 0.848 mmol) in methanol/THF (20 mL, 1/1 v/v) was added 4-methylbenzenesulfonic acid (0.498 g, 2.89 mmol) in one portion at room temperature and the mixture was stirred at room temperature for 18 h. TLC (4% ethyl acetate in petroleum ether) indicated that the starting material was disappeared completely. 2 mL triethylamine was added to quench the reaction and the solvent was removed under vacuum. The residue was purified by flash chromatography eluted with 0% to 5% MeOH in DCM (4%) to give 6-[2-[6-(2-butyloctanoyloxy) hexoxy]-3-[2-[2-[2-[2-(2-hydroxyethoxy) ethoxy]ethoxy] ethoxy]ethyl-octyl-amino]-3-oxo-propoxy] hexyl 2-butyloctanoate (0.85 g, 88% yield) as colorless oil.

1H NMR (400 MHZ, CDCl3) δ 4.47-4.27 (m, 1H), 4.05 (td, J=6.6, 2.1 Hz, 4H), 3.82-3.23 (m, 29H), 2.40-2.23 (m, 2H), 2.05 (s, 3H), 1.75-1.17 (m, 62H), 0.87 (td, J=6.9, 2.3 Hz, 15H).

### Synthesis of the Compound XLV

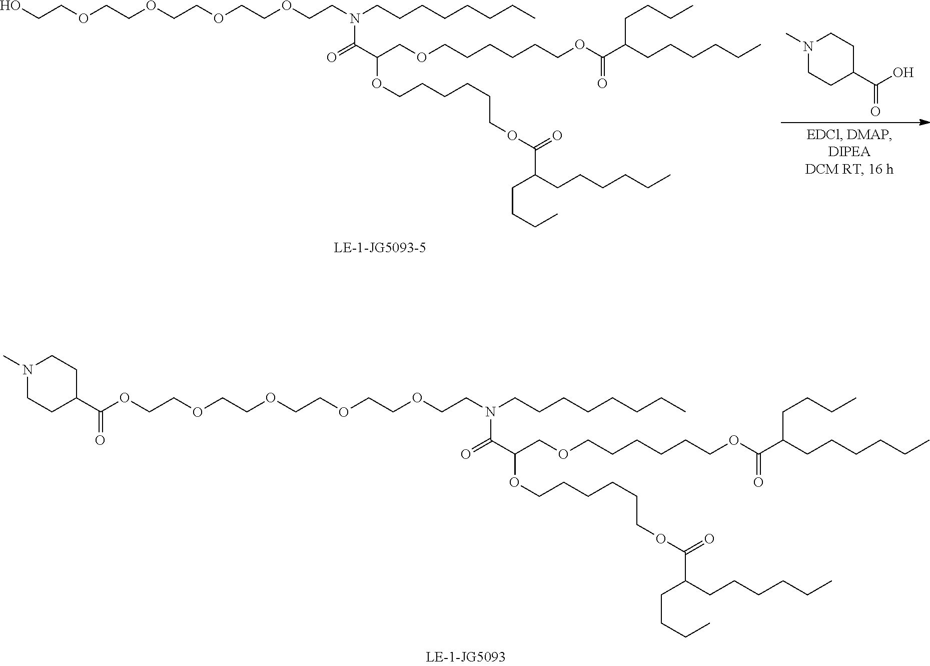

To the solution of 6-[2-[6-(2-butyloctanoyloxy) hexoxy]-3-[2-[2-[2-[2-(2-hydroxyethoxy) ethoxy]ethoxy] ethoxy]ethyl-octyl-amino]-3-oxo-propoxy] hexyl 2-butyloctanoate (0.85 g, 0.848 mmol) and 1-methylpiperidine-4-carboxylic acid (0.364 g, 2.54 mmol) in dry dichloromethane (20 mL) were added DIPEA (0.329 g, 2.54 mmol), DMAP (0.02 g, 1.7 mmol) and followed by under ice bath EDCl (0.488 g, 2.54 mmol) portionwise. The mixture was stirred at room temperature for 16 h. The reaction was diluted with dichloromethane and washed with brine. The organic layer was dried over sodium sulfate, filtered and concentrated. The residue was purified by flash chromatography eluted with 0% to 10% methanol in dichloromethane to give 2-[2-[2-[2-[2-[2,3-bis [6-(2-butyloctanoyloxy) hexoxy] propanoyl-octyl-amino]ethoxy]ethoxy] ethoxy]ethoxy]ethyl 1-methylpiperidine-4-carboxylate (0.618 g, 64.6% yield) as colorless oil.

1H NMR (400 MHZ, CDCl3) δ 4.47-4.29 (m, 1H), 4.27-4.19 (m, 2H), 4.05 (td, J=6.6, 2.0 Hz, 4H), 3.79-3.24 (m, 26H), 2.83 (d, J=11.3 Hz, 2H), 2.38-2.24 (m, 6H), 2.10-1.89 (m, 4H), 1.87-1.70 (m, 6H), 1.67-1.54 (m, 13H), 1.48-1.21 (m, 46H), 0.87 (td, J=6.9, 2.2 Hz, 15H).

### Example 38: Synthesis of 2-[2-[2-[2-[2-[2,3-bis [6-oxo-6-(3-pentyloctoxy) hexoxy] propanoyl-octyl-amino]ethoxy]ethoxy] ethoxy]ethoxy]ethyl 1-methylpiperidine-4-carboxylate (compound XLVI)

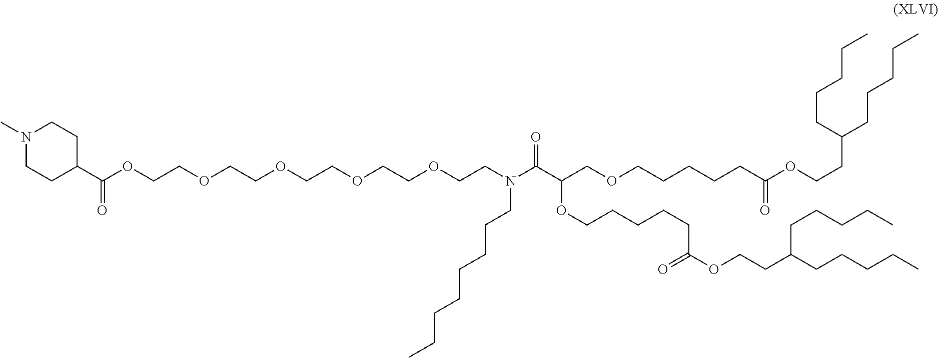 [SMILES: CCCCCCCCN(CCOCCOCCOCCOCCOC(=O)C1CCN(C)CC1)C(=O)C(COCCCCCC(=O)OCCC(CCCCC)CCCCC)OCCCCCC(=O)OCCC(CCCCC)CCCCC]

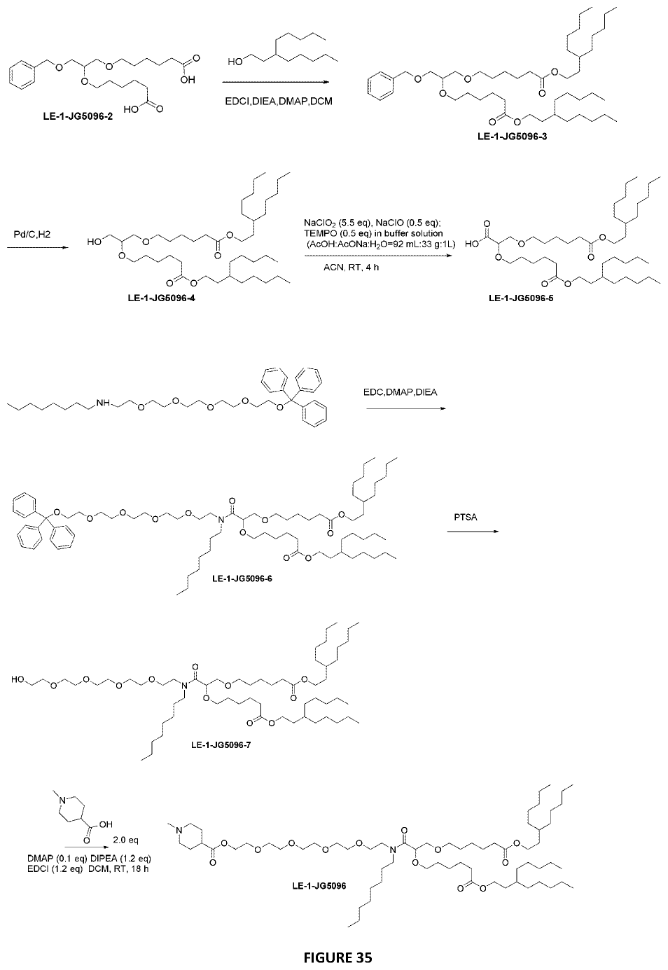

The compound XLVI is prepared according to the schema of synthesis of FIG. 35.

### Synthesis of the Intermediate LE-1-JG5096-3

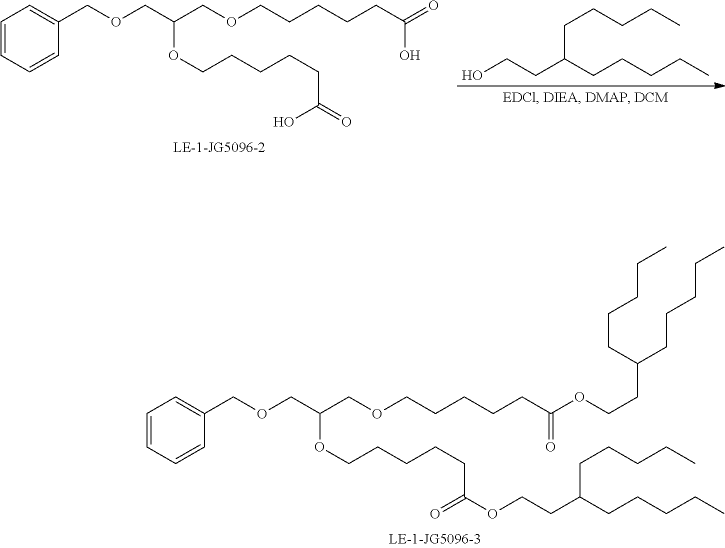

To a solution of 6-[3-benzyloxy-2-(5-carboxypentoxy) propoxy] hexanoic acid (1.0 g, 2.44 mmol) and 3-pentyloctan-1-ol (1.46 g, 7.3 mmol) in dry dichloromethane (20 mL) were added DIPEA (0.945 g, 7.31 mmol), DMAP (0.298 g, 2.44 mmol) and followed by EDCl (0.934 g, 4.87 mmol). The mixture was stirred at room temperature for 18 h. The reaction was diluted with dichloromethane and washed with brine. The organic layer was dried over sodium sulfate, filtered and concentrated. The residue was purified by flash PE eluted with 0% to 10% (7%) in EA to give 3-pentyloctyl 6-[3-benzyloxy-2-[6-oxo-6-(3-pentyloctoxy)hexoxy] propoxy] hexanoate (1.69 g, yield 84.7%) as light yellow oil.

### Synthesis of the Intermediate LE-1-JG5096-4

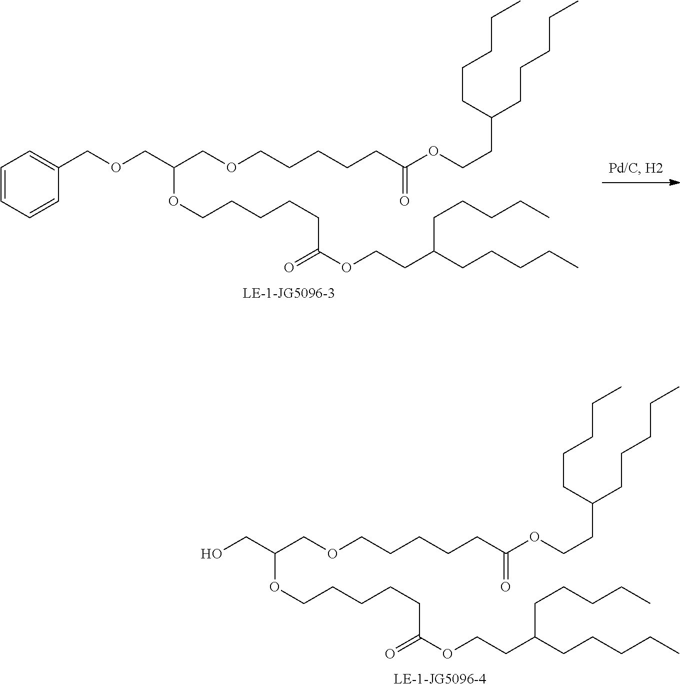 [SMILES: CCCCCC(CCCCC)CCOC(=O)CCCCCOCC(COCc1ccccc1)OCCCCCC(=O)OCCC(CCCCC)CCCCC ; CCCCCC(CCCCC)CCOC(=O)CCCCCOCC(CO)OCCCCCC(=O)OCCC(CCCCC)CCCCC]

A mixture of 3-pentyloctyl 6-[3-benzyloxy-2-[6-oxo-6-(3-pentyloctoxy) hexoxy] propoxy] hexanoate (1.6 g, 2.06 mmol) in MeOH (20 mL) was added palladium (220 mg) and through gas H₂, the mixture was stirred at room temperature for 16 hours. Filtered and concentrated to give 3-pentyloctyl 6-[3-hydroxy-2-[6-oxo-6-(3-pentyloctoxy) hexoxy] propoxy] hexanoate (1.40 g, yield: 99%) as an oil.

1H NMR (400 MHZ, CDCl3) δ 4.10 (t, J=7.1 Hz, 4H), 3.74 (s, 1H), 3.68-3.37 (m, 9H), 2.37-2.19 (m, 5H), 1.78-1.58 (m, 27H), 1.58-1.17 (m, 43H), 0.90 (t, J=7.0 Hz, 12H).

### Synthesis of the Intermediate LE-1-JG5096-5

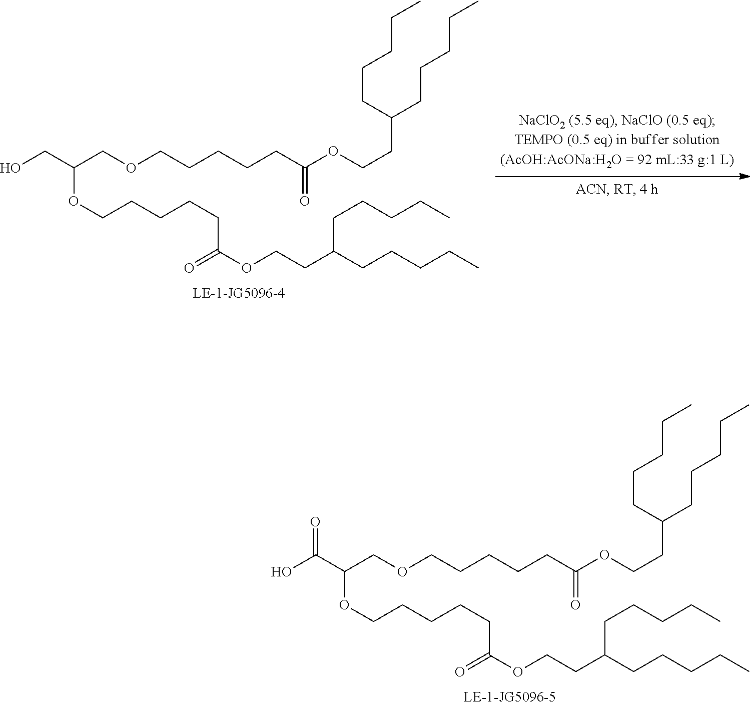 [SMILES: CCCCCC(CCCCC)CCOC(=O)CCCCCOCC(CO)OCCCCCC(=O)OCCC(CCCCC)CCCCC ; "ACN, RT, 4 h" ; CCCCCC(CCCCC)CCOC(=O)CCCCCOCC(OCCCCCC(=O)OCCC(CCCCC)CCCCC)C(=O)O]

To a solution of 3-pentyloctyl 6-[3-hydroxy-2-[6-oxo-6-(3-pentyloctoxy) hexoxy] propoxy] hexanoate (1.4 g, 2.04 mmol) in Acetonitrile (AcCN, 20 mL) and PH=4-buffer solution (10 mL, AcOH: AcONa: water=92 mL: 33 g: 1000 mL) were added sodium chlorite (1.02 g, 11.2 mmol) and sodium hypochlorite (0.0726 g, 0.975 mmol) and followed by TEMPO ((2,2,6,6-tetramethylpiperidin-1-yl)oxyl) (0.152 g, 0.0975 mmol). The reaction became black and stirred at 20° C. for 4 hr. LCMS indicated a clean reaction. The reaction was quenched with 20 drops of methanol and was poured into water (40 mL) and extracted with ethyl acetate (60 ml×3). The organic layers were combined, washed with brine (60 mL), dried over anhydrous sodium sulfate, filtered and concentrated. The residue, combined with above batch, was purified through flash chromatography eluted with 5% to 10% methanol in dichloromethane to give 2,3-bis [6-oxo-6-(3-pentyloctoxy) hexoxy] propanoic acid (1.403 g, 98% yield) as light yellow oil.

1H NMR (400 MHZ, CDCl3) δ 7.26 (s, 6H), 4.16-3.99 (m, 5H), 3.82-3.36 (m, 6H), 2.30 (td, J=7.5, 3.6 Hz, 4H), 2.10 (s, 1H), 2.05 (s, 1H), 1.62 (ddt, J=27.0, 13.8, 6.8 Hz, 17H), 1.46-1.15 (m, 42H), 0.88 (t, J=7.0 Hz, 12H).

### Synthesis of the Intermediate LE-1-JG5096-6

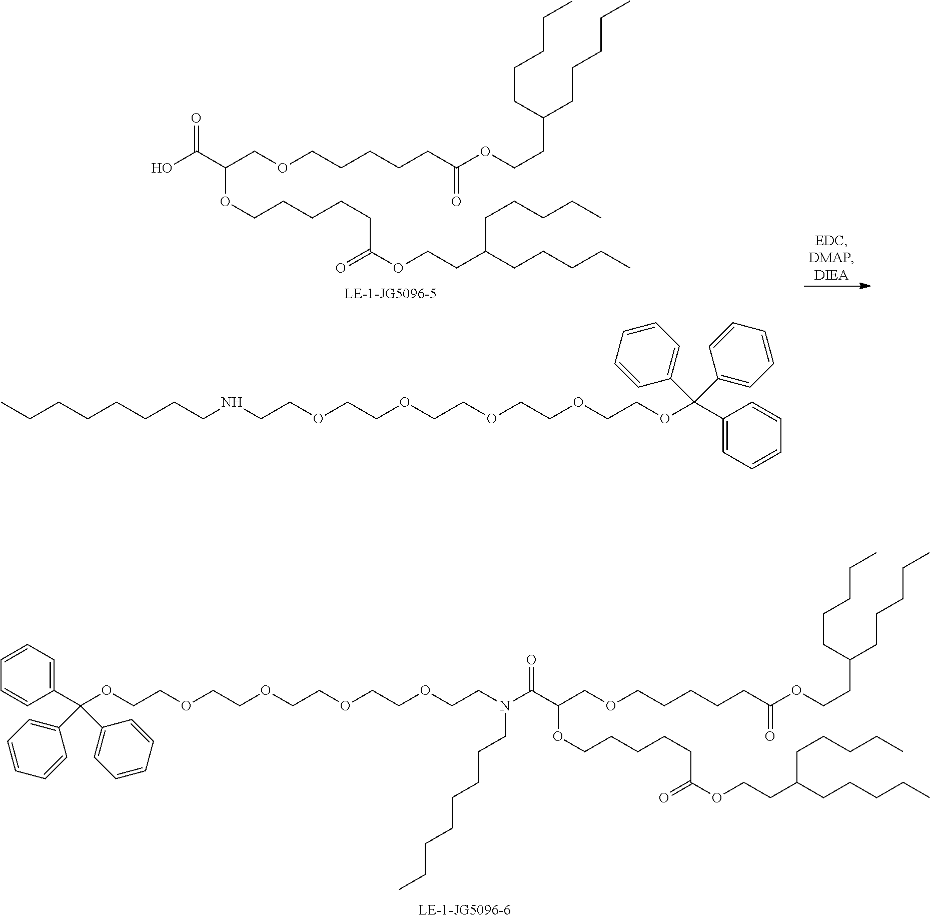 [SMILES: CCCCCC(CCCCC)CCOC(=O)CCCCCOCC(OCCCCCC(=O)OCCC(CCCCC)CCCCC)C(=O)O ; "EDC,DMAP,DIEA" ; CCCCCCCCNCCOCCOCCOCCOCCOC(c1ccccc1)(c1ccccc1)c1ccccc1 ; CCCCCCCCN(CCOCCOCCOCCOCCOC(c1ccccc1)(c1ccccc1)c1ccccc1)C(=O)C(COCCCCCC(=O)OCCC(CCCCC)CCCCC)OCCCCCC(=O)OCCC(CCCCC)CCCCC]

To a solution of N-[2-[2-[2-[2-(2-trityloxyethoxy) ethoxy]ethoxy] ethoxy]ethyl] octan-1-amine (1.02 g, 1.72 mmol) in DCM (20 ml) were added 2,3-bis [6-oxo-6-(3-pentyloctoxy) hexoxy] propanoic acid (1 g, 1.43 mmol), 3-(ethyliminomethyleneamino)-N,N-dimethyl-propan-1-amine;hydrochloride (548 mg, 2.25 mmol), DMAP (175 mg, 1.43 mmol) and N-ethyl-N-isopropyl-propan-2-amine (555 mg, 4.29 mmol). The mixture was stirred at 25° C. for 18 hr. Then the mixture was concentrated and dealt with EA (50 ml), washed with water (50 ml×2), brine (50 ml) and dried over Na2SO4. The organic was concentrated and purified by flash chromatography column (40% EA in PE) to give 3-pentyloctyl 6-[3-[octyl-[2-[2-[2-[2-(2-trityloxyethoxy)ethoxy]ethoxy] ethoxy]ethyl]amino]-3-oxo-2-[6-oxo-6-(3-pentyloctoxy)hexoxy] propoxy] hexanoate (1.2 g, 0.94 mmol, yield 65.9%) as a colorless oil.

1H NMR (400 MHZ, CDCl3) δ 7.46 (d, J=7.4 Hz, 5H), 7.34-7.18 (m, 13H), 5.30 (s, 1H), 4.16-4.02 (m, 3H), 3.79-3.49 (m, 16H), 3.49-3.34 (m, 5H), 3.25 (dd, J=14.7, 9.4 Hz, 2H), 2.28 (t, J=7.6 Hz, 3H), 1.61 (d, J=8.3 Hz, 24H), 1.58-1.07 (m, 47H), 0.88 (t, J=6.9 Hz, 12H).

### Synthesis of the Intermediate LE-1-JG5096-7

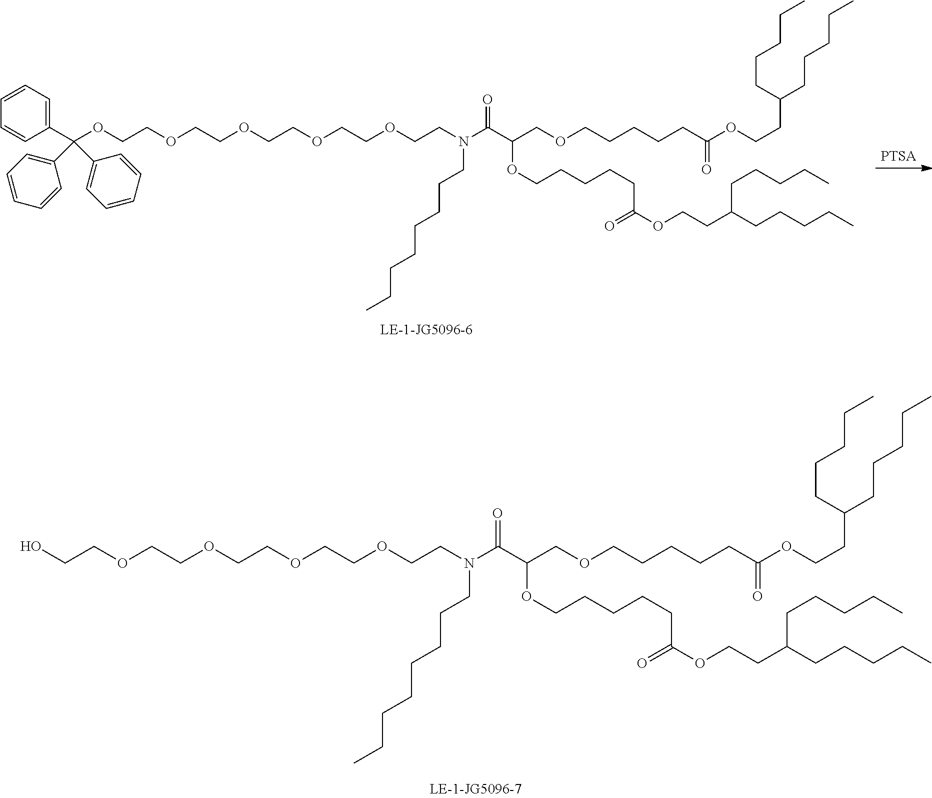 [SMILES: CCCCCCCCN(CCOCCOCCOCCOCCOC(c1ccccc1)(c1ccccc1)c1ccccc1)C(=O)C(COCCCCCC(=O)OCCC(CCCCC)CCCCC)OCCCCCC(=O)OCCC(CCCCC)CCCCC ; "PTSA" ; CCCCCCCCN(CCOCCOCCOCCOCCO)C(=O)C(COCCCCCC(=O)OCCC(CCCCC)CCCCC)OCCCCCC(=O)OCCC(CCCCC)CCCCC]

A mixture of 3-pentyloctyl 6-[3-[octyl-[2-[2-[2-[2-(2-trityloxyethoxy) ethoxy]ethoxy] ethoxy]ethyl]amino]-3-oxo-2-[6-oxo-6-(3-pentyloctoxy) hexoxy] propoxy] hexanoate (0.5 g, 0.39 mmol) and 4-methylbenzenesulfonic acid/hydrate (448 mg, 2.36 mmol) in tetrahydrofuran (10 mL). The mixture was stirred for 16 h at ambient temperature. The mixture was concentrated and the residue was purified by column chromatography on silica gel eluting with 0% ˜10% MeOH in DCM to afford 3-pentyloctyl 6-[3-[2-[2-[2-[2-(2-hydroxyethoxy) ethoxy]ethoxy] ethoxy]ethyl-octyl-amino]-3-oxo-2-[6-oxo-6-(3-pentyloctoxy) hexoxy] propoxy] hexanoate (0.35 g, 86.5% yield) as yellow oil.

1H NMR (400 MHZ, CDCl3) δ 4.07 (dd, J=7.1, 5.4 Hz, 3H), 3.73 (d, J=4.3 Hz, 1H), 3.72-3.30 (m, 21H), 2.28 (t, J=7.6 Hz, 3H), 1.70-1.25 (m, 68H), 1.25-1.13 (m, 14H), 0.88 (t, J=7.0 Hz, 12H).

### Synthesis of the Compound XLVI

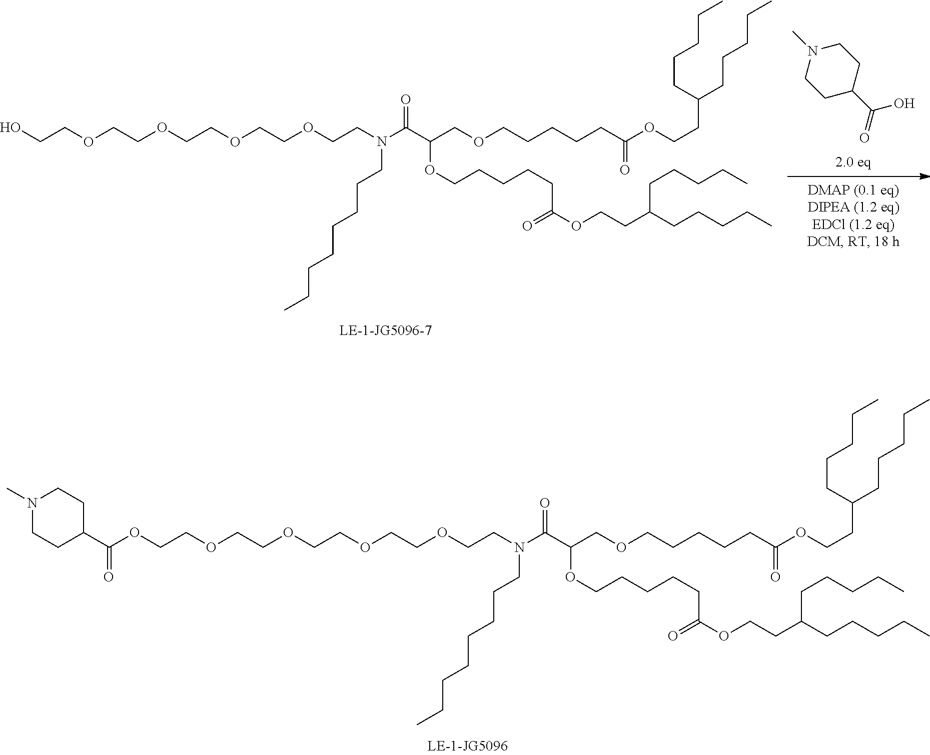

To a solution of 6-[3-[2-[2-[2-[2-(2-3-pentyloctyl hydroxyethoxy) ethoxy]ethoxy] ethoxy]ethyl-octyl-amino]-3-oxo-2-[6-oxo-6-(3-pentyloctoxy) hexoxy] propoxy] hexanoate (0.35 g, 0.4 mmol) and 1-methylpiperidine-4-carboxylic acid (97 mg, 0.67 mmol) in dry dichloromethane (10 mL) were added DIPEA (132 mg, 1.02 mmol), DMAP (41.5 mg, 0.34 mmol) and followed by EDCl (130 mg, 0.67 mmol). The mixture was stirred at room temperature for 18 h. The reaction was diluted with dichloromethane and washed with brine. The organic layer was dried over sodium sulfate, filtered and concentrated. The residue was purified by flash chromatography eluted with 0% 10% (7%) MeOH in DCM to give to 2-[2-[2-[2-[2-[2,3-bis [6-oxo-6-(3-pentyloctoxy) hexoxy] propanoyl-octyl-amino]ethoxy]ethoxy] ethoxy]ethoxy]ethyl 1-methylpiperidine-4-carboxylate (0.359 g, 91.5% yield) as light yellow oil.

1H NMR (400 MHZ, CDCl3) δ 4.47-4.16 (m, 2H), 4.07 (t, J=6.3 Hz, 3H), 3.75-3.21 (m, 20H), 2.84 (s, 1H), 2.29 (dd, J=15.2, 7.6 Hz, 6H), 1.84 (s, 4H), 1.59-1.48 (m, 10H), 1.47-1.12 (m, 42H), 0.88 (t, J=7.0 Hz, 12H).

### Example 39: Synthesis of nonyl 8-[3-[2-[2-[2-[2-[(1-methylpiperidine-4-carbonyl) amino]ethoxy]ethoxy] ethoxy]ethoxy]-2-(8-nonoxy-8-oxooctoxy) propoxyloctanoate (compound XXIV)

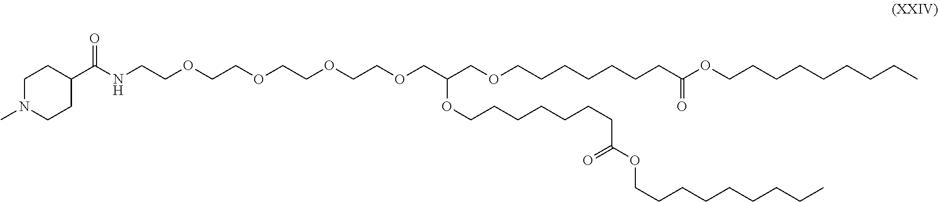 [SMILES: CCCCCCCCCOC(=O)CCCCCCCOCC(COCCOCCOCCOCCNC(=O)C1CCN(C)CC1)OCCCCCCCC(=O)OCCCCCCCCC]

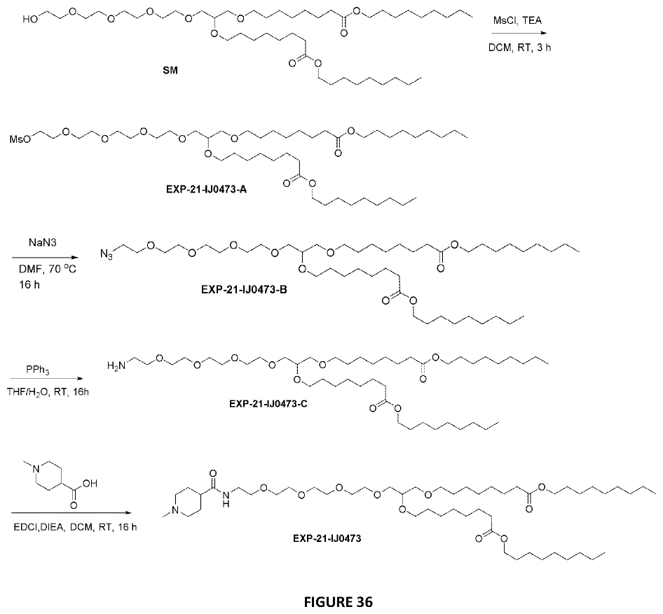

The compound XXIV is prepared according to the schema of synthesis of FIG. 36.

Refer to example 19

### Synthesis of the Intermediate LE-1-IJ0473-A

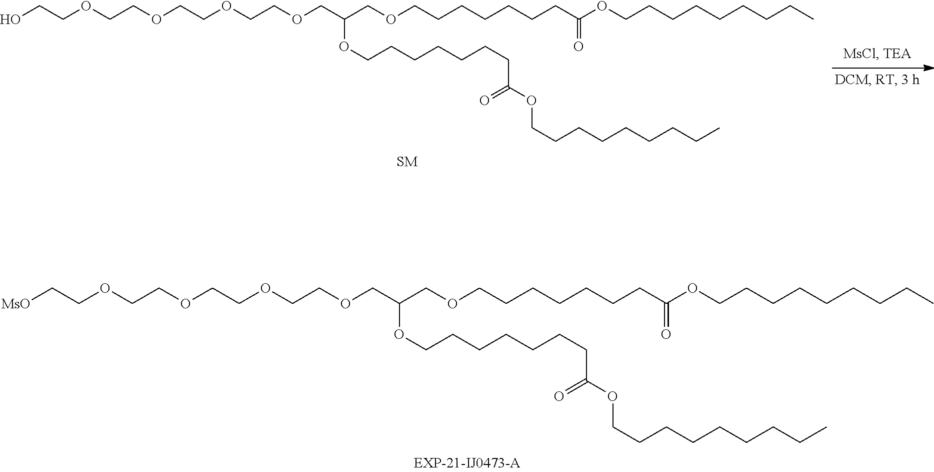 [SMILES: CCCCCCCCCOC(=O)CCCCCCCOCC(COCCOCCOCCOCCO)OCCCCCCCC(=O)OCCCCCCCCC ; "DCM, RT, 3 h" ; CCCCCCCCCOC(=O)CCCCCCCOCC(COCCOCCOCCOCCOS(C)(=O)=O)OCCCCCCCC(=O)OCCCCCCCCC]

To a mixture of nonyl 8-[3-[2-[2-[2-(2-hydroxyethoxy) ethoxy]ethoxy] ethoxy]-2-(8-nonoxy-8-oxo-octoxy) propoxy] octanoate (0.6 g, 0.745 mmol) and Triethylamine (0.126 g, 2.24 mmol) was added methanesulfonyl chloride (0.128 g, 1.12 mmol) at 0° C. The reaction mixture was stirred for 2 h at ambient temperature. The mixture was poured into DCM (100 mL) and washed with 1 N HCl, sat NaCl, dried over Na2SO4 and concentrated to give nonyl 8-[3-[2-[2-[2-(2-methylsulfonyloxyethoxy) ethoxy]ethoxy] ethoxy]-2-(8-nonoxy-8-oxo-octoxy) propoxy] octanoate (0.65 g, 99%) as colorless oil.

1H NMR (400 MHZ, CDCl3) δ 4.41-4.35 (m, 2H), 4.05 (t, J=6.7 Hz, 4H), 3.80-3.74 (m, 2H), 3.70-3.38 (m, 22H), 3.08 (s, 3H), 2.29 (t, J=7.5 Hz, 4H), 1.66-1.49 (m, 12H), 1.29 (d, J=17.1 Hz, 36H), 0.88 (t, J=6.8 Hz, 6H).

### Synthesis of the Intermediate LE-1-IJ0473-B

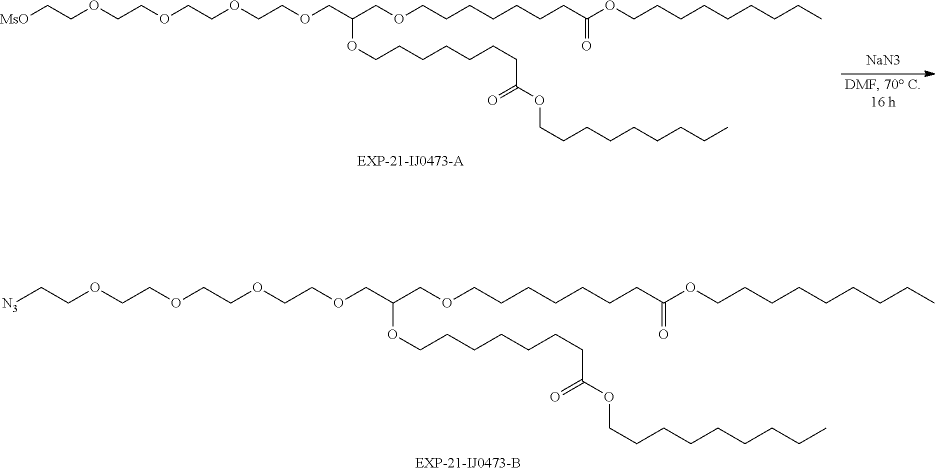 [SMILES: CCCCCCCCCOC(=O)CCCCCCCOCC(COCCOCCOCCOCCOS(C)(=O)=O)OCCCCCCCC(=O)OCCCCCCCCC ; "DMF, 70° C.16 h" ; CCCCCCCCCOC(=O)CCCCCCCOCC(COCCOCCOCCOCCN=[N+]=[N-])OCCCCCCCC(=O)OCCCCCCCCC ; "NaN3"]

A mixture of nonyl 8-[3-[2-[2-[2-(2-methylsulfonyloxyethoxy) ethoxy]ethoxy] ethoxy]-2-(8-nonoxy-8-oxo-octoxy) propoxy] octanoate (0.65 g, 0.736 mmol) and azidosodium (0.096 g, 1.47 mmol) in DMF (15 mL) at room temperature. The mixture was stirred for 16 h at 70° C. The mixture was poured into water (50 mL) and extracted with EtOAc (200 mL). The organic layer was washed with water, sat NaCl, dried over Na2SO4 and concentrated to give nonyl 8-[3-[2-[2-[2-(2-azidoethoxy) ethoxy]ethoxy] ethoxy]-2-(8-nonoxy-8-oxo-octoxy) propoxy] octanoate (0.54 g, 88%) as colorless oil.

1H NMR (400 MHZ, CDCl3) δ 4.05 (t, J=6.7 Hz, 4H), 3.72-3.60 (m, 15H), 3.59-3.36 (m, 11H), 2.28 (t, J=7.5 Hz, 4H), 1.64-1.50 (m, 12H), 1.29 (d, J=16.9 Hz, 37H), 0.88 (t, J=6.8 Hz, 6H).

### Synthesis of the Intermediate LE-1-IJ0473-C

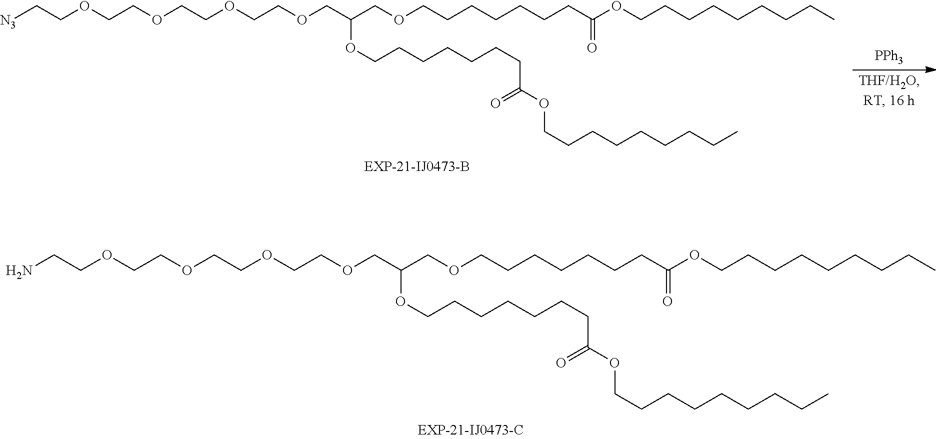 [SMILES: CCCCCCCCCOC(=O)CCCCCCCOCC(COCCOCCOCCOCCN=[N+]=[N-])OCCCCCCCC(=O)OCCCCCCCCC ; "THF/H2O,RT, 16 h" ; CCCCCCCCCOC(=O)CCCCCCCOCC(COCCOCCOCCOCCN)OCCCCCCCC(=O)OCCCCCCCCC ; "PPh3"]

A mixture of nonyl 8-[3-[2-[2-[2-(2-azidoethoxy) ethoxy]ethoxy] ethoxy]-2-(8-nonoxy-8-oxo-octoxy) propoxy] octanoate (0.54 g, 0.65 mmol) and triphenylphosphane (0.853 g, 3.25 mmol) in THF/H₂O (5 mL/1 mL) at room temperature. The reaction mixture was stirred for 16 h. The mixture was concentrated and the residue was purified by column chromatography on silica gel eluting with 2% ˜8% MeOH in DCM to afford nonyl 8-[3-[2-[2-[2-(2-aminoethoxy) ethoxy]ethoxy] ethoxy]-2-(8-nonoxy-8-oxo-octoxy) propoxy] octanoate (0.41 g, 78.4%) as colorless oil.

1H NMR (400 MHZ, CDCl3) δ 4.05 (t, J=6.8 Hz, 4H), 3.73-3.38 (m, 24H), 2.93 (t, J=5.1 Hz, 2H), 2.28 (t, J=7.5 Hz, 4H), 1.67-1.50 (m, 12H), 1.29 (d, J=16.8 Hz, 37H), 0.88 (t, J=6.8 Hz, 6H).

### Synthesis of the Compound XXIV

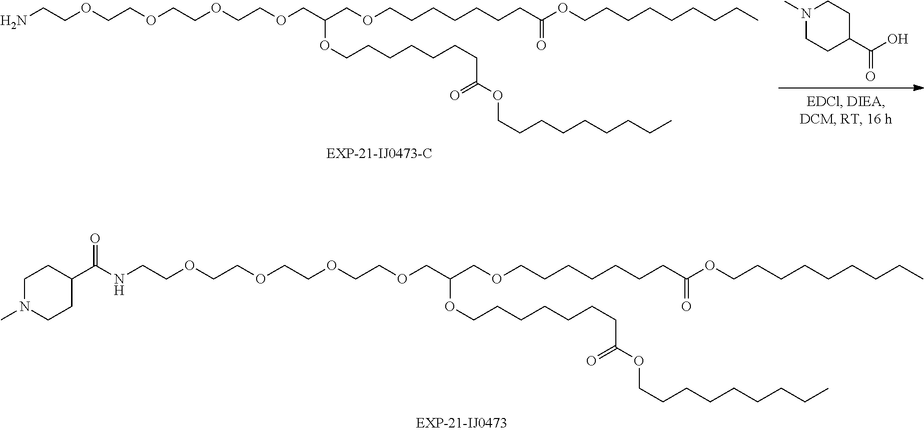

A mixture of 1-methylpiperidine-4-carboxylic acid (0.109 g, 0.765 mmol), EDC HCl (0.147 g, 0.765 mmol), in DCM (15 mL). The reaction mixture was stirred for 2 h at ambient temperature. Then added nonyl 8-[3-[2-[2-[2-(2-aminoethoxy) ethoxy]ethoxy] ethoxy]-2-(8-nonoxy-8-oxo-octoxy) propoxy] octanoate (0.41 g, 0.51 mmol) and DIEA (0.99 g, 0.765 mmol). The mixture was stirred for 16 h at room temperature. The mixture was poured into DCM (100 mL) and washed with 1 N HCl, NaCl, dried over Na2SO4 and concentrated. The residue was purified by column chromatography on silica gel eluting with 2% ˜8% MeOH in DCM to afford nonyl 8-[3-[2-[2-[2-[2-[(1-methylpiperidine-4-carbonyl) amino]ethoxy]ethoxy] ethoxy]ethoxy]-2-(8-nonoxy-8-oxo-octoxy) propoxy] octanoate (0.243 g, 51.3%) as white solid.

1H NMR (400 MHZ, CDCl3) δ 6.86 (s, 1H), 4.05 (t, J=6.7 Hz, 4H), 3.69-3.27 (m, 28H), 2.67 (s, 3H), 2.50 (s, 1H), 2.37-2.20 (m, 6H), 2.07 (d, J=14.1 Hz, 2H), 1.67-1.50 (m, 12H), 1.29 (d, J=16.7 Hz, 37H), 0.88 (t, J=6.9 Hz, 6H).

### Example 40: Synthesis of octadecan-9-yl 8-[2-(8-heptadecan-9-yloxy-8-oxooctoxy)-3-[2-[2-[2-[2-[(1-methylpiperidine-4-carbonyl) amino]ethoxy]ethoxy] ethoxy]ethoxy] propoxy] octanoate (compound XXV)

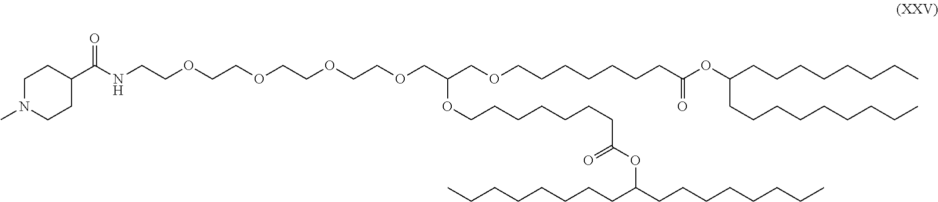 [SMILES: CCCCCCCCCC(CCCCCCCC)OC(=O)CCCCCCCOCC(COCCOCCOCCOCCNC(=O)C1CCN(C)CC1)OCCCCCCCC(=O)OC(CCCCCCCC)CCCCCCCC]

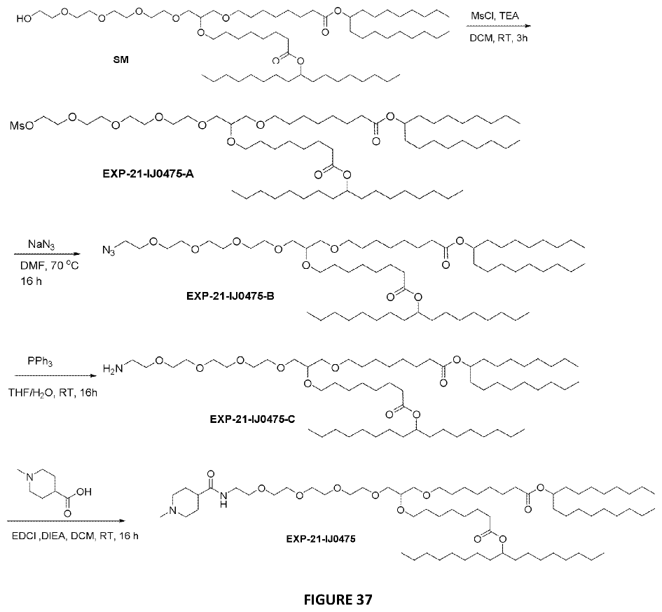

The compound XXV is prepared according to the schema of synthesis of FIG. 37.

### Synthesis of the Intermediate LE-1-IJ0475-A

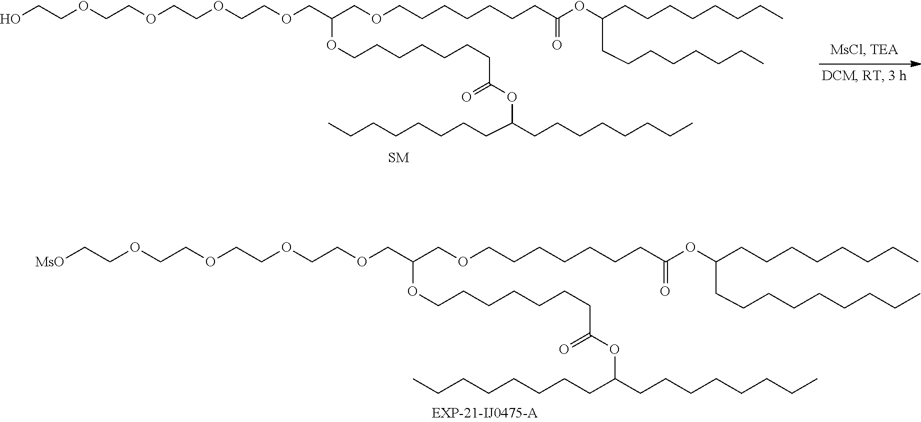 [SMILES: CCCCCCCCC(CCCCCCCC)OC(=O)CCCCCCCOCC(COCCOCCOCCOCCO)OCCCCCCCC(=O)OC(CCCCCCCC)CCCCCCCC ; "DCM, RT, 3 h" ; CCCCCCCCCC(CCCCCCCC)OC(=O)CCCCCCCOCC(COCCOCCOCCOCCOS(C)(=O)=O)OCCCCCCCC(=O)OC(CCCCCCCC)CCCCCCCC]

A mixture of 1-octyldecyl 8-[3-[2-[2-[2-(2-hydroxyethoxy) ethoxy]ethoxy] ethoxy]-2-[8-(1-octylnonoxy)-8-oxo-octoxy] propoxyloctanoate (0.97 g, 0.93 mmol) and N,N-diethylethanamine (0.282 g, 2.79 mmol) in DCM (20 mL) was added methanesulfonyl chloride (0.16 g, 1.39 mmol) at 0° C. The mixture was stirred for 3 h at ambient temperature. The mixture was poured into DCM (100 mL) and washed with 1 N HCl, sat NaCl, dried over Na2SO4, concentrated to give 1-octyldecyl8-[3-[2-[2-[2-(2-methylsulfonyloxyethoxy) ethoxy]ethoxy] ethoxy]-2-[8-(1-octylnonoxy)-8-oxo-octoxy] propoxy] octanoate (1.0 g, 96%) as colorless oil.

1H NMR (400 MHZ, CDCl3) δ 4.92-4.80 (m, 2H), 4.43-4.34 (m, 2H), 3.81-3.72 (m, 2H), 3.71-3.34 (m, 22H), 3.08 (s, 3H), 2.27 (t, J=7.1 Hz, 4H), 1.69-1.42 (m, 18H), 1.38-1.18 (m, 62H), 0.88 (t, J=6.8 Hz, 12H).

### Synthesis of the Intermediate LE-1-IJ0475-B

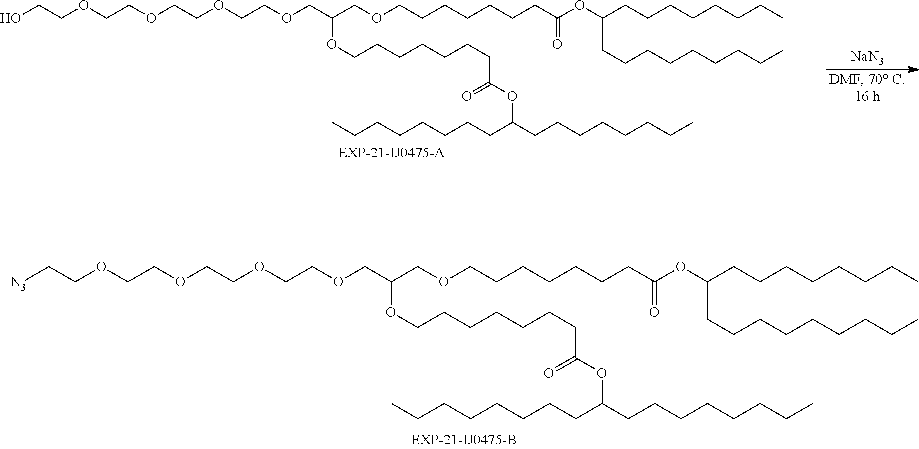 [SMILES: CCCCCCCCCC(CCCCCCCC)OC(=O)CCCCCCCOCC(COCCOCCOCCOCCO)OCCCCCCCC(=O)OC(CCCCCCCC)CCCCCCCC ; "DMF, 70° C.16 h" ; CCCCCCCCCC(CCCCCCCC)OC(=O)CCCCCCCOCC(COCCOCCOCCOCCN=[N+]=[N-])OCCCCCCCC(=O)OC(CCCCCCCC)CCCCCCCC ; "NaN3"]

A mixture of 1-octyldecyl 8-[3-[2-[2-[2-(2-methylsulfonyloxyethoxy) ethoxy]ethoxy] ethoxy]-2-[8-(1-octylnonoxy)-8-oxo-octoxy] propoxy] octanoate (1.0 g, 0.891 mmol) and azidosodium (0.116 g, 1.78 mmol) in DMF (10 mL) at room temperature. The mixture was stirred for 16 h at 70° C. The mixture was poured into water (50 mL) and extracted with EtOAc (200 mL). The organic layer was washed with water, sat NaCl, dried over Na2SO4, concentrated to give 1-octyldecyl 8-[3-[2-[2-[2-(2-azidoethoxy) ethoxy]ethoxy] ethoxy]-2-[8-(1-octylnonoxy)-8-oxo-octoxy] propoxy] octanoate (0.9 g, 94.5%) as colorless oil.

1H NMR (400 MHZ, CDCl3) δ 4.93-4.80 (m, 2H), 3.74-3.34 (m, 26H), 2.27 (t, J=7.1 Hz, 4H), 1.66-1.45 (m, 17H), 1.38-1.19 (m, 62H), 0.88 (t, J=6.8 Hz, 12H).

### Synthesis of the Intermediate LE-1-IJ0475-C

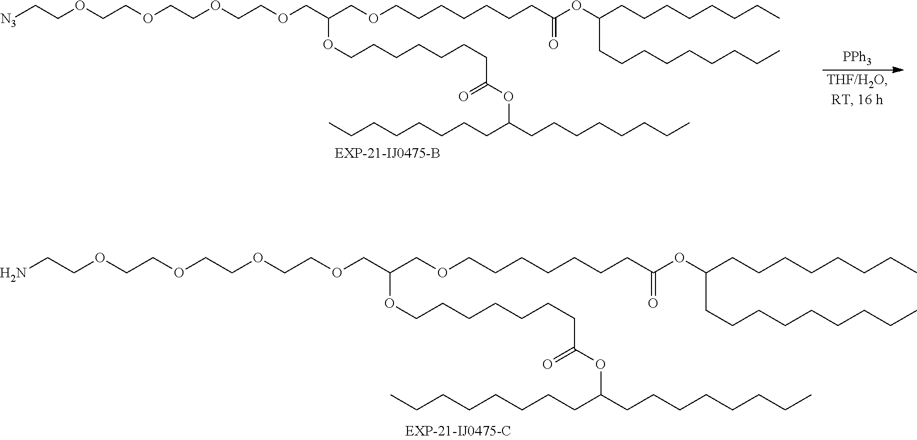 [SMILES: CCCCCCCCCC(CCCCCCCC)OC(=O)CCCCCCCOCC(COCCOCCOCCOCCN=[N+]=[N-])OCCCCCCCC(=O)OC(CCCCCCCC)CCCCCCCC ; "THF/H2O,RT, 16 h" ; CCCCCCCCCC(CCCCCCCC)OC(=O)CCCCCCCOCC(COCCOCCOCCOCCN)OCCCCCCCC(=O)OC(CCCCCCCC)CCCCCCCC ; "PPh3"]

A mixture of 1-octyldecyl 8-[3-[2-[2-[2-(2-azidoethoxy) ethoxy]ethoxy] ethoxy]-2-[8-(1-octylnonoxy)-8-oxo-octoxy] propoxy] octanoate (0.9 g, 0.842 mmol) and triphenylphosphane (1.1 g, 4.21 mmol) in THF/H₂O (10 mL/2 mL) at room temperature. The mixture was stirred for 16 h at ambient temperature. The mixture was concentrated and the residue was purified by column chromatography on silica gel eluting with 2% ˜8% MeOH in DCM to afford 1-octyldecyl 8-[3-[2-[2-[2-(2-aminoethoxy) ethoxy]ethoxy] ethoxy]-2-[8-(1-octylnonoxy)-8-oxo-octoxy] propoxy] octanoate (0.76 g, 86.6%) as colorless oil.

1H NMR (400 MHZ, CDCl3) δ 4.86 (p, J=6.3 Hz, 2H), 3.71-3.36 (m, 24H), 2.91 (t, J=5.1 Hz, 2H), 2.26 (dd, J=20.0, 12.6 Hz, 9H), 1.69-1.44 (m, 17H), 1.28 (d, J=23.4 Hz, 63H), 0.88 (t, J=6.8 Hz, 12H).

### Synthesis of the Compound XXV

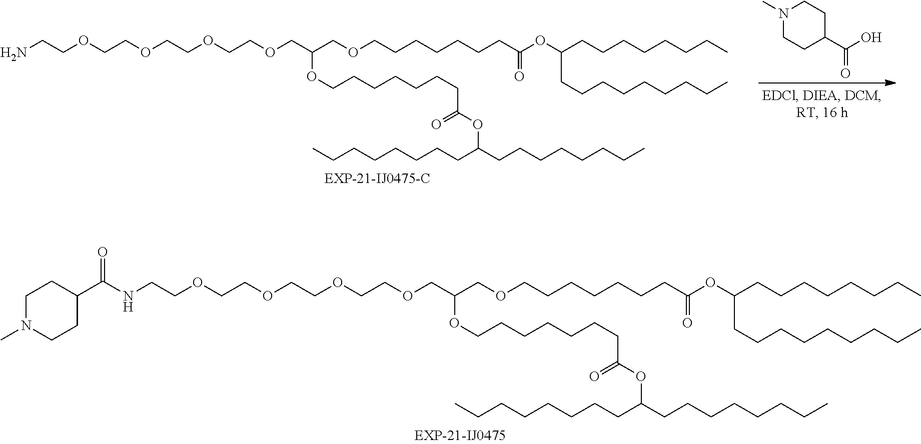

A mixture of 1-methylpiperidine-4-carboxylic acid (0.072 g, 0.504 mmol), EDC HCl (0.097 g, 0.504 mmol), in DCM (15 mL). The reaction mixture was stirred for 2 h at ambient temperature. Then added 1-octyldecyl 8-[3-[2-[2-[2-(2-aminoethoxy) ethoxy]ethoxy] ethoxy]-2-[8-(1-octylnonoxy)-8-oxo-octoxy] propoxy] octanoate (0.35 g, 0.336 mmol) and DIEA (0.065 g, 0.504 mmol). The mixture was stirred for 16 h at room temperature. The mixture was poured into DCM (100 mL) and washed with 1 N HCl, NaCl, dried over Na2SO4 and concentrated. The residue was purified by column chromatography on silica gel eluting with 2% ˜8% MeOH in DCM to afford 1-octyldecyl 8-[3-[2-[2-[2-[2-[(1-methylpiperidine-4-carbonyl) amino]ethoxy]ethoxy] ethoxy]ethoxy]-2-[8-(1-octylnonoxy)-8-oxo-octoxy] propoxyloctanoate (0.257 g, 65.6%) as white solid.

1H NMR (400 MHZ, CDCl3) δ 4.90-4.81 (m, 2H), 3.74-3.36 (m, 26H), 3.25 (s, 2H), 2.59 (s, 3H), 2.27 (t, J=7.5 Hz, 5H), 2.02 (s, 3H), 1.67-1.43 (m, 18H), 1.28 (d, J=23.2 Hz, 63H), 0.88 (t, J=6.8 Hz, 12H).

### Example 41: Synthesis of [(Z)-non-2-enyl] 8-[3-[2-[2-[2-[2-[(1-methylpiperidine-4-carbonyl) amino]ethoxy]ethoxy] ethoxy]ethoxy]-2-[8-[(Z)-non-2-enoxy]-8-oxo-octoxy] propoxy] octanoate (compound XXVI)

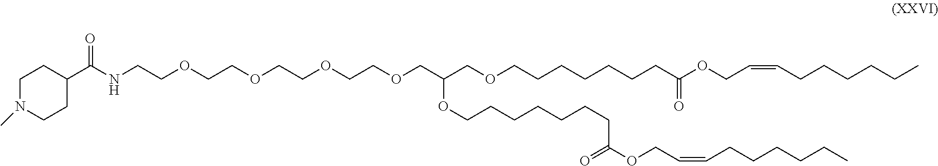 [SMILES: CCCCCC/C=C\COC(=O)CCCCCCCOCC(COCCOCCOCCOCCNC(=O)C1CCN(C)CC1)OCCCCCCCC(=O)OC/C=C\CCCCCC]

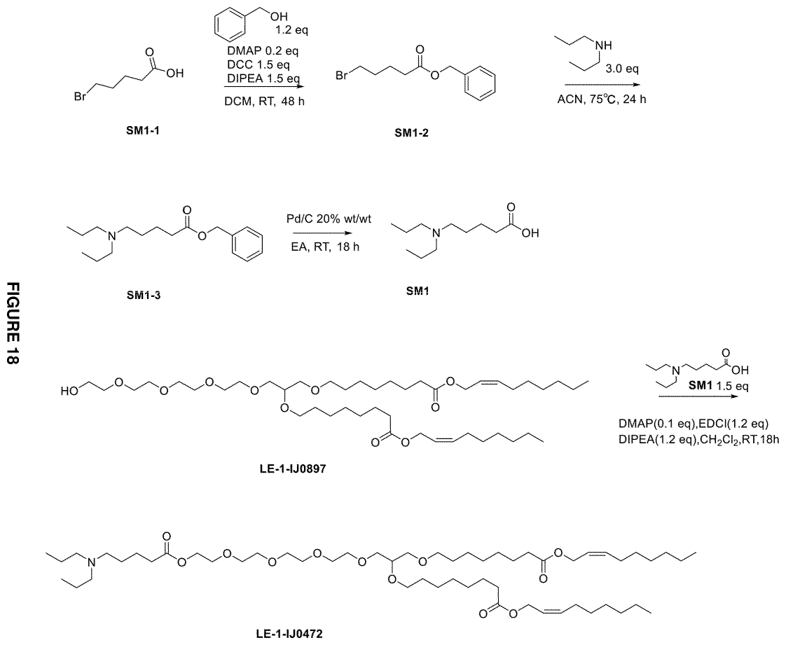

The compound L is prepared according to the schema of synthesis of FIG. 16.

Refer to example 22

### Synthesis of the Intermediate EXP-21-IJ0469-SM1

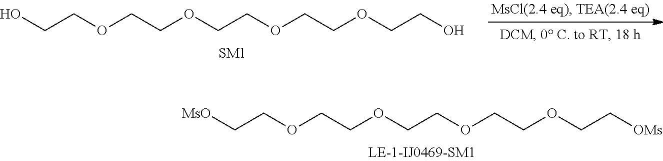 [SMILES: OCCOCCOCCOCCOCCO ; CS(=O)(=O)OCCOCCOCCOCCOCCOS(C)(=O)=O]

To a mixture of 3,6,9,12-tetraoxatetradecane-1,14-diol (100 g, 0.420 mol) and triethylamine (102 g, 1.01 mol) in DCM (600 mL) was added methanesulfonyl chloride (115 g, 1.01 mol) slowly at 0° C. The mixture was stirred overnight at room temperature. CH₂Cl₂ (400 mL) were added to the solution, and the mixture was washed with diluted HCl (1M, 1000 mL). The organic layer was washed with water (1000 mL) and brine (1000 mL), and dried over Na2SO4, filtered and concentrated to give 3,6,9,12-tetraoxatetradecane-1,14-diyl dimethanesulfonate (160 g, quant.) as a yellow oil.

1H NMR (400 MHZ, CDCl3) δ 4.45-4.33 (m, 4H), 3.82-3.74 (m, 4H), 3.72-3.57 (m, 8H), 3.08 (s, 6H).

### Synthesis of the Intermediate EXP-21-IJ0469-SM2

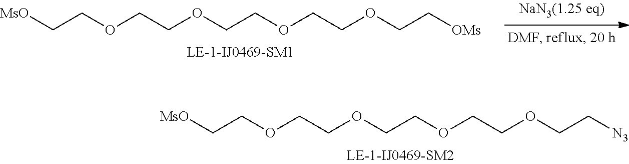 [SMILES: CS(=O)(=O)OCCOCCOCCOCCOCCOS(C)(=O)=O ; CS(=O)(=O)OCCOCCOCCOCCOCCN=[N+]=[N-]]

To a solution of 3,6,9, 12-tetraoxatetradecane-1,14-diyldimethane sulfonate (160 g, 0.406 mol) in DMF (500 mL) was added NaN₃ (33 g, 0.507 mol). The reaction mixture was stirred at 70° C. for 20 Hours. TCL showed that the starting material was disappeared, and a new spot was observed. Then water (1000 mL) was added, and the reaction mixture was extracted with ethyl acetate (1000 mL*2). The combined organic layers were washed with brine (1000 mL), dried over Na2SO4, filtered and concentrated under reduced pressure to give a residue, which was purified by column chromatography eluted with CH₃OH in DCM to give (56 g, 42.0% yield) as colorless liquid.

1H NMR (400 MHZ, CDCl3) δ 4.43-4.33 (m, 2H), 3.81-3.73 (m, 2H), 3.72-3.58 (m, 10H), 3.43-3.34 (m, 2H), 3.08 (s, 3H).

### Synthesis of the Intermediate LE-1-IJ0469-1

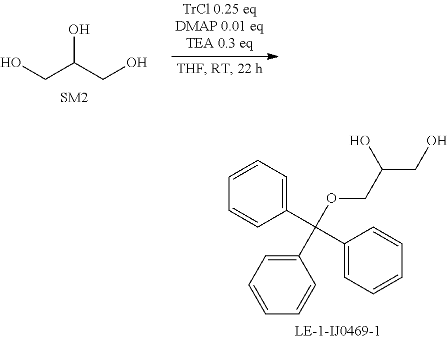 [SMILES: OCC(O)CO ; OCC(O)COC(c1ccccc1)(c1ccccc1)c1ccccc1]

Trityl chloride (75.7 g, 0.271 mol), glycerol (100 g, 1.09 mol), and N,N-dimethylpyridin-4-amine (1.33 g, 0.0109 mol) were dissolved in 500 ml of THF. After addition of triethylamine (33.0 g, 0.326 mol), the mixture was stirred for 22 h at room temperature. 300 mL of ethyl acetate and 150 mL H₂O were then added to the solution. The organic phase was collected and extracted with 2*300 mL ethyl acetate. The organic phases were combined, washed with 200 mL of 10% (w/v) NaHCO₃ and then 200 mL of brine, and dried on Na2SO4. The solvent was evaporated, and the residual oil was recrystallized in benzene/hexane. The obtained product was further purified on silica gel column (elution gradient CH₂Cl₂/MeOH) to yield 3-trityloxypropane-1,2-diol as a white solid (125 g, 34.4% yield).

### Synthesis of the Intermediate LE-1-IJ0469-2

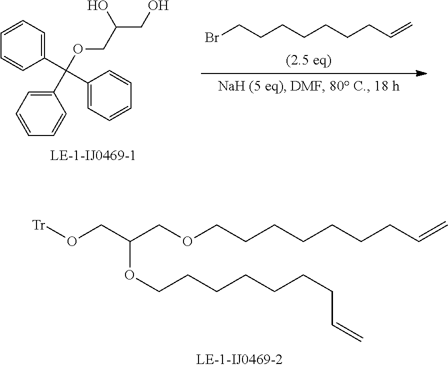

To a suspension of NaH (17.9 g, 0.748 mol) in 500 mL of anhydrous N,N-Dimethylformamide was added 3-trityloxypropane-1,2-diol (50.0 g, 0.15 mol). The mixture was heated at 80° C. for 15 minutes and cooled to room temperature. 9-bromonon-1-ene (76.7 g, 0.374 mol) in 50 mL anhydrous DMF was added dropwise to the mixture which was then heated under 80° C. for 18 h. After cooling to RT, 1000 mL of H₂O were added then extracted with ethyl acetate (500 mL*3). The combined organic layers were washed successively with 500 mL of 5% (w/v) NaHCO₃ and 500 mL of brine and dried on Na2SO4. The solvent was evaporated under reduced pressure and the residue was purified through flash chromatography column eluted with petroleum ether/ethyl acetate (0% to 20% ethyl acetate in petroleum ether) to yield a colorless oil (37.2 g, 42.7% yield).

LCMS: RT=4.67, 92%, UV (214 nm)

### Synthesis of the Intermediate LE-1-IJ0469-3

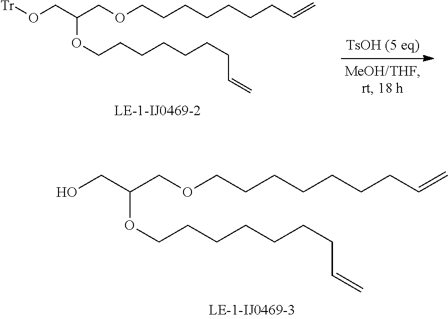

To a solution of ((2,3-bis (non-8-en-1-yloxy) propoxy) methanetriyl) tribenzene (38.3 g, 0.066 mol) in methanol/THF (600 mL, 1/1 v/v) was added 4-methylbenzenesulfonic acid (56.6 g, 0.329 mol) in one portion at room temperature and the mixture was stirred at room temperature for 18 h. TLC (4% ethyl acetate in petroleum ether) indicated that the starting material was disappeared completely, 50 mL triethylamine was added to quench the reaction and the solvent was removed under vacuum. The residue was purified by flash chromatography eluted with 20% to 30% ethyl acetate in petroleum ether (21%) to give 2,3-bis (non-8-enoxy) propan-1-ol (19.0 g, 84.9% yield) as colorless oil.

1H NMR (400 MHZ, CDCl3) δ 5.89-5.71 (m, 2H), 5.04-4.87 (m, 4H), 3.77-3.37 (m, 9H), 2.30-2.19 (m, 1H), 2.12-1.95 (m, 5H), 1.62-1.51 (m, 4H), 1.48-1.20 (m, 18H).

### Synthesis of the Intermediate LE-1-IJ0469-4

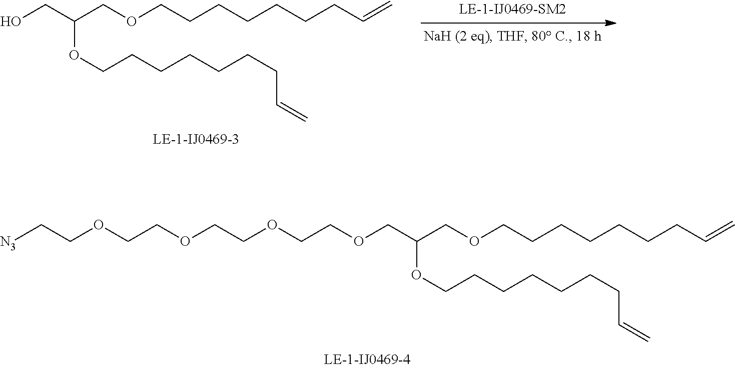 [SMILES: C=CCCCCCCCOCC(CO)OCCCCCCCC=C ; C=CCCCCCCCOCC(COCCOCCOCCOCCN=[N+]=[N-])OCCCCCCCC=C]

To a mixture of 2,3-bis (non-8-enoxy) propan-1-ol (5.0 g, 1.0 eq) added NaH (0.71 g, 2.0 eq) in 200 mL dry THF then stirred 15 min at 80° C. Then after the solvent was restore room temperature added 2-[2-[2-(2-azidoethoxy) ethoxy]ethoxy]ethyl methanesulfonate (5.24 g, 1.2 eq) dissolved in 60 mL of dry THF. The reaction mixture was stirred reflux (80° C.) overnight. The reaction mixture was cooled to rt, and water (200 mL) was added. EtOAc (400 mL) was added, the mixture was shaken, the layers were separated, and the organic layer was collected. The aqueous layer was extracted with EtOAc (400 mL*2). The combined organic layers were washed with brine and dried over Na₂SO₄. The residue was purified by flash column chromatography on silica gel eluting with EA in PE (0-15%) (14%) to give target product (5.2 g, 65.4% yield) as yellow oil.

1H NMR (400 MHZ, CDCl₃) δ 5.88-5.73 (m, 2H), 5.03-4.90 (m, 4H), 3.71-3.60 (m, 16H), 3.60-3.36 (m, 12H), 2.08-2.00 (m, 4H), 1.61-1.49 (m, 4H), 1.41-1.26 (m, 16H).

### Synthesis of the Intermediate LE-1-IJ0469-5

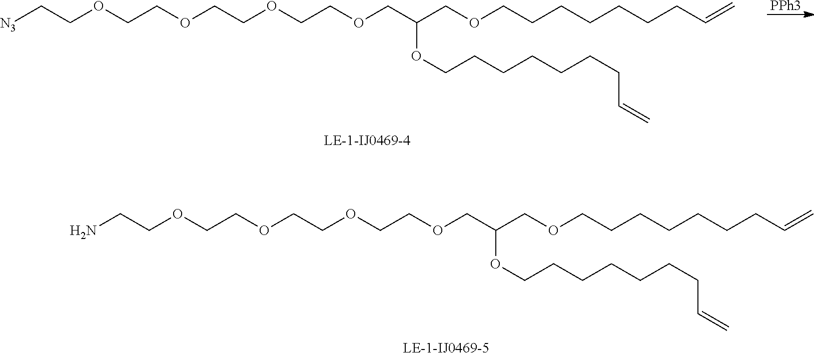 [SMILES: C=CCCCCCCCOCC(COCCOCCOCCOCCN=[N+]=[N-])OCCCCCCCC=C ; C=CCCCCCCCOCC(COCCOCCOCCOCCN)OCCCCCCCC=C ; "PPh3"]

9-[3-[2-[2-[2-(2-azidoethoxy) ethoxy]ethoxy] ethoxy]-2-non-8-enoxy-propoxy] non-1-ene (5.2 g, 9.6 mmol eq) and triphenylphosphine (7.55 g, 28.8 mmol) were dissolved in THF (150 mL) and water (15 mL). The reaction was stirred overnight to room temperature. The reaction was concentrated and purified by flash column chromatography on silica gel eluting with 5% to 25% MeOH in DCM to give 2-[2-[2-[2-[2,3-bis (non-8-enoxy) propoxy] ethoxy]ethoxy] ethoxylethanamine (3.6 g, 72.7% yield) as colorless oil.

¹H NMR (400 MHZ, CDCl3) δ 5.87-5.74 (m, 2H), 5.03-4.90 (m, 4H), 3.71-3.60 (m, 13H), 3.61-3.38 (m, 12H), 2.87 (t, J=5.2 Hz, 2H), 2.04 (q, J=6.7 Hz, 4H), 1.82 (s, 2H), 1.60-1.50 (m, 4H), 1.41-1.27 (m, 16H).

### Synthesis of the Intermediate LE-1-IJ0469-6

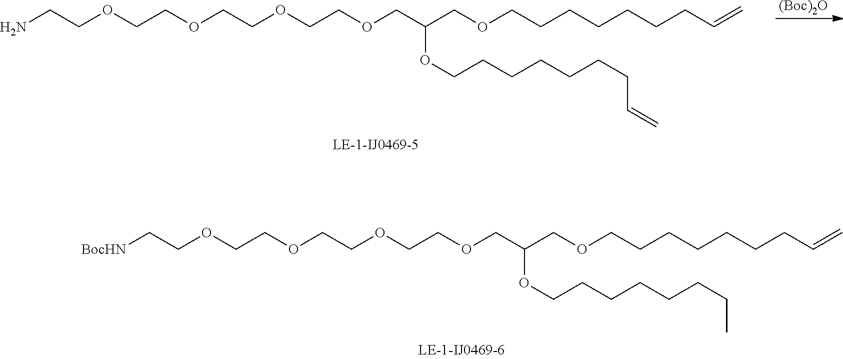 [SMILES: "(Boc)2O" ; C=CCCCCCCCOCC(COCCOCCOCCOCCN)OCCCCCCCC=C ; C=CCCCCCCCOCC(COCCOCCOCCOCCNC(=O)OC(C)(C)C)OCCCCCCCC]

To a solution of 2-[2-[2-[2-[2,3-bis (non-8-g, 6.98 mmol) and N,N-enoxy) propoxy] ethoxy]ethoxy] ethoxy] ethanamine (3.6 diethylethanamine (1.41 g, 14 mmol) in 100 mL of CH₂Cl₂ at room temperature was added tert-butoxycarbonyl tert-butyl carbonate (1.83 g, 8.38 mmol) and the mixture was stirred overnight at room temperature. The mixture was quenched with HCl (20 mL 0.5 mol) and the organic layer was separated and washed with H₂O and brine. The organic phase was dried over Na2SO4, filtered and concentrated. The residue was purified by flash chromatography (silica gel, EA: PE-2:3) to obtain tert-butyl N-[2-[2-[2-[2-[2,3-bis (non-8-enoxy) propoxy] ethoxy]ethoxy] ethoxy]ethyl] carbamate (3.7 g, 86.1% yield) as colorless oil.

¹H NMR (400 MHZ, CDCl3) δ 5.87-5.74 (m, 2H), 5.02-4.91 (m, 4H), 3.70-3.58 (m, 13H), 3.59-3.39 (m, 12H), 3.35-3.27 (m, 2H), 2.04 (q, J=6.7 Hz, 4H), 1.74 (s, 1H), 1.61-1.50 (m, 4H), 1.44 (s, 9H), 1.40-1.24 (m, 16H).

### Synthesis of the Intermediate LE-1-IJ0469-7

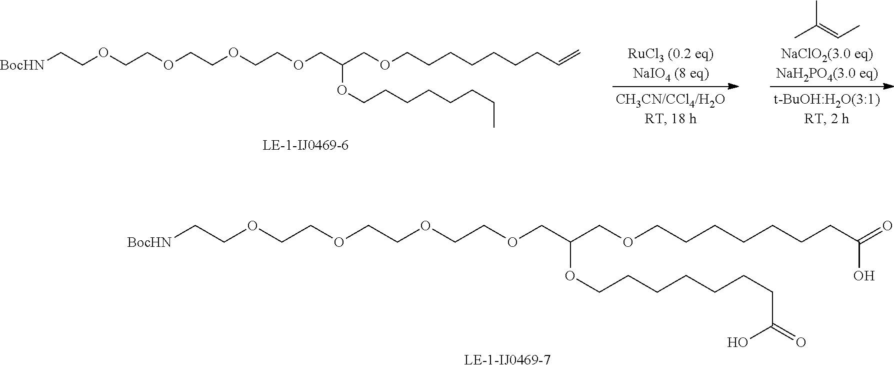

To a solution of tert-butyl N-[2-[2-[2-[2-[2,3-bis (non-8-enoxy) propoxy] ethoxy]ethoxy] ethoxy]ethyl] carbamate (12.9 g, 20.9 mmol) in MeCN (200 mL), CCl₄ (200 mL) and water (200 mL) was added NalO₄ (52.2 g, 244 mmol) and RuCl₃ (1.27 g, 6.1 mmol). The reaction mixture was stirred at room temperature for 18 h. The reaction was filtered, and the filtrate was diluted with ethyl acetate (800 mL) and washed with 1N aq. HCl (900 mL). The organic layer was washed with Na2S₂O₃ solution (700 mL*2) and then dried over sodium sulfate, filtered and concentrated to give 8-[3-[2-[2-[2-[2-(tert-butoxycarbonylamino) ethoxy]ethoxy] ethoxy]ethoxy]-2-(7-carboxyheptoxy) propoxyloctanoic acid (13.5 g, quant.) as yellow oil which was used without further purification. N-[2-[2-[2-[2-[2,3-bis (non-8-enoxy) propoxy] ethoxy]ethoxy] ethoxy]ethyl] carbamate (12.9 g, 20.9 mmol) was dissolved in t-BuOH: H₂O (3:1, 200 mL), containing NaH₂PO₄ (4.61 g, 17.0 mmol), 2-methy-2-butene (40 mL) and sodium chlorite (4.61 g, 51.0 mmol). The reaction was stirred for 2 h at room temperature and LCMS indicated that the starting material was consumed. The reaction mixture was diluted with H₂O. The aqueous layer was extracted with ethyl acetate (800 mL*2). The residue was purified by flash column chromatography on silica gel eluting with CH₃OH in DCM (0-15%) 9% to give target product (12.0 g, 87.9% yield) as light-yellow oil.

1H NMR (400 MHZ, CDCl3) δ 5.11 (d, J=27.7 Hz, 1H), 3.76-3.23 (m, 28H), 2.33 (t, J=7.3 Hz, 4H), 1.67-1.51 (m, 8H), 1.44 (s, 10H), 1.35 (d, J=10.3 Hz, 12H).

### Synthesis of the Intermediate LE-1-IJ0469-8

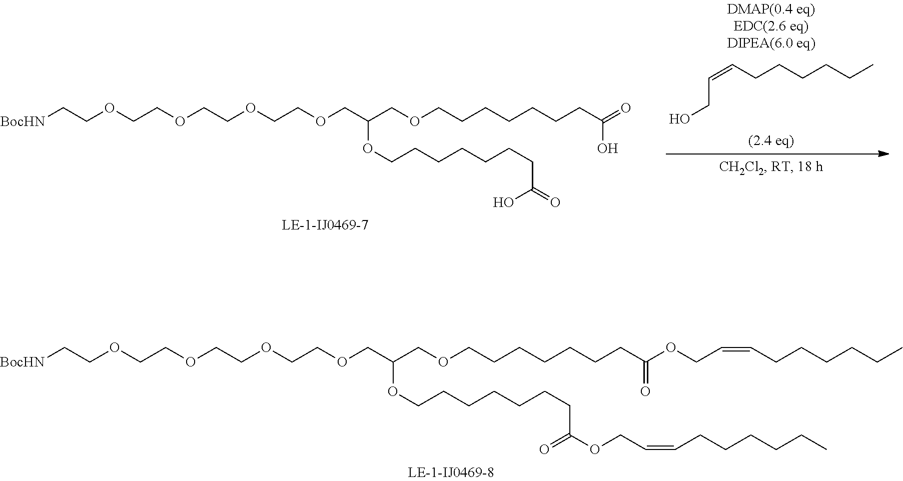

8-[3-[2-[2-[2-[2-(tert-butoxycarbonylamino) ethoxy]ethoxy] ethoxy]ethoxy]-2-(7-carboxyheptoxy) propoxy] octanoic acid (1.0 g, 1.53 mmol) and (Z)-non-2-en-1-ol (0.524 g, 3.68 mmol) in dry dichloromethane (20 mL) under nitrogen were added N,N-Diisopropylethylamine (1.19 g, 9.2 mmol), DMAP (0.075 g, 0.614 mmol) and EDCl (0.761 g, 3.99 mmol). The mixture was stirred at room temperature for 18 h. The reaction was diluted with dichloromethane and washed with brine. The organic layer was dried over sodium sulfate, filtered and concentrated. The residue was purified by flash chromatography eluted with 20% to 55% (27%) ethyl acetate in petroleum ether to give [(Z)-non-2-enyl] 8-[3-[2-[2-[2-[2-(tert-butoxycarbonylamino) ethoxy]ethoxy] ethoxy]ethoxy]-2-[8-[(Z)-non-2-enoxy]-8-oxo-octoxy] propoxy] octanoate (0.85 g, 61.5% yield) as light yellow oil.

1H NMR (400 MHZ, CDCl3) 0 5.64 (dt, J=10.8, 7.5 Hz, 2H), 5.52 (dt, J=11.0, 6.8 Hz, 2H), 5.06 (s, 1H), 4.62 (d, J=6.8 Hz, 4H), 3.68-3.39 (m, 27H), 3.31 (d, J=5.2 Hz, 2H), 2.30 (t, J=7.5 Hz, 4H), 2.14-2.04 (m, 4H), 1.65-1.59 (m, 4H), 1.58-1.51 (m, 4H), 1.44 (s, 10H), 1.40-1.24 (m, 30H), 0.88 (t, J=6.9 Hz, 6H).

### Synthesis of the Intermediate LE-1-IJ0469-9

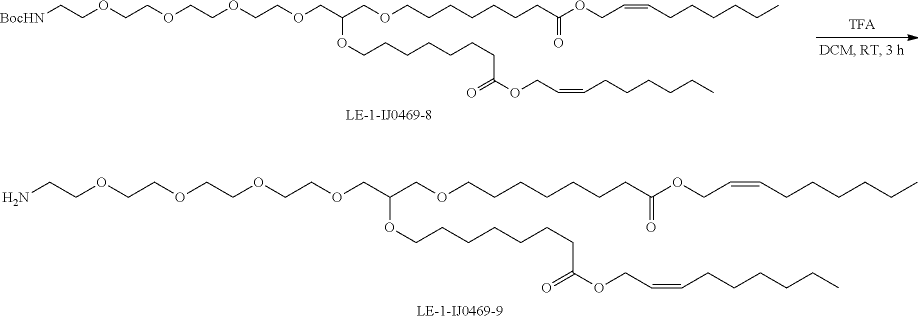 [SMILES: CCCCCC/C=C\COC(=O)CCCCCCCOCC(COCCOCCOCCOCCNC(=O)OC(C)(C)C)OCCCCCCCC(=O)OC/C=C\CCCCCC ; CCCCCC/C=C\COC(=O)CCCCCCCOCC(COCCOCCOCCOCCN)OCCCCCCCC(=O)OC/C=C\CCCCCC]

[(Z)-non-2-enyl] 8-[3-[2-[2-[2-[2-(tert-butoxycarbonylamino) ethoxy]ethoxy] ethoxy]ethoxy]-2-[8-[(Z)-non-2-enoxy]-8-oxo-octoxy] propoxy] octanoate (500 mg, 0.56 mmol) in DCM (8 mL) cooled to 0° C. was added TFA (2 mL) and the mixture was stirred at room temperature for 3 h. TLC indicated that the starting material was disappeared. The reaction was quenched with a saturated NaHCO₃at 0° C. The organic layer was washed with a saturated NaHCO₃, 0.1 M NaOH and brine, dried over sodium sulfate. The solvent was remove under vacuum to give [(Z)-non-2-enyl] 8-[3-[2-[2-[2-(2-aminoethoxy) ethoxy]ethoxy] ethoxy]-2-[8-[(Z)-non-2-enoxy]-8-oxo-octoxy] propoxy] octanoate (370 mg, 83.3% yield).

1H NMR (500 MHZ, CDCl3) δ 5.68-5.60 (m, 2H), 5.51 (dt, J=19.6, 7.7 Hz, 2H), 4.62 (d, J=6.8 Hz, 4H), 3.70-3.37 (m, 25H), 2.87 (t, J=5.2 Hz, 2H), 2.30 (t, J=7.6 Hz, 4H), 2.10 (q, J=7.2 Hz, 4H), 1.58 (dd, J=35.2, 6.3 Hz, 9H), 1.40-1.23 (m, 33H), 0.88 (t, J=6.9 Hz, 6H).

### Synthesis of the Compound XXVI

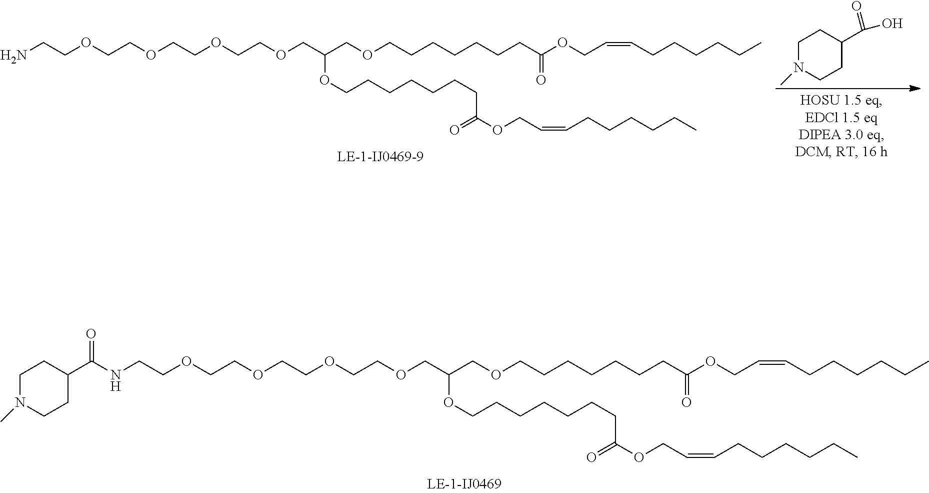

A mixture of 1-methylpiperidine-4-carboxylic acid (0.132 g, 0.925 mmol), EDC HCl (0.133 g, 0.694 mmol), N-Hydroxysuccinimide (0.080 g, 0.694 mmol) in DCM (15 mL). The reaction mixture was stirred for 2 h at rt. Then [(Z)-non-2-enyl] 8-[3-[2-[2-[2-(2-aminoethoxy) ethoxy]ethoxy] ethoxy]-2-[8-[(Z)-non-2-enoxy]-8-oxo-octoxy] propoxy] octanoate (0.37 g, 0.462 mmol) and DIPEA (0.179 g, 1.39 mmol) were added. The mixture was stirred for 16 h at rt. The mixture was poured into DCM (100 mL) and washed with NaHCO₃ solution and brine, dried over Na2SO4 and concentrated. The residue was purified by column chromatography on silica gel eluting with MeOH in DCM (13%) to afford [(Z)-non-2-enyl] 8-[3-[2-[2-[2-[2-[(1-methylpiperidine-4-carbonyl) amino]ethoxy]ethoxy] ethoxy] ethoxy]-2-[8-[(Z)-non-2-enoxy]-8-oxo-octoxy] propoxy] octanoate (278 mg, 65% yield).

1H NMR (400 MHZ, CDCl3) δ 5.63 (t, J=9.1 Hz, 2H), 5.56-5.48 (m, 2H), 4.62 (d, J=6.7 Hz, 4H), 3.68-3.28 (m, 23H), 2.69 (s, 4H), 2.30 (t, J=7.3 Hz, 4H), 2.23 (d, J=7.2 Hz, 2H), 2.10 (dd, J=14.5, 7.3 Hz, 5H), 1.86 (s, 7H), 1.58 (d, J=29.6 Hz, 9H), 1.40-1.19 (m, 29H), 0.88 (t, J=6.8 Hz, 6H).

### Example 42: Manufacture of LNPs Containing a Nucleic Acid/mRNA

### Preparation of the Organic Phase

Lipids were dissolved in ethanol at molar ratios of 50:10:38.5:1.5 (lipidic compound: DSPC: cholesterol: DMPG-PEG).

The lipidic compound of formula (VI), (VII), (VIII), (XI), or (XII) were used to manufacture the LNPs.

Cholesterol was obtained from Sigma Aldrich (C₃₀₄₅). DSPC (1,2-distearoyl-sn-glycero-3-phosphocholine and DMPG-PEG 2000 were obtained from Avanti Polar Lipids.

3.8 mg of DSPC, 2 mg of DMPG-PEG2000 and 7.2 mg of cholesterol were dissolved in 1477 μL of ethanol. Then 210 μL of lipidic compound of formula (VI), (VII), (VIII), (XI), (XII) stock solution (100 mg/mL in ethanol) was added to obtain 20 mg/ml of lipid phase solution.

### Preparation of the Aqueous Phase

Nucleic acid/mRNA concentration to be used in aqueous phase was calculated to obtain a cationic amino group/anionic phosphate group ratio of 6 (N/P=6). This concentration was determined from the lipidic compound concentration assuming 1 μg mRNA corresponds to 0.003 μmol of phosphate. A CleanCap® EPO mRNA (5moU), non-replicative, highly purified, mRNA encoding the human erythropoietin obtained from TriLink Biotechnologies, San Diego, CA (catalogue number L7209; hEPO mRNA) was used.

Since 1.5 mL of aqueous solution is needed to make 2 mL of LNPs when using a ratio of aqueous solution to ethanol solution of 3:1 with the NanoAssembIR® (Nanoassemblr Benchtop from Precision Nanosystem; Belliveau et al., Molecular Therapy-Nucleic Acids (2012)), the required mRNA concentration was calculated to be 305 μg/mL.

The mRNA solution was prepared in 50 mM citrate buffer pH 4.0.

### LNPs Preparation

LNPs were prepared using a NanoAssembIR equipment according to manufacturer recommendations.

The aqueous and organic phases were each loaded in a syringe suitable for NanoAssembIR according to manufacturer recommendations. The flow rate was set up at a ratio: 3:1 and total flow rate: 4 ml/min. The aqueous and lipid phases were then mixed to obtain the LNPs.

### LNPs Purification and Harvest

The obtained LNPs were dialyzed against a citrate buffer (50 mM-pH 4.0) to remove residual ethanol and then twice against a PBS buffer (pH 7.4). Each dialysis was carried out at least during 12 hours at 4° C. The LNPs were then filtered through a 0.22 μm filter and stored under nitrogen at ±4° C.

### Example 43: In Vitro LNP Cell Delivering of Luciferase mRNA

### Materials and Methods

### LNPs Reagents

1,2-distearoyl-sn-glycero-3-phosphocholine (DSPC) and DSPC-PEG were purchased from Avanti Polar Lipids (Alabaster, Albany). 3B-Hydroxy-5-cholestene, 5-Cholesten-3B-ol (Cholesterol) and Acrodisc Syringe Filters with Supor Membrane, Sterile-0.2 μm, 13 mm, were purchased Sigma Aldrich (St. Louis, Missouri). COATSOME@ SS-OP (SS-OP) was purchased from NOF America Corporation (White Plains, NY headquartered in Japan). Nuclease-free water, 10× PBS Buffer pH 7.4, Sodium Citrate, Dihydrate, Citric Acid, Sodium Chloride, Sucrose, Ethyl Alcohol, BD Vacutainer General Use Syringe Needles (BD Blunt Fill Needle 18G), BD Slip Tip Sterile Syringes (3 ml & 1 mL), Amicon Ultra Centifugal Filter Units, DNA-free microcentrifuge tubes (1.5 mL), Invitrogen RNase-free Microfuge Tubes (0.5 mL), Invitrogen Conical Tubes (15 mL) (DNase-RNase-free), Fisher Brand Semi-Micro Cuvette, Quant-iT™ RiboGreen® RNA Assay Kit and RnaseZap™ were purchased from Thermo Fisher Scientific (Waltham, Massachusetts).

mRNA encoding luciferase was purchased from TriLink™ Biotechnologies (mRNA-Luc-Ref.: L-7602).

### LNPs Manufacturing

LNPs comprising 55-240 ng of mRNA encoding a luciferase (CleanCap® FLuc mRNA (TriLink L-7202)-luc mRNA) were prepared with lipidic compound of formula (VII) or COATSOME@ SS-OP (SS-OP) according to the protocol of Example 42.

The LNPs formulations was: ionizable lipid/Cholesterol/DSPC/DSPC-PEG at 50/39.5/10/0.5 (LNPs-1) or 40/44.5/15/0.5 molar ratios (LNPs-2).

LNPs Lip (VII)-1 were prepared with 20 mM of ionizable cationic lipid Lip (VII) and mRNA at 44 μg/ml in a citrate buffer 50 mM (9.6 μl luc mRNA±26.4 μl citrate 50 mM) to obtain a ratio N/P=6. The molar ratio of the obtained LNPs was: Lipid (VII)/cholesterol/DSPC/DSPC-PEG2000 at 50/39.5/10/0.5.

LNPs Lip (VII)-2 were prepared with 20 mM of ionizable cationic lipid Lip (VII) and mRNA at 36 μg/ml in a citrate buffer 50 mM (7.7 luc mRNA±28.3 μl citrate 50 mM) to obtain a ratio N/P=6. The molar ratio of the obtained LNPs was: Lipid (VII)/cholesterol/DSPC/DSPC-PEG2000 at 40/44.5/15/0.5.

LNPs SS-OP-1 were prepared with 20 mM of ionizable cationic lipid SS-OP and mRNA at 44 g/ml in a citrate buffer 50 mM (9.6 μl luc mRNA±26.4 μl citrate 50 mM) to obtain a ratio N/P=12. The molar ratio of the obtained LNPs was: SS-OP/cholesterol/DSPC/DSPC-PEG2000 at 50/39.5/10/0.5.

LNPs SS-OP-2 were prepared with 20 mM of ionizable cationic lipid SS-OP and mRNA at 36 g/ml in a citrate buffer 50 mM (7.7 luc mRNA±28.3 μl citrate 50 mM) to obtain a ratio N/P=12. The molar ratio of the obtained LNPs was: SS-OP/cholesterol/DSPC/DSPC-PEG2000 at 40/44.5/15/0.5.

The prepared LNPs had the following formulations and characteristics:

TABLE 3A
LNPs formulations & characteristic

| LNPs | Lipids | Lipid ratio | N/P |
|---|---|---|---|
| LNP Lip. (VII) -1 | Lip. (VII)/Chol/DSPC/ | 50/39.5/10/.05 | 6 |
| | DSPC-PEG | | |
| LNP Lip. (VII) -2 | Lip. (VII)/Chol/DSPC/ | 40/44/15/0.5 | 6 |
| | DSPC-PEG | | |
| LNP SSOP -1 | SSOP/Chol/DSPC/DSPC-PEG | 50/39.5/10/.05 | 12 |
| LNP SSOP -2 | SSOP/Chol/DSPC/DSPC-PEG | 40/44/15/0.5 | 12 |

TABLE 3B
LNPs formulations & characteristic

| | Size LNP | Polydispersity Index | % |
|---|---|---|---|
| LNPs | (nm) | (PDI) | encapsulation |
| LNP Lip. (VII) -1 | 88 | 0.17 | 82 |
| LNP Lip. (VII) -2 | 89 | 0.14 | 80 |
| LNP SSOP -1 | 93 | 0.16 | 100 |
| LNP SSOP -2 | 87 | 0.23 | 100 |

The size, encapsulation rate and polydispersity index (PDI) were measured as follows.

### RNA Titration/Encapsulation Rate

The percentage of encapsulated mRNA and concentration of mRNA in LNPs were measured using the Quant-iT Ribogreen RNA reagent kit according to manufacturer recommendations (Invitrogen Detection Technologies) and quantified with a fluorescent microplate reader or a standard spectrophotometer using fluorescein excitation and emission wavelength.

For quantification of non-encapsulated RNA, LNPs were diluted in Tris/EDTA assay buffer (10 mM Tris-HCl, 1 mM EDTA, pH 7.5). For quantification of total amount of RNA, LNPs were diluted in Tris/EDTA assay buffer (10 mM Tris-HCl, 1 mM EDTA, pH 7.5) containing 0.5% (v/v) Triton X100. Ribogreen dye (200× diluted) was added to the samples (50/50 mix; sample/Ribogreen reagent), mixed thoroughly and incubated 5 min at room temperature in the dark.

Fluorescence was measured on the plate reader.

### Lipid Quantification

It was assumed that there was no loss of lipids during the formulation process. The total lipid concentration before dialysis was then assumed to be 5 mg/mL. The final lipid concentration was defined by taking into account the dilution factor occurring during the dialysis step.

### Particle Size Distribution, Polydispersity Index Zeta Potential, and Osmolarity

Zeta potential and particle size distribution of LNPs were measured by using a zeta sizer Nano ZS light scattering instrument (Malvern Instruments) according to manufacturer recommendations.

Particle sizes were reported as the Z-average size (harmonic intensity averaged particle diameter) along with the Polydispersity Index (PDI), an indicator of the “broadness” of the particle size distribution. Samples were diluted to 1/100 in phosphate buffered saline (PBS) before measurement. For accurate particle sizing with the Nano ZS, the viscosity of the buffer and the refractive index of the material had to be provided to the equipment software (PBS: v=1.02 cP, RI=1.45).

For zeta potential measurements, samples were diluted in water (v=0.8872 cP). Data were analyzed using the Zetasizer Software V 7.11 from Malvern

The osmolarity and the pH of formulations were measured by using respectively a micro-sample Osmometer (Fiske Associates model) and a pHmeter (Mettler Toledo) according to the equipment manufacturer instructions.”

Cell Culture and mRNA Delivery

An assay procedure has been optimized to test the transfection capability of LNP formulated with luciferase mRNA. This assay uses the monolayer hepatic cell line Huh7. One day prior to transfection, 1.0×10⁴ cells per well were seeded in 20 μL of complete cell culture media into black clear-bottom 384-well plates (Greiner) and placed into an incubator (37° C. and 5% CO₂).

The following day, 5 μl of LNPs containing 55-240 ng of Luc-mRNA was added into cells using the acoustic droplet dispenser ECHO 525 (Labcyte).

The positive control is obtained by mixing of Lipofectamine MessengerMax™ (Invitrogen LMRNA001) with CleanCap® FLuc mRNA (TriLink L-7202). After incubation for 1H at room temperature, 5 μl of this mix of transfection is added into cells using Cybiwell dispenser (Cybio) corresponding to 55 ng mRNA per well.

The negative controls were obtained by addition of CleanCap® FLuc mRNA (TriLink L-7202) to cells plate using Cybiwell dispenser (Cybio) corresponding to 55 ng mRNA per well or were the cells alone.

Several assay plates are prepared similarly (for transfection and cytotoxicity readout) and incubated for 24H at 37° C. with 5% CO2.

At the end of the incubation time, the luciferase production is measured using Bright-Glo™ Luciferase Assay System (Promega). The cytotoxicity readout is performed by cellular ATP detection using CellTiter-Glo@ Luminescent Cell Viability Assay (Promega). The signal is both measured using a Pherastar plate reader (BMG Labtech).

### Results

The obtained results are summarized in the Table 4:

TABLE 4A
Luminescence and luciferase expression

| | | OptiMEM | |
|---|---|---|---|
| | concentration of | | % |
| LNPs | mRNA/wells (ng/wells) | RLU | transfection |
| LNP Lip. (VII) -1 | 220 | 98 472 | 105 |
| LNP Lip. (VII) -2 | 180 | 120 081 | 128 |
| LNP SSOP -1 | 240 | 557 | 0 |
| LNP SSOP -2 | 200 | 1 363 | 1 |
| Controls | POS: 50 nl | 93 552 | 100 |
| | Lipofectamine + | | |
| | 55 ng mRNA | | |
| | NEG: 55 ng luc mRNA | 320 | 0 |
| | NEG: cells alone | 154 | 0 |

POS: positive control
NEG: negative control

TABLE 4B
Luminescence and luciferase expression

| | | OptiMEM + 10% serum | |
|---|---|---|---|
| | concentration of | | % |
| LNPs | mRNA/wells (ng/wells) | RLU | transfection |
| LNP Lip. (VII) -1 | 220 | 97 629 | 362 |
| LNP Lip. (VII) -2 | 180 | 11 5774 | 429 |
| LNP SSOP -1 | 240 | 227 | 1 |
| LNP SSOP -2 | 200 | 522 | 2 |
| Controls | POS: 50 nl | 27 050 | 100 |
| | Lipofectamine + | | |
| | 55 ng mRNA | | |
| | NEG: 55 ng luc mRNA | 212 | 0 |
| | NEG: cells alone | 78 | 0 |

POS: positive control
NEG: negative control

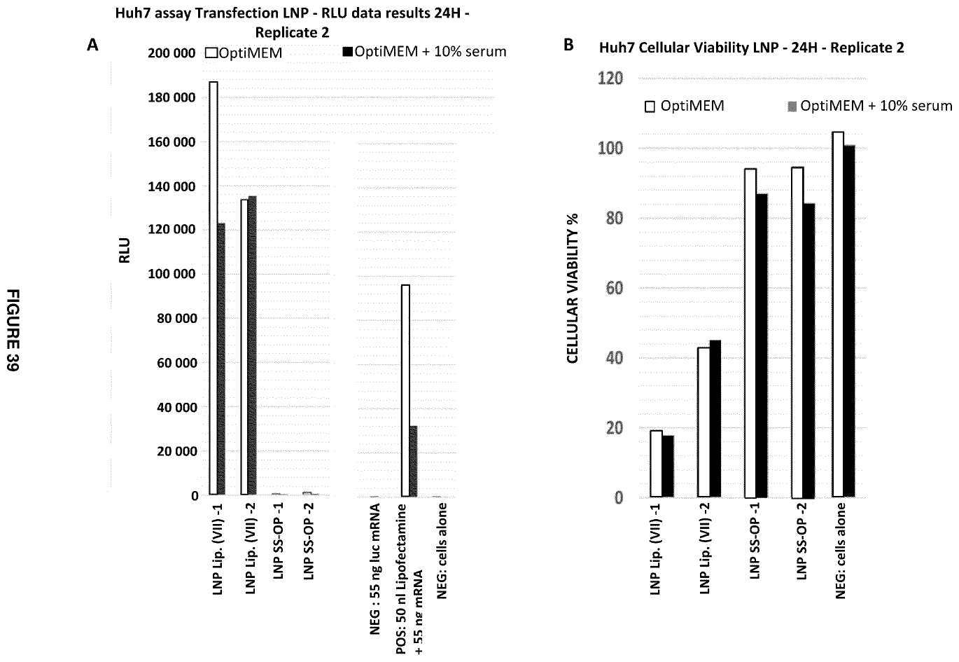

As shown by the results presented in Table 4 (A and B) or on FIG. 39A, the LNPs prepared with a lipidic compound of the disclosure (Lip. (VII)) and containing mRNA encoding the Firefly luciferase were able to increase the delivery of the mRNA and the expression of the luciferase. The luminescence and transfection rate obtained with the LNPs of the disclosure were far above the luminescence and transfection rate obtained with the benchmark LNPs SS-OP.

Further, as shown on FIG. 39B, the viability of the cells was weakly affected by the LNPs.

### Example 44: LNP Delivered Luciferase mRNA Imaging

The purpose of the study was to determine the liver transduction efficiency and tissue biodistribution of different LNP formulations in normal mice.

### Materials and Methods

### LNPs Reagents

1,2-distearoyl-sn-glycero-3-phosphocholine (DSPC) and 1,2-dimyristoyl-rac-glycero-3-methoxypolyethylene glycol-2000 (DMG-PEG) were purchased from Avanti Polar Lipids (Alabaster, Albany). 33-Hydroxy-5-cholestene, 5-Cholesten-3β-ol (Cholesterol) and Acrodisc Syringe Filters with Supor Membrane, Sterile-0.2 μm, 13 mm, were purchased Sigma Aldrich (St. Louis, Missouri). COATSOME® SS-OP (SS-OP) was purchased from NOF America Corporation (White Plains, NY headquartered in Japan). Nuclease-free water, 10× PBS Buffer pH 7.4, Sodium Citrate, Dihydrate, Citric Acid, Sodium Chloride, Sucrose, Ethyl Alcohol, BD Vacutainer General Use Syringe Needles (BD Blunt Fill Needle 18G), BD Slip Tip Sterile Syringes (3 ml & 1 mL), Amicon Ultra Centifugal Filter Units, DNA-free microcentrifuge tubes (1.5 mL), Invitrogen RNase-free Microfuge Tubes (0.5 mL), Invitrogen Conical Tubes (15 mL) (DNase-RNase-free), Fisher Brand Semi-Micro Cuvette, Quant-iT™ RiboGreen® RNA Assay Kit and RnaseZap™ were purchased from Thermo Fisher Scientific (Waltham, Massachusetts). mRNA encoding luciferase was purchased from TriLink™ Biotechnologies (mRNA-Luc-Ref.: L-7602).

### LNPs Manufacturing

LNP SS-OP and LNP Lip. (VII) were manufactured using the NanoAssemblr Benchtop from Precision Nanosystems (British Columbia, Canada) according to Examples 42 and 43.

mRNA Firefly-luciferase (Trilink®) was used as nucleic acid.

The LNPs formulations was: ionizable lipid/Cholesterol/DSPC/DMG-PEG at 50/38.5/10/1.5 molar ratios.

### Animals Study

Ten weeks-old female BALB/c ByJ mice were obtained from Charles River lab (Les Oncins, Saint-Germain-Nuelles, 69210, France). At TO, animals were injected by intravenous (IV) route with 100 μl of LNPs SS-OP (group I, n=3), LNP Lip. (VII) (Group II, n=3) containing 10 μg of mRNA encoding luciferase (mRNA-Luc-Ref.: L-7602 TriLink™ Biotechnologies), or with PBS (Group III, negative control, n=3). Luciferin potassium salt

(D-luciferin, K+ salt Fluoprobes, Interchim) diluted in PBS was injected for each luminescence acquisition measure through intraperitoneal (i.p) route at 3 mg per mouse which is in large excess relative to the luciferase amount.

Optical imaging was performed using the IVIS Spectrum CT device (PerkinElmer Inc., Paris, France). Expression of the Firefly-luciferase was measured at 6 h, 24 h and 48 h post-injection of the substrate. Finally, tissues (spleen, liver, muscle, long bone, kidney, lung, heart) were sampled for ex vivo Bioluminescence Imagin (BLI). Bioluminescence quantification was performed on whole body, on anatomical segmented regions and on ex-vivo tissues using Living-Image@ software (Living Image software, PerkinElmer Inc., Paris, France). Results are expressed as total flux (ph/s) in function of time (hours) post the injection of LNPs/mRNA-Luc.

### Results

The LNPs had the following characteristics:

TABLE 5
LNPs features

| | | | | | mRNA final |
|---|---|---|---|---|---|
| | Ionizable | Diameter | Polydispersity | Encapsulation | concentration |
| LNP | lipid | (nm) | Index (PDI) | rate (%) | (μg/mL) |
| LNP-SSOP | SS-OP | 121 | 0.147 | 95 | 100 |
| LNP Lip. | compound | 108 | 0.086 | 99 | 100 |
| (VII) | of formula | | | | |
| | (VII) | | | | |

The diameter size, the polydispersity index, and the encapsulation rate were determined as indicated in Example 43.

The LNPs obtained with a lipidic compound of the invention had reduced diameter and improved homogeneous distribution of size.

It is known that particulates of large size may induce adverse reactions at the time of the injection, such as pain. The reduced and homogeneous size of the LNPs of the invention allow reducing risk of adverse effects.

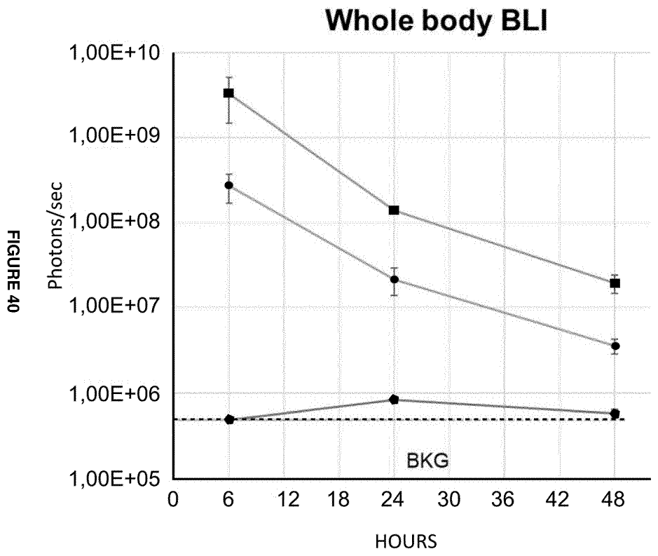

As shown on FIG. 40, the highest whole body bioluminescent signal is observed at 6 h post injection for all LNPs. The whole-body bioluminescent signal obtained with the LNPs SS-OP and the LNPs Lip. (VII) were quite comparable.

LNPs exhibited different biodistributions: a high level of liver expression for LNP SSOP and a more diffused signal for LNP Lip. (VII) at 6 h post injection were observed.

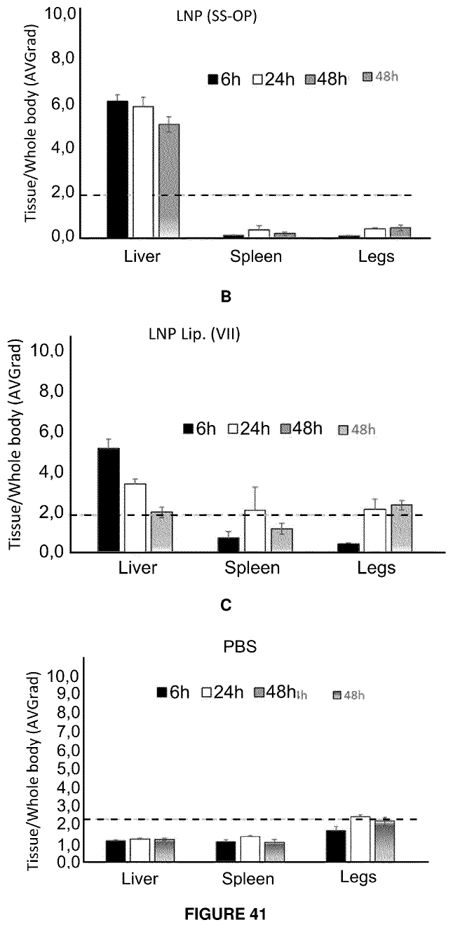

As shown on FIG. 41A (LNPs SS-OP), FIG. 41B (LNPS Lip. (VII)), and FIG. 41C (PBS), LNP lip. (VII) exhibits a high level of expression within liver as early as 6h post-injection.

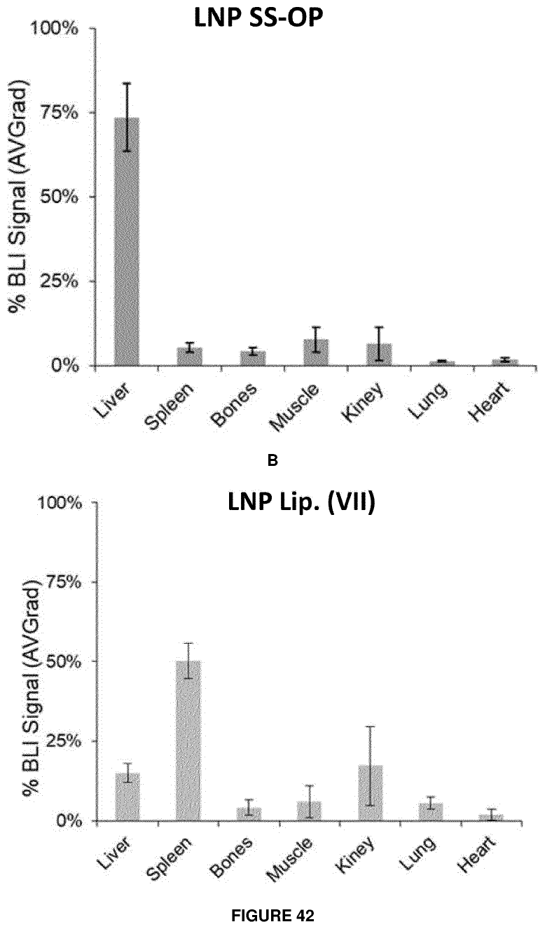

As shown on FIG. 42A (LNPs SS-OP) and FIG. 42B (LNPS Lip. (VII)), the difference in LNPs structures and related size were associated with differences in spontaneous tissues distribution, which could influence further specific targeting efficacy:
LNP SS-OP were mainly distributed in liver with high transcription efficiency, and
LNP Lip. (VII) showed the broader distribution (liver, spleen, myeloid tissues in long bones) with intermediate transcription efficiency

### Example 45: Formulation of hEPO mRNA in LNPs comprising lipidic compounds of the disclosure

### Material & Methods

### Reagents and LNPs Preparation

CleanCap® EPO mRNA (5moU), a non-replicative, highly purified, mRNA encoding the human erythropoietin was obtained from TriLink Biotechnologies, San Diego, CA (catalogue number L7209; hEPO mRNA). This mRNA is capped using CleanCap, TriLink's proprietary co-transcriptional capping method, which results in the naturally occurring Cap 1 structure with high capping efficiency. It is polyadenylated, modified with 5-methoxyuridine and optimized for mammalian systems. It mimics a fully processed mature mRNA.

LNPs comprising ionizable lipid of formula (VI), (VII), (VIII), (XI), or (XII), DSPC, Cholesterol (Chol), and DMG-PEG2000, and at a molar ratio of 50:10:38.5:1.5, and hEPO mRNA at a N/P charge ratio=6, were prepared using the NanoassembIR as indicated in Example 42. The obtained LNPs are referred to as LNPs Lip. (VI), LNPs Lip. (VII), LNPs Lip. (VIII), LNPs Lip. (XI), and LNPs Lip. (XII).

An Aqueous/Ethanolic phase volume ratio of 3/1 and at total flow rate of 4 mL/min was used. LNPs were prepared at a concentration of 60 μg of hEPO mRNA/mL in PBS 1X.

Before administration, LNPs were resuspended in a PBS buffer (pH 7.4) in a volume adjusted for attaining 1 μg of mRNA per dose.

### Animals

Female Balb/c ByJ mice (7 weeks of age at receipt) were purchased from Charles River Laboratories (Saint-Germain-Nuelles, France) and housed for one-week acclimation before starting the study. Mice were identified individually by fur coloration. Experiments were approved by Sanofi Pasteur's animal ethics committee and followed European guidelines for standards of animal care.

### Study Schedule

Four 8-week-old mice per group were injected on DO via intramuscular route in the quadriceps with 1 μg-dose of hEPO mRNA formulated in LNPs Lip. (VI), (VII), (VIII), (XI), or (XII) under a final volume of 50 μL. As negative control, 4 mice received the same volume of PBS (for accelerated stability study and lipid screening) or Citrate Buffer (lipid screening).

Blood samples were collected 6 hours post-injection to measure the expression of hEPO in serum using a specific ELISA assay.

### Blood Samples

Blood samples were collected 6 hours post-injection by carotid section under deep anesthesia with Imalgène/Rompun (1.6 mg of Ketamine/0.32 mg of Xylazine) in serum-separation tubes (BD Vacutainer #BD367957). Sera were aliquoted and stored at −20° C. until hEPO determination.

hEPO Determination in Mouse Serum

hEPO expression in mouse sera was assessed using human Erythropoietin Quantikine IVD ELISA kit (R&D Systems #DEP00). The ELISA was performed following supplier's instructions. Briefly, sera were added in pre-coated plates and incubated for one hour at room temperature under orbital shaking. After sera removal, Erythropoietin conjugate was added for one hour at room temperature under orbital shaking. Plates were washed and Substrate solution was added for 20-25 minutes at room temperature before stopping the reaction with Stop solution. Absorbance at 450 nm with 650 nm signal subtraction was determined in a microplate reader. Data were analyzed using SoftmaxPro software and expressed in log 10 of the concentration of hEPO measured in mouse sera in pg/mL.

### Results

The amount of plasma EPO according to the LNPs Lip. were as follows:

TABLE 6
hEPO expression in mice having received intramuscular injection
of LNPs Lip/DSPC, or a given analogue, containing hEPO mRNA

| | EPO secretion IM |
|---|---|
| LNPs lip. | (Log10 pg/ml) |
| LNPs lip. (VI) | 1.5 |
| LNPs lip. (VII) | 3.4 |
| LNPs lip. (VIII) | 1.8 |
| LNPs lip. (XI) | 1.3 |
| LNPs lip. (XII) | 1.9 |

### Example 46: Formulation of hEPO mRNA in LNPs comprising lipidic compounds of the disclosure

### Material & Methods

### LNPs Reagents

LNPs reagents as described in Example 42 were used in the present example, except that a hEPO mRNA were used instead.

A non-replicative mRNA encoding hEPO UNR was used. The hEPO mRNA was produced by in vitro transcription (IVT) as an unmodified mRNA transcript from a linear DNA template generated by PCR, using wild type bases and T7 RNA polymerase (Avci-Adali et al (J. Vis. Exp. (93), e51943, doi: 10.3791/51943 (2014) and Kwon et al., Biomaterials 156 (2018) 172e193). The mRNA was 3′ polyadenylated (A120) and 5′ capped (Cap 1).

After Dnase and phosphatase treatment, the mRNA was purified to high degree of purity by silica membrane filtration followed by HPLC. mRNA was packaged as 1 mL aliquots of 1 mg/mL solution in 1 mM Sodium Citrate, pH 6.4.

### LNPs Manufacturing

LNPs comprising ˜30-40 μg/mL of mRNA encoding a hEPO (internal IVT mRNA) were prepared with lipidic compound of formula (XIV), (XVI), (XVIII), (XIX), (XXX), (XXXI), (XXXII), (XXXIII), (XXXIV), (XXXV), (XXXVI), (XXXVII), (XXXVIII), and (DLIN-MC3-DMA CAS NO.: 1224606) (“MC3” used as comparative), DSPC, Cholesterol (Chol), and DSPC-PEG2000, and at a molar ratio of 50:10:38.5:1.5, and hEPO mRNA at a N/P charge ratio=6 and were prepared using the Nanoassembler®.

The obtained LNPs are referred to as LNPs Lip. (XIV), LNPs Lip. (XVI), LNPs Lip. (XVIII), LNPs Lip. (XIX), LNPs Lip. (XXX), LNPs Lip. (XXXI), LNPs Lip. (XXXII), LNPs Lip. (XXXIII), LNPs Lip. (XXXIV), LNPs Lip. (XXXV), LNPs Lip. (XXXVI), LNPs Lip. (XXXVII), LNPs Lip. (XXXVIII), and LNPs Lip. MC3.

An Aqueous/Ethanolic phase volume ratio of 3/1 and at total flow rate of 4 mL/min was used. LNPs were prepared at a concentration of 60 μg of hEPO mRNA/mL in PBS 1X.

Before administration, LNPs were resuspended in a PBS buffer (pH 7.4) in a volume adjusted for attaining 0.1 μg of mRNA per dose (50 μl).

### Animals

As in Example 45.

### Study Schedule

As in Example 45, except that LNPs Lip. (XIV), LNPs Lip. (XVII), LNPs Lip. (XVIII), LNPs Lip. (XIX), LNPs Lip. (XXX), LNPs Lip. (XXXI), LNPs Lip. (XXXII), LNPs Lip. (XXXIII), LNPs Lip. (XXXIV), LNPs Lip. (XXXV), LNPs Lip. (XXXVI), LNPs Lip. (XXXVII), LNPs Lip. (XXXVIII), and LNPs Lip. (MC3) were used.

### Blood Samples

Blood samples were collected 6 hours post-injection by carotid section under deep anesthesia with Imalgène/Rompun (1.6 mg of Ketamine/0.32 mg of Xylazine) in serum-separation tubes (BD Vacutainer #BD367957). Sera were aliquoted and stored at −20° C. until hEPO determination.

hEPO Determination in Mouse Serum

As in Example 45.

The size, encapsulation rate and polydispersity index (PDI) were measured as follows.

### RNA Titration/Encapsulation Rate

The percentage of encapsulated mRNA and concentration of mRNA in LNPs were measured using the Quant-iT Ribogreen RNA reagent kit according to manufacturer recommendations (Invitrogen Detection Technologies) and quantified with a fluorescent microplate reader or a standard spectrophotometer using fluorescein excitation and emission wavelength.

For quantification of non-encapsulated RNA, LNPs were diluted in Tris/EDTA assay buffer (10 mM Tris-HCl, 1 mM EDTA, pH 7.5). For quantification of total amount of RNA, LNPs were diluted in Tris/EDTA assay buffer (10 mM Tris-HCl, 1 mM EDTA, pH 7.5) containing 0.5% (v/v) Triton X100. Ribogreen dye (200X diluted) was added to the samples (50/50 mix; sample/Ribogreen reagent), mixed thoroughly and incubated 5 min at room temperature in the dark.

Fluorescence was measured on the plate reader.

### Lipid Quantification

It was assumed that there was no loss of lipids during the formulation process. The total lipid concentration before dialysis was then assumed to be 5 mg/mL. The final lipid concentration was defined by taking into account the dilution factor occurring during the dialysis step.

### Particle Size Distribution, Polydispersity Index Zeta Potential, and Osmolarity

Zeta potential and particle size distribution of LNPs were measured by using a zeta sizer Nano ZS light scattering instrument (Malvern Instruments) according to manufacturer recommendations.

Particle sizes were reported as the Z-average size (harmonic intensity averaged particle diameter) along with the Polydispersity Index (PDI), an indicator of the “broadness” of the particle size distribution. Samples were diluted to 1/100 in phosphate buffered saline (PBS) before measurement. For accurate particle sizing with the Nano ZS, the viscosity of the buffer and the refractive index of the material had to be provided to the equipment software (PBS: v=1.02 cP, RI=1.45).

For zeta potential measurements, samples were diluted in water (v=0.8872 cP). Data were analyzed using the Zetasizer Software V 7.11 from Malvern

The osmolarity and the pH of formulations were measured by using respectively a micro-sample Osmometer (Fiske Associates model) and a pH meter (Mettler Toledo) according to the equipment manufacturer instructions.

### Results

The amount of plasma EPO according to the LNPs Lip. were as follows:

TABLE 7
hEPO expression in mice having received intramuscular injection
of LNPs Lip/DSPC, or a given analogue, containing hEPO mRNA

| | | EPO plasma |
|---|---|---|
| LNPs lip. (No.) | Example | secretion (ng/ml) |
| LNPs lip. (XIV) | Example 9 | 2.5 |
| LNPs lip. (XVI) | Example 11 | 3.4 |
| LNPs lip. (XVIII) | Example 13 | 7.1 |
| LNPs lip. (XIX) | Example 14 | 1.7 |
| LNPs lip. (XXX) | Example 22 | 2.5 |
| LNPs lip. (XXXI) | Example 23 | 2.0 |
| LNPs lip. (XXXII) | Example 24 | 2.4 |
| LNPs lip. (XXXIII) | Example 25 | 5.1 |
| LNPs lip. (XXXIV) | Example 26 | 4.0 |
| LNPs lip. (XXXV) | Example 27 | 2.7 |
| LNPs lip. (XXXVI) | Example 28 | 2.8 |
| LNPs lip. (XXXVII) | Example 29 | 1.8 |
| LNPs lip. (XXXVIII) | Example 30 | 7.0 |
| LNPs Lip. (MC3) | | 6.1 |

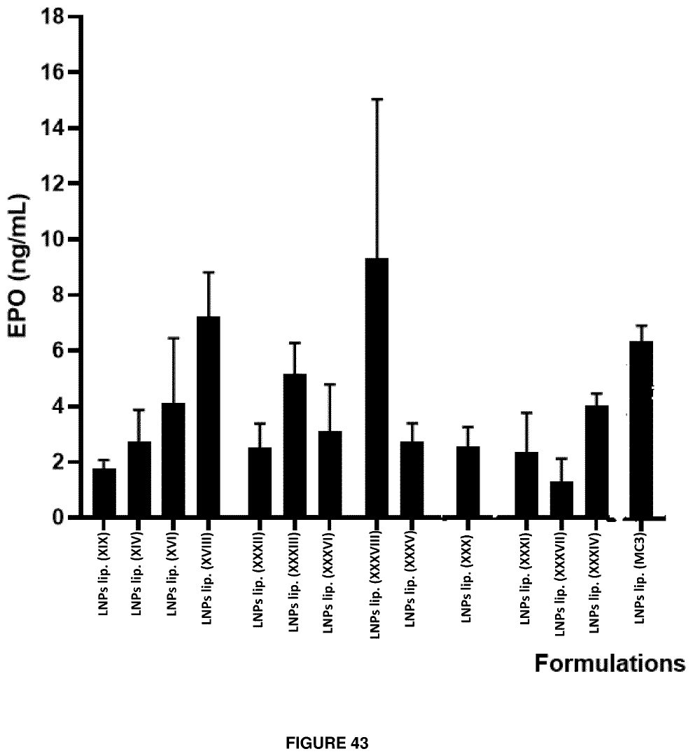

Data are represented on FIG. 43.

The prepared LNPs had the following formulations and characteristics:

TABLE 8
LNPs formulations & characteristic

| LNPs lip. (No.) | Lipids components | Lipids ratio | N/P |
|---|---|---|---|
| LNPs lip. (XIV) | Lipid/Chol/DSPC/ | 50/38.5/10/1.5 | 6 |
| LNPs lip. (XVI) | DSPC-PEG | | |
| LNPs lip. (XVIII) | | | |
| LNPs lip. (XIX) | | | |
| LNPs lip. (XXX) | | | |
| LNPs lip. (XXXI) | | | |
| LNPs lip. (XXXII) | | | |
| LNPs lip. (XXXIII) | | | |
| LNPs lip. (XXXIV) | | | |
| LNPs lip. (XXXV) | | | |
| LNPs lip. (XXXVI) | | | |
| LNPs lip. (XXXVII) | | | |
| LNPs lip. (XXXVIII) | | | |
| LNPs Lip. (MC3) | | | |

TABLE 9
LNPs formulations & characteristic

| | Size LNP | Polydispersity | % |
|---|---|---|---|
| LNPs | (nm) | Index (PDI) | encapsulation |
| LNPs lip. (XIX) | 104 | 0.120 | >98% |
| LNPs lip. (XIV) | 65 | 0.120 | >98% |
| LNPs lip. (XVII) | 73 | 0.010 | >98% |
| LNPs lip. (XVIII) | 119 | 0.051 | >98% |
| LNPs lip. (XXXII) | 61 | 0.110 | >98% |
| LNPs lip. (XXXIII) | 65 | 0.100 | >98% |
| LNPs lip. (XXXVI) | 78 | 0.150 | >98% |
| LNPs lip. (XXXVIII) | 99 | 0.100 | >98% |
| LNPs lip. (XXXV) | 82 | 0.170 | >98% |
| LNPs lip. (XXX) | 100 | 0.080 | >98% |
| LNPs lip. (XXXVII) | 105 | 0.180 | >98% |
| LNPs lip. (XXXIV) | 82 | 0.170 | >98% |
| LNPs lip. (XXX) | 120 | 0.180 | >98% |
| LNPs Lip. (MC3) | 106 | 0.150 | >98% |

### Example 47: Immunogenicity of LNPs comprising influenza HA mRNA in mice

The aim of the study was to evaluate the immunogenicity induced with different LNPs made with different lipidic compounds as disclosed herein and containing non-replicative mRNA encoding full-length hemagglutinin (HA) of influenza virus.

### Material & Methods

### Reagents and LNPs Preparation

LNPs were prepared as described in Example 42 and were always composed of ionizable lipid/DSPC/Chol/DMG-PEG2000 at a 50:10:38.5:1.5 molar ratio. Ratio N/P was always equal to 6.

Lipid compounds of formulation (VII), (VIII) and (XII) were used. LNPs are referred to as LNP Lip. (VII), LNP Lip. (VIII) and LNP Lip. (XII).

A natural, non-replicative mRNA encoding full-length hemagglutinin (HA) of influenza virus strain A/California/07/09 (H₁N1) was used.

The amounts of mRNA and cationic lipid were adjusted to reach the indicated cationic (N)/anionic (P) charges ratio.

### Animals and Schedule of Administration

BALBc/ByJ mice (8 weeks old at DO; 8 per group) were immunized with 5 μg of natural, non-replicative mRNA encoding full-length hemagglutinin (HA) of influenza virus strain A/California/07/09 (H₁N1) formulated in the 3 different lipid nanoparticles: LNP Lip. (VII), LNP Lip. (VIII) and LNP Lip. (XII).

Six groups of 8 BALBc/ByJ mice (8 weeks old at DO) received two intramuscular (IM) injections, given three weeks apart, of either 1 or 5 μg of mRNA with each the 4 LNPs formulations.

A negative control group of mice was immunized with a citrate buffer.

HI titers were measured 3 weeks following the second immunization.

### Determination of Hemagglutination Inhibiting Antibody Titers (HI Titers)

This technique is used to titrate the functional anti-HA antibodies present in the sera of influenza immunized animals, on the basis of the ability of a serum containing specific functional antibodies directed against HA to inhibit the influenza virus hemagglutination activity.

Serial dilutions of virus (clarified allantoic fluid) A/H₁N1/California/7/2009 strain were performed in PBS in order to calibrate the viral suspension and to obtain 4 HAU (Hemagglutination Unit) in presence of cRBCs (0.5% in PBS). Calibrated virus (50 μL) was then added to the V shaped well of a 96 well plate on 50 μL of serum serial dilutions (2-fold) in PBS starting from 1:10 and incubated one hour at room temperature.

In order to eliminate serum non-specific inhibitors directed against the HA, each serum was treated with a receptor-destroying enzyme (RDE) (neuraminidase from Vibrio cholerae-Type III-Sigma Aldrich N7885) and with chicken red blood cells (cRBCs). Briefly, 10 mU/mL of RDE was added to each serum. The mix was then incubated 18 h at 37° C., followed by 1 h inactivation at 56° C. To cool, the mixture “serum-RDE” was placed for a time ranging from 30 min to 4 hours at 4° C. The “serum-RDE” mixture was then absorbed on 10% cRBCs in PBS for 30 min, at room temperature, and then centrifuged at 5° C., 10 min at 700 g. The supernatant corresponding to 10-fold diluted serum was collected to perform the HI assay.

Chicken red blood cells (0.5% in PBS) (50 μL) were then added to each well and inhibition of hemagglutination or hemagglutination was visually read after one hour at room temperature.

The titer in HI antibody is the reciprocal of the last dilution giving no hemagglutination. A value of 5 corresponding to half of the initial dilution (1:10) was arbitrary given to all sera determined negative in order to perform statistical analysis.

### Results

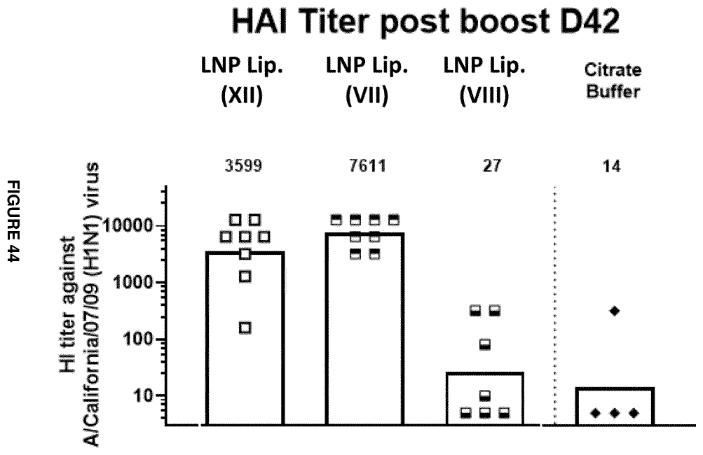

As shown on FIG. 44, mice injected with LNP Lip. (VII) and LNP Lip. (XII) containing HA mRNA were able to induce a strong antibody response. The immune response obtained with LNP Lip. (VIII) was weaker, but still above control.

1. A lipidic compound of formula (I):
A—(CH₂)ₙ—CX—B—Z—R1 (I)
wherein:
R1 represents a C₁₀ to C₅₇ lipophilic or hydrophobic tail-group, wherein R1 is an optionally substituted, branched or unbranched linear, saturated or unsaturated, C₁₀ to C₅₅ hydrocarbon radical, and which hydrocarbon skeleton that is optionally interrupted by one or several atoms of oxygen or nitrogen and/or one or several moiety —(C═O)—, —O—(C═O)— or —(C═O)—O— and which one nitrogen atom, if present in the skeleton, can be linked, directly or not, to said Z radical;
Z is a spacer arm having from 2 to 24, for instance from 2 to 18, for example from 4 to 12 carbon atoms in an or unbranched linear saturated or unsaturated hydrocarbon chain, said chain that is interrupted by one or several atoms of oxygen and/or moieties selected among —S—S—; —(O═C)—; —(C═O)—O—; —O—(O═C)—; —S—; —NH—, —NH—(O═C)—; —(O═C)—NH— and —NH—(C═O)—O— and for instance by —(C═O)—O—; —O—(O═C)— and —NH—(C═O)—O— and optionally having an oxygen atom or a moiety selected among —NH—(O═C)—*—O—(O═C)—*; —(C═O)—O—*; and —(O═C)— to its end linked to the hydrophobic tail-group, with * indicating the single bond linking said moiety to the hydrophobic tail-group;
B represents an oxygen atom or a —NH— group;
X is an oxygen atom or a sulfur atom;
n is 0, 1, 2, 3, 4, 5 or 6; and
A represents a group selected in the group consisting of:
a R2R3N-group in which R2 and R3 represent independently of each other a linear or branched (C₁-C₆) alkyl group,
a NR2R3-Alk-Y-group in which Y is an oxygen or a nitrogen atom, Alk is an alkylene moiety in C₂ to C₆ and R2 and R3 represent independently of each other a linear or branched (C₁-C₆) alkyl group,
a 4- to 8-membered saturated heterocyclic radical comprising 3 to 7 carbon atoms and 1 or 2 nitrogen atoms, said 4- to 8-membered saturated heterocyclic radical being linked to the rest of the molecule by a carbon atom or a nitrogen atom and being optionally substituted by 1 to 4 substituents, independently of each other, selected from a linear or branched (C₁-C₆) alkyl group;
or one of its pharmaceutically acceptable salts thereof; and with said compound that is in all the possible racemic, enantiomeric and diastereoisomeric isomer forms.

2. The compound according to claim 1 that is under a cationic form.

3. The compound of anyone of claim 1 or 2, wherein R1 represents a group selected in the group consisting of:

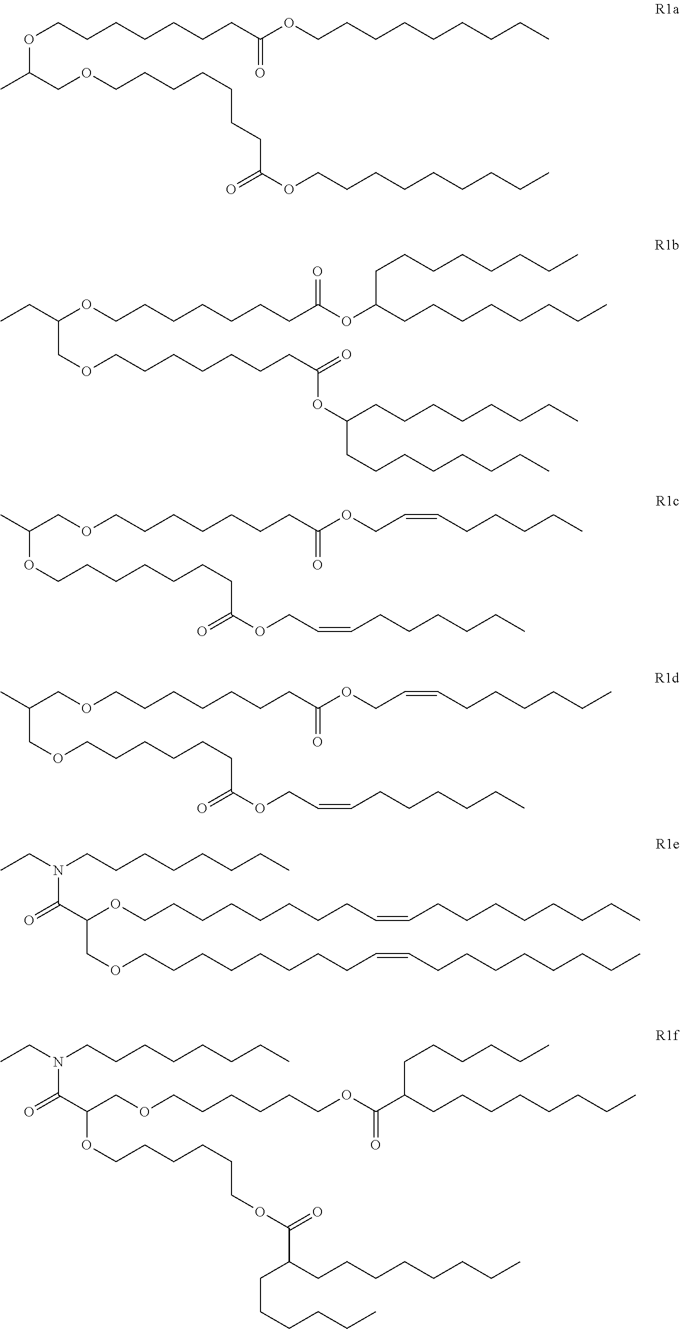

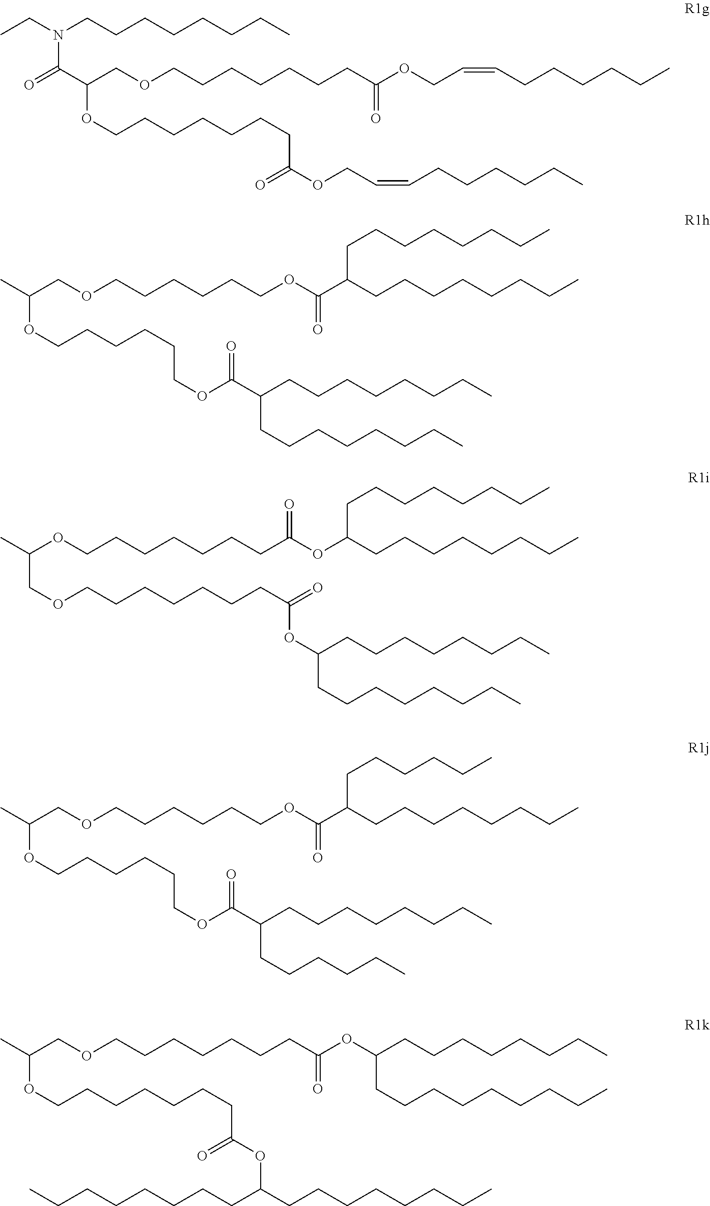

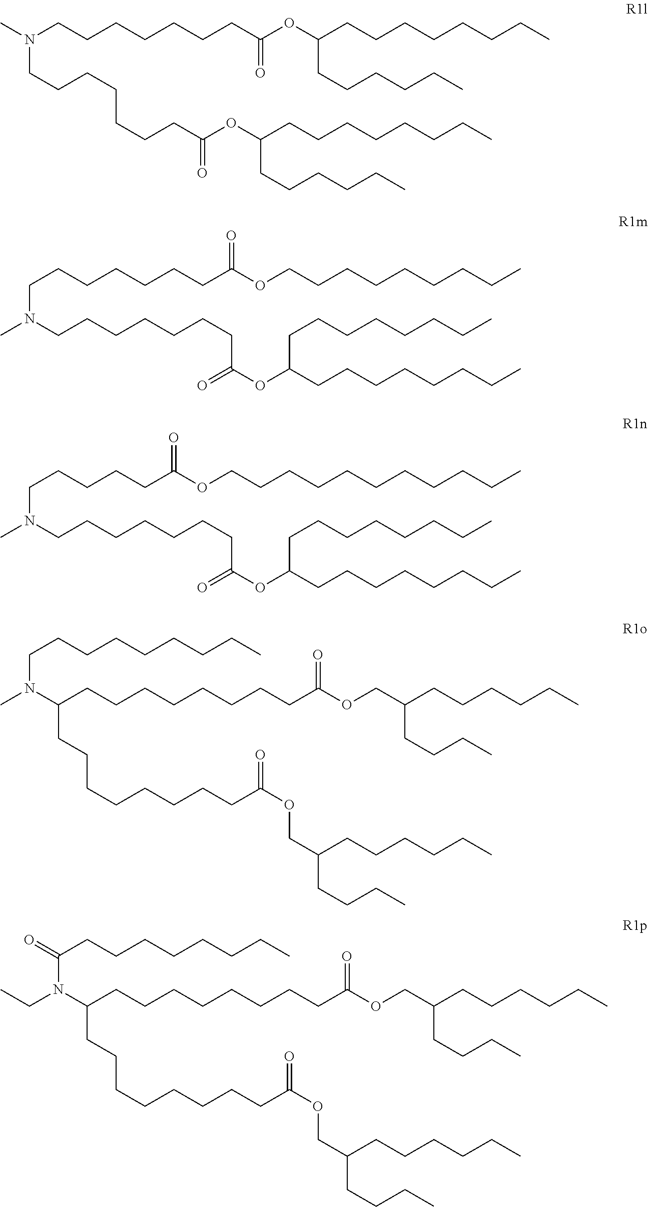

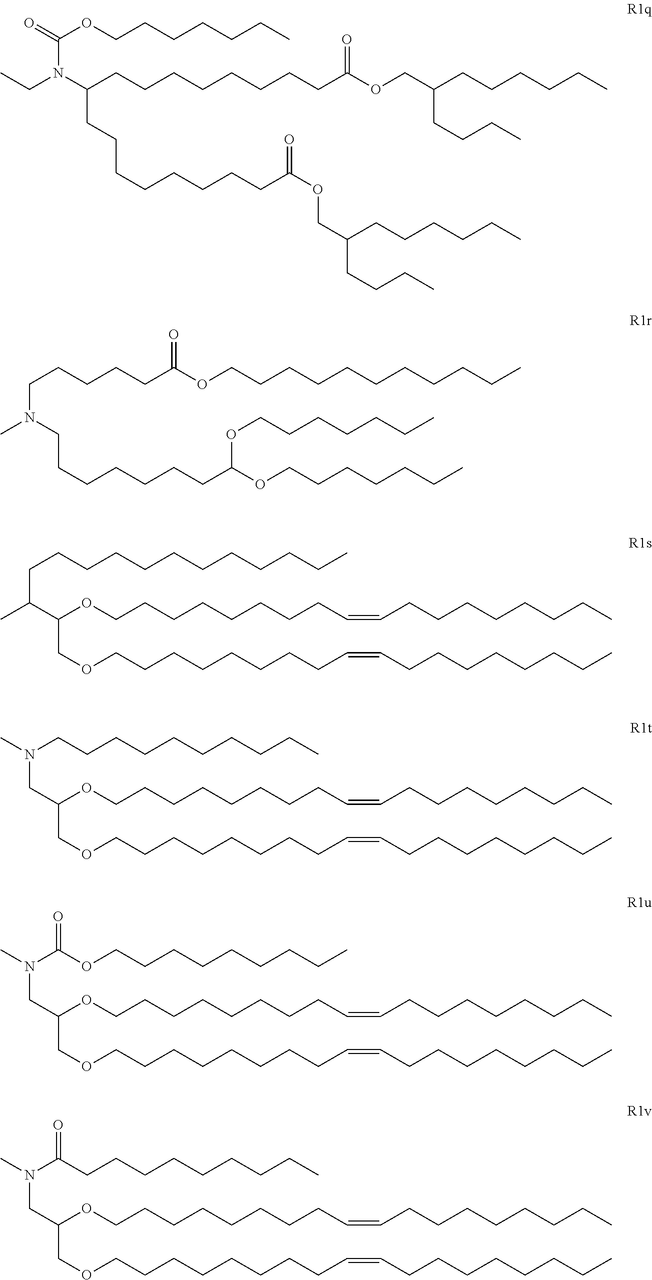

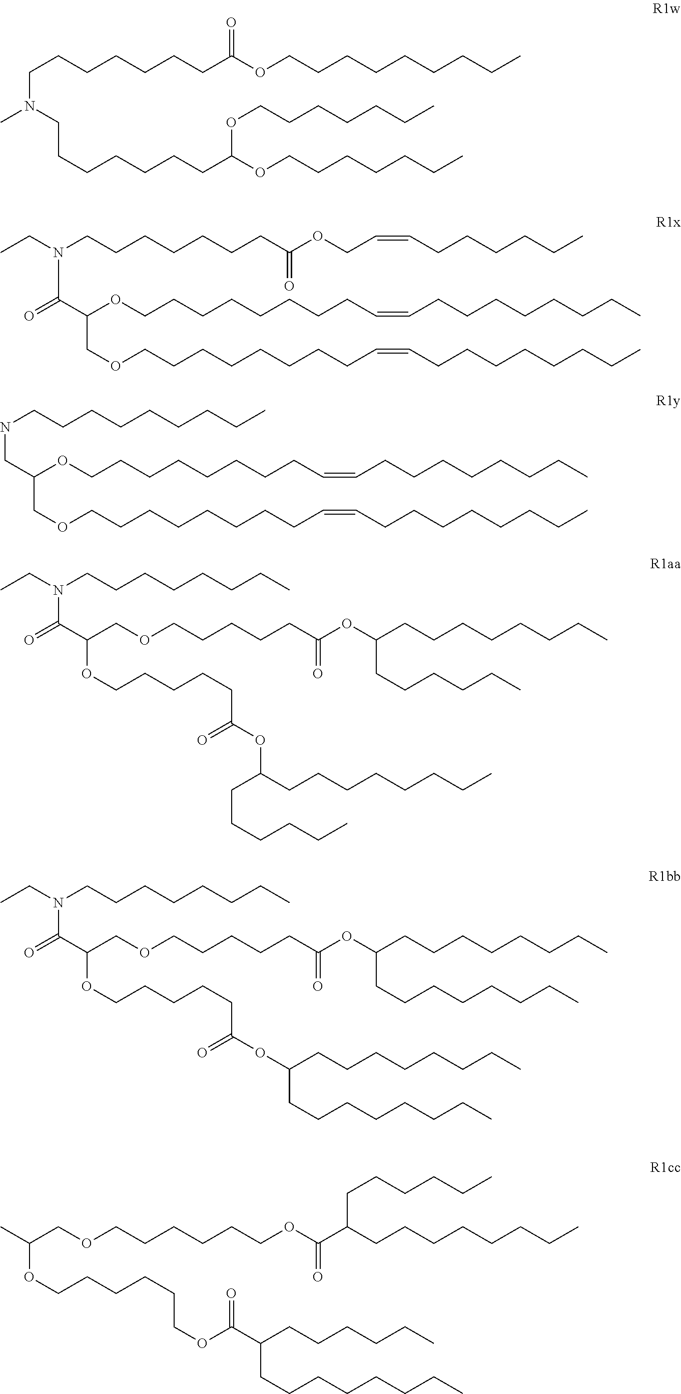

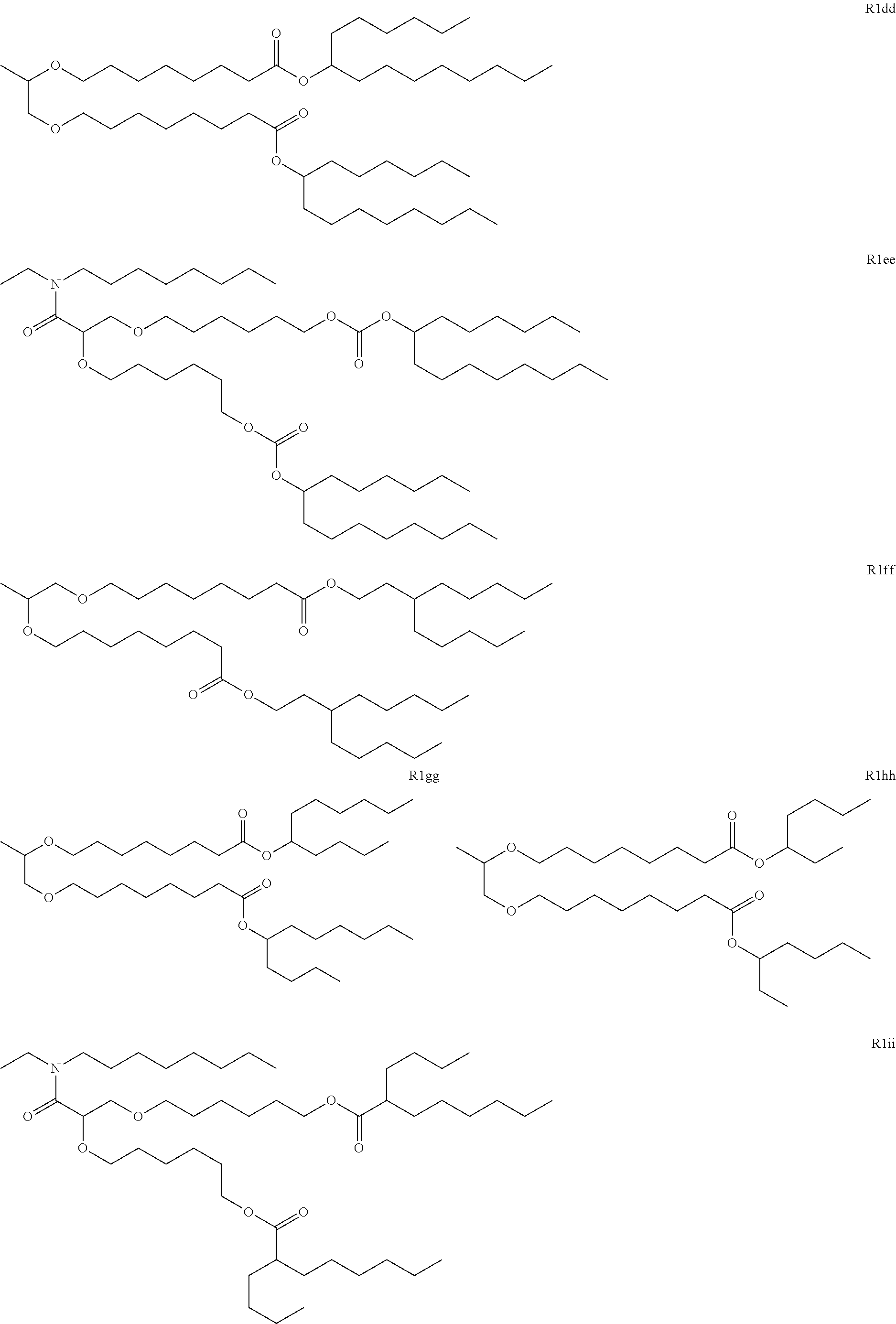

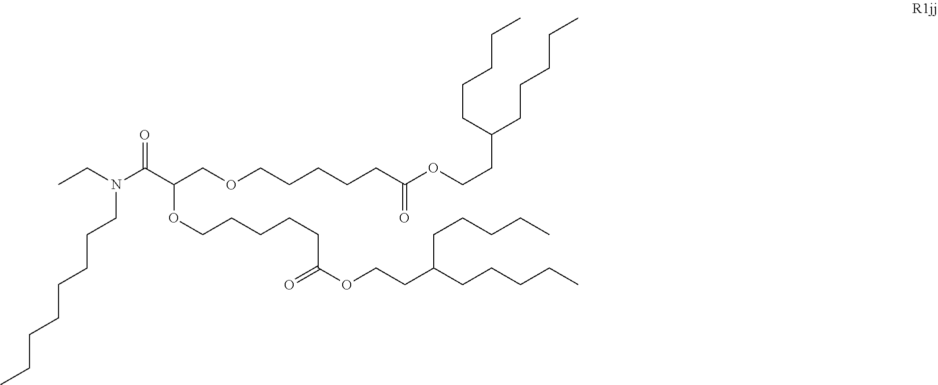 [SMILES: CCCCCCCCN(CC)C(=O)C(COCCCCCC(=O)OCCC(CCCCC)CCCCC)OCCCCCC(=O)OCCC(CCCCC)CCCCC]

4. The compound according to anyone of claims 1 to 3, wherein Z represents a radical of formula
—((CH₂)₂—O)ₘ—(CH₂)ᵣ(T)_{q}—*(Z)
with
* indicating the single bond linking said radical to the hydrophobic tail-group,
m being an integer from 1 to 12, for instance from 2 to 4, for example 4,
r being zero or an integer from 1 to 4,
q being zero or 1 and
T being selected in the group consisting of —(O═C)—; —(C═O)—O—**; —O—(O═C)—**; and —NH—(C═O)—O—** with ** indicating the single bond linking said group to the hydrophobic tail-group.

5. The compound according to anyone of claims 1 to 4, wherein B is an oxygen atom.

6. The compound according to anyone of claims 1 to 4, wherein B is a —NH— group.

7. The compound according to anyone of claims 1 to 6, wherein X is an oxygen atom.

8. The compound according to anyone of claims 1 to 7, wherein n is 0, 1, 2, 3 or

4.

9. The compound according to anyone of claims 1 to 9, wherein A is selected in the group consisting of —N(CH₃)₂, —N(CH₂—CH₂—CH₃)₂, O—(CH₂)₂ N (CH₃)₂, N—(CH₂)₂ N(CH₃)₂ and NR2R3-Alk-Y-group in which Y is an oxygen or a nitrogen atom, Alk is an alkylene moiety in C₂ to C₆ and R2 and R3 represent independently of each other a linear or branched (C₁-C₆) alkyl group,

10. The compound according to anyone of claims 1 to 8, wherein A represents a 4- to 8-membered saturated heterocyclic radical comprising 3 to 7 carbon atoms and 1 or 2 nitrogen atoms, said 4- to 8-membered saturated heterocyclic radical being linked to the rest of the molecule by a carbon atom or a nitrogen atom and being optionally substituted by 1 to 4 substituents, independently of each other, selected from a linear or branched (C₁-C₆) alkyl group.

11. The compound according to anyone of claims 1 to 10 having an apparent pKa lower than 7, or ranging from 4.5 to 7.

12. The compound according to anyone of claims 1 to 8 and 10 wherein said compound is of formula (II) 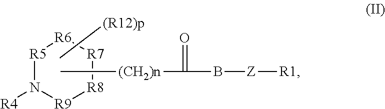
wherein
Z, n and R1 are as defined in anyone of claims 1 to 4 and 8,
R4 is a (C₁-C₅) alkyl group, for instance a (C₁-C₄) alkyl group, such as a methyl group;
R12 is a (C₁-C₅) alkyl group, for instance a (C₁-C₄) alkyl group, such as methyl group;
p is equal to zero or 1, and for instance p is equal to zero;
R5, R6, R7, R8 and R9 are independently one of each other a moiety selected among —CH2—; —CHR12— and —NH—, and the one of R5, R6, R7, R8 and R9 involved in the linkage with the rest of the molecule, being a moiety selected among —CH—; —CR12—and —N— and with the proviso that only one of R5, R6, R7, R8 and R9 is —NH— or —N—; and
B represents an oxygen atom or a —NH— group, for instance an oxygen atom, or one of its pharmaceutically acceptable salts thereof; and with said compound that is in all the possible racemic, enantiomeric and diastereoisomeric isomer forms.

13. The compound according to anyone of claims 1 to 8, 10 and 12, wherein said compound is of formula (IIa) 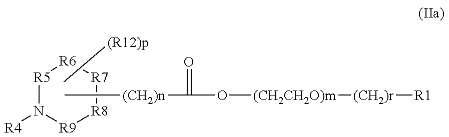
wherein
R1 is as defined in anyone of claims 1 to 3,
n is 0, 1, 2, 3, 4, 5 or 6, for instance 0 to 4, such as 0, 1 or 2;
r is 0, 1, 2, 3 or 4, for instance 0, 1 or 2;
R4 to R9, R12 and p are as defined in previous claim and for instance p is equal to zero,
m is an integer from 1 to 12, for instance from 2 to 6, for example 4; or one of its pharmaceutically acceptable salts thereof; and with said compound that is in all the possible racemic, enantiomeric and diastereoisomeric isomer forms.

14. The compound according to anyone of claims 1 to 8 and 10, wherein said compound is of formula (III) 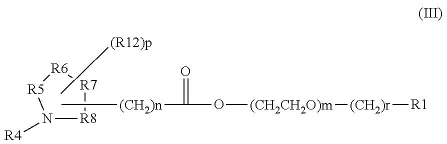
wherein
R1 is as defined in anyone of claims 1 to 3,
n is 0, 1, 2, 3, 4, 5 or 6, for instance 0 to 4, such as 0, 1 or 2.
m is an integer from 1 to 12, for instance from 2 to 6, for example 4;
r is 0, 1, 2, 3 or 4, for instance 0, 1 or 2 and;
R4 to R8, R12 and p are as defined in claim 13 and for instance p is equal to zero or one of its pharmaceutically acceptable salts thereof; and with said compound that is in all the possible racemic, enantiomeric and diastereoisomeric isomer forms.

15. The compound according to anyone of claims 1 to 9 and 11, wherein said compound is of formula (IV): 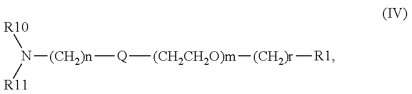
wherein
R1 is as defined in anyone of claims 1 to 3;
Q is a moiety selected in the group consisting of —(C═O)—O*; —O(C═O)O*; —N(C═O)O—* and —O(C═O)N—* with * indicating the linking to the moiety (CH₂CH₂O) m
n is 0, 1, 2, 3, 4, 5 or 6, for instance 1 to 5, for example 2, 3 or 4;
r is 0, 1, 2, 3 or 4, for instance 0, 1 or 2;
I R10 and R11 represent independently of each other a (C₁-C₅) alkyl group, for instance a (C₁-C₄) alkyl group, such as a methyl group or a propyl group; and
m is an integer from 1 to 12, for instance from 2 to 6, and for example 4;
or one of its pharmaceutically acceptable salts thereof; and with said compound that is in all the possible racemic, enantiomeric and diastereoisomeric isomer forms.

16. The compound according to anyone of claims 1 to 9,11 and 15, wherein said compound is of formula (V) 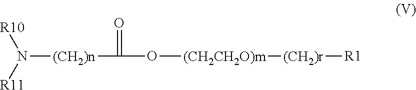
Wherein
R1, R10, R11, n, m and r areas defined in claim 15,
or one of its pharmaceutically acceptable salts thereof; and with said compound that is in all the possible racemic, enantiomeric and diastereoisomeric isomer forms.

17. The compound of anyone of claims 1 to 16, wherein said compound is selected in the group consisting of: 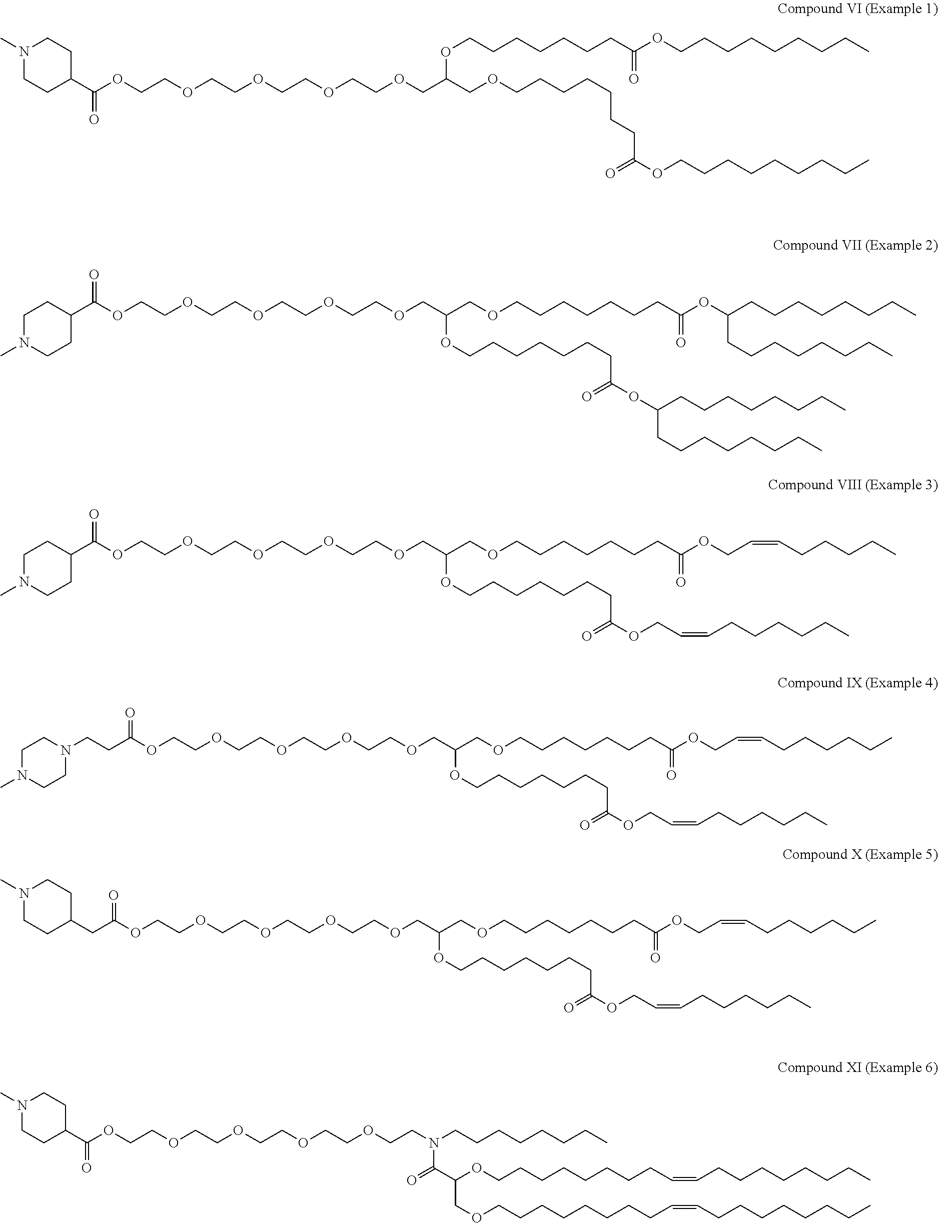 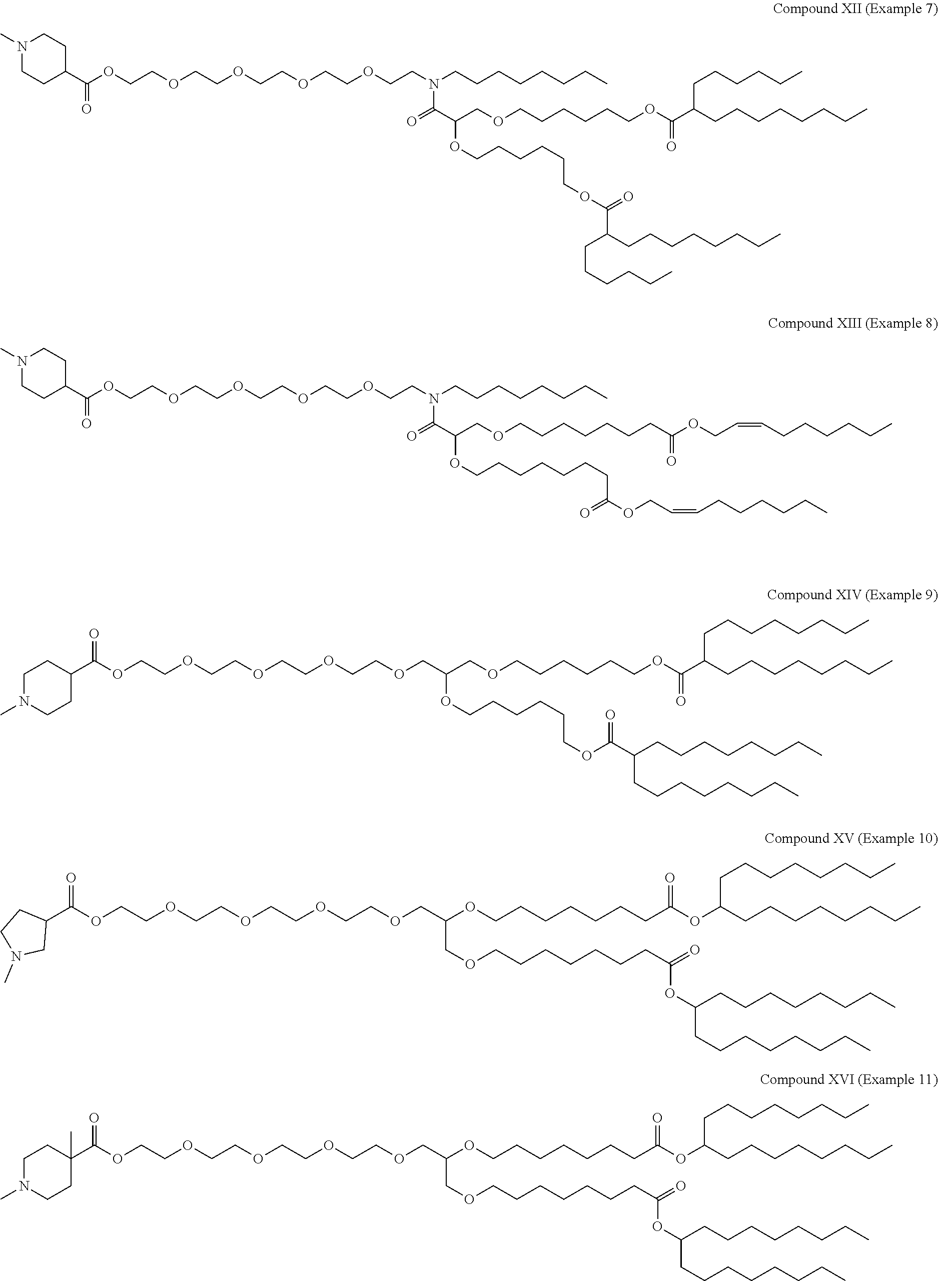 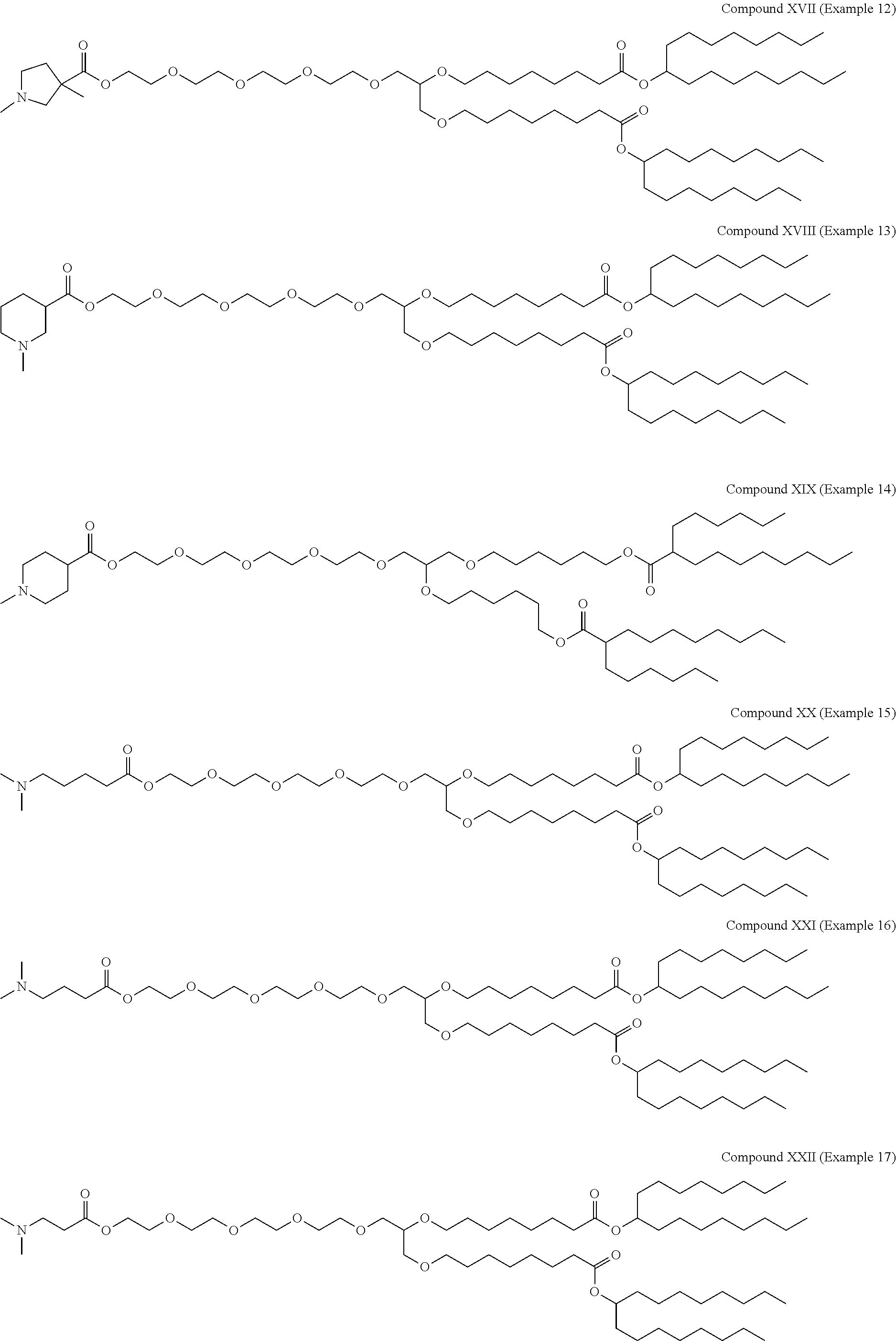 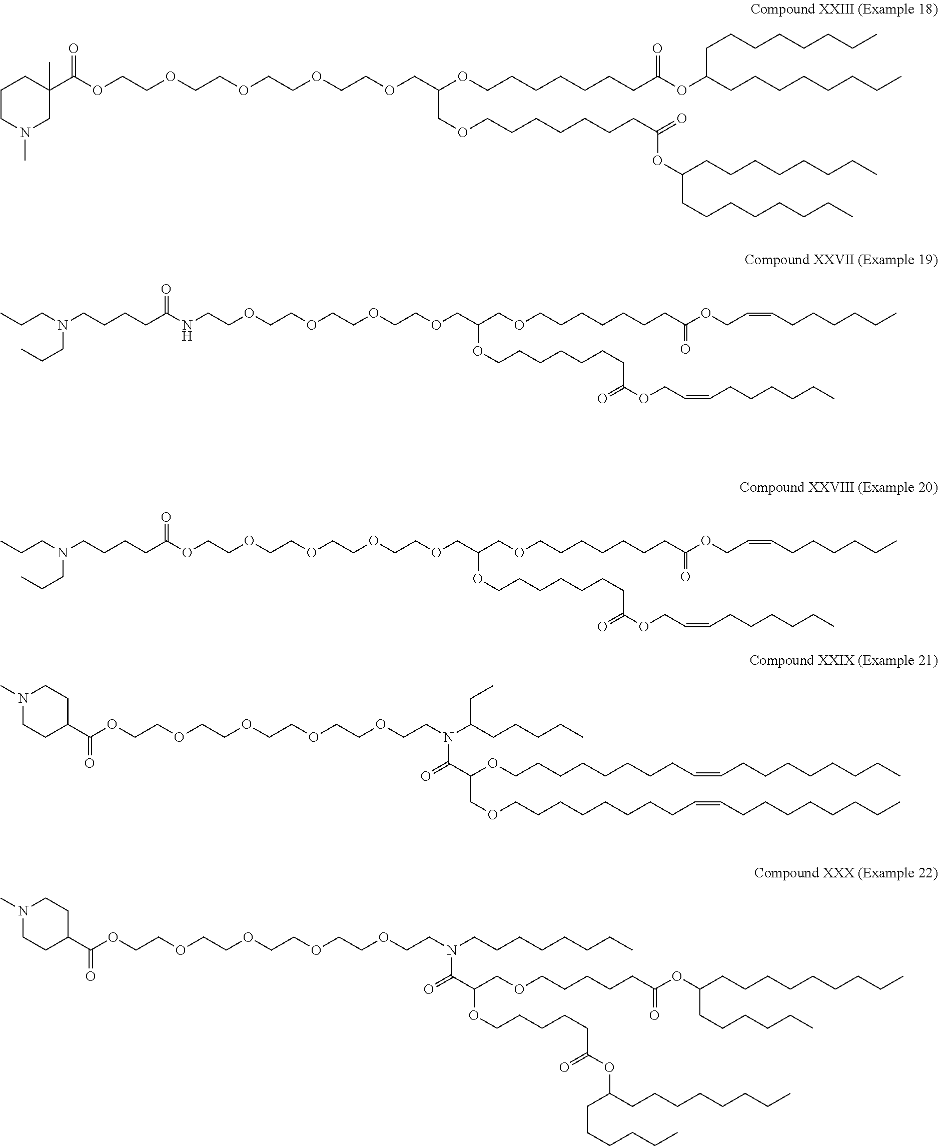 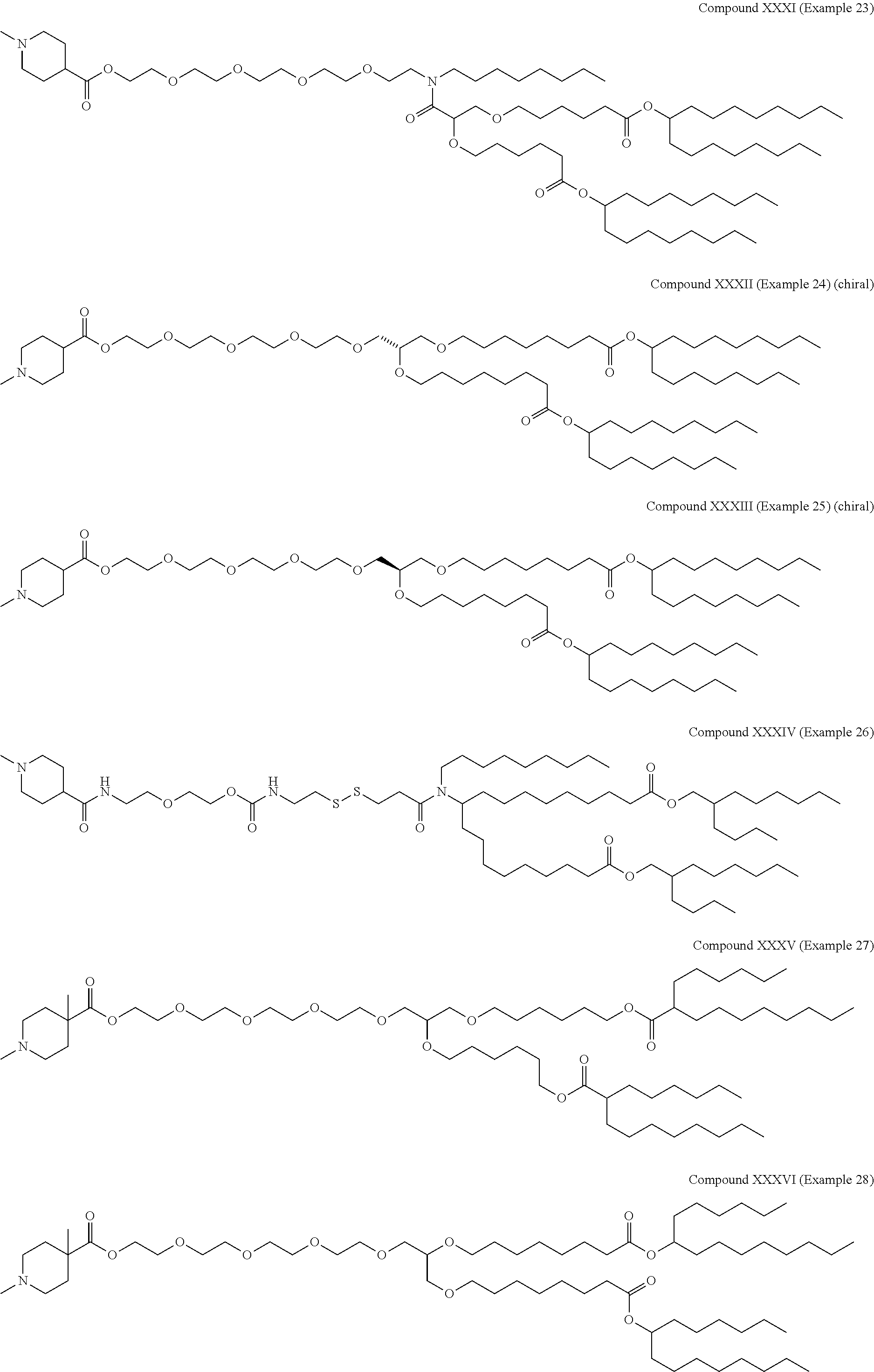 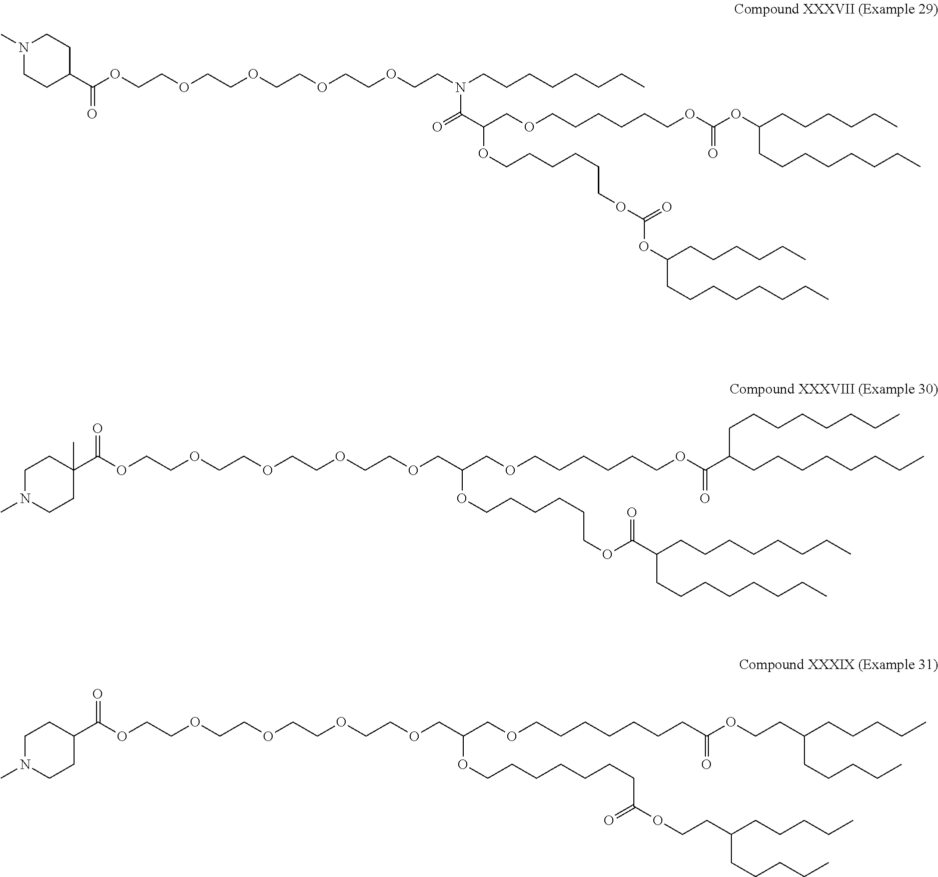 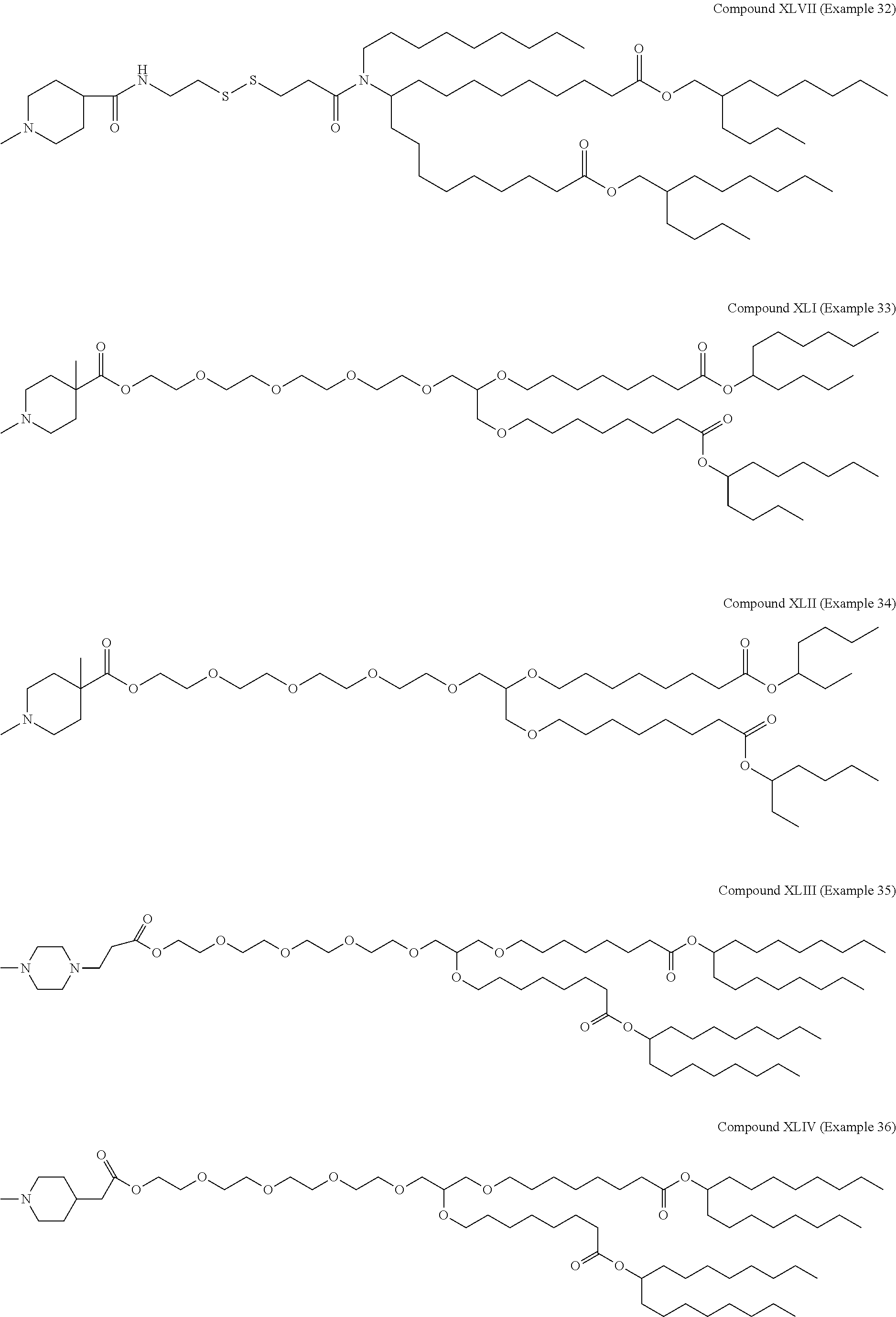 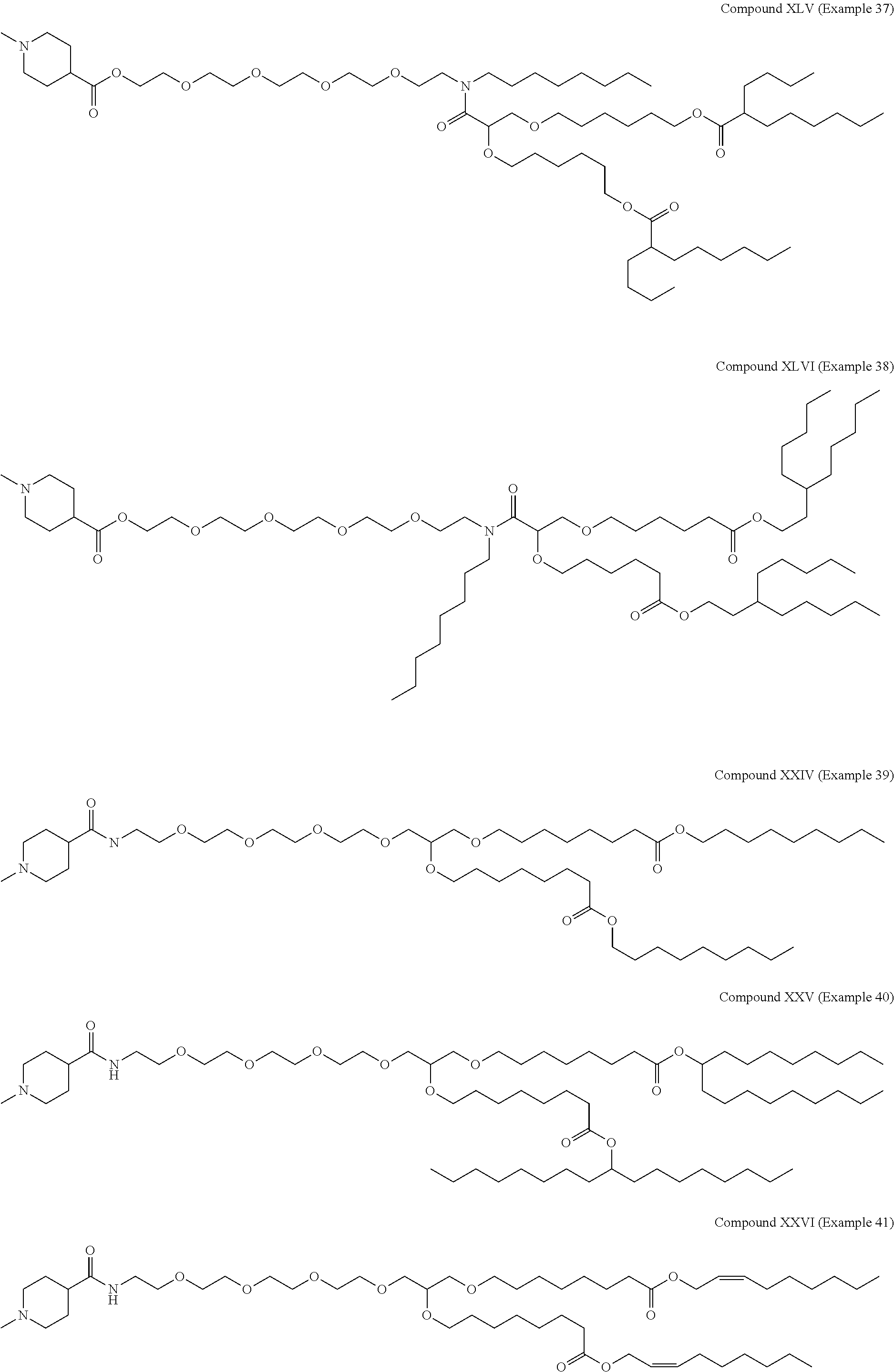
or one of its pharmaceutically acceptable salts thereof; and with said compound that is in all the possible racemic, enantiomeric and diastereoisomeric isomer forms and in particular in the group consisting of compounds (VI), (VII), (VIII), (XI), (XII), (XIV), (XV), (XVI), (XVII), (XVIII), (XIX), (XX), (XXI), (XXX), (XXXI), (XXXII), (XXXIII), (XXXIV), (XXXV), (XXXVI), (XXXVII) or (XXXVIIII).

18. A composition or a lipid nanoparticle (LNP) comprising a lipid component comprising at least a lipidic compound according to anyone of claims 1 to 17.

19. The composition or the LNP according to claim 18, wherein the lipid component further comprises at least a lipid selected from a neutral lipid, a structural lipid, and optionally a PEG-lipid.

20. The composition or the LNP according to claim 19, wherein the neutral lipid is selected from the group consisting of phosphatidylcholines, such as DSPC, DPPC, DMPC, POPC, DOPC; phosphatidylethanolamines, such as DOPE, DPPE, DMPE, DSPE, DLPE; DEPE; DPPS; DOPG; sphingomyelins; and ceramides; and mixtures thereof.

21. The composition or the LNP according to claim 19 or 20, wherein the structural lipid is selected from the group consisting of a sterol or an ester thereof, tomatine, alpha-tocopherol, and corticosteroid, and mixtures thereof.

22. The composition or the LNP according of claim 21, wherein the sterol or ester thereof is selected from the group consisting of cholesterol and its derivatives, ergosterol, desmosterol, stigmasterol, lanosterol, 7-dehydrocholesterol, dihydrolanosterol, zymosterol, lathosterol, diosgenin, sitosterol, sitostanol, campesterol, fecosterol, brassicasterol, tomatidine, ursolic acid, 24-methylene cholesterol, cholesteryl margarate, cholesteryl oleate, and cholesteryl stearate, and mixtures thereof.

23. The composition or the LNP according to anyone of claims 19 to 22, wherein the PEG-lipid is selected from the group consisting of PEG-DAG, DMG-PEG, PEG-PE, PEG-S-DAG, PEG-S-DMG, DSPC-PEG, DSPE-PEG, PEG-cer, mPEG-N,N-ditetradecylacetamide, a PEG-dialkyoxypropylcarbamate, and mixtures thereof.

24. The composition or the LNP according to anyone of claims 19 to 23, comprising at least a lipidic compound in a molar amount of about 30% to about 70%, a neutral lipid in a molar amount of about 0% to about 50%, a structural lipid in a molar amount of about 20% to about 50%, and a PEG-lipid in a molar amount of about 1% to about 15%, in % relative to the total molar amount of the lipid component.

25. The composition or the LNP according to anyone of claims 19 to 24, further comprising at least one biologically active agent.

26. The composition or the LNP according to claim 25, wherein the biologically active agent is a nucleic acid.

27. The composition or the LNP according to claim 26, wherein the nucleic acid encodes at least an antigen.

28. A pharmaceutical composition comprising at least a composition or a LNP according to claim 26 or 27, and a pharmaceutically acceptable excipient.

29. An immunogenic composition comprising at least a composition or a LNP according to claim 28.

30. A composition or a LNP according to anyone of claims 26 to 28, for use as a medicament.

31. A composition or a LNP according to claim 26 or 29, for use in a method for preventing and/or treating a disease selected in a group consisting of infectious diseases, allergies, autoimmune diseases, blood disorders, metabolic diseases, neurologic diseases, and cancer diseases.
